SEQUENCE LISTING (1038 sequences)

SEQ ID NO: 1 - protein, 5 aa, synthetic construct
SIFNT

SEQ ID NO: 2 - protein, 7 aa, synthetic construct
LYKAGEL

SEQ ID NO: 3 - protein, 13 aa, synthetic construct
CAGRNFGNEKLTF

SEQ ID NO: 4 - protein, 6 aa, synthetic construct
DFQATT

SEQ ID NO: 5 - protein, 7 aa, synthetic construct
SNEGSKA

SEQ ID NO: 6 - protein, 17 aa, synthetic construct
SARDRGLVSLPSVEAFF

SEQ ID NO: 7 - DNA, 387 bp, synthetic construct
  source 1..387: mol_type = other DNA
atgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctgggagaaactttggaaatgagaaattaacctttgggactggaacaagactcaccatcataccc

SEQ ID NO: 8 - DNA, 387 bp, synthetic construct; identical to SEQ ID NO: 7
  source 1..387: mol_type = other DNA

SEQ ID NO: 9 - protein, 129 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGRNFGNEKLTFGTGTRLTIIP

SEQ ID NO: 10 - DNA, 405 bp, synthetic construct
  source 1..405: mol_type = other DNA
atgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctcgcgacagggggcttgtatcgttgccgtcggtagaagctttctttggacaaggcaccagactcacagttgtactg

SEQ ID NO: 11 - DNA, 405 bp, synthetic construct; identical to SEQ ID NO: 10
  source 1..405: mol_type = other DNA

SEQ ID NO: 12 - protein, 134 aa, synthetic construct
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARDRGLVSLPSVEAFFGQGTRLTVV

SEQ ID NO: 13 - protein, 270 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGRNFGNEKLTFGTGTRLTIIPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 312 aa, synthetic construct
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARDRGLVSLPSVEAFFGQGTRLTVVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 15 - protein, 10 aa, synthetic construct
KLVVVGACGV

SEQ ID NO: 16 - protein, 6 aa, synthetic construct
VSGNPY

SEQ ID NO: 17 - protein, 8 aa, synthetic construct
YITGDNLV

SEQ ID NO: 18 - protein, 14 aa, synthetic construct
CAVRDQSGANNLFF

SEQ ID NO: 19 - protein, 5 aa, synthetic construct
SEHNR

SEQ ID NO: 20 - protein, 6 aa, synthetic construct
FQNEAQ

SEQ ID NO: 21 - protein, 14 aa, synthetic construct
ASYLSGSIYNEQFF

SEQ ID NO: 22 - DNA, 399 bp, synthetic construct
  source 1..399: mol_type = other DNA
atggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagaccaaagtggggcaaacaacctcttctttgggactggaacgagactcaccgttattccc

SEQ ID NO: 23 - DNA, 399 bp, synthetic construct
  source 1..399: mol_type = other DNA
atggcttctgcgcctatatcaatgcttgccatgctgtttacactgtccggtctgagggctcaaagcgtggcccaacctgaggatcaggtgaatgtagcggagggcaatccgttgacagttaagtgtacatactccgtatcaggcaatccgtacttgttttggtatgtgcagtaccccaatcgggggcttcaattcttgctgaagtacattacaggcgataatctggtaaaaggtagttatggttttgaggccgaattcaacaaatcacaaacatcatttcatcttaaaaagccaagcgcacttgtcagtgactcagcgctttatttctgtgcagtcagagaccaatcaggggcaaataatctgttctttgggacagggactagattgactgttataccc

SEQ ID NO: 24 - protein, 133 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDQSGANNLFFGTGTRLTVIP

SEQ ID NO: 25 - DNA, 402 bp, synthetic construct
  source 1..402: mol_type = other DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagctacctgagcggttccatttacaatgagcagttcttcgggccagggacacggctcaccgtgcta

SEQ ID NO: 26 - DNA, 402 bp, synthetic construct
  source 1..402: mol_type = other DNA
atgggcactagcctcttgtgttggatggcactttgccttcttggcgcggatcacgccgatacaggcgtctcccaagatcccagacataaaatcacaaaacggggccagaacgttacctttcgctgcgatccgatatcagagcataatcgactgtattggtataggcaaactctcgggcaagggcctgagttcctcacttatttccaaaatgaggcgcaactggaaaagagccggttgttgagtgataggttttccgcagagcgacccaaggggagcttctcaacactggagatacaaaggaccgaacaaggtgattccgcaatgtatctctgtgctagttatttgagcggctccatatataacgaacagtttttcggaccgggcactcgcctgaccgtacta

SEQ ID NO: 27 - protein, 134 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASYLSGSIYNEQFFGPGTRLTVL

SEQ ID NO: 28 - protein, 274 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDQSGANNLFFGTGTRLTVIPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 29 - protein, 311 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASYLSGSIYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 30 - protein, 9 aa, synthetic construct
LVVVGACGV

SEQ ID NO: 31 - protein, 6 aa, synthetic construct
NSMFDY

SEQ ID NO: 32 - protein, 7 aa, synthetic construct
ISSIKDK

SEQ ID NO: 33 - protein, 12 aa, synthetic construct
AASGGGGADGLT

SEQ ID NO: 34 - protein, 5 aa, synthetic construct
MNHNY

SEQ ID NO: 35 - protein, 6 aa, synthetic construct
SVGAGI

SEQ ID NO: 36 - protein, 12 aa, synthetic construct
ASSYSTERGTIY

SEQ ID NO: 37 - DNA, 417 bp, synthetic construct
  source 1..417: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgggggaggaggtgctgacggactcacctttggcaaagggactcatctaatcatccagccc

SEQ ID NO: 38 - DNA, 417 bp, synthetic construct
  source 1..417: mol_type = other DNA
atggccatgctgctgggcgccagcgtgctgattttatggctgcagcccgactgggtgaacagccagcagaagaacgacgaccagcaagtgaagcagaactccccttctttaagcgtgcaagaaggtcgtatcagcattttaaactgcgactacaccaacagcatgttcgactactttttatggtacaagaagtaccccgccgagggccccacctttttaatcagcatcagcagcatcaaggacaagaacgaggacggtcgtttcaccgtgtttttaaacaagagcgccaagcatttatctttacacatcgtgccctcccagcccggtgatagcgccgtgtacttctgcgccgccagcggaggaggaggcgccgatggactgaccttcggcaagggcacccatttaatcatccagccc

SEQ ID NO: 39 - protein, 139 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGGGGADGLTFGKGTHLIIQP

SEQ ID NO: 40 - DNA, 486 bp, synthetic construct
  source 1..486: mol_type = other DNA
atgaggtctcagaatgacttccttgagagtcctgttcccctttcatcaatgcacagatacagaagacccctccgtcctggagcacctgccatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagttactcgacggaacgcgggaccatatattttggagagggaagttggctcactgttgta

SEQ ID NO: 41 - DNA, 486 bp, synthetic construct
  source 1..486: mol_type = other DNA
atgaggagccagaacgactttttagagagccccgtgcctctgagcagcatgcataggtataggaggcctctgagacccggtgcccccgctatgagcatctctttactgtgctgtgctgcctttcctttactgtgggctggccccgttaacgctggcgtgacccagacccccaagtttaggattttaaagatcggccagtccatgactttacagtgcacccaagatatgaaccacaactacatgtactggtatcgtcaagatcccggcatgggtttaaagctgatttactacagcgtgggagccggcatcaccgacaagggcgaggtgcccaacggctacaatgtgtctcgtagcaccaccgaggacttccctctgagactggagctggccgcccctagccagacaagcgtgtacttctgcgcctcctcctacagcaccgagaggggcaccatctacttcggcgagggcagctggctgaccgtggtg

SEQ ID NO: 42 - protein, 162 aa, synthetic construct
MRSQNDFLESPVPLSSMHRYRRPLRPGAPAMSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSTERGTIYFGEGSWLTVV

SEQ ID NO: 43 - protein, 280 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGGGGADGLTFGKGTHLIIQPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 339 aa, synthetic construct
MRSQNDFLESPVPLSSMHRYRRPLRPGAPAMSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSTERGTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 45 - protein, 9 aa, synthetic construct
VVGAVGVGK

SEQ ID NO: 46 - protein, 10 aa, synthetic construct
VVVGAVGVGK

SEQ ID NO: 47 - protein, 5 aa, synthetic construct
TSINN

SEQ ID NO: 48 - protein, 7 aa, synthetic construct
IRSNERE

SEQ ID NO: 49 - protein, 12 aa, synthetic construct
ATDRQSSGDKLT

SEQ ID NO: 50 - protein, 5 aa, synthetic construct
SGHAT

SEQ ID NO: 51 - protein, 6 aa, synthetic construct
FQNNGV

SEQ ID NO: 52 - protein, 11 aa, synthetic construct
ASSLADIYEQY

SEQ ID NO: 53 - DNA, 396 bp, synthetic construct
  source 1..396: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggaccgtcaaagcagcggagacaagctgacttttgggaccgggactcgtttagcagttaggccc

SEQ ID NO: 54 - DNA, 396 bp, synthetic construct
  source 1..396: mol_type = other DNA
atggagactttactgggcgtgtctttagtgattttatggctgcagctggctcgtgtgaatagccagcaaggtgaagaggacccccaagctttaagcatccaagaaggcgagaacgccaccatgaactgctcctacaagaccagcatcaacaatttacagtggtatcgtcagaacagcggtcgtggtttagtgcatttaattttaattcgtagcaacgagagggagaagcacagcggtcgtctgagggtgactttagacaccagcaagaagagcagctctttactgatcacagcctctagggccgctgacaccgctagctacttctgcgccaccgacagacagagcagcggcgacaagctgaccttcggcaccggcacaagactggccgtgagaccc

SEQ ID NO: 55 - protein, 132 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDRQSSGDKLTFGTGTRLAVRP

SEQ ID NO: 56 - DNA, 396 bp, synthetic construct
  source 1..396: mol_type = other DNA
atgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagcttagccgacatctacgagcagtacttcgggccgggcaccaggctcacggtcaca

SEQ ID NO: 57 - DNA, 396 bp, synthetic construct
  source 1..396: mol_type = other DNA
atgggcaccagactgctgtgctgggccgctctgtgtctgctgggcgctgagctgacagaagctggcgtggcccagagccctcgttacaagatcatcgagaagaggcagagcgtggccttctggtgcaaccccatcagcggccacgccactttatactggtaccagcagattttaggccaaggtcccaagctgctgatccagttccagaacaacggcgtggtggacgacagccagctgcccaaggatcgtttcagcgccgagaggctgaagggcgtggacagcactttaaaaatccagcccgctaagctggaggacagcgccgtgtatttatgcgctagctctttagccgacatctacgagcagtacttcggccccggcactcgtctgaccgtgacc

SEQ ID NO: 58 - protein, 132 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLADIYEQYFGPGTRLTVT

SEQ ID NO: 59 - protein, 273 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDRQSSGDKLTFGTGTRLAVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 309 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLADIYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 61 - protein, 9 aa, synthetic construct
VVGAVGVGK

SEQ ID NO: 62 - protein, 10 aa, synthetic construct
VVVGAVGVGK

SEQ ID NO: 63 - protein, 6 aa, synthetic construct
TSGFNG

SEQ ID NO: 64 - protein, 6 aa, synthetic construct
NVLDGL

SEQ ID NO: 65 - protein, 12 aa, synthetic construct
CAPGDNFNKFYF

SEQ ID NO: 66 - protein, 5 aa, synthetic construct
SGHRS

SEQ ID NO: 67 - protein, 6 aa, synthetic construct
YFSETQ

SEQ ID NO: 68 - protein, 12 aa, synthetic construct
CASSARNDEAFF

SEQ ID NO: 69 - DNA, 378 bp, synthetic construct
  source 1..378: mol_type = other DNA
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctcccggggacaacttcaacaaattttactttggatctgggaccaaactcaatgtaaaacca

SEQ ID NO: 70 - DNA, 378 bp, synthetic construct
  source 1..378: mol_type = other DNA
atgtggggcgtgtttctgctgtacgtgtccatgaagatgggcggcaccacaggccagaacatcgaccagccaaccgagatgaccgccacagagggcgccatcgtgcagatcaactgcacctaccagacatctggcttcaatggcctgttttggtatcagcagcacgcaggagaggcacccacattcctgagctataatgtgctggatggcctggaggagaagggcaggttctcctcttttctgtctcgcagcaagggctactcctatctgctgctgaaggagctgcagatgaaggactccgcctcttacctgtgcgcccctggcgataactttaataagttctatttcggctctggcaccaagctgaatgtgaagcca

SEQ ID NO: 71 - protein, 126 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAPGDNFNKFYFGSGTKLNVKP

SEQ ID NO: 72 - DNA, 390 bp, synthetic construct
  source 1..390: mol_type = other DNA
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcgcgagaaatgatgaagctttctttggacaaggcaccagactcacagttgta

SEQ ID NO: 73 - DNA, 390 bp, synthetic construct
  source 1..390: mol_type = other DNA
atgggcagccggctgctgtgctgggtgctgctgtgcctgctgggagcaggaccagtgaaggcaggcgtgacccagacacctcggtacctgatcaagaccagaggccagcaggtgacactgagctgctccccaatctccggccacagatctgtgagctggtaccagcagaccccaggacagggactgcagttcctgtttgagtatttctccgagacacagaggaacaagggcaatttccctggccggttttctggcagacagttttccaactctcgcagcgagatgaatgtgagcaccctggagctgggcgactccgccctgtacctgtgcgccagctccgccaggaacgatgaggccttctttggccagggcacccggctgacagtggtg

SEQ ID NO: 74 - protein, 130 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSARNDEAFFGQGTRLTVV

SEQ ID NO: 75 - protein, 267 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAPGDNFNKFYFGSGTKLNVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 76 - protein, 307 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSARNDEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 77 - protein, 9 aa, synthetic construct
VVGAVGVGK

SEQ ID NO: 78 - protein, 10 aa, synthetic construct
VVVGAVGVGK

SEQ ID NO: 79 - protein, 6 aa, synthetic construct
DRGSQS

SEQ ID NO: 80 - protein, 6 aa, synthetic construct
IYSNGD

SEQ ID NO: 81 - protein, 14 aa, synthetic construct
CAVKSRAGSYQLTF

SEQ ID NO: 82 - protein, 5 aa, synthetic construct
SGHNS

SEQ ID NO: 83 - protein, 6 aa, synthetic construct
FNNNVP

SEQ ID NO: 84 - protein, 12 aa, synthetic construct
CASSLGDSEQYF

SEQ ID NO: 85 - DNA, 399 bp, synthetic construct
  source 1..399: mol_type = other DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaagtcaagggctgggagttaccaactcactttcgggaaggggaccaaactctcggtcatacca

SEQ ID NO: 86 - DNA, 399 bp, synthetic construct
  source 1..399: mol_type = other DNA
atgaagagcctgcgggtgctgctggtcatcctgtggctgcagctgtcctgggtgtggtctcagcagaaggaggtggagcagaatagcggaccactgtccgtgccagagggagccatcgcctccctgaactgcacatactctgacaggggctcccagtctttcttttggtaccgccagtatagcggcaagtcccccgagctgatcatgttcatctactctaatggcgacaaggaggatggcaggtttaccgcccagctgaacaaggcctctcagtatgtgagcctgctgatccgcgacagccagcctagcgattccgccacatacctgtgcgcagtgaagtcccgggcaggctcttatcagctgacctttggcaagggcacaaagctgagcgtgatccca

SEQ ID NO: 87 - protein, 133 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKSRAGSYQLTFGKGTKLSVIP

SEQ ID NO: 88 - DNA, 393 bp, synthetic construct
  source 1..393: mol_type = other DNA
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtctcggggacagcgagcagtacttcgggccgggcaccaggctcacggtcaca

SEQ ID NO: 89 - DNA, 393 bp, synthetic construct
  source 1..393: mol_type = other DNA
atggacagctggaccttctgctgcgtgagcctgtgcatcctggtggccaagcacacagatgcaggcgtgatccagtccccaaggcacgaggtgaccgagatgggacaggaggtgacactgaggtgtaagcctatctctggccacaatagcctgttctggtacaggcagaccatgatgcgcggcctggagctgctgatctacttcaacaataacgtgcctatcgacgattccggcatgccagaggacagattctctgccaagatgcccaacgcctccttttctacactgaagatccagccaagcgagcctagggactccgccgtgtacttctgcgccagctccctgggcgatagcgagcagtattttggccctggcacccggctgaccgtgaca

SEQ ID NO: 90 - protein, 131 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGDSEQYFGPGTRLTVT

SEQ ID NO: 91 - protein, 274 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKSRAGSYQLTFGKGTKLSVIPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - protein, 308 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGDSEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 93 - protein, 9 aa, synthetic construct
VVGAVGVGK

SEQ ID NO: 94 - protein, 10 aa, synthetic construct
VVVGAVGVGK

SEQ ID NO: 95 - protein, 6 aa, synthetic construct
SSYSPS

SEQ ID NO: 96 - protein, 8 aa, synthetic construct
YTSAATLV

SEQ ID NO: 97 - protein, 16 aa, synthetic construct
CVVSGGGSSNTGKLIF

SEQ ID NO: 98 - protein, 5 aa, synthetic construct
SGHAT

SEQ ID NO: 99 - protein, 6 aa, synthetic construct
FQNNGV

SEQ ID NO: 100 - protein, 14 aa, synthetic construct
CASSQRSNTGELFF

SEQ ID NO: 101 - DNA, 405 bp, synthetic construct
  source 1..405: mol_type = other DNA
atgctcctgctgctcgtcccagtgctcgaggtgatttttactctgggaggaaccagagcccagtcggtgacccagcttgacagccacgtctctgtctctgaaggaaccccggtgctgctgaggtgcaactactcatcttcttattcaccatctctcttctggtatgtgcaacaccccaacaaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgttgtgagtgggggaggctctagcaacacaggcaaactaatctttgggcaagggacaactttacaagtaaaacca

SEQ ID NO: 102 - DNA, 405 bp, synthetic construct
  source 1..405: mol_type = other DNA
atgctgctgctgctggtgcccgtgctggaagtgatcttcaccctgggaggaacaagggcacagagcgtgacccagctggactcccacgtgtccgtgtctgagggcacacccgtgctgctgagatgcaactactcctctagctatagcccctccctgttctggtacgtgcagcaccctaataagggcctgcagctgctgctgaagtatacctccgccgccacactggtgaagggcatcaacggcttcgaggccgagtttaagaagagcgagacctccttccacctgacaaagccttctgcccacatgagcgatgccgccgagtacttttgcgtggtgagcggcggcggctcctctaataccggcaagctgatcttcggccagggcaccacactgcaggtgaagcca

SEQ ID NO: 103 - protein, 135 aa, synthetic construct
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVSGGGSSNTGKLIFGQGTTLQVKP

SEQ ID NO: 104 - DNA, 399 bp, synthetic construct
  source 1..399: mol_type = other DNA
atgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagccagaggtcgaacaccggggagctgttttttggagaaggctctaggctgaccgtactg

SEQ ID NO: 105 - DNA, 399 bp, synthetic construct
  source 1..399: mol_type = other DNA
atgggcacccggctgctgtgctgggccgccctgtgcctgctgggagcagagctgacagaggcaggagtggcccagtccccacggtacaagatcatcgagaagagacagtccgtggccttttggtgcaaccccatctctggccacgccaccctgtactggtatcagcagatcctgggccagggccctaagctgctgatccagttccagaacaatggcgtggtggacgattctcagctgccaaaggacaggtttagcgccgagcgcctgaagggcgtggatagcaccctgaagatccagcctgccaagctggaggacagcgccgtgtatctgtgcgccagctcccagcggtccaatacaggcgagctgttctttggcgagggctctaggctgaccgtgctg

SEQ ID NO: 106 - protein, 133 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSQRSNTGELFFGEGSRLTVL

SEQ ID NO: 107 - protein, 276 aa, synthetic construct
MLLLLVPVLEVIFTLGGTRAQSVTQLDSHVSVSEGTPVLLRCNYSSSYSPSLFWYVQHPNKGLQLLLKYTSAATLVKGINGFEAEFKKSETSFHLTKPSAHMSDAAEYFCVVSGGGSSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 108 - protein, 310 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSQRSNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 109 - protein, 9 aa, synthetic construct
VVGACGVGK

SEQ ID NO: 110 - protein, 10 aa, synthetic construct
VVVGACGVGK

SEQ ID NO: 111 - protein, 5 aa, synthetic construct
TSINN

SEQ ID NO: 112 - protein, 7 aa, synthetic construct
IRSNERE

SEQ ID NO: 113 - protein, 14 aa, synthetic construct
CATDAGGGADGLTF

SEQ ID NO: 114 - protein, 5 aa, synthetic construct
SGDLS

SEQ ID NO: 115 - protein, 6 aa, synthetic construct
YYNGEE

SEQ ID NO: 116 - protein, 14 aa, synthetic construct
CASGGRDSTDTQYF

SEQ ID NO: 117 - DNA, 396 bp, synthetic construct
  source 1..396: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgccggaggaggtgctgacggactcacctttggcaaagggactcatctaatcatccagccc

SEQ ID NO: 118 - DNA, 396 bp, synthetic construct
  source 1..396: mol_type = other DNA
atggagacactgctgggcgtgtccctggtcatcctgtggctgcagctggccagggtgaacagccagcagggagaggaggacccccaggccctgtctatccaggagggcgagaacgccaccatgaattgctcttacaagacaagcatcaacaatctgcagtggtatagacagaactccggcaggggcctggtgcacctgatcctgatccgctccaatgagcgggagaagcactctggccggctgagagtgaccctggatacatctaagaagtcctctagcctgctgatcaccgccagccgggcagcagacacagcctcctacttttgtgccaccgatgccgggggcggagcagacggactgacattcgggaaggggactcacctgattatccagcca

SEQ ID NO: 119 - protein, 132 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAGGGADGLTFGKGTHLIIQP

SEQ ID NO: 120 - DNA, 396 bp, synthetic construct
  source 1..396: mol_type = other DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcgggggacgggattccacagatacgcagtattttggcccaggcacccggctgacagtgctc

SEQ ID NO: 121 - DNA, 396 bp, synthetic construct
  source 1..396: mol_type = other DNA
atgggctttcggctgctgtgctgcgtggctttttgcctgctgggggctgggcctgtggatagcggggtcactcagacacctaaacatctgatcaccgcaacaggacagagggtgaccctgaggtgctctcctcggagcggcgacctgagcgtgtactggtatcagcagagcctggatcagggcctgcagttcctgatccagtactataacggcgaggagcgcgccaagggcaatatcctggagcggttctctgcccagcagtttccagacctgcacagcgagctgaacctgagctccctggagctgggcgatagcgccctgtacttctgcgcctccggcggcagagactctaccgatacacagtattttggccccggcaccagactgacagtgctg

SEQ ID NO: 122 - protein, 132 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASGGRDSTDTQYFGPGTRLTVL

SEQ ID NO: 123 - protein, 273 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAGGGADGLTFGKGTHLIIQPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 124 - protein, 309 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASGGRDSTDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 125 - protein, 9 aa, synthetic construct
VVGAVGVGK

SEQ ID NO: 126 - protein, 10 aa, synthetic construct
VVVGAVGVGK

SEQ ID NO: 127-218 - skipped (92 entries)

SEQ ID NO: 219 - protein, 10 aa, synthetic construct
KLVVVGADGV

SEQ ID NO: 220 - protein, 9 aa, synthetic construct
LVVVGADGV

SEQ ID NO: 221 - protein, 10 aa, synthetic construct
KLVVVGAVGV

SEQ ID NO: 222 - protein, 9 aa, synthetic construct
LVVVGAVGV

SEQ ID NO: 223-238 - skipped (16 entries)

SEQ ID NO: 239 - protein, 5 aa, synthetic construct
SVFSS

SEQ ID NO: 240 - protein, 7 aa, synthetic construct
VVTGGEV

SEQ ID NO: 241 - protein, 11 aa, synthetic construct
AGGPNTGNQFY

SEQ ID NO: 242 - protein, 5 aa, synthetic construct
SGHNT

SEQ ID NO: 243 - protein, 6 aa, synthetic construct
YYREEE

SEQ ID NO: 244 - protein, 14 aa, synthetic construct
ASSTSFWEVNTEAF

SEQ ID NO: 245 - DNA, 390 bp, synthetic construct
  source 1..390: mol_type = other DNA
atggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagggccgaacaccggtaaccagttctattttgggacagggacaagtttgacggtcattccaaat

SEQ ID NO: 246 - DNA, 390 bp, synthetic construct
  source 1..390: mol_type = other DNA
atggtgctgaagttttccgtgtctatcctgtggattcagctggcctgggtgtctacccagctgctggagcagagcccccagttcctgtccatccaggagggcgagaacctgacagtgtactgcaattctagctccgtgttttctagcctgcagtggtataggcaggagccaggagagggacccgtgctgctggtgaccgtggtgacaggcggcgaggtgaagaagctgaagagactgaccttccagtttggcgacgccaggaaggattcctctctgcacatcaccgcagcacagcctggcgatacaggactgtacctgtgcgcaggaggaccaaacaccggcaatcagttctattttggcaccggcacatccctgacagtgatccctaat

SEQ ID NO: 247 - protein, 130 aa, synthetic construct
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGGPNTGNQFYFGTGTSLTVIPN

SEQ ID NO: 248 - DNA, 402 bp, synthetic construct
  source 1..402: mol_type = other DNA
atgggccctgggctcctctgctgggtgctgctttgtctcctgggagcaggctcagtggagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagcacatctttttgggaggtgaacactgaagctttctttggacaaggcaccagactcacagttgta

SEQ ID NO: 249 - DNA, 402 bp, synthetic construct
  source 1..402: mol_type = other DNA
atgggaccaggactgctgtgctgggtgctgctgtgcctgctgggagcaggctccgtggagaccggcgtgacacagtctcccacccacctgatcaagacaagaggccagcaggtgaccctgaggtgcagctcccagtctggccacaacacagtgagctggtaccagcaggccctgggacagggacctcagttcatctttcagtactatagggaggaggagaacggccgcggcaatttcccccctcggtttagcggcctgcagttcccaaactactctagcgagctgaacgtgaatgccctggagctggacgatagcgccctgtatctgtgcgcctcctctacctccttttgggaagtgaatacagaggccttctttggccagggcacccgcctgacagtggtg

SEQ ID NO: 250 - protein, 134 aa, synthetic construct
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSTSFWEVNTEAFFGQGTRLTVV

SEQ ID NO: 251 - protein, 267 aa, synthetic construct
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGGPNTGNQFYFGTGTSLTVIPNDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 252 - protein, 307 aa, synthetic construct
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSTSFWEVNTEAFFGQGTRLTVVKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 253 - protein, 9 aa, synthetic construct
VVGACGVGK

SEQ ID NO: 254 - protein, 10 aa, synthetic construct
VVVGACGVGK

SEQ ID NO: 255 - protein, 6 aa, synthetic construct
TSGFNG

SEQ ID NO: 256 - protein, 6 aa, synthetic construct
NVLDGL

SEQ ID NO: 257 - protein, 15 aa, synthetic construct
AVREEVLYNQGGKLI

SEQ ID NO: 258 - protein, 5 aa, synthetic construct
LNHDA

SEQ ID NO: 259 - protein, 6 aa, synthetic construct
SQIVND

SEQ ID NO: 260 - protein, 12 aa, synthetic construct
ASSKRGWPYEQY

SEQ ID NO: 261 - DNA, 393 bp, synthetic construct
  source 1..393: mol_type = other DNA
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagaggaggtcctttataaccagggaggaaagcttatcttcggacagggaacggagttatctgtgaaaccc

SEQ ID NO: 262 - DNA, 393 bp, synthetic construct
  source 1..393: mol_type = other DNA
atgtggggcgtgtttctgctgtacgtgagcatgaagatgggcggcaccacaggccagaacatcgaccagccaaccgagatgaccgccacagagggcgccatcgtgcagatcaactgcacctaccagacaagcggcttcaatggcctgttttggtatcagcagcacgcaggagaggcacccacattcctgtcctataatgtgctggacggcctggaggagaagggcaggttctcctcttttctgagccgctccaagggctactcctatctgctgctgaaggagctgcagatgaaggattctgccagctacctgtgcgccgtgcgggaggaggtgctgtataatcagggcggcaagctgatctttggccagggcaccgagctgagcgtgaagcct

SEQ ID NO: 263 - protein, 131 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVREEVLYNQGGKLIFGQGTELSVKP

SEQ ID NO: 264 - DNA, 396 bp, synthetic construct
  source 1..396: mol_type = other DNA
atgagcaaccaggtgctctgctgtgtggtcctttgtctcctgggagcaaacaccgtggatggtggaatcactcagtccccgaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtaaaaggggatggccctacgagcagtacttcgggccgggcaccaggctcacggtcaca

SEQ ID NO: 265 - DNA, 396 bp, synthetic construct
  source 1..396: mol_type = other DNA
atgtccaaccaggtgctgtgctgcgtggtgctgtgcctgctgggagcaaataccgtggacggaggcatcacacagtcccccaagtacctgttccggaaggagggccagaacgtgaccctgtcttgtgagcagaacctgaatcacgacgccatgtactggtataggcaggaccccggacagggactgagactgatctactatagccagatcgtgaacgactttcagaagggcgacatcgccgagggctacagcgtgtcccgggagaagaaggagtccttcccactgaccgtgacatctgcccagaagaatcccaccgccttttatctgtgcgccagctccaagagaggctggccctacgagcagtatttcggccctggcaccaggctgaccgtgaca

SEQ ID NO: 266 - protein, 132 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSKRGWPYEQYFGPGTRLTVT

SEQ ID NO: 267 - protein, 268 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVREEVLYNQGGKLIFGQGTELSVKPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 268 - protein, 305 aa, synthetic construct
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSKRGWPYEQYFGPGTRLTVTKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 269 - protein, 9 aa, synthetic construct
VVGACGVGK

SEQ ID NO: 270 - protein, 10 aa, synthetic construct
VVVGACGVGK

SEQ ID NO: 271 - protein, 5 aa, synthetic construct
TSINN

SEQ ID NO: 272 - protein, 7 aa, synthetic construct
IRSNERE

SEQ ID NO: 273 - protein, 12 aa, synthetic construct
ATDRQSSGDKLT

SEQ ID NO: 274 - protein, 5 aa, synthetic construct
SGHAT

SEQ ID NO: 275 - protein, 6 aa, synthetic construct
FQNNGV

SEQ ID NO: 276 - protein, 11 aa, synthetic construct
ASSLADIYEQY

SEQ ID NO: 277 - DNA, 396 bp, synthetic construct; identical to SEQ ID NO: 53
  source 1..396: mol_type = other DNA

SEQ ID NO: 278 - DNA, 396 bp, synthetic construct
  source 1..396: mol_type = other DNA
atggagaccctgctgggcgtgtccctggtcatcctgtggctgcagctggccagggtgaacagccagcagggagaggaggacccccaggccctgagcatccaggagggcgagaacgccaccatgaattgctcttacaagacaagcatcaacaatctgcagtggtataggcagaactccggccgcggactggtgcacctgatcctgatccggagcaatgagagagagaagcactccggccggctgagagtgaccctggacacatctaagaagtcctctagcctgctgatcaccgcctctcgggcagcagatacagccagctacttctgtgccaccgacagacagtcctctggcgataagctgacctttggcaccggcacaaggctggccgtgcgcccc

SEQ ID NO: 279 - protein, 132 aa, synthetic construct; identical to SEQ ID NO: 55

SEQ ID NO: 280 - DNA, 396 bp, synthetic construct; identical to SEQ ID NO: 56
  source 1..396: mol_type = other DNA

SEQ ID NO: 281 - DNA, 396 bp, synthetic construct
  source 1..396: mol_type = other DNA
atgggaacaaggctgctgtgctgggccgccctgtgcctgctgggagcagagctgaccgaggccggcgtggcccagagcccccggtacaagatcatcgagaagagacagagcgtggccttctggtgcaaccctatctccggccacgccacactgtactggtatcagcagatcctgggccagggcccaaagctgctgatccagttccagaacaatggcgtggtggacgattcccagctgcccaaggaccggttttctgccgagagactgaagggcgtggattccaccctgaagatccagcccgccaagctggaggactctgccgtgtatctgtgcgccagctccctggccgacatctacgagcagtatttcggccctggcacaaggctgaccgtgaca

SEQ ID NO: 282 - protein, 132 aa, synthetic construct; identical to SEQ ID NO: 58

SEQ ID NO: 283 - protein, 269 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDRQSSGDKLTFGTGTRLAVRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 284 - protein, 305 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLADIYEQYFGPGTRLTVTKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 285 - protein, 9 aa, synthetic construct
VVGACGVGK

SEQ ID NO: 286 - protein, 10 aa, synthetic construct
VVVGACGVGK

SEQ ID NO: 287 - protein, 7 aa, synthetic construct
TSESDYY

SEQ ID NO: 288 - protein, 8 aa, synthetic construct
QEAYKQQN

SEQ ID NO: 289 - protein, 13 aa, synthetic construct
ALYIYGGSQGNLI

SEQ ID NO: 290 - protein, 5 aa, synthetic construct
SEHNR

SEQ ID NO: 291 - protein, 6 aa, synthetic construct
FQNEAQ

SEQ ID NO: 292 - protein, 11 aa, synthetic construct
ASSSTDRIEAF

SEQ ID NO: 293 - DNA, 408 bp, synthetic construct
  source 1..408: mol_type = other DNA
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgctctctatatttatggaggaagccaaggaaatctcatctttggaaaaggcactaaactctctgttaaacca

SEQ ID NO: 294 - DNA, 408 bp, synthetic construct
  source 1..408: mol_type = other DNA
atggcatgcccaggcttcctgtgggcactggtcatcagcacatgtctggagttttctatggcccagaccgtgacacagtctcagcctgagatgagcgtgcaggaggccgagaccgtgacactgagctgcacctacgacacatctgagagcgattactatctgttctggtataagcagccaccctccagacagatgatcctggtcatcaggcaggaggcctacaagcagcagaacgccaccgagaatcggttctccgtgaactttcagaaggccgccaagtccttttctctgaagatcagcgactcccagctgggcgatgccgccatgtatttctgtgccctgtacatctatggcggctctcagggcaatctgatctttggcaagggcaccaagctgagcgtgaagcct

SEQ ID NO: 295 - protein, 136 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCALYIYGGSQGNLIFGKGTKLSVKP

SEQ ID NO: 296 - DNA, 396 bp, synthetic construct
  source 1..396: mol_type = other DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagctccaccgacaggattgaagctttctttggacaaggcaccagactcacagttgta

SEQ ID NO: 297 - DNA, 396 bp, synthetic construct
  source 1..396: mol_type = other DNA
atgggcacctccctgctgtgctggatggcactgtgcctgctgggagcagaccacgcagatacaggcgtgtctcaggacccacgccacaagatcaccaagcggggccagaacgtgacattcagatgcgatcccatctccgagcacaataggctgtactggtataggcagaccctgggacagggaccagagttcctgacatactttcagaacgaggcccagctggagaagagccggctgctgtccgacagattctctgccgagaggcccaagggctcttttagcaccctggagatccagagaacagagcagggcgacagcgccatgtatctgtgcgccagctcctctaccgataggatcgaggccttctttggccagggcacccgcctgacagtggtg

SEQ ID NO: 298 - protein, 132 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSTDRIEAFFGQGTRLTVV

SEQ ID NO: 299 - protein, 273 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCALYIYGGSQGNLIFGKGTKLSVKPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 300 - protein, 305 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSTDRIEAFFGQGTRLTVVKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 301 - protein, 9 aa, synthetic construct
VVGADGVGK

SEQ ID NO: 302 - protein, 10 aa, synthetic construct
VVVGADGVGK

SEQ ID NO: 303 - protein, 6 aa, synthetic construct
VSNAYN

SEQ ID NO: 304 - protein, 4 aa, synthetic construct
GSKP

SEQ ID NO: 305 - protein, 9 aa, synthetic construct
ATYNFNKFY

SEQ ID NO: 306 - protein, 5 aa, synthetic construct
SGHTS

SEQ ID NO: 307 - protein, 6 aa, synthetic construct
YDEGEE

SEQ ID NO: 308 - protein, 15 aa, synthetic construct
ASTTFKTGRAIEKLF

SEQ ID NO: 309 - DNA, 387 bp, synthetic construct
  source 1..387: mol_type = other DNA
atggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctacgtacaacttcaacaaattttactttggatctgggaccaaactcaatgtaaaacca

SEQ ID NO: 310 - DNA, 387 bp, synthetic construct
  source 1..387: mol_type = other DNA
atggccctgcagtctacactgggagccgtgtggctgggactgctgctgaactctctgtggaaggtggccgagagcaaggaccaggtgttccagcctagcaccgtggcctcctctgagggagcagtggtggagatcttttgcaatcactccgtgtctaacgcctacaatttcttttggtatctgcactttccaggatgtgcaccaaggctgctggtgaagggcagcaagccatcccagcagggccggtacaacatgacctatgagagattcagctcctctctgctgatcctgcaggtgagagaggccgatgccgccgtgtactattgtgccacctacaactttaataagttctattttggctccggcacaaagctgaatgtgaagcct

SEQ ID NO: 311 - protein, 129 aa, synthetic construct
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCATYNFNKFYFGSGTKLNVKP

SEQ ID NO: 312 - DNA, 405 bp, synthetic construct
  source 1..405: mol_type = other DNA
atgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgccagcaccacttttaagacgggacgggcaattgaaaaactgttttttggcagtggaacccagctctctgtcttg

SEQ ID NO: 313 - DNA, 405 bp, synthetic construct
  source 1..405: mol_type = other DNA
atgggaccaaggctgctgttctgggcactgctgtgcctgctgggaaccggacctgtggaggccggcgtgacccagtctccaacacacctgatcaagaccaggggacagcaggccacactgaggtgtagccccatctccggccacacaagcgtgtactggtatcagcaggccctgggactgggactgcagttcctgctgtggtacgacgagggcgaggagaggaaccgcggcaatttcccacctcggttcagcggccggcagtttcccaactacagctccgagctgaacgtgaatgccctggagctggaggacagcgccctgtatctgtgcgcctccaccacattcaagaccggcagggccatcgagaagctgttctttggctctggcacccagctgagcgtgctg

SEQ ID NO: 314 - protein, 135 aa, synthetic construct
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASTTFKTGRAIEKLFFGSGTQLSVL

SEQ ID NO: 315 - protein, 266 aa, synthetic construct
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCATYNFNKFYFGSGTKLNVKPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 316 - protein, 308 aa, synthetic construct
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASTTFKTGRAIEKLFFGSGTQLSVLKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 317 - protein, 9 aa, synthetic construct
VVGADGVGK

SEQ ID NO: 318 - protein, 10 aa, synthetic construct
VVVGADGVGK

SEQ ID NO: 319 - protein, 6 aa, synthetic construct
TSGFNG

SEQ ID NO: 320 - protein, 6 aa, synthetic construct
NVLDGL

SEQ ID NO: 321 - protein, 12 aa, synthetic construct
AVRDRGGSYIPT

SEQ ID NO: 322 - protein, 5 aa, synthetic construct
MNHEY

SEQ ID NO: 323 - protein, 6 aa, synthetic construct
SMNVEV

SEQ ID NO: 324 - protein, 13 aa, synthetic construct
ASSSRGHSGTEAF

SEQ ID NO: 325 - DNA, 384 bp, synthetic construct
  source 1..384: mol_type = other DNA
atgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagatcgaggaggaagctacatacctacatttggaagaggaaccagccttattgttcatccg

SEQ ID NO: 326 - DNA, 384 bp, synthetic construct
  source 1..384: mol_type = other DNA
atgtggggcgtgtttctgctgtacgtgtctatgaagatgggcggcaccacaggccagaacatcgaccagcctaccgagatgaccgccacagagggcgccatcgtgcagatcaactgcacctaccagacatctggcttcaatggcctgttttggtatcagcagcacgccggcgaggccccaacattcctgtcctataatgtgctggatggcctggaggagaagggcaggttctctagctttctgtcccgctctaagggctacagctatctgctgctgaaggagctgcagatgaaggacagcgcctcctacctgtgcgccgtgcgggatagaggaggctcctatatccctacctttggccggggcacatctctgatcgtgcaccca

SEQ ID NO: 327 - protein, 128 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRDRGGSYIPTFGRGTSLIVHP

SEQ ID NO: 328 - DNA, 399 bp, synthetic construct
  source 1..399: mol_type = other DNA
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagttccagggggcattcgggcactgaagctttctttggacaaggcaccagactcacagttgta

SEQ ID NO: 329 - DNA, 399 bp, synthetic construct
  source 1..399: mol_type = other DNA
atgggaccacagctgctgggatacgtggtgctgtgcctgctgggagcaggaccactggaggcacaggtgacccagaacccacggtatctgatcaccgtgacaggcaagaagctgaccgtgacatgttctcagaacatgaatcacgagtacatgagctggtataggcaggaccctggactgggactgagacagatctactatagcatgaatgtggaggtgaccgacaagggcgatgtgcccgagggctacaaggtgtccaggaaggagaagcgcaacttccctctgatcctggagtccccatctcccaatcagaccagcctgtatttttgcgccagctcctctaggggacactccggaacagaggccttctttggccagggcaccaggctgacagtggtg

SEQ ID NO: 330 - protein, 133 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSRGHSGTEAFFGQGTRLTVV

SEQ ID NO: 331 - protein, 265 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRDRGGSYIPTFGRGTSLIVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 332 - protein, 306 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSRGHSGTEAFFGQGTRLTVVKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 333 - protein, 9 aa, synthetic construct
VVGADGVGK

SEQ ID NO: 334 - protein, 10 aa, synthetic construct
VVVGADGVGK

SEQ ID NO: 335 - protein, 5 aa, synthetic construct
TSINN

SEQ ID NO: 336 - protein, 7 aa, synthetic construct
IRSNERE

SEQ ID NO: 337 - protein, 11 aa, synthetic construct
AGLYSSASKII

SEQ ID NO: 338 - protein, 5 aa, synthetic construct
MNHEY

SEQ ID NO: 339 - protein, 6 aa, synthetic construct
SMNVEV

SEQ ID NO: 340 - protein, 13 aa, synthetic construct
ASSSRGHSGTEAF

SEQ ID NO: 341 - DNA, 393 bp, synthetic construct
  source 1..393: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctgggctgtacagcagtgcttccaagataatctttggatcagggaccagactcagcatccggcca

SEQ ID NO: 342 - DNA, 393 bp, synthetic construct
  source 1..393: mol_type = other DNA
atggagacactgctgggcgtgtccctggtcatcctgtggctgcagctggccagagtgaacagccagcagggagaggaggaccctcaggccctgagcatccaggagggcgagaacgccaccatgaattgctcttacaagacaagcatcaacaatctgcagtggtataggcagaactccggccgcggactggtgcacctgatcctgatcaggtctaatgagcgcgagaagcacagcggccggctgagagtgaccctggacacaagcaagaagtctagctccctgctgatcaccgcctccagagcagcagatacagcctcttacttctgtgccggcctgtattctagcgcctccaagatcatctttggcagcggcacccggctgtccatcagaccc

SEQ ID NO: 343 - protein, 131 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCAGLYSSASKIIFGSGTRLSIRP

SEQ ID NO: 344 - DNA, 399 bp, synthetic construct; identical to SEQ ID NO: 328
  source 1..399: mol_type = other DNA

SEQ ID NO: 345 - DNA, 399 bp, synthetic construct
  source 1..399: mol_type = other DNA
atgggcccacagctgctgggctacgtggtgctgtgcctgctgggagcaggaccactggaggcacaggtgacccagaaccccaggtatctgatcaccgtgacaggcaagaagctgaccgtgacatgtagccagaacatgaatcacgagtacatgtcctggtataggcaggaccccggactgggactgagacagatctactattccatgaatgtggaggtgaccgacaagggcgatgtgcctgagggctacaaggtgtctaggaaggagaagcgcaacttcccactgatcctggagtccccatctcccaatcagacctccctgtatttttgcgccagctcctctaggggccactctggcacagaggccttctttggccagggcaccaggctgacagtggtg

SEQ ID NO: 346 - protein, 133 aa, synthetic construct; identical to SEQ ID NO: 330

SEQ ID NO: 347 - protein, 268 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCAGLYSSASKIIFGSGTRLSIRPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 348 - protein, 306 aa, synthetic construct; identical to SEQ ID NO: 332

SEQ ID NO: 349 - protein, 9 aa, synthetic construct
VVGADGVGK

SEQ ID NO: 350 - protein, 10 aa, synthetic construct
VVVGADGVGK

SEQ ID NO: 351 - protein, 7 aa, synthetic construct
NIATNDY

SEQ ID NO: 352 - protein, 5 aa, synthetic construct
GYKTK

SEQ ID NO: 353 - protein, 10 aa, synthetic construct
LANTGGFKTI

SEQ ID NO: 354 - protein, 5 aa, synthetic construct
SGHVS

SEQ ID NO: 355 - protein, 6 aa, synthetic construct
FQNEAQ

SEQ ID NO: 356 - protein, 10 aa, synthetic construct
ATYKVGDEQF

SEQ ID NO: 357 - DNA, 378 bp, synthetic construct
  source 1..378: mol_type = other DNA
atgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgctaatactggaggcttcaaaactatctttggagcaggaacaagactatttgttaaagca

SEQ ID NO: 358 - DNA, 378 bp, synthetic construct
  source 1..378: mol_type = other DNA
atgaggcaggtggcacgcgtgatcgtgtttctgaccctgagcacactgtccctggccaagaccacacagcctatctctatggacagctacgagggccaggaggtgaacatcacctgctctcacaacaatatcgccaccaatgattacatcacatggtatcagcagttccccagccagggccctcggtttatcatccagggctataagaccaaggtgacaaacgaggtggccagcctgttcatccctgccgacaggaagtctagcaccctgtccctgccacgcgtgagcctgtccgatacagccgtgtactattgtctggccaataccggcggcttcaagacaatctttggcgccggcaccagactgtttgtgaaggcc

SEQ ID NO: 359 - protein, 126 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLANTGGFKTIFGAGTRLFVKA

SEQ ID NO: 360 - DNA, 393 bp, synthetic construct
  source 1..393: mol_type = other DNA
atgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccacctataaggtcggggatgagcagttcttcgggccagggacacggctcaccgtgcta

SEQ ID NO: 361 - DNA, 393 bp, synthetic construct
  source 1..393: mol_type = other DNA
atgggaaccaggctgctgtgctgggtggtgctgggcttcctgggaaccgaccacacaggagcaggcgtgtcccagtctccaaggtacaaggtggcaaagaggggacaggacgtggccctgagatgtgatcctatctccggccacgtgtctctgttttggtaccagcaggccctgggacagggacctgagttcctgacctattttcagaacgaggcacagctggacaagagcggactgccatccgatcggttctttgcagagagaccagagggcagcgtgtccaccctgaagatccagaggacacagcaggaggactccgccgtgtacctgtgcgccacatataaagtgggcgatgagcagttctttggcccaggcacccggctgacagtgctg

SEQ ID NO: 362 - protein, 131 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCATYKVGDEQFFGPGTRLTVL

SEQ ID NO: 363 - protein, 263 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLANTGGFKTIFGAGTRLFVKADIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 364 - protein, 304 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCATYKVGDEQFFGPGTRLTVLKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 365 - protein, 9 aa, synthetic construct
VVGADGVGK

SEQ ID NO: 366 - protein, 10 aa, synthetic construct
VVVGADGVGK

SEQ ID NO: 367 - protein, 6 aa, synthetic construct
DSASNY

SEQ ID NO: 368 - protein, 7 aa, synthetic construct
IRSNVGE

SEQ ID NO: 369 - protein, 9 aa, synthetic construct
AETGFQKLV

SEQ ID NO: 370 - protein, 5 aa, synthetic construct
MDHEN

SEQ ID NO: 371 - protein, 6 aa, synthetic construct
SYDVKM

SEQ ID NO: 372 - protein, 14 aa, synthetic construct
ASSDWLAGAKDEQY

SEQ ID NO: 373 - DNA, 387 bp, synthetic construct
  source 1..387: mol_type = other DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagaaacaggctttcagaaacttgtatttggaactggcacccgacttctggtcagtcca

SEQ ID NO: 374 - DNA, 387 bp, synthetic construct
  source 1..387: mol_type = other DNA
atgacatctatccgcgccgtgttcatctttctgtggctgcagctggacctggtgaacggcgagaatgtggagcagcacccaagcaccctgtccgtgcaggagggcgacagcgccgtgatcaagtgcacatactctgatagcgcctccaactactttccctggtataagcaggagctgggcaagcggcctcagctgatcatcgacatcagatccaacgtgggcgagaagaaggatcagcggatcgccgtgaccctgaataagacagccaagcacttcagcctgcacatcaccgagacacagcccgaggattccgccgtgtatttttgtgccgagaccggcttccagaagctggtgtttggcaccggcacaagactgctggtgtcccct

SEQ ID NO: 375 - protein, 129 aa, synthetic construct
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAETGFQKLVFGTGTRLLVSP

SEQ ID NO: 376 - DNA, 402 bp, synthetic construct
  source 1..402: mol_type = other DNA
atgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtgactggctagcgggagcgaaggacgagcagtacttcgggccgggcaccaggctcacggtcaca

SEQ ID NO: 377 - DNA, 402 bp, synthetic construct
  source 1..402: mol_type = other DNA
atgggcatccggctgctgtgcagagtggccttctgttttctggccgtgggcctggtggacgtgaaggtgacccagagctcccggtacctggtgaagagaacaggcgagaaggtgttcctggagtgcgtgcaggacatggatcacgagaacatgttttggtataggcaggaccccggactgggactgagactgatctacttcagctatgacgtgaagatgaaggagaagggcgacatcccagagggctacagcgtgtccagggagaagaaggagcggttcagcctgatcctggagtctgccagcaccaatcagacaagcatgtacctgtgcgcctctagcgactggctggccggagcaaaggatgagcagtatttcggcccaggcaccaggctgaccgtgaca

SEQ ID NO: 378 - protein, 134 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSDWLAGAKDEQYFGPGTRLTVT

SEQ ID NO: 379 - protein, 266 aa, synthetic construct
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAETGFQKLVFGTGTRLLVSPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 380 - protein, 307 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSDWLAGAKDEQYFGPGTRLTVTKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 381 - protein, 9 aa, synthetic construct
VVGADGVGK

SEQ ID NO: 382 - protein, 10 aa, synthetic construct
VVVGADGVGK

SEQ ID NO: 383 - protein, 5 aa, synthetic construct
TSINN

SEQ ID NO: 384 - protein, 7 aa, synthetic construct
IRSNERE

SEQ ID NO: 385 - protein, 10 aa, synthetic construct
ATDPLDYKLS

SEQ ID NO: 386 - protein, 5 aa, synthetic construct
MNHEY

SEQ ID NO: 387 - protein, 6 aa, synthetic construct
SMNVEV

SEQ ID NO: 388 - protein, 11 aa, synthetic construct
ASSLVASNEQF

SEQ ID NO: 389 - DNA, 390 bp, synthetic construct
  source 1..390: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggaccccttagactacaagctcagctttggagccggaaccacagtaactgtaagagca

SEQ ID NO: 390 - DNA, 390 bp, synthetic construct
  source 1..390: mol_type = other DNA
atggagaccctgctgggcgtgtctctggtcatcctgtggctgcagctggccagagtgaactctcagcagggagaggaggaccctcaggccctgagcatccaggagggcgagaacgccaccatgaattgctcttacaagacaagcatcaacaatctgcagtggtatcggcagaactccggcagaggcctggtgcacctgatcctgatcaggagcaatgagcgcgagaagcactccggccggctgagagtgaccctggacacatctaagaagtcctctagcctgctgatcaccgcctctagggcagcagatacagccagctacttctgtgccaccgacccactggattataagctgtcctttggcgccggcaccacagtgaccgtgcgcgcc

SEQ ID NO: 391 - protein, 130 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDPLDYKLSFGAGTTVTVRA

SEQ ID NO: 392 - DNA, 393 bp, synthetic construct
  source 1..393: mol_type = other DNA
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttggtggctagcaatgagcagttcttcgggccagggacacggctcaccgtgcta

SEQ ID NO: 393 - DNA, 393 bp, synthetic construct
  source 1..393: mol_type = other DNA
atgggcccacagctgctgggctacgtggtgctgtgcctgctgggagcaggaccactggaggcacaggtgacccagaatccccggtatctgatcaccgtgacaggcaagaagctgaccgtgacatgttcccagaacatgaatcacgagtacatgtcttggtataggcaggaccccggactgggactgaggcagatctactattctatgaacgtggaggtgacagacaagggcgatgtgcctgagggctacaaggtgagcaggaaggagaagcgcaacttcccactgatcctggagtccccatctcccaatcagaccagcctgtatttttgcgccagctccctggtggcctccaacgagcagttctttggccctggcacccggctgacagtgctg

SEQ ID NO: 394 - protein, 131 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLVASNEQFFGPGTRLTVL

SEQ ID NO: 395 - protein, 267 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDPLDYKLSFGAGTTVTVRADIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 396 - protein, 304 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLVASNEQFFGPGTRLTVLKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 397 - protein, 9 aa, synthetic construct
VVGAVGVGK

SEQ ID NO: 398 - protein, 10 aa, synthetic construct
VVVGAVGVGK

SEQ ID NO: 399 - protein, 7 aa, synthetic construct
TSESDYY

SEQ ID NO: 400 - protein, 8 aa, synthetic construct
QEAYKQQN

SEQ ID NO: 401 - protein, 10 aa, synthetic construct
ACQGGSEKLV

SEQ ID NO: 402 - protein, 5 aa, synthetic construct
SGHNT

SEQ ID NO: 403 - protein, 6 aa, synthetic construct
YYREEE

SEQ ID NO: 404 - protein, 13 aa, synthetic construct
ASSLGLLLYNEQF

SEQ ID NO: 405 - DNA, 399 bp, synthetic construct
  source 1..399: mol_type = other DNA
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttgtcagggcggatctgaaaagctggtctttggaaagggaacgaaactgacagtaaaccca

SEQ ID NO: 406 - DNA, 399 bp, synthetic construct
  source 1..399: mol_type = other DNA
atggcatgcccaggcttcctgtgggcactggtcatcagcacctgtctggagttttctatggcccagaccgtgacacagagccagccagagatgtccgtgcaggaggcagagaccgtgacactgtcctgtacctacgacacaagcgagtccgattactatctgttctggtataagcagcctccatctcgccagatgatcctggtcatccggcaggaggcctacaagcagcagaacgccaccgagaatcggttctctgtgaattttcagaaggccgccaagtcttttagcctgaagatctccgactctcagctgggcgatgccgccatgtatttctgcgcatgtcagggaggcagcgagaagctggtgtttggcaagggcaccaagctgacagtgaaccct

SEQ ID NO: 407 - protein, 133 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCACQGGSEKLVFGKGTKLTVNP

SEQ ID NO: 408 - DNA, 399 bp, synthetic construct
  source 1..399: mol_type = other DNA
atgggccctgggctcctctgctgggtgctgctttgtctcctgggagcaggctcagtggagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagcttgggactcctcctctacaatgagcagttcttcgggccagggacacggctcaccgtgcta

SEQ ID NO: 409 - DNA, 399 bp, synthetic construct
  source 1..399: mol_type = other DNA
atgggaccaggactgctgtgctgggtgctgctgtgcctgctgggagcaggcagcgtggagaccggcgtgacacagtcccctacccacctgatcaagacaagaggccagcaggtgaccctgaggtgcagctcccagtctggccacaatacagtgagctggtaccagcaggccctgggacagggacctcagttcatctttcagtactatagggaggaggagaacggccgcggcaatttcccccctcggtttagcggcctgcagttcccaaactattctagcgagctgaacgtgaatgccctggagctggacgattccgccctgtacctgtgcgcctcctctctgggcctgctgctgtataacgagcagttctttggccccggcaccagactgacagtgctg

SEQ ID NO: 410 - protein, 133 aa, synthetic construct
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSLGLLLYNEQFFGPGTRLTVL

SEQ ID NO: 411 - protein, 270 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCACQGGSEKLVFGKGTKLTVNPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 412 - protein, 306 aa, synthetic construct
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSLGLLLYNEQFFGPGTRLTVLKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 413 - protein, 9 aa, synthetic construct
VVGAVGVGK

SEQ ID NO: 414 - protein, 10 aa, synthetic construct
VVVGAVGVGK

SEQ ID NO: 415 - protein, 5 aa, synthetic construct
TSINN

SEQ ID NO: 416 - protein, 7 aa, synthetic construct
IRSNERE

SEQ ID NO: 417 - protein, 12 aa, synthetic construct
ATDAQTGANNLF

SEQ ID NO: 418 - protein, 5 aa, synthetic construct
SGHAT

SEQ ID NO: 419 - protein, 6 aa, synthetic construct
FQNNGV

SEQ ID NO: 420 - protein, 12 aa, synthetic construct
ASSLGDSYEQYF

SEQ ID NO: 421 - DNA, 396 bp, synthetic construct
  source 1..396: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgctcaaactggggcaaacaacctcttctttgggactggaacgagactcaccgttattccc

SEQ ID NO: 422 - DNA, 396 bp, synthetic construct
  source 1..396: mol_type = other DNA
atggagacactgctgggcgtgtctctggtcatcctgtggctgcagctggccagagtgaatagccagcagggagaggaggacccccaggccctgtccatccaggagggcgagaacgccaccatgaattgcagctacaagacatccatcaacaatctgcagtggtatcggcagaactctggcagaggcctggtgcacctgatcctgatccggtccaatgagagagagaagcactctggccggctgagagtgaccctggatacatccaagaagtcctctagcctgctgatcaccgccagccgggcagcagacacagcctcctatttttgtgccaccgatgcccagacaggcgccaacaatctgttctttggcaccggcacaagactgaccgtgatccct

SEQ ID NO: 423 - protein, 132 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAQTGANNLFFGTGTRLTVIP

SEQ ID NO: 424 - DNA, 345 bp, synthetic construct
  source 1..345: mol_type = other DNA
atgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagctta

SEQ ID NO: 425 - DNA, 345 bp, synthetic construct
  source 1..345: mol_type = other DNA
atgggcaccaggctgctgtgctgggccgccctgtgcctgctgggagcagagctgacagaggcaggagtggcccagagccccaggtacaagatcatcgagaagcgccagtccgtggccttctggtgcaaccctatctctggccacgccaccctgtactggtatcagcagatcctgggccagggcccaaagctgctgatccagttccagaacaatggcgtggtggacgattctcagctgcccaaggacaggtttagcgccgagcgcctgaagggcgtggattctaccctgaagatccagccagcaaagctggaggacagcgccgtgtacctgtgcgccagctccctg

SEQ ID NO: 426 - protein, 115 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSL

SEQ ID NO: 427 - protein, 269 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAQTGANNLFFGTGTRLTVIPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 428 - protein, 288 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 429 - protein, 9 aa, synthetic construct
VVGAVGVGK

SEQ ID NO: 430 - protein, 10 aa, synthetic construct
VVVGAVGVGK

SEQ ID NO: 431-531 - skipped (101 entries)

SEQ ID NO: 532 - protein, 6 aa, synthetic construct
NSMFDY

SEQ ID NO: 533 - protein, 7 aa, synthetic construct
ISSIKDK

SEQ ID NO: 534 - protein, 13 aa, synthetic construct
AANAGGTSYGKLT

SEQ ID NO: 535 - protein, 5 aa, synthetic construct
SNHLY

SEQ ID NO: 536 - protein, 6 aa, synthetic construct
FYNNEI

SEQ ID NO: 537 - protein, 12 aa, synthetic construct
ASSEWGSTGELF

SEQ ID NO: 538 - DNA, 421 bp, synthetic construct
  source 1..421: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaaatgctggtggtactagctatggaaagctgacatttggacaagggaccatcttgactgtccatccaa

SEQ ID NO: 539 - protein, 140 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAANAGGTSYGKLTFGQGTILTVHP

SEQ ID NO: 540 - DNA, 400 bp, synthetic construct
  source 1..400: mol_type = other DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaatggggaagcaccggggagctgttttttggagaaggctctaggctgaccgtactgg

SEQ ID NO: 541 - protein, 133 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEWGSTGELFFGEGSRLTVL

SEQ ID NO: 542 - protein, 277 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAANAGGTSYGKLTFGQGTILTVHPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 543 - protein, 306 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEWGSTGELFFGEGSRLTVLKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 544 - protein, 9 aa, synthetic construct
VVGAVGVGK

SEQ ID NO: 545 - protein, 5 aa, synthetic construct
TTSDR

SEQ ID NO: 546 - protein, 7 aa, synthetic construct
LLSNGAV

SEQ ID NO: 547 - protein, 10 aa, synthetic construct
AVDIIGGKST

SEQ ID NO: 548 - protein, 5 aa, synthetic construct
SNHLY

SEQ ID NO: 549 - protein, 6 aa, synthetic construct
FYNNEI

SEQ ID NO: 550 - protein, 12 aa, synthetic construct
ASSEWGSTGELF

SEQ ID NO: 551 - DNA, 385 bp, synthetic construct
  source 1..385: mol_type = other DNA
atgaagaagctactagcaatgattctgtggcttcaactagaccggttaagtggagagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgtgccgtggacatcatcggaggcaaatcaacctttggggatgggactacgctcactgtgaagccaa

SEQ ID NO: 552 - protein, 128 aa, synthetic construct
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDIIGGKSTFGDGTTLTVKP

SEQ ID NO: 553 - DNA, 400 bp, synthetic construct; identical to SEQ ID NO: 540
  source 1..400: mol_type = other DNA

SEQ ID NO: 554 - protein, 133 aa, synthetic construct; identical to SEQ ID NO: 541

SEQ ID NO: 555 - protein, 265 aa, synthetic construct
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDIIGGKSTFGDGTTLTVKPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 556 - protein, 306 aa, synthetic construct; identical to SEQ ID NO: 543

SEQ ID NO: 557 - protein, 9 aa, synthetic construct
VVGAVGVGK

SEQ ID NO: 558 - protein, 6 aa, synthetic construct
NSMFDY

SEQ ID NO: 559 - protein, 7 aa, synthetic construct
ISSIKDK

SEQ ID NO: 560 - protein, 14 aa, synthetic construct
AASAVGQEYGNKLV

SEQ ID NO: 561 - protein, 5 aa, synthetic construct
SEHNR

SEQ ID NO: 562 - protein, 6 aa, synthetic construct
FQNEAQ

SEQ ID NO: 563 - protein, 11 aa, synthetic construct
ASSEYTMGTQY

SEQ ID NO: 564 - DNA, 424 bp, synthetic construct
  source 1..424: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgcagtaggtcaggaatatggaaacaagctggtctttggcgcaggaaccattctgagagtcaagtcct

SEQ ID NO: 565 - protein, 141 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASAVGQEYGNKLVFGAGTILRVKS

SEQ ID NO: 566 - DNA, 397 bp, synthetic construct
  source 1..397: mol_type = other DNA
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagtgaatatactatggggacccagtacttcgggccaggcacgcggctcctggtgctcg

SEQ ID NO: 567 - protein, 132 aa, synthetic construct
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSEYTMGTQYFGPGTRLLVL

SEQ ID NO: 568 - protein, 278 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASAVGQEYGNKLVFGAGTILRVKSDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 569 - protein, 305 aa, synthetic construct
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSEYTMGTQYFGPGTRLLVLKDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 570-599 - skipped (30 entries)

SEQ ID NO: 600 - protein, 9 aa, synthetic construct
VVGAVGVGK

SEQ ID NO: 601 - protein, 253 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 602 - protein, 253 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGGGSGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 603 - protein, 122 aa, synthetic construct [sequence omitted]

SEQ ID NO: 604 - protein, 107 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 605 - protein, 5 aa, synthetic construct
GYTMN

SEQ ID NO: 606 - protein, 17 aa, synthetic construct
LINPYKGVSTYNQKFKD

SEQ ID NO: 607 - protein, 13 aa, synthetic construct
SGYYGDSDWYFDV

SEQ ID NO: 608 - protein, 11 aa, synthetic construct
RASQDIRNYLN

SEQ ID NO: 609 - protein, 7 aa, synthetic construct
YTSRLES

SEQ ID NO: 610 - protein, 9 aa, synthetic construct
QQGNTLPWT

SEQ ID NO: 611 - protein, 25 aa, synthetic construct
GGGGSGGGGSGGGGSGGGGSGGGGS

SEQ ID NO: 612 - protein, 253 aa, synthetic construct
DQQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 613 - protein, 253 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 614 - protein, 255 aa, synthetic construct
AQDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 615 - protein, 124 aa, synthetic construct
EVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSS

SEQ ID NO: 616 - protein, 5 aa, synthetic construct
GYSMA

SEQ ID NO: 617 - protein, 17 aa, synthetic construct
AIVWSDGNTYYEDFVKG

SEQ ID NO: 618 - protein, 15 aa, synthetic construct
KIRPYIFKIAGQYDY

SEQ ID NO: 619 - protein, 113 aa, synthetic construct
LAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSD

SEQ ID NO: 620 - protein, 113 aa, synthetic construct
AGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVV

SEQ ID NO: 621 - protein, 111 aa, synthetic construct
QQKEVEQNSGPLSVPEGAIASLNCTYSDRGSQSFFWYRQYSGKSPELIMSIYSNGDKEDGRFTAQLNKASQYVSLLIRDSQPSDSATYLCAGGGGADGLTFGKGTHLIIQP

SEQ ID NO: 622 - protein, 112 aa, synthetic construct
GITQSPKYLFRKEGQNVTLSCEQNLNHDAMYWYRQDPGQGLRLIYYSQIVNDFQKGDIAEGYSVSREKKESFPLTVTSAQKNPTAFYLCASSQGLAGAGELFFGEGSRLTVL

SEQ ID NO: 623 - protein, 125 aa, synthetic construct
QVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSS

SEQ ID NO: 624 - protein, 253 aa, synthetic construct
EVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSGGGGSGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKR

SEQ ID NO: 625 - protein, 125 aa, synthetic construct
EVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSS

SEQ ID NO: 626 - protein, 113 aa, synthetic construct
DVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKR

SEQ ID NO: 627 - protein, 18 aa, synthetic construct
TNFSLLKQAGDVEENPGP

SEQ ID NO: 628 - protein, 33 aa, synthetic construct
GAAPAAAPAKQEAAAPAPAAKAEAPAAAPAAKA

SEQ ID NO: 629 - protein, 7 aa, synthetic construct
SGGGGSG

SEQ ID NO: 630 - protein, 17 aa, synthetic construct
SSGGGGSGGGGSGGGGS

SEQ ID NO: 631 - protein, 31 aa, synthetic construct
AQLEKELQALEKENAQLEWELQALEKELAQK

SEQ ID NO: 632 - protein, 31 aa, synthetic construct
AQLKKKLQALKKKNAQLKWKLQALKKKLAQK

SEQ ID NO: 633 - protein, 556 aa, synthetic construct
METDTLLLWVLLLWVPGSTGDLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMETDTLLLWVLLLWVPGSTGDDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGSGGLNDIFEAQKIEWHE

SEQ ID NO: 634 - protein, 471 aa, synthetic construct
METLLGVSLVILWLQLARVNSQQKEVEQNSGPLSVPEGAIASLNCTYSDRGSQSFFWYRQYSGKSPELIMSIYSNGDKEDGRFTAQLNKASQYVSLLIRDSQPSDSATYLCAGGGGADGLTFGKGTHLIIQPYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGHHHHHHHHH

SEQ ID NO: 635 - protein, 264 aa, synthetic construct
MGTRLLCWAALCLLGAELTEAGITQSPKYLFRKEGQNVTLSCEQNLNHDAMYWYRQDPGQGLRLIYYSQIVNDFQKGDIAEGYSVSREKKESFPLTVTSAQKNPTAFYLCASSQGLAGAGELFFGEGSRLTVLEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADC

SEQ ID NO: 636 - protein, 9 aa, synthetic construct
HHHHHHHHH

SEQ ID NO: 637 - protein, 423 aa, synthetic construct
MWWRLWWLLLLLLLLWPMVWAAQQKEVEQNSGPLSVPEGAIASLNCTYSDRGSQSFFWYRQYSGKSPELIMSIYSNGDKEDGRFTAQLNKASQYVSLLIRDSQPSDSATYLCAGGGGADGLTFGKGTHLIICPGSDYEFLKSWTVEDLQKRLLALDPMMEQEIEEIRQKYQSKRQPILDAIEAKGSGHHHHHHHHHHRIRRSGSGATNFSLLKQAGDVEENPGPMWWRLWWLLLLLLLLWPMVWAAGITQSPKYLFRKEGQNVTLSCEQNLNHDAMYWYRQDPGQGLRLIYYSQIVNDFQKGDIAEGYSVSREKKESFPLTVTSAQKNPTAFYLCASSQGLAGAGELFFGEGSRLTVLEGSDYEFLKSWTVEDLQKRLLALDPMMEQEIEEIRQKYQCKRQPILDAIEAKGSGSAWSHPQFEK

SEQ ID NO: 638 - protein, 49 aa, synthetic construct
DYEFLKSWTVEDLQKRLLALDPMMEQEIEEIRQKYQCKRQPILDAIEAK

SEQ ID NO: 639 - protein, 484 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSHHHHHHHHHH

SEQ ID NO: 640 - protein, 526 aa, synthetic construct
MMGVKVLFALICIAVAEADDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSGGGGSDKTHTCPPCPAPELLGGPSVFLFPPKPKDTLEASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNAYTQKSLSLSPGKGGGGSGLNDIFEAQKIEWHE

SEQ ID NO: 641 - protein, 826 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 642 - protein, 807 aa, synthetic construct
LAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 643 - protein, 676 aa, synthetic construct
MMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGSDYEFLKSWTVEDLQKRLLALDPMMEQEIEEIRQKYQCKRQPILDAIEAKGSGHHHHHHHHHHRIRRSGSGATNFSLLKQAGDVEENPGPMMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVCPDGSDYEFLKSWTVEDLQKRLLALDPMMEQEIEEIRQKYQSKRQPILDAIEAKGSGSAWSHPQFEK

SEQ ID NO: 644 - protein, 658 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGSDYEFLKSWTVEDLQKRLLALDPMMEQEIEEIRQKYQCKRQPILDAIEAKGSGHHHHHHHHHHRIRRSGSGATNFSLLKQAGDVEENPGPMMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVCPDGSDYEFLKSWTVEDLQKRLLALDPMMEQEIEEIRQKYQSKRQPILDAIEAKGSGSAWSHPQFEK

SEQ ID NO: 645 - protein, 495 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSLPETGGHHHHHHHHHH

SEQ ID NO: 646 - protein, 783 aa, synthetic construct
MMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSGGGGSDKTHTCPPCPAPELLGGPSVFLFPPKPKDTLEASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNAYTQKSLSLSPGKGGGGSGLNDIFEAQKIEWHE

SEQ ID NO: 647 - protein, 825 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGQTNYGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 648 - protein, 770 aa, synthetic construct
LAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGQTNYGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADC

SEQ ID NO: 649 - protein, 495 aa, synthetic construct; identical to SEQ ID NO: 645

SEQ ID NO: 650 - protein, 782 aa, synthetic construct
MMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGQTNYGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSGGGGSDKTHTCPPCPAPELLGGPSVFLFPPKPKDTLEASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNAYTQKSLSLSPGKGGGGSGLNDIFEAQKIEWHE

SEQ ID NO: 651 - protein, 821 aa, synthetic construct
MMGVKVLFALICIAVAEAQKEVEQNSGPLSVPEGAIASLNCTYSDRGSQSFFWYRQYSGKSPELIMFIYSNGDKEDGRFTAQLNKASQYVSLLIRDSQPSDSATYLCAAAMDSSYKLIFGSGTRLLVRPDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGITQSPRYKITETGRQVTLMCHQTWSHSYMFWYRQDLGHGLRLIYYSAAADITDKGEVPDGYVVSRSKTENFPLTLESATRSQTSVYFCASSDPGTEAFFGQGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 652 - protein, 803 aa, synthetic construct
QKEVEQNSGPLSVPEGAIASLNCTYSDRGSQSFFWYRQYSGKSPELIMFIYSNGDKEDGRFTAQLNKASQYVSLLIRDSQPSDSATYLCAAAMDSSYKLIFGSGTRLLVRPDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGITQSPRYKITETGRQVTLMCHQTWSHSYMFWYRQDLGHGLRLIYYSAAADITDKGEVPDGYVVSRSKTENFPLTLESATRSQTSVYFCASSDPGTEAFFGQGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 653 - protein, 21 aa, synthetic construct
METDTLLLWVLLLWVPGSTGD

SEQ ID NO: 654 - protein, 21 aa, synthetic construct
METLLGVSLVILWLQLARVNS

SEQ ID NO: 655 - protein, 21 aa, synthetic construct
MGTRLLCWAALCLLGAELTEA

SEQ ID NO: 656-665 - skipped (10 entries)

SEQ ID NO: 666 - protein, 22 aa, synthetic construct
MWWRLWWLLLLLLLLWPMVWAA

SEQ ID NO: 667 - protein, 19 aa, synthetic construct
MMGVKVLFALICIAVAEAD

SEQ ID NO: 668 - protein, 18 aa, synthetic construct
MMGVKVLFALICIAVAEA

SEQ ID NO: 669 - protein, 245 aa, synthetic construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 670 - protein, 250 aa, synthetic construct
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 671 - protein, 119 aa, synthetic construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 672 - protein, 106 aa, synthetic construct
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 673 - protein, 5 aa, synthetic construct
RYTMH

SEQ ID NO: 674 - protein, 17 aa, synthetic construct
YINPSRGYTNYNQKFKD

SEQ ID NO: 675 - protein, 10 aa, synthetic construct
YYDDHYCLDY

SEQ ID NO: 676 - protein, 10 aa, synthetic construct
RASSSVSYMN

SEQ ID NO: 677 - protein, 7 aa, synthetic construct
DTSKVAS

SEQ ID NO: 678 - protein, 9 aa, synthetic construct
QQWSSNPLT

SEQ ID NO: 679 - protein, 117 aa, synthetic construct
KLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 680 - protein, 208 aa, synthetic construct
LAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSC

SEQ ID NO: 681 - protein, 95 aa, synthetic construct
YIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSC

SEQ ID NO: 682 - protein, 24 aa, synthetic construct
GGGGSGGGGSGGGGSGGGGSGGGS

SEQ ID NO: 683 - protein, 5 aa, synthetic construct
GGGGS

SEQ ID NO: 684 - protein, 244 aa, synthetic construct
AGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADC

SEQ ID NO: 685 - protein, 131 aa, synthetic construct
EDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADC

SEQ ID NO: 686 - protein, 36 aa, synthetic construct
GGGGSGLNDIFEAQKIEWHEGGGGSGGGGSLPETGG

SEQ ID NO: 687 - protein, 51 aa, synthetic construct
GSDYEFLKSWTVEDLQKRLLALDPMMEQEIEEIRQKYQCKRQPILDAIEAK

SEQ ID NO: 688 - protein, 40 aa, synthetic construct
GSGHHHHHHHHHHRIRRSGSGATNFSLLKQAGDVEENPGP

SEQ ID NO: 689 - protein, 67 aa, synthetic construct
CPDGSDYEFLKSWTVEDLQKRLLALDPMMEQEIEEIRQKYQSKRQPILDAIEAKGSGSAWSHPQFEK

SEQ ID NO: 690 - protein, 15 aa, synthetic construct
GGGGSGGGGSGGGGS

SEQ ID NO: 691 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 692 - protein, 227 aa, synthetic construct
DKTHTCPPCPAPELLGGPSVFLFPPKPKDTLEASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNAYTQKSLSLSPGK

SEQ ID NO: 693 - protein, 6 aa, synthetic construct
GGGGSD

SEQ ID NO: 694 - protein, 20 aa, synthetic construct
GGGGSGLNDIFEAQKIEWHE

SEQ ID NO: 695 - protein, 26 aa, synthetic construct
GGGGSGGGGSLPETGGHHHHHHHHHH

SEQ ID NO: 696 - protein, 43 aa, synthetic construct
GSGHHHHHHHHHHRIRRSGSGVKQTLNFDLLKLAGDVESNPGP

SEQ ID NO: 697 - protein, 244 aa, synthetic construct
DAGVTQSPTHLIKTRGQQVTLRCSPKSGHDTVSWYQQALGQGPQFIFQYYEEEERQRGNFPDRFSGHQFPNYSSELNVNALLLGDSALYLCASSLGQTNYGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADC

SEQ ID NO: 698 - protein, 207 aa, synthetic construct
QKEVEQNSGPLSVPEGAIASLNCTYSDRGSQSFFWYRQYSGKSPELIMFIYSNGDKEDGRFTAQLNKASQYVSLLIRDSQPSDSATYLCAAAMDSSYKLIFGSGTRLLVRPDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSC

SEQ ID NO: 699 - protein, 43 aa, synthetic construct; identical to SEQ ID NO: 696

SEQ ID NO: 700 - protein, 241 aa, synthetic construct
AGITQSPRYKITETGRQVTLMCHQTWSHSYMFWYRQDLGHGLRLIYYSAAADITDKGEVPDGYVVSRSKTENFPLTLESATRSQTSVYFCASSDPGTEAFFGQGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADC

SEQ ID NO: 701 - protein, 254 aa, synthetic construct
QVQLVQSGGGLVQPGGPLRLSCTASGFSFSNNYYMCWVRQAPGKGLEWVSCIYTNNIGSTWYASWAKGRFTISRDNSKNTLYLQMNSLRAEDTAVYYCARGIYSDSSDFIGNLWGQGTLVTVSSGGGGSGGGGSGGGGSGGGGSAIQMTQSPSSLSASVGDRVTITCQASQTIYNYLAWYQQKPGKVPELLIYKASTLASGVPSRFMGTGFGTDFTLTIDSLQPEDAATYYCQQYNTIINVDRTFGGGTKVEIK

SEQ ID NO: 702 - protein, 259 aa, synthetic construct
AIQMTQSPSSLSASVGDRVTITCQASQTIYNYLAWYQQKPGKVPELLIYKASTLASGVPSRFMGTGFGTDFTLTIDSLQPEDAATYYCQQYNTIINVDRTFGGGTKVEIKGGGGSGGGGSGGGGSGGGGSGGGGSQVQLVQSGGGLVQPGGPLRLSCTASGFSFSNNYYMCWVRQAPGKGLEWVSCIYTNNIGSTWYASWAKGRFTISRDNSKNTLYLQMNSLRAEDTAVYYCARGIYSDSSDFIGNLWGQGTLVTVSS

SEQ ID NO: 703 - protein, 124 aa, synthetic construct
QVQLVQSGGGLVQPGGPLRLSCTASGFSFSNNYYMCWVRQAPGKGLEWVSCIYTNNIGSTWYASWAKGRFTISRDNSKNTLYLQMNSLRAEDTAVYYCARGIYSDSSDFIGNLWGQGTLVTVSS

SEQ ID NO: 704 - protein, 110 aa, synthetic construct
AIQMTQSPSSLSASVGDRVTITCQASQTIYNYLAWYQQKPGKVPELLIYKASTLASGVPSRFMGTGFGTDFTLTIDSLQPEDAATYYCQQYNTIINVDRTFGGGTKVEIK

SEQ ID NO: 705 - protein, 6 aa, synthetic construct
NNYYMC

SEQ ID NO: 706 - protein, 18 aa, synthetic construct
CIYTNNIGSTWYASWAKG

SEQ ID NO: 707 - protein, 13 aa, synthetic construct
GIYSDSSDFIGNL

SEQ ID NO: 708 - protein, 11 aa, synthetic construct
QASQTIYNYLA

SEQ ID NO: 709 - protein, 7 aa, synthetic construct
KASTLAS

SEQ ID NO: 710 - protein, 12 aa, synthetic construct
QQYNTIINVDRT

SEQ ID NO: 711 - protein, 9 aa, synthetic construct
GFSFSNNYY

SEQ ID NO: 712 - protein, 9 aa, synthetic construct
IYTNNIGST

SEQ ID NO: 713 - protein, 15 aa, synthetic construct
ARGIYSDSSDFIGNL

SEQ ID NO: 714 - protein, 6 aa, synthetic construct
QTIYNY

SEQ ID NO: 715 - skipped

SEQ ID NO: 716 - protein, 12 aa, synthetic construct
QQYNTIINVDRT

SEQ ID NO: 717 - protein, 10 aa, synthetic construct
YYDDHYSLDY

SEQ ID NO: 718 - protein, 8 aa, synthetic construct
GYTFTRYT

SEQ ID NO: 719 - protein, 8 aa, synthetic construct
INPSRGYT

SEQ ID NO: 720 - protein, 12 aa, synthetic construct
ARYYDDHYCLDY

SEQ ID NO: 721 - protein, 12 aa, synthetic construct
ARYYDDHYSLDY

SEQ ID NO: 722 - protein, 5 aa, synthetic construct
SSVSY

SEQ ID NO: 723 - protein, 8 aa, synthetic construct
INPSRGYT

SEQ ID NO: 724 - protein, 9 aa, synthetic construct
QQWSSNPLT

SEQ ID NO: 725 - protein, 245 aa, synthetic construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 726 - protein, 119 aa, synthetic construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 727 - protein, 245 aa, synthetic construct; identical to SEQ ID NO: 669

SEQ ID NO: 728 - protein, 250 aa, synthetic construct
QIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 729 - protein, 8 aa, synthetic construct
GYSFTGYT

SEQ ID NO: 730 - protein, 8 aa, synthetic construct
INPYKGVS

SEQ ID NO: 731 - protein, 15 aa, synthetic construct
ARSGYYGDSDWYFDV

SEQ ID NO: 732 - protein, 6 aa, synthetic construct
QDIRNY

SEQ ID NO: 733 - skipped

SEQ ID NO: 734 - protein, 8 aa, synthetic construct
GRTYRGYS

SEQ ID NO: 735 - protein, 8 aa, synthetic construct
IVWSDGNT

SEQ ID NO: 736 - protein, 17 aa, synthetic construct
AAKIRPYIFKIAGQYDY

SEQ ID NO: 737 - protein, 5 aa, synthetic construct
GYVMH

SEQ ID NO: 738 - protein, 17 aa, synthetic construct
FINPYNDDIQSNERFRG

SEQ ID NO: 739 - protein, 15 aa, synthetic construct
GAGYNFDGAYRFEDF

SEQ ID NO: 740 - protein, 8 aa, synthetic construct
GYTFTGYV

SEQ ID NO: 741 - protein, 8 aa, synthetic construct
INPYNDDI

SEQ ID NO: 742 - protein, 17 aa, synthetic construct
ARGAGYNFDGAYRFEDF

SEQ ID NO: 743 - protein, 16 aa, synthetic construct
RSSQRLVHSNGNTYLH

SEQ ID NO: 744 - protein, 7 aa, synthetic construct
RVSNRFP

SEQ ID NO: 745 - protein, 9 aa, synthetic construct
SQSTHVPYT

SEQ ID NO: 746 - protein, 11 aa, synthetic construct
QRLVHSNGNTY

SEQ ID NO: 747 - skipped

SEQ ID NO: 748 - protein, 253 aa, synthetic construct
DVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGGGGSGGGGSGGGGSEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSS

SEQ ID NO: 749 - protein, 19 aa, synthetic construct
AAVRDYVGMPYYSGSAYEY

SEQ ID NO: 750 - protein, 17 aa, synthetic construct
VRDYVGMPYYSGSAYEY

SEQ ID NO: 751 - protein, 7 aa, synthetic construct
IRPGTIP

SEQ ID NO: 752 - protein, 16 aa, synthetic construct
AIRPGTIPYYSESVKG

SEQ ID NO: 753 - protein, 8 aa, synthetic construct
GLTFSSYG

SEQ ID NO: 754 - protein, 5 aa, synthetic construct
SYGMA

SEQ ID NO: 755 - protein, 818 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 756 - protein, 19 aa, synthetic construct
MMGVKVLFALICIAVAEAD

SEQ ID NO: 757 - protein, 208 aa, synthetic construct; identical to SEQ ID NO: 680

SEQ ID NO: 758 - protein, 43 aa, synthetic construct; identical to SEQ ID NO: 696

SEQ ID NO: 759 - protein, 119 aa, synthetic construct; identical to SEQ ID NO: 671

SEQ ID NO: 760 - protein, 20 aa, synthetic construct
GGGGSGGGGSGGGGSGGGGS

SEQ ID NO: 761 - protein, 106 aa, synthetic construct; identical to SEQ ID NO: 672

SEQ ID NO: 762 - protein, 244 aa, synthetic construct; identical to SEQ ID NO: 684

SEQ ID NO: 763 - protein, 823 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 764 - protein, 208 aa, synthetic construct; identical to SEQ ID NO: 680

SEQ ID NO: 765 - protein, 43 aa, synthetic construct; identical to SEQ ID NO: 696

SEQ ID NO: 766 - protein, 106 aa, synthetic construct; identical to SEQ ID NO: 672

SEQ ID NO: 767 - protein, 119 aa, synthetic construct; identical to SEQ ID NO: 671

SEQ ID NO: 768 - protein, 244 aa, synthetic construct; identical to SEQ ID NO: 684

SEQ ID NO: 769 - protein, 827 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLVQSGGGLVQPGGPLRLSCTASGFSFSNNYYMCWVRQAPGKGLEWVSCIYTNNIGSTWYASWAKGRFTISRDNSKNTLYLQMNSLRAEDTAVYYCARGIYSDSSDFIGNLWGQGTLVTVSSGGGGSGGGGSGGGGSGGGGSAIQMTQSPSSLSASVGDRVTITCQASQTIYNYLAWYQQKPGKVPELLIYKASTLASGVPSRFMGTGFGTDFTLTIDSLQPEDAATYYCQQYNTIINVDRTFGGGTKVEIKGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 770 - protein, 208 aa, synthetic construct; identical to SEQ ID NO: 680

SEQ ID NO: 771 - protein, 43 aa, synthetic construct; identical to SEQ ID NO: 696

SEQ ID NO: 772 - protein, 124 aa, synthetic construct; identical to SEQ ID NO: 703

SEQ ID NO: 773 - protein, 110 aa, synthetic construct; identical to SEQ ID NO: 704

SEQ ID NO: 774 - protein, 244 aa, synthetic construct; identical to SEQ ID NO: 684

SEQ ID NO: 775 - protein, 1058 aa, synthetic construct
YIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCMMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCQASQTIYNYLAWYQQKPGKVPELLIYKASTLASGVPSRFMGTGFGTDFTLTIDSLQPEDAATYYCQQYNTIINVDRTFGGGTKVEIKGGGGSGGGGSGGGGSGGGGSGGGGSQVQLVQSGGGLVQPGGPLRLSCTASGFSFSNNYYMCWVRQAPGKGLEWVSCIYTNNIGSTWYASWAKGRFTISRDNSKNTLYLQMNSLRAEDTAVYYCARGIYSDSSDFIGNLWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 776 - protein, 208 aa, synthetic construct; identical to SEQ ID NO: 680

SEQ ID NO: 777 - protein, 43 aa, synthetic construct; identical to SEQ ID NO: 696

SEQ ID NO: 778 - protein, 110 aa, synthetic construct; identical to SEQ ID NO: 704

SEQ ID NO: 779 - protein, 124 aa, synthetic construct; identical to SEQ ID NO: 703

SEQ ID NO: 780 - protein, 244 aa, synthetic construct; identical to SEQ ID NO: 684

SEQ ID NO: 781 - protein, 817 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSGGGGSGGGGSGGGGSQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGQTNYGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 782 - protein, 208 aa, synthetic construct; identical to SEQ ID NO: 680

SEQ ID NO: 783 - protein, 119 aa, synthetic construct; identical to SEQ ID NO: 671

SEQ ID NO: 784 - protein, 106 aa, synthetic construct; identical to SEQ ID NO: 672

SEQ ID NO: 785 - protein, 243 aa, synthetic construct
AGVTQSPTHLIKTRGQQVTLRCSPKSGHDTVSWYQQALGQGPQFIFQYYEEEERQRGNFPDRFSGHQFPNYSSELNVNALLLGDSALYLCASSLGQTNYGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADC

SEQ ID NO: 786 - protein, 826 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLVQSGGGLVQPGGPLRLSCTASGFSFSNNYYMCWVRQAPGKGLEWVSCIYTNNIGSTWYASWAKGRFTISRDNSKNTLYLQMNSLRAEDTAVYYCARGIYSDSSDFIGNLWGQGTLVTVSSGGGGSGGGGSGGGGSGGGGSAIQMTQSPSSLSASVGDRVTITCQASQTIYNYLAWYQQKPGKVPELLIYKASTLASGVPSRFMGTGFGTDFTLTIDSLQPEDAATYYCQQYNTIINVDRTFGGGTKVEIKGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGQTNYGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 787 - protein, 208 aa, synthetic construct; identical to SEQ ID NO: 680

SEQ ID NO: 788 - protein, 43 aa, synthetic construct; identical to SEQ ID NO: 696

SEQ ID NO: 789 - protein, 110 aa, synthetic construct; identical to SEQ ID NO: 704

SEQ ID NO: 790 - protein, 243 aa, synthetic construct; identical to SEQ ID NO: 785

SEQ ID NO: 791 - protein, 1098 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 792 - protein, 1098 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 793 - protein, 1106 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 794 - protein, 1103 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 795 - protein, 1084 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 796 - protein, 1099 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 797 - protein, 1092 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 798 - protein, 1107 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 799 - protein, 1094 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 800 - protein, 1083 aa, synthetic construct
MMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 801 - protein, 966 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 802 - protein, 966 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGLNDIFEAQKIEWHEGGGGSGGGGSLPETGG

SEQ ID NO: 803 - protein, 970 aa, synthetic construct
MMGVKVLFALICIAVAEAQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 804 - protein, 494 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSLEVLFQGPGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSHHHHHHHHHH

SEQ ID NO: 805 - protein, 534 aa, synthetic construct
MMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGSLEVLFQGPGGGGSGGGGSGGGGSDKTHTCPPCPAPELLGGPSVFLFPPKPKDTLEASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNAYTQKSLSLSPGKGGGGSGLNDIFEAQKIEWHE

SEQ ID NO: 806 - protein, 528 aa, synthetic construct
MMGVKVLFALICIAVAEALAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAGSGGLNDIFEAQKIEWHE

SEQ ID NO: 807 - protein, 534 aa, synthetic construct
MMGVKVLFALICIAVAEALAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGECGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCGSGGLNDIFEAQKIEWHE

SEQ ID NO: 808 - protein, 528 aa, synthetic construct
MMGVKVLFALICIAVAEALAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEGSGGLNDIFEAQKIEWHE

SEQ ID NO: 809 - protein, 534 aa, synthetic construct
MMGVKVLFALICIAVAEALAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGECGSGGLNDIFEAQKIEWHE

SEQ ID NO: 810 - protein, 273 aa, synthetic construct
MMGVKVLFALICIAVAEAEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDGGGGSHHHHHHHHHH

SEQ ID NO: 811 - protein, 263 aa, synthetic construct
MMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGLNDIFEAQKIEWHE

SEQ ID NO: 812 - protein, 565 aa, synthetic construct
MMGVKVLFALICIAVAEAEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDSGSGRIRRGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGSGGLNDIFEAQKIEWHEGSGHHHHHHHHHH

SEQ ID NO: 813 - protein, 529 aa, synthetic construct
MMGVKVLFALICIAVAEAEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDGGGGSGGGGSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGSGGLNDIFEAQKIEWHEGSGHHHHHHHHHH

SEQ ID NO: 814 - protein, 530 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGSGGLNDIFEAQKIEWHEGSGHHHHHHHHHH

SEQ ID NO: 815 - protein, 697 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 816 - protein, 702 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 817 - protein, 707 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 818 - protein, 961 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 819 - protein, 832 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 820 - protein, 831 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 821 - protein, 832 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 822 - protein, 837 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 823 - protein, 836 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 824 - protein, 837 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGLNDIFEAQKIEWHEGGGGSGGGGSLPETGG

SEQ ID NO: 825 - protein, 831 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGLNDIFEAQKIEWHEGGGGSGGGGSLPETGG

SEQ ID NO: 826 - protein, 1093 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 827 - protein, 1101 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 828 - protein, 1094 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 829 - protein, 1102 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 830 - protein, 1223 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 831 - protein, 1231 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 832 - protein, 1222 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 833 - protein, 1230 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 834 - protein, 1227 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 835 - protein, 1235 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 836 - protein, 1228 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGLNDIFEAQKIEWHEGGGGSGGGGSLPETGG

SEQ ID NO: 837 - protein, 1236 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGLNDIFEAQKIEWHEGGGGSGGGGSLPETGG

SEQ ID NO: 838 - protein, 1098 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 839 - protein, 1106 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 840 - protein, 1099 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 841 - protein, 1107 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 842 - protein, 1233 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 843 - protein, 1241 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 844 - protein, 1094 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 845 - protein, 1102 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 846 - protein, 1228 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 847 - protein, 1236 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 848 - protein, 1229 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGLNDIFEAQKIEWHEGGGGSGGGGSLPETGG

SEQ ID NO: 849 - protein, 1237 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGLNDIFEAQKIEWHEGGGGSGGGGSLPETGG

SEQ ID NO: 850 - protein, 1363 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGLNDIFEAQKIEWHEGGGGSGGGGSLPETGG

SEQ ID NO: 851 - protein, 1371 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGSGHHHHHHHHHHRIRRSGSGVKQTLNFDLLKLAGDVESNPGPMMGVKVLFALICIAVAEAEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSQVQLVESGGGLVQAGGSLRLSCAASGLTFSSYGMAWFRRALGREREFVGAIRPGTIPYYSESVKGRFTVSKDNAKNTVSLQMNSLKPEDTAVYYCAAVRDYVGMPYYSGSAYEYWGQGTQVTVSSGGGGSGLNDIFEAQKIEWHEGGGGSGGGGSLPETGG

SEQ ID NO: 852 - protein, 1227 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSGGGGSGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 853 - protein, 1235 aa, synthetic construct
MMGVKVLFALICIAVAEASQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAGGGGSGGGGSGGGGSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSGGGGSGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 854 - protein, 836 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGG

SEQ ID NO: 855 - protein, 965 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGSGHHHHHHHHHHRIRRSGSGVKQTLNFDLLKLAGDVESNPGPMMGVKVLFALICIAVAEAAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGGGGSEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSGGGGSGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGGGGSGLNDIFEAQKIEWHEGGGGSGGGGSLPETGG

SEQ ID NO: 856 - protein, 569 aa, synthetic construct
MMGVKVLFALICIAVAEADEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDGGGGSGGGGSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGGHHHHHHHHHH

SEQ ID NO: 857 - protein, 564 aa, synthetic construct
MMGVKVLFALICIAVAEADEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSGGGGSGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDGGGGSGGGGSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGGGGSGLNDIFEAQKIEWHEGGGGSGGGGSLPETGGHHHHHHHHHH

SEQ ID NO: 858 - protein, 822 aa, synthetic construct
MMGVKVLFALICIAVAEADEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSGGGGSGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDGGGGSGGGGSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGGGGSEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSGGGGSGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGGGGSGLNDIFEAQKIEWHEGGGGSGGGGSLPETGGHHHHHHHHHH

SEQ ID NO: 859 - protein, 698 aa, synthetic construct
MMGVKVLFALICIAVAEADEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSGGGGSGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDGGGGSGGGGSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGGGGSGGGGSEVQLVESGGGPVQAGGSLRLSCAASGRTYRGYSMAWFRQSPGKEREFVAAIVWSDGNTYYEDFVKGRFTISRDSAKNTLYLQMTNLKPEDTALYYCAAKIRPYIFKIAGQYDYWGQGTQVTVSSGGGGSGLNDIFEAQKIEWHEGGGGSGGGGSLPETGGHHHHHHHHHH

SEQ ID NO: 860 - protein, 538 aa, synthetic construct
MMGVKVLFALICIAVAEAEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDGGGGSGGGGSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGSGGLNDIFEAQKIEWHEGSGHHHHHHHHHH

SEQ ID NO: 861 - protein, 539 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDGGGGSEVRLQQSGPDLIKPGASVKMSCKASGYTFTGYVMHWVYKQRPGQGLEWIGFINPYNDDIQSNERFRGKATLTSDKSSTTAYMELSSLTSEDSAVYYCARGAGYNFDGAYRFEDFWGQGTTLTVSSGGGGSGGGGSGGGGSDVVMTQSPLSLPYSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVGSGGLNDIFEAQKIEWHEGSGHHHHHHHHHH

SEQ ID NO: 862 - protein, 783 aa, synthetic construct
MMGVKVLFALICIAVAEAAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRADGGGGSGGGGSGGGGSGGGGSGGGGSGGGGSGGGGSLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKSVCLFTDFDSQTQVSQSKDSDVYITDKCVLDMRSMDFKSNSAVAWSQKSDFACANAFQNSIIPEDTFFPSPESSGSGLPETGGGSGHHHHHHHHHH

SEQ ID NO: 863 - protein, 130 aa, synthetic construct
EDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRAD

SEQ ID NO: 864 - protein, 35 aa, synthetic construct
GGGGSGGGGSGGGGSGGGGSGGGGSGGGGSGGGGS

SEQ ID NO: 865 - protein, 94 aa, synthetic construct
YIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 866 - protein, 10 aa, synthetic construct
HHHHHHHHHH

SEQ ID NO: 867 - protein, 769 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKSVCLFTDFDSQTQVSQSKDSDVYITDKCVLDMRSMDFKSNSAVAWSQKSDFACANAFQNSIIPEDTFFPSPESSGGGGSGGGGSGGGGSGGGGSAIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRADGSGLPETGGGSGHHHHHHHHHH

SEQ ID NO: 868 - protein, 94 aa, synthetic construct; identical to SEQ ID NO: 865

SEQ ID NO: 869 - protein, 20 aa, synthetic construct
GGGGSGGGGSGGGGSGGGGS

SEQ ID NO: 870 - protein, 24 aa, synthetic construct
GGGGSGGGGSGGGGSGGGGSGGGS

SEQ ID NO: 871 - protein, 130 aa, synthetic construct; identical to SEQ ID NO: 863

SEQ ID NO: 872 - protein, 37 aa, synthetic construct
  VARIANT 8..32: note = A, G, S, or T
GGSSGSGXXXXXXXXXXXXXXXXXXXXXXXXXGSGSG

SEQ ID NO: 873 - protein, 34 aa, synthetic construct
SGGGSGGGSEGGGSEGGGSEGGGSEGGGSGGGSG

SEQ ID NO: 874 - protein, 7 aa, synthetic construct
NIATNDY

SEQ ID NO: 875 - protein, 5 aa, synthetic construct
GYKTK

SEQ ID NO: 876 - protein, 13 aa, synthetic construct
LVGDMDQAGTALI

SEQ ID NO: 877 - protein, 5 aa, synthetic construct
SGHDT

SEQ ID NO: 878 - protein, 6 aa, synthetic construct
YYEEEE

SEQ ID NO: 879 - protein, 13 aa, synthetic construct
ASSLGEGRVDGYT

SEQ ID NO: 880 - protein, 5 aa, synthetic construct
SSVSY

SEQ ID NO: 881 - skipped

SEQ ID NO: 882 - protein, 9 aa, synthetic construct
QQWSSNPLT

SEQ ID NO: 883 - protein, 110 aa, synthetic construct
LAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSV

SEQ ID NO: 884 - protein, 127 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGS

SEQ ID NO: 885 - protein, 9 aa, synthetic construct
YLEPGPVTA

SEQ ID NO: 886 - protein, 8 aa, synthetic construct
DTAGHEEY

SEQ ID NO: 887 - protein, 10 aa, synthetic construct
TAGHEEYSAM

SEQ ID NO: 888 - protein, 9 aa, synthetic construct
DILDTAGHE

SEQ ID NO: 889 - protein, 8 aa, synthetic construct
DILDTAGH

SEQ ID NO: 890 - protein, 9 aa, synthetic construct
ILDTAGHEE

SEQ ID NO: 891 - protein, 8 aa, synthetic construct
ILDTAGHE

SEQ ID NO: 892 - protein, 11 aa, synthetic construct
DILDTAGHEEY

SEQ ID NO: 893 - protein, 9 aa, synthetic construct
DTAGHEEYS

SEQ ID NO: 894 - protein, 10 aa, synthetic construct
LLDILDTAGH

SEQ ID NO: 895 - protein, 9 aa, synthetic construct
DILDTAGRE

SEQ ID NO: 896 - protein, 8 aa, synthetic construct
DILDTAGR

SEQ ID NO: 897 - protein, 9 aa, synthetic construct
ILDTAGREE

SEQ ID NO: 898 - protein, 8 aa, synthetic construct
ILDTAGRE

SEQ ID NO: 899 - protein, 11 aa, synthetic construct
CLLDILDTAGR

SEQ ID NO: 900 - protein, 10 aa, synthetic construct
TAGREEYSAM

SEQ ID NO: 901 - protein, 9 aa, synthetic construct
REEYSAMRD

SEQ ID NO: 902 - protein, 11 aa, synthetic construct
DTAGKEEYSAM

SEQ ID NO: 903 - protein, 11 aa, synthetic construct
CLLDILDTAGK

SEQ ID NO: 904 - protein, 8 aa, synthetic construct
DTAGKEEY

SEQ ID NO: 905 - protein, 10 aa, synthetic construct
LLDILDTAGK

SEQ ID NO: 906 - protein, 8 aa, synthetic construct
ILDTAGKE

SEQ ID NO: 907 - protein, 9 aa, synthetic construct
ILDTAGKEE

SEQ ID NO: 908 - protein, 8 aa, synthetic construct
DTAGLEEY

SEQ ID NO: 909 - protein, 8 aa, synthetic construct
ILDTAGLE

SEQ ID NO: 910 - protein, 8 aa, synthetic construct
DILDTAGL

SEQ ID NO: 911 - protein, 9 aa, synthetic construct
ILDTAGLEE

SEQ ID NO: 912 - protein, 12 aa, synthetic construct
GLEEYSAMRDQY

SEQ ID NO: 913 - protein, 11 aa, synthetic construct
LLDILDTAGLE

SEQ ID NO: 914 - protein, 9 aa, synthetic construct
LDILDTAGL

SEQ ID NO: 915 - protein, 9 aa, synthetic construct
DILDTAGLE

SEQ ID NO: 916 - protein, 11 aa, synthetic construct
DILDTAGLEEY

SEQ ID NO: 917 - protein, 8 aa, synthetic construct
AGVGKSAL

SEQ ID NO: 918 - protein, 10 aa, synthetic construct
GAAGVGKSAL

SEQ ID NO: 919 - protein, 9 aa, synthetic construct
AAGVGKSAL

SEQ ID NO: 920 - protein, 8 aa, synthetic construct
CGVGKSAL

SEQ ID NO: 921 - protein, 9 aa, synthetic construct
ACGVGKSAL

SEQ ID NO: 922 - protein, 8 aa, synthetic construct
DGVGKSAL

SEQ ID NO: 923 - protein, 9 aa, synthetic construct
ADGVGKSAL

SEQ ID NO: 924 - protein, 10 aa, synthetic construct
DGVGKSALTI

SEQ ID NO: 925 - protein, 9 aa, synthetic construct
GARGVGKSA

SEQ ID NO: 926 - protein, 10 aa, synthetic construct
KLVVVGARGV

SEQ ID NO: 927 - protein, 8 aa, synthetic construct
VVVGARGV

SEQ ID NO: 928 - protein, 8 aa, synthetic construct
SGVGKSAL

SEQ ID NO: 929 - protein, 10 aa, synthetic construct
VVVGASGVGK

SEQ ID NO: 930 - protein, 10 aa, synthetic construct
GASGVGKSAL

SEQ ID NO: 931 - protein, 8 aa, synthetic construct
VGVGKSAL

SEQ ID NO: 932 - protein, 10 aa, synthetic construct
VVVGAGCVGK

SEQ ID NO: 933 - protein, 9 aa, synthetic construct
KLVVVGAGC

SEQ ID NO: 934 - protein, 8 aa, synthetic construct
GDVGKSAL

SEQ ID NO: 935 - protein, 9 aa, synthetic construct
DVGKSALTI

SEQ ID NO: 936 - protein, 10 aa, synthetic construct
VVVGAGDVGK

SEQ ID NO: 937 - protein, 10 aa, synthetic construct
TAGKEEYSAM

SEQ ID NO: 938 - protein, 11 aa, synthetic construct
DTAGHEEYSAM

SEQ ID NO: 939 - protein, 9 aa, synthetic construct
TAGHEEYSA

SEQ ID NO: 940 - protein, 11 aa, synthetic construct
DTAGREEYSAM

SEQ ID NO: 941 - protein, 9 aa, synthetic construct
TAGKEEYSA

SEQ ID NO: 942 - protein, 8 aa, synthetic construct
AAGVGKSA

SEQ ID NO: 943 - protein, 9 aa, synthetic construct
AGCVGKSAL

SEQ ID NO: 944 - protein, 9 aa, synthetic construct
AGDVGKSAL

SEQ ID NO: 945 - protein, 10 aa, synthetic construct
AGKEEYSAMR

SEQ ID NO: 946 - protein, 10 aa, synthetic construct
AGVGKSALTI

SEQ ID NO: 947 - protein, 9 aa, synthetic construct
ARGVGKSAL

SEQ ID NO: 948 - protein, 8 aa, synthetic construct
ASGVGKSA

SEQ ID NO: 949 - protein, 9 aa, synthetic construct
ASGVGKSAL

SEQ ID NO: 950 - protein, 8 aa, synthetic construct
AVGVGKSA

SEQ ID NO: 951 - protein, 9 aa, synthetic construct
CVGKSALTI

SEQ ID NO: 952 - protein, 8 aa, synthetic construct
DILDTAGK

SEQ ID NO: 953 - protein, 11 aa, synthetic construct
DILDTAGREEY

SEQ ID NO: 954 - protein, 12 aa, synthetic construct
DTAGHEEYSAMR

SEQ ID NO: 955 - protein, 9 aa, synthetic construct
DTAGKEEYS

SEQ ID NO: 956 - protein, 12 aa, synthetic construct
DTAGKEEYSAMR

SEQ ID NO: 957 - protein, 9 aa, synthetic construct
DTAGLEEYS

SEQ ID NO: 958 - protein, 10 aa, synthetic construct
DTAGLEEYSA

SEQ ID NO: 959 - protein, 12 aa, synthetic construct
DTAGLEEYSAMR

SEQ ID NO: 960 - protein, 9 aa, synthetic construct
DTAGREEYS

SEQ ID NO: 961 - protein, 12 aa, synthetic construct
DTAGREEYSAMR

SEQ ID NO: 962 - protein, 9 aa, synthetic construct
GAAGVGKSA

SEQ ID NO: 963 - protein, 9 aa, synthetic construct
GACGVGKSA

SEQ ID NO: 964 - protein, 10 aa, synthetic construct
GACGVGKSAL

SEQ ID NO: 965 - protein, 8 aa, synthetic construct
GADGVGKS

SEQ ID NO: 966 - protein, 9 aa, synthetic construct
GAGDVGKSA

SEQ ID NO: 967 - protein, 10 aa, synthetic construct
GAGDVGKSAL

SEQ ID NO: 968 - protein, 9 aa, synthetic construct
GASGVGKSA

SEQ ID NO: 969 - protein, 8 aa, synthetic construct
GCVGKSAL

SEQ ID NO: 970 - protein, 10 aa, synthetic construct
GCVGKSALTI

SEQ ID NO: 971 - protein, 8 aa, synthetic construct
GHEEYSAM

SEQ ID NO: 972 - protein, 8 aa, synthetic construct
GKEEYSAM

SEQ ID NO: 973 - protein, 9 aa, synthetic construct
GLEEYSAMR

SEQ ID NO: 974 - protein, 8 aa, synthetic construct
GREEYSAM

SEQ ID NO: 975 - protein, 9 aa, synthetic construct
GREEYSAMR

SEQ ID NO: 976 - protein, 9 aa, synthetic construct
HEEYSAMRD

SEQ ID NO: 977 - protein, 9 aa, synthetic construct
KEEYSAMRD

SEQ ID NO: 978 - protein, 9 aa, synthetic construct
KLVVVGASG

SEQ ID NO: 979 - protein, 9 aa, synthetic construct
LDILDTAGR

SEQ ID NO: 980 - protein, 9 aa, synthetic construct
LEEYSAMRD

SEQ ID NO: 981 - protein, 9 aa, synthetic construct
LVVVGARGV

SEQ ID NO: 982 - protein, 9 aa, synthetic construct
LVVVGASGV

SEQ ID NO: 983 - protein, 11 aa, synthetic construct
REEYSAMRDQY

SEQ ID NO: 984 - protein, 8 aa, synthetic construct
RGVGKSAL

SEQ ID NO: 985 - protein, 9 aa, synthetic construct
TAGLEEYSA

SEQ ID NO: 986 - protein, 11 aa, synthetic construct
TEYKLVVVGAA

SEQ ID NO: 987 - protein, 10 aa, synthetic construct
VGAAGVGKSA

SEQ ID NO: 988 - protein, 8 aa, synthetic construct
VGADGVGK

SEQ ID NO: 989 - protein, 10 aa, synthetic construct
VGASGVGKSA

SEQ ID NO: 990 - protein, 10 aa, synthetic construct
VGVGKSALTI

SEQ ID NO: 991 - protein, 8 aa, synthetic construct
VVVGAAGV

SEQ ID NO: 992 - protein, 8 aa, synthetic construct
VVVGAVGV

SEQ ID NO: 993 - protein, 9 aa, synthetic construct
YKLVVVGAC

SEQ ID NO: 994 - protein, 9 aa, synthetic construct
YKLVVVGAD

SEQ ID NO: 995 - protein, 9 aa, synthetic construct
YKLVVVGAR

SEQ ID NO: 996 - protein, 9 aa, synthetic construct
DILDTAGKE

SEQ ID NO: 997 - protein, 10 aa, synthetic construct
GADGVGKSAL

SEQ ID NO: 998 - protein, 10 aa, synthetic construct
GAVGVGKSAL

SEQ ID NO: 999 - protein, 9 aa, synthetic construct
GADGVGKSA

SEQ ID NO: 1000 - protein, 9 aa, synthetic construct
GAVGVGKSA

SEQ ID NO: 1001 - protein, 10 aa, synthetic construct
ILDTAGHEEY

SEQ ID NO: 1002 - protein, 9 aa, synthetic construct
AGHEEYSAM

SEQ ID NO: 1003 - protein, 9 aa, synthetic construct
LDTAGHEEY

SEQ ID NO: 1004 - protein, 10 aa, synthetic construct
DTAGHEEYSA

SEQ ID NO: 1005 - protein, 23 aa, synthetic construct
TCLLDILDTAGREEYSAMRDQYM

SEQ ID NO: 1006 - protein, 10 aa, synthetic construct
ILDTAGREEY

SEQ ID NO: 1007 - protein, 8 aa, synthetic construct
DTAGREEY

SEQ ID NO: 1008 - protein, 9 aa, synthetic construct
AGREEYSAM

SEQ ID NO: 1009 - protein, 9 aa, synthetic construct
TAGREEYSA

SEQ ID NO: 1010 - protein, 10 aa, synthetic construct
LLDILDTAGR

SEQ ID NO: 1011 - protein, 10 aa, synthetic construct
AGREEYSAMR

SEQ ID NO: 1012 - protein, 9 aa, synthetic construct
LDTAGREEY

SEQ ID NO: 1013 - protein, 23 aa, synthetic construct
TCLLDILDTAGKEEYSAMRDQYM

SEQ ID NO: 1014 - protein, 10 aa, synthetic construct
ILDTAGKEEY

SEQ ID NO: 1015 - protein, 9 aa, synthetic construct
AGKEEYSAM

SEQ ID NO: 1016 - protein, 9 aa, synthetic construct
LDTAGKEEY

SEQ ID NO: 1017 - protein, 23 aa, synthetic construct
TCLLDILDTAGLEEYSAMRDQYM

SEQ ID NO: 1018 - protein, 10 aa, synthetic construct
ILDTAGLEEY

SEQ ID NO: 1019 - protein, 10 aa, synthetic construct
LLDILDTAGL

SEQ ID NO: 1020 - protein, 11 aa, synthetic construct
DTAGLEEYSAM

SEQ ID NO: 1021 - protein, 11 aa, synthetic construct
CLLDILDTAGL

SEQ ID NO: 1022 - protein, 9 aa, synthetic construct
LDTAGLEEY

SEQ ID NO: 1023 - protein, 9 aa, synthetic construct
AGLEEYSAM

SEQ ID NO: 1024 - protein, 10 aa, synthetic construct
AGLEEYSAMR

SEQ ID NO: 1025 - protein, 8 aa, synthetic construct
GLEEYSAM

SEQ ID NO: 1026 - protein, 6 aa, synthetic construct
GGGGGG

SEQ ID NO: 1027 - protein, 8 aa, synthetic construct
GGGGGGGG

SEQ ID NO: 1028 - protein, 5 aa, synthetic construct
EAAAK

SEQ ID NO: 1029 - protein, 4 aa, synthetic construct
GSGS

SEQ ID NO: 1030 - protein, 4 aa, synthetic construct
SGGG

SEQ ID NO: 1031 - protein, 6 aa, synthetic construct
HHHHHH

SEQ ID NO: 1032 - protein, 94 aa, synthetic construct
YIQNPDPAVYQLRDSKSSDKFVCLFTDFDSQIQVSQSKDSDVYITDKCVLDMRSMDFKSNSAVAWSQKSDFTCANAFQNSIIPEDTFFPSPESS

SEQ ID NO: 1033 - protein, 149 aa, synthetic construct
EDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGSGGLNDIFEAQKIEWHE

SEQ ID NO: 1034 - protein, 804 aa, synthetic construct
LAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAQIVLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSGGGGSGGGGSQVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSGGGGSAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

SEQ ID NO: 1035 - protein, 5 aa, synthetic construct
LPETG

SEQ ID NO: 1036 - protein, 6 aa, synthetic construct
SLPETG

SEQ ID NO: 1037 - protein, 35 aa, synthetic construct; identical to SEQ ID NO: 864
  VARIANT 1..35: note = This sequence may encompass 1, 4, 5, or 7 GGGGS repeating units

SEQ ID NO: 1038 - protein, 832 aa, synthetic construct
MMGVKVLFALICIAVAEADLAKTTQPISVDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDYIQNPDPAVYQLRDSKSSDKFVCLFTDFDSQIQVSQSKDSDVYITDKCVLDMRSMDFKSNSAVAWSQKSDFTCANAFQNSIIPEDTFFPSPESSCGSGHHHHHHHHHHRIRRSGSGVKQTLNFDLLKLAGDVESNPGPMMGVKVLFALICIAVAEAAIQMTQSPSSLSASVGDRVTITCQASQTIYNYLAWYQQKPGKVPELLIYKASTLASGVPSRFMGTGFGTDFTLTIDSLQPEDAATYYCQQYNTIINVDRTFGGGTKVEIKGGGGSGGGGSGGGGSGGGGSGGGGSQVQLVQSGGGLVQPGGPLRLSCTASGFSFSNNYYMCWVRQAPGKGLEWVSCIYTNNIGSTWYASWAKGRFTISRDNSKNTLYLQMNSLRAEDTAVYYCARGIYSDSSDFIGNLWGQGTLVTVSSGGGGSAGVTQSPTHLIKTRGQQVTLRCSPKSGHDTVSWYQQALGQGPQFIFGYYEEEERQRGNFPDRFSGHQFPNYSSELNVNALLLGDSALYLCASSLGEGRVDGYTFGSGTRLTVVEDLKNVFPPEVAVFEPSKAEISRTQKATLVCLATGFYPPHVELSWWVNGKEVHDGVCTDPQPLKEQPALQDSRYALSSRLRVSATFWQDPRNHFRCQVQFYGLSENDEWTQDRAKPVTQIVSAEAWGRADCGGGGSGLNDIFEAQKIEWHEGGGGSGGGGSLPETGG

(19) United States
(12) Patent Application Publication
(10) Pub. No.: US 2026/0347430 A1
(43) Pub. Date: Nov. 5, 2026

(54) MULTISPECIFIC T CELL ENGAGERS COMPOSITIONS AND METHODS OF USE THEREOF

(71) Applicant: BioNTech SE, Mainz (DE)

(72) Inventors: John Srouji, Somerville, MA (US); Charles Lefco Dulberger, Cambridge, MA (US); Benjamin P. Roscoe, Waltham, MA (US); Richard Gaynor, Carmel, IN (US); Ugur Sahin, Mainz (DE); Karsten Beckmann, Vaterstetten (DE); Aline Häcker, Mainz (DE)

(21) Appl. No.: 19/649,966

(22) Filed: Apr. 16, 2026

Related U.S. Application Data
(63) Continuation of application No. PCT/US2024/052124, filed on Oct. 18, 2024.
(60) Provisional application No. 63/592,013, filed on Oct. 20, 2023.
(60) Provisional application No. 63/698,816, filed on Sep. 25, 2024.

(51) Int. Cl. C07K 14/705; C07K 16/28

(52) U.S. Cl. CPC C07K 14/7051; C07K 14/70539; C07K 16/2809; C07K 2317/31; C07K 2317/622; C07K 2319/00

(57) ABSTRACT

Compositions and methods for making and using therapeutic agents comprising multispecific molecule, used for immunotherapy of cancer or infection.

### CROSS REFERENCE

This application is a continuation of International Application No. PCT/US2024/052124, filed on Oct. 24, 2024, which claims the benefit of U.S. Provisional Application No. 63/698,816, filed on Sep. 25, 2024, and U.S. Provisional Application No. 63/592,013, filed on Oct. 20, 2023, which are incorporated herein by reference in their entirety.

### SEQUENCE LISTING

The instant application contains a Sequence Listing which has been submitted electronically in XML format and is hereby incorporated by reference in its entirety. Said XML copy, created on Apr. 13, 2026, is named 50401-777_601_SL.xml and is 1,661,190 bytes in size.

### BACKGROUND

T cell receptors (TCRs) are important element for adaptive immune system for recognizing and eliminating “non-self” intracellular antigens. It is estimated that more than 70% of tumor-specific targets are processed intracellularly and presented as peptides on the cell surface in the context of major histocompatibility complexes (MHC), known as human leukocyte antigens (HLA) when referring to human MHC. These peptide HLA complexes (pHLAs) are inaccessible to antibody targeting including CAR T cells, antibody-drug conjugates, and bispecific T cell engagers but they are recognized by T cells through their TCRs. In addition, these pHLAs can also be recognized by allogeneic T cells that contain the same HLA alleles through TCR-peptide/HLA interactions. Thus, TCR-based therapies have become promising tools to eliminate cells expressing mutated tumor-specific or -associated peptides derived from intracellular proteins and presented in the context of MHCs on the surface of tumor cells. However, the weak affinity and low stability of naturally selected TCRs impose certain challenges on their use for therapeutic applications. Consequently, the most widely used TCR-based therapies involve the adoptive transfer of either expanded antigen-specific T cells or T cells genetically modified to express an artificial antigen-specific TCR (specific peptide affinity-enhanced receptor [SPEAR]). These therapies are complicated by the need to prepare therapeutic T cells on a patient-by-patient basis.

### SUMMARY

In one aspect, provided herein is a composition comprising a recombinant nucleic acid for expression in a mammalian cell, the recombinant nucleic acid comprising a sequence encoding a multispecific molecule, wherein the multispecific molecule is an engineered T cell receptor (TCR) construct, comprising: (i) an MHC-peptide complex engager comprising: (a) a T cell receptor (TCR) alpha variable (TRAV) domain, and (b) a TCR beta variable (TRBV) domain; and (ii) a T cell engager comprising one or more binding domains that bind to an extracellular domain of a receptor expressed a T cell, wherein the T cell engager comprises an anti-CD3 binding domain comprising a heavy chain variable region (VH) that comprises a heavy chain complementarity determining region 3 (HC CDR3) sequence of ARGIYSDSSDFIGNL (SEQ ID NO: 713) or GIYSDSSDFIGNL (SEQ ID NO: 707).

In another aspect, provided herein is a composition comprising a recombinant nucleic acid comprising a sequence encoding a multispecific molecule, wherein the multispecific molecule comprises: (i) an MHC-peptide complex engager comprising: (a) a TCR alpha variable (TRAV) domain, and (b) a TCR beta variable (TRBV) domain; wherein the peptide in the MHC-peptide complex comprises a RAS peptide sequence; and (ii) a T cell engager comprising one or more binding domains that bind to an extracellular domain of a receptor expressed by a T cell, wherein the T cell engager comprises an anti-CD3 binding domain comprising a heavy chain variable region (VH) that comprises a heavy chain complementarity determining region 3 (HC CDR3) sequence of ARGIYSDSSDFIGNL (SEQ ID NO: 713) or GIYSDSSDFIGNL (SEQ ID NO: 707). In some embodiments, the multispecific molecule comprises a post-translational modification. In some embodiments, the multispecific molecule comprises a mammalian glycosylation signature. In some embodiments, the multispecific molecule is soluble. In some embodiments, the multispecific molecule lacks a trans-membrane domain. In some embodiments, the multispecific molecule consists of two polypeptides, a first polypeptide and a second polypeptide. In some embodiments, the first polypeptide and the second polypeptide comprise one or more disulfide bonds between the two polypeptides. In some embodiments, the multispecific molecule comprises a first polypeptide and a second polypeptide, wherein the first polypeptide comprises the TRAV domain and the second polypeptide comprises the TRBV domain. In some embodiments, the peptide of the MHC-peptide complex is a peptide from a cancer antigen. In some embodiments, the peptide of the MHC-peptide complex is a mutant peptide, and wherein the MHC of the MHC-peptide complex binds the mutant peptide with a higher affinity compared to a corresponding wild-type peptide. In some embodiments, the peptide of the MHC-peptide complex does not comprise an amino acid sequence YLEPGPVTA. In some embodiments, the MHC of the MHC-peptide complex comprises a class I MHC. In some embodiments, the MHC of the MHC-peptide complex is a class I MHC polypeptide. In some embodiments, the MHC of the MHC-peptide complex is encoded by an HLA that is represented in less than 1% of a human population. In some embodiments, the MHC of the MHC-peptide complex has a peptide binding affinity greater than at least 50 nM. In some embodiments, the one or more binding domains of the T cell engager bind to an extracellular domain of an endogenous receptor expressed by a T cell. In some embodiments, the one or more binding domains of the T cell engager bind to an extracellular domain of a receptor selected from a group consisting of CD3, CD2, CD7, CD5, CD4, CD28, ICAM-1 and CD8. In some embodiments, the one or more binding domains of the T cell engager binds to CD3 delta, CD3 gamma or CD3 epsilon. In some embodiments, the one or more binding domains of the T cell engager comprises an antibody domain or antigen binding fragment thereof. In some embodiments, the binding domain of the T cell engager comprises an scFv or an sdAb. In some embodiments, the one or more binding domains of the T cell engager comprises a VHH.

In some embodiments, the one or more binding domains of the T cell engager comprises a first binding domain that binds to an extracellular domain of a first receptor expressed by a T cell and a second binding domain that binds to an extracellular domain of a second receptor expressed by a T cell. In some embodiments, the first binding domain of the T cell engager that binds to an extracellular domain of a first receptor expressed by a T cell comprises a first VHH, and the second binding domain of the T cell engager that binds to an extracellular domain of a second receptor expressed by a T cell comprises a second VHH. In one embodiment, the C-terminus of the first binding domain of the T cell engager is connected to the N-terminus of the second binding domain of the T cell engager; or the C-terminus of the first binding domain of the T cell engager is connected to the C-terminus of the second binding domain of the T cell engager. In some embodiments, the N-terminus of the first binding domain of the T cell engager is connected to the N-terminus of the second binding domain of the T cell engager; or the N-terminus of the first binding domain of the T cell engager is connected to the C-terminus of the second binding domain of the T cell engager. In some embodiments, the first binding domain of the T cell engager that binds to an extracellular domain of a first receptor expressed by a T cell comprises a scFv, and the second binding domain of the T cell engager that binds to an extracellular domain of a second receptor expressed by a T cell comprises a VHH. In some embodiments, the binding domain that binds to an extracellular domain of the first receptor expressed by a T cell and the second binding domain that binds to an extracellular domain of the second receptor expressed by a T cell are connected by a peptide linker. In some embodiments, the first binding domain binds to an extracellular domain of a first receptor expressed by a T cell selected from a group consisting of CD3, CD2, CD7, CD5, CD4, CD28, ICOS and CD8; and the second binding domain binds to an extracellular domain of a second receptor expressed by a T cell selected from a group consisting of CD3, CD2, CD7, CD5, CD4, CD28, ICOS and CD8. In some embodiments, the first receptor expressed by a T cell and the second receptor expressed by a T cell are different. In some embodiments, the first receptor expressed by a T cell is CD3 and the second receptor expressed by a T cell is CD2. In some embodiments, the first receptor expressed by a T cell is CD2 and the second receptor expressed by a T cell is CD3. In some embodiments, the first binding domain and the second binding domain of the T cell engager are configured to bind to the same receptor expressed by a T cell. In some embodiments, the VH of the anti-CD3 binding domain comprises a HC CDR2 sequence of IYTNNIGST (SEQ ID NO: 712) or CIYTNNIGSTWYASWAKG (SEQ ID NO: 706). In some embodiments, the VH of the anti-CD3 binding domain comprises a HC CDR1 sequence of GFSFSNNYY (SEQ ID NO: 711) or NNYYMC (SEQ ID NO: 705). In some embodiments, the anti-CD3 binding domain comprises a light chain variable region (VL) that comprises a light chain complementarity determining region 3 (LC CDR3) sequence of QQYNTIINVDRT (SEQ ID NO: 713). In some embodiments, the VL of the anti-CD3 binding domain comprises a LC CDR2 sequence of KAS (SEQ ID NO: 715) or KASTLAS (SEQ ID NO: 709). In some embodiments, the VL of the anti-CD3 binding domain comprises a LC CDR1 sequence of QTIYNY (SEQ ID NO: 714) or QASQTIYNYLA (SEQ ID NO: 708). In some embodiments, the anti-CD3 binding domain comprises: a HC CDR1 sequence of GFSFSNNYY (SEQ ID NO: 711), a HC CDR2 sequence of IYTNNIGST (SEQ ID NO: 712), a HC CDR3 sequence of ARGIYSDSSDFIGNL (SEQ ID NO: 713), a LC CDR1 sequence of QTIYNY (SEQ ID NO: 714), a LC CDR2 sequence of KAS (SEQ ID NO: 715), and a LC CDR3 sequence of QQYNTIINVDRT (SEQ ID NO: 716), wherein the CDR sequences are determined based on IMGT CDR numbering scheme. In some embodiments, the anti-CD3 binding domain comprises: a HC CDR1 sequence of NNYYMC (SEQ ID NO: 705), a HC CDR2 sequence of CIYTNNIGSTWYASWAKG (SEQ ID NO: 706), a HC CDR3 sequence of GIYSDSSDFIGNL (SEQ ID NO: 707), a LC CDR1 sequence of QASQTIYNYLA (SEQ ID NO: 708), a LC CDR2 sequence of KASTLAS (SEQ ID NO: 709), and a LC CDR3 sequence of QQYNTIINVDRT (SEQ ID NO: 710), wherein the CDR sequences are determined based on Kabat CDR numbering scheme.

In some embodiments, the VH of the anti-CD3 binding domain comprises a sequence with at least 80% sequence identity to the sequence QVQLVQSGGGLVQPGGPLRLSCTASGFSFSNNYYMCWVRQAPGKGLEWVSCIYTNNIGST WYASWAKGRFTISRDNSKNTLYLQMNSLRAEDTAVYYCARGIYSDSSDFIGNLWGQGTLV TVSS (SEQ ID NO: 703).

In some embodiments, the VL of the anti-CD3 binding domain comprises a sequence with at least 80% sequence identity to the sequence AIQMTQSPSSLSASVGDRVTITCQASQTIYNYLAWYQQKPGKVPELLIYKASTLASGVPSRF MGTGFGTDFTLTIDSLQPEDAATYYCQQYNTIINVDRTFGGGTKVEIK (SEQ ID NO: 704).

In some embodiments, the anti-CD3 binding domain comprises a sequence with at least 80% sequence identity to QVQLVQSGGGLVQPGGPLRLSCTASGFSFSNNYYMCWVRQAPGKGLEWVSCIYTNNIGST WYASWAKGRFTISRDNSKNTLYLQMNSLRAEDTAVYYCARGIYSDSSDFIGNLWGQGTLV TVSSGGGGSGGGGSGGGGSGGGGSAIQMTQSPSSLSASVGDRVTITCQASQTIYNYLAWYQ QKPGKVPELLIYKASTLASGVPSRFMGTGFGTDFTLTIDSLQPEDAATYYCQQYNTIINVDRT FGGGTKVEIK (SEQ ID NO: 701).

In some embodiments, the anti-CD3 binding domain comprises a sequence with at least 80% sequence identity to AIQMTQSPSSLSASVGDRVTITCQASQTIYNYLAWYQQKPGKVPELLIYKASTLASGVPSRF MGTGFGTDFTLTIDSLQPEDAATYYCQQYNTIINVDRTFGGGTKVEIKGGGGSGGGGSGGG GSGGGGSGGGGSQVQLVQSGGGLVQPGGPLRLSCTASGFSFSNNYYMCWVRQAPGKGLE WVSCIYTNNIGSTWYASWAKGRFTISRDNSKNTLYLQMNSLRAEDTAVYYCARGIYSDSSD FIGNLWGQGTLVTVSS (SEQ ID NO: 702).

In some embodiments, the T cell engager comprises an additional anti-CD3 binding domain. In some embodiments, the T cell engager further comprises an anti-TRBC1 binding domain. In some embodiments, the T cell engager further comprises an anti-CD2 binding domain.

In some embodiments, the T cell engager comprises an additional anti-CD3 binding domain comprising a heavy chain variable region (VH) that comprises a heavy chain complementarity determining region 3 (HC CDR3) sequence of ARYYDDHYCLDY (SEQ ID NO: 720), ARYYDDHYSLDY (SEQ ID NO: 721), YYDDHYCLDY (SEQ ID NO: 675) or YYDDHYSLDY (SEQ ID NO: 717). In some embodiments, the VH of the additional anti-CD3 binding domain comprises a HC CDR2 sequence of INPSRGYT (SEQ ID NO: 719) or YINPSRGYTNYNQKFKD (SEQ ID NO: 674).

In some embodiments, the VH of the additional anti-CD3 binding domain comprises a HC CDR1 sequence of GYTFTRYT (SEQ ID NO: 718) or RYTMH (SEQ ID NO: 673). In some embodiments, the additional anti-CD3 binding domain comprises a light chain variable region (VL) that comprises a light chain complementarity determining region 3 (LC CDR3) sequence of QQWSSNPLT (SEQ ID NO: 678). In some embodiments, the VL of the additional anti-CD3 binding domain comp In some embodiments, the VL of the additional anti-CD3 binding domain comprises a LC CDR1 sequence of SSVSY (SEQ ID NO: 722) or RASSSVSYMN (SEQ ID NO: 676). In some embodiments, the additional anti-CD3 binding domain comprises: a HC CDR1 sequence of GYTFTRYT (IMGT) (SEQ ID NO: 718), a HC CDR2 sequence of INPSRGYT (IMGT) (SEQ ID NO: 719), a HC CDR3 sequence of ARYYDDHYCLDY(IMGT) (SEQ ID NO: 720) or ARYYDDHYSLDY (SEQ ID NO: 721), a LC CDR1 sequence of SSVSY (SEQ ID NO: 722), a LC CDR2 sequence of DTS (SEQ ID NO: 723), and a LC CDR3 sequence of QQWSSNPLT (SEQ ID NO: 724).

In one aspect, the additional anti-CD3 binding domain comprises: a HC CDR1 sequence of RYTMH (SEQ ID NO: 673), a HC CDR2 sequence of YINPSRGYTNYNQKFKD (SEQ ID NO: 674), a HC CDR3 sequence of YYDDHYCLDY (SEQ ID NO: 675) or YYDDHYSLDY (SEQ ID NO: 717), a LC CDR1 sequence of RASSSVSYMN (SEQ ID NO: 676), a LC CDR2 sequence of DTSKVAS (SEQ ID NO: 677), and a LC CDR3 sequence of QQWSSNPLT (SEQ ID NO: 678). In some embodiments the VH of the additional anti-CD3 binding domain comprises a sequence with at least 80% sequence identity to the sequence QVQLQQSGAELARPGASVKMSCKTSGYTFTRYTMHWVKQRPGQGLEWIGYINPSRGYTNY NQKFKDKATLTTDKSSSTAYMQLSSLTSEDSAVYYCARYYDDHYSLDYWGQGTTLTVSS (SEQ ID NO: 671) or QVQLQQSGAELARPGASVKMSCKTSGYTFTRYTMHWVKQRPGQGLEWIGYINPSRGYTNY NQKFKDKATLTTDKSSSTAYMQLSSLTSEDSAVYYCARYYDDHYCLDYWGQGTTLTVSS (SEQ ID NO: 726). In some embodiments, the VL of the additional anti-CD3 binding domain comprises a sequence with at least 80% sequence identity to the sequence QIVLTQSPAIMSASPGEKVTMTCRASSSVSYMNWYQQKSGTSPKRWIYDTSKVASGVPYRF SGSGSGTSYSLTISSMEAEDAATYYCQQWSSNPLTFGAGTKLELK (SEQ ID NO: 672). In some embodiments, the additional anti-CD3 binding domain comprises a sequence with at least 80% sequence identity to QVQLQQSGAELARPGASVKMSCKTSGYTFTRYTMHWVKQRPGQGLEWIGYINPSRGYTNY NQKFKDKATLTTDKSSSTAYMQLSSLTSEDSAVYYCARYYDDHYSLDYWGQGTTLTVSSG GGGSGGGGSGGGGSGGGGSQIVLTQSPAIMSASPGEKVTMTCRASSSVSYMNWYQQKSGT SPKRWIYDTSKVASGVPYRFSGSGSGTSYSLTISSMEAEDAATYYCQQWSSNPLTFGAGTKL ELK (SEQ ID NO: 727) or QIVLTQSPAIMSASPGEKVTMTCRASSSVSYMNWYQQKSGTSPKRWIYDTSKVASGVPYRF SGSGSGTSYSLTISSMEAEDAATYYCQQWSSNPLTFGAGTKLELKGGGGSGGGGSGGGGSG GGGSGGGGSQVQLQQSGAELARPGASVKMSCKTSGYTFTRYTMHWVKQRPGQGLEWIGY INPSRGYTNYNQKFKDKATLTTDKSSSTAYMQLSSLTSEDSAVYYCARYYDDHYSLDYWG QGTTLTVSS (SEQ ID NO: 728). In some embodiments, the additional anti-CD3 binding domain comprises a sequence with at least 80% sequence identity to QVQLQQSGAELARPGASVKMSCKTSGYTFTRYTMHWVKQRPGQGLEWIGYINPSRGYTNY NQKFKDKATLTTDKSSSTAYMQLSSLTSEDSAVYYCARYYDDHYCLDYWGQGTTLTVSSG GGGSGGGGSGGGGSGGGGSQIVLTQSPAIMSASPGEKVTMTCRASSSVSYMNWYQQKSGT SPKRWIYDTSKVASGVPYRFSGSGSGTSYSLTISSMEAEDAATYYCQQWSSNPLTFGAGTKL ELK (SEQ ID NO: 725) or QIVLTQSPAIMSASPGEKVTMTCRASSSVSYMNWYQQKSGTSPKRWIYDTSKVASGVPYRF SGSGSGTSYSLTISSMEAEDAATYYCQQWSSNPLTFGAGTKLELKGGGGSGGGGSGGGGSG GGGSGGGGSQVQLQQSGAELARPGASVKMSCKTSGYTFTRYTMHWVKQRPGQGLEWIGY INPSRGYTNYNQKFKDKATLTTDKSSSTAYMQLSSLTSEDSAVYYCARYYDDHYCLDYWG QGTTLTVSS (SEQ ID NO: 728).

In one aspect, the T cell engager comprises an additional anti-CD3 binding domain comprising a heavy chain variable region (VH) that comprises a heavy chain complementarity determining region 3 (HC CDR3) sequence of ARSGYYGDSDWYFDV (SEQ ID NO: 731) or SGYYGDSDWYFDV (SEQ ID NO: 607). In some embodiments, the VH of the additional anti-CD3 binding domain comprises a HC CDR2 sequence of INPYKGVS (SEQ ID NO: 730) or LINPYKGVSTYNQKFKD (SEQ ID NO: 608). In some embodiments, the VH of the additional anti-CD3 binding domain comprises a HC CDR1 sequence of GYSFTGYT (SEQ ID NO: 729) or GYTMN (SEQ ID NO: 605). In some embodiments, the additional anti-CD3 binding domain comprises a light chain variable region (VL) that comprises a light chain complementarity determining region 3 (LC CDR3) sequence of QQGNTLPWT (SEQ ID NO: 610). In some embodiments, the VL of the additional anti-CD3 binding domain comprises a LC CDR2 sequence of YTS (SEQ ID NO: 733) or YTSRLES (SEQ ID NO: 609). In some embodiments, the VL of the additional anti-CD3 binding domain comprises a LC CDR1 sequence of QDIRNY (SEQ ID NO: 732) or RASQDIRNYLN (SEQ ID NO: 608). In some embodiments, the additional anti-CD3 binding domain comprises: a HC CDR1 sequence of GYSFTGYT (SEQ ID NO: 729), a HC CDR2 sequence of INPYKGVS (SEQ ID NO: 730), a HC CDR3 sequence of ARSGYYGDSDWYFDV (SEQ ID NO: 731), a LC CDR1 sequence of QDIRNY (SEQ ID NO: 732), a LC CDR2 sequence of YTS (SEQ ID NO: 733), and a LC CDR3 sequence of QQGNTLPWT (SEQ ID NO: 610).

In some embodiments, the additional anti-CD3 binding domain comprises: a HC CDR1 sequence of GYTMN (SEQ ID NO: 605), a HC CDR2 sequence of LINPYKGVSTYNQKFKD (SEQ ID NO: 606), a HC CDR3 sequence of SGYYGDSDWYFDV (SEQ ID NO: 607), a LC CDR1 sequence of RASQDIRNYLN (SEQ ID NO: 608), a LC CDR2 sequence of YTSRLES (SEQ ID NO: 609), and a LC CDR3 sequence of QQGNTLPWT (SEQ ID NO: 610).

In some embodiment, the VH of the additional anti-CD3 binding domain comprises a sequence with at least 80% sequence identity to the sequence EVQLVESGGGLVQPGGSLRLSCAASGYSFTGYTMNWVRQAPGKGLEWVALINPYKGVSTY NQKFKDRFTISVDKSKNTAYLQMNSLRAEDTAVYYCARSGYYGDSDWYFDVWGQGTLVT VSS (SEQ ID NO: 603). In some embodiments, the VL of the additional anti-CD3 binding domain comprises a sequence with at least 80% sequence identity to the sequence AIQMTQSPSSLSASVGDRVTITCRASQDIRNYLNWYQQKPGKAPKLLIYYTSRLESGVPSRFS GSGSGTDYTLTISSLQPEDFATYYCQQGNTLPWTFGQGTKVEIK (SEQ ID NO: 604). In some embodiments, the additional anti-CD3 binding domain comprises a sequence with at least 80% sequence identity to EVQLVESGGGLVQPGGSLRLSCAASGYSFTGYTMNWVRQAPGKGLEWVALINPYKGVSTY NQKFKDRFTISVDKSKNTAYLQMNSLRAEDTAVYYCARSGYYGDSDWYFDVWGQGTLVT VSSGGGGSGGGGSGGGGSGGGGSGGGSAIQMTQSPSSLSASVGDRVTITCRASQDIRNYLN WYQQKPGKAPKLLIYYTSRLESGVPSRFSGSGSGTDYTLTISSLQPEDFATYYCQQGNTLPW TFGQGTKVEIK (SEQ ID NO: 602). In some embodiments, the additional anti-CD3 binding domain comprises a sequence with at least 80% sequence identity to AIQMTQSPSSLSASVGDRVTITCRASQDIRNYLNWYQQKPGKAPKLLIYYTSRLESGVPSRFS GSGSGTDYTLTISSLQPEDFATYYCQQGNTLPWTFGQGTKVEIKGGGGSGGGGSGGGGSGG GGSGGGSEVQLVESGGGLVQPGGSLRLSCAASGYSFTGYTMNWVRQAPGKGLEWVALINP YKGVSTYNQKFKDRFTISVDKSKNTAYLQMNSLRAEDTAVYYCARSGYYGDSDWYFDVW GQGTLVTVSS (SEQ ID NO: 601).

In some embodiments, the T cell engager comprises an additional anti-CD3 binding domain comprising a heavy chain variable region (VH) that comprises a heavy chain complementarity determining region 3 (HC CDR3) sequence of AAKIRPYIFKIAGQYDY (SEQ ID NO: 736) or KIRPYIFKIAGQYDY (SEQ ID NO: 618). In some embodiments, the VH of the additional anti-CD3 binding domain comprises a HC CDR2 sequence of IVWSDGNT (SEQ ID NO: 735) or AIVWSDGNTYYEDFVKG (SEQ ID NO: 617). In some embodiments, the VH of the additional anti-CD3 binding domain comprises a HC CDR1 sequence of GRTYRGYS (SEQ ID NO: 734) or GYSMA (SEQ ID NO: 616). In some embodiments, the additional anti-CD3 binding domain is a VHH domain. In some embodiments, the additional anti-CD3 binding domain comprises: a HC CDR1 sequence of GRTYRGYS (SEQ ID NO: 734), a HC CDR2 sequence of IVWSDGNT (SEQ ID NO: 735), and a HC CDR3 sequence of AAKIRPYIFKIAGQYDY (SEQ ID NO: 736). In some embodiments, the additional anti-CD3 binding domain comprises: a HC CDR1 sequence of GYSMA (SEQ ID NO: 616), a HC CDR2 sequence of AIVWSDGNTYYEDFVKG (SEQ ID NO: 617), and a HC CDR3 sequence of KIRPYIFKIAGQYDY (SEQ ID NO: 618). In some embodiments, the VH of the additional anti-CD3 binding domain comprises a sequence with at least 80% sequence identity to the sequence EVQLVESGGGPVQAGGSLRLSCAASGRTYRGYSMAWFRQSPGKEREFVAAIVWSDGNTYY EDFVKGRFTISRDSAKNTLYLQMTNLKPEDTALYYCAAKIRPYIFKIAGQYDYWGQGTQVT VSS (SEQ ID NO: 615).

In one aspect, the T cell engager further comprises an anti-TRBC1 binding domain comprising a heavy chain variable region (VH) that comprises a heavy chain complementarity determining region 3 (HC CDR3) sequence of ARGAGYNFDGAYRFEDF (SEQ ID NO: 742) or GAGYNFDGAYRFEDF (SEQ ID NO: 739). In some embodiments, the VH of the anti-TRBC1 binding domain comprises a HC CDR2 sequence of INPYNDDI (SEQ ID NO: 741) or FINPYNDDIQSNERFRG (SEQ ID NO: 738). In some embodiments, the VH of the anti-TRBC1 binding domain comprises a HC CDR1 sequence of GYTFTGYV (SEQ ID NO: 740) or GYVMH (SEQ ID NO: 737). In some embodiments, the anti-TRBC1 binding domain comprises a light chain variable region (VL) that comprises a light chain complementarity determining region 3 (LC CDR3) sequence of SQSTHVPYT (SEQ ID NO: 745). In some embodiments, the VL of the anti-TRBC1 binding domain comprises a LC CDR2 sequence of RVS (SEQ ID NO: 747) or RVSNRFP (SEQ ID NO: 744). In some embodiments, the VL of the anti-TRBC1 binding domain comprises a LC CDR1 sequence of QRLVHSNGNTY (SEQ ID NO: 746) or RSSQRLVHSNGNTYLH (SEQ ID NO: 743).

In some embodiments, the anti-TRBC1 binding domain comprises: a HC CDR1 sequence of GYTFTGYV (SEQ ID NO: 740), a HC CDR2 sequence of INPYNDDI (SEQ ID NO: 741), a HC CDR3 sequence of ARGAGYNFDGAYRFEDF (SEQ ID NO: 742), a LC CDR1 sequence of QRLVHSNGNTY (SEQ ID NO: 746), a LC CDR2 sequence of RVS (SEQ ID NO: 747), and a LC CDR3 sequence of SQSTHVPYT (SEQ ID NO: 745). In one embodiment, the anti-TRBC1 binding domain comprises: a HC CDR1 sequence of GYVMH (SEQ ID NO: 737), a HC CDR2 sequence of FINPYNDDIQSNERFRG (SEQ ID NO: 738), a HC CDR3 sequence of GAGYNFDGAYRFEDF (SEQ ID NO: 739), a LC CDR1 sequence of RSSQRLVHSNGNTYLH (SEQ ID NO: 743), a LC CDR2 sequence of RVSNRFP (SEQ ID NO: 744), and a LC CDR3 sequence of SQSTHVPYT (SEQ ID NO: 745).

In one embodiment, the anti-TRBC1 binding domain (VH) comprises a sequence with at least 80% sequence identity to the sequence EVRLQQSGPDLIKPGASVKMSCKASGYTFTGYVMHWVYKQRPGQGLEWIGFINPYNDDIQ SNERFRGKATLTSDKSSTTAYMELSSLTSEDSAVYYCARGAGYNFDGAYRFEDFWGQGTT LTVSS (SEQ ID NO: 625). In one embodiment, the VL of the anti-TRBC1 binding domain comprises a sequence with at least 80% sequence identity to the sequence DVVMTQSPLSLPYSLGDQASISCRSSQRLVHSNGNTYLHWYLQKPGQSPKLLIYRVSNRFPG VPDRFSGSGSGTDFTLKISRVEAEDLGIYFCSQSTHVPYTFGGGTKLEIKR (SEQ ID NO: 626). In some embodiments, the anti-TRBC1 binding domain comprises a sequence with at least 80% sequence identity to EVRLQQSGPDLIKPGASVKMSCKASGYTFTGYVMHWVYKQRPGQGLEWIGFINPYNDDIQ SNERFRGKATLTSDKSSTTAYMELSSLTSEDSAVYYCARGAGYNFDGAYRFEDFWGQGTT LTVSSGGGGSGGGGSGGGGSDVVMTQSPLSLPYSLGDQASISCRSSQRLVHSNGNTYLHWY LQKPGQSPKLLIYRVSNRFPGVPDRFSGSGSGTDFTLKISRVEAEDLGIYFCSQSTHVPYTFG GGTKLEIKR (SEQ ID NO: 624).

In some embodiments, the anti-TRBC1 binding domain comprises a sequence with at least 80% sequence identity to DVVMTQSPLSLPYSLGDQASISCRSSQRLVHSNGNTYLHWYLQKPGQSPKLLIYRVSNRFPG VPDRFSGSGSGTDFTLKISRVEAEDLGIYFCSQSTHVPYTFGGGTKLEIKRGGGGSGGGGSG GGGSEVRLQQSGPDLIKPGASVKMSCKASGYTFTGYVMHWVYKQRPGQGLEWIGFINPYN DDIQSNERFRGKATLTSDKSSTTAYMELSSLTSEDSAVYYCARGAGYNFDGAYRFEDFWGQ GTTLTVSS (SEQ ID NO: 748).

In one aspect, the T cell engager further comprises an anti-CD2 binding domain comprising a heavy chain variable region (VH) that comprises a heavy chain complementarity determining region 3 (HC CDR3) sequence of AAVRDYVGMPYYSGSAYEY (SEQ ID NO: 749) or VRDYVGMPYYSGSAYEY (SEQ ID NO: 750). In some embodiments, the VH of the anti-CD2 binding domain comprises a HC CDR2 sequence of IRPGTIP (SEQ ID NO: 751) or AIRPGTIPYYSESVKG (SEQ ID NO: 752). In some embodiments, the VH of the anti-CD2 binding domain comprises a HC CDR1 sequence of GLTFSSYG (SEQ ID NO: 753) or SYGMA (SEQ ID NO: 754). In some embodiments, the anti-CD2 binding domain is a VHH domain. In some embodiments, the anti-CD2 binding domain comprises: a HC CDR1 sequence of GLTFSSYG (SEQ ID NO: 753), a HC CDR2 sequence of IRPGTIP (SEQ ID NO: 751), and a HC CDR3 sequence of AAVRDYVGMPYYSGSAYEY (SEQ ID NO: 749).

In some embodiments, the anti-CD2 binding domain comprises: a HC CDR1 sequence of SYGMA (SEQ ID NO: 754), a HC CDR2 sequence of AIRPGTIPYYSESVKG (SEQ ID NO: 752), and a HC CDR3 sequence of VRDYVGMPYYSGSAYEY (SEQ ID NO: 750).

In some embodiments, the VH of the anti-CD2 binding domain comprises a sequence with at least 80% sequence identity to the sequence QVQLVESGGGLVQAGGSLRLSCAASGLTFSSYGMAWFRRALGREREFVGAIRPGTIPYYSE SVKGRFTVSKDNAKNTVSLQMNSLKPEDTAVYYCAAVRDYVGMPYYSGSAYEYWGQGT QVTVSS (SEQ ID NO: 623).

In some embodiments, the one or more binding domains of the T cell engager is connected to the first polypeptide or to the second polypeptide.

In some embodiments, the first polypeptide further comprises a dimerizing domain fused to the TRAV domain, and the second polypeptide comprises a dimerizing domain fused to the TRBV domain.

In some embodiments, the first polypeptide comprises, from N to C terminus, the dimerizing domain of the first polypeptide, a peptide linker, and the TRAV domain, and the second polypeptide comprises, from N to C terminus, the dimerizing domain of the second polypeptide, a peptide linker, and the TRBV domain.

In some embodiments, the dimerizing domain of the first polypeptide comprises a TCR alpha constant (TRAC) domain or portion thereof; and the dimerizing domain of the second polypeptide comprises a TCR beta constant (TRBC) domain or portion thereof.

In some embodiments, the dimerizing domain of the first polypeptide and the dimerizing domain of the second polypeptide are linked by one or more disulfide bridges.

In some embodiments, the dimerizing domain of the first polypeptide and the dimerizing domain of the second polypeptide are linked by a single disulfide bridge.

In some embodiments, the first polypeptide comprises the T cell engager.

In some embodiments, the T cell engager is connected to the dimerization domain of the first polypeptide.

In some embodiments, the first polypeptide comprises, from N to C terminus, the T cell engager, the dimerization domain and the TRAV domain.

In some embodiments, the first polypeptide comprises, from N to C terminus, the TRAV domain and the T cell engager.

In some embodiments, the second polypeptide comprises the T cell engager.

In some embodiments, the second polypeptide comprises, from N to C terminus, the T cell engager, the dimerizing domain and the TRBV domain.

In some embodiments, the second polypeptide comprises, from N to C terminus, the TRBV domain and the T cell engager.

In some embodiments, the T cell engager comprises an scFv that binds to the receptor expressed by a T cell, and wherein the polypeptide comprises, from N to C terminus, the scFv, the dimerization domain and the TRAV domain.

In some embodiments, the T cell engager comprises an scFv that binds to the receptor expressed by a T cell and wherein the polypeptide comprises from N to C terminus, the dimerization domain, the TRAV domain and the scFv.

In some embodiments, the T cell engager comprises an scFv that binds to the receptor expressed by a T cell and wherein the polypeptide comprises, from N to C terminus, the scFv, the dimerization domain and the TRBV domain.

In some embodiments, the T cell engager comprises an scFv that binds to a receptor expressed by a T cell and wherein the polypeptide comprises from N to C terminus, the dimerization domain, the TRBV domain and the scFv.

In some embodiments, the T cell engager comprises a VHH that binds to a receptor expressed by a T cell and wherein the polypeptide comprises, from N to C terminus, the VHH, the dimerization domain and the TRAV domain.

In some embodiments, the T cell engager comprises a VHH that binds to a receptor expressed by a T cell and wherein the polypeptide comprises from N to C terminus, the dimerization domain, the TRAV domain and the VHH.

In some embodiments, the T cell engager comprises a VHH that binds to the receptor expressed by a T cell and wherein the polypeptide comprises, from N to C terminus, the VHH, the dimerization domain and the TRBV domain.

In some embodiments, the T cell engager comprises a VHH that binds to the receptor expressed by a T cell and wherein the polypeptide comprises from N to C terminus, the dimerization domain, the TRBV domain and the VHH.

In some embodiments, the T cell engager comprises a first VHH domain and a second VHH domain configured to bind to the same receptor expressed by a T cell.

In some embodiments, the T cell engager comprises a first VHH domain that binds the first receptor expressed by a T cell and the second VHH domain that binds the second receptor expressed by a T cell.

In some embodiments, the T cell engager comprises an scFv binding to a first T cell receptor and a VHH binding to a second T cell receptor, wherein the scFv and the VHH are connected by a peptide linker.

In some embodiments, the first polypeptide comprises the first binding domain of the T cell engager that binds to the extracellular domain of the first receptor expressed by a T cell; and the second polypeptide comprises the second binding domain of the T cell engager that binds to the extracellular domain of the second receptor expressed by the T cell.

In some embodiments, the first binding domain of the T cell engager is a first VHH that is fused to the first polypeptide and binds to CD3; and the second binding domain of the T cell engager is a second VHH fused to the second polypeptide and binds to CD2.

In some embodiments, the first binding domain of the T cell engager is a first VHH that binds to CD2; and the second binding domain of the T cell engager is a second VHH that binds to CD3.

In some embodiments, the T cell engager is fused with the first or second polypeptide via a peptide linker.

In one aspect, provided herein is a recombinant nucleic acid comprising a sequence encoding a multispecific molecule, wherein the multispecific molecule comprises (i) an MHC-peptide complex engager comprising: (a) a T cell receptor (TCR) alpha variable (TRAV) domain and a TCR alpha constant (TRAC) domain, and (b) a TCR beta variable (TRBV) domain and a TCR beta constant (TRBC) domain; and (ii) a T cell engager comprising one or more binding domains that bind to an extracellular domain of a receptor expressed by a T cell, wherein the MHC-peptide complex engager and the T cell engager form a continuous single polypeptide chain comprising, from N-terminus to C-terminus, the one or more binding domains of the T cell engager, the TRBV domain, the TRBC domain, the TRAV domain, and the TRAC domain. In some embodiments, the multispecific molecule further comprises a linker connecting the TRBC domain and the TRAV domain. In some embodiments, the linker is a non-cleavable linker. In some embodiments, the linker comprises 20-38 amino acid sequences. In some embodiments, the linker is a structure guided flexible linker. In some embodiments, the linker comprises Gly, Ser, Ala and/or Glu residues.

In some embodiments, the multispecific molecule comprises a sequence of SEQ ID NO: 862 without a signal peptide of SEQ ID NO: 668. In some embodiments, the multispecific molecule comprises one or more sequences of SEQ ID NO: 604, SEQ ID NO: 682, SEQ ID NO: 603, (SEQ ID NO: 683) (GGGGS), SEQ ID NO: 620, SEQ ID NO: 863, SEQ ID NO: 864, SEQ ID NO: 619, SEQ ID NO: 865, and SEQ ID NO: 866. In some embodiments, the multispecific molecule comprises a sequence of SEQ ID NO: 862. In some embodiments, the multispecific molecule comprises a sequence of SEQ ID NO: 862 without a signal peptide of SEQ ID NO: 668.

In one aspect, provided herein is a recombinant nucleic acid comprising a sequence encoding a multispecific molecule, wherein the multispecific molecule comprises (i) an MHC-peptide complex engager comprising: (a) a T cell receptor (TCR) alpha variable (TRAV) domain and a TCR alpha constant (TRAC) domain, and (b) a TCR beta variable (TRBV) domain and a TCR beta constant (TRBC) domain; and (ii) a T cell engager comprising one or more binding domains that bind to an extracellular domain of a receptor expressed by a T cell, wherein the MHC-peptide complex engager and the T cell engager form a continuous single polypeptide chain comprising, from N-terminus to C-terminus, the one or more binding domains of the T cell engager, the TRAV domain, the TRAC domain, the TRBV domain, and the TRBC domain. some embodiments, the multispecific molecule further comprises a linker connecting the TRAC domain and the TRBV domain. In some embodiments, the linker comprises between 20-38 amino acids, comprising Gly, Ser, Ala and/or Glu amino acids. In some embodiments, the one or more binding domains of the T cell engager comprises a single chain variable fragment (scFv). In some embodiments, the scFv comprises, from N-terminus to C-terminus, a light chain variable domain (VL) followed by a heavy chain variable domain (VH). In some embodiments, the scFv comprises, from N-terminus to C-terminus, a heavy chain variable domain (VH) followed by a light chain variable domain (VL).

In one aspect, provided herein is a recombinant nucleic acid comprising a sequence encoding a multispecific molecule, wherein the multispecific molecule comprises (i) an MHC-peptide complex engager comprising: (a) a T cell receptor (TCR) alpha variable (TRAV) domain and a TCR alpha constant (TRAC) domain, and (b) a TCR beta variable (TRBV) domain and a TCR beta constant (TRBC) domain; and (ii) a T cell engager comprising one or more binding domains that bind to an extracellular domain of a receptor expressed by a T cell, wherein the MHC-peptide complex engager and the T cell engager form a continuous single polypeptide chain comprising, from N-terminus to C-terminus, the TRAV domain, the TRAC domain, the one or more binding domains of the T cell engager, the TRBV domain, and the TRBC domain. In some embodiments, the multispecific molecule further comprises a linker connecting the TRAC domain and the one or more binding domains of the T cell engager. In some embodiments, the one or more binding domains of the T cell engager comprises a single chain variable fragment (scFv). In some embodiments, the scFv comprises, from N-terminus to C-terminus, a light chain variable domain (VL) followed by a heavy chain variable domain (VH). In some embodiments, the scFv comprises, from N-terminus to C-terminus, a heavy chain variable domain (VH) followed by a light chain variable domain (VL).

In some embodiments, the multispecific molecule comprises one or more sequences selected from the group consisting of SEQ ID NO: 619, SEQ ID NO: 868, SEQ ID NO: 869, SEQ ID NO: 604, SEQ ID NO: 870, SEQ ID NO: 603, SEQ ID NO:620, SEQ ID NO: 871, and SEQ ID NO: 866. In some embodiments, the multispecific molecule comprises a sequence of SEQ ID NO: 867. In some embodiments, the multispecific molecule comprises a sequence of SEQ ID NO: 867 without a signal peptide of SEQ ID NO: 667.

In one aspect, provided herein is a recombinant nucleic acid comprising a sequence encoding a multispecific molecule, wherein the multispecific molecule comprises: (i) an MHC-peptide complex engager comprising: (a) a T cell receptor (TCR) alpha variable (TRAV) domain and a TCR alpha constant (TRAC) domain, and (b) a TCR beta variable (TRBV) domain and a TCR beta constant (TRBC) domain; and (ii) a T cell engager comprising one or more binding domains that bind to an extracellular domain of a receptor expressed by a T cell, wherein the MHC-peptide complex engager and the T cell engager form a continuous single polypeptide chain comprising, from N-terminus to C-terminus, the TRBV domain, the TRBC domain, the one or more binding domains of the T cell engager, the TRAV domain, and the TRAC domain. In some embodiments, the multispecific molecule further comprises a linker connecting the TRBC domain and the one or more binding domains of the T cell engager, wherein the linker comprises 20-37 amino acids and comprises Gly, Ser, Ala and/or Glu amino acids.

In one aspect, provided herein is a recombinant nucleic acid comprising a sequence encoding a multispecific molecule, wherein the multispecific molecule comprises (i) an MHC-peptide complex engager comprising: (a) a T cell receptor (TCR) alpha variable (TRAV) domain, and (b) a TCR beta variable (TRBV) domain; and (ii) a T cell engager comprising a single chain variable fragment (scFv) that bind to an extracellular domain of a receptor expressed by a T cell, wherein the MHC-peptide complex engager and the T cell engager form a continuous single polypeptide chain comprising, from N-terminus to C-terminus, the scFv, the TRBV domain, and the TRAV domain. In some embodiments, the recombinant nucleic acid further comprises a TCR alpha constant (TRAC) domain. In some embodiments, the recombinant nucleic acid further comprises a TCR beta constant (TRBC) domain. In some embodiments, the T cell engager form a continuous single polypeptide chain comprising, from N-terminus to C-terminus, the scFv, the TRBV domain, the TRBC domain, the TRAV domain and the TRAC domain. In some embodiments, the recombinant nucleic acid further the T cell engager comprises a VL domain and a VH domain from N-terminus to C-terminus. In some embodiments, the recombinant nucleic acid further the T cell engager comprises a VH domain and a VL domain from N-terminus to C-terminus.

In one aspect, provided herein is a recombinant nucleic acid comprising a sequence encoding a multispecific molecule, wherein the multispecific molecule comprises (i) an MHC-peptide complex engager comprising: (a) a T cell receptor (TCR) alpha variable (TRAV) domain, and (b) a TCR beta variable (TRBV) domain; and (ii) a T cell engager comprising a single chain variable fragment (scFv) that bind to an extracellular domain of a receptor expressed by a T cell, wherein the MHC-peptide complex engager and the T cell engager form a continuous single polypeptide chain comprising, from N-terminus to C-terminus, the scFv, the TRAV domain, and the TRBV domain.

In some embodiments the recombinant nucleic acid further comprises a TCR alpha constant (TRAC) domain. In some embodiments the recombinant nucleic acid further comprises a TCR beta constant (TRBC) domain. In some embodiments, the T cell engager form a continuous single polypeptide chain comprising, from N-terminus to C-terminus, the scFv, the TRBV domain, the TRBC domain, the TRAV domain and the TRAC domain. In some embodiments, the T cell engager comprises a VL domain and a VH domain from N-terminus to C-terminus. In some embodiments, the T cell engager comprises a VH domain and a VL domain from N-terminus to C-terminus.

In one aspect, provided herein is a recombinant nucleic acid comprising a sequence encoding a multispecific molecule, wherein the multispecific molecule comprises (i) an MHC-peptide complex engager comprising: (a) a T cell receptor (TCR) alpha variable (TRAV) domain, and (b) a TCR beta variable (TRBV) domain; and (ii) a T cell engager comprising a single chain variable fragment (scFv) that bind to an extracellular domain of a receptor expressed by a T cell, wherein the MHC-peptide complex engager and the T cell engager form a continuous single polypeptide chain comprising, from N-terminus to C-terminus, the TRAV domain, the scFv, and the TRBV domain. In some embodiments the recombinant nucleic acid further comprises a TCR alpha constant (TRAC) domain. In some embodiments the recombinant nucleic acid further comprises a TCR beta constant (TRBC) domain.

In some embodiments, the T cell engager form a continuous single polypeptide chain comprising, from N-terminus to C-terminus, the scFv, the TRAV domain, the TRAC domain, the TRBV domain and the TRBC domain. In some embodiments, the T cell engager comprises a VL domain and a VH domain from N-terminus to C-terminus. In some embodiments, the T cell engager comprises a VH domain and a VL domain from N-terminus to C-terminus.

In one aspect, provided herein is a recombinant nucleic acid comprising a sequence encoding a multispecific molecule, wherein the multispecific molecule comprises (i) an MHC-peptide complex engager comprising: (a) a T cell receptor (TCR) alpha variable (TRAV) domain, and (b) a TCR beta variable (TRBV) domain; and (ii) a T cell engager comprising a single chain variable fragment (scFv) that bind to an extracellular domain of a receptor expressed by a T cell, wherein the MHC-peptide complex engager and the T cell engager form a continuous single polypeptide chain comprising, from N-terminus to C-terminus, the TRBV domain, the scFv, and the TRAV domain. In some embodiments, the recombinant nucleic acid further comprises a TCR alpha constant (TRAC) domain. In some embodiments, the recombinant nucleic acid further comprises a TCR beta constant (TRBC) domain. In some embodiments, the T cell engager form a continuous single polypeptide chain comprising, from N-terminus to C-terminus, the scFv, the TRAV domain, the TRAC domain, the TRBV domain and the TRBC domain. In some embodiments, the T cell engager comprises a VL domain and a VH domain from N-terminus to C-terminus. In some embodiments, the T cell engager comprises a VH domain and a VL domain from N-terminus to C-terminus.

In some embodiments, the recombinant nucleic acid further encodes a peptide linker, wherein the linker comprises an amino acid sequence having 90% sequence identity with the sequence of SEQ ID NO: 864, or 872 or 873. In some embodiments, the TRAC domain comprises a wildtype murine TRAC sequence or a wildtype human TRAC sequence. In some embodiments, the TRAC domain does not comprise a mutation. In some embodiments, the TRAC domain comprises a mutation compared to a murine TRAC sequence or a wildtype human TRAC sequence. In some embodiments, the mutation is a stability enhancing mutation. In some embodiments, the mutation is selected from the group consisting of S139F, T150I and A190T, the residue positions are numbered based on Kabat numbering scheme.

In some embodiments, the TRBC domain comprises a wildtype murine TRBC sequence or a wildtype human TRBC sequence. In some embodiments, the TRBC domain does not comprise a mutation compared to the wildtype sequence. In some embodiments, the TRBC domain comprises a mutation compared to a murine TRBC sequence or a wildtype human TRBC sequence. In some embodiments, the mutation is a stability enhancing mutation. In some embodiments, the mutation is selected from the group consisting of E134K, H139R, D155P and S170D, the residue positions are numbered based on Kabat numbering scheme.

In one aspect, provided herein is a composition comprising the multispecific molecule encoded by the sequence of the recombinant nucleic acid of the composition of any one of the embodiments described above.

In some embodiments, the multispecific molecule is isolated or purified.

Provided herein is a pharmaceutical composition comprising the composition of any one of the embodiments described above.

In one aspect, provided herein is a method of treating cancer in a subject in need thereof comprising administering a therapeutically effective amount of the pharmaceutical described above.

In one aspect, provided herein is a method of making a multispecific molecule, wherein the multispecific molecule is an engineered T cell receptor (TCR) construct comprising: (i) an MHC-peptide complex engager comprising: (a) a T cell receptor (TCR) alpha variable (TRAV) domain, and (b) a TCR beta variable (TRBV) domain; and (ii) a T cell engager comprising one or more binding domains that bind to an extracellular domain of a receptor expressed by a T cell, wherein the multispecific molecule comprises a post-translational modification; wherein the method comprises expressing the multispecific molecule from a recombinant nucleic acid comprising a sequence encoding the multispecific molecule in a mammalian cell, thereby producing a multispecific molecule comprising a mammalian post-translational modification signature. In one embodiment, the method further comprises isolating or purifying the multispecific molecule comprising the mammalian post-translational modification signature. In some embodiments, the cell secretes the first polypeptide and the second polypeptide encoded by the sequence of the recombinant nucleic acid of the composition described above.

Provided herein is a mammalian cell comprising the recombinant nucleic acid of the composition of any one of the embodiments described above.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWING

The salient features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.” herein), of which:

FIG. 1A is a schematic that illustrates in word diagram an overview of the work undertaken.

FIG. 1B shows a schematic diagram of a plasmid, expression and tetramer binding assay. SP, signal peptide. Flow cytometry scan is for illustration purpose only and is not data.

FIG. 1C shows expression of TCR library, enrichment and tetramer assay.

FIG. 1D shows a schematic tetramer and monomer assays.

FIG. 2 is a schematic that shows a general overview of various experimental bispecific sTCR scaffold designs.
At center, a schematic diagram shows a p-MHC complex presented by a tumor cell on the top, a multispecific engager (soluble TCR) on the left, comprising TRAV and TRBV domains (an MHC-peptide complex engager) for association with the p-MHC, graphically indicated by the bidirectional arrow. The sTCR TRAC and a TRBC domains form a scaffold, that are interconnected by disulfide bonds at positions 1 and 2 as marked on the schematic diagrams. The T cell engager comprises an anti-CD3 scFv, capable to binding CD3, a surface molecule on a T cell, and is attached to a variable domain via a short peptide linker.
Top left, a bispecific sTCR as described above, with a CD3 specific scFv as T cell engager attached to TRBV.
Bottom left, similar to top left, but with the CD3 specific scFv as T cell engager attached to TRAV.
Top right, a bispecific sTCR as described above, with a CD3 specific scFv as T cell engager attached to TRBC.
Bottom right, a bispecific sTCR as described above, with a CD3 specific scFv as T cell engager attached to TRAC.

FIG. 3 features schematic representations of ten different sTCR scaffold structures constructed and tested. Scaffold 2, TRAV-TRBV make up the p-MHC engager, is the basic structure for evaluating stability, with purification tag 10×HIS and affinity acceptor molecule Biotin Acceptor Protein (BAP) tag. Positions 1 and 2 indicate inter-chain disulfide linkages. Scaffold 5 illustrates two sets of TCRs being connected to IgG domains. Each sTCR comprises two chains—an alpha chain comprising TRAV-TRAC; and a beta chain comprising a TRBV and TRBC. Scaffold 6 comprises a TRAV and a TRAC that are stabilized by two scaffolding polypeptides having one inter-chain disulfide bond. The two scaffolding polypeptides are immunoglobulin Fv domains arranged in an Fv clasp. Scaffold 14 comprises the sTCR fused to a immunoglobulin (Ig) Fc structure. The Fc portions are engineered and comprise knob-and-hole configurations for increased stability in bound form. Scaffold 19 comprises a tagged sTCR molecule comprising the TRAV-TRAC-TRBV-TRBC tetrameric structure, fused to an anti-CD3 scFv via a short linker peptide. Scaffold 20 has a basic structural similarity to Scaffold 6, and in addition comprises an scFv attached to TRBV via a short linker peptide. Scaffold 21 comprises the basic structure similar to Scaffold 14, and further comprises an scFv attached to TRBV via a short linker peptide. Scaffold 36 comprises the basic sTCR configuration of Scaffold 2 and is linked to two scFvs in tandem attached to TRBV via peptide linkers. Scaffold 37 comprises a basic tetrameric sTCR configuration, where the TRAC and TRBC are attached tags BAP, Sortase A and 10×HIS. Scaffold 43 comprises the basic tetrameric sTCR structure connected to Ig Fc domains. Arrow points to protease target sites that are used for cleavage of the sTCR from the Ig domains.

FIG. 4 upper panel shows a diagrammatic representation of the experimental protocol involving transfection of the constructs and purification. A representative gel filtration result for verification of purity of the product is shown at upper right. Lower panel indicates the workflow and timeline of tumor cell killing by effector T cells in an in vitro assay.

FIGS. 5A and 5B shows an exemplary representative data from generation and purification of Scaffold 2, showing gel electrophoresis of the purified sTCRs from cell culture media (FIG. 5A), and single protein peak at mid-elusion (FIG. 5B) respectively.

FIG. 6 shows a schematic layout of the workflow for target cell cytotoxicity assay, for testing the functional efficacy of the soluble TCR constructs. T cells were obtained from peripheral blood samples of healthy donor. A375 cells transfected with polynucleotides encoding the peptide and the MHC, and expressing the polypeptides were used as target cells. Purified sTCR products were added to culture at the concentrations indicated for assaying T cell activation and cytotoxicity, measured by loss of GFP positive target cells compared to untreated control.

FIG. 7A shows structural design of Scaffold 19 (Ros9a Scaffold 19) construct having TCR alpha and beta variable domains capable of binding mutated RAS epitope bound to MHC encoded by HLA-C:08:02.

FIG. 7B shows cytotoxicity data using the method outlined in the workflow layout of FIG. 6. Cytotoxicity was measured by the area with total number of GFP-positive cells (=total green objects), which gives a measure of live cells. Accordingly higher cytotoxicity correlates with lower green cells.

FIG. 8A shows structural design of a Scaffold 20b (Ros9a Scaffold 20b) construct having TCR alpha and beta variable domains capable of binding mutated RAS epitope bound to MHC encoded by HLA-C:08:02. FIG. 8B shows cytotoxicity data.

FIG. 9A shows structural design of a Scaffold 21 (Ros9a Scaffold 21) construct having TCR alpha and beta variable domains capable of binding mutated RAS epitope bound to MHC encoded by HLA-C:08:02. FIG. 9B shows cytotoxicity data.

FIG. 10A shows structural design of Scaffold 19 (Ros9d Scaffold 19) construct having TCR alpha and beta variable domains capable of binding mutated RAS epitope bound to MHC encoded by HLA-C:08:02. FIG. 10B shows cytotoxicity data.

FIG. 11A shows structural design of Scaffold 21 (Ros9d Scaffold 21) construct having TCR alpha and beta variable domains capable of binding mutated RAS epitope bound to MHC encoded by HLA-C:08:02. FIG. 11B shows cytotoxicity data.

FIG. 12A shows structural design of Scaffold 19 (Ros10 Scaffold 19) construct having TCR alpha and beta variable domains capable of binding mutated RAS epitope bound to MHC encoded by HLA-C:08:02. FIG. 12B shows cytotoxicity data.

FIG. 13 shows a schematic layout of the workflow for target cell cytotoxicity assay, for testing the functional efficacy of the soluble TCR constructs. T cells were obtained from peripheral blood samples of healthy donor. Target cells are tumor cells of HPAC cell line which endogenously express the mutated Ras epitope and the HLA-C:08:02. Purified sTCR products were added to culture at the concentrations indicated for assaying T cell activation and cytotoxicity, measured by apoptosis marker Annexin V and Caspase 3/7 positive target cells compared to untreated control.

FIGS. 14A and 14B show imaging (FIG. 14A) and quantitative data (FIG. 14B) from the imaging assay of a comparison between Ros9a Scaffold 19 and Ros 10 scaffold 19 treatments showing annexin positive cells (orange) and caspase 3/7 positive cells (green) at different peptide concentrations. FIG. 14C shows imaging with only Annexin V positive cells.

FIGS. 15A-15B show cytotoxicity assay with donor's T cells that were rested for shorter period, showing increase in target cell cytotoxicity.

FIG. 16 illustrates schematic representations of various multispecific engager scaffolds having an anti-CD3 scFv and an additional T cell engager, comprising a CD8a ectodomain.

FIG. 17 illustrates schematic representations of various multispecific engager scaffolds having an anti-CD3 or anti-CD2 VHH domains. The amino acid sequences of Scaffolds 53, 55 and 57 are disclosed elsewhere in the specification, e.g. Table 10.

FIG. 18A-18B illustrates schematic representations of various multispecific engager scaffolds designed for testing. are fused with the basic tetrameric sTCR structure via short peptide linkers.
Scaffolds 45-48 contain a basic tetrameric structure in which TRAV and TRBV are linked to immunoglobulin constant regions. Heteromeric Igs are included for the two chains. Scaffolds 46 and 48 comprise disulfide bonds to present at position 2, which is omitted in Scaffolds 46 and 48.
Scaffold 49-51 was designed to include anti-CD3 scFv attached to TRAV or TRBV. In Scaffold 50, TRAV and TRBV are initially linked but comprises a cleavage sequence (F2A). Scaffold 49 comprises two polypeptides, the first polypeptide has the following orientation NH2-aCD3scFv_VH-TRAV-(linker+tag)-COOH, where aCD3scFv stands for antiCD3scFv; the second polypeptide has the following orientation NH2-TRBV-aCD3scFv_VL-(linker+tag)-COOH. Scaffold 50 is generated as a single polypeptide with a cleavable sequence (F2A) in between: NH2-aCD3scFv_VH-TRAV-(F2A)-TRBV-aCD3scFv_VL-(linker+tag)-COOH. Scaffold 51 is a single polypeptide with a linker in connecting the two juxtaposition chains: NH2-aCD3scFv_VH-TRAV-(linker)-TRBV-aCD3scFv_VL-(linker+tag)-COOH. Scaffold 52 is a single polypeptide with a linker in connecting the two juxtaposition chains: NH2-TRAV-aCD3scFv_VH-(linker)-aCD3scFv_VL-TRBV-(linker+tag)-COOH.

FIG. 19 illustrates schematic representations of various multispecific engager scaffolds designed for testing. Scaffolds 103, 106 and 137, 108 and 138 comprises the basic tetrameric sTCR structure. Of these, Scaffold 138 has an additional domain that binds TRBC1, in addition to an anti-CD3VHH. Scaffolds 139 and 142 are newer experimental designs that do not contain the basic tetrameric sTCR structure. In Scaffolds 139, each of the TRAV and TRBV are linked via a short peptide linker to an anti-CD3 VHH. Scaffold 142 comprises an anti-CD3 VHH domain attached to one of TRAV and TRBV, and an anti-TRBC1 scFv attached to the other of TRAV and TRBV via short peptide linkers.

FIG. 20 illustrates schematic representations of various multispecific engager scaffolds designed for testing.

FIG. 21 illustrates schematic representations of a multispecific engager scaffolds having one or more VHH T cell engagers. In these scaffold designs, the linker between the VHH and the TCR is an extended linker.

FIG. 22 shows a schematic layout of the workflow for target cell cytotoxicity assay for testing the functional efficacy of the soluble TCR constructs built on Scaffold 22. T cells were obtained from peripheral blood samples of healthy donor. A375 cells transfected with polynucleotides encoding the peptide and the MHC, and expressing the polypeptides were used as target cells. Purified sTCR products were added to culture at the concentrations indicated for assaying T cell activation and cytotoxicity, measured by loss of GFP positive target cells compared to untreated control.

FIG. 23 shows the structure and assay results to test the cytotoxic potential of the construct Ros9a Scaffold 22. The anti-CD3 arm is based on TR66-VHVL construct.

FIG. 24 shows the structure and assay results to test the cytotoxic potential of the construct Ros9a Scaffold 22. The anti-CD3 arm is based on TR66-VLVH construct.

FIG. 25 shows the structure and assay results to test the cytotoxic potential of the construct Ros9a Scaffold 22. The anti-CD3 arm is based on 210112-VHVL construct.

FIG. 26 shows the structure and assay results to test the cytotoxic potential of the construct Ros9a Scaffold 22. The anti-CD3 arm is based on 210112-VLVH construct.

FIG. 27 shows the structure and assay results to test the cytotoxic potential of the construct Ros9d Scaffold 22. The anti-CD3 arm is based on TR66-VHVL construct.

FIG. 28 shows the structure and assay results to test the cytotoxic potential of the construct Ros9d Scaffold 22. The anti-CD3 arm is based on 210112-VHVL construct.

FIG. 29 shows a schematic diagram of the workflow for assaying cytotoxic potential of the sTCR construct against tumor cell lines with endogenous KRAS G12D, an assay similar to as shown in FIG. 13.

FIG. 30 shows imaging of target HPAC tumor cells treated with Ros9a Scaffold 22 sTCR construct in presence of T cells, showing annexin positive cells (orange) and caspase 3/7 positive target apoptotic cells (green) at different peptide concentrations.

FIG. 31 shows quantitative assessment results from the analysis of cytotoxicity assay of FIG. 30.

FIG. 32 shows results from imaging analysis when target cells in the assay were stained from Annexin V alone.

FIG. 33 shows an assay similar to that shown in FIGS. 15A and 15B. The donor's T cells that were rested for shorter period than in the protocol for FIGS. 30-32, and this improves target cell cytotoxicity.

FIG. 34 shows a graph representing the quantitative assessments of the assay results shown in FIG. 33.

FIG. 35A shows graphical representation of structures of single chain soluble bispecific TCR (BiTE) constructs designed to enable mRNA formatting and delivery, and simplifying the development pipeline. In this design, a TRBC is connected to a TRAV fragment forming a single polypeptide chain comprising both TCR alpha and beta chains with another linker connecting an anti-CD3 scFv. The general framework of the structures are based on primary scaffold structures described herein. In some cases, as shown in the Figure, the linker between the TRBC and TRAV is a GS linker, (G4S)₇. Top left: Scaffold Name: Ros9a_SC107βα_antiCD3. Graphical representation of a construct having one or more point mutations in the constant (TRAC and TRBC) regions, that enhance the stability of the polypeptide. Additional mutations are designed to remove glycosylation sites. Top right: Scaffold name Ros9a_SC107wβα_antiCD3. Graphical representation of a construct that is devoid of several mutations in the constant domains for stability enhancing purpose as shown in the top left model. AlphaFold 3D modeling of the protein structure depicting spatial orientation of the domains of the polypeptide is shown at the bottom. Nt, N terminus; Ct, C terminus. The designs leave an anti-CD3 scFv N terminal end free. The TCR has an N terminal to C terminal orientation of its alpha and beta chains such that beta chain is N terminal to the alpha chain.

FIG. 35B shows schematic diagrams for single chain soluble TCR designs, Top left, Scaffold name: Ros9a_SC111αβ_antiCD3, with stabilizing mutations in the constant domain; Top right Scaffold name: Ros9a_SC111wαβ_antiCD3, and does not contain the stability enhancing constant domain mutations. AlphaFold model image at the bottom, in which a linker (such as (G₄S)₄) connects the N terminal end of the anti-CD3 scFv with the C terminal end of TRBC, forming the single chain with an internal ant-CD3 scFv, which access the same position as in the construct shown in FIG. 35A, but not constrained by the linker. In this case the alpha chain is N terminal to the beta chain.

FIG. 36 (top) shows a construct design of soluble BiTE (e.g., an mRNA encoding the polypeptide) having domain layout from N terminus to C terminus, N terminal anti-CD3 scFv comprising variable light chain (VL) and variable heavy chain (VH) domains, followed by a linker connecting the scFv with TCR beta chain comprising TRBV fused to TRBC, which is connected by a second longer linker to TCR alpha chain comprising TRAV fused to TRAC. Exemplary construct, Ros9a_SC107wβα_antiCD3. An exemplary construct Ros9a_SC107wβα_antiCD3 may have a sequence of SEQ ID NO: 862. SEQ ID NO: 862 lacks stability enhancing mutations S>F, T>I and A>T in TCR alpha contact (TRAC) region as shown in Table 12. FIG. 36 (bottom) shows a schematic of the structure of the polypeptide. Dotted line denotes the linker creating the single chain.

FIG. 37 (top) shows a soluble BiTE construct design with layout of domains from N terminus to C terminus, e.g., an mRNA encoding the specific domains: N terminal TCR alpha chain comprising TRAV fused to TRAC, followed by a linker connecting the TCR alpha with anti-CD3 scFv comprising variable light chain (VL) and variable heavy chain (VH) domains, followed by a linker connecting the scFv with TCR beta chain comprising TRBV fused to TRBC. Exemplary Construct Ros9a_SC111wαβ_antiCD3, FIG. 37 (bottom) shows a schematic structure of the polypeptide.

FIG. 38 (left) shows design and exemplary AlphaFold 3D modeling structure of a single polypeptide chain sTCR having a peptide linker connecting the TCR alpha and beta chains. Different linkers, H1 (linker 1), H2 (Linker 2) or GS linker (35GS) were tested, and for polypeptides in which the alpha and beta chain are in an alpha-beta as well as in a beta-alpha orientation from N terminus to C terminus. Cell surface expression and peptide binding assays for each construct were tested. Results shown in the graph at the right side.

FIG. 39A shows a schematic protocol for assaying the cytotoxic potential of the BiTE sTCRs. Single polypeptide chains having TCR alpha and beta domains and anti-CD3 scFv were cocultured with T cells (effectors) and target cancer cell lines (e.g., A375 cells) and cell death of the target cells were measured. PBMCs from three healthy donors were isolated, rested for one day in non-activating media before assay. In alternate assay set ups, shorter rest (3-5 hrs) were also tested. In alternative sets, T cells were isolated from PBMCs by negative selection (“untouched” purified T cells) and the assay was performed. (E:T, effector cell to target cell ratio).

FIG. 39B shows data depicting comparison of two sTCR BiTE constructs, one with Fc fusion at the TCR constant domains (top figure), the other without Fc fusion (bottom figure). E, CD3+(effector) cells expanded and O/N rested; T, (target cancer cell line) A375 transduced to express KRAS G12D+HLA C*08:02. The BiTE is a Ros9a sTCR based construct, added to the culture at a concentration ranging between 100 nM-0.4 nM. The data shows substantially less cytotoxic potential of the Fc fused construct.

FIG. 40 shows data comparing efficacy of the indicated single chain sTCR constructs versus a double chain sTCR construct in promoting T cell mediated target cell cytotoxicity. Upper left, T cell cytotoxicity data using an sTCR construct Ros9aS74_antiCD3, which comprises two separate TCR chains (alpha and beta chains); and which is shown as a control for assessing cytotoxic potential of single chain constructs designed herein. For example, Upper right, cytotoxicity data using single chain sTCR construct Ros9a_SC107_antiCD3, inset showing graphical view of Ros9a_SC107_antiCD3 polypeptide structure. Data shows Ros9a_SC107_antiCD3 has equivalent potency to Ros9aS74_antiCD3. Similar assays were used to assess cytotoxicity potential of single chain sTCR construct Ros9a_SC111_antiCD3 (Lower left), which had slightly lower potential for inducing cytotoxicity than Ros9aS74_antiCD3. However higher doses had considerable improvement on inducing cytotoxicity; and Lower right, data on sTCR Ros9a_SC113_antiCD3 construct. The effects were dose dependent, which is indicated in the index at the right hand column. Target cells express green fluorescence protein, reduction of which as a function of time was a fast and easy measure of the T cell mediated cytotoxicity.

FIG. 41 shows imaging data of target cell depletion in presence of the sTCR BiTE constructs in an assay as shown in FIG. 40.

### DETAILED DESCRIPTION

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) may be inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, is meant to encompass variations of +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

An “agent” can include any type of molecule and includes, but is not limited to, an antibody, a peptide, a protein, a polynucleotide (e.g., an oligonucleotide, RNA, or DNA), a small molecule, derivatives thereof and analogs thereof.

A “biologic sample” may be any tissue, cell, fluid, or other material derived from an organism. As used herein, the term “sample” may include a biologic sample such as any tissue, cell, fluid, or other material derived from an organism. The term “biological sample” may encompass a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

“Specifically binds” may refer to a condition in which a compound (e.g., peptide) recognizes and binds to a molecule (e.g., peptide or polypeptide), but does not substantially recognize and bind other molecules in a sample, for example, a biological sample, that is, the compound exhibits a selective binding to a molecule. A “binder” as described herein includes, but is not limited to, a protein, a polypeptide or fragments thereof, that exhibits specific binding to a cognate molecule. A binder may refer to an antigen binding domain, such as the first binding domain of a bispecific or trispecific engager, or the second antigen binding domain of a bispecific or trispecific engager, and so on. In some cases, a binder may be any biomolecule or fragment thereof, such as a peptide or conjugated peptide or a ligand that can specifically bind to a receptor on a cell and therefore exhibits specific binding of one portion of an exemplary engager.

In several cases, an “immune response” may include T cell mediated and/or B cell mediated immune responses that are influenced by modulation of T cell co-stimulation. Exemplary immune responses include T cell responses, e.g., cytokine production, and cellular cytotoxicity. In addition, the term immune response includes immune responses that are indirectly affected by T cell activation, e.g., antibody production (humoral responses) and activation of cytokine responsive cells.

A “functional derivative” of a native sequence polypeptide may be a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. The term “derivative” may encompass both amino acid sequence variants of polypeptide and covalent modifications thereof.

In some cases, a “binder” may refer to a binding domain in a recombinant polypeptide generated by design, as described herein. A binding domain sequence may be derived from a naturally occurring protein and engineered into the recombinant polypeptide by recombinant DNA technology. A binding domain may be selected on the basis of its binding specificity to its target or cognate element. A binding domain may be derived from a protein that is an antibody or a functional fragment thereof, that binds to the target antigen or the cognate molecule. A desired characteristic of a binding domain may be high specificity, high binding affinity or both, towards its target. A binding domain may be termed an engager in that it engages to the target molecule it binds. Accordingly, in some cases a target for a binder may refer to the protein or the polypeptide or the biomolecule to which the binding domain binds. A target may be located on a different cell from the cell on which a binding domain may be located. In some embodiments, the cell on which the target (e.g., the protein or the biomolecule to which the binder binds), may be referred to as a target cell. In some cases, a binder may not be located on a cell, e.g., a cellular, or may be referred to in such cases as a soluble binder. A binder may be an antibody, or any fragments thereof, an scFV, a sdAb, a VHH.

Often, “antibody” as used herein may refer to an antibody, an scFv, a VHH, single domain antibody (sdAb), or a protein or polypeptide that comprises an inactive antigen binding domain; wherein the antigen binding capability is designed to be blocked or inactive e.g., by binding a cleavable antigen domain binding polypeptide, until an active step is performed to convert the pro-antibody to its active form. In some embodiments, the active step involves a protease cleavage of the entity that block the antigen binding domain.

In many instances, the term “affinity” may refer to a quality of ability of one molecule (e.g., a protein molecule) to bind to another molecule or a ligand that binds with chemical specificity. Generally, a molecule a having higher affinity to bind another molecule b than to a third molecule c, would bind more strongly to b and to c. Chemical specificity is the ability of a protein's binding site to bind specific ligands. The fewer ligands a protein can bind, the greater its specificity. Specificity describes the strength of binding between a given protein and ligand. This relationship can be described by a first scFv specific to a cell surface component on a dissociation constant (KD), which characterizes the balance between bound and unbound states for the protein-ligand system.

In many instances, the term “antigen-presenting cell” or “antigen-presenting cells” or its abbreviation “APC” or “APCs” may refer to a cell or cells capable of endocytosis adsorption, processing and presenting of an antigen. The term includes professional antigen presenting cells, for example, B lymphocytes, monocytes, dendritic cells (DCs) and Langerhans cells, as well as other antigen presenting cells such as keratinocytes, endothelial cells, glial cells, fibroblasts and oligodendrocytes. The term “antigen presenting” may mean the display of antigen as peptide fragments bound to MHC molecules, on the cell surface. Many different kinds of cells may function as APCs including, for example, monocytes or macrophages, B cells, follicular dendritic cells and dendritic cells. APCs can also cross-present peptide antigens by processing exogenous antigens and presenting the processed antigens on class I MHC molecules. Antigens that give rise to proteins that are recognized in association with class I MHC molecules may generally be proteins that are produced within the cells, and these antigens are processed and associate with class I MHC molecules.

An “epitope” may refer to a portion of an antigen or other macromolecule capable of forming a binding interaction with the variable region binding pocket of an antibody or TCR. The term includes any protein determinant capable of specific binding to an antibody, antibody peptide, and/or antibody-like molecule (including but not limited to a T cell receptor) as defined herein. Epitopic determinants typically consist of chemically active surface groups of molecules such as amino acids or sugar side chains and generally have specific three-dimensional structural characteristics as well as specific charge characteristics.

In many instances, the term “antigen” may be any organic or inorganic molecule capable of stimulating an immune response. The term “antigen” as used herein extends to any molecule such as, but not limited, to a peptide, polypeptide, protein, nucleic acid molecule, carbohydrate molecule, organic or inorganic molecule capable of stimulating an immune response.

In many instances, “antibody” or “antibody moiety” may include but is not limited to any polypeptide chain-containing molecular structure that recognizes an epitope. Antibodies may utilize in the present invention may be polyclonal antibodies, although monoclonal antibodies are preferred because they may be reproduced by cell culture or recombinantly and can be modified to reduce their antigenicity. The term includes IgG (including IgG1, IgG2, IgG3, and IgG4), IgA (including IgA1 and IgA2), IgD, IgE, IgM, and IgY, and is meant to include whole antibodies, including single-chain whole antibodies, and antigen-binding (Fab) fragments thereof Δntigen-binding antibody fragments include, but are not limited to, Fab, Fab′ and F(ab′)2, Fd (consisting of V_{H} and CH1), single-chain variable fragment (scFv), single-chain antibodies, disulfide-linked variable fragment (dsFv) and fragments comprising either a V_{L} or V_{H} domain. The antibodies can be from any animal origin. Antigen-binding antibody fragments, including single-chain antibodies, can comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex. A person of skill in the art will recognize that a variety of immunoaffinity techniques are suitable to enrich soluble proteins, such as soluble HLA-peptide complexes or membrane bound HLA-associated polypeptides, e.g., which have been proteolytically cleaved from the membrane. These include techniques in which, for example, (1) one or more antibodies capable of specifically binding to the soluble protein are immobilized to a fixed or mobile substrate (e.g., plastic wells or resin, latex or paramagnetic beads), and (2) a solution containing the soluble protein from a biological sample is passed over the antibody coated substrate, allowing the soluble protein to bind to the antibodies. The substrate with the antibody and bound soluble protein is separated from the solution, and optionally the antibody and soluble protein are disassociated, for example by varying the pH and/or the ionic strength and/or ionic composition of the solution bathing the antibodies. Alternatively, immunoprecipitation techniques in which the antibody and soluble protein are combined and allowed to form macromolecular aggregates can be used. The macromolecular aggregates can be separated from the solution by size exclusion techniques or by centrifugation.

The adaptive immune system reacts to molecular structures, referred to as antigens, of the intruding organism. Unlike the innate immune system, the adaptive immune system is highly specific to a pathogen. Adaptive immunity can also provide long-lasting protection; for example, someone who recovers from measles is now protected against measles for their lifetime. There are two types of adaptive immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in T cells, of the body. Cytotoxic T cells recognize these antigens and kill the cells that harbor the antigens.

In many instances, the term “major histocompatibility complex (MHC)”, “MHC molecules”, or “MHC proteins” may refer to proteins capable of binding antigenic peptides resulting from the proteolytic cleavage of protein antigens inside phagocytes or antigen presenting cells and for the purpose of presentation to and activation of T lymphocytes. Such antigenic peptides may represent T cell epitopes. The human MHC is also called the HLA complex. Thus, the term “human leukocyte antigen (HLA) system”, “HLA molecules” or “HLA proteins” may refer to a gene complex encoding the MHC proteins in humans. The term MHC may be referred as the “H-2” complex in murine species. Those of ordinary skill in the art would recognize that the terms “major histocompatibility complex (MHC)”, “MHC molecules”, “MHC proteins” and “human leukocyte antigen (HLA) system”, “HLA molecules”, “HLA proteins” are used interchangeably herein.

HLA proteins are typically classified into two types, referred to as HLA class I and HLA class II. The structures of the proteins of the two HLA classes are very similar; however, they can have different functions. Class I HLA proteins are present on the surface of almost all cells of the body, including most tumor cells. Class I HLA proteins are loaded with antigens that usually originate from endogenous proteins or from pathogens present inside cells and are then presented to naïve or cytotoxic T-lymphocytes (CTLs). HLA class II proteins are present on antigen presenting cells (APCs), including but not limited to dendritic cells, B cells, and monocytes or macrophages. They mainly present peptides, which are processed from external antigen sources, e.g., outside of the cells, to helper T cells. Most of the peptides bound by the HLA class I proteins originate from cytoplasmic proteins produced in the healthy host cells of an organism itself, and do not normally stimulate an immune reaction.

In HLA class II system, phagocytes such as monocytes or macrophages and immature dendritic cells take up entities by phagocytosis into phagosomes—though B cells exhibit the more general endocytosis into endosomes—which fuse with lysosomes whose acidic enzymes cleave the uptaken protein into many different peptides. Autophagy is a source of HLA class II peptides. Via physicochemical dynamics in molecular interaction with the HLA class II variants borne by the host, encoded in the host's genome, a particular peptide exhibits immunodominance and loads onto HLA class II molecules. These are trafficked to and externalized on the cell surface. The most studied subclass II HLA genes are: HLA-DPA1, HLA-DPB1, HLA-DQA1, HLA-DQB1, HLA-DRA, and HLA-DRB1.

Presentation of peptides by HLA class II molecules to CD4+ helper T cells is required for immune responses to foreign antigens. Once activated, CD4+ T cells promote B cell differentiation and antibody production, as well as CD8+ T cell (CTL) responses. CD4+ T cells also secrete cytokines and chemokines that activate and induce differentiation of other immune cells. HLA class II molecules are heterodimers of α- and β-chains that interact to form a peptide-binding groove that is more open than class I peptide-binding grooves. Peptides bound to HLA class II molecules are believed to have a 9-amino acid binding core with flanking residues on either N- or C-terminal side that overhang from the groove. These peptides are usually 12-16 amino acids in length and often contain 3-4 anchor residues at positions P1, P4, P6/7 and P9 of the binding register (Rossjohn et al., 2015).

HLA alleles are expressed in codominant fashion, meaning that the alleles (variants) inherited from both parents are expressed equally. For example, each person carries 2 alleles of each of the 3 class I genes, (HLA-A, HLA-B and HLA-C) and so can express six different types of class II HLA. In the class II HLA locus, each person inherits a pair of HLA-DP genes (DPA1 and DPB1, which encode α and β chains), HLA-DQ (DQA1 and DQB1, for a and 1 chains), one gene HLA-DRα (DRA1), and one or more genes HLA-DRO (DRB1 and DRB3, -4 or -5). HLA-DRB1, for example, has more than nearly 400 known alleles. That means that one heterozygous individual can inherit six or eight functioning class II HLA alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the class I HLA alleles are HLA-A*02:01, HLA-B*14:02, HLA-A*23:01, HLA-E*01:01 (non-classical). In some embodiments, class II HLA alleles are HLA-DRB*01:01, HLA-DRB*01:02, HLA-DRB*11:01, HLA-DRB*15:01, and HLA-DRB*07:01.

Subject specific HLA alleles or HLA genotype of a subject can be determined by any method known in the art. In exemplary embodiments, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

“Amino acid” as used herein may be intended to include both natural and synthetic amino acids, and both D and L amino acids. A synthetic amino acid also encompasses chemically modified amino acids, including, but not limited to salts, and amino acid derivatives such as amides. Amino acids present within the polypeptides of the present invention can be modified to form modified amino acids by methylation, amidation, acetylation or substitution with other chemical groups which can change the circulating half-life without adversely affecting their biological activity.

In some instances, “operably linked,” “linked,” “fused” or “connected” may be used for interchangeably describing two structural units or subunits being structurally connected with each other. The connection may be direct, that is without any other components between the two, or it may be indirect, wherein one or more linkers as described elsewhere in the specification may be connecting the two units in the description.

In several occurrences throughout the document, the terms “peptide”, “polypeptide” and “protein” may be used herein interchangeably to describe a series of at least two amino acids covalently linked by peptide bonds or modified peptide bonds such as isosteres. No limitation is placed on the maximum number of amino acids which may comprise a peptide or protein. The terms “oligomer” and “oligopeptide” are also intended to mean a peptide as described herein. Furthermore, the term polypeptide extends to fragments, analogues and derivatives of a peptide, wherein said fragment, analogue or derivative retains the same biological functional activity as the peptide from which the fragment, derivative or analogue is derived.

A polypeptide as used herein may be a “protein”, including but not limited to a glycoprotein, a lipoprotein, a cellular protein or a membrane protein. A polypeptide may comprise one or more subunits of a protein. A polypeptide may be encoded by a recombinant nucleic acid. In some embodiments, In some embodiments, a polypeptide described herein comprises one or more structurally distinct domains. In some embodiments, each domain of a polypeptide described herein may have a distinct function. Usually, a domain is a structural portion of a protein or polypeptide with a defined function. A moiety is a portion of polypeptide, a protein or a nucleic acid, having a specific structure or perform a specific function. For example, a signaling moiety is a specific unit within the larger structure of the polypeptide or protein or a recombinant nucleic acid, which (or the protein portion encoded by it in case of a nucleic acid) engages in a signal transduction process, for example a phosphorylation. In some examples, two or more domains may be required to accomplish a single function. In some instances, the two or more domains required to accomplish a single function may reside on two or more different polypeptides, and the function is accomplished only when a given three-dimensional structure is achieved, for example, by oligomerization and proper orientation of the two or more different polypeptides. For example, an MHC-peptide complex engager described herein may comprise a TRAV domain and a TRBV domain, each residing on a different polypeptide and dimerization of the two polypeptides facilitate attaining the configuration required for binding to an MHC-peptide complex.

In several occurrences throughout the document, p-MHC may refer to a structural unit of a combination of an antigenic peptide and its cognate MHC. An MHC molecule is encoded by an HLA gene and may be an MHC class I allele and an MHC class II allele. In general, a peptide associated with an MHC class I molecule is shorter in length (9-11 amino acids) than that associated with an MHC class II molecule. A peptide presented in a MHC class I molecule may generally be presented to and activate a CD8+ T cell. A peptide presented in a MHC class II molecule may generally be presented to and activate a CD4+ T cell. An MHC class I peptide may refer to a peptide that is presented in association with an MHC class I molecule, and an MHC class I associated peptide may interchangeably be called a CD8 peptide or semantic variations thereof Δn MHC class II peptide may likewise refer to a peptide that is presented in association with an MHC class II molecule, and an MHC class II associated peptide may interchangeably be called a CD4 peptide or semantic variations thereof.

As used herein, the term “recombinant nucleic acid molecule” may refer to a recombinant DNA molecule or a recombinant RNA molecule. A recombinant nucleic acid molecule may be any nucleic acid molecule containing joined nucleic acid molecules from different original sources and not naturally attached together. A recombinant nucleic acid may be synthesized in the laboratory. A recombinant nucleic acid can be prepared by using recombinant DNA technology by using enzymatic modification of DNA, such as enzymatic restriction digestion, ligation, and DNA cloning. A recombinant nucleic acid as used herein can be DNA, or RNA. A recombinant DNA may be transcribed in vitro, to generate a messenger RNA (mRNA), the recombinant mRNA may be isolated, purified and used to transfect a cell. A recombinant nucleic acid may encode a protein or a polypeptide. A recombinant nucleic acid, under suitable conditions, can be incorporated into a living cell, and can be expressed inside the living cell. As used herein, “expression” of a nucleic acid usually refers to transcription and/or translation of the nucleic acid. The product of a nucleic acid expression is usually a protein but can also be an mRNA. Detection of an mRNA encoded by a recombinant nucleic acid in a cell that has incorporated the recombinant nucleic acid, is considered positive proof that the nucleic acid is “expressed” in the cell.

The process of inserting or incorporating a nucleic acid into a cell can be via transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the forced introduction of small molecules such as DNA, RNA, or antibodies into eukaryotic cells. Just to make life confusing, ‘transfection’ also refers to the introduction of bacteriophage into bacterial cells. ‘Transduction’ is mostly used to describe the introduction of recombinant viral vector particles into target cells, while ‘infection’ refers to natural infections of humans or animals with wild-type viruses.

As used herein, the term “vector” may mean any genetic construct, such as a plasmid, phage, transposon, cosmid, chromosome, virus, virion, etc., which is capable transferring nucleic acids between cells. Vectors may be capable of one or more of replication, expression, recombination, insertion or integration, but need not possess each of these capabilities. A plasmid is a species of the genus encompassed by the term “vector.” A vector typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA molecules which, in their vector form are not bound to the chromosome, and typically comprise entities for stable or transient expression or the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the cell. A vector can be a DNA or RNA vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

The terms “spacer” or “linker” as used in reference to a fusion protein may refer to a peptide that joins the proteins comprising a fusion protein. In some embodiments, the constituent amino acids of a spacer can be selected to influence some property of the molecule such as the folding, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. The linker is used to separate two antigenic peptides by a distance sufficient to ensure that, in some embodiments, each antigenic peptide properly folds. Exemplary peptide linker sequences adopt a flexible extended conformation and do not exhibit a propensity for developing an ordered secondary structure. Typical amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, also can be used in the linker sequence. In some cases, linkers may be designed with combinations of Gly (G) and Ser (S). For example, G4S would refer to a linker that may comprise a sequence of GGGGS. For example, in some cases, the linker sequence is GGGGS; or for example, in some cases the linker sequence is a concatemer of GGGGS. Linkers used herein are not limited to those described above. Other near neutral amino acids, such as Thr and Ala, also can be used in the linker sequence. Linkers used herein may include linkers that have been available in the public domain, e.g., published sequences or commercially available. Linkers used herein may include linkers that are designed in-house.

In some embodiments, the peptide linkers have more than one functional properties, such as the ones described herein. For example, the peptide linker links two or more functional domains, such as binding domains. Additionally, the peptide linker may be a specific signal inducer when the linker contacts an extracellular portion of a cell, such as a receptor or a ligand binding protein.

The term “immunopurification (IP)” (or immunoaffinity purification or immunoprecipitation) may refer to a process well known in the art and is widely used for the isolation of a desired antigen from a sample. In general, the process involves contacting a sample containing a desired antigen with an affinity matrix comprising an antibody to the antigen covalently attached to a solid phase. The antigen in the sample becomes bound to the affinity matrix through an immunochemical bond. The affinity matrix is then washed to remove any unbound species. The antigen is removed from the affinity matrix by altering the chemical composition of a solution in contact with the affinity matrix. The immunopurification can be conducted on a column containing the affinity matrix, in which case the solution is an eluent. Alternatively, the immunopurification can be in a batch process, in which case the affinity matrix is maintained as a suspension in the solution. An important step in the process is the removal of antigen from the matrix. This is commonly achieved by increasing the ionic strength of the solution in contact with the affinity matrix, for example, by the addition of an inorganic salt. An alteration of pH can also be effective to dissociate the immunochemical bond between antigen and the affinity matrix.

As used herein, the terms “determining”, “assessing”, “assaying”, “measuring”, “detecting” and their grammatical equivalents refer to both quantitative and qualitative determinations, and as such, the term “determining” may be used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte may be used.

A “fragment” may be a portion of a protein or nucleic acid that is substantially identical to a reference protein or nucleic acid. In some embodiments, the portion retains at least 50%, 75%, or 80%, or 90%, 95%, or even 99% of the biological activity of the reference protein or nucleic acid described herein.

The terms “isolated,” “purified”, “biologically pure” and their grammatical equivalents refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

A “cancer” may refer to any disease that is caused by or results in inappropriately high levels of cell division, inappropriately low levels of apoptosis, or both. Glioblastoma is one non-limiting example of a neoplasia or cancer. The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell.

As used herein an “MHC-peptide complex engager” may refer to a one or more domains that bind to an MHC-peptide complex. An MHC-peptide complex engager described herein may comprise a TRAV domain and a TRBV domain, each residing on a different polypeptide and dimerization of the two polypeptides facilitate attaining the configuration required for binding to an MHC-peptide complex.

As used herein a “T cell engager” may refer to a domain that binds to a molecule of a T cell, such as a protein expressed by a T cell. A T cell engager described herein may comprise a single chain variable fragment (scFv) capable of binding to a target antigen/molecule of a T cell, such as a protein expressed by a T cell. A T cell engager described herein may comprise a VHH capable of binding to a molecule of a T cell, such as a protein expressed by a T cell. In one instance, where the molecule of the T cell or the protein expressed by the T cell is a receptor, the T cell engager may be a ligand capable of binding to the receptor.

As used herein, the term “pharmaceutically acceptable” may refer to approved or approvable by a regulatory agency of the Federal or a state government or listed in the U.S. Pharmacopeia or other generally recognized pharmacopeia for use in animals, including humans. A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent. A “pharmaceutically acceptable salt” of pooled disease specific antigens as recited herein can be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluene sulfonic, methane sulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled disease specific antigens provided herein, including those listed by Remington's Pharmaceutical Sciences, 17th ed., Mack Publishing Company, Easton, PA, p. 1418 (1985).

Nucleic acid molecules useful in the methods of the disclosure may include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having substantial identity to an endogenous sequence are typically capable of hybridizing with at least one strand of a double-stranded nucleic acid molecule. “Hybridize” refers to when nucleic acid molecules pair to form a double-stranded molecule between complementary polynucleotide sequences, or portions thereof, under various conditions of stringency. For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions can ordinarily include temperatures of at least about 30 °C, at least about 37 °C, or at least about 42 °C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an exemplary embodiment, hybridization can occur at 30 °C in 750 mM NaCl, 75 mM trisodium citrate, and 1% SDS. In another exemplary embodiment, hybridization can occur at 37 °C in 500 mM NaCl, 50 mM trisodium citrate, 1% SDS, 35% formamide, and 100 μg/ml denatured salmon sperm DNA (ssDNA). In another exemplary embodiment, hybridization can occur at 42 °C in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25 °C, of at least about 42 °C, or at least about 68 °C. In exemplary embodiments, wash steps can occur at 25 °C in 30 mM NaCl, 3 mM trisodium citrate, and 0.1% SDS. In other exemplary embodiments, wash steps can occur at 42 °C in 15 mM NaCl, 1.5 mM trisodium citrate, and 0.1% SDS. In another exemplary embodiment, wash steps can occur at 68 °C in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Substantially identical” may be used in reference to comparison between sequences of two or more polypeptide or nucleic acid molecules, for example, the sequence of one polypeptide may be described as exhibiting at least 50% identity to a reference amino acid sequence. A reference sequence may be a sequence disclosed in this specification or referred to as disclosed in another publicly available source. Such a sequence can be at least 60%, 80% or 85%, 90%, 95%, 96%, 97%, 98%, or even 99% or more identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. A “reference” is a standard of comparison.

In several occurrences, the term “scaffold” may refer to a molecular platform structure which may be further tweaked to render it suitable for a specific purpose, In the context described herein, a scaffold may refer to a multispecific T cell engager, that may comprise at least one arm that binds to a cell that presents an epitope of interest on its cell surface and at least a second arm that can bind to a T cell that can engage with the cell presenting the epitope and can do one or more of the following: destroy the cell, activate an immune response specific to the epitope, deactivate the cell.

The term “subject” or “patient” may refer to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The alpha (a) and beta (b) chains of abTCR's are generally regarded as each having two “domains,” namely variable and constant domains. The variable domain may consist of a concatenation of variable region, and joining region, and is usually at the terminal portion of the TCR chain. TCR alpha variable domain may therefore refer to the concatenation of TRAV, and TRAJ regions, and the term TCR alpha constant domain refers to the extracellular TRAC region, or to a C-terminal truncated TRAC sequence. TCR beta variable domain may likewise refer to the concatenation of TRBV and TRBD/TRBJ regions, and TCR beta constant domain may refer to the extracellular TRBC region, or to a C-terminal truncated TRBC sequence.

The terms “treat,” “treated,” “treating,” “treatment,” “to treat” or “alleviating” or “to alleviate” refer to therapeutic measures that cure, slow down, lessen symptoms of, and/or halt progression of a diagnosed pathologic condition or disorder. Thus, those in need of treatment may include those already with the disorder. In some cases, treating may refer to reducing or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” includes the concepts of “alleviating”, which refers to lessening the frequency of occurrence or recurrence, or the severity, of any symptoms or other ill effects related to the disease and/or the side effects associated with therapy. The term “treating” may also encompass the concept of “managing” which refers to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The terms “prevent” or “prevention” refer to prophylactic or preventative measures that slow down the development of a targeted pathologic condition or disorder. Thus, those in need of prevention include those prone to have the disorder or those in whom the disorder is to be prevented.

The term “therapeutic effect” refers to some extent of relief of one or more of the symptoms of a disorder (e.g., a neoplasia, tumor, or infection by an infectious agent or an autoimmune disease) or its associated pathology. “Therapeutically effective amount” as used herein may refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

Whereas a cancer cell or a tumor cell may be repeatedly referred here as the target cell, the concepts described here can be suitable for any type of a target cell, such as an infected cell, or a specific disease cell type that needs to be eliminated by the immune cells, as long as the binding domain for a cell surface component of a cancer cell is suitably replaced by a binding domain for a cell surface component specific for the target cell.

The T cell receptor (TCR) of a T cell is able to interact with immunogenic peptides (epitopes) bound to major histocompatibility complex (NIHC) molecules and presented on the surface of target cells. Specific binding of the TCR triggers a signal cascade inside the T cell leading to proliferation and differentiation into a maturated effector T cell. To be able to target a vast variety of antigens, the T cell receptors need to have a great diversity.

This diversity is obtained by genetic rearrangement of different discontinuous segments of genes which code for the different structural regions of TCRs. TCRs are composed of one α-chain and one β-chain or of one γ-chain and one δ-chain. The TCR α/β chains are composed of an N-terminal highly polymorphic variable region involved in antigen recognition and an invariant constant region. On the genetic level, these chains are separated into several regions, a variable (V) region, a diversity (D) region (only β- and δ-chain), a joining (J) region and a constant (C) region. The human β-chain genes contain over 60 variable (V), 2 diversity (D), over 10 joining (J) segments, and 2 constant region segments (C). The human α-chain genes contain over 50 V segments, and over 60 J segments but no D segments, as well as one C segment. The murine β-chain genes contain over 30 variable (V), 2 diversity (D), over 10 joining (J) segments, and 2 constant region segments (C). The murine α-chain genes contain almost 100 V segments, 60 J segments, no D segments, but one C segment. During the differentiation of T cells, specific T cell receptor genes are created by rearranging one V, one D (only β- and δ-chain), one J and one C region gene. The diversity of the TCRs is further amplified by imprecise V-(D)-J rearrangement wherein random nucleotides are introduced and/or deleted at the recombination sites. Since the rearrangement of the TCR gene loci occurs in the genome during maturation of T cells, each mature T cell only expresses one specific α/β TCR or γ/δ TCR.

MHC and antigen binding is mediated by the complementary determining regions 1, 2 and 3 (CDR1, CDR2, CDR3) of the TCR. The CDR3 of the β-chain which is most critical for antigen recognition and binding is encoded by the V-D-J junction of the rearranged TCR β-chain gene.

The TCR is a part of a complex signaling machinery, which includes the heterodimeric complex of the TCR α- and β-chains, the co-receptor CD4 or CD8 and the CD3 signal transduction module. While the CD3 chains transfer the activation signal inside the cell, the TCR α/β heterodimer is solely responsible for antigen recognition. Thus, the transfer of the TCR α/β chains offers the opportunity to redirect T cells towards any antigen of interest.

Some natural TCRs may have weak affinities and low stabilities for peptide: MHC complexes, limiting their potential to reach efficacious therapy as soluble drugs. It has been observed that although the buried surface area created at the TCR: pHLA protein-protein interface is large (around 2000 angstrom² on average), the naturally measured binding affinity is relatively weak (KD ~0.1-1000 μM) compared with other Ig-like proteins (Bridgeman J S, Sewell A K, Miles J J, Price D & Cole D K (2012) Structural and biophysical determinants of alphabeta T-cell antigen recognition. Immunology 135, 9-18). TCRs selective for tumor-associated pHLAs tend to bind towards the weaker end of this range, likely representing the thymic deletion of TCRs binding with strong affinity to self-derived tumor peptides, adding to the challenge of TCR selection and their utility as therapeutics in this disease area. To overcome these limitations and make full use of the TCR, several genetic and protein engineering solutions have been applied to TCRs to enhance both their stability and affinity. Antigen-specific CD8+ T cell-mediated immune responses depend on the appropriate recognition of the α/β T-cell receptor (TCR) against peptide-major histocompatibility class I (MHC I) molecule complexes. The binding site of the TCR includes three complementarity-determining regions (CDRs) for each chain in which CDR3 is the most diverse and important CDR in antigen recognition. Methods to improve TCR affinity include the introduction of amino acid sequence variations into the TCR complementarity-determining regions (CDRs).

TCR mimic antibodies for peptide HLA require either extensive panning from large (>10¹¹) phage/yeast display VH/VL libraries and/or animal (i.e., camelid, mouse) immunization and screening. Unlike TCRs, antibodies do not start in a “pHLA-facing” pose, and affinity maturation has to occur, starting from scratch. Personalized medicine is time and cost intensive. There is no option ‘off-the-shelf’ approach.

In one aspect, soluble TCRs may be contemplated herein, designed, prepared and tested. Soluble TCRs are TCRs that comprise an alpha chain and a beta chain corresponding to natural TCRs but are not membrane anchored. The soluble TCRs may be engineered as nucleic acid constructs and may be incorporated into a cell of interest, and that are then translated and released extracellularly. Soluble TCRs can bind to one, two or three different targets, wherein the targets are antigens, antigens in complex with MHC or various combinations thereof as designed, where the targets are present in one or more cells in close proximity. For example, a soluble TCR designed to bind to two different targets may have a first target that binds to a cytotoxic T cell, and a second target that binds to a cancer antigen on a cancer cell, thereby bonding the cytotoxic cell to the cancer cell. For example in one embodiment, when a soluble TCR is designed to bind to a target on a desired cell, the binding may generate signal transduction in the target cell to activate the target cell.

Engineering T cells are yet another option investigated with great interest. T cells engineered to express TCRs that have been designed for higher specificity to the target epitope. However, for the purpose of this work, engineering T cells safely and effectively is not straight-forward. A customable but off-the-shelf platform is hereby sought that is easier to generate and manufacture and effective as a therapeutic. Developing technologies to stabilize and enhance the activity of a soluble TCR outside of the cell could unlock new therapeutic modalities and expand access for patients.

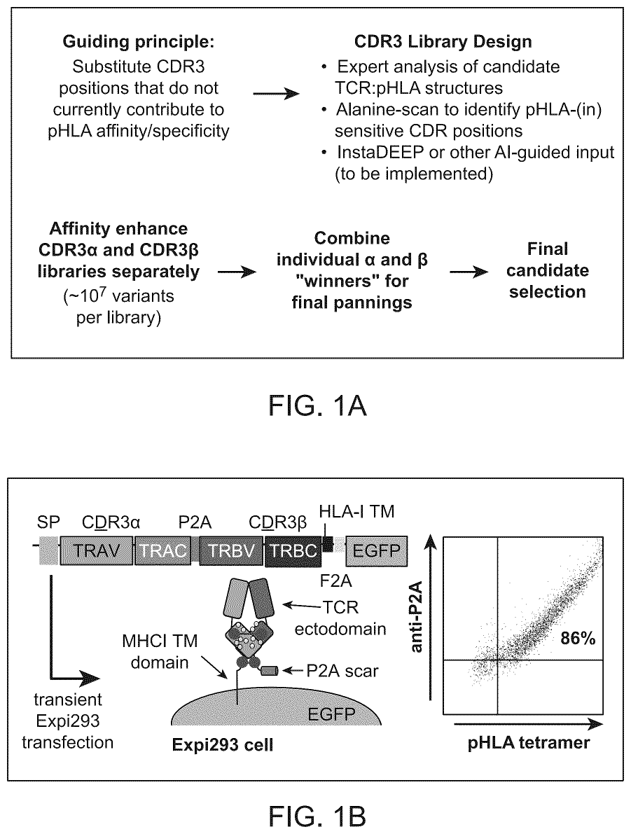

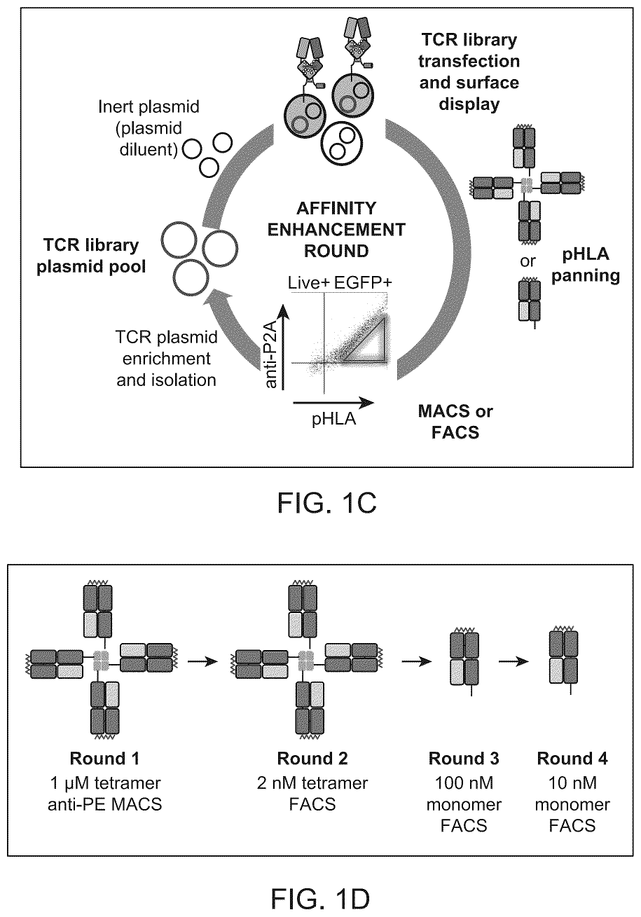

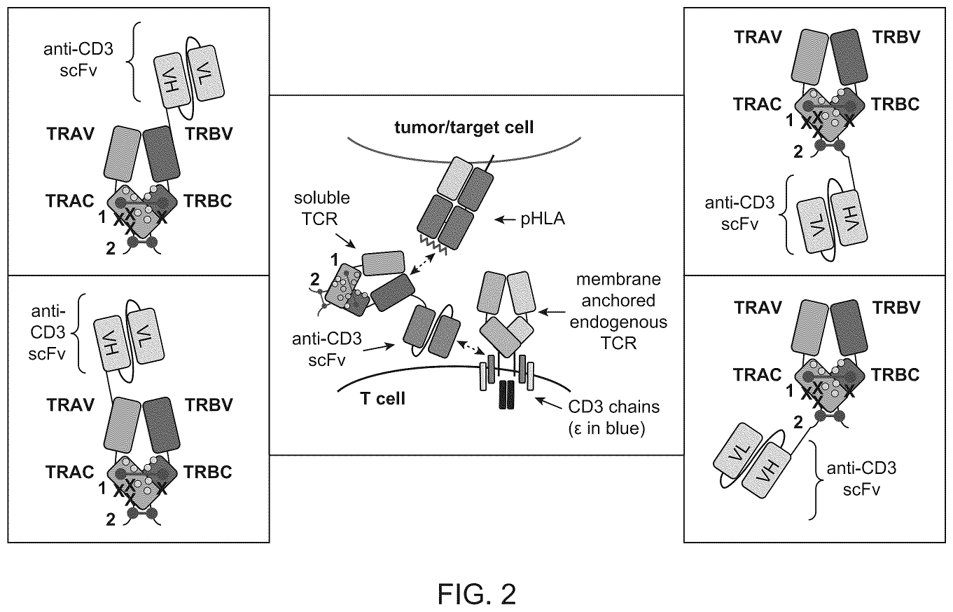

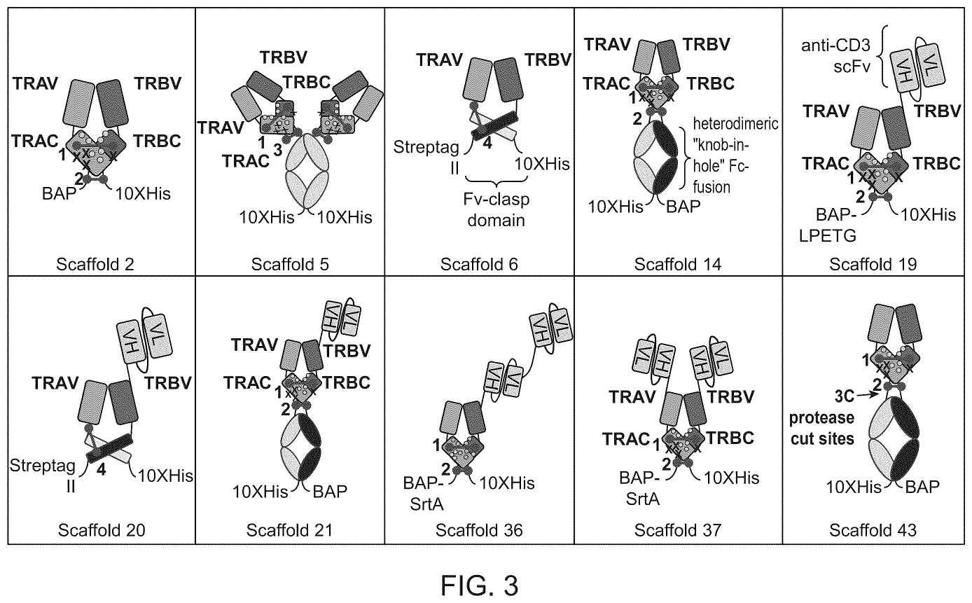

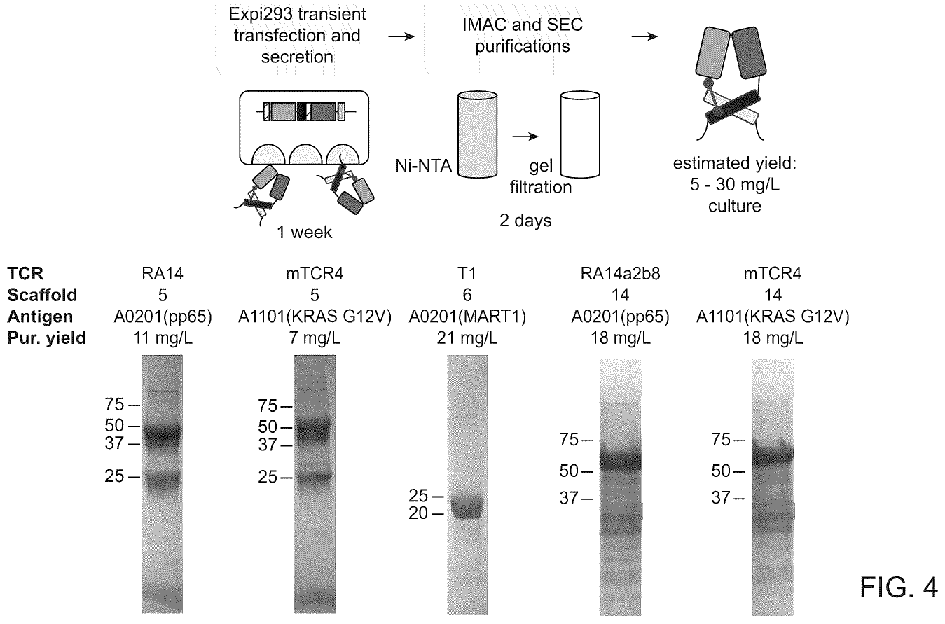

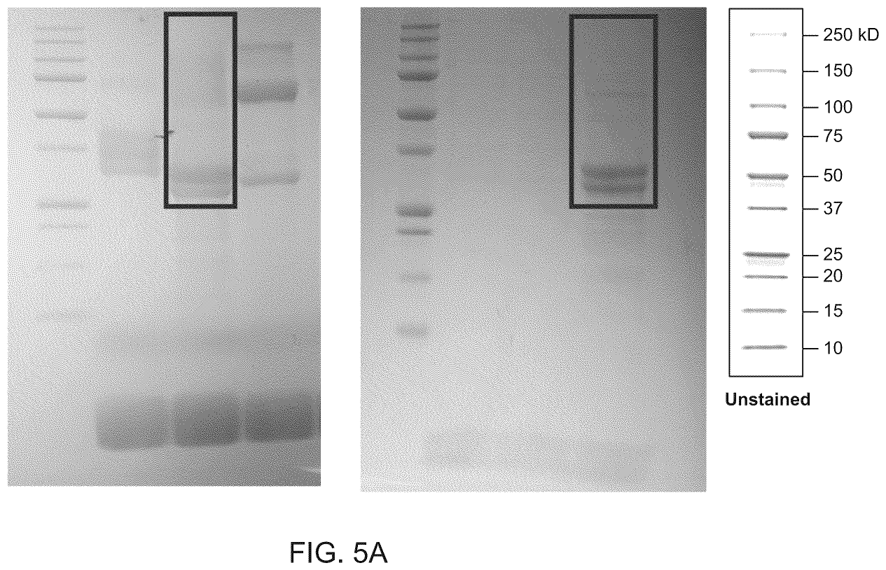

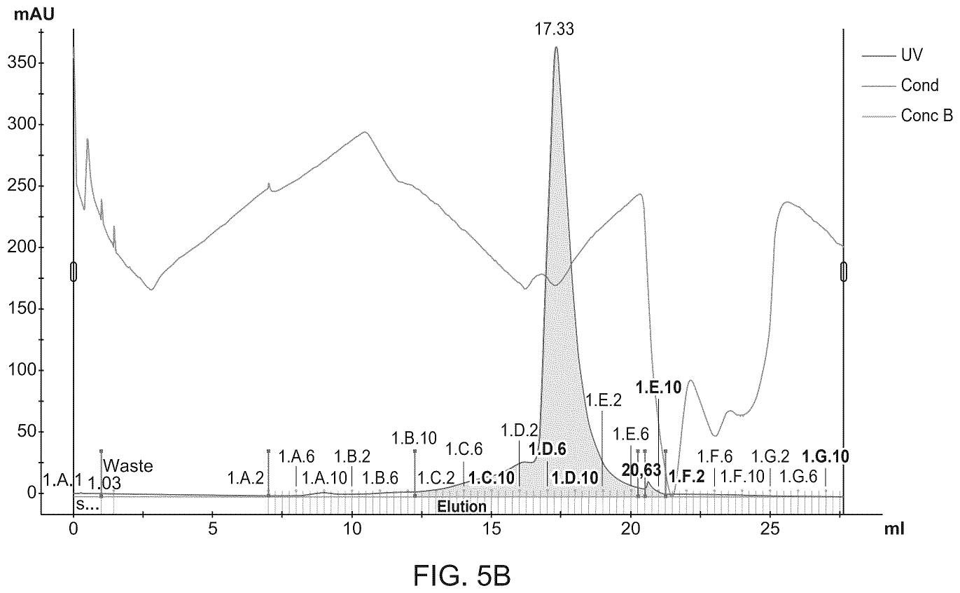

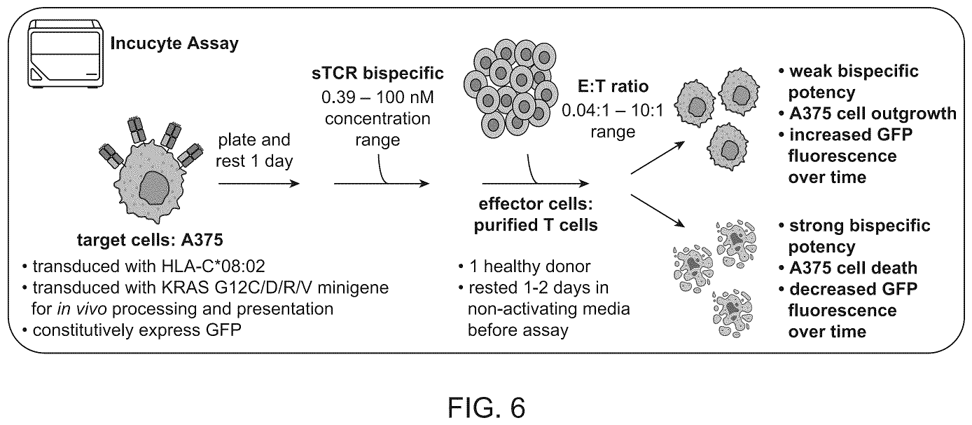

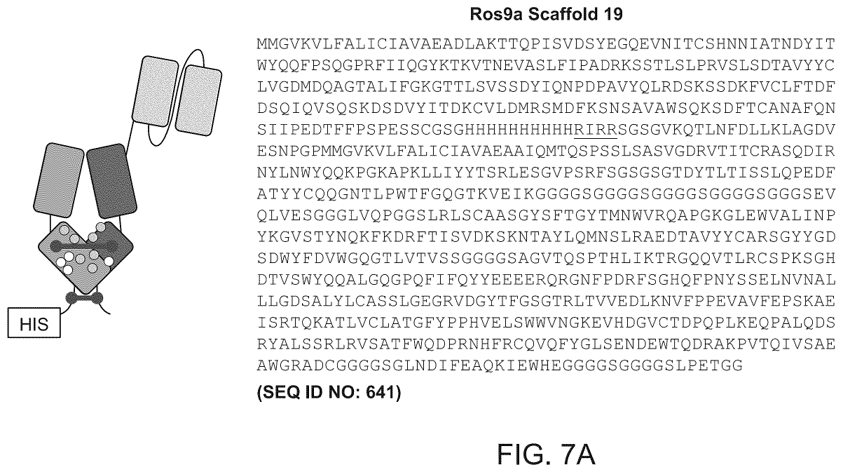

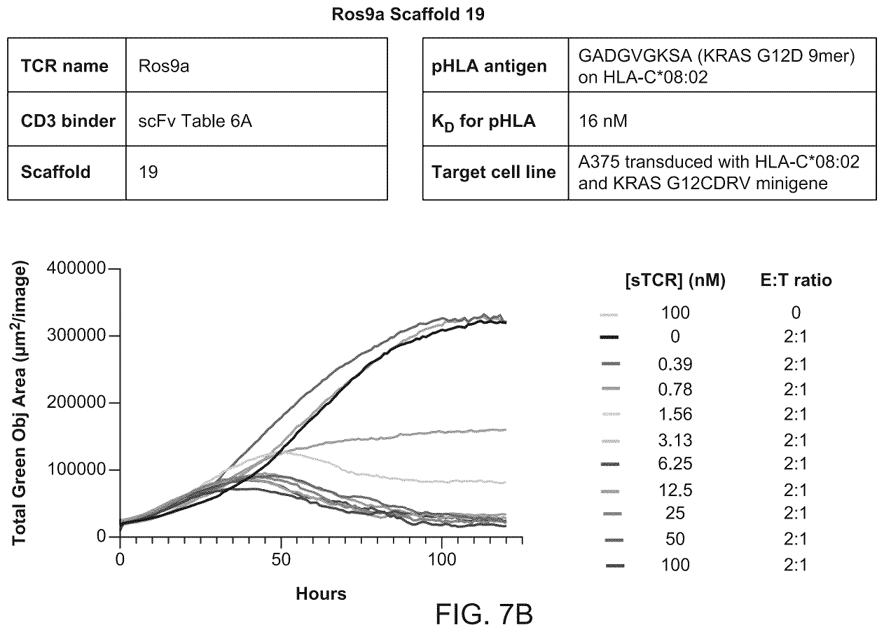

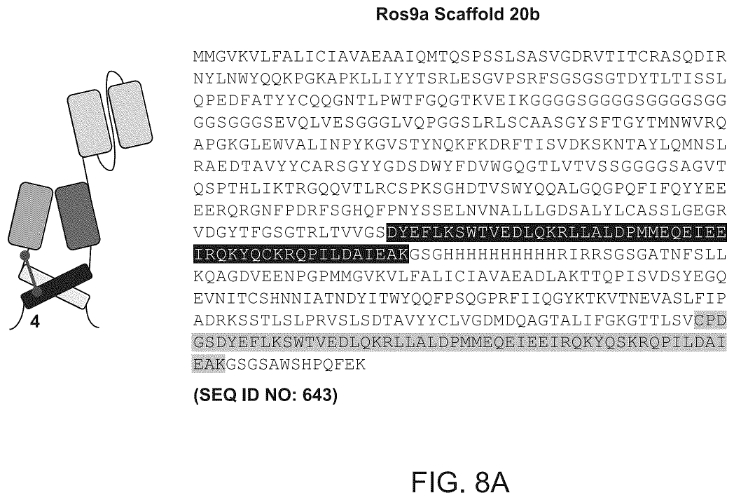

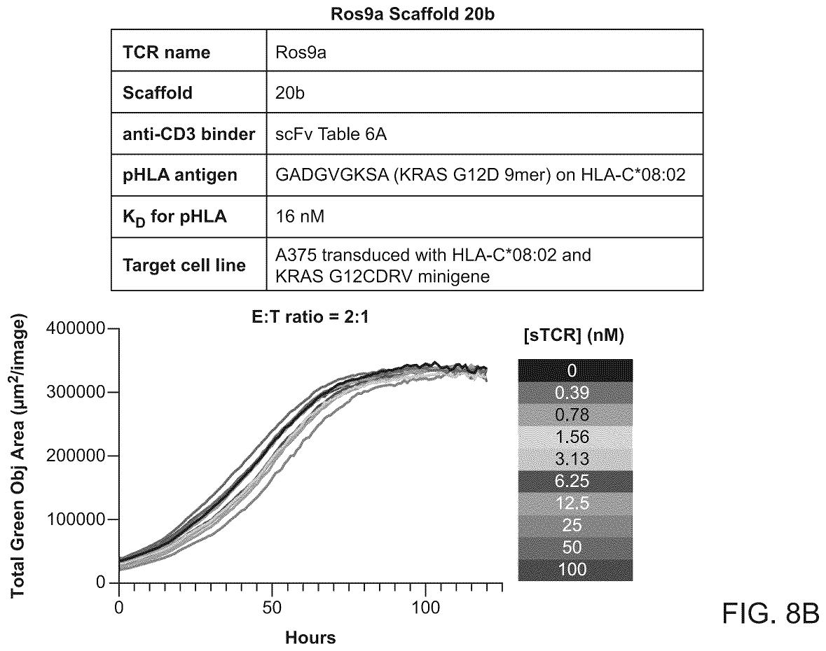

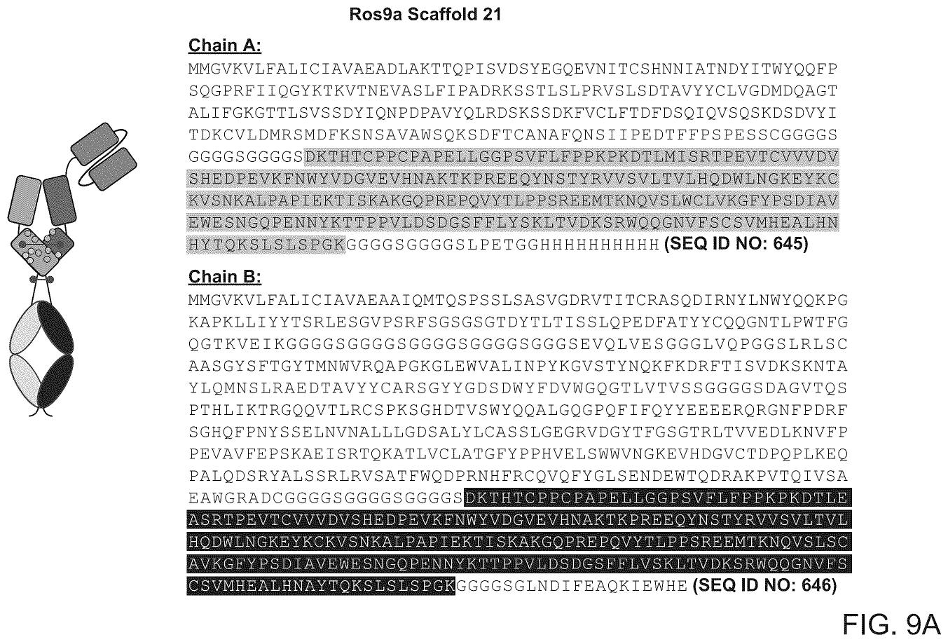

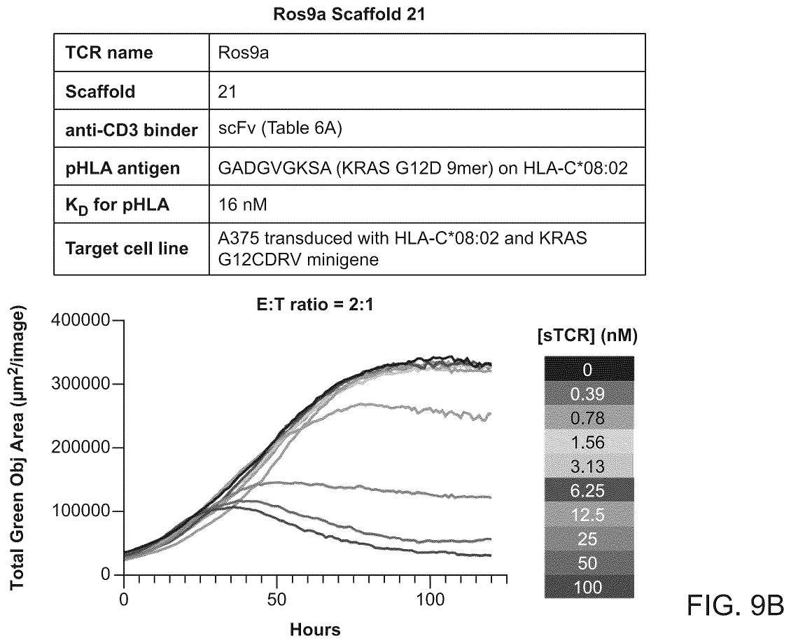

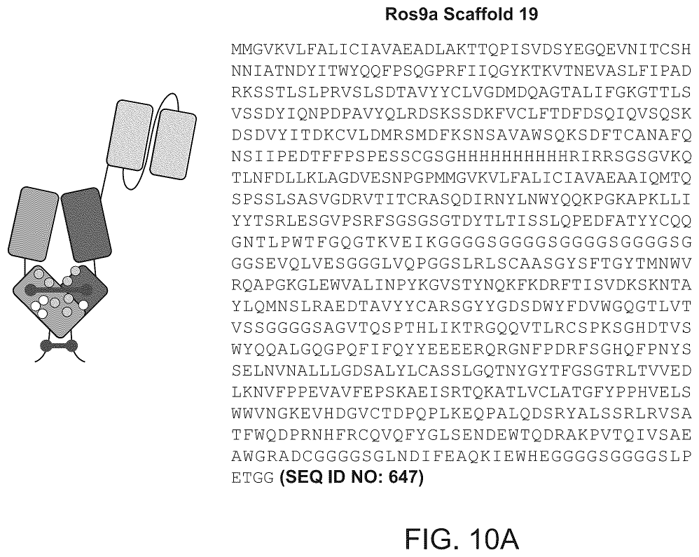

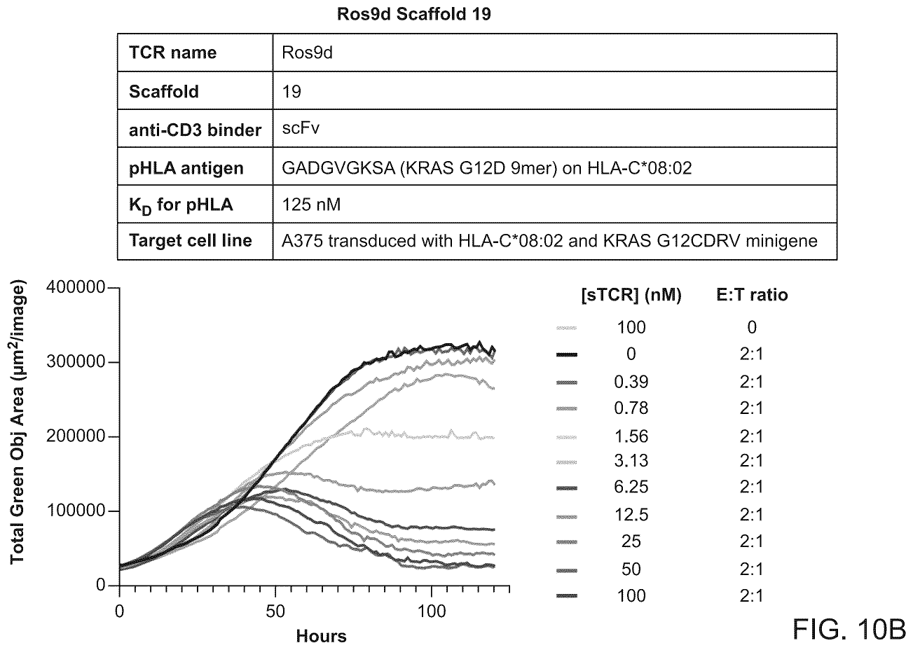

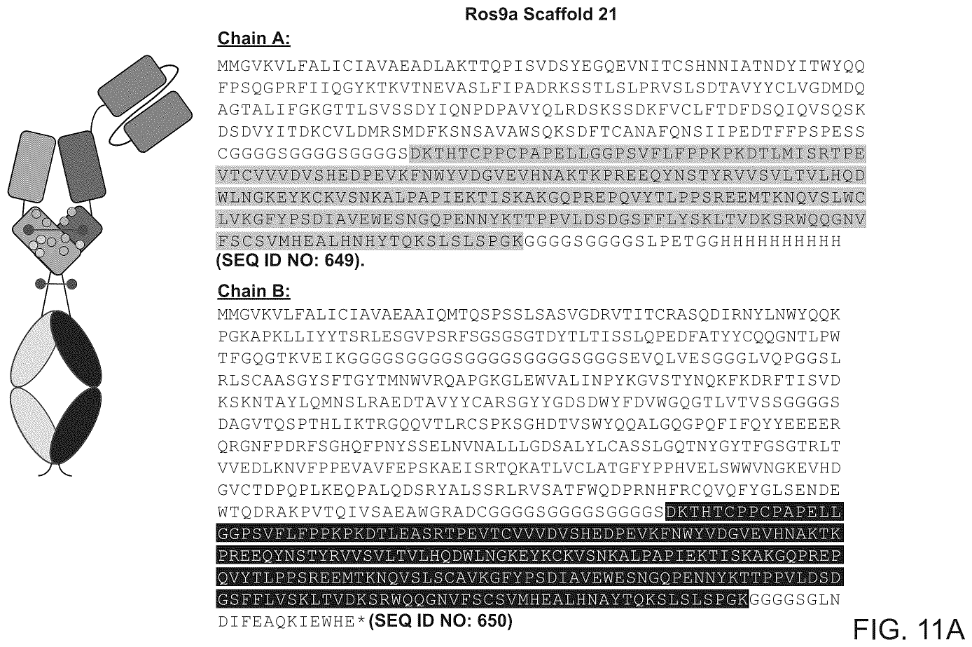

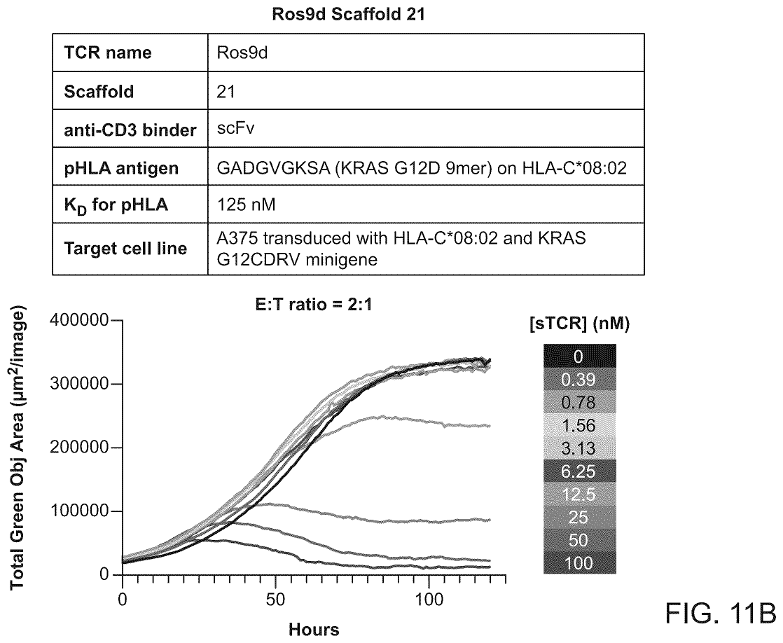

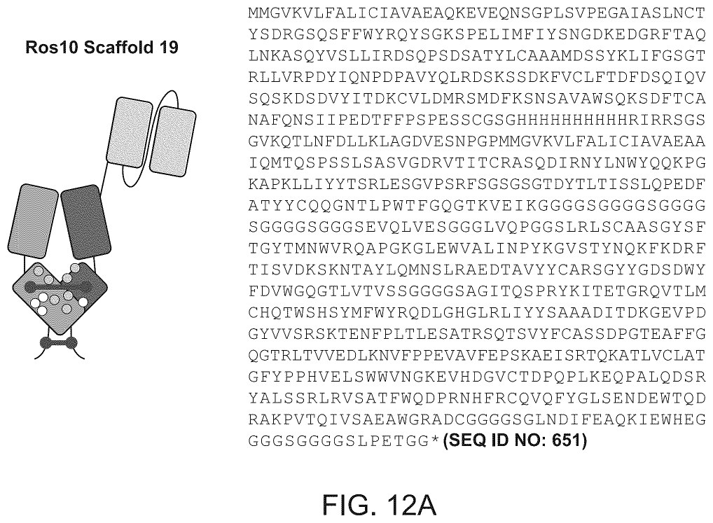

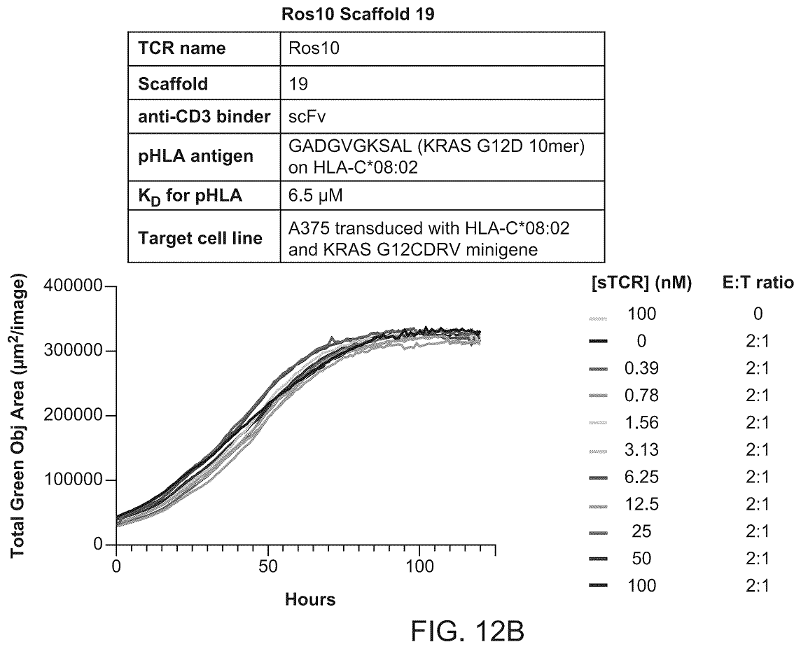

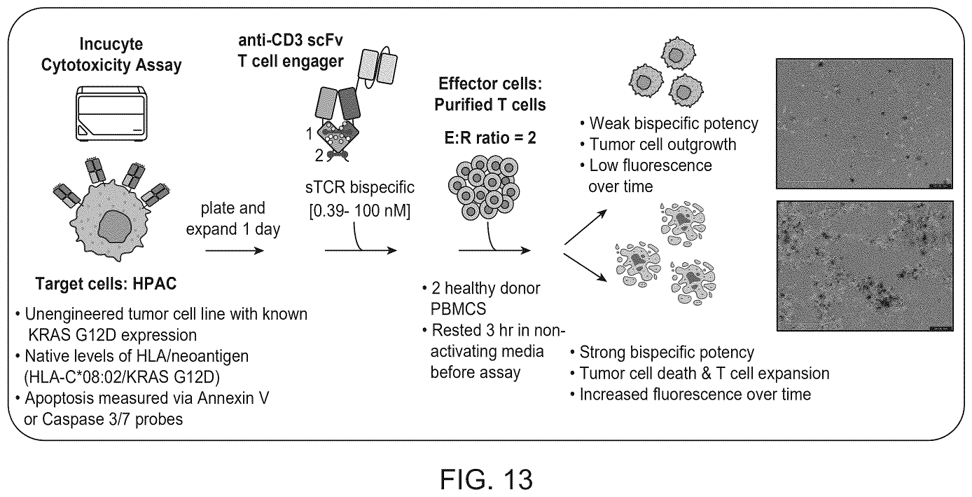

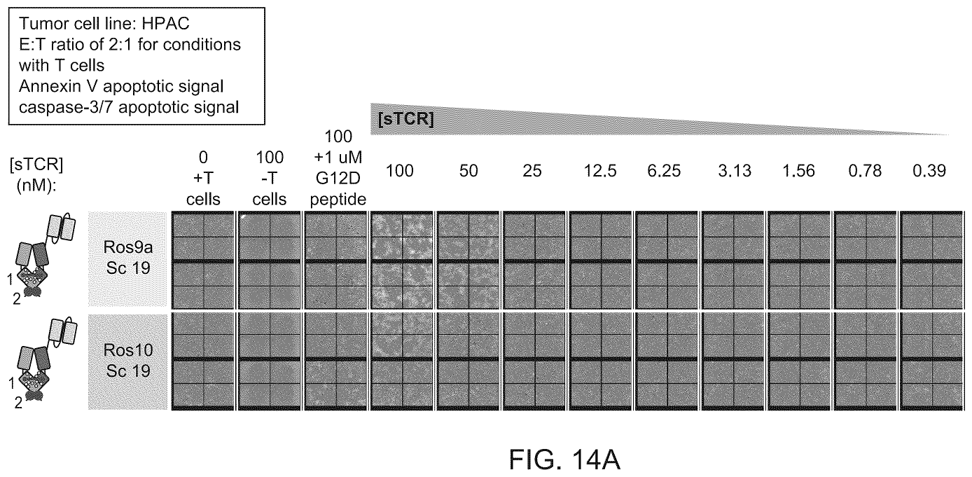

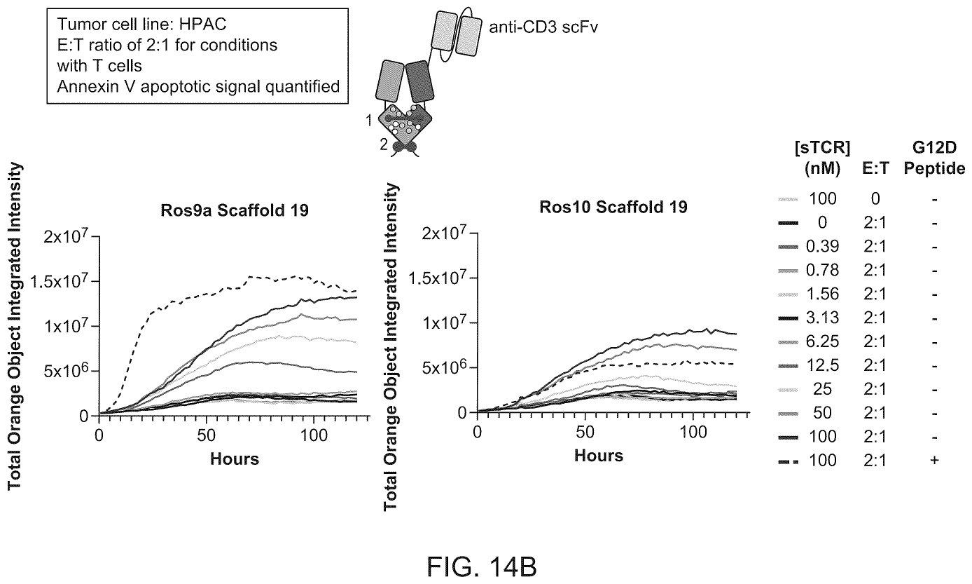

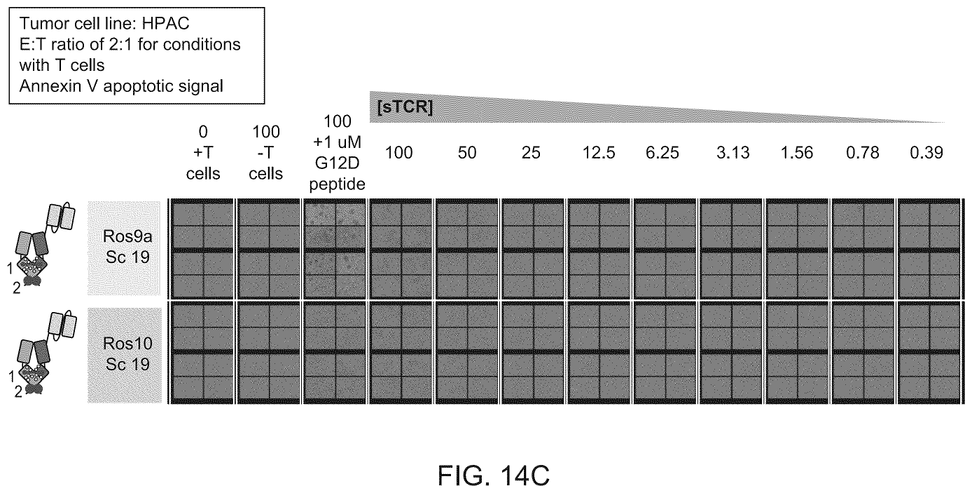

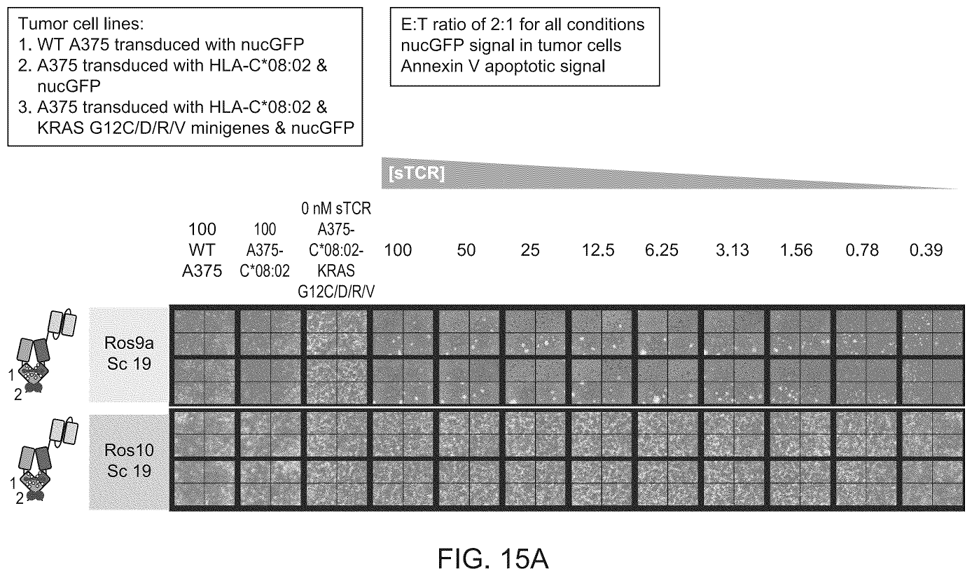

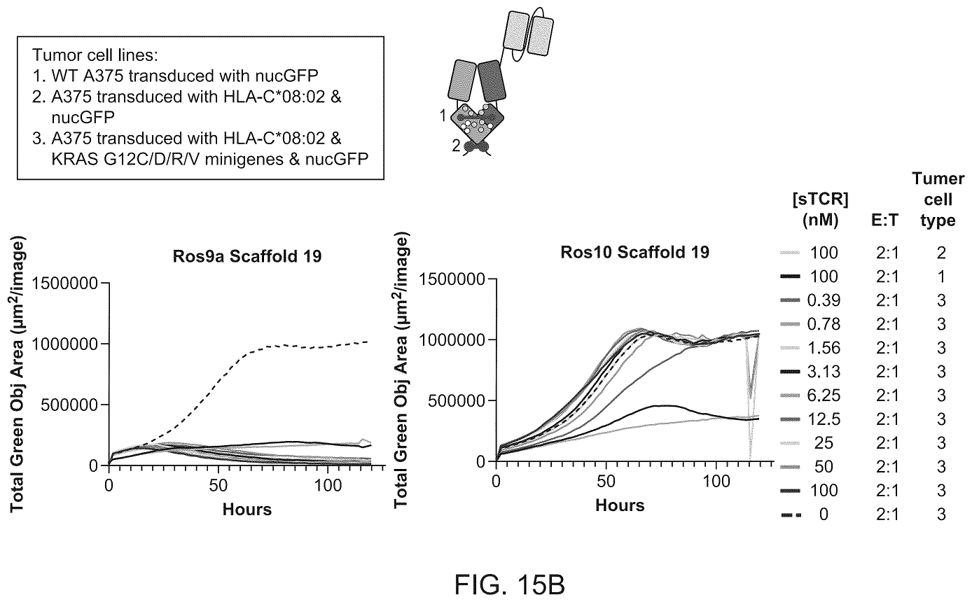

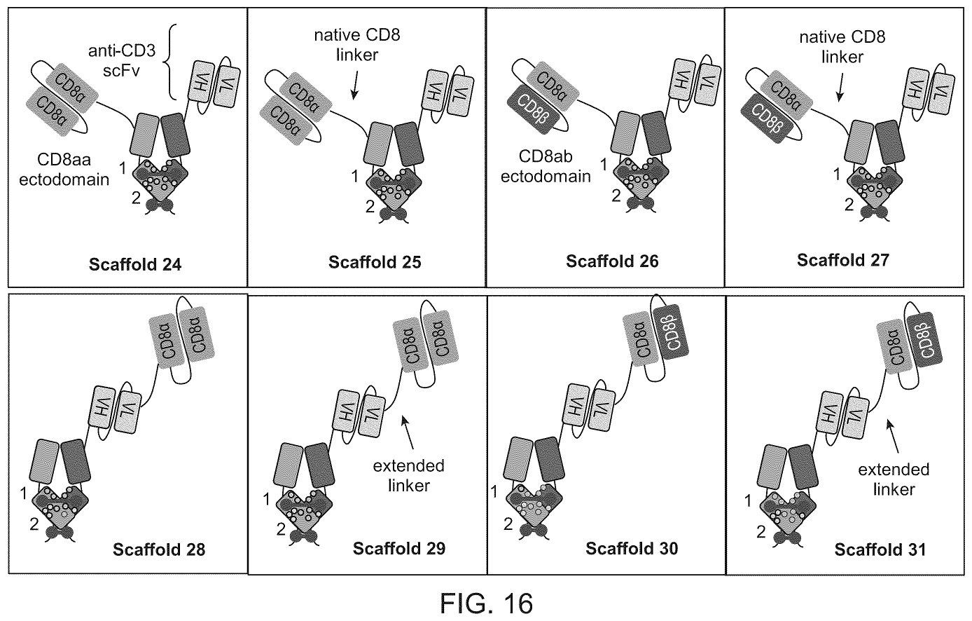

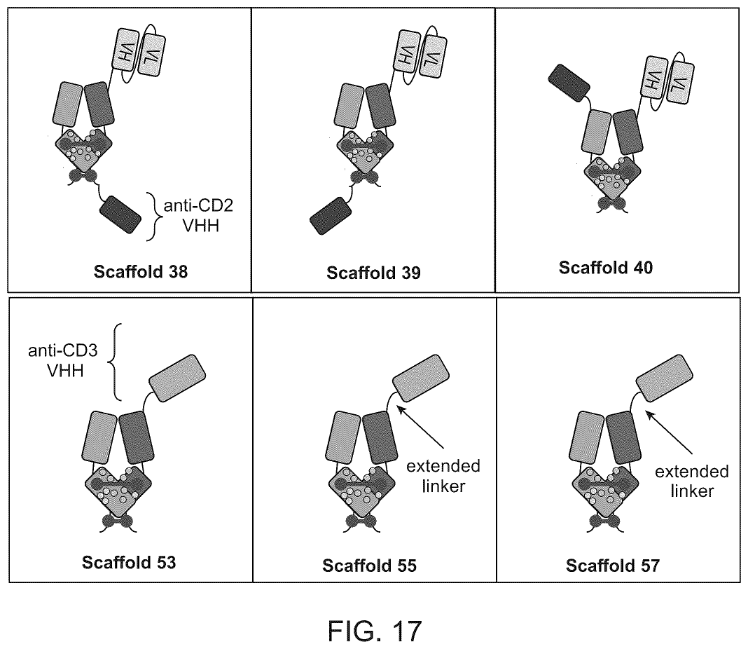

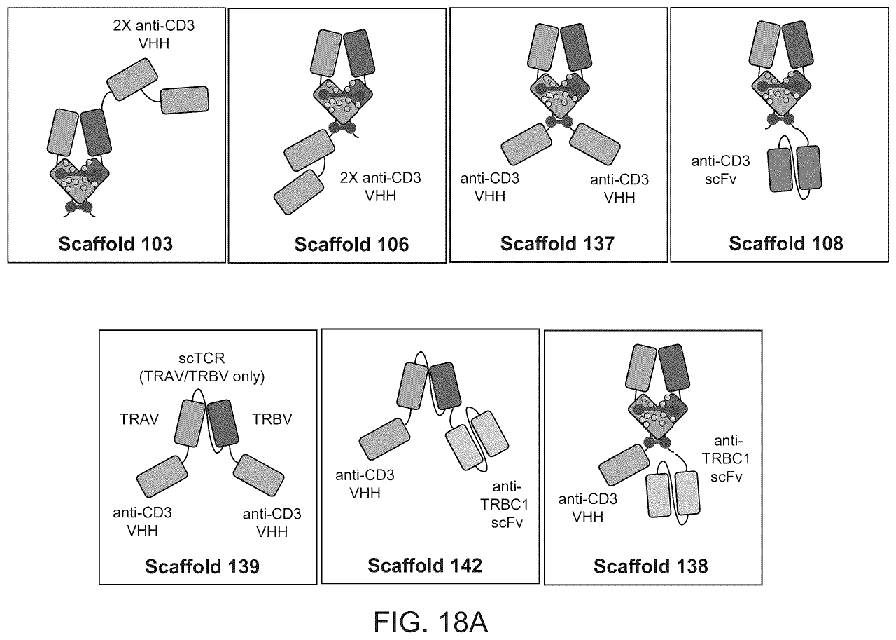

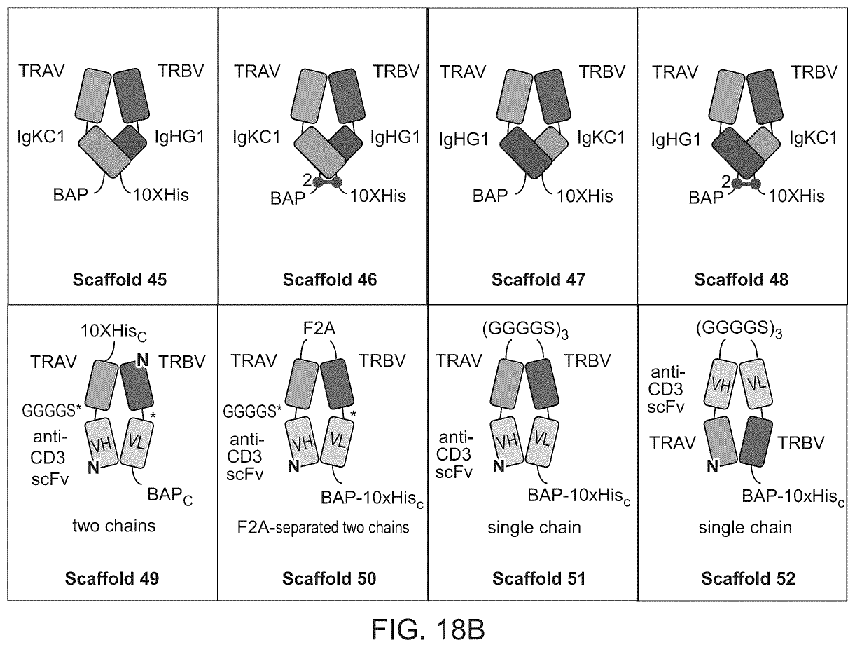

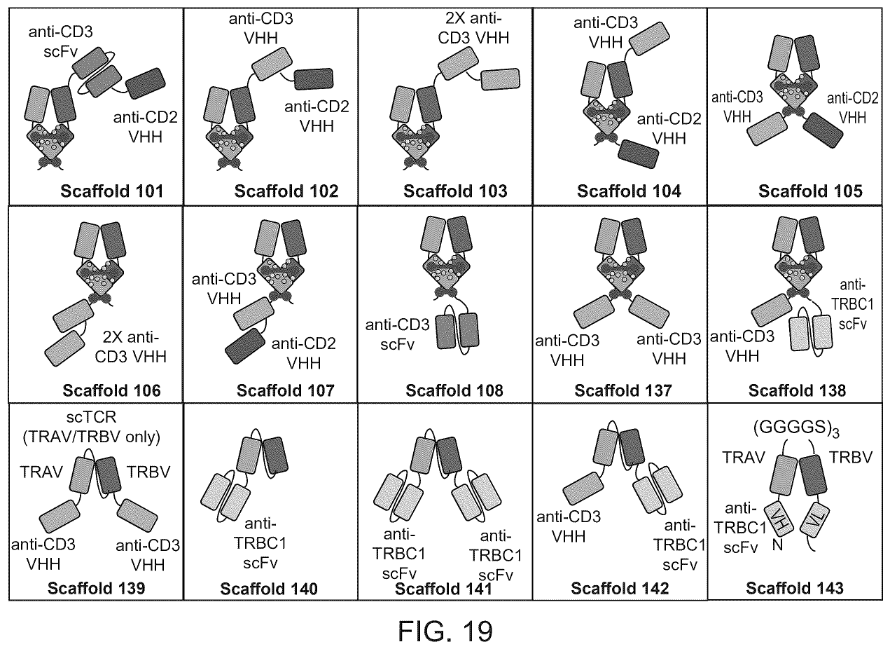

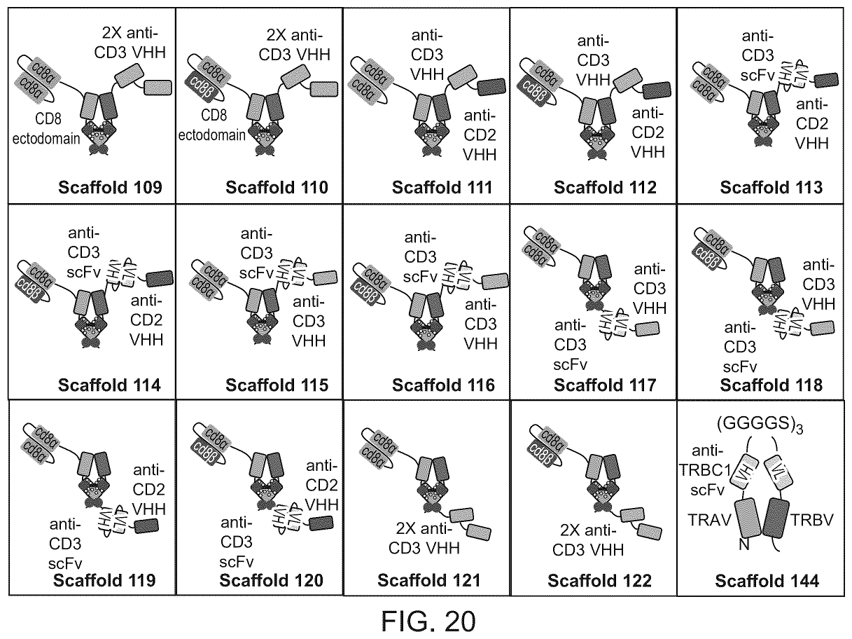

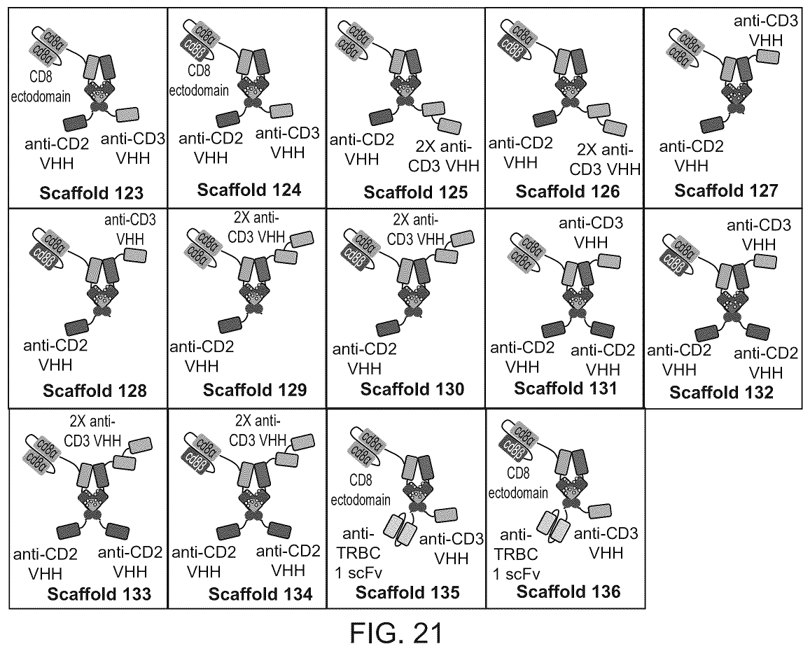

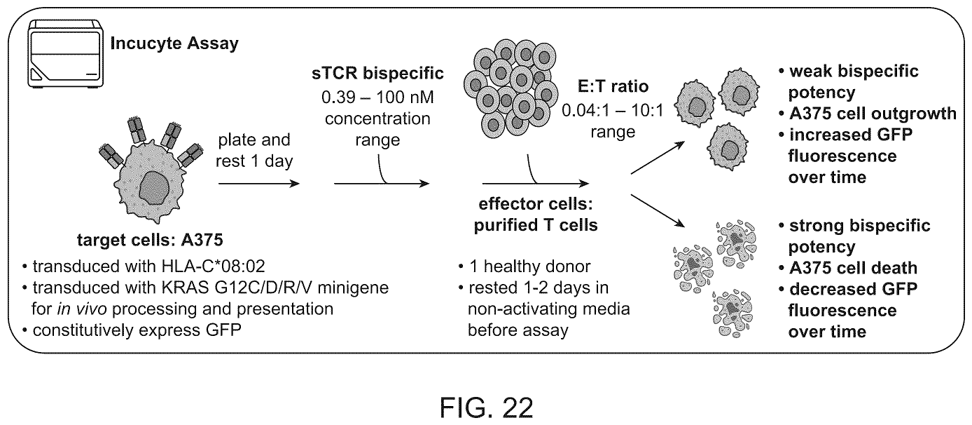

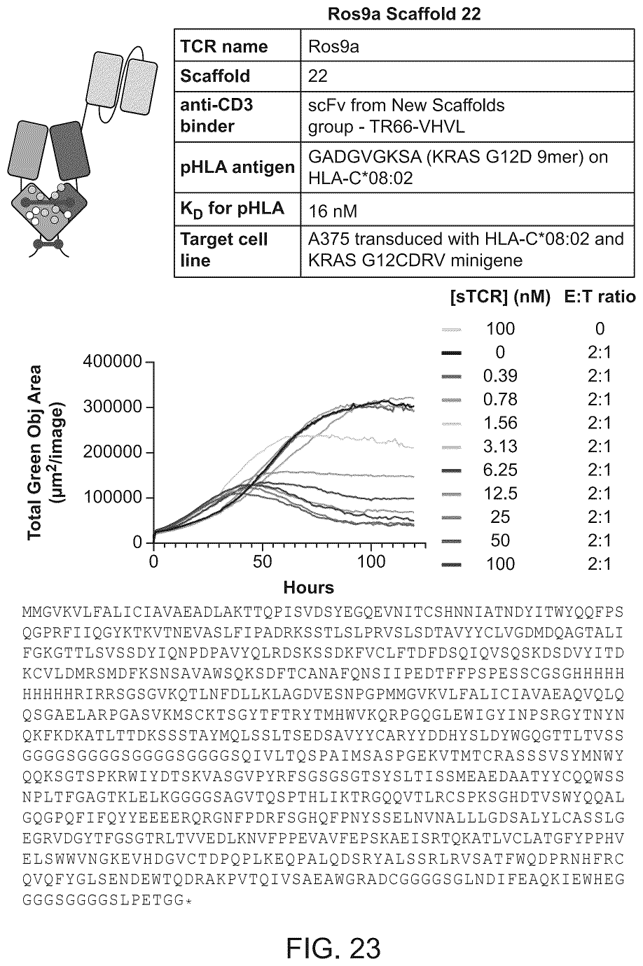

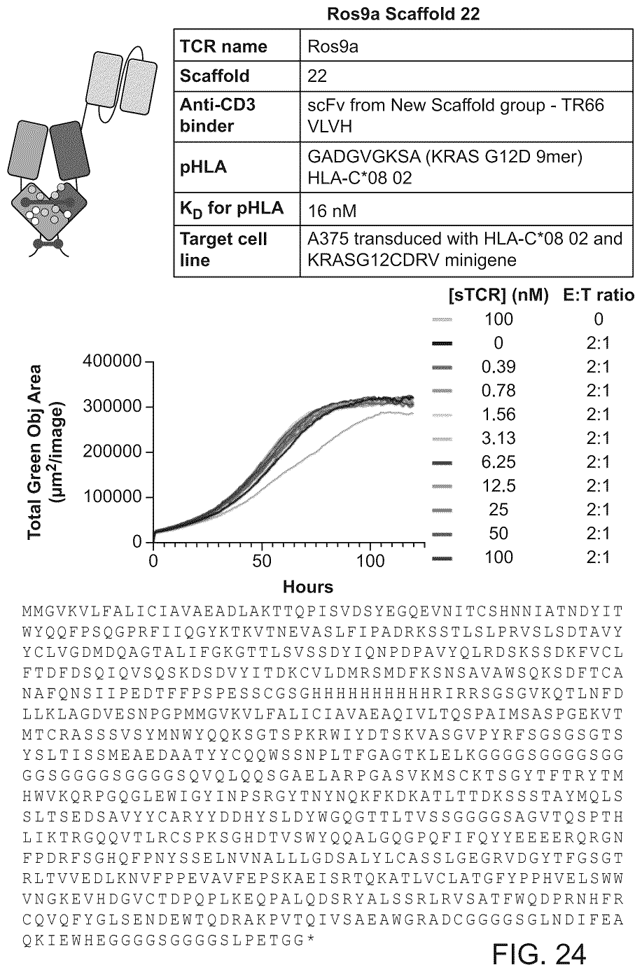

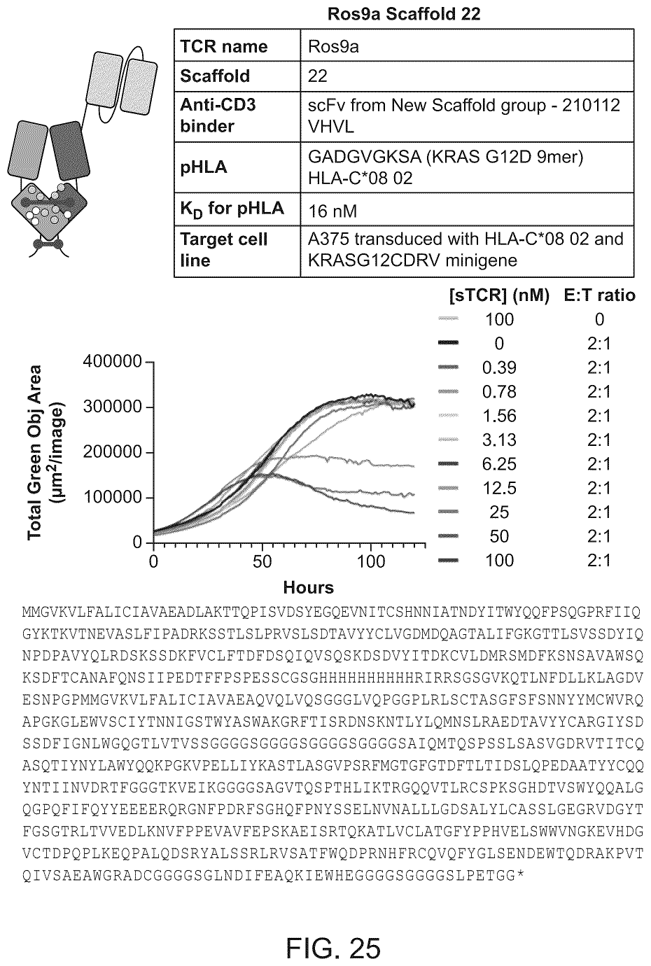

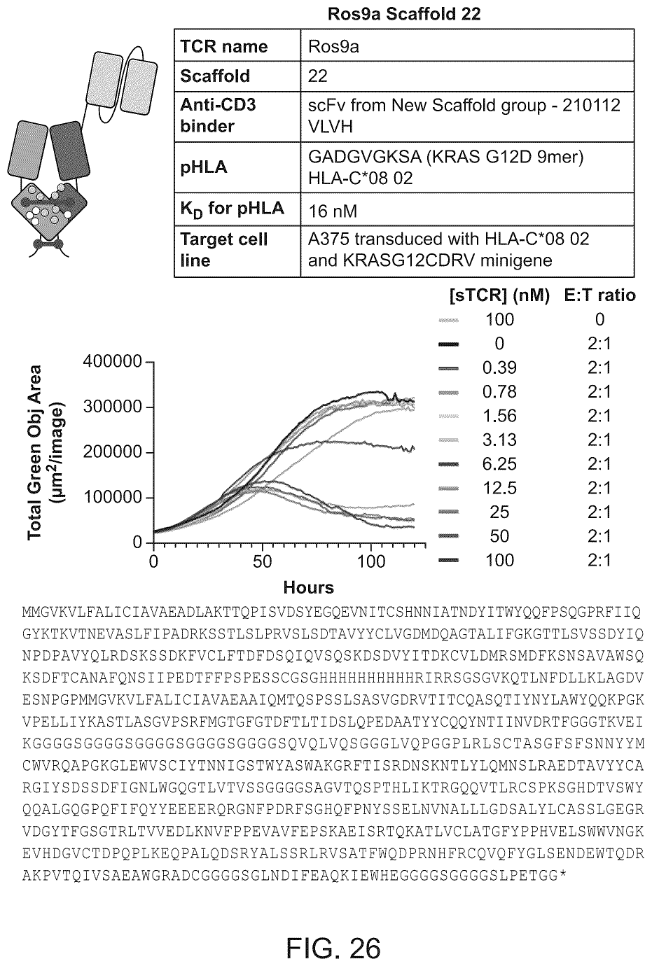

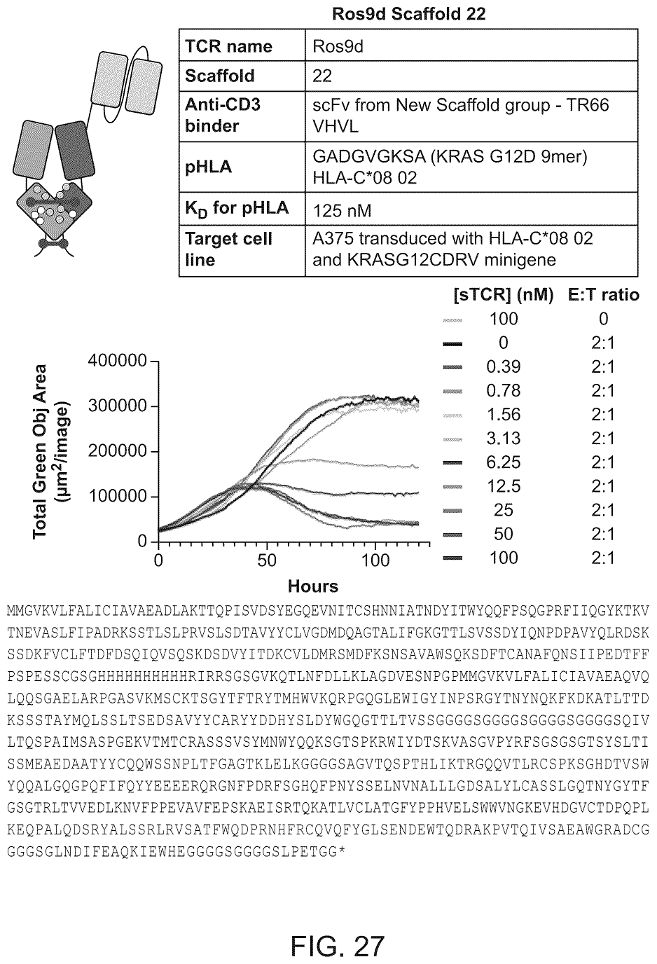

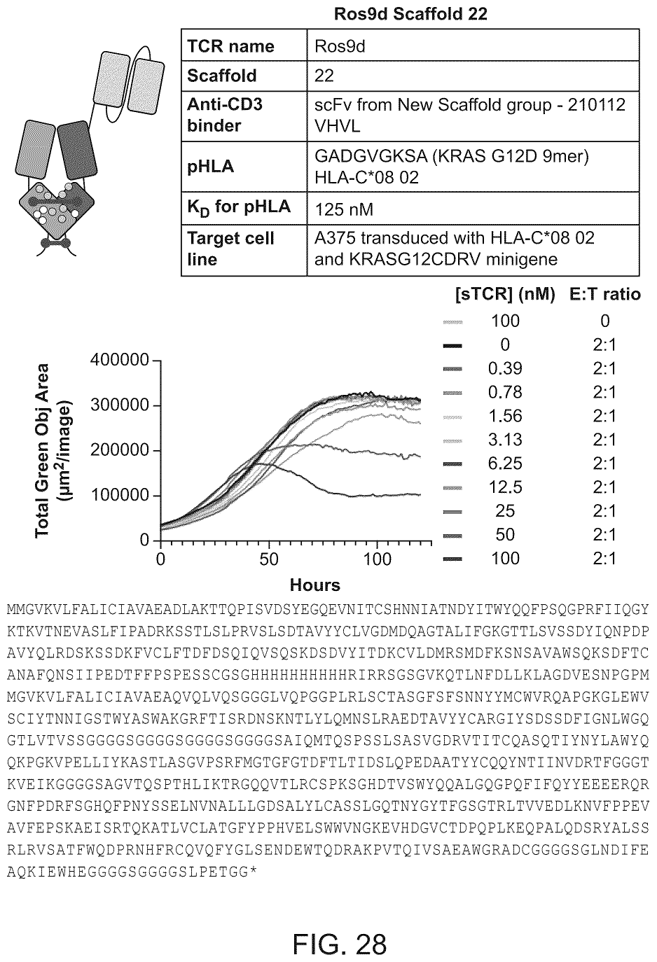

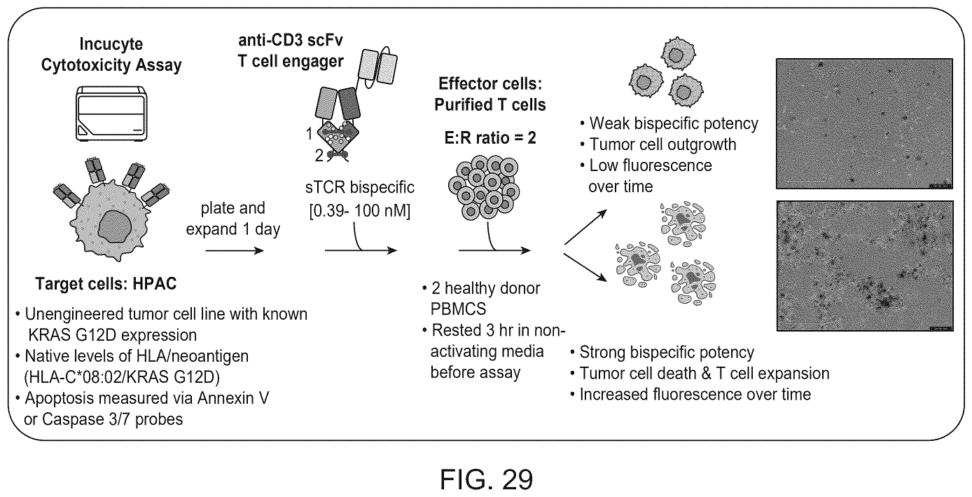

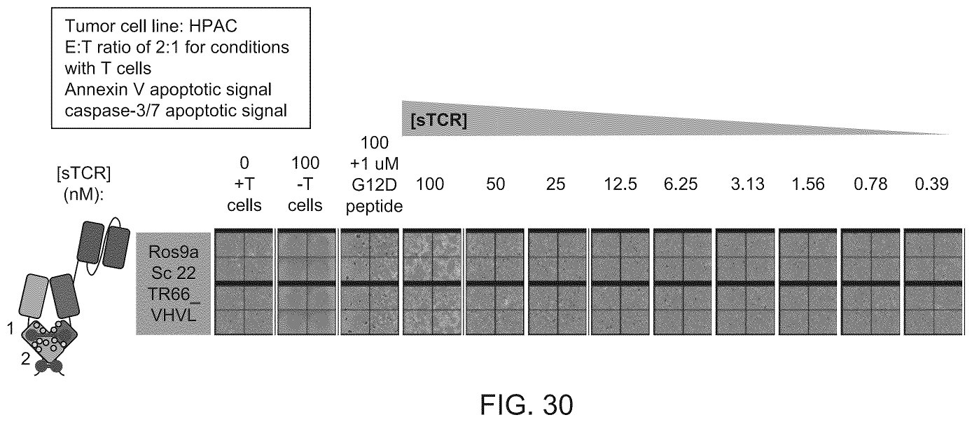

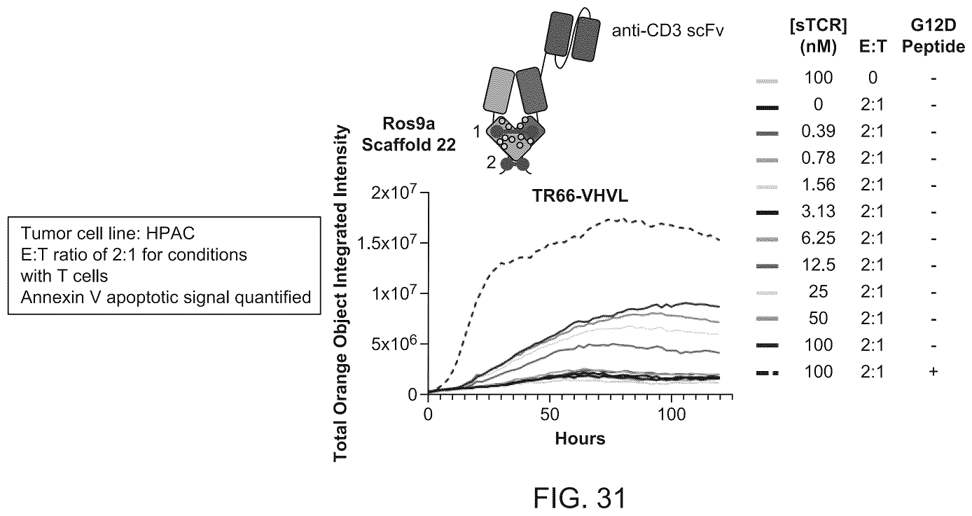

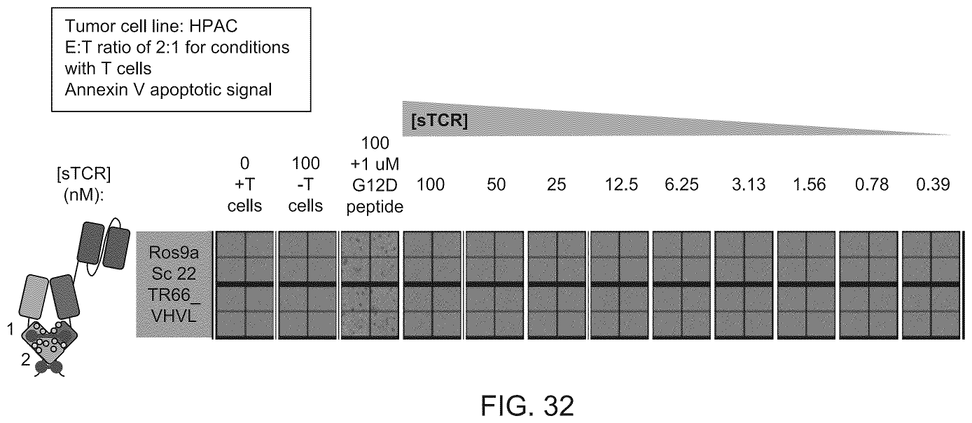

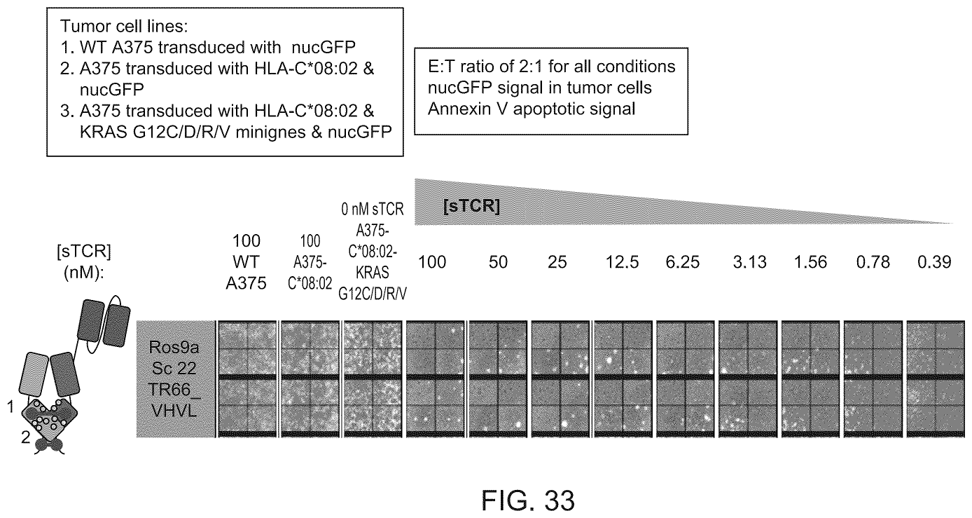

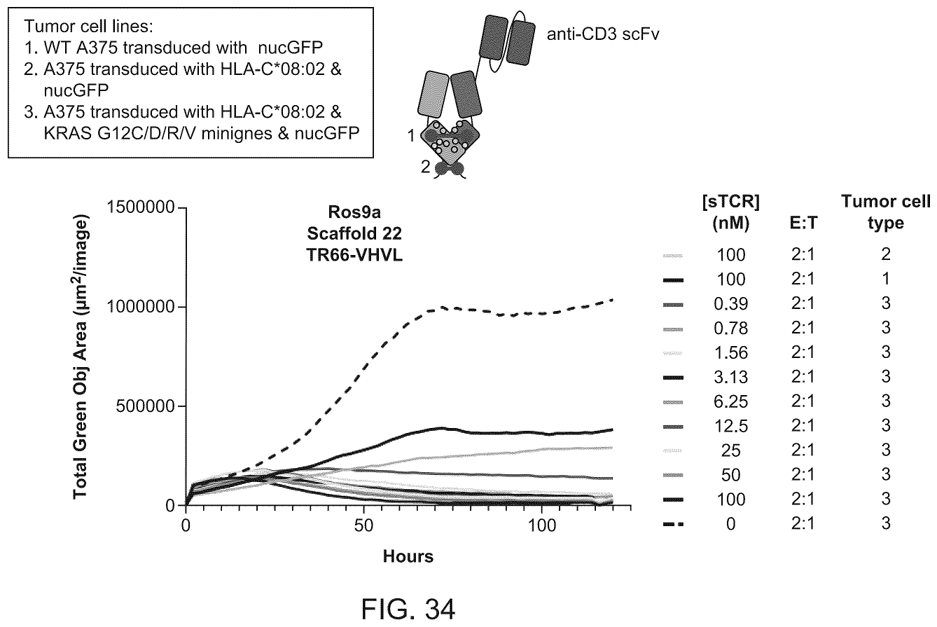

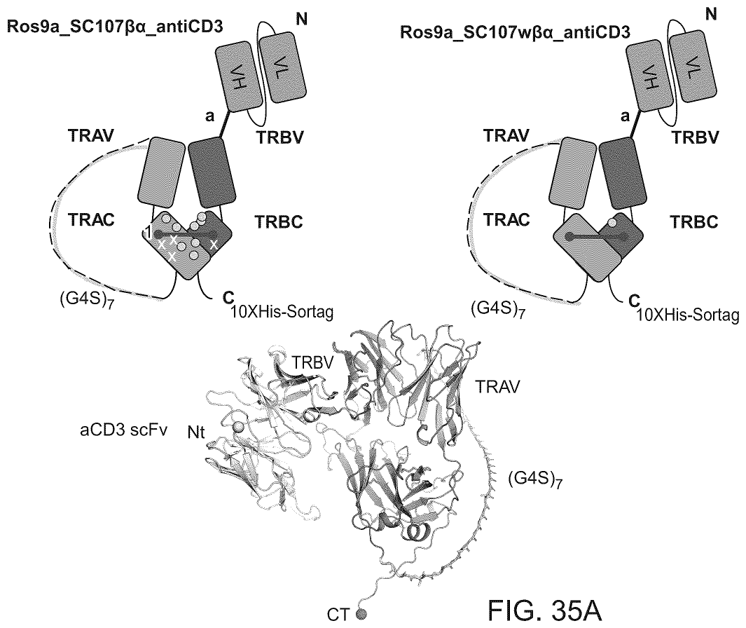

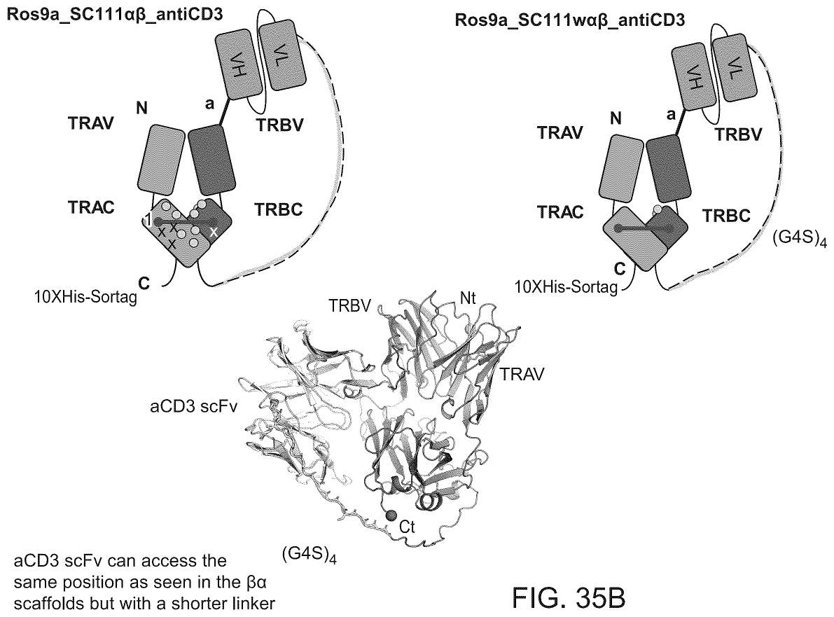

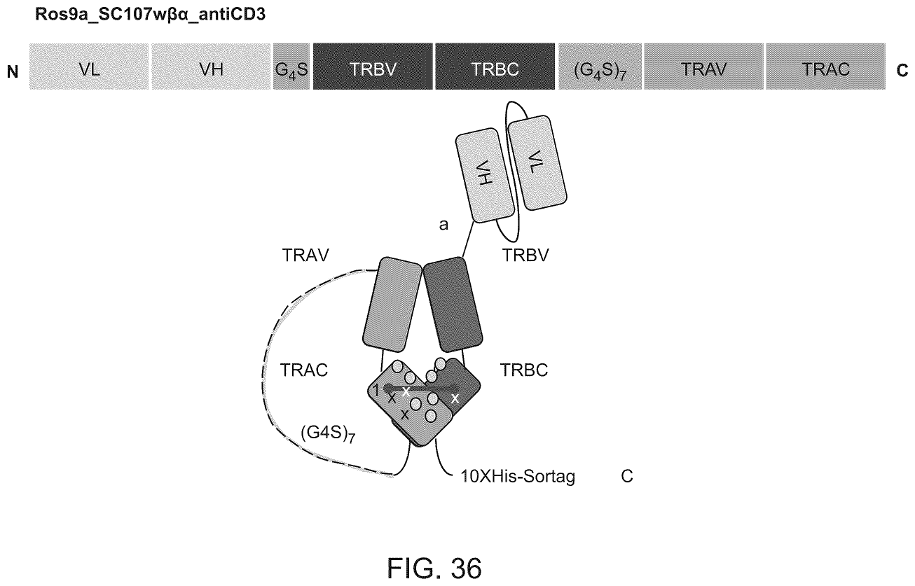

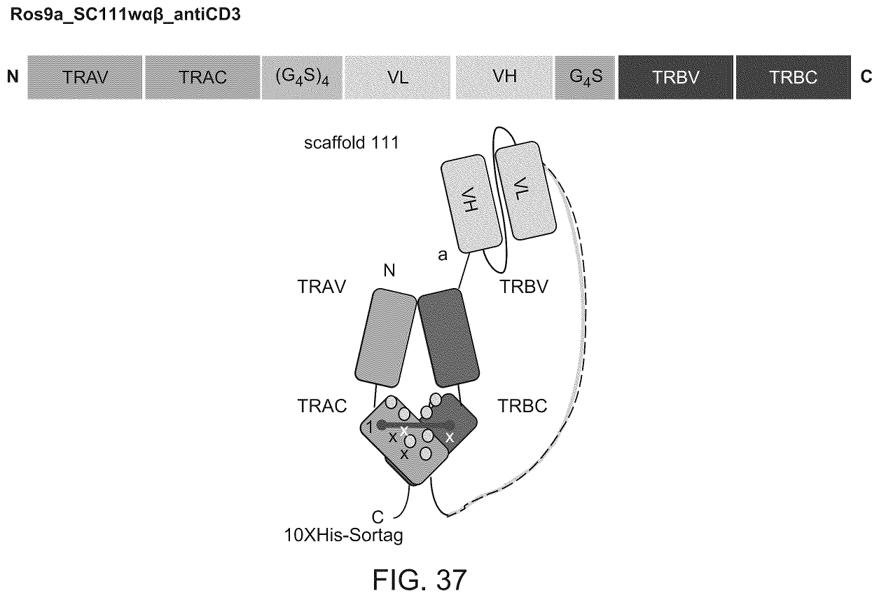

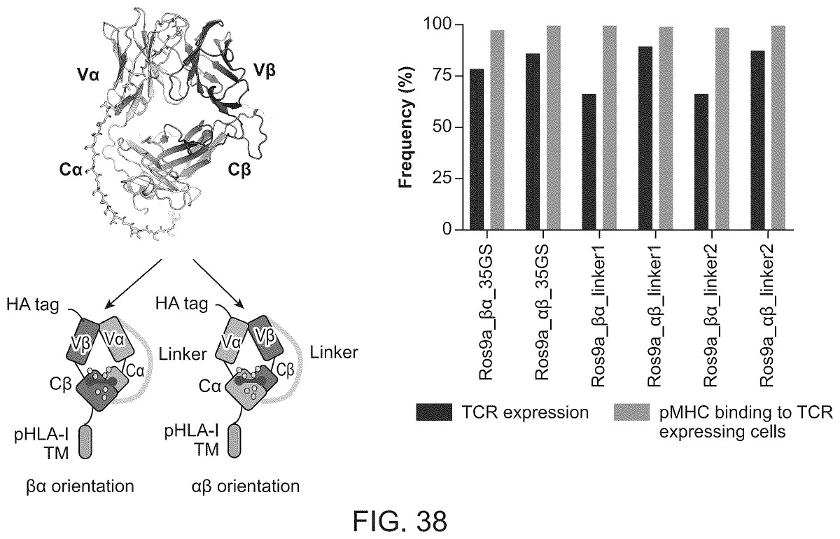

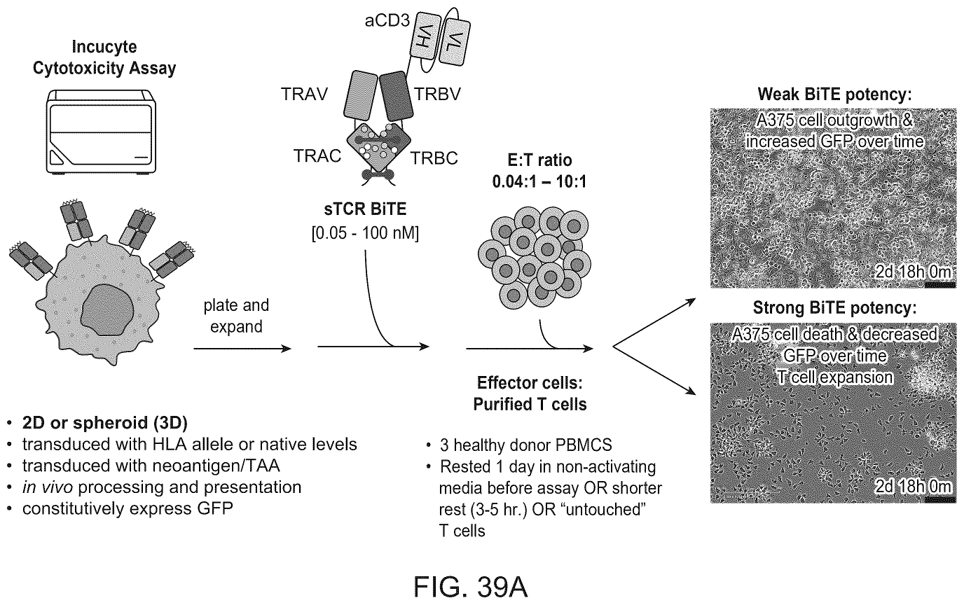

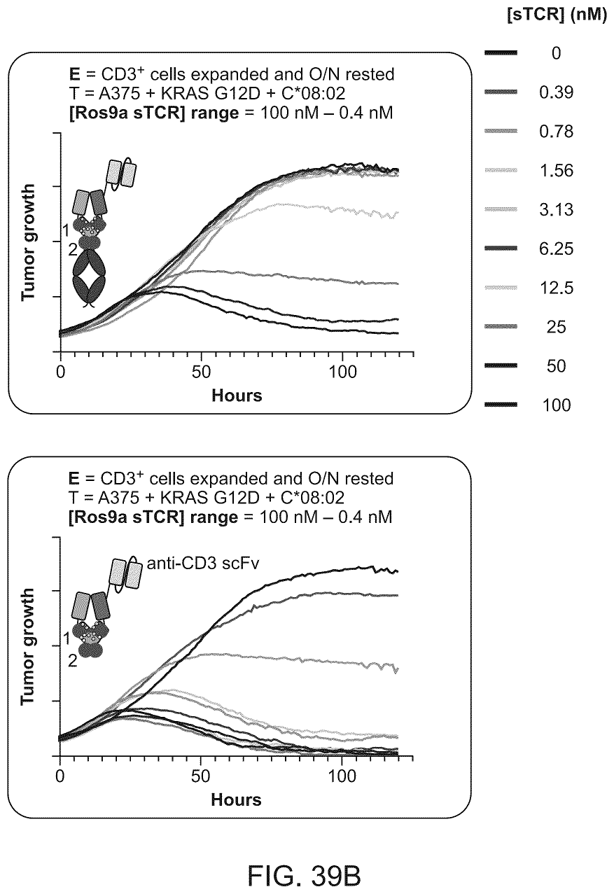

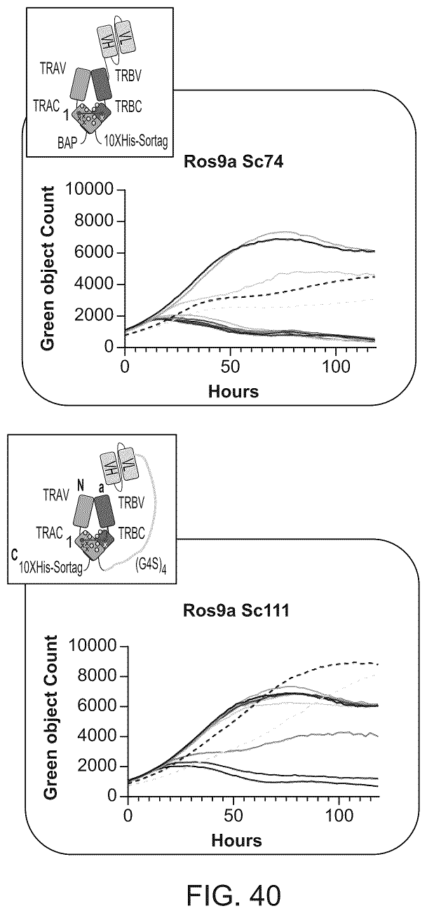

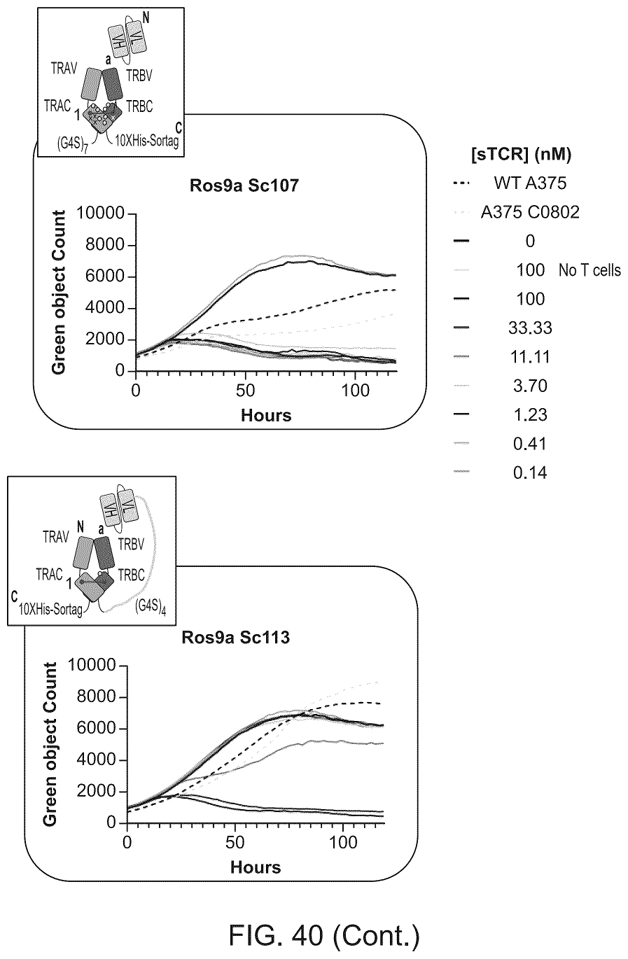

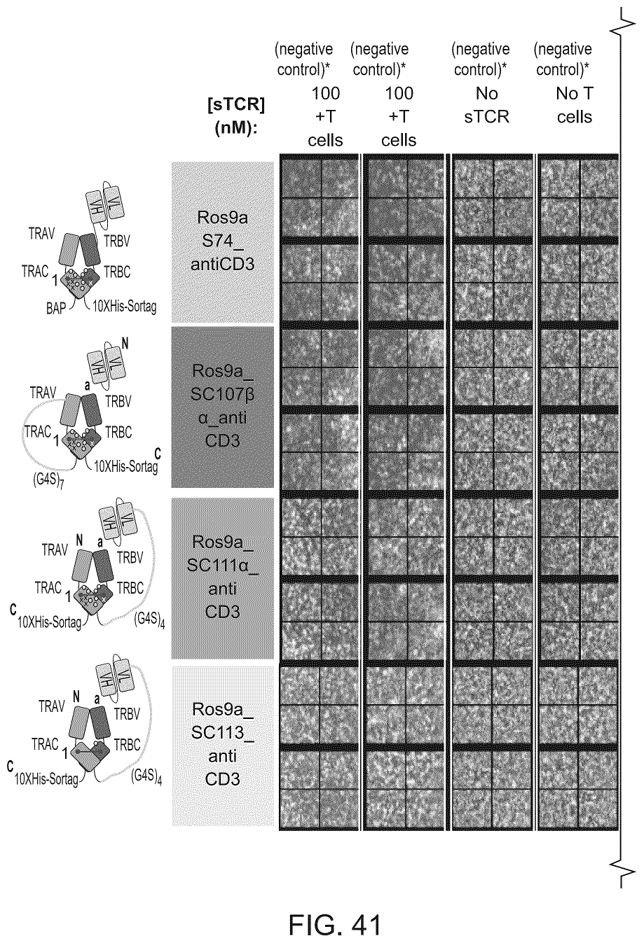

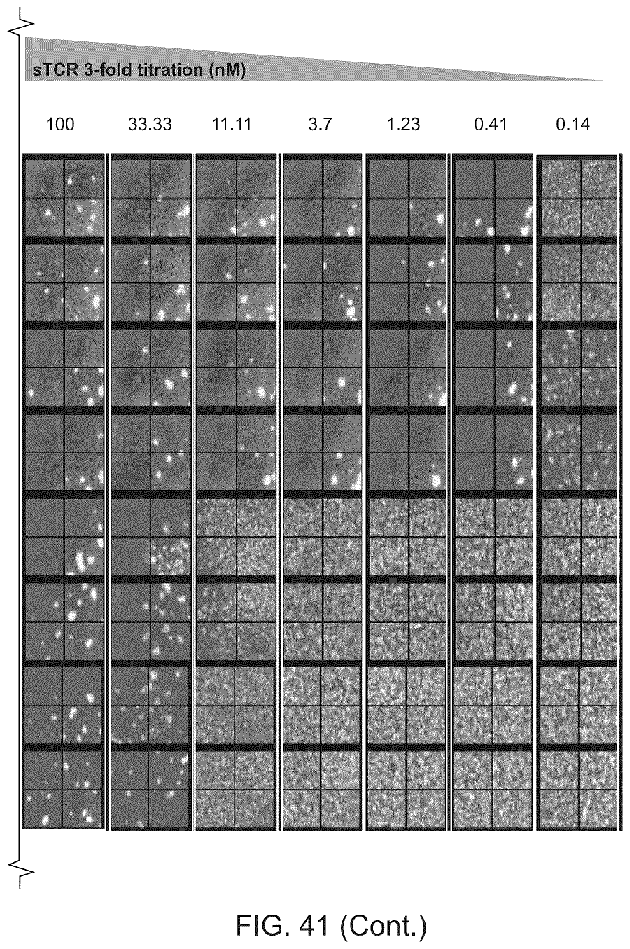

In this aspect, engineered, soluble TCRs could provide a striking advantage. High affinity soluble TCR bispecific or multispecific molecules allow for off-the-shelf precision immunotherapy. Mammalian cell display can identify candidate TCRs with high expression in mammalian cells and native folding, that can be safely used for therapeutic purpose. An exemplary overview of the workflow for sTCR generation is provided in FIG. 1A-1D. Additionally, it allows for the opportunity for novel formulation. The method described herein is designed to generate enhanced TCR platform that circumvents engineering of individual TCRs and enables a plug-and-play use of any functional TCR, combining two basic ideas, enhancing TCR/MHC binding by a bivalent CD8 co-receptor; increasing binding affinity to T cells by bivalent or multivalent binding. The method engages a rational structure-guided and scanning guided mutagenesis of CDR3ab (CDR3 alpha and CDR3 beta) loop positions for enhancing the TCR affinity, which significantly reduces the search space to explore, aim to preserve CDR3 positions that provide strong peptide. HLA (pHLA) recognition and mutagenize weak/neutral positions to enhance affinity and not compromise specificity.

Developing technologies to stabilize and enhance the activity of a soluble TCR outside of the cell could unlock new therapeutic modalities and expand access for patients.

### Multispecific T Cell Engaging Molecules

An attractive and promising alternative to TCR based therapy is the redirection of native T cells to target tumor cells using carefully and rationally designed soluble TCRs. The present disclosure provides a method and compositions for soluble TCRs (sTCRs) to specific antigens and engaging a T cell of modest antigen specificity to generate a robust activation and antigen-specific cytotoxicity. In one aspect, the methods and compositions provided herein allow time and cost-effective method for generation of personalized antigen specific sTCRs. T cell engaging molecules can be engineered to enhance their affinities, a process caused affinity maturation. Even though TCR domains can be matured via phage and yeast display, these techniques share the disadvantages of non-human glycosylation patterns and the need for a later reformatting into the final bispecific format. More importantly, they require very large display libraries (usually >10¹¹ variants) in order to successfully select specific TCRs with high affinity and stability. The combination of antigen recognition and T cell engaging domains enables polyclonal activation of T cells independently of their TCR specificity, or independently of the affinity of the p-MHC: TCR of the T cell. Arguendo, a T cell in close proximity of a target cell in a biological system is likely to possess a minimum, even if transient binding potential to the antigenic peptide of the target cell, sTCRs of the present disclosure can facilitate a prolonged binding and activation for the T cells in proximity of their target cells (especially, where the desired target offers low affinity p:MHC complex for the natural TCR engagement by the T cell), thereby transforming the T cell to an active effector T cell.

One of the attractive concepts disclosed herein is the “plug and play” approach, creating a platform design for customable engager specificities, catering to, but not limited to, for example, personalized immunotherapy using the sTCRs. By generating sTCR scaffold(s) that offers effective structural properties for plugging in TRAV:TRBV domains of interest, or for example, incorporating the sequences for the CDR1, CDR2 and CDR3 of the alpha and beta variable domains directed to any antigen of interest and effectively designed for human therapeutic use, the methods and compositions described herein offers such a platform.

The instant disclosure provides a method that employs activation of a T cell by soluble recombinant TCRs (sTCRs), the sTCRs described herein. The sTCRs disclosed herein are designed to comprise (I) MHC-peptide complex engager, (used interchangeably herein with the term MHC engagers), and (II) T cell engagers, that together not only brings an antigen bearing target cell (e.g., the peptide-MHC bearing cell, e.g., the target cell) and an effector T cell in close proximity by structurally engaging with both and bridging across the two cells, thereby increasing the interaction, but also activates T cell surface molecules (T cell endogenous receptors and coreceptors) that can activate the T cell towards a target specific response. An effector T cell may be a T cell with an endogenous TCR specific for the peptide (e.g., a cognate TCR for the peptide-MHC presented by the antigen bearing target cell). An effector T cell may be a cytotoxic T cell. An effector T cell may be a CD8+ T cell. The antigen bearing target cell may be a cancer cell. A target specific response may be a cytotoxic response.

In one aspect, provided herein is a recombinant multispecific protein molecule, a recombinant sTCR comprising at least two binders or engagers, (i) MHC-peptide complex engager, capable of binding a complex that comprises a specific antigenic peptide in complex with an MHC for antigen presentation. (ii) T cell engager that binds to an extracellular domain of a receptor (or a cell surface ligand) expressed by a T cell. The designer recombinant sTCRs described herein can have a variety of target specificities based on the sequence on the binding domains, in particular, the TRAV and TRBV sequences of the MHC-peptide complex engager, and the sequence of the T cell engager domain. Each of the TRAV and TRBV domains comprise hypervariable complementarity determining regions (CDR) 1, 2, 3 (CDR1, CDR2, CDR3). The instant disclosure provides a method that employs rational structure-guided and scanning-guided mutagenesis of only CDR3αβ loop positions, which not only can significantly reduce the search space to explore but also preserve CDR3 positions that provide strong p-HLA recognition and mutagenize weak/neutral positions to enhance affinity and not compromise specificity. The specificity of the T cell engager is conferred by a variable sequence that may be an antibody or fragment thereof, an scFV or a VHH domain, characterized by the presence of CDR1, 2, and 3 hypervariable domains.

The instant disclosure provides a method that incorporates yet another advantage. The existing methods employ preparing soluble TCRs (sTCRs) in non-mammalian cells, e.g., generate sTCRs in bacterial cells. The instant method is performed in mammalian cells, including recombinant nucleic acid and vector design optimized for expression in mammalian cells, thereby retaining the glycosylation and other post-translation signature conferring adequate expression and activation in a mammalian environment. Post-translational modifications include phosphorylation, glycosylation, ubiquitination, nitrosylation, methylation, acetylation, lipidation and proteolysis and influence cellular functions of the protein, including expression and activity. Furthermore, glycosylated and acetylated peptides have similarly been shown to become more immunogenic in similar studies. In some embodiments, the multispecific engager molecule is generated in a mammalian cell ex vivo in a manufacturing scale. The recombinant nucleic acid is incorporated in mammalian cells in culture, ex vivo, and engineering and/or manufacturing scale quantities of the multispecific engager protein molecule are isolated, harvested and purified. In some embodiments, the multispecific engager molecule comprises a mammalian post-translational modification signature at the time of isolation. In some embodiments, the mammalian cell is a human cell, e.g., a human cell line, cultured in vitro. In some embodiments, the multispecific molecule comprises a post translational modification. In some embodiments, the multispecific molecule comprises a mammalian glycosylation signature. In some embodiments, the multispecific molecule comprises a mammalian phosphorylation signature. In some embodiments, the multispecific molecule comprises a mammalian acetylation signature.

Accordingly, provided herein is a composition comprising a recombinant nucleic acid comprising a sequence encoding a multispecific molecule, wherein the multispecific molecule is an engineered T cell receptor (TCR) construct, comprising: (i) an MHC-peptide complex engager comprising: (a) a T cell receptor (TCR) alpha variable (TRAV) domain, and (b) a TCR beta variable (TRBV) domain; and (ii) a T cell engager comprising a binding domain that binds to an extracellular domain of a T cell receptor, or a T cell ligand, wherein the multispecific molecule comprises a post-translational modifications of a human cell.

In some embodiments, the recombinant multispecific molecule is soluble. A soluble protein molecule may be one that is not incorporated in a cell membrane, or may not be localized intracellularly in its mature form. For example, the multispecific molecule lacks a trans-membrane domain. In some embodiments, the multispecific molecule may be secreted from a cell.

In some embodiments, the multispecific molecule comprises two polypeptides, e.g., a first polypeptide and a second polypeptide. In some embodiments, the first polypeptide and the second polypeptide comprise one or more multimerization domains or stabilization domains, that essentially hold the two polypeptides together in a structural assembly. In some embodiments, the first polypeptide and the second polypeptide comprise one or more disulfide bonds between the two polypeptides.

In one aspect, the disclosure provides one or more synthetic or recombinant biomolecules, such as proteins or polypeptides, that are capable of binding to and activating a T cell to trigger an immune response against a target cell, such as a tumor cell. In some embodiments, the synthetic or recombinant biomolecule can bind (a) on one hand, a cell surface molecule (i.e. and antigen) in the context of MHC complex on a target cell, and on the other hand (b) a cell surface molecule (i.e. and antigen or a receptor) on a T cell, thereby effectively at least bringing the two cells (a target and an effector cell respectively), in close proximity, such that other cellular receptors and membrane components on either cell can interact and the effector T cell can thereby trigger killing of the target cell. Such synthetic or recombinant biomolecules can be called bispecific engagers, or, bispecific T cell engagers, or BiTEs. In one or more embodiments, the bispecific engagers comprise two antigen binding domains (“binders”). At least one of the two binders is designed to bind to a protein expressed on the surface of an effector T cell; and at least one of the two binders is designed to bind to an antigen peptide-MHC complex on a target cell. In some embodiments the antigen binding domains are antibodies or fragments thereof. In some embodiments, a binder may be a ligand, binding to a receptor on a cell surface, such as a receptor on a T cell or on a target cell.

In one aspect, the present disclosure provides a therapeutic composition comprising one or more synthetic or recombinant biomolecules, such as proteins or polypeptides, that are capable of binding to and activating a T cell to trigger killing and immune response against a target cell, such as a cancer cell, and the synthetic or recombinant biomolecule comprises more than two binders. Accordingly, in some embodiments, provided herein is a therapeutic agent, wherein the therapeutic agent comprises: a first binding domain (or, a first binder), wherein the first binding domain may be a first TCR antigen binding domain or functional fragment thereof that specifically interacts with an antigen or a surface molecule in the context of peptide-MHC complex on a target cell, and a second binding domain (or, a second binder), wherein the second binding domain may an antibody or functional fragment, a ligand or a receptor or that specifically interacts with a T cell.

In one aspect, the recombinant biomolecule comprises three binders, each of which exhibit specific binding to a surface molecule, and therefore the recombinant biomolecule can exhibit binding to three elements on two or more cells. In one embodiment, the recombinant biomolecule having three binders is capable of binding to more than one antigens on a T cell or on a target cell. A recombinant biomolecule as described here, having three binders is termed a trispecific T cell engager (TriTE). In some embodiments, a TriTE may bind to, or engage two or more different cells, for example, at least one T cell, and at least one target cell such as a cancer cell. In some embodiments, the BiTE or TriTE may engage more than one antigens or surface molecules on either a T cell or on a cancer cell that either activates the T cell or inhibits a function of a cancer cell. In some embodiments, a bispecific, trispecific or a multispecific engager may comprise a second trigger, i.e., a second signal that not only induces killing of the target cell by the T cell, but also initiates an immune response or inflammatory response that activates other immune cells for a prolonged response and generation of immunological memory. In some embodiments, a bispecific, trispecific or a multispecific engager is a chimeric molecule.

Using the methods and compositions described herein, a T cell can be directed to activate the immune response cycle irrespective of the effects in a tumor microenvironment. A T cell can be directed to kill a target cell and activate the immune response sequelae that generates successful and sustained adaptive immune response and immunological memory against the target.

The instant disclosure provides methods and compositions for in vivo use. In some embodiments, provided herein is a method for in vivo administration of a recombinant nucleic acid construct encoding a soluble TCR described herein.

In the following section, compositions comprising therapeutic agents are described.

### I. MHC-Peptide Complex Engager

Significant progress has been made in identifying tumor specific antigens that can elicit immune response. Tumor-specific antigens (TSAs) may be neoantigens. Neoantigens may be unique to cancer cells and absent in normal tissues, for example in cancer vs. non cancers tissues within a single human subject, making them attractive targets for immunotherapy. Although the vast majority of somatic mutations are random passenger mutations that are unique to an individual patient, a subset of mutations in oncogenic driver genes such as KRAS, PIK3CA or ERBB2 and tumor suppressors including TP53 and PTEN form a class of shared antigens common amongst patient cohorts. Being both immunogenic and restricted to a common HLA, some of these shared antigens may commonly be considered driver mutations. Examples of currently described shared antigens include KRAS G12D/G12V, collectively found in 60-70% of pancreatic adenocarcinomas and 20-30% of colorectal cancers, and PIK3CA H1047L, detected in about 5% of metastatic breast cancers. High affinity soluble TCR specifically targeting such shared common antigens allow for off-the-shelf precision immunotherapy for cancer patients to be possible.

Provided herein is a composition comprising a recombinant nucleic acid comprising a sequence encoding a multispecific molecule, e.g., a multispecific engager molecule, wherein the multispecific molecule comprises: (i) an MHC-peptide complex engager comprising: (a) a T cell receptor (TCR) alpha variable (TRAV) domain, and (b) a TCR beta variable (TRBV) domain; wherein the peptide in the MHC-peptide complex comprises a cancer specific peptide, such as a RAS peptide sequence; and (ii) a T cell engager comprising a binding domain that binds to an extracellular domain of a receptor expressed on a T cell. Provided herein is a recombinant nucleic acid comprising a sequence encoding a multispecific molecule that may be used in therapy, for use in vivo in a human subject in need thereof, wherein the recombinant nucleic acid comprising the sequence encoding the multispecific molecules is administered to the human subject, and the recombinant nucleic acid is taken up by a cell in vivo, and the multispecific engager molecule encoded by the recombinant nucleic acid is expressed in vivo. In some embodiments, the multispecific engager molecule is administered to the subject, wherein the multispecific molecule comprises a post-translational modifications of a human cell.

In some embodiments, the multispecific molecule comprises a first polypeptide and a second polypeptide, wherein the first polypeptide comprises the TRAV domain and the second polypeptide comprises the TRBV domain.

In some embodiments, the peptide of the MHC-peptide complex is a peptide from a cancer antigen.

In some embodiments, the peptide of the MHC-peptide complex comprises a mutation.

In some embodiments, the MHC in the MHC-peptide complex binds a wild-type peptide of the MHC-peptide complex with a lower affinity compared to the peptide comprising the mutation.

In some embodiments, the peptide of the MHC-peptide complex does not comprise an amino acid sequence YLEPGPVTA.

In some embodiments, the MHC of the MHC-peptide complex comprises a class I MHC.

In some embodiments, the MHC of the MHC-peptide complex is a class I MHC polypeptide.

An MHC of the MHC-peptide complex is encoded by an HLA. In general about six HLA alleles are expressed in every human beings. In general, an allele is described as one of two or more versions of DNA sequence (a single base or a segment of bases) at a given genomic location. An individual inherits two alleles, one from each parent, for any given genomic location where such variation exists. If the two alleles are the same, the individual is homozygous for that allele. If the alleles are different, the individual is heterozygous. The distribution and frequency of HLA antigens vary greatly among different ethnic groups. The genes coding for HLA heterodimers are highly polymorphic, with more than 12,000 class I and 4,000 class II allele variants identified across the human population. It has been postulated that this diversity of HLA polymorphism has evolved under unique selective pressure in different geographic areas. Some HLA alleles are present in higher frequency in a particular human population than others. Accordingly, a particular HLA allele may have higher representation in a given population than another HLA allele. A low frequency HLA allele, for the purpose of discussion may be an allele that occurs as 1% or less than 1% in a given human population (e.g., a human population cohort described by race or geographical area). Reference may be drawn, only for exemplary purposes, to Caucasian population, or to a North American population.

In some embodiments, the MHC of the MHC-peptide complex has a peptide binding affinity greater than at least 50 nM. In some embodiments, the MHC of the MHC-peptide complex binds a peptide antigen that is present in low abundance in a disease system. For example, a cancer antigen may belong to a protein which is expressed in low abundance in a cell, e.g., a cancer cell, but may be an antigen that evokes are strong immunogenic response, e.g., T cell activation. For example, in particular, a low abundance protein is a protein for which fewer copy numbers exists in a cell compared to the usual copy numbers of other proteins of medium or high expression. In one embodiment, the sTCR system may be developed wherein the MHC-peptide complex engager recognizes a low abundance protein in a p-MHC complex.

In full unmodified forms of TCR, the alpha variable domain (Vα) and the beta variable chain domain (Vβ) are separate polypeptide chains, i.e., are located in the α chain and β chain, respectively. Upon interaction of the TCR α chain and β chain, in particular the interaction of the Vα and Vβ domains, one of the epitope binding sites of the TCR is formed.

In some embodiments, the binding domain may further be modified to increase its binding specificity or binding affinity or both. One of skill in the art can use existing technology to enhance the binding properties of a binder region, and such modifications are contemplated within the scope of this disclosure.

In some exemplary embodiments, TRAV:TRBV discovery may follow from a subject-specific cancer-specific neoantigenic epitope analysis: (a) neoantigens may be obtained for a single subject by gene expression analysis on a genome-wide or exome scale; (b) epitope that are differentially expressed in cancer versus non-cancer cells from the subject may be HLA-matched to a strong affinity MHCs encoded by an HLA of the subject using a suitable MHC-peptide prediction algorithm, e.g., RECON 3.0, NeonMHC2, NetMHCpan, IEDB; (c) T cells may then be screened for high affinity binding to the respective subject-specific cancer-specific neoantigenic peptide; (d) TCR variable regions from screened T cells may then be sequenced. TRAV and TRBV sequences thus obtained may be employed to generate recombinant sTCRs of the nature described herein. The generalized method may be modified effectively by using one or more specific improvements described herein. Integration of selected TRAV:TRBV pairs into a suitably well-designed scaffold structure can generate novel and effective sTCRs for in vivo therapy.

In some embodiments, a suitable MHC-peptide prediction algorithm is a the machine learning HLA peptide presentation prediction model. In some embodiments, the machine learning HLA peptide presentation prediction model is trained using training data comprising sequence information of sequences of training peptides identified by mass spectrometry to be presented by an HLA protein expressed in training cells. In some embodiments, the method comprises ranking, based on the presentation predictions, at least two peptides identified as being presented by at least one of the one or more proteins encoded by a class II HLA allele of a cell of the subject. For example, Applicant's earlier filed Application PCTUS201968084, filed by Applicant on Dec. 20, 2019 and published as WO2020132586 on Jun. 25, 2020 provides guidance on the matter and is fully incorporated herein.

In some embodiments, the method comprises selecting one or more peptides of the two or more ranked peptides.

In some embodiments, the method comprises selecting one or more peptides of the plurality that were identified as being presented by at least one of the one or more proteins encoded by a class II HLA allele of a cell of the subject.

In some embodiments, the method comprises selecting one or more peptides of two or more peptides ranked based on the presentation predictions.

In some embodiments, the machine learning HLA peptide presentation prediction model has a positive predictive value (PPV) of at least 0.07 when amino acid information of a plurality of test peptide sequences are processed to generate a plurality of test presentation predictions, each test presentation prediction indicative of a likelihood that the one or more proteins encoded by a class II HLA allele of a cell of the subject can present a given test peptide sequence of the plurality of test peptide sequences, wherein the plurality of test peptide sequences comprises at least 500 test peptide sequences comprising (i) at least one hit peptide sequence identified by mass spectrometry to be presented by an HLA protein expressed in cells and (ii) at least 499 decoy peptide sequences contained within a protein encoded by a genome of an organism, wherein the organism and the subject are the same species, wherein the plurality of test peptide sequences comprises a ratio of 1:499 of the at least one hit peptide sequence to the at least 499 decoy peptide sequences and a top percentage of the plurality of test peptide sequences are predicted to be presented by the HLA protein expressed in cells by the machine learning HLA peptide presentation prediction model.

In some embodiments, the machine learning HLA peptide presentation prediction model has a positive predictive value (PPV) of at least 0.1 when amino acid information of a plurality of test peptide sequences are processed to generate a plurality of test binding predictions, each test binding prediction indicative of a likelihood that the one or more proteins encoded by a class II HLA allele of a cell of the subject binds to a given test peptide sequence of the plurality of test peptide sequences, wherein the plurality of test peptide sequences comprises at least 20 test peptide sequences comprising (i) at least one hit peptide sequence identified by mass spectrometry to be presented by an HLA protein expressed in cells and (ii) at least 19 decoy peptide sequences contained within a protein comprising at least one peptide sequence identified by mass spectrometry to be presented by an HLA protein expressed in cells, such as a single HLA protein expressed in cells (e.g., mono-allelic cells), wherein the plurality of test peptide sequences comprises a ratio of 1:19 of the at least one hit peptide sequence to the at least 19 decoy peptide sequences and a top percentage of the plurality of test peptide sequences are predicted to bind to the HLA protein expressed in cells by the machine learning HLA peptide presentation prediction model.

In some embodiments, no amino acid sequence overlap exist among the at least one hit peptide sequence and the decoy peptide sequences.

In some embodiments, the machine learning HLA peptide presentation prediction model has a positive predictive value (PPV) of at least 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98 or 0.99.

In some embodiments, the top percentage is a top 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, one engager of the bi- or trispecific engagers binds to an MHC on the target cell that is in complex with a cancer specific antigen peptide on a cancer cell. In some embodiments, the MHC protein encoded by an HLA wherein the HLA is a class I HLA (human leukocyte antigen (HLA)-A, -B, -C). In some embodiments, the MHC protein of the complex is a protein encoded by an HLA wherein the HLA is a class II HLA (HLA-DP, -DQ, or -DR). In some embodiments, the MHC protein in the MHC complex is encoded by HLA-A, -B, -C, -DP, -DQ, or -DR. In some embodiments, the MHC protein encoded by the HLA comprises an HLA class II protein selected from the group consisting of: HLA-DPB1*01:01/HLA-DPA1*01:03, HLA-DPB1*02:01/HLA-DPA1*01:03, HLA-DPB1*03:01/HLA-DPA1*01:03, HLA-DPB1*04:01/HLA-DPA1*01:03, HLA-DPB1*04:02/HLA-DPA1*01:03, HLA-DPB1*06:01/HLA-DPA1*01:03, HLA-DQB1*02:01/HLA-DQA1*05:01, HLA-DQB1*02:02/HLA-DQA1*02:01, HLA-DQB1*06:02/HLA-DQA1*01:02, HLA-DQB1*06:04/HLA-DQA1*01:02, HLA-DRB1*01:01, HLA-DRB1*01:02, HLA-DRB1*03:01, HLA-DRB1*03:02, HLA-DRB1*04:01, HLA-DRB1*04:02, HLA-DRB1*04:03, HLA-DRB1*04:04, HLA-DRB1*04:05, HLA-DRB1*04:07, HLA-DRB1*07:01, HLA-DRB1*08:01, HLA-DRB1*08:02, HLA-DRB1*08:03, HLA-DRB1*08:04, HLA-DRB1*09:01, HLA-DRB1*10:01, HLA-DRB1*11:01, HLA-DRB1*11:02, HLA-DRB1*11:04, HLA-DRB1*12:01, HLA-DRB1*12:02, HLA-DRB1*13:01, HLA-DRB1*13:02, HLA-DRB1*13:03, HLA-DRB1*14:01, HLA-DRB1*15:01, HLA-DRB1*15:02, HLA-DRB1*15:03, HLA-DRB1*16:01, HLA-DRB3*01:01, HLA-DRB3*02:02, HLA-DRB3*03:01, HLA-DRB4*01:01, HLA-DRB5*01:01.

In some embodiments, the HLA-DR is paired with paired with DRA*01:01.

In some embodiments, the MHC protein encoded by the HLA is a HLA class II protein selected from the group consisting of: DPA*01:03/DPB*04:01, DRB1*01:01, DRB1*01:02, DRB1*03:01, DRB1*04:01, DRB1*04:02, DRB1*04:04, DRB1*04:05, DRB1*07:01, DRB1*08:01, DRB1*08:02, DRB1*08:03, DRB1*09:01, DRB1*11:01, DRB1*11:02, DRB1*11:04, DRB1*12:01, DRB1*13:01, DRB1*13:02, DRB1*13:03, DRB1*14:01, DRB1*15:01, DRB1*15:02, DRB1*15:03, DRB1*16:02, DRB3*01:01, DRB3*02:01, DRB3*02:02, DRB3*03:01, DRB4*01:01, DRB4*01:03 and DRB5*01:01.

In some embodiments, the HLA-DR protein comprises a DRA*01:01 in the dimer.

In some embodiments, the MHC protein encoded by the HLA comprises an HLA-DP protein selected from the group consisting of: DPB1*01:01, DPB1*02:01, DPB1*02:02, DPB1*03:01, DPB1*04:01, DPB1*04:02, DPB1*05:01, DPB1*06:01, DPB1*11:01, DPB1*13:01, DPB1*17:01.

In some embodiments, the HLA-DP protein is paired with DPA1*01:03.

In some embodiments, the MHC protein encoded by the HLA comprises an HLA-DQ protein complex selected from the group consisting of: A1*01:01+B1*05:01, A1*01:02+B1*06:02, A1*01:02+B1*06:04, A1*01:03+B1*06:03, A1*02:01+B1*02:02, A1*02:01+B1*03:03, A1*03:01+B1*03:02, A1*03:03+B1*03:01, A1*05:01+B1*02:01 and A1*05:05+B1*03:01.

Sequences of class I and class II HLA alleles can be found in the IPD-IMGT/HLA Database. In some embodiments, the HLA allele is selected so as to correspond to a genotype of interest. In some embodiments, the HLA allele is a mutated HLA allele, which can be non-naturally occurring allele or a naturally occurring allele in an afflicted patient. The methods disclosed herein have the further advantage of identifying HLA binding peptides for HLA alleles associated with various disorders as well as alleles which are present at low frequency. For example, in some embodiments, method the HLA allele is present at a frequency of less than 1% within a population, such as within the Caucasian population.

In some embodiments, provided herein is a composition comprising a recombinant polynucleic acid that comprises a sequence encoding one or more polypeptides, e.g. a first polypeptide, and a second polypeptide. In some instances, throughout the description, the terms “first polypeptide” and “first polypeptide chain” may be used interchangeably; and the terms “second polypeptide” and “second polypeptide chain” may be used interchangeably.

In some embodiments, a polypeptide described herein comprises a first binding domain, a second binding domain operatively linked to the first domain, for example, the first domain is fused to the second domain. For example, the first binding domain and the second binding domain are operatively linked if C-terminus of the first domain is fused to N-terminus of the second domain, or alternatively, N-terminus of the first domain is fused to C-terminus of the second domain. For example, the first binding domain and the second binding domain are operatively linked if the C-terminus of the first domain is connected to the N-terminus of the second domain via a linker, e.g. a peptide linker or a chemical linker, or alternatively, N-terminus of the first domain is connected to the C-terminus of the second domain via a linker, e.g., a peptide linker. In some embodiments, the first polypeptide chain is configured to oligomerize with a second polypeptide chain. A second polypeptide may comprise one or more domains, that are operably linked to one another as described above, wherein the first binding domain specifically interacts with an antigen peptide in the context of MHC complex on a target cell, and wherein the second binding domain specifically interacts with a first extracellular protein of a T cell.

In one embodiment, an engager comprises two domains placed on two different polypeptide chains is assembly of the functional engager is achieved once the two polypeptide chains are structurally aligned in proper configuration following dimerization. Dimerization is achieved by one or more dimerization domains present on each polypeptide. In some embodiments, dimerization is improved by engineering the polypeptide: introducing one or mutations in the first or the second polypeptide or both.

In some embodiments, the MHC engager comprises a binder that binds an MHC-peptide complex, wherein the peptide is being presented by the MHC. The MHC engager comprises, at least variable domains of a T cell receptor (TCR), for example, a T cell alpha variable domain (TRAV) and a T cell beta variable domain (TRBV). In one embodiment, the MHC engager comprises alpha and beta T cell receptor variable domains (TRAV and TRBV) wherein the TRAV domain and TRBV domain occur in separate polypeptides, which upon dimerization of the polypeptides result in a functional MHC engager. For example TRAV constitute a domain on the first polypeptide, and TRBV constitute a domain on the second polypeptide. In some embodiments, the TRAV domain comprises a human TRAV domain. In some embodiments, the TRBV domain comprises a human TRBV domain.

In some embodiments, the TRAV or the TRBV domains comprise one or more mutations with respect to a human wild-type TRAV or the TRBV domains respectively.

Accordingly, provided herein is a multispecific engager, comprising a first polypeptide and a second polypeptide, wherein the first polypeptide comprises an MHC-engager domain, e.g., TRAV domain and wherein the second polypeptide comprises an MHC-engager domain, e.g., a TRBV domain and a T cell engager. In one embodiment, a first polypeptide comprises a TRAV domain, and a multimerization domain, and the second polypeptide comprises a TRBV domain and a multimerization domain, wherein the TRAV and the TRBV domain align or juxtapose upon dimerization of the first polypeptide and the second polypeptide, thereby forming the MHC-engager.

The multimerization domain of the first polypeptide and multimerization domain of the second polypeptide dimerize with each other post-translationally, and may comprise one or more disulfide bridges.

In some embodiments, the multimerization domains may comprise a TCR alpha contact domain (TRAC) one the first polypeptide, and TCR beta constant domain (TRBC) on the second polypeptide. Various other exemplary dimerization and oligomerization domains are disclosed herein.

In some embodiments, the TRAC domain and the TRBC domain comprise human TRAC and human TRBC domain constant domains.

In some embodiments, the TRAC domain or the TRBC domain comprises one or more mutations with respect to wild-type human TRAC and TRBC domains.

In one embodiment, provided herein is a recombinant polypeptide construct, comprising two or more polypeptides, comprising a first polypeptide comprising a binding domain, and a dimerization domain; a second polypeptide comprising a binding domain and a dimerization domain; for example, a first polypeptide comprising a TRAV domain and a TRAC domain; a second polypeptide comprising a TRBV domain and a TRBC domain, and wherein the TRBV domain or the TRBC domain may additionally comprise a T cell engager domain.

In an alternative embodiment, provided herein is a recombinant polypeptide construct, comprising two or more polypeptides, comprising a first polypeptide comprising a binding domain, and a dimerization domain; a second polypeptide comprising a binding domain and a dimerization domain; for example, a first polypeptide comprising a TRAV domain and a TRAC domain; a second polypeptide comprising a TRBV domain and a TRBC domain, and wherein the TRAV domain or the TRAC domain may additionally comprise a T cell engager domain.

Provided herein is a bispecific recombinant construct, comprising a first polypeptide and a second polypeptide, comprising a first engager comprising an MHC binder domain, comprising a first binding domain on the first polypeptide and a second binding domain on the second polypeptide; and a second engager, comprising a T cell engager present on the second polypeptide.

In some embodiments, for example, the cancer specific antigen peptide for a target cancer cell can be one or more of the mutated/cancer antigens from mutated proteins e.g., KRAS, GATA3, MAGE1, PRAME, NYESO-1, ABL1, EGFR, ERBB3, FGR3, BCR, ALK, p53, CD2, CD19, CD20, CD22, CD27, CD33, CD37, CD38, CD40, CD44, CD47, CD52, CD56, CD70, 30 CD79, CD137, 4-IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin av, Integrin av3, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, or VEGFR-3, among others. In some embodiments, one of the aforementioned targets may be contemplated in generating a target cell specific binder or soluble TCR, having an engager that comprises the TRAV/TRBV domains, that specifically binds to an antigenic peptide of one of the aforementioned exemplary gene products, presented by a suitable MHC protein, and wherein the binder comprises a second engager, that binds to a cell surface component of a cell e.g., a T cell.

a. MHC:Peptide (p:MHC) Complexes

In some embodiments, an MHC-peptide complex engager is designed based on the identification of the cancer specific antigen that is to be targeted. In some embodiments, for example, the cancer specific antigen peptide is a KRAS antigen. In some embodiments, the cancer specific KRAS antigen comprises a mutation with respect to KRAS antigens that are present in non-cancerous healthy and normal cells and tissues. In some embodiments, (i) the activating oncogene mutation is a KRAS mutation; (ii) the KRAS mutation is a G12 mutation, optionally wherein the G12 KRAS mutation is selected from a G12D, G12V, G12S, G12C, G12A, and a G12R KRAS mutation; (iii) the KRAS mutation is a G13 mutation, optionally wherein the G13 KRAS mutation is a G13D KRAS mutation; and/or (iv) the activating oncogene mutation is a H-RAS or N-RAS mutation. The KRAS antigens or epitopes, and the corresponding MHC molecules encoded by an HLA allele that can bind and present the epitope to a T cell may be obtained from prior investigations in the field. For example, a mutant RAS peptide sequence may be selected from the group consisting of: DTAGHEEY, TAGHEEYSAM, DILDTAGHE, DILDTAGH, ILDTAGHEE, ILDTAGHE, DILDTAGHEEY, DTAGHEEYS, LLDILDTAGH, DILDTAGRE, DILDTAGR, ILDTAGREE, ILDTAGRE, CLLDILDTAGR, TAGREEYSAM, REEYSAMRD, DTAGKEEYSAM, CLLDILDTAGK, DTAGKEEY, LLDILDTAGK, ILDTAGKE, ILDTAGKEE, DTAGLEEY, ILDTAGLE, DILDTAGL, ILDTAGLEE, GLEEYSAMRDQY, LLDILDTAGLE, LDILDTAGL, DILDTAGLE, DILDTAGLEEY, AGVGKSAL, GAAGVGKSAL, AAGVGKSAL, CGVGKSAL, ACGVGKSAL, DGVGKSAL, ADGVGKSAL, DGVGKSALTI, GARGVGKSA, KLVVVGARGV, VVVGARGV, SGVGKSAL, VVVGASGVGK, GASGVGKSAL, VGVGKSAL, VVVGAGCVGK, KLVVVGAGC, GDVGKSAL, DVGKSALTI, VVVGAGDVGK, TAGKEEYSAM, DTAGHEEYSAM, TAGHEEYSA, DTAGREEYSAM, TAGKEEYSA, AAGVGKSA, AGCVGKSAL, AGDVGKSAL, AGKEEYSAMR, AGVGKSALTI, ARGVGKSAL, ASGVGKSA, ASGVGKSAL, AVGVGKSA, CVGKSALTI, DILDTAGK, DILDTAGREEY, DTAGHEEYSAMR, DTAGKEEYS, DTAGKEEYSAMR, DTAGLEEYS, DTAGLEEYSA, DTAGLEEYSAMR, DTAGREEYS, DTAGREEYSAMR, GAAGVGKSA, GACGVGKSA, GACGVGKSAL, GADGVGKS, GAGDVGKSA, GAGDVGKSAL, GASGVGKSA, GCVGKSAL, GCVGKSALTI, GHEEYSAM, GKEEYSAM, GLEEYSAMR, GREEYSAM, GREEYSAMR, HEEYSAMRD, KEEYSAMRD, KLVVVGASG, LDILDTAGR, LEEYSAMRD, LVVVGARGV, LVVVGASGV, REEYSAMRDQY, RGVGKSAL, TAGLEEYSA, TEYKLVVVGAA, VGAAGVGKSA, VGADGVGK, VGASGVGKSA, VGVGKSALTI, VVVGAAGV, VVVGAVGV, YKLVVVGAC, YKLVVVGAD, YKLVVVGAR, and DILDTAGKE; or (b) at least one polynucleotide encoding the at least one polypeptide.

In some embodiments, a mutant RAS peptide sequence may be selected from the group consisting of: KLVVVGADGV, KLVVVGACGV, KLVVVGAVGV, LVVVGADGV, LVVVGACGV, LVVVGAVGV; GADGVGKSAL, GACGVGKSAL, GAVGVGKSAL, GADGVGKSA, GACGVGKSA, GAVGVGKSA; and/or VVGADGVGK, VVGACGVGK, VVGAVGVGK, VVVGADGVGK, VVVGACGVGK, VVVGAVGVGK.

In some embodiments, a mutant RAS peptide sequence may be selected from the group consisting of LVVVGACGV, KLVVVGACGV, LVVVGADGV, KLVVVGADGV, LVVVGAVGV, KLVVVGAVGV, VVGACGVGK, VVVGACGVGK, VVGADGVGK, VVVGADGVGK, VVGAVGVGK, VVVGAVGVGK, VVGACGVGK, VVGADGVGK, VVVGADGVGK, VVGAVGVGK, and VVVGAVGVGK.

In some embodiments, a mutant RAS peptide sequence may be selected from the group consisting of VVGADGVGK, VVGACGVGK, VVGAVGVGK, VVVGADGVGK, VVVGACGVGK, VVVGAVGVGK.

In some embodiments, a mutant RAS peptide sequence may be selected from the group consisting of GADGVGKSAL, GACGVGKSAL, GAVGVGKSAL, GADGVGKSA, GACGVGKSA, or GAVGVGKSA.

In some embodiments, the mutant RAS peptide sequences comprises at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids.

In some embodiments, a mutant RAS peptide sequence binds to or is predicted to bind to a protein encoded by an HLA-A02:01 allele, an HLA-A03:01 allele, an HLA-A11:01 allele, and/or an HLA-C08:02 allele.

In some embodiments, a mutant RAS peptide sequence binds or is predicted to bind to a protein encoded by: an HLA-A02:01 allele and an HLA-A03:01 allele, an HLA-A11:01 allele, an HLA-A03:02 allele, an HLA-A30:01 allele, an HLA-A31:01 allele, an HLA-A33:01 allele, an HLA-A33:03 allele, an HLA-A68:01 allele, or an HLA-A74:01 allele; an HLA-A02:01 allele and an HLA-C08:02 allele; an HLA-A03:01 allele, an HLA-A11:01 allele, an HLA-A03:02 allele, an HLA-A30:01 allele, an HLA-A31:01 allele, an HLA-A33:01 allele, an HLA-A33:03 allele, an HLA-A68:01 allele, or an HLA-A74:01 allele and an HLA-C08:02 allele; or an HLA-A03:01 allele, an HLA-A11:01 allele, an HLA-A03:02 allele, an HLA-A30:01 allele, an HLA-A31:01 allele, an HLA-A33:01 allele, an HLA-A33:03 allele, an HLA-A68:01 allele, or an HLA-A74:01 allele and allele and an HLA-A03:01 allele, an HLA-A11:01 allele, an HLA-A03:02 allele, an HLA-A30:01 allele, an HLA-A31:01 allele, an HLA-A33:01 allele, an HLA-A33:03 allele, an HLA-A68:01 allele, or an HLA-A74:01 allele.

In some embodiments, the at least one polypeptide comprises at least one mutant RAS peptide sequence that binds to a protein encoded by an HLA allele with an affinity of less than 10 μM, less than 1 μM, less than 500 nM, less than 400 nM, less than 300 nM, less than 250 nM, less than 200 nM, less than 150 nM, less than 100 nM, or less than 50 nM.

In some embodiments the binding potential of shared antigens such as KRAS mutations is predicted by a MHC-peptide binding prediction analysis software that ranks the peptide:MVHC pairs according to predicted binding affinity, rank one having the highest affinity.

TABLE 1
Peptide Sequences Comprising RAS Q61H Mutation, Corresponding HLA Allele,
and Rank of Binding Potential. Ranking of binding potential is a measure
of MHC:peptide binding efficiency converted to an arbitrary integer
figures, with “1” being the most potent and strongest binding and
gradually losing with increasing numerical value. In some embodiments,
a peptide sequence provided in Table 1 binds to or is predicted to
bind to a protein encoded by an HLA allele, which allele is provided
in a corresponding column in Table 1 next to the peptide sequence.

| Peptide | Allele | Rank of Binding Potential |
|---|---|---|
| ILDTAGHEEY | HLA-A36:01 | 1 |
| ILDTAGHEEY | HLA-A01:01 | 2 |
| DTAGHEEYSAM | HLA-A26:01 | 3 |
| DTAGHEEYSAM | HLA-A25:01 | 4 |
| GHEEYSAM | HLA-B15:09 | 4 |
| DTAGHEEY | HLA-A26:01 | 5 |
| ILDTAGHEE | HLA-C08:02 | 5 |
| AGHEEYSAM | HLA-C01:02 | 6 |
| AGHEEYSAM | HLA-B46:01 | 6 |
| DTAGHEEY | HLA-A25:01 | 6 |
| DTAGHEEY | HLA-A01:01 | 6 |
| DTAGHEEY | HLA-B18:01 | 7 |
| DTAGHEEY | HLA-A36:01 | 7 |
| ILDTAGHEE | HLA-C05:01 | 7 |
| ILDTAGHEE | HLA-A02:07 | 7 |
| ILDTAGHEEY | HLA-A29:02 | 7 |
| ILDTAGHEEY | HLA-C08:02 | 7 |
| HEEYSAMRD | HLA-B49:01 | 8 |
| TAGHEEYSA | HLA-B35:03 | 8 |
| DTAGHEEYS | HLA-A68:02 | 9 |
| DTAGHEEYSAMR | HLA-A68:01 | 9 |
| GHEEYSAM | HLA-B39:01 | 9 |
| ILDTAGHEE | HLA-A01:01 | 9 |
| LDTAGHEEY | HLA-B53:01 | 9 |
| HEEYSAMRD | HLA-B41:01 | 10 |
| ILDTAGHEE | HLA-A36:01 | 10 |
| DTAGHEEY | HLA-B58:01 | 11 |
| LLDILDTAGH | HLA-A01:01 | 12 |
| TAGHEEYSAM | HLA-B35:03 | 12 |
| LDTAGHEEY | HLA-B35:01 | 13 |
| DILDTAGHE | HLA-A26:01 | 14 |
| DTAGHEEY | HLA-C12:03 | 14 |
| ILDTAGHEEY | HLA-C05:01 | 14 |
| AGHEEYSAM | HLA-A30:02 | 15 |
| DILDTAGHEEY | HLA-A25:01 | 15 |
| DTAGHEEY | HLA-C02:02 | 15 |
| ILDTAGHEE | HLA-C04:01 | 15 |
| DILDTAGH | HLA-A26:01 | 16 |
| ILDTAGHEE | HLA-A02:01 | 16 |
| LDTAGHEEY | HLA-A29:02 | 16 |
| ILDTAGHE | HLA-A01:01 | 17 |
| LDTAGHEEY | HLA-B18:01 | 17 |
| AGHEEYSAM | HLA-C14:03 | 18 |
| DILDTAGHEEY | HLA-A29:02 | 18 |
| DTAGHEEYS | HLA-A26:01 | 18 |
| ILDTAGHEEY | HLA-B15:01 | 18 |
| DTAGHEEYSA | HLA-A68:02 | 19 |
| ILDTAGHE | HLA-C05:01 | 19 |
| ILDTAGHEEY | HLA-A02:07 | 19 |
| ILDTAGHEEY | HLA-A30:02 | 19 |
| LDTAGHEEY | HLA-A36:01 | 19 |
| AGHEEYSAM | HLA-C14:02 | 20 |
| AGHEEYSAM | HLA-B15:03 | 20 |
| LLDILDTAGH | HLA-A02:07 | 20 |

In some embodiments, a peptide comprising a RAS Q61R mutation comprises a sequence of TCLLDILDTAGREEYSAMRDQYM. In some embodiments, a peptide comprising a RAS Q61R mutation comprises a sequence provided in Table 2.

TABLE 2
Peptide Sequences Comprising RAS Q61R Mutation, Corresponding HLA Allele,
and Rank of Binding Potential obtained from Neon MHC program.

| Peptide | Allele | Rank of Binding Potential |
|---|---|---|
| ILDTAGREEY | HLA-A36:01 | 1 |
| ILDTAGREEY | HLA-A01:01 | 2 |
| DTAGREEYSAM | HLA-A26:01 | 3 |
| DILDTAGR | HLA-A33:03 | 4 |
| DILDTAGR | HLA-A68:01 | 5 |
| DTAGREEY | HLA-A26:01 | 6 |
| DTAGREEYSAM | HLA-A25:01 | 6 |
| CLLDILDTAGR | HLA-A74:01 | 7 |
| DTAGREEY | HLA-A01:01 | 7 |
| REEYSAMRD | HLA-B41:01 | 7 |
| GREEYSAMR | HLA-B27:05 | 8 |
| ILDTAGREE | HLA-C08:02 | 8 |
| ILDTAGREEY | HLA-A29:02 | 8 |
| REEYSAMRD | HLA-B49:01 | 8 |
| AGREEYSAM | HLA-B46:01 | 9 |
| DTAGREEY | HLA-B18:01 | 9 |
| DTAGREEY | HLA-A25:01 | 9 |
| DTAGREEY | HLA-A36:01 | 9 |
| DILDTAGR | HLA-A74:01 | 10 |
| DILDTAGRE | HLA-A26:01 | 10 |
| ILDTAGREE | HLA-C05:01 | 10 |
| DILDTAGR | HLA-A26:01 | 11 |
| GREEYSAM | HLA-B39:01 | 11 |
| AGREEYSAM | HLA-B15:03 | 12 |
| GREEYSAM | HLA-C07:02 | 12 |
| ILDTAGREE | HLA-A01:01 | 12 |
| TAGREEYSA | HLA-B35:03 | 12 |
| ILDTAGREEY | HLA-A30:02 | 13 |
| DTAGREEYS | HLA-A68:02 | 14 |
| ILDTAGRE | HLA-A01:01 | 14 |
| CLLDILDTAGR | HLA-A31:01 | 15 |
| DTAGREEYSAMR | HLA-A68:01 | 15 |
| LLDILDTAGR | HLA-A01:01 | 15 |
| DTAGREEY | HLA-B58:01 | 16 |
| ILDTAGREEY | HLA-C08:02 | 16 |
| DILDTAGR | HLA-A31:01 | 17 |
| ILDTAGREE | HLA-C04:01 | 17 |
| ILDTAGREEY | HLA-A32:01 | 17 |
| LLDILDTAGR | HLA-A74:01 | 17 |
| TAGREEYSAM | HLA-B35:03 | 17 |
| DILDTAGREEY | HLA-A32:01 | 18 |
| ILDTAGRE | HLA-C05:01 | 18 |
| ILDTAGREE | HLA-A02:07 | 18 |
| REEYSAMRD | HLA-B40:01 | 18 |
| AGREEYSAM | HLA-B15:01 | 19 |
| AGREEYSAMR | HLA-A31:01 | 19 |
| ILDTAGRE | HLA-A36:01 | 19 |
| LDILDTAGR | HLA-A68:01 | 19 |
| LDTAGREEY | HLA-A29:02 | 19 |
| LDTAGREEY | HLA-B35:01 | 19 |
| REEYSAMRD | HLA-B45:01 | 19 |
| REEYSAMRDQY | HLA-A36:01 | 19 |
| DTAGREEY | HLA-C02:02 | 20 |

In some embodiments, a peptide comprising a RAS Q61K mutation comprises a sequence of TCLLDILDTAGKEEYSAMRDQYM. In some embodiments, a peptide comprising a RAS Q61K mutation comprises a sequence provided in Table 3. In some embodiments, a peptide sequence provided in Table 3 binds to or is predicted to bind to a protein encoded by an HLA allele, which allele is provided in a corresponding column in Table 3 next to the peptide sequence.

TABLE 3
Peptide Sequences Comprising RAS Q61K Mutation, Corresponding
HLA Allele, and Rank of Binding Potential

| Peptide | Allele | Rank of Binding Potential |
|---|---|---|
| ILDTAGKEEY | HLA-A36:01 | 1 |
| ILDTAGKEEY | HLA-A01:01 | 2 |
| DTAGKEEYSAM | HLA-A26:01 | 3 |
| CLLDILDTAGK | HLA-A03:01 | 4 |
| DTAGKEEY | HLA-A01:01 | 5 |
| DTAGKEEY | HLA-A26:01 | 5 |
| DTAGKEEYSAM | HLA-A25:01 | 5 |
| AGKEEYSAM | HLA-B46:01 | 6 |
| DILDTAGKE | HLA-A26:01 | 7 |
| KEEYSAMRD | HLA-B41:01 | 7 |
| DTAGKEEY | HLA-B18:01 | 8 |
| GKEEYSAM | HLA-B15:03 | 8 |
| ILDTAGKEE | HLA-C08:02 | 8 |
| ILDTAGKEEY | HLA-A29:02 | 8 |
| DTAGKEEYS | HLA-A68:02 | 9 |
| LDTAGKEEY | HLA-B53:01 | 9 |
| TAGKEEYSA | HLA-B35:03 | 9 |
| DILDTAGK | HLA-A68:01 | 10 |
| DTAGKEEY | HLA-A36:01 | 10 |
| KEEYSAMRD | HLA-B49:01 | 10 |
| LDTAGKEEY | HLA-C07:01 | 10 |
| DTAGKEEYSAMR | HLA-A68:01 | 11 |
| ILDTAGKEE | HLA-C05:01 | 11 |
| ILDTAGKEEY | HLA-C08:02 | 11 |
| LLDILDTAGK | HLA-A01:01 | 12 |
| AGKEEYSAM | HLA-A30:02 | 13 |
| DTAGKEEY | HLA-A25:01 | 13 |
| DTAGKEEYS | HLA-A26:01 | 13 |
| ILDTAGKE | HLA-C05:01 | 13 |
| LDTAGKEEY | HLA-B35:01 | 13 |
| AGKEEYSAMR | HLA-A31:01 | 14 |
| DILDTAGK | HLA-A33:03 | 14 |
| ILDTAGKE | HLA-A01:01 | 14 |
| ILDTAGKEE | HLA-A01:01 | 14 |
| ILDTAGKEE | HLA-A02:07 | 14 |
| TAGKEEYSAM | HLA-B35:03 | 14 |
| AGKEEYSAM | HLA-B15:01 | 15 |
| ILDTAGKEEY | HLA-A30:02 | 15 |
| LDTAGKEEY | HLA-B46:01 | 15 |
| DTAGKEEY | HLA-B58:01 | 16 |
| ILDTAGKEEY | HLA-C05:01 | 17 |
| AGKEEYSAM | HLA-A30:01 | 18 |
| AGKEEYSAM | HLA-B15:03 | 18 |
| DTAGKEEY | HLA-C02:02 | 18 |
| LDTAGKEEY | HLA-A29:02 | 18 |

In some embodiments, a peptide comprising a RAS Q61L mutation comprises a sequence of TCLLDILDTAGLEEYSAMRDQYM. In some embodiments, a peptide comprising a RAS Q61L mutation comprises a sequence provided in Table 4. In some embodiments, a peptide sequence provided in Table 4 binds to or is predicted to bind to a protein encoded by an HLA allele, which allele is provided in a corresponding column in Table 4 next to the peptide sequence.

TABLE 4
Peptide Sequences Comprising RAS Q61L Mutation, Corresponding
HLA Allele, and Rank of Binding Potential

| Peptide | Allele | Rank of Binding Potential |
|---|---|---|
| ILDTAGLEEY | HLA-A36:01 | 1 |
| ILDTAGLEEY | HLA-A01:01 | 2 |
| LLDILDTAGL | HLA-A02:07 | 3 |
| GLEEYSAMRDQY | HLA-A36:01 | 4 |
| DTAGLEEY | HLA-A25:01 | 5 |
| DTAGLEEY | HLA-A26:01 | 5 |
| DTAGLEEYSAM | HLA-A26:01 | 5 |
| DTAGLEEY | HLA-A01:01 | 6 |
| ILDTAGLEE | HLA-C08:02 | 6 |
| ILDTAGLEE | HLA-A01:01 | 6 |
| CLLDILDTAGL | HLA-A02:04 | 7 |
| ILDTAGLEE | HLA-A36:01 | 7 |
| LLDILDTAGL | HLA-A01:01 | 7 |
| DILDTAGL | HLA-B14:02 | 8 |
| DILDTAGLEEY | HLA-A25:01 | 8 |
| DTAGLEEYS | HLA-A68:02 | 8 |
| DTAGLEEYSAM | HLA-A25:01 | 8 |
| GLEEYSAMR | HLA-A74:01 | 8 |
| ILDTAGLE | HLA-A01:01 | 8 |
| DILDTAGLEEY | HLA-A26:01 | 9 |
| DTAGLEEY | HLA-A36:01 | 9 |
| ILDTAGLEEY | HLA-A29:02 | 9 |
| DILDTAGL | HLA-B08:01 | 10 |
| DTAGLEEY | HLA-B18:01 | 10 |
| ILDTAGLEE | HLA-A02:07 | 10 |
| LDTAGLEEY | HLA-B35:01 | 10 |
| CLLDILDTAGL | HLA-A02:01 | 11 |
| DTAGLEEY | HLA-C02:02 | 11 |
| ILDTAGLEE | HLA-C05:01 | 11 |
| ILDTAGLEEY | HLA-C08:02 | 11 |
| ILDTAGLEEY | HLA-A02:07 | 11 |
| LLDILDTAGL | HLA-C08:02 | 11 |
| DILDTAGL | HLA-A26:01 | 12 |
| LDTAGLEEY | HLA-B53:01 | 12 |
| DTAGLEEY | HLA-C03:02 | 13 |
| DTAGLEEY | HLA-B58:01 | 13 |
| ILDTAGLEEY | HLA-A30:02 | 13 |
| LLDILDTAGL | HLA-C05:01 | 13 |
| LLDILDTAGL | HLA-C04:01 | 13 |
| DTAGLEEYSAMR | HLA-A68:01 | 14 |
| ILDTAGLE | HLA-A36:01 | 15 |
| LLDILDTAGL | HLA-A02:01 | 15 |
| AGLEEYSAM | HLA-B15:03 | 16 |
| DTAGLEEYSA | HLA-A68:02 | 16 |
| GLEEYSAMRDQY | HLA-A01:01 | 16 |
| ILDTAGLE | HLA-C04:01 | 16 |
| ILDTAGLEEY | HLA-B15:01 | 16 |
| LDILDTAGL | HLA-B37:01 | 16 |
| AGLEEYSAM | HLA-A30:02 | 17 |
| AGLEEYSAM | HLA-B48:01 | 17 |
| AGLEEYSAMR | HLA-A31:01 | 17 |
| ILDTAGLEE | HLA-C04:01 | 17 |
| LDTAGLEEY | HLA-C03:02 | 17 |
| AGLEEYSAM | HLA-C14:02 | 18 |
| GLEEYSAMR | HLA-A31:01 | 18 |
| LEEYSAMRD | HLA-B41:01 | 18 |
| LLDILDTAGLE | HLA-A01:01 | 18 |
| AGLEEYSAM | HLA-C14:03 | 19 |
| LDILDTAGL | HLA-B40:02 | 19 |
| LDTAGLEEY | HLA-A29:02 | 19 |
| DILDTAGLE | HLA-A26:01 | 20 |
| DTAGLEEY | HLA-B15:01 | 20 |
| ILDTAGLEEY | HLA-A02:01 | 20 |
| LDTAGLEEY | HLA-A36:01 | 20 |
| LDTAGLEEY | HLA-B46:01 | 20 |
| DTAGLEEY | HLA-A68:02 | 21 |
| DTAGLEEY | HLA-C12:03 | 21 |
| ILDTAGLE | HLA-C05:01 | 21 |
| LDTAGLEEY | HLA-B18:01 | 21 |
| LEEYSAMRD | HLA-B49:01 | 21 |
| TAGLEEYSA | HLA-B54:01 | 21 |
| DILDTAGLEEY | HLA-A29:02 | 22 |
| GLEEYSAM | HLA-C05:01 | 22 |

In some embodiments, the variable domain sequences may be derived from TCR sequences isolated and characterized in earlier investigations. A TCR contains variable complementarity determining regions (CDRs), as well as framework regions (FRs) and a constant region. The amino acid sequence of the third complementarity-determining region (CDR3) loops of the α and β chain variable domains largely determines the sequence diversity of α/β T cells arising from recombination between variable (Vβ), diversity (Dβ), and joining (Jβ) gene segments in the β chain locus, and between analogous Vα and Jα gene segments in the α chain locus, respectively. The existence of multiple such gene segments in the TCR α and β chain loci allows for a large number of distinct CDR3 sequences to be encoded. Independent addition and deletion of nucleotides at the Vβ-Dβ, Dβ-Jβ, and Vα-Jα Junctions during the process of TCR gene rearrangement further increases CDR3 sequence diversity. In this respect, immunocompetence is reflected in the diversity of TCRs.

TABLE 5
Some exemplary RAS-TCR sequences that were previously identified are provided
below. An exemplary sTCR design for MHC-RAS complex may be designed using
the relevant variable regions from the following TCRs

| | SEQ | |
|---|---|---|
| | ID | |
| Name | NO | Sequence |
| RAS TCR-1 | 1 | SIFNT |
| alpha chain | | |
| CDR1 | | |
| RAS TCR-1 | 2 | LYKAGEL |
| alpha chain | | |
| CDR2 | | |
| RAS TCR-1 | 3 | CAGRNFGNEKLTF |
| alpha chain | | |
| CDR3 | | |
| RAS TCR-1 | 4 | DFQATT |
| beta chain | | |
| CDR1 | | |
| RAS TCR-1 | 5 | SNEGSKA |
| beta chain | | |
| CDR2 | | |
| RAS TCR-1 | 6 | SARDRGLVSLPSVEAFF |
| beta chain | | |
| CDR3 | | |
| RAS TCR-1 | 7 | ATGCTCCTTGAACATTTATTAATAATCTTGTGGATGCAGCTGACA |
| alpha chain | | TGGGTCAGTGGTCAACAGCTGAATCAGAGTCCTCAATCTATGTTT |
| variable | | ATCCAGGAAGGAGAAGATGTCTCCATGAACTGCACTTCTTCAAGC |
| domain | | ATATTTAACACCTGGCTATGGTACAAGCAGGACCCTGGGGAAGG |
| | | TCCTGTCCTCTTGATAGCCTTATATAAGGCTGGTGAATTGACCTC |
| | | AAATGGAAGACTGACTGCTCAGTTTGGTATAACCAGAAAGGACA |
| | | GCTTCCTGAATATCTCAGCATCCATACCTAGTGATGTAGGCATCT |
| | | ACTTCTGTGCTGGGAGAAACTTTGGAAATGAGAAATTAACCTTTG |
| | | GGACTGGAACAAGACTCACCATCATACCC |
| | 8 | ATGCTCCTTGAACATTTATTAATAATCTTGTGGATGCAGCTGACA |
| | | TGGGTCAGTGGTCAACAGCTGAATCAGAGTCCTCAATCTATGTTT |
| | | ATCCAGGAAGGAGAAGATGTCTCCATGAACTGCACTTCTTCAAGC |
| | | ATATTTAACACCTGGCTATGGTACAAGCAGGACCCTGGGGAAGG |
| | | TCCTGTCCTCTTGATAGCCTTATATAAGGCTGGTGAATTGACCTC |
| | | AAATGGAAGACTGACTGCTCAGTTTGGTATAACCAGAAAGGACA |
| | | GCTTCCTGAATATCTCAGCATCCATACCTAGTGATGTAGGCATCT |
| | | ACTTCTGTGCTGGGAGAAACTTTGGAAATGAGAAATTAACCTTTG |
| | | GGACTGGAACAAGACTCACCATCATACCC |
| | 9 | MLLEHLLIILWMQLTWVSGQQLNQSPQSMFIQEGEDVSMNCTSSS |
| | | IFNTWLWYKQDPGEGPVLLIALYKAGELTSNGRLTAQFGITRKDS |
| | | FLNISASIPSDVGIYFCAGRNFGNEKLTFGTGTRLTIIP |
| RAS TCR-1 | 10 | ATGCTGCTGCTTCTGCTGCTTCTGGGGCCAGGCTCCGGGCTTGGT |
| beta chain | | GCTGTCGTCTCTCAACATCCGAGCAGGGTTATCTGTAAGAGTGGA |
| variable | | ACCTCTGTGAAGATCGAGTGCCGTTCCCTGGACTTTCAGGCCACA |
| domain | | ACTATGTTTTGGTATCGTCAGTTCCCGAAACAGAGTCTCATGCTG |
| | | ATGGCAACTTCCAATGAGGGCTCCAAGGCCACATACGAGCAAGG |
| | | CGTCGAGAAGGACAAGTTTCTCATCAACCATGCAAGCCTGACCTT |
| | | GTCCACTCTGACAGTGACCAGTGCCCATCCTGAAGACAGCAGCTT |
| | | CTACATCTGCAGTGCTCGCGACAGGGGGCTTGTATCGTTGCCGTC |
| | | GGTAGAAGCTTTCTTTGGACAAGGCACCAGACTCACAGTTGTACT |
| | | G |
| | 11 | ATGCTGCTGCTTCTGCTGCTTCTGGGGCCAGGCTCCGGGCTTGGT |
| | | GCTGTCGTCTCTCAACATCCGAGCAGGGTTATCTGTAAGAGTGGA |
| | | ACCTCTGTGAAGATCGAGTGCCGTTCCCTGGACTTTCAGGCCACA |
| | | ACTATGTTTTGGTATCGTCAGTTCCCGAAACAGAGTCTCATGCTG |
| | | ATGGCAACTTCCAATGAGGGCTCCAAGGCCACATACGAGCAAGG |
| | | CGTCGAGAAGGACAAGTTTCTCATCAACCATGCAAGCCTGACCTT |
| | | GTCCACTCTGACAGTGACCAGTGCCCATCCTGAAGACAGCAGCTT |
| | | CTACATCTGCAGTGCTCGCGACAGGGGGCTTGTATCGTTGCCGTC |
| | | GGTAGAAGCTTTCTTTGGACAAGGCACCAGACTCACAGTTGTACT |
| | | G |
| | 12 | MLLLLLLLGPGSGLGAVVSQHPSRVICKSGTSVKIECRSLDFQAT |
| | | TMFWYRQFPKQSLMLMATSNEGSKATYEQGVEKDKFLINHASLTL |
| | | STLTVTSAHPEDSSFYICSARDRGLVSLPSVEAFFGQGTRLTVV |
| RAS TCR-1 | 13 | MLLEHLLIILWMQLTWVSGQQLNQSPQSMFIQEGEDVSMNCTSSSIF |
| alpha chain | | NTWLWYKQDPGEGPVLLIALYKAGELTSNGRLTAQFGITRKDSFLNI |
| | | SASIPSDVGIYFCAGRNFGNEKLTFGTGTRLTIIPDIQNPDPAVYQL |
| | | RDSKSSDKSVCLFTDFDSQTNVSQSKDSDVYITDKTVLDMRSMDFKS |
| | | NSAVAWSNKSDFACANAFNNSIIPEDTFFPSPESSCDVKLVEKSFET |
| | | DTNLNFQNLSVIGFRILLLKVAGENLLMTLRLWSS |
| RAS TCR-1 | 14 | MLLLLLLLGPGSGLGAVVSQHPSRVICKSGTSVKIECRSLDFQATTM |
| beta chain | | FWYRQFPKQSLMLMATSNEGSKATYEQGVEKDKFLINHASLTLSTLT |
| | | VTSAHPEDSSFYICSARDRGLVSLPSVEAFFGQGTRLTVVLEDLNKV |
| | | FPPEVAVFEPSEAEISHTQKATLVCLATGFFPDHVELSWWVNGKE |
| | | VHSGVSTDPQPLKEQPALNDSRYCLSSRLRVSATFWQNPRNHFRCQ |
| | | VQFYGLSENDEWTQDRAKPVTQIVSAEAWGRADCGFTSVSYQQGV |
| | | LSATILYEILLGKATLYAVLVSALVLMAMVKRKDF |
| RAS TCR- | 15 | KLVVVGACGV |
| 1/2 peptide | | |
| RAS TCR-2 | 16 | VSGNPY |
| alpha chain | | |
| CDR1 | | |
| RAS TCR-2 | 17 | YITGDNLV |
| alpha chain | | |
| CDR2 | | |
| RAS TCR-2 | 18 | CAVRDQSGANNLFF |
| alpha chain | | |
| CDR3 | | |
| RAS TCR-2 | 19 | SEHNR |
| beta chain | | |
| CDR1 | | |
| RAS TCR-2 | 20 | FQNEAQ |
| beta chain | | |
| CDR2 | | |
| Ras TCR-2 | 21 | ASYLSGSIYNEQFF |
| beta chain | | |
| CDR3 | | |
| RAS TCR-2 | 22 | ATGGCCTCTGCACCCATCTCGATGCTTGCGATGCTCTTCACATTG |
| alpha chain | | AGTGGGCTGAGAGCTCAGTCAGTGGCTCAGCCGGAAGATCAGGT |
| variable | | CAACGTTGCTGAAGGGAATCCTCTGACTGTGAAATGCACCTATTC |
| domain | | AGTCTCTGGAAACCCTTATCTTTTTTGGTATGTTCAATACCCCAAC |
| | | CGAGGCCTCCAGTTCCTTCTGAAATACATCACAGGGGATAACCTG |
| | | GTTAAAGGCAGCTATGGCTTTGAAGCTGAATTTAACAAGAGCCA |
| | | AACCTCCTTCCACCTGAAGAAACCATCTGCCCTTGTGAGCGACTC |
| | | CGCTTTGTACTTCTGTGCTGTGAGAGACCAAAGTGGGGCAAACAA |
| | | CCTCTTCTTTGGGACTGGAACGAGACTCACCGTTATTCCC |
| | 23 | ATGGCTTCTGCGCCTATATCAATGCTTGCCATGCTGTTTACACTGT |
| | | CCGGTCTGAGGGCTCAAAGCGTGGCCCAACCTGAGGATCAGGTG |
| | | AATGTAGCGGAGGGCAATCCGTTGACAGTTAAGTGTACATACTCC |
| | | GTATCAGGCAATCCGTACTTGTTTTGGTATGTGCAGTACCCCAAT |
| | | CGGGGGCTTCAATTCTTGCTGAAGTACATTACAGGCGATAATCTG |
| | | GTAAAAGGTAGTTATGGTTTTGAGGCCGAATTCAACAAATCACA |
| | | AACATCATTTCATCTTAAAAAGCCAAGCGCACTTGTCAGTGACTC |
| | | AGCGCTTTATTTCTGTGCAGTCAGAGACCAATCAGGGGCAAATAA |
| | | TCTGTTCTTTGGGACAGGGACTAGATTGACTGTTATACCC |
| | 24 | MASAPISMLAMLFTLSGLRAQSVAQPEDQVNVAEGNPLTVKCTYSV |
| | | SGNPYLFWYVQYPNRGLQFLLKYITGDNLVKGSYGFEAEFNKSQTS |
| | | FHLKKPSALVSDSALYFCAVRDQSGANNLFFGTGTRLTVIP |
| RAS TCR-2 | 25 | ATGGGCACCAGCCTCCTCTGCTGGATGGCCCTGTGTCTCCTGGGG |
| beta chain | | GCAGATCACGCAGATACTGGAGTCTCCCAGGACCCCAGACACAA |
| variable | | GATCACAAAGAGGGGACAGAATGTAACTTTCAGGTGTGATCCAA |
| domain | | TTTCTGAACACAACCGCCTTTATTGGTACCGACAGACCCTGGGGC |
| | | AGGGCCCAGAGTTTCTGACTTACTTCCAGAATGAAGCTCAACTAG |
| | | AAAAATCAAGGCTGCTCAGTGATCGGTTCTCTGCAGAGAGGCCT |
| | | AAGGGATCTTTCTCCACCTTGGAGATCCAGCGCACAGAGCAGGG |
| | | GGACTCGGCCATGTATCTCTGTGCCAGCTACCTGAGCGGTTCCAT |
| | | TTACAATGAGCAGTTCTTCGGGCCAGGGACACGGCTCACCGTGCT |
| | | A |
| | 26 | ATGGGCACTAGCCTCTTGTGTTGGATGGCACTTTGCCTTCTTGGC |
| | | GCGGATCACGCCGATACAGGCGTCTCCCAAGATCCCAGACATAA |
| | | AATCACAAAACGGGGCCAGAACGTTACCTTTCGCTGCGATCCGA |
| | | TATCAGAGCATAATCGACTGTATTGGTATAGGCAAACTCTCGGGC |
| | | AAGGGCCTGAGTTCCTCACTTATTTCCAAAATGAGGCGCAACTGG |
| | | AAAAGAGCCGGTTGTTGAGTGATAGGTTTTCCGCAGAGCGACCC |
| | | AAGGGGAGCTTCTCAACACTGGAGATACAAAGGACCGAACAAGG |
| | | TGATTCCGCAATGTATCTCTGTGCTAGTTATTTGAGCGGCTCCATA |
| | | TATAACGAACAGTTTTTCGGACCGGGCACTCGCCTGACCGTACTA |
| | 27 | MGTSLLCWMALCLLGADHADTGVSQDPRHKITKRGQNVTFRCDPIS |
| | | EHNRLYWYRQTLGQGPEFLTYFQNEAQLEKSRLLSDRFSAERPKGS |
| | | FSTLEIQRTEQGDSAMYLCASYLSGSIYNEQFFGPGTRLTVL |
| RAS TCR-2 | 28 | MASAPISMLAMLFTLSGLRAQSVAQPEDQVNVAEGNPLTVKCTYSV |
| alpha chain | | SGNPYLFWYVQYPNRGLQFLLKYITGDNLVKGSYGFEAEFNKSQTS |
| | | FHLKKPSALVSDSALYFCAVRDQSGANNLFFGTGTRLTVIPDIQNPD |
| | | PAVYQLRDSKSSDKSVCLFTDFDSQTNVSQSKDSDVYITDKTVLDM |
| | | RSMDFKSNSAVAWSNKSDFACANAFNNSIIPEDTFFPSPESSCDVKL |
| | | VEKSFETDTNLNFQNLSVIGFRILLLKVAGFNLLMTLRLWSS |
| RAS TCR-2 | 29 | MGTSLLCWMALCLLGADHADTGVSQDPRHKITKRGQNVTFRCDPIS |
| beta chain | | EHNRLYWYRQTLGQGPEFLTYFQNEAQLEKSRLLSDRFSAERPKGS |
| | | FSTLEIQRTEQGDSAMYLCASYLSGSIYNEQFFGPGTRLTVLEDLNK |
| | | VFPPEVAVFEPSEAEISHTQKATLVCLATGFFPDHVELSWWVNGKE |
| | | VHSGVSTDPQPLKEQPALNDSRYCLSSRLRVSATFWQNPRNHFRCQ |
| | | VQFYGLSENDEWTQDRAKPVTQIVSAEAWGRADCGFTSVSYQQGV |
| | | LSATILYEILLGKATLYAVLVSALVLMAMVKRKDF |
| RAS TCR- | 30 | LVVVGACGV |
| 1/2 peptide | | |
| Ras TCR-3 | 31 | NSMFDY |
| alpha chain | | |
| CDR1 | | |
| RAS TCR-3 | 32 | ISSIKDK |
| alpha chain | | |
| CDR2 | | |
| RAS TCR-3 | 33 | AASGGGGADGLT |
| alpha chain | | |
| CDR3 | | |
| RAS TCR-3 | 34 | MNHNY |
| beta chain | | |
| CDR1 | | |
| RAS TCR-3 | 35 | SVGAGI |
| beta chain | | |
| CDR2 | | |
| RAS TCR-3 | 36 | ASSYSTERGTIY |
| beta chain | | |
| CDR3 | | |
| RAS TCR-3 | 37 | ATGGCCATGCTCCTGGGGGCATCAGTGCTGATTCTGTGGCTTCAG |
| alpha | | CCAGACTGGGTAAACAGTCAACAGAAGAATGATGACCAGCAAGT |
| chain | | TAAGCAAAATTCACCATCCCTGAGCGTCCAGGAAGGAAGAATTT |
| variable | | CTATTCTGAACTGTGACTATACTAACAGCATGTTTGATTATTTCCT |
| domain | | ATGGTACAAAAAATACCCTGCTGAAGGTCCTACATTCCTGATATC |
| | | TATAAGTTCCATTAAGGATAAAAATGAAGATGGAAGATTCACTG |
| | | TCTTCTTAAACAAAAGTGCCAAGCACCTCTCTCTGCACATTGTGC |
| | | CCTCCCAGCCTGGAGACTCTGCAGTGTACTTCTGTGCAGCAAGCG |
| | | GGGGAGGAGGTGCTGACGGACTCACCTTTGGCAAAGGGACTCAT |
| | | CTAATCATCCAGCCC |
| | 38 | ATGGCCATGCTGCTGGGCGCCAGCGTGCTGATTTTATGGCTGCAG |
| | | CCCGACTGGGTGAACAGCCAGCAGAAGAACGACGACCAGCAAGT |
| | | GAAGCAGAACTCCCCTTCTTTAAGCGTGCAAGAAGGTCGTATCAG |
| | | CATTTTAAACTGCGACTACACCAACAGCATGTTCGACTACTTTTT |
| | | ATGGTACAAGAAGTACCCCGCCGAGGGCCCCACCTTTTTAATCAG |
| | | CATCAGCAGCATCAAGGACAAGAACGAGGACGGTCGTTTCACCG |
| | | TGTTTTTAAACAAGAGCGCCAAGCATTTATCTTTACACATCGTGC |
| | | CCTCCCAGCCCGGTGATAGCGCCGTGTACTTCTGCGCCGCCAGCG |
| | | GAGGAGGAGGCGCCGATGGACTGACCTTCGGCAAGGGCACCCAT |
| | | TTAATCATCCAGCCC |
| | 39 | MAMLLGASVLILWLQPDWVNSQQKNDDQQVKQNSPSLSVQEGRISI |
| | | LNCDYTNSMFDYFLWYKKYPAEGPTFLISISSIKDKNEDGRFTVFLN |
| | | KSAKHLSLHIVPSQPGDSAVYFCAASGGGGADGLTFGKGTHLIIQP |
| RAS TCR-3 | 40 | ATGAGGTCTCAGAATGACTTCCTTGAGAGTCCTGTTCCCCTTTCAT |
| beta chain | | CAATGCACAGATACAGAAGACCCCTCCGTCCTGGAGCACCTGCC |
| variable | | ATGAGCATCAGCCTCCTGTGCTGTGCAGCCTTTCCTCTCCTGTGG |
| domain | | GCAGGTCCAGTGAATGCTGGTGTCACTCAGACCCCAAAATTCCGC |
| | | ATCCTGAAGATAGGACAGAGCATGACACTGCAGTGTACCCAGGA |
| | | TATGAACCATAACTACATGTACTGGTATCGACAAGACCCAGGCAT |
| | | GGGGCTGAAGCTGATTTATTATTCAGTTGGTGCTGGTATCACTGA |
| | | TAAAGGAGAAGTCCCGAATGGCTACAACGTCTCCAGATCAACCA |
| | | CAGAGGATTTCCCGCTCAGGCTGGAGTTGGCTGCTCCCTCCCAGA |
| | | CATCTGTGTACTTCTGTGCCAGCAGTTACTCGACGGAACGCGGGA |
| | | CCATATATTTTGGAGAGGGAAGTTGGCTCACTGTTGTA |
| | 41 | ATGAGGAGCCAGAACGACTTTTTAGAGAGCCCCGTGCCTCTGAG |
| | | CAGCATGCATAGGTATAGGAGGCCTCTGAGACCCGGTGCCCCCG |
| | | CTATGAGCATCTCTTTACTGTGCTGTGCTGCCTTTCCTTTACTGTG |
| | | GGCTGGCCCCGTTAACGCTGGCGTGACCCAGACCCCCAAGTTTAG |
| | | GATTTTAAAGATCGGCCAGTCCATGACTTTACAGTGCACCCAAGA |
| | | TATGAACCACAACTACATGTACTGGTATCGTCAAGATCCCGGCAT |
| | | GGGTTTAAAGCTGATTTACTACAGCGTGGGAGCCGGCATCACCG |
| | | ACAAGGGCGAGGTGCCCAACGGCTACAATGTGTCTCGTAGCACC |
| | | ACCGAGGACTTCCCTCTGAGACTGGAGCTGGCCGCCCCTAGCCA |
| | | GACAAGCGTGTACTTCTGCGCCTCCTCCTACAGCACCGAGAGGG |
| | | GCACCATCTACTTCGGCGAGGGCAGCTGGCTGACCGTGGTG |
| | 42 | MRSQNDFLESPVPLSSMHRYRRPLRPGAPAMSISLLCCAAFPLLW |
| | | AGPVNAGVTQTPKFRILKIGQSMTLQCTQDMNHNYMYWYRQDPGM |
| | | GLKLIYYSVGAGITDKGEVPNGYNVSRSTTEDFPLRLELAAPSQT |
| | | SVYFCASSYSTERGTIYFGEGSWLTVV |
| RAS TCR-3 | 43 | MAMLLGASVLILWLQPDWVNSQQKNDDQQVKQNSPSLSVQEGRISI |
| alpha chain | | LNCDYTNSMFDYFLWYKKYPAEGPTFLISISSIKDKNEDGRFTVFLN |
| | | KSAKHLSLHIVPSQPGDSAVYFCAASGGGGADGLTFGKGTHLIIQPDI |
| | | QNPDPAVYQLRDSKSSDKSVCLFTDFDSQTNVSQSKDSDVYITDKT |
| | | VLDMRSMDFKSNSAVAWSNKSDFACANAFNNSIIPEDTFFPSPESSC |
| | | DVKLVEKSFETDTNLNFQNLSVIGFRILLLKVAGFNLLMTLRLWSS |
| RAS TCR-3 | 44 | MRSQNDFLESPVPLSSMHRYRRPLRPGAPAMSISLLCCAAFPLLWAG |
| beta chain | | PVNAGVTQTPKFRILKIGQSMTLQCTQDMNHNYMYWYRQDPGMG |
| | | LKLIYYSVGAGITDKGEVPNGYNVSRSTTEDFPLRLELAAPSQTSVY |
| | | FCASSYSTERGTIYFGEGSWLTVVEDLNKVFPPEVAVFEPSEAEISHT |
| | | QKATLVCLATGFFPDHVELSWWVNGKEVHSGVSTDPQPLKEQPAL |
| | | NDSRYCLSSRLRVSATFWQNPRNHFRCQVQFYGLSENDEWTQDRA |
| | | KPVTQIVSAEAWGRADCGFTSVSYQQGVLSATILYEILLGKATLYAV |
| | | LVSALVLMAMVKRKDF |
| RAS TCR-3 | 45 | VVGAVGVGK |
| peptide | 46 | VVVGAVGVGK |
| RAS TCR-4 | 47 | TSINN |
| alpha chain | | |
| CDR1 | | |
| RAS TCR-4 | 48 | IRSNERE |
| alpha chain | | |
| CDR2 | | |
| RAS TCR-4 | 49 | ATDRQSSGDKLT |
| alpha chain | | |
| CDR3 | | |
| RAS TCR-4 | 50 | SGHAT |
| beta chain | | |
| CDR1 | | |
| RAS TCR-4 | 51 | FQNNGV |
| beta chain | | |
| CDR2 | | |
| RAS TCR-4 | 52 | ASSLADIYEQY |
| beta chain | | |
| CDR3 | | |
| RAS TCR-4 | 53 | ATGGAAACTCTCCTGGGAGTGTCTTTGGTGATTCTATGGCTTCAA |
| alpha chain | | CTGGCTAGGGTGAACAGTCAACAGGGAGAAGAGGATCCTCAGGC |
| variable | | CTTGAGCATCCAGGAGGGTGAAAATGCCACCATGAACTGCAGTT |
| domain | | ACAAAACTAGTATAAACAATTTACAGTGGTATAGACAAAATTCA |
| | | GGTAGAGGCCTTGTCCACCTAATTTTAATACGTTCAAATGAAAGA |
| | | GAGAAACACAGTGGAAGATTAAGAGTCACGCTTGACACTTCCAA |
| | | GAAAAGCAGTTCCTTGTTGATCACGGCTTCCCGGGCAGCAGACA |
| | | CTGCTTCTTACTTCTGTGCTACGGACCGTCAAAGCAGCGGAGACA |
| | | AGCTGACTTTTGGGACCGGGACTCGTTTAGCAGTTAGGCCC |
| | 54 | ATGGAGACTTTACTGGGCGTGTCTTTAGTGATTTTATGGCTGCAG |
| | | CTGGCTCGTGTGAATAGCCAGCAAGGTGAAGAGGACCCCCAAGC |
| | | TTTAAGCATCCAAGAAGGCGAGAACGCCACCATGAACTGCTCCT |
| | | ACAAGACCAGCATCAACAATTTACAGTGGTATCGTCAGAACAGC |
| | | GGTCGTGGTTTAGTGCATTTAATTTTAATTCGTAGCAACGAGAGG |
| | | GAGAAGCACAGCGGTCGTCTGAGGGTGACTTTAGACACCAGCAA |
| | | GAAGAGCAGCTCTTTACTGATCACAGCCTCTAGGGCCGCTGACAC |
| | | CGCTAGCTACTTCTGCGCCACCGACAGACAGAGCAGCGGCGACA |
| | | AGCTGACCTTCGGCACCGGCACAAGACTGGCCGTGAGACCC |
| | 55 | METLLGVSLVILWLQLARVNSQQGEEDPQALSIQEGENATMNCS |
| | | YKTSINNLQWYRQNSGRGLVHLILIRSNEREKHSGRLRVTLDTS |
| | | KKSSSLLITASRAADTASYFCATDRQSSGDKLTFGTGTRLAVRP |
| RAS TCR-4 | 56 | ATGGGCACCAGGCTCCTCTGCTGGGCGGCCCTCTGTCTCCTGGGA |
| beta chain | | GCAGAACTCACAGAAGCTGGAGTTGCCCAGTCTCCCAGATATAA |
| variable | | GATTATAGAGAAAAGGCAGAGTGTGGCTTTTTGGTGCAATCCTAT |
| domain | | ATCTGGCCATGCTACCCTTTACTGGTACCAGCAGATCCTGGGACA |
| | | GGGCCCAAAGCTTCTGATTCAGTTTCAGAATAACGGTGTAGTGGA |
| | | TGATTCACAGTTGCCTAAGGATCGATTTTCTGCAGAGAGGCTCAA |
| | | AGGAGTAGACTCCACTCTCAAGATCCAGCCTGCAAAGCTTGAGG |
| | | ACTCGGCCGTGTATCTCTGTGCCAGCAGCTTAGCCGACATCTACG |
| | | AGCAGTACTTCGGGCCGGGCACCAGGCTCACGGTCACA |
| | 57 | ATGGGCACCAGACTGCTGTGCTGGGCCGCTCTGTGTCTGCTGGGC |
| | | GCTGAGCTGACAGAAGCTGGCGTGGCCCAGAGCCCTCGTTACAA |
| | | GATCATCGAGAAGAGGCAGAGCGTGGCCTTCTGGTGCAACCCCA |
| | | TCAGCGGCCACGCCACTTTATACTGGTACCAGCAGATTTTAGGCC |
| | | AAGGTCCCAAGCTGCTGATCCAGTTCCAGAACAACGGCGTGGTG |
| | | GACGACAGCCAGCTGCCCAAGGATCGTTTCAGCGCCGAGAGGCT |
| | | GAAGGGCGTGGACAGCACTTTAAAAATCCAGCCCGCTAAGCTGG |
| | | AGGACAGCGCCGTGTATTTATGCGCTAGCTCTTTAGCCGACATCT |
| | | ACGAGCAGTACTTCGGCCCCGGCACTCGTCTGACCGTGACC |
| | 58 | MGTRLLCWAALCLLGAELTEAGVAQSPRYKIIEKRQSVAFWCNPIS |
| | | GHATLYWYQQILGQGPKLLIQFQNNGVVDDSQLPKDRFSAERLKGV |
| | | DSTLKIQPAKLEDSAVYLCASSLADIYEQYFGPGTRLTVT |
| RAS TCR-4 | 59 | METLLGVSLVILWLQLARVNSQQGEEDPQALSIQEGENATMNCSYK |
| alpha chain | | TSINNLQWYRQNSGRGLVHLILIRSNEREKHSGRLRVTLDTSKKSSSL |
| | | LITASRAADTASYFCATDRQSSGDKLTFGTGTRLAVRPDIQNPDPAV |
| | | YQLRDSKSSDKSVCLFTDFDSQTNVSQSKDSDVYITDKTVLDMRSM |
| | | DFKSNSAVAWSNKSDFACANAFNNSIIPEDTFFPSPESSCDVKLVEKS |
| | | FETDTNLNFQNLSVIGFRILLLKVAGFNLLMTLRLWSS |
| RAS TCR-4 | 60 | MGTRLLCWAALCLLGAELTEAGVAQSPRYKIIEKRQSVAFWCNPIS |
| beta chain | | GHATLYWYQQILGQGPKLLIQFQNNGVVDDSQLPKDRFSAERLKGV |
| | | DSTLKIQPAKLEDSAVYLCASSLADIYEQYFGPGTRLTVTEDLNKVF |
| | | PPEVAVFEPSEAEISHTQKATLVCLATGFFPDHVELSWWVNGKEVH |
| | | SGVSTDPQPLKEQPALNDSRYCLSSRLRVSATFWQNPRNHFRCQVQ |
| | | FYGLSENDEWTQDRAKPVTQIVSAEAWGRADCGFTSVSYQQGVLS |
| | | ATILYEILLGKATLYAVLVSALVLMAMVKRKDF |
| RAS TCR-4 | 61 | VVGAVGVGK |
| peptide | 62 | VVVGAVGVGK |
| RAS TCR-5 | 63 | TSGFNG |
| alpha chain | | |
| CDR1 | | |
| RAS TCR-5 | 64 | NVLDGL |
| alpha chain | | |
| CDR2 | | |
| RAS TCR-5 | 65 | CAPGDNFNKFYF |
| alpha chain | | |
| CDR3 | | |
| RAS TCR-5 | 66 | SGHRS |
| beta chain | | |
| CDR1 | | |
| RAS TCR-5 | 67 | YFSETQ |
| beta chain | | |
| CDR2 | | |
| RAS TCR-5 | 68 | CASSARNDEAFF |
| beta chain | | |
| CDR3 | | |
| RAS TCR-5 | 69 | ATGTGGGGAGTTTTCCTTCTTTATGTTTCCATGAAGATGGGAGGC |
| alpha chain | | ACTACAGGACAAAACATTGACCAGCCCACTGAGATGACAGCTAC |
| variable | | GGAAGGTGCCATTGTCCAGATCAACTGCACGTACCAGACATCTG |
| domain | | GGTTCAACGGGCTGTTCTGGTACCAGCAACATGCTGGCGAAGCA |
| | | CCCACATTTCTGTCTTACAATGTTCTGGATGGTTTGGAGGAGAAA |
| | | GGTCGTTTTTCTTCATTCCTTAGTCGGTCTAAAGGGTACAGTTACC |
| | | TCCTTTTGAAGGAGCTCCAGATGAAAGACTCTGCCTCTTACCTCT |
| | | GTGCTCCCGGGGACAACTTCAACAAATTTTACTTTGGATCTGGGA |
| | | CCAAACTCAATGTAAAACCA |
| | 70 | ATGTGGGGCGTGTTTCTGCTGTACGTGTCCATGAAGATGGGCGGC |
| | | ACCACAGGCCAGAACATCGACCAGCCAACCGAGATGACCGCCAC |
| | | AGAGGGCGCCATCGTGCAGATCAACTGCACCTACCAGACATCTG |
| | | GCTTCAATGGCCTGTTTTGGTATCAGCAGCACGCAGGAGAGGCAC |
| | | CCACATTCCTGAGCTATAATGTGCTGGATGGCCTGGAGGAGAAG |
| | | GGCAGGTTCTCCTCTTTTCTGTCTCGCAGCAAGGGCTACTCCTATC |
| | | TGCTGCTGAAGGAGCTGCAGATGAAGGACTCCGCCTCTTACCTGT |
| | | GCGCCCCTGGCGATAACTTTAATAAGTTCTATTTCGGCTCTGGCA |
| | | CCAAGCTGAATGTGAAGCCA |
| | 71 | MWGVFLLYVSMKMGGTTGQNIDQPTEMTATEGAIVQINCTYQTSG |
| | | FNGLFWYQQHAGEAPTFLSYNVLDGLEEKGRFSSFLSRSKGYSYL |
| | | LLKELQMKDSASYLCAPGDNFNKFYFGSGTKLNVKP |
| RAS TCR-5 | 72 | ATGGGCTCCAGGCTGCTCTGTTGGGTGCTGCTTTGTCTCCTGGGA |
| beta chain | | GCAGGCCCAGTAAAGGCTGGAGTCACTCAAACTCCAAGATATCT |
| variable | | GATCAAAACGAGAGGACAGCAAGTGACACTGAGCTGCTCCCCTA |
| domain | | TCTCTGGGCATAGGAGTGTATCCTGGTACCAACAGACCCCAGGA |
| | | CAGGGCCTTCAGTTCCTCTTTGAATACTTCAGTGAGACACAGAGA |
| | | AACAAAGGAAACTTCCCTGGTCGATTCTCAGGGCGCCAGTTCTCT |
| | | AACTCTCGCTCTGAGATGAATGTGAGCACCTTGGAGCTGGGGGA |
| | | CTCGGCCCTTTATCTTTGCGCCAGCAGCGCGAGAAATGATGAAGC |
| | | TTTCTTTGGACAAGGCACCAGACTCACAGTTGTA |
| | 73 | ATGGGCAGCCGGCTGCTGTGCTGGGTGCTGCTGTGCCTGCTGGGA |
| | | GCAGGACCAGTGAAGGCAGGCGTGACCCAGACACCTCGGTACCT |
| | | GATCAAGACCAGAGGCCAGCAGGTGACACTGAGCTGCTCCCCAA |
| | | TCTCCGGCCACAGATCTGTGAGCTGGTACCAGCAGACCCCAGGA |
| | | CAGGGACTGCAGTTCCTGTTTGAGTATTTCTCCGAGACACAGAGG |
| | | AACAAGGGCAATTTCCCTGGCCGGTTTTCTGGCAGACAGTTTTCC |
| | | AACTCTCGCAGCGAGATGAATGTGAGCACCCTGGAGCTGGGCGA |
| | | CTCCGCCCTGTACCTGTGCGCCAGCTCCGCCAGGAACGATGAGGC |
| | | CTTCTTTGGCCAGGGCACCCGGCTGACAGTGGTG |
| | 74 | MGSRLLCWVLLCLLGAGPVKAGVTQTPRYLIKTRGQQVTLSCSPISG |
| | | HRSVSWYQQTPGQGLQFLFEYFSETQRNKGNFPGRFSGRQFSNSRSE |
| | | MNVSTLELGDSALYLCASSARNDEAFFGQGTRLTVV |
| RAS TCR-5 | 75 | MWGVFLLYVSMKMGGTTGQNIDQPTEMTATEGAIVQINCTYQTSG |
| alpha chain | | FNGLFWYQQHAGEAPTFLSYNVLDGLEEKGRFSSFLSRSKGYSYLLL |
| | | KELQMKDSASYLCAPGDNFNKFYFGSGTKLNVKPDIQNPDPAVYQL |
| | | RDSKSSDKSVCLFTDFDSQTNVSQSKDSDVYITDKTVLDMRSMDFK |
| | | SNSAVAWSNKSDFACANAFNNSIIPEDTFFPSPESSCDVKLVEKSFET |
| | | DTNLNFQNLSVIGFRILLLKVAGFNLLMTLRLWSS |
| RAS TCR-5 | 76 | MGSRLLCWVLLCLLGAGPVKAGVTQTPRYLIKTRGQQVTLSCSPISG |
| beta chain | | HRSVSWYQQTPGQGLQFLFEYFSETQRNKGNFPGRFSGRQFSNSRSE |
| | | MNVSTLELGDSALYLCASSARNDEAFFGQGTRLTVVEDLNKVFPPE |
| | | VAVFEPSEAEISHTQKATLVCLATGFFPDHVELSWWVNGKEVHSGV |
| | | STDPQPLKEQPALNDSRYCLSSRLRVSATFWQNPRNHFRCQVQFYG |
| | | LSENDEWTQDRAKPVTQIVSAEAWGRADCGFTSVSYQQGVLSATIL |
| | | YEILLGKATLYAVLVSALVLMAMVKRKDF |
| RAS TCR-5 | 77 | VVGAVGVGK |
| peptide | 78 | VVVGAVGVGK |
| RAS TCR-6 | 79 | DRGSQS |
| alpha chain | | |
| CDR1 | | |
| RAS TCR-6 | 80 | IYSNGD |
| alpha chain | | |
| CDR2 | | |
| RAS TCR-6 | 81 | CAVKSRAGSYQLTF |
| alpha chain | | |
| CDR3 | | |
| RAS TCR-6 | 82 | SGHNS |
| beta chain | | |
| CDR1 | | |
| RAS TCR-6 | 83 | FNNNVP |
| beta chain | | |
| CDR2 | | |
| RAS TCR-6 | 84 | CASSLGDSEQYF |
| beta chain | | |
| CDR3 | | |
| RAS TCR-6 | 85 | ATGAAATCCTTGAGAGTTTTACTAGTGATCCTGTGGCTTCAGTTG |
| alpha chain | | AGCTGGGTTTGGAGCCAACAGAAGGAGGTGGAGCAGAATTCTGG |
| variable | | ACCCCTCAGTGTTCCAGAGGGAGCCATTGCCTCTCTCAACTGCAC |
| domain | | TTACAGTGACCGAGGTTCCCAGTCCTTCTTCTGGTACAGACAATA |
| | | TTCTGGGAAAAGCCCTGAGTTGATAATGTTCATATACTCCAATGG |
| | | TGACAAAGAAGATGGAAGGTTTACAGCACAGCTCAATAAAGCCA |
| | | GCCAGTATGTTTCTCTGCTCATCAGAGACTCCCAGCCCAGTGATT |
| | | CAGCCACCTACCTCTGTGCCGTGAAGTCAAGGGCTGGGAGTTACC |
| | | AACTCACTTTCGGGAAGGGGACCAAACTCTCGGTCATACCA |
| | 86 | ATGAAGAGCCTGCGGGTGCTGCTGGTCATCCTGTGGCTGCAGCTG |
| | | TCCTGGGTGTGGTCTCAGCAGAAGGAGGTGGAGCAGAATAGCGG |
| | | ACCACTGTCCGTGCCAGAGGGAGCCATCGCCTCCCTGAACTGCAC |
| | | ATACTCTGACAGGGGCTCCCAGTCTTTCTTTTGGTACCGCCAGTA |
| | | TAGCGGCAAGTCCCCCGAGCTGATCATGTTCATCTACTCTAATGG |
| | | CGACAAGGAGGATGGCAGGTTTACCGCCCAGCTGAACAAGGCCT |
| | | CTCAGTATGTGAGCCTGCTGATCCGCGACAGCCAGCCTAGCGATT |
| | | CCGCCACATACCTGTGCGCAGTGAAGTCCCGGGCAGGCTCTTATC |
| | | AGCTGACCTTTGGCAAGGGCACAAAGCTGAGCGTGATCCCA |
| | 87 | MKSLRVLLVILWLQLSWVWSQQKEVEQNSGPLSVPEGAIASLNCTY |
| | | SDRGSQSFFWYRQYSGKSPELIMFIYSNGDKEDGRFTAQLNKASQY |
| | | VSLLIRDSQPSDSATYLCAVKSRAGSYQLTFGKGTKLSVIP |
| RAS TCR-6 | 88 | ATGGACTCCTGGACCTTCTGCTGTGTGTCCCTTTGCATCCTGGTAG |
| beta chain | | CGAAGCATACAGATGCTGGAGTTATCCAGTCACCCCGCCATGAG |
| variable | | GTGACAGAGATGGGACAAGAAGTGACTCTGAGATGTAAACCAAT |
| domain | | TTCAGGCCACAACTCCCTTTTCTGGTACAGACAGACCATGATGCG |
| | | GGGACTGGAGTTGCTCATTTACTTTAACAACAACGTTCCGATAGA |
| | | TGATTCAGGGATGCCCGAGGATCGATTCTCAGCTAAGATGCCTAA |
| | | TGCATCATTCTCCACTCTGAAGATCCAGCCCTCAGAACCCAGGGA |
| | | CTCAGCTGTGTACTTCTGTGCCAGCAGTCTCGGGGACAGCGAGCA |
| | | GTACTTCGGGCCGGGCACCAGGCTCACGGTCACA |
| | 89 | ATGGACAGCTGGACCTTCTGCTGCGTGAGCCTGTGCATCCTGGTG |
| | | GCCAAGCACACAGATGCAGGCGTGATCCAGTCCCCAAGGCACGA |
| | | GGTGACCGAGATGGGACAGGAGGTGACACTGAGGTGTAAGCCTA |
| | | TCTCTGGCCACAATAGCCTGTTCTGGTACAGGCAGACCATGATGC |
| | | GCGGCCTGGAGCTGCTGATCTACTTCAACAATAACGTGCCTATCG |
| | | ACGATTCCGGCATGCCAGAGGACAGATTCTCTGCCAAGATGCCC |
| | | AACGCCTCCTTTTCTACACTGAAGATCCAGCCAAGCGAGCCTAGG |
| | | GACTCCGCCGTGTACTTCTGCGCCAGCTCCCTGGGCGATAGCGAG |
| | | CAGTATTTTGGCCCTGGCACCCGGCTGACCGTGACA |
| | 90 | MDSWTFCCVSLCILVAKHTDAGVIQSPRHEVTEMGQEVTLRCKPISG |
| | | HNSLFWYRQTMMRGLELLIYFNNNVPIDDSGMPEDRFSAKMPNASF |
| | | STLKIQPSEPRDSAVYFCASSLGDSEQYFGPGTRLTVT |
| RAS TCR-6 | 91 | MKSLRVLLVILWLQLSWVWSQQKEVEQNSGPLSVPEGAIASLNCTY |
| alpha chain | | SDRGSQSFFWYRQYSGKSPELIMFIYSNGDKEDGRFTAQLNKASQY |
| | | VSLLIRDSQPSDSATYLCAVKSRAGSYQLTFGKGTKLSVIPDIQNPDP |
| | | AVYQLRDSKSSDKSVCLFTDFDSQTNVSQSKDSDVYITDKTVLDMR |
| | | SMDFKSNSAVAWSNKSDFACANAFNNSIIPEDTFFPSPESSCDVKLV |
| | | EKSFETDTNLNFQNLSVIGFRILLLKVAGFNLLMTLRLWSS |
| RAS TCR-6 | 92 | MDSWTFCCVSLCILVAKHTDAGVIQSPRHEVTEMGQEVTLRCKPISG |
| beta chain | | HNSLFWYRQTMMRGLELLIYFNNNVPIDDSGMPEDRFSAKMPNASF |
| | | STLKIQPSEPRDSAVYFCASSLGDSEQYFGPGTRLTVTEDLNKVFPPE |
| | | VAVFEPSEAEISHTQKATLVCLATGFFPDHVELSWWVNGKEVHSGV |
| | | STDPQPLKEQPALNDSRYCLSSRLRVSATFWQNPRNHFRCQVQFYG |
| | | LSENDEWTQDRAKPVTQIVSAEAWGRADCGFTSVSYQQGVLSATIL |
| | | YEILLGKATLYAVLVSALVLMAMVKRKDF |
| RAS TCR-6 | 93 | VVGAVGVGK |
| peptide | 94 | VVVGAVGVGK |
| RAS TCR-7 | 95 | SSYSPS |
| alpha chain | | |
| CDR1 | | |
| RAS TCR-7 | 96 | YTSAATLV |
| alpha chain | | |
| CDR2 | | |
| RAS TCR-7 | 97 | CVVSGGGSSNTGKLIF |
| alpha chain | | |
| CDR3 | | |
| RAS TCR-7 | 98 | SGHAT |
| beta chain | | |
| CDR1 | | |
| RAS TCR-7 | 99 | FQNNGV |
| beta chain | | |
| CDR2 | | |
| RAS TCR-7 | 100 | CASSQRSNTGELFF |
| beta chain | | |
| CDR3 | | |
| RAS TCR-7 | 101 | ATGCTCCTGCTGCTCGTCCCAGTGCTCGAGGTGATTTTTACTCTGG |
| alpha chain | | GAGGAACCAGAGCCCAGTCGGTGACCCAGCTTGACAGCCACGTC |
| variable | | TCTGTCTCTGAAGGAACCCCGGTGCTGCTGAGGTGCAACTACTCA |
| domain | | TCTTCTTATTCACCATCTCTCTTCTGGTATGTGCAACACCCCAACA |
| | | AAGGACTCCAGCTTCTCCTGAAGTACACATCAGCGGCCACCCTGG |
| | | TTAAAGGCATCAACGGTTTTGAGGCTGAATTTAAGAAGAGTGAA |
| | | ACCTCCTTCCACCTGACGAAACCCTCAGCCCATATGAGCGACGCG |
| | | GCTGAGTACTTCTGTGTTGTGAGTGGGGGAGGCTCTAGCAACACA |
| | | GGCAAACTAATCTTTGGGCAAGGGACAACTTTACAAGTAAAACC |
| | | A |
| | 102 | ATGCTGCTGCTGCTGGTGCCCGTGCTGGAAGTGATCTTCACCCTG |
| | | GGAGGAACAAGGGCACAGAGCGTGACCCAGCTGGACTCCCACGT |
| | | GTCCGTGTCTGAGGGCACACCCGTGCTGCTGAGATGCAACTACTC |
| | | CTCTAGCTATAGCCCCTCCCTGTTCTGGTACGTGCAGCACCCTAA |
| | | TAAGGGCCTGCAGCTGCTGCTGAAGTATACCTCCGCCGCCACACT |
| | | GGTGAAGGGCATCAACGGCTTCGAGGCCGAGTTTAAGAAGAGCG |
| | | AGACCTCCTTCCACCTGACAAAGCCTTCTGCCCACATGAGCGATG |
| | | CCGCCGAGTACTTTTGCGTGGTGAGCGGCGGCGGCTCCTCTAATA |
| | | CCGGCAAGCTGATCTTCGGCCAGGGCACCACACTGCAGGTGAAG |
| | | CCA |
| | 103 | MLLLLVPVLEVIFTLGGTRAQSVTQLDSHVSVSEGTPVLLRCNYSSS |
| | | YSPSLFWYVQHPNKGLQLLLKYTSAATLVKGINGFEAEFKKSETSFH |
| | | LTKPSAHMSDAAEYFCVVSGGGSSNTGKLIFGQGTTLQVKP |
| RAS TCR-7 | 104 | ATGGGCACCAGGCTCCTCTGCTGGGCGGCCCTCTGTCTCCTGGGA |
| beta chain | | GCAGAACTCACAGAAGCTGGAGTTGCCCAGTCTCCCAGATATAA |
| variable | | GATTATAGAGAAAAGGCAGAGTGTGGCTTTTTGGTGCAATCCTAT |
| domain | | ATCTGGCCATGCTACCCTTTACTGGTACCAGCAGATCCTGGGACA |
| | | GGGCCCAAAGCTTCTGATTCAGTTTCAGAATAACGGTGTAGTGGA |
| | | TGATTCACAGTTGCCTAAGGATCGATTTTCTGCAGAGAGGCTCAA |
| | | AGGAGTAGACTCCACTCTCAAGATCCAACCTGCAAAGCTTGAGG |
| | | ACTCGGCCGTGTATCTCTGTGCCAGCAGCCAGAGGTCGAACACC |
| | | GGGGAGCTGTTTTTTGGAGAAGGCTCTAGGCTGACCGTACTG |
| | 105 | ATGGGCACCCGGCTGCTGTGCTGGGCCGCCCTGTGCCTGCTGGGA |
| | | GCAGAGCTGACAGAGGCAGGAGTGGCCCAGTCCCCACGGTACAA |
| | | GATCATCGAGAAGAGACAGTCCGTGGCCTTTTGGTGCAACCCCAT |
| | | CTCTGGCCACGCCACCCTGTACTGGTATCAGCAGATCCTGGGCCA |
| | | GGGCCCTAAGCTGCTGATCCAGTTCCAGAACAATGGCGTGGTGG |
| | | ACGATTCTCAGCTGCCAAAGGACAGGTTTAGCGCCGAGCGCCTG |
| | | AAGGGCGTGGATAGCACCCTGAAGATCCAGCCTGCCAAGCTGGA |
| | | GGACAGCGCCGTGTATCTGTGCGCCAGCTCCCAGCGGTCCAATAC |
| | | AGGCGAGCTGTTCTTTGGCGAGGGCTCTAGGCTGACCGTGCTG |
| | 106 | MGTRLLCWAALCLLGAELTEAGVAQSPRYKIIEKRQSVAFWCNPIS |
| | | GHATLYWYQQILGQGPKLLIQFQNNGVVDDSQLPKDRFSAERLKGV |
| | | DSTLKIQPAKLEDSAVYLCASSQRSNTGELFFGEGSRLTVL |
| RAS TCR-7 | 107 | MLLLLVPVLEVIFTLGGTRAQSVTQLDSHVSVSEGTPVLLRCNYSSS |
| alpha chain | | YSPSLFWYVQHPNKGLQLLLKYTSAATLVKGINGFEAEFKKSETSFH |
| | | LTKPSAHMSDAAEYFCVVSGGGSSNTGKLIFGQGTTLQVKPDIQNPD |
| | | PAVYQLRDSKSSDKSVCLFTDFDSQTNVSQSKDSDVYITDKTVLDM |
| | | RSMDFKSNSAVAWSNKSDFACANAFNNSIIPEDTFFPSPESSCDVKL |
| | | VEKSFETDTNLNFQNLSVIGFRILLLKVAGFNLLMTLRLWSS |
| RAS TCR-7 | 108 | MGTRLLCWAALCLLGAELTEAGVAQSPRYKIIEKRQSVAFWCNPIS |
| beta chain | | GHATLYWYQQILGQGPKLLIQFQNNGVVDDSQLPKDRFSAERLKGV |
| | | DSTLKIQPAKLEDSAVYLCASSQRSNTGELFFGEGSRLTVLEDLNKV |
| | | FPPEVAVFEPSEAEISHTQKATLVCLATGFFPDHVELSWWVNGKEV |
| | | HSGVSTDPQPLKEQPALNDSRYCLSSRLRVSATFWQNPRNHFRCQV |
| | | QFYGLSENDEWTQDRAKPVTQIVSAEAWGRADCGFTSVSYQQGVL |
| | | SATILYEILLGKATLYAVLVSALVLMAMVKRKDF |
| RAS TCR-7 | 109 | VVGACGVGK |
| peptide | 110 | VVVGACGVGK |
| RAS TCR-8 | 111 | TSINN |
| alpha chain | | |
| CDR1 | | |
| RAS TCR-8 | 112 | IRSNERE |
| alpha chain | | |
| CDR2 | | |
| RAS TCR-8 | 113 | CATDAGGGADGLTF |
| alpha chain | | |
| CDR3 | | |
| RAS TCR-8 | 114 | SGDLS |
| beta chain | | |
| CDR1 | | |
| RAS TCR-8 | 115 | YYNGEE |
| beta chain | | |
| CDR2 | | |
| RAS TCR-8 | 116 | CASGGRDSTDTQYF |
| beta chain | | |
| CDR3 | | |
| RAS TCR-8 | 117 | ATGGAAACTCTCCTGGGAGTGTCTTTGGTGATTCTATGGCTTCAA |
| alpha chain | | CTGGCTAGGGTGAACAGTCAACAGGGAGAAGAGGATCCTCAGGC |
| variable | | CTTGAGCATCCAGGAGGGTGAAAATGCCACCATGAACTGCAGTT |
| domain | | ACAAAACTAGTATAAACAATTTACAGTGGTATAGACAAAATTCA |
| | | GGTAGAGGCCTTGTCCACCTAATTTTAATACGTTCAAATGAAAGA |
| | | GAGAAACACAGTGGAAGATTAAGAGTCACGCTTGACACTTCCAA |
| | | GAAAAGCAGTTCCTTGTTGATCACGGCTTCCCGGGCAGCAGACA |
| | | CTGCTTCTTACTTCTGTGCTACGGACGCCGGAGGAGGTGCTGACG |
| | | GACTCACCTTTGGCAAAGGGACTCATCTAATCATCCAGCCC |
| | 118 | ATGGAGACACTGCTGGGCGTGTCCCTGGTCATCCTGTGGCTGCAG |
| | | CTGGCCAGGGTGAACAGCCAGCAGGGAGAGGAGGACCCCCAGG |
| | | CCCTGTCTATCCAGGAGGGCGAGAACGCCACCATGAATTGCTCTT |
| | | ACAAGACAAGCATCAACAATCTGCAGTGGTATAGACAGAACTCC |
| | | GGCAGGGGCCTGGTGCACCTGATCCTGATCCGCTCCAATGAGCG |
| | | GGAGAAGCACTCTGGCCGGCTGAGAGTGACCCTGGATACATCTA |
| | | AGAAGTCCTCTAGCCTGCTGATCACCGCCAGCCGGGCAGCAGAC |
| | | ACAGCCTCCTACTTTTGTGCCACCGATGCCGGGGGCGGAGCAGA |
| | | CGGACTGACATTCGGGAAGGGGACTCACCTGATTATCCAGCCA |
| | 119 | METLLGVSLVILWLQLARVNSQQGEEDPQALSIQEGENATMNCS |
| | | YKTSINNLQWYRQNSGRGLVHLILIRSNEREKHSGRLRVTLDTS |
| | | KKSSSLLITASRAADTASYFCATDAGGGADGLTFGKGTHLIIQP |
| RAS TCR-8 | 120 | ATGGGCTTCAGGCTCCTCTGCTGTGTGGCCTTTTGTCTCCTGGGAG |
| beta chain | | CAGGCCCAGTGGATTCTGGAGTCACACAAACCCCAAAGCACCTG |
| variable | | ATCACAGCAACTGGACAGCGAGTGACGCTGAGATGCTCCCCTAG |
| domain | | GTCTGGAGACCTCTCTGTGTACTGGTACCAACAGAGCCTGGACCA |
| | | GGGCCTCCAGTTCCTCATTCAGTATTATAATGGAGAAGAGAGAGC |
| | | AAAAGGAAACATTCTTGAACGATTCTCCGCACAACAGTTCCCTGA |
| | | CTTGCACTCTGAACTAAACCTGAGCTCTCTGGAGCTGGGGGACTC |
| | | AGCTTTGTATTTCTGTGCCAGCGGGGGACGGGATTCCACAGATAC |
| | | GCAGTATTTTGGCCCAGGCACCCGGCTGACAGTGCTC |
| | 121 | ATGGGCTTTCGGCTGCTGTGCTGCGTGGCTTTTTGCCTGCTGGGG |
| | | GCTGGGCCTGTGGATAGCGGGGTCACTCAGACACCTAAACATCT |
| | | GATCACCGCAACAGGACAGAGGGTGACCCTGAGGTGCTCTCCTC |
| | | GGAGCGGCGACCTGAGCGTGTACTGGTATCAGCAGAGCCTGGAT |
| | | CAGGGCCTGCAGTTCCTGATCCAGTACTATAACGGCGAGGAGCG |
| | | CGCCAAGGGCAATATCCTGGAGCGGTTCTCTGCCCAGCAGTTTCC |
| | | AGACCTGCACAGCGAGCTGAACCTGAGCTCCCTGGAGCTGGGCG |
| | | ATAGCGCCCTGTACTTCTGCGCCTCCGGCGGCAGAGACTCTACCG |
| | | ATACACAGTATTTTGGCCCCGGCACCAGACTGACAGTGCTG |
| | 122 | MGFRLLCCVAFCLLGAGPVDSGVTQTPKHLITATGQRVTLRCSPRSG |
| | | DLSVYWYQQSLDQGLQFLIQYYNGEERAKGNILERFSAQQFPDLHS |
| | | ELNLSSLELGDSALYFCASGGRDSTDTQYFGPGTRLTVL |
| RAS TCR-8 | 123 | METLLGVSLVILWLQLARVNSQQGEEDPQALSIQEGENATMNCSYK |
| alpha chain | | TSINNLQWYRQNSGRGLVHLILIRSNEREKHSGRLRVTLDTSKKSSSL |
| | | LITASRAADTASYFCATDAGGGADGLTFGKGTHLIIQPDIQNPDPAV |
| | | YQLRDSKSSDKSVCLFTDFDSQTNVSQSKDSDVYITDKTVLDMRSM |
| | | DFKSNSAVAWSNKSDFACANAFNNSIIPEDTFFPSPESSCDVKLVEKS |
| | | FETDTNLNFQNLSVIGFRILLLKVAGENLLMTLRLWSS |
| RAS TCR-8 | 124 | MGFRLLCCVAFCLLGAGPVDSGVTQTPKHLITATGQRVTLRCSPRSG |
| beta chain | | DLSVYWYQQSLDQGLQFLIQYYNGEERAKGNILERFSAQQFPDLHS |
| | | ELNLSSLELGDSALYFCASGGRDSTDTQYFGPGTRLTVLEDLNKVFP |
| | | PEVAVFEPSEAEISHTQKATLVCLATGFFPDHVELSWWVNGKEVHS |
| | | GVSTDPQPLKEQPALNDSRYCLSSRLRVSATFWQNPRNHFRCQVQF |
| | | YGLSENDEWTQDRAKPVTQIVSAEAWGRADCGFTSVSYQQGVLSA |
| | | TILYEILLGKATLYAVLVSALVLMAMVKRKDF |
| RAS TCR-8 | 125 | VVGAVGVGK |
| peptide | 126 | VVVGAVGVGK |
| Ras TCR-1/2 | 219 | KLVVVGADGV |
| peptide | | |
| Ras TCR-1/2 | 220 | LVVVGADGV |
| peptide | | |
| Ras TCR-1/2 | 221 | KLVVVGAVGV |
| peptide | | |
| Ras TCR-1/2 | 222 | LVVVGAVGV |
| peptide | | |
| RAS TCR-10 | 239 | SVFSS |
| alpha chain | | |
| CDR1 | | |
| RAS TCR-10 | 240 | VVTGGEV |
| alpha chain | | |
| CDR2 | | |
| RAS TCR-10 | 241 | AGGPNTGNQFY |
| alpha chain | | |
| CDR3 | | |
| RAS TCR-10 | 242 | SGHNT |
| beta chain | | |
| CDR1 | | |
| RAS TCR-10 | 243 | YYREEE |
| beta chain | | |
| CDR2 | | |
| RAS TCR-10 | 244 | ASSTSFWEVNTEAF |
| beta chain | | |
| CDR3 | | |
| RAS TCR-10 | 245 | ATGGTCCTGAAATTCTCCGTGTCCATTCTTTGGATTCAGTTGGCA |
| alpha chain | | TGGGTGAGCACCCAGCTGCTGGAGCAGAGCCCTCAGTTTCTAAGC |
| variable | | ATCCAAGAGGGAGAAAATCTCACTGTGTACTGCAACTCCTCAAGT |
| domains | | GTTTTTTCCAGCTTACAATGGTACAGACAGGAGCCTGGGGAAGGT |
| | | CCTGTCCTCCTGGTGACAGTAGTTACGGGTGGAGAAGTGAAGAA |
| | | GCTGAAGAGACTAACCTTTCAGTTTGGTGATGCAAGAAAGGACA |
| | | GTTCTCTCCACATCACTGCGGCCCAGCCTGGTGATACAGGCCTCT |
| | | ACCTCTGTGCAGGAGGGCCGAACACCGGTAACCAGTTCTATTTTG |
| | | GGACAGGGACAAGTTTGACGGTCATTCCAAAT |
| | 246 | ATGGTGCTGAAGTTTTCCGTGTCTATCCTGTGGATTCAGCTGGCCT |
| | | GGGTGTCTACCCAGCTGCTGGAGCAGAGCCCCCAGTTCCTGTCCA |
| | | TCCAGGAGGGCGAGAACCTGACAGTGTACTGCAATTCTAGCTCC |
| | | GTGTTTTCTAGCCTGCAGTGGTATAGGCAGGAGCCAGGAGAGGG |
| | | ACCCGTGCTGCTGGTGACCGTGGTGACAGGCGGCGAGGTGAAGA |
| | | AGCTGAAGAGACTGACCTTCCAGTTTGGCGACGCCAGGAAGGAT |
| | | TCCTCTCTGCACATCACCGCAGCACAGCCTGGCGATACAGGACTG |
| | | TACCTGTGCGCAGGAGGACCAAACACCGGCAATCAGTTCTATTTT |
| | | GGCACCGGCACATCCCTGACAGTGATCCCTAAT |
| | 247 | MVLKFSVSILWIQLAWVSTQLLEQSPQFLSIQEGENLTVYCNSSS |
| | | VFSSLQWYRQEPGEGPVLLVTVVTGGEVKKLKRLTFQFGDARKDS |
| | | SLHITAAQPGDTGLYLCAGGPNTGNQFYFGTGTSLTVIPN |
| RAS TCR-10 | 248 | ATGGGCCCTGGGCTCCTCTGCTGGGTGCTGCTTTGTCTCCTGGGA |
| beta chain | | GCAGGCTCAGTGGAGACTGGAGTCACCCAAAGTCCCACACACCT |
| variable | | GATCAAAACGAGAGGACAGCAAGTGACTCTGAGATGCTCTTCTC |
| domain | | AGTCTGGGCACAACACTGTGTCCTGGTACCAACAGGCCCTGGGTC |
| | | AGGGGCCCCAGTTTATCTTTCAGTATTATAGGGAGGAAGAGAAT |
| | | GGCAGAGGAAACTTCCCTCCTAGATTCTCAGGTCTCCAGTTCCCT |
| | | AATTATAGCTCTGAGCTGAATGTGAACGCCTTGGAGCTGGACGAC |
| | | TCGGCCCTGTATCTCTGTGCCAGCAGCACATCTTTTTGGGAGGTG |
| | | AACACTGAAGCTTTCTTTGGACAAGGCACCAGACTCACAGTTGTA |
| | 249 | ATGGGACCAGGACTGCTGTGCTGGGTGCTGCTGTGCCTGCTGGGA |
| | | GCAGGCTCCGTGGAGACCGGCGTGACACAGTCTCCCACCCACCT |
| | | GATCAAGACAAGAGGCCAGCAGGTGACCCTGAGGTGCAGCTCCC |
| | | AGTCTGGCCACAACACAGTGAGCTGGTACCAGCAGGCCCTGGGA |
| | | CAGGGACCTCAGTTCATCTTTCAGTACTATAGGGAGGAGGAGAA |
| | | CGGCCGCGGCAATTTCCCCCCTCGGTTTAGCGGCCTGCAGTTCCC |
| | | AAACTACTCTAGCGAGCTGAACGTGAATGCCCTGGAGCTGGACG |
| | | ATAGCGCCCTGTATCTGTGCGCCTCCTCTACCTCCTTTTGGGAAGT |
| | | GAATACAGAGGCCTTCTTTGGCCAGGGCACCCGCCTGACAGTGG |
| | | TG |
| | 250 | MGPGLLCWVLLCLLGAGSVETGVTQSPTHLIKTRGQQVTLRCSSQS |
| | | GHNTVSWYQQALGQGPQFIFQYYREEENGRGNFPPRFSGLQFPNYS |
| | | SELNVNALELDDSALYLCASSTSFWEVNTEAFFGQGTRLTVV |
| RAS TCR-10 | 251 | MVLKFSVSILWIQLAWVSTQLLEQSPQFLSIQEGENLTVYCNSSSVF |
| alpha chain | | SSLQWYRQEPGEGPVLLVTVVTGGEVKKLKRLTFQFGDARKDSSLHI |
| | | TAAQPGDTGLYLCAGGPNTGNQFYFGTGTSLTVIPNDIQNPEPAVYQ |
| | | LKDPRSQDSTLCLFTDFDSQINVPKTMESGTFITDKTVLDMKAMDSK |
| | | SNGAIAWSNQTSFTCQDIFKETNATYPSSDVPCDATLTEKSFETDMN |
| | | LNFQNLSVMGLRILLLKVAGFNLLMTLRLWSS |
| RAS TCR-10 | 252 | MGPGLLCWVLLCLLGAGSVETGVTQSPTHLIKTRGQQVTLRCSSQS |
| beta chain | | GHNTVSWYQQALGQGPQFIFQYYREEENGRGNFPPRFSGLQFPNYS |
| | | SELNVNALELDDSALYLCASSTSFWEVNTEAFFGQGTRLTVVKDLR |
| | | NVTPPKVSLFEPSKAEIANKQKATLVCLARGFFPDHVELSWWVNGK |
| | | EVHSGVSTDPQAYKESNYSYCLSSRLRVSATFWHNPRNHFRCQVQF |
| | | HGLSEEDKWPEGSPKPVTQNISAEAWGRADCGITSASYHQGVLSAT |
| | | ILYEILLGKATLYAVLVSGLVLMAMVKKKNS |
| TCR peptide | 253 | VVGACGVGK |
| | 254 | VVVGACGVGK |
| RAS TCR-11 | 255 | TSGFNG |
| alpha chain | | |
| CDR1 | | |
| RAS TCR-11 | 256 | NVLDGL |
| alpha chain | | |
| CDR2 | | |
| RAS TCR-11 | 257 | AVREEVLYNQGGKLI |
| alpha chain | | |
| CDR3 | | |
| RAS TCR-11 | 258 | LNHDA |
| beta chain | | |
| CDR1 | | |
| RAS TCR-11 | 259 | SQIVND |
| beta chain | | |
| CDR2 | | |
| RAS TCR-11 | 260 | ASSKRGWPYEQY |
| beta chain | | |
| CDR3 | | |
| RAS TCR-11 | 261 | ATGTGGGGAGTTTTCCTTCTTTATGTTTCCATGAAGATGGGAGGC |
| alpha chain | | ACTACAGGACAAAACATTGACCAGCCCACTGAGATGACAGCTAC |
| variable | | GGAAGGTGCCATTGTCCAGATCAACTGCACGTACCAGACATCTG |
| domain | | GGTTCAACGGGCTGTTCTGGTACCAGCAACATGCTGGCGAAGCA |
| | | CCCACATTTCTGTCTTACAATGTTCTGGATGGTTTGGAGGAGAAA |
| | | GGTCGTTTTTCTTCATTCCTTAGTCGGTCTAAAGGGTACAGTTACC |
| | | TCCTTTTGAAGGAGCTCCAGATGAAAGACTCTGCCTCTTACCTCT |
| | | GTGCTGTGAGAGAGGAGGTCCTTTATAACCAGGGAGGAAAGCTT |
| | | ATCTTCGGACAGGGAACGGAGTTATCTGTGAAACCC |
| | 262 | ATGTGGGGCGTGTTTCTGCTGTACGTGAGCATGAAGATGGGCGGC |
| | | ACCACAGGCCAGAACATCGACCAGCCAACCGAGATGACCGCCAC |
| | | AGAGGGCGCCATCGTGCAGATCAACTGCACCTACCAGACAAGCG |
| | | GCTTCAATGGCCTGTTTTGGTATCAGCAGCACGCAGGAGAGGCAC |
| | | CCACATTCCTGTCCTATAATGTGCTGGACGGCCTGGAGGAGAAGG |
| | | GCAGGTTCTCCTCTTTTCTGAGCCGCTCCAAGGGCTACTCCTATCT |
| | | GCTGCTGAAGGAGCTGCAGATGAAGGATTCTGCCAGCTACCTGT |
| | | GCGCCGTGCGGGAGGAGGTGCTGTATAATCAGGGCGGCAAGCTG |
| | | ATCTTTGGCCAGGGCACCGAGCTGAGCGTGAAGCCT |
| | 263 | MWGVFLLYVSMKMGGTTGQNIDQPTEMTATEGAIVQINCTYQTSG |
| | | FNGLFWYQQHAGEAPTFLSYNVLDGLEEKGRFSSFLSRSKGYSYL |
| | | LLKELQMKDSASYLCAVREEVLYNQGGKLIFGQGTELSVKP |
| RAS TCR-11 | 264 | ATGAGCAACCAGGTGCTCTGCTGTGTGGTCCTTTGTCTCCTGGGA |
| beta chain | | GCAAACACCGTGGATGGTGGAATCACTCAGTCCCCGAAGTACCT |
| variable | | GTTCAGAAAGGAAGGACAGAATGTGACCCTGAGTTGTGAACAGA |
| domain | | ATTTGAACCACGATGCCATGTACTGGTACCGACAGGACCCAGGG |
| | | CAAGGGCTGAGATTGATCTACTACTCACAGATAGTAAATGACTTT |
| | | CAGAAAGGAGATATAGCTGAAGGGTACAGCGTCTCTCGGGAGAA |
| | | GAAGGAATCCTTTCCTCTCACTGTGACATCGGCCCAAAAGAACCC |
| | | GACAGCTTTCTATCTCTGTGCCAGTAGTAAAAGGGGATGGCCCTA |
| | | CGAGCAGTACTTCGGGCCGGGCACCAGGCTCACGGTCACA |
| | 265 | ATGTCCAACCAGGTGCTGTGCTGCGTGGTGCTGTGCCTGCTGGGA |
| | | GCAAATACCGTGGACGGAGGCATCACACAGTCCCCCAAGTACCT |
| | | GTTCCGGAAGGAGGGCCAGAACGTGACCCTGTCTTGTGAGCAGA |
| | | ACCTGAATCACGACGCCATGTACTGGTATAGGCAGGACCCCGGA |
| | | CAGGGACTGAGACTGATCTACTATAGCCAGATCGTGAACGACTTT |
| | | CAGAAGGGCGACATCGCCGAGGGCTACAGCGTGTCCCGGGAGAA |
| | | GAAGGAGTCCTTCCCACTGACCGTGACATCTGCCCAGAAGAATC |
| | | CCACCGCCTTTTATCTGTGCGCCAGCTCCAAGAGAGGCTGGCCCT |
| | | ACGAGCAGTATTTCGGCCCTGGCACCAGGCTGACCGTGACA |
| | 266 | MSNQVLCCVVLCLLGANTVDGGITQSPKYLFRKEGQNVTLSCEQNL |
| | | NHDAMYWYRQDPGQGLRLIYYSQIVNDFQKGDIAEGYSVSREKKES |
| | | FPLTVTSAQKNPTAFYLCASSKRGWPYEQYFGPGTRLTVT |
| RAS TCR-11 | 267 | MWGVFLLYVSMKMGGTTGQNIDQPTEMTATEGAIVQINCTYQTSG |
| alpha chain | | FNGLFWYQQHAGEAPTFLSYNVLDGLEEKGRFSSFLSRSKGYSYLLL |
| | | KELQMKDSASYLCAVREEVLYNQGGKLIFGQGTELSVKPDIQNPEP |
| | | AVYQLKDPRSQDSTLCLFTDFDSQINVPKTMESGTFITDKTVLDMKA |
| | | MDSKSNGAIAWSNQTSFTCQDIFKETNATYPSSDVPCDATLTEKSFE |
| | | TDMNLNFQNLSVMGLRILLLKVAGFNLLMTLRLWSS |
| RAS TCR-11 | 268 | MSNQVLCCVVLCLLGANTVDGGITQSPKYLFRKEGQNVTLSCEQNL |
| beta chain | | NHDAMYWYRQDPGQGLRLIYYSQIVNDFQKGDIAEGYSVSREKKES |
| | | FPLTVTSAQKNPTAFYLCASSKRGWPYEQYFGPGTRLTVTKDLRNV |
| | | TPPKVSLFEPSKAEIANKQKATLVCLARGFFPDHVELSWWVNGKEV |
| | | HSGVSTDPQAYKESNYSYCLSSRLRVSATFWHNPRNHFRCQVQFHG |
| | | LSEEDKWPEGSPKPVTQNISAEAWGRADCGITSASYHQGVLSATIL |
| | | YEILLGKATLYAVLVSGLVLMAMVKKKNS |
| TCR peptide | 269 | VVGACGVGK |
| | 270 | VVVGACGVGK |
| RAS TCR-12 | 271 | TSINN |
| alpha chain | | |
| CDR1 | | |
| RAS TCR-12 | 272 | IRSNERE |
| alpha chain | | |
| CDR2 | | |
| RAS TCR-12 | 273 | ATDRQSSGDKLT |
| alpha chain | | |
| CDR3 | | |
| RAS TCR-12 | 274 | SGHAT |
| beta chain | | |
| CDR1 | | |
| RAS TCR-12 | 275 | FQNNGV |
| beta chain | | |
| CDR2 | | |
| RAS TCR-12 | 276 | ASSLADIYEQY |
| beta chain | | |
| CDR3 | | |
| RAS TCR-12 | | ATGGAAACTCTCCTGGGAGTGTCTTTGGTGATTCTATGGCTTCAA |
| alpha chain | | CTGGCTAGGGTGAACAGTCAACAGGGAGAAGAGGATCCTCAGGC |
| variable | | CTTGAGCATCCAGGAGGGTGAAAATGCCACCATGAACTGCAGTT |
| domain | | ACAAAACTAGTATAAACAATTTACAGTGGTATAGACAAAATTCA |
| | 277 | GGTAGAGGCCTTGTCCACCTAATTTTAATACGTTCAAATGAAAGA |
| | | GAGAAACACAGTGGAAGATTAAGAGTCACGCTTGACACTTCCAA |
| | | GAAAAGCAGTTCCTTGTTGATCACGGCTTCCCGGGCAGCAGACA |
| | | CTGCTTCTTACTTCTGTGCTACGGACCGTCAAAGCAGCGGAGACA |
| | | AGCTGACTTTTGGGACCGGGACTCGTTTAGCAGTTAGGCCC |
| | 278 | ATGGAGACCCTGCTGGGCGTGTCCCTGGTCATCCTGTGGCTGCAG |
| | | CTGGCCAGGGTGAACAGCCAGCAGGGAGAGGAGGACCCCCAGG |
| | | CCCTGAGCATCCAGGAGGGCGAGAACGCCACCATGAATTGCTCT |
| | | TACAAGACAAGCATCAACAATCTGCAGTGGTATAGGCAGAACTC |
| | | CGGCCGCGGACTGGTGCACCTGATCCTGATCCGGAGCAATGAGA |
| | | GAGAGAAGCACTCCGGCCGGCTGAGAGTGACCCTGGACACATCT |
| | | AAGAAGTCCTCTAGCCTGCTGATCACCGCCTCTCGGGCAGCAGAT |
| | | ACAGCCAGCTACTTCTGTGCCACCGACAGACAGTCCTCTGGCGAT |
| | | AAGCTGACCTTTGGCACCGGCACAAGGCTGGCCGTGCGCCCC |
| | 279 | METLLGVSLVILWLQLARVNSQQGEEDPQALSIQEGENATMNCS |
| | | YKTSINNLQWYRQNSGRGLVHLILIRSNEREKHSGRLRVTLDTS |
| | | KKSSSLLITASRAADTASYFCATDRQSSGDKLTFGTGTRLAVRP |
| RAS TCR-12 | 280 | ATGGGCACCAGGCTCCTCTGCTGGGCGGCCCTCTGTCTCCTGGGA |
| beta chain | | GCAGAACTCACAGAAGCTGGAGTTGCCCAGTCTCCCAGATATAA |
| variable | | GATTATAGAGAAAAGGCAGAGTGTGGCTTTTTGGTGCAATCCTAT |
| domain | | ATCTGGCCATGCTACCCTTTACTGGTACCAGCAGATCCTGGGACA |
| | | GGGCCCAAAGCTTCTGATTCAGTTTCAGAATAACGGTGTAGTGGA |
| | | TGATTCACAGTTGCCTAAGGATCGATTTTCTGCAGAGAGGCTCAA |
| | | AGGAGTAGACTCCACTCTCAAGATCCAGCCTGCAAAGCTTGAGG |
| | | ACTCGGCCGTGTATCTCTGTGCCAGCAGCTTAGCCGACATCTACG |
| | | AGCAGTACTTCGGGCCGGGCACCAGGCTCACGGTCACA |
| | 281 | ATGGGAACAAGGCTGCTGTGCTGGGCCGCCCTGTGCCTGCTGGG |
| | | AGCAGAGCTGACCGAGGCCGGCGTGGCCCAGAGCCCCCGGTACA |
| | | AGATCATCGAGAAGAGACAGAGCGTGGCCTTCTGGTGCAACCCT |
| | | ATCTCCGGCCACGCCACACTGTACTGGTATCAGCAGATCCTGGGC |
| | | CAGGGCCCAAAGCTGCTGATCCAGTTCCAGAACAATGGCGTGGT |
| | | GGACGATTCCCAGCTGCCCAAGGACCGGTTTTCTGCCGAGAGACT |
| | | GAAGGGCGTGGATTCCACCCTGAAGATCCAGCCCGCCAAGCTGG |
| | | AGGACTCTGCCGTGTATCTGTGCGCCAGCTCCCTGGCCGACATCT |
| | | ACGAGCAGTATTTCGGCCCTGGCACAAGGCTGACCGTGACA |
| | 282 | MGTRLLCWAALCLLGAELTEAGVAQSPRYKIIEKRQSVAFWCNPIS |
| | | GHATLYWYQQILGQGPKLLIQFQNNGVVDDSQLPKDRFSAERLKGV |
| | | DSTLKIQPAKLEDSAVYLCASSLADIYEQYFGPGTRLTVT |
| RAS TCR-12 | 283 | METLLGVSLVILWLQLARVNSQQGEEDPQALSIQEGENATMNCSYK |
| alpha chain | | TSINNLQWYRQNSGRGLVHLILIRSNEREKHSGRLRVTLDTSKKSSSL |
| | | LITASRAADTASYFCATDRQSSGDKLTFGTGTRLAVRPDIQNPEPAV |
| | | YQLKDPRSQDSTLCLFTDFDSQINVPKTMESGTFITDKTVLDMKAM |
| | | DSKSNGAIAWSNQTSFTCQDIFKETNATYPSSDVPCDATLTEKSFET |
| | | DMNLNFQNLSVMGLRILLLKVAGFNLLMTLRLWSS |
| RAS TCR-12 | 284 | MGTRLLCWAALCLLGAELTEAGVAQSPRYKIIEKRQSVAFWCNPIS |
| beta chain | | GHATLYWYQQILGQGPKLLIQFQNNGVVDDSQLPKDRFSAERLKGV |
| | | DSTLKIQPAKLEDSAVYLCASSLADIYEQYFGPGTRLTVTKDLRNVT |
| | | PPKVSLFEPSKAEIANKQKATLVCLARGFFPDHVELSWWVNGKEVH |
| | | SGVSTDPQAYKESNYSYCLSSRLRVSATFWHNPRNHFRCQVQFHGL |
| | | SEEDKWPEGSPKPVTQNISAEAWGRADCGITSASYHQGVLSATILYE |
| | | ILLGKATLYAVLVSGLVLMAMVKKKNS |
| TCR peptide | 285 | VVGACGVGK |
| | 286 | VVVGACGVGK |
| RAS TCR-13 | 287 | TSESDYY |
| alpha chain | | |
| CDR1 | | |
| RAS TCR-13 | 288 | QEAYKQQN |
| alpha chain | | |
| CDR2 | | |
| RAS TCR-13 | 289 | ALYIYGGSQGNLI |
| alpha chain | | |
| CDR3 | | |
| RAS TCR-13 | 290 | SEHNR |
| beta chain | | |
| CDR1 | | |
| RAS TCR-13 | 29 | FQNEAQ |
| beta chain | | |
| CDR2 | | |
| RAS TCR-13 | 292 | ASSSTDRIEAF |
| beta chain | | |
| CDR3 | | |
| RAS TCR-13 | 293 | ATGGCATGCCCTGGCTTCCTGTGGGCACTTGTGATCTCCACCTGT |
| alpha chain | | CTTGAATTTAGCATGGCTCAGACAGTCACTCAGTCTCAACCAGAG |
| variable | | ATGTCTGTGCAGGAGGCAGAGACCGTGACCCTGAGCTGCACATA |
| domain | | TGACACCAGTGAGAGTGATTATTATTTATTCTGGTACAAGCAGCC |
| | | TCCCAGCAGGCAGATGATTCTCGTTATTCGCCAAGAAGCTTATAA |
| | | GCAACAGAATGCAACAGAGAATCGTTTCTCTGTGAACTTCCAGA |
| | | AAGCAGCCAAATCCTTCAGTCTCAAGATCTCAGACTCACAGCTGG |
| | | GGGATGCCGCGATGTATTTCTGTGCTCTCTATATTTATGGAGGAA |
| | | GCCAAGGAAATCTCATCTTTGGAAAAGGCACTAAACTCTCTGTTA |
| | | AACCA |
| | 294 | ATGGCATGCCCAGGCTTCCTGTGGGCACTGGTCATCAGCACATGT |
| | | CTGGAGTTTTCTATGGCCCAGACCGTGACACAGTCTCAGCCTGAG |
| | | ATGAGCGTGCAGGAGGCCGAGACCGTGACACTGAGCTGCACCTA |
| | | CGACACATCTGAGAGCGATTACTATCTGTTCTGGTATAAGCAGCC |
| | | ACCCTCCAGACAGATGATCCTGGTCATCAGGCAGGAGGCCTACA |
| | | AGCAGCAGAACGCCACCGAGAATCGGTTCTCCGTGAACTTTCAG |
| | | AAGGCCGCCAAGTCCTTTTCTCTGAAGATCAGCGACTCCCAGCTG |
| | | GGCGATGCCGCCATGTATTTCTGTGCCCTGTACATCTATGGCGGC |
| | | TCTCAGGGCAATCTGATCTTTGGCAAGGGCACCAAGCTGAGCGTG |
| | | AAGCCT |
| | 295 | MACPGFLWALVISTCLEFSMAQTVTQSQPEMSVQEAETVTLSCTYD |
| | | TSESDYYLFWYKQPPSRQMILVIRQEAYKQQNATENRFSVNFQKAA |
| | | KSFSLKISDSQLGDAAMYFCALYIYGGSQGNLIFGKGTKLSVKP |
| RAS TCR-13 | 296 | ATGGGCACCAGCCTCCTCTGCTGGATGGCCCTGTGTCTCCTGGGG |
| beta chain | | GCAGATCACGCAGATACTGGAGTCTCCCAGGACCCCAGACACAA |
| variable | | GATCACAAAGAGGGGACAGAATGTAACTTTCAGGTGTGATCCAA |
| domain | | TTTCTGAACACAACCGCCTTTATTGGTACCGACAGACCCTGGGGC |
| | | AGGGCCCAGAGTTTCTGACTTACTTCCAGAATGAAGCTCAACTAG |
| | | AAAAATCAAGGCTGCTCAGTGATCGGTTCTCTGCAGAGAGGCCT |
| | | AAGGGATCTTTCTCCACCTTGGAGATCCAGCGCACAGAGCAGGG |
| | | GGACTCGGCCATGTATCTCTGTGCCAGCAGCTCCACCGACAGGAT |
| | | TGAAGCTTTCTTTGGACAAGGCACCAGACTCACAGTTGTA |
| | 297 | ATGGGCACCTCCCTGCTGTGCTGGATGGCACTGTGCCTGCTGGGA |
| | | GCAGACCACGCAGATACAGGCGTGTCTCAGGACCCACGCCACAA |
| | | GATCACCAAGCGGGGCCAGAACGTGACATTCAGATGCGATCCCA |
| | | TCTCCGAGCACAATAGGCTGTACTGGTATAGGCAGACCCTGGGA |
| | | CAGGGACCAGAGTTCCTGACATACTTTCAGAACGAGGCCCAGCT |
| | | GGAGAAGAGCCGGCTGCTGTCCGACAGATTCTCTGCCGAGAGGC |
| | | CCAAGGGCTCTTTTAGCACCCTGGAGATCCAGAGAACAGAGCAG |
| | | GGCGACAGCGCCATGTATCTGTGCGCCAGCTCCTCTACCGATAGG |
| | | ATCGAGGCCTTCTTTGGCCAGGGCACCCGCCTGACAGTGGTG |
| | 298 | MGTSLLCWMALCLLGADHADTGVSQDPRHKITKRGQNVTFRCDPIS |
| | | EHNRLYWYRQTLGQGPEFLTYFQNEAQLEKSRLLSDRFSAERPKGS |
| | | FSTLEIQRTEQGDSAMYLCASSSTDRIEAFFGQGTRLTVV |
| RAS TCR-13 | 299 | MACPGFLWALVISTCLEFSMAQTVTQSQPEMSVQEAETVTLSCTYD |
| alpha chain | | TSESDYYLFWYKQPPSRQMILVIRQEAYKQQNATENRFSVNFQKAA |
| | | KSFSLKISDSQLGDAAMYFCALYIYGGSQGNLIFGKGTKLSVKPDIQ |
| | | NPEPAVYQLKDPRSQDSTLCLFTDFDSQINVPKTMESGTFITDKTVL |
| | | DMKAMDSKSNGAIAWSNQTSFTCQDIFKETNATYPSSDVPCDATLT |
| | | EKSFETDMNLNFQNLSVMGLRILLLKVAGFNLLMTLRLWSS |
| RAS TCR-13 | 300 | MGTSLLCWMALCLLGADHADTGVSQDPRHKITKRGQNVTFRCDPIS |
| beta chain | | EHNRLYWYRQTLGQGPEFLTYFQNEAQLEKSRLLSDRFSAERPKGS |
| | | FSTLEIQRTEQGDSAMYLCASSSTDRIEAFFGQGTRLTVVKDLRNVT |
| | | PPKVSLFEPSKAEIANKQKATLVCLARGFFPDHVELSWWVNGKEVH |
| | | SGVSTDPQAYKESNYSYCLSSRLRVSATFWHNPRNHFRCQVQFHGL |
| | | SEEDKWPEGSPKPVTQNISAEAWGRADCGITSASYHQGVLSATILYE |
| | | ILLGKATLYAVLVSGLVLMAMVKKKNS |
| TCR | 301 | VVGADGVGK |
| Peptides | 302 | VVVGADGVGK |
| RAS TCR-14 | 303 | VSNAYN |
| alpha chain | | |
| CDR1 | | |
| RAS TCR-14 | 304 | GSKP |
| alpha chain | | |
| CDR2 | | |
| RAS TCR-14 | 305 | ATYNFNKFY |
| alpha chain | | |
| CDR3 | | |
| RAS TCR-14 | 306 | SGHTS |
| beta chain | | |
| CDR1 | | |
| RAS TCR-14 | 307 | YDEGEE |
| beta chain | | |
| CDR2 | | |
| RAS TCR-14 | 308 | ASTTFKTGRAIEKLF |
| beta chain | | |
| CDR3 | | |
| RAS TCR-14 | 309 | ATGGCTTTGCAGAGCACTCTGGGGGCGGTGTGGCTAGGGCTTCTC |
| alpha chain | | CTCAACTCTCTCTGGAAGGTTGCAGAAAGCAAGGACCAAGTGTTT |
| variable | | CAGCCTTCCACAGTGGCATCTTCAGAGGGAGCTGTGGTGGAAATC |
| domain | | TTCTGTAATCACTCTGTGTCCAATGCTTACAACTTCTTCTGGTACC |
| | | TTCACTTCCCGGGATGTGCACCAAGACTCCTTGTTAAAGGCTCAA |
| | | AGCCTTCTCAGCAGGGACGATACAACATGACCTATGAACGGTTCT |
| | | CTTCATCGCTGCTCATCCTCCAGGTGCGGGAGGCAGATGCTGCTG |
| | | TTTACTACTGTGCTACGTACAACTTCAACAAATTTTACTTTGGATC |
| | | TGGGACCAAACTCAATGTAAAACCA |
| | 310 | ATGGCCCTGCAGTCTACACTGGGAGCCGTGTGGCTGGGACTGCTG |
| | | CTGAACTCTCTGTGGAAGGTGGCCGAGAGCAAGGACCAGGTGTT |
| | | CCAGCCTAGCACCGTGGCCTCCTCTGAGGGAGCAGTGGTGGAGA |
| | | TCTTTTGCAATCACTCCGTGTCTAACGCCTACAATTTCTTTTGGTA |
| | | TCTGCACTTTCCAGGATGTGCACCAAGGCTGCTGGTGAAGGGCAG |
| | | CAAGCCATCCCAGCAGGGCCGGTACAACATGACCTATGAGAGAT |
| | | TCAGCTCCTCTCTGCTGATCCTGCAGGTGAGAGAGGCCGATGCCG |
| | | CCGTGTACTATTGTGCCACCTACAACTTTAATAAGTTCTATTTTGG |
| | | CTCCGGCACAAAGCTGAATGTGAAGCCT |
| | 311 | MALQSTLGAVWLGLLLNSLWKVAESKDQVFQPSTVASSEGAVVEIF |
| | | CNHSVSNAYNFFWYLHFPGCAPRLLVKGSKPSQQGRYNMTYERFSS |
| | | SLLILQVREADAAVYYCATYNFNKFYFGSGTKLNVKP |
| RAS TCR-14 | 312 | ATGGGACCCAGGCTCCTCTTCTGGGCACTGCTTTGTCTCCTCGGA |
| beta chain | | ACAGGCCCAGTGGAGGCTGGAGTCACACAAAGTCCCACACACCT |
| variable | | GATCAAAACGAGAGGACAGCAAGCGACTCTGAGATGCTCTCCTA |
| domain | | TCTCTGGGCACACCAGTGTGTACTGGTACCAACAGGCCCTGGGTC |
| | | TGGGCCTCCAGTTCCTCCTTTGGTATGACGAGGGTGAAGAGAGAA |
| | | ACAGAGGAAACTTCCCTCCTAGATTTTCAGGTCGCCAGTTCCCTA |
| | | ATTATAGCTCTGAGCTGAATGTGAACGCCTTGGAGCTGGAGGACT |
| | | CGGCCCTGTATCTCTGTGCCAGCACCACTTTTAAGACGGGACGGG |
| | | CAATTGAAAAACTGTTTTTTGGCAGTGGAACCCAGCTCTCTGTCT |
| | | TG |
| | 313 | ATGGGACCAAGGCTGCTGTTCTGGGCACTGCTGTGCCTGCTGGGA |
| | | ACCGGACCTGTGGAGGCCGGCGTGACCCAGTCTCCAACACACCT |
| | | GATCAAGACCAGGGGACAGCAGGCCACACTGAGGTGTAGCCCCA |
| | | TCTCCGGCCACACAAGCGTGTACTGGTATCAGCAGGCCCTGGGA |
| | | CTGGGACTGCAGTTCCTGCTGTGGTACGACGAGGGCGAGGAGAG |
| | | GAACCGCGGCAATTTCCCACCTCGGTTCAGCGGCCGGCAGTTTCC |
| | | CAACTACAGCTCCGAGCTGAACGTGAATGCCCTGGAGCTGGAGG |
| | | ACAGCGCCCTGTATCTGTGCGCCTCCACCACATTCAAGACCGGCA |
| | | GGGCCATCGAGAAGCTGTTCTTTGGCTCTGGCACCCAGCTGAGCG |
| | | TGCTG |
| | 314 | MGPRLLFWALLCLLGTGPVEAGVTQSPTHLIKTRGQQATLRCSPISG |
| | | HTSVYWYQQALGLGLQFLLWYDEGEERNRGNFPPRFSGRQFPNYSS |
| | | ELNVNALELEDSALYLCASTTFKTGRAIEKLFFGSGTQLSVL |
| RAS TCR-14 | 315 | MALQSTLGAVWLGLLLNSLWKVAESKDQVFQPSTVASSEGAVVEIF |
| alpha chain | | CNHSVSNAYNFFWYLHFPGCAPRLLVKGSKPSQQGRYNMTYERFSS |
| | | SLLILQVREADAAVYYCATYNFNKFYFGSGTKLNVKPDIQNPEPAV |
| | | YQLKDPRSQDSTLCLFTDFDSQINVPKTMESGTFITDKTVLDMKAM |
| | | DSKSNGAIAWSNQTSFTCQDIFKETNATYPSSDVPCDATLTEKSFET |
| | | DMNLNFQNLSVMGLRILLLKVAGFNLLMTLRLWSS |
| RAS TCR-14 | 316 | MGPRLLFWALLCLLGTGPVEAGVTQSPTHLIKTRGQQATLRCSPISG |
| beta chain | | HTSVYWYQQALGLGLQFLLWYDEGEERNRGNFPPRFSGRQFPNYSS |
| | | ELNVNALELEDSALYLCASTTFKTGRAIEKLFFGSGTQLSVLKDLRN |
| | | VTPPKVSLFEPSKAEIANKQKATLVCLARGFFPDHVELSWWVNGKE |
| | | VHSGVSTDPQAYKESNYSYCLSSRLRVSATFWHNPRNHFRCQVQFH |
| | | GLSEEDKWPEGSPKPVTQNISAEAWGRADCGITSASYHQGVLSATIL |
| | | YEILLGKATLYAVLVSGLVLMAMVKKKNS |
| TCR peptide | 317 | VVGADGVGK |
| | 318 | VVVGADGVGK |
| RAS TCR-15 | 319 | TSGFNG |
| alpha chain | | |
| CDR1 | | |
| RAS TCR-15 | 320 | NVLDGL |
| alpha chain | | |
| CDR2 | | |
| RAS TCR-15 | 32 | AVRDRGGSYIPT |
| alpha chain | | |
| CDR3 | | |
| RAS TCR-15 | 322 | MNHEY |
| beta chain | | |
| CDR1 | | |
| RAS TCR-15 | 323 | SMNVEV |
| beta chain | | |
| CDR2 | | |
| RAS TCR-15 | 324 | ASSSRGHSGTEAF |
| beta chain | | |
| CDR3 | | |
| RAS TCR-15 | 325 | ATGTGGGGAGTTTTCCTTCTTTATGTTTCCATGAAGATGGGAGGC |
| alpha chain | | ACTACAGGACAAAACATTGACCAGCCCACTGAGATGACAGCTAC |
| variable | | GGAAGGTGCCATTGTCCAGATCAACTGCACGTACCAGACATCTG |
| domain | | GGTTCAACGGGCTGTTCTGGTACCAGCAACATGCTGGCGAAGCA |
| | | CCCACATTTCTGTCTTACAATGTTCTGGATGGTTTGGAGGAGAAA |
| | | GGTCGTTTTTCTTCATTCCTTAGTCGGTCTAAAGGGTACAGTTACC |
| | | TCCTTTTGAAGGAGCTCCAGATGAAAGACTCTGCCTCTTACCTCT |
| | | GTGCTGTGAGAGATCGAGGAGGAAGCTACATACCTACATTTGGA |
| | | AGAGGAACCAGCCTTATTGTTCATCCG |
| | 326 | ATGTGGGGCGTGTTTCTGCTGTACGTGTCTATGAAGATGGGCGGC |
| | | ACCACAGGCCAGAACATCGACCAGCCTACCGAGATGACCGCCAC |
| | | AGAGGGCGCCATCGTGCAGATCAACTGCACCTACCAGACATCTG |
| | | GCTTCAATGGCCTGTTTTGGTATCAGCAGCACGCCGGCGAGGCCC |
| | | CAACATTCCTGTCCTATAATGTGCTGGATGGCCTGGAGGAGAAGG |
| | | GCAGGTTCTCTAGCTTTCTGTCCCGCTCTAAGGGCTACAGCTATCT |
| | | GCTGCTGAAGGAGCTGCAGATGAAGGACAGCGCCTCCTACCTGT |
| | | GCGCCGTGCGGGATAGAGGAGGCTCCTATATCCCTACCTTTGGCC |
| | | GGGGCACATCTCTGATCGTGCACCCA |
| | 327 | MWGVFLLYVSMKMGGTTGQNIDQPTEMTATEGAIVQINCTYQTSG |
| | | FNGLFWYQQHAGEAPTFLSYNVLDGLEEKGRFSSFLSRSKGYSYL |
| | | LLKELQMKDSASYLCAVRDRGGSYIPTFGRGTSLIVHP |
| RAS TCR-15 | 328 | ATGGGCCCCCAGCTCCTTGGCTATGTGGTCCTTTGCCTTCTAGGA |
| beta chain | | GCAGGCCCCCTGGAAGCCCAAGTGACCCAGAACCCAAGATACCT |
| variable | | CATCACAGTGACTGGAAAGAAGTTAACAGTGACTTGTTCTCAGA |
| domain | | ATATGAACCATGAGTATATGTCCTGGTATCGACAAGACCCAGGG |
| | | CTGGGCTTAAGGCAGATCTACTATTCAATGAATGTTGAGGTGACT |
| | | GATAAGGGAGATGTTCCTGAAGGGTACAAAGTCTCTCGAAAAGA |
| | | GAAGAGGAATTTCCCCCTGATCCTGGAGTCGCCCAGCCCCAACC |
| | | AGACCTCTCTGTACTTCTGTGCCAGCAGTTCCAGGGGGCATTCGG |
| | | GCACTGAAGCTTTCTTTGGACAAGGCACCAGACTCACAGTTGTA |
| | 329 | ATGGGACCACAGCTGCTGGGATACGTGGTGCTGTGCCTGCTGGG |
| | | AGCAGGACCACTGGAGGCACAGGTGACCCAGAACCCACGGTATC |
| | | TGATCACCGTGACAGGCAAGAAGCTGACCGTGACATGTTCTCAG |
| | | AACATGAATCACGAGTACATGAGCTGGTATAGGCAGGACCCTGG |
| | | ACTGGGACTGAGACAGATCTACTATAGCATGAATGTGGAGGTGA |
| | | CCGACAAGGGCGATGTGCCCGAGGGCTACAAGGTGTCCAGGAAG |
| | | GAGAAGCGCAACTTCCCTCTGATCCTGGAGTCCCCATCTCCCAAT |
| | | CAGACCAGCCTGTATTTTTGCGCCAGCTCCTCTAGGGGACACTCC |
| | | GGAACAGAGGCCTTCTTTGGCCAGGGCACCAGGCTGACAGTGGT |
| | | G |
| | 330 | MGPQLLGYVVLCLLGAGPLEAQVTQNPRYLITVTGKKLTVTCSQNM |
| | | NHEYMSWYRQDPGLGLRQIYYSMNVEVTDKGDVPEGYKVSRKEK |
| | | RNFPLILESPSPNQTSLYFCASSSRGHSGTEAFFGQGTRLTVV |
| RAS TCR-15 | 331 | MWGVFLLYVSMKMGGTTGQNIDQPTEMTATEGAIVQINCTYQTSG |
| alpha chain | | FNGLFWYQQHAGEAPTFLSYNVLDGLEEKGRFSSFLSRSKGYSYLLL |
| | | KELQMKDSASYLCAVRDRGGSYIPTFGRGTSLIVHPDIQNPEPAVYQ |
| | | LKDPRSQDSTLCLFTDFDSQINVPKTMESGTFITDKTVLDMKAMDSK |
| | | SNGAIAWSNQTSFTCQDIFKETNATYPSSDVPCDATLTEKSFETDMN |
| | | LNFQNLSVMGLRILLLKVAGFNLLMTLRLWSS |
| RAS TCR-15 | 332 | MGPQLLGYVVLCLLGAGPLEAQVTQNPRYLITVTGKKLTVTCSQNM |
| beta chain | | NHEYMSWYRQDPGLGLRQIYYSMNVEVTDKGDVPEGYKVSRKEKRN |
| | | FPLILESPSPNQTSLYFCASSSRGHSGTEAFFGQGTRLTVVKDLRN |
| | | VTPPKVSLFEPSKAEIANKQKATLVCLARGFFPDHVELSWWVNGKE |
| | | VHSGVSTDPQAYKESNYSYCLSSRLRVSATFWHNPRNHFRCQVQFH |
| | | GLSEEDKWPEGSPKPVTQNISAEAWGRADCGITSASYHQGVLSATI |
| | | LYEILLGKATLYAVLVSGLVLMAMVKKKNS |
| TCR | 333 | VVGADGVGK |
| Peptides | 334 | VVVGADGVGK |
| RAS TCR-16 | 335 | TSINN |
| alpha chain | | |
| CDR1 | | |
| RAS TCR-16 | 336 | IRSNERE |
| alpha chain | | |
| CDR2 | | |
| RAS TCR-16 | 337 | AGLYSSASKII |
| alpha chain | | |
| CDR3 | | |
| RAS TCR-16 | 338 | MNHEY |
| beta chain | | |
| CDR1 | | |
| RAS TCR-16 | 339 | SMNVEV |
| beta chain | | |
| CDR2 | | |
| RAS TCR-16 | 340 | ASSSRGHSGTEAF |
| beta chain | | |
| CDR3 | | |
| RAS TCR-16 | 341 | ATGGAAACTCTCCTGGGAGTGTCTTTGGTGATTCTATGGCTTCAA |
| alpha chain | | CTGGCTAGGGTGAACAGTCAACAGGGAGAAGAGGATCCTCAGGC |
| variable | | CTTGAGCATCCAGGAGGGTGAAAATGCCACCATGAACTGCAGTT |
| domain | | ACAAAACTAGTATAAACAATTTACAGTGGTATAGACAAAATTCA |
| | | GGTAGAGGCCTTGTCCACCTAATTTTAATACGTTCAAATGAAAGA |
| | | GAGAAACACAGTGGAAGATTAAGAGTCACGCTTGACACTTCCAA |
| | | GAAAAGCAGTTCCTTGTTGATCACGGCTTCCCGGGCAGCAGACA |
| | | CTGCTTCTTACTTCTGTGCTGGGCTGTACAGCAGTGCTTCCAAGAT |
| | | AATCTTTGGATCAGGGACCAGACTCAGCATCCGGCCA |
| | 342 | ATGGAGACACTGCTGGGCGTGTCCCTGGTCATCCTGTGGCTGCAG |
| | | CTGGCCAGAGTGAACAGCCAGCAGGGAGAGGAGGACCCTCAGG |
| | | CCCTGAGCATCCAGGAGGGCGAGAACGCCACCATGAATTGCTCT |
| | | TACAAGACAAGCATCAACAATCTGCAGTGGTATAGGCAGAACTC |
| | | CGGCCGCGGACTGGTGCACCTGATCCTGATCAGGTCTAATGAGC |
| | | GCGAGAAGCACAGCGGCCGGCTGAGAGTGACCCTGGACACAAGC |
| | | AAGAAGTCTAGCTCCCTGCTGATCACCGCCTCCAGAGCAGCAGA |
| | | TACAGCCTCTTACTTCTGTGCCGGCCTGTATTCTAGCGCCTCCAA |
| | | GATCATCTTTGGCAGCGGCACCCGGCTGTCCATCAGACCC |
| | 343 | METLLGVSLVILWLQLARVNSQQGEEDPQALSIQEGENATMNCS |
| | | YKTSINNLQWYRQNSGRGLVHLILIRSNEREKHSGRLRVTLDTS |
| | | KKSSSLLITASRAADTASYFCAGLYSSASKIIFGSGTRLSIRP |
| RAS TCR-16 | 344 | ATGGGCCCCCAGCTCCTTGGCTATGTGGTCCTTTGCCTTCTAGGA |
| beta chain | | GCAGGCCCCCTGGAAGCCCAAGTGACCCAGAACCCAAGATACCT |
| variable | | CATCACAGTGACTGGAAAGAAGTTAACAGTGACTTGTTCTCAGA |
| domain | | ATATGAACCATGAGTATATGTCCTGGTATCGACAAGACCCAGGG |
| | | CTGGGCTTAAGGCAGATCTACTATTCAATGAATGTTGAGGTGACT |
| | | GATAAGGGAGATGTTCCTGAAGGGTACAAAGTCTCTCGAAAAGA |
| | | GAAGAGGAATTTCCCCCTGATCCTGGAGTCGCCCAGCCCCAACC |
| | | AGACCTCTCTGTACTTCTGTGCCAGCAGTTCCAGGGGGCATTCGG |
| | | GCACTGAAGCTTTCTTTGGACAAGGCACCAGACTCACAGTTGTA |
| | 345 | ATGGGCCCACAGCTGCTGGGCTACGTGGTGCTGTGCCTGCTGGGA |
| | | GCAGGACCACTGGAGGCACAGGTGACCCAGAACCCCAGGTATCT |
| | | GATCACCGTGACAGGCAAGAAGCTGACCGTGACATGTAGCCAGA |
| | | ACATGAATCACGAGTACATGTCCTGGTATAGGCAGGACCCCGGA |
| | | CTGGGACTGAGACAGATCTACTATTCCATGAATGTGGAGGTGACC |
| | | GACAAGGGCGATGTGCCTGAGGGCTACAAGGTGTCTAGGAAGGA |
| | | GAAGCGCAACTTCCCACTGATCCTGGAGTCCCCATCTCCCAATCA |
| | | GACCTCCCTGTATTTTTGCGCCAGCTCCTCTAGGGGCCACTCTGG |
| | | CACAGAGGCCTTCTTTGGCCAGGGCACCAGGCTGACAGTGGTG |
| | 346 | MGPQLLGYVVLCLLGAGPLEAQVTQNPRYLITVTGKKLTVTCSQNM |
| | | NHEYMSWYRQDPGLGLRQIYYSMNVEVTDKGDVPEGYKVSRKEK |
| | | RNFPLILESPSPNQTSLYFCASSSRGHSGTEAFFGQGTRLTVV |
| RAS TCR-16 | 347 | METLLGVSLVILWLQLARVNSQQGEEDPQALSIQEGENATMNCSYK |
| alpha chain | | TSINNLQWYRQNSGRGLVHLILIRSNEREKHSGRLRVTLDTSKKSS |
| | | SLLITASRAADTASYFCAGLYSSASKIIFGSGTRLSIRPDIQNPEP |
| | | AVYQLKDPRSQDSTLCLFTDFDSQINVPKTMESGTFITDKTVLDMK |
| | | AMDSKSNGAIAWSNQTSFTCQDIFKETNATYPSSDVPCDATLTEKS |
| | | FETDMNLNFQNLSVMGLRILLLKVAGFNLLMTLRLWSS |
| RAS TCR-16 | 348 | MGPQLLGYVVLCLLGAGPLEAQVTQNPRYLITVTGKKLTVTCSQNM |
| beta chain | | NHEYMSWYRQDPGLGLRQIYYSMNVEVTDKGDVPEGYKVSRKEKRN |
| | | FPLILESPSPNQTSLYFCASSSRGHSGTEAFFGQGTRLTVVKDLRN |
| | | VTPPKVSLFEPSKAEIANKQKATLVCLARGFFPDHVELSWWVNGKE |
| | | VHSGVSTDPQAYKESNYSYCLSSRLRVSATFWHNPRNHFRCQVQFH |
| | | GLSEEDKWPEGSPKPVTQNISAEAWGRADCGITSASYHQGVLSATI |
| | | LYEILLGKATLYAVLVSGLVLMAMVKKKNS |
| TCR | 349 | VVGADGVGK |
| Peptides | 350 | VVVGADGVGK |
| RAS TCR-17 | 351 | NIATNDY |
| alpha chain | | |
| CDR1 | | |
| RAS TCR-17 | 352 | GYKTK |
| alpha chain | | |
| CDR2 | | |
| RAS TCR-17 | 353 | LANTGGFKTI |
| alpha chain | | |
| CDR3 | | |
| RAS TCR-17 | 354 | SGHVS |
| beta chain | | |
| CDR1 | | |
| RAS TCR-17 | 355 | FQNEAQ |
| beta chain | | |
| CDR2 | | |
| RAS TCR-17 | 356 | ATYKVGDEQF |
| beta chain | | |
| CDR3 | | |
| RAS TCR-17 | 357 | ATGAGGCAAGTGGCGAGAGTGATCGTGTTCCTGACCCTGAGTACT |
| alpha chain | | TTGAGCCTTGCTAAGACCACCCAGCCCATCTCCATGGACTCATAT |
| variable | | GAAGGACAAGAAGTGAACATAACCTGTAGCCACAACAACATTGC |
| domain | | TACAAATGATTATATCACGTGGTACCAACAGTTTCCCAGCCAAGG |
| | | ACCACGATTTATTATTCAAGGATACAAGACAAAAGTTACAAACG |
| | | AAGTGGCCTCCCTGTTTATCCCTGCCGACAGAAAGTCCAGCACTC |
| | | TGAGCCTGCCCCGGGTTTCCCTGAGCGACACTGCTGTGTACTACT |
| | | GCCTCGCTAATACTGGAGGCTTCAAAACTATCTTTGGAGCAGGAA |
| | | CAAGACTATTTGTTAAAGCA |
| | 358 | ATGAGGCAGGTGGCACGCGTGATCGTGTTTCTGACCCTGAGCACA |
| | | CTGTCCCTGGCCAAGACCACACAGCCTATCTCTATGGACAGCTAC |
| | | GAGGGCCAGGAGGTGAACATCACCTGCTCTCACAACAATATCGC |
| | | CACCAATGATTACATCACATGGTATCAGCAGTTCCCCAGCCAGGG |
| | | CCCTCGGTTTATCATCCAGGGCTATAAGACCAAGGTGACAAACG |
| | | AGGTGGCCAGCCTGTTCATCCCTGCCGACAGGAAGTCTAGCACCC |
| | | TGTCCCTGCCACGCGTGAGCCTGTCCGATACAGCCGTGTACTATT |
| | | GTCTGGCCAATACCGGCGGCTTCAAGACAATCTTTGGCGCCGGCA |
| | | CCAGACTGTTTGTGAAGGCC |
| | 359 | MRQVARVIVELTLSTLSLAKTTQPISMDSYEGQEVNITCSHNNIA |
| | | TNDYITWYQQFPSQGPRFIIQGYKTKVTNEVASLFIPADRKSSTL |
| | | SLPRVSLSDTAVYYCLANTGGFKTIFGAGTRLFVKA |
| RAS TCR-17 | 360 | ATGGGCACCAGGCTCCTCTGCTGGGTGGTCCTGGGTTTCCTAGGG |
| beta chain | | ACAGATCACACAGGTGCTGGAGTCTCCCAGTCCCCTAGGTACAA |
| variable | | AGTCGCAAAGAGAGGACAGGATGTAGCTCTCAGGTGTGATCCAA |
| domains | | TTTCGGGTCATGTATCCCTTTTTTGGTACCAACAGGCCCTGGGGC |
| | | AGGGGCCAGAGTTTCTGACTTATTTCCAGAATGAAGCTCAACTAG |
| | | ACAAATCGGGGCTGCCCAGTGATCGCTTCTTTGCAGAAAGGCCTG |
| | | AGGGATCCGTCTCCACTCTGAAGATCCAGCGCACACAGCAGGAG |
| | | GACTCCGCCGTGTATCTCTGTGCCACCTATAAGGTCGGGGATGAG |
| | | CAGTTCTTCGGGCCAGGGACACGGCTCACCGTGCTA |
| | 361 | ATGGGAACCAGGCTGCTGTGCTGGGTGGTGCTGGGCTTCCTGGGA |
| | | ACCGACCACACAGGAGCAGGCGTGTCCCAGTCTCCAAGGTACAA |
| | | GGTGGCAAAGAGGGGACAGGACGTGGCCCTGAGATGTGATCCTA |
| | | TCTCCGGCCACGTGTCTCTGTTTTGGTACCAGCAGGCCCTGGGAC |
| | | AGGGACCTGAGTTCCTGACCTATTTTCAGAACGAGGCACAGCTGG |
| | | ACAAGAGCGGACTGCCATCCGATCGGTTCTTTGCAGAGAGACCA |
| | | GAGGGCAGCGTGTCCACCCTGAAGATCCAGAGGACACAGCAGGA |
| | | GGACTCCGCCGTGTACCTGTGCGCCACATATAAAGTGGGCGATG |
| | | AGCAGTTCTTTGGCCCAGGCACCCGGCTGACAGTGCTG |
| | 362 | MGTRLLCWVVLGFLGTDHTGAGVSQSPRYKVAKRGQDVALRCDPI |
| | | SGHVSLFWYQQALGQGPEFLTYFQNEAQLDKSGLPSDRFFAERPEG |
| | | SVSTLKIQRTQQEDSAVYLCATYKVGDEQFFGPGTRLTVL |
| RAS TCR-17 | 363 | MRQVARVIVELTLSTLSLAKTTQPISMDSYEGQEVNITCSHNNIATND |
| alpha chain | | YITWYQQFPSQGPRFIIQGYKTKVTNEVASLFIPADRKSSTLSLPRVSL |
| | | SDTAVYYCLANTGGFKTIFGAGTRLFVKADIQNPEPAVYQLKDPRS |
| | | QDSTLCLFTDFDSQINVPKTMESGTFITDKTVLDMKAMDSKSNGAIA |
| | | WSNQTSFTCQDIFKETNATYPSSDVPCDATLTEKSFETDMNLNFQNL |
| | | SVMGLRILLLKVAGFNLLMTLRLWSS |
| RAS TCR-17 | 364 | MGTRLLCWVVLGFLGTDHTGAGVSQSPRYKVAKRGQDVALRCDPI |
| beta chain | | SGHVSLFWYQQALGQGPEFLTYFQNEAQLDKSGLPSDRFFAERPEG |
| | | SVSTLKIQRTQQEDSAVYLCATYKVGDEQFFGPGTRLTVLKDLRNV |
| | | TPPKVSLFEPSKAEIANKQKATLVCLARGFFPDHVELSWWVNGKEV |
| | | HSGVSTDPQAYKESNYSYCLSSRLRVSATFWHNPRNHFRCQVQFHG |
| | | LSEEDKWPEGSPKPVTQNISAEAWGRADCGITSASYHQGVLSATILY |
| | | EILLGKATLYAVLVSGLVLMAMVKKKNS |
| TCR | 365 | VVGADGVGK |
| Peptides | 366 | VVVGADGVGK |
| RAS TCR-18 | 367 | DSASNY |
| alpha chain | | |
| CDR1 | | |
| RAS TCR-18 | 368 | IRSNVGE |
| alpha chain | | |
| CDR2 | | |
| RAS TCR-18 | 369 | AETGFQKLV |
| alpha chain | | |
| CDR3 | | |
| RAS TCR-18 | 370 | MDHEN |
| beta chain | | |
| CDR1 | | |
| RAS TCR-18 | 371 | SYDVKM |
| beta chain | | |
| CDR2 | | |
| RAS TCR-18 | 372 | ASSDWLAGAKDEQY |
| beta chain | | |
| CDR3 | | |
| RAS TCR-18 | 373 | ATGACATCCATTCGAGCTGTATTTATATTCCTGTGGCTGCAGCTG |
| alpha chain | | GACTTGGTGAATGGAGAGAATGTGGAGCAGCATCCTTCAACCCT |
| variable | | GAGTGTCCAGGAGGGAGACAGCGCTGTTATCAAGTGTACTTATTC |
| domains | | AGACAGTGCCTCAAACTACTTCCCTTGGTATAAGCAAGAACTTGG |
| | | AAAAAGACCTCAGCTTATTATAGACATTCGTTCAAATGTGGGCGA |
| | | AAAGAAAGACCAACGAATTGCTGTTACATTGAACAAGACAGCCA |
| | | AACATTTCTCCCTGCACATCACAGAGACCCAACCTGAAGACTCGG |
| | | CTGTCTACTTCTGTGCAGAAACAGGCTTTCAGAAACTTGTATTTG |
| | | GAACTGGCACCCGACTTCTGGTCAGTCCA |
| | 374 | ATGACATCTATCCGCGCCGTGTTCATCTTTCTGTGGCTGCAGCTG |
| | | GACCTGGTGAACGGCGAGAATGTGGAGCAGCACCCAAGCACCCT |
| | | GTCCGTGCAGGAGGGCGACAGCGCCGTGATCAAGTGCACATACT |
| | | CTGATAGCGCCTCCAACTACTTTCCCTGGTATAAGCAGGAGCTGG |
| | | GCAAGCGGCCTCAGCTGATCATCGACATCAGATCCAACGTGGGC |
| | | GAGAAGAAGGATCAGCGGATCGCCGTGACCCTGAATAAGACAGC |
| | | CAAGCACTTCAGCCTGCACATCACCGAGACACAGCCCGAGGATT |
| | | CCGCCGTGTATTTTTGTGCCGAGACCGGCTTCCAGAAGCTGGTGT |
| | | TTGGCACCGGCACAAGACTGCTGGTGTCCCCT |
| | 375 | MTSIRAVFIFLWLQLDLVNGENVEQHPSTLSVQEGDSAVIKCTYSDS |
| | | ASNYFPWYKQELGKRPQLIIDIRSNVGEKKDQRIAVTLNKTAKHFSL |
| | | HITETQPEDSAVYFCAETGFQKLVFGTGTRLLVSP |
| RAS TCR-18 | 376 | ATGGGAATCAGGCTCCTCTGTCGTGTGGCCTTTTGTTTCCTGGCTG |
| beta chain | | TAGGCCTCGTAGATGTGAAAGTAACCCAGAGCTCGAGATATCTA |
| variable | | GTCAAAAGGACGGGAGAGAAAGTTTTTCTGGAATGTGTCCAGGA |
| domains | | TATGGACCATGAAAATATGTTCTGGTATCGACAAGACCCAGGTCT |
| | | GGGGCTACGGCTGATCTATTTCTCATATGATGTTAAAATGAAAGA |
| | | AAAAGGAGATATTCCTGAGGGGTACAGTGTCTCTAGAGAGAAGA |
| | | AGGAGCGCTTCTCCCTGATTCTGGAGTCCGCCAGCACCAACCAGA |
| | | CATCTATGTACCTCTGTGCCAGCAGTGACTGGCTAGCGGGAGCGA |
| | | AGGACGAGCAGTACTTCGGGCCGGGCACCAGGCTCACGGTCACA |
| | 377 | ATGGGCATCCGGCTGCTGTGCAGAGTGGCCTTCTGTTTTCTGGCC |
| | | GTGGGCCTGGTGGACGTGAAGGTGACCCAGAGCTCCCGGTACCT |
| | | GGTGAAGAGAACAGGCGAGAAGGTGTTCCTGGAGTGCGTGCAGG |
| | | ACATGGATCACGAGAACATGTTTTGGTATAGGCAGGACCCCGGA |
| | | CTGGGACTGAGACTGATCTACTTCAGCTATGACGTGAAGATGAA |
| | | GGAGAAGGGCGACATCCCAGAGGGCTACAGCGTGTCCAGGGAG |
| | | AAGAAGGAGCGGTTCAGCCTGATCCTGGAGTCTGCCAGCACCAA |
| | | TCAGACAAGCATGTACCTGTGCGCCTCTAGCGACTGGCTGGCCGG |
| | | AGCAAAGGATGAGCAGTATTTCGGCCCAGGCACCAGGCTGACCG |
| | | TGACA |
| | 378 | MGIRLLCRVAFCFLAVGLVDVKVTQSSRYLVKRTGEKVFLECVQD |
| | | MDHENMFWYRQDPGLGLRLIYFSYDVKMKEKGDIPEGYSVSREKK |
| | | ERFSLILESASTNQTSMYLCASSDWLAGAKDEQYFGPGTRLTVT |
| RAS TCR-18 | 379 | MTSIRAVFIFLWLQLDLVNGENVEQHPSTLSVQEGDSAVIKCTYSDS |
| alpha chain | | ASNYFPWYKQELGKRPQLIIDIRSNVGEKKDQRIAVTLNKTAKHFSL |
| | | HITETQPEDSAVYFCAETGFQKLVFGTGTRLLVSPDIQNPEPAVYQL |
| | | KDPRSQDSTLCLFTDFDSQINVPKTMESGTFITDKTVLDMKAMDSKS |
| | | NGAIAWSNQTSFTCQDIFKETNATYPSSDVPCDATLTEKSFETDMNL |
| | | NFQNLSVMGLRILLLKVAGFNLLMTLRLWSS |
| RAS TCR-18 | 380 | MGIRLLCRVAFCFLAVGLVDVKVTQSSRYLVKRTGEKVFLECVQD |
| beta chain | | MDHENMFWYRQDPGLGLRLIYFSYDVKMKEKGDIPEGYSVSREKK |
| | | ERFSLILESASTNQTSMYLCASSDWLAGAKDEQYFGPGTRLTVTKDL |
| | | RNVTPPKVSLFEPSKAEIANKQKATLVCLARGFFPDHVELSWWVNG |
| | | KEVHSGVSTDPQAYKESNYSYCLSSRLRVSATFWHNPRNHFRCQVQ |
| | | FHGLSEEDKWPEGSPKPVTQNISAEAWGRADCGITSASYHQGVLSA |
| | | TILYEILLGKATLYAVLVSGLVLMAMVKKKNS |
| TCR | 381 | VVGADGVGK |
| peptides | 382 | VVVGADGVGK |
| RAS TCR-19 | 383 | TSINN |
| alpha chain | | |
| CDR1 | | |
| RAS TCR-19 | 384 | IRSNERE |
| alpha chain | | |
| CDR2 | | |
| RAS TCR-19 | 385 | ATDPLDYKLS |
| alpha chain | | |
| CDR3 | | |
| RAS TCR-19 | 386 | MNHEY |
| beta chain | | |
| CDR1 | | |
| RAS TCR-19 | 387 | SMNVEV |
| beta chain | | |
| CDR2 | | |
| RAS TCR-19 | 388 | ASSLVASNEQF |
| beta chain | | |
| CDR3 | | |
| RAS TCR-19 | 389 | ATGGAAACTCTCCTGGGAGTGTCTTTGGTGATTCTATGGCTTCAA |
| alpha chain | | CTGGCTAGGGTGAACAGTCAACAGGGAGAAGAGGATCCTCAGGC |
| variable | | CTTGAGCATCCAGGAGGGTGAAAATGCCACCATGAACTGCAGTT |
| domains | | ACAAAACTAGTATAAACAATTTACAGTGGTATAGACAAAATTCA |
| | | GGTAGAGGCCTTGTCCACCTAATTTTAATACGTTCAAATGAAAGA |
| | | GAGAAACACAGTGGAAGATTAAGAGTCACGCTTGACACTTCCAA |
| | | GAAAAGCAGTTCCTTGTTGATCACGGCTTCCCGGGCAGCAGACA |
| | | CTGCTTCTTACTTCTGTGCTACGGACCCCTTAGACTACAAGCTCA |
| | | GCTTTGGAGCCGGAACCACAGTAACTGTAAGAGCA |
| | 390 | ATGGAGACCCTGCTGGGCGTGTCTCTGGTCATCCTGTGGCTGCAG |
| | | CTGGCCAGAGTGAACTCTCAGCAGGGAGAGGAGGACCCTCAGGC |
| | | CCTGAGCATCCAGGAGGGCGAGAACGCCACCATGAATTGCTCTT |
| | | ACAAGACAAGCATCAACAATCTGCAGTGGTATCGGCAGAACTCC |
| | | GGCAGAGGCCTGGTGCACCTGATCCTGATCAGGAGCAATGAGCG |
| | | CGAGAAGCACTCCGGCCGGCTGAGAGTGACCCTGGACACATCTA |
| | | AGAAGTCCTCTAGCCTGCTGATCACCGCCTCTAGGGCAGCAGATA |
| | | CAGCCAGCTACTTCTGTGCCACCGACCCACTGGATTATAAGCTGT |
| | | CCTTTGGCGCCGGCACCACAGTGACCGTGCGCGCC |
| | 391 | METLLGVSLVILWLQLARVNSQQGEEDPQALSIQEGENATMNCSY |
| | | KTSINNLQWYRQNSGRGLVHLILIRSNEREKHSGRLRVTLDTSKK |
| | | SSSLLITASRAADTASYFCATDPLDYKLSFGAGTTVTVRA |
| RAS TCR-19 | 392 | ATGGGCCCCCAGCTCCTTGGCTATGTGGTCCTTTGCCTTCTAGGA |
| beta chain | | GCAGGCCCCCTGGAAGCCCAAGTGACCCAGAACCCAAGATACCT |
| variable | | CATCACAGTGACTGGAAAGAAGTTAACAGTGACTTGTTCTCAGA |
| domains | | ATATGAACCATGAGTATATGTCCTGGTATCGACAAGACCCAGGG |
| | | CTGGGCTTAAGGCAGATCTACTATTCAATGAATGTTGAGGTGACT |
| | | GATAAGGGAGATGTTCCTGAAGGGTACAAAGTCTCTCGAAAAGA |
| | | GAAGAGGAATTTCCCCCTGATCCTGGAGTCGCCCAGCCCCAACC |
| | | AGACCTCTCTGTACTTCTGTGCCAGCAGTTTGGTGGCTAGCAATG |
| | | AGCAGTTCTTCGGGCCAGGGACACGGCTCACCGTGCTA |
| | 393 | ATGGGCCCACAGCTGCTGGGCTACGTGGTGCTGTGCCTGCTGGGA |
| | | GCAGGACCACTGGAGGCACAGGTGACCCAGAATCCCCGGTATCT |
| | | GATCACCGTGACAGGCAAGAAGCTGACCGTGACATGTTCCCAGA |
| | | ACATGAATCACGAGTACATGTCTTGGTATAGGCAGGACCCCGGA |
| | | CTGGGACTGAGGCAGATCTACTATTCTATGAACGTGGAGGTGAC |
| | | AGACAAGGGCGATGTGCCTGAGGGCTACAAGGTGAGCAGGAAG |
| | | GAGAAGCGCAACTTCCCACTGATCCTGGAGTCCCCATCTCCCAAT |
| | | CAGACCAGCCTGTATTTTTGCGCCAGCTCCCTGGTGGCCTCCAAC |
| | | GAGCAGTTCTTTGGCCCTGGCACCCGGCTGACAGTGCTG |
| | 394 | MGPQLLGYVVLCLLGAGPLEAQVTQNPRYLITVTGKKLTVTCSQN |
| | | MNHEYMSWYRQDPGLGLRQIYYSMNVEVTDKGDVPEGYKVSRKEK |
| | | RNFPLILESPSPNQTSLYFCASSLVASNEQFFGPGTRLTVL |
| RAS TCR-19 | 395 | METLLGVSLVILWLQLARVNSQQGEEDPQALSIQEGENATMNCSYKT |
| alpha chain | | SINNLQWYRQNSGRGLVHLILIRSNEREKHSGRLRVTLDTSKKSSSL |
| | | LITASRAADTASYFCATDPLDYKLSFGAGTTVTVRADIQNPEPAVYQ |
| | | LKDPRSQDSTLCLFTDFDSQINVPKTMESGTFITDKTVLDMKAMDSK |
| | | SNGAIAWSNQTSFTCQDIFKETNATYPSSDVPCDATLTEKSFETDMN |
| | | LNFQNLSVMGLRILLLKVAGFNLLMTLRLWSS |
| RAS TCR-19 | 396 | MGPQLLGYVVLCLLGAGPLEAQVTQNPRYLITVTGKKLTVTCSQNM |
| beta chain | | NHEYMSWYRQDPGLGLRQIYYSMNVEVTDKGDVPEGYKVSRKEKRN |
| | | FPLILESPSPNQTSLYFCASSLVASNEQFFGPGTRLTVLKDLRNVT |
| | | PPKVSLFEPSKAEIANKQKATLVCLARGFFPDHVELSWWVNGKEVH |
| | | SGVSTDPQAYKESNYSYCLSSRLRVSATFWHNPRNHFRCQVQFHGL |
| | | SEEDKWPEGSPKPVTQNISAEAWGRADCGITSASYHQGVLSATILY |
| | | EILLGKATLYAVLVSGLVLMAMVKKKNS |
| TCR | 397 | VVGAVGVGK |
| Peptides | 398 | VVVGAVGVGK |
| RAS TCR-20 | 399 | TSESDYY |
| alpha chain | | |
| CDR1 | | |
| RAS TCR-20 | 400 | QEAYKQQN |
| alpha chain | | |
| CDR2 | | |
| RAS TCR-20 | 401 | ACQGGSEKLV |
| alpha chain | | |
| CDR3 | | |
| RAS TCR-20 | 402 | SGHNT |
| beta chain | | |
| CDR1 | | |
| RAS TCR-20 | 403 | YYREEE |
| beta chain | | |
| CDR2 | | |
| RAS TCR-20 | 404 | ASSLGLLLYNEQF |
| beta chain | | |
| CDR3 | | |
| RAS TCR-20 | 405 | ATGGCATGCCCTGGCTTCCTGTGGGCACTTGTGATCTCCACCTGT |
| alpha chain | | CTTGAATTTAGCATGGCTCAGACAGTCACTCAGTCTCAACCAGAG |
| variable | | ATGTCTGTGCAGGAGGCAGAGACCGTGACCCTGAGCTGCACATA |
| domains | | TGACACCAGTGAGAGTGATTATTATTTATTCTGGTACAAGCAGCC |
| | | TCCCAGCAGGCAGATGATTCTCGTTATTCGCCAAGAAGCTTATAA |
| | | GCAACAGAATGCAACAGAGAATCGTTTCTCTGTGAACTTCCAGA |
| | | AAGCAGCCAAATCCTTCAGTCTCAAGATCTCAGACTCACAGCTGG |
| | | GGGATGCCGCGATGTATTTCTGTGCTTGTCAGGGCGGATCTGAAA |
| | | AGCTGGTCTTTGGAAAGGGAACGAAACTGACAGTAAACCCA |
| | 406 | ATGGCATGCCCAGGCTTCCTGTGGGCACTGGTCATCAGCACCTGT |
| | | CTGGAGTTTTCTATGGCCCAGACCGTGACACAGAGCCAGCCAGA |
| | | GATGTCCGTGCAGGAGGCAGAGACCGTGACACTGTCCTGTACCT |
| | | ACGACACAAGCGAGTCCGATTACTATCTGTTCTGGTATAAGCAGC |
| | | CTCCATCTCGCCAGATGATCCTGGTCATCCGGCAGGAGGCCTACA |
| | | AGCAGCAGAACGCCACCGAGAATCGGTTCTCTGTGAATTTTCAGA |
| | | AGGCCGCCAAGTCTTTTAGCCTGAAGATCTCCGACTCTCAGCTGG |
| | | GCGATGCCGCCATGTATTTCTGCGCATGTCAGGGAGGCAGCGAG |
| | | AAGCTGGTGTTTGGCAAGGGCACCAAGCTGACAGTGAACCCT |
| | 407 | MACPGFLWALVISTCLEFSMAQTVTQSQPEMSVQEAETVTLSCTYD |
| | | TSESDYYLFWYKQPPSRQMILVIRQEAYKQQNATENRFSVNFQKAA |
| | | KSFSLKISDSQLGDAAMYFCACQGGSEKLVFGKGTKLTVNP |
| RAS TCR-20 | 408 | ATGGGCCCTGGGCTCCTCTGCTGGGTGCTGCTTTGTCTCCTGGGA |
| beta chain | | GCAGGCTCAGTGGAGACTGGAGTCACCCAAAGTCCCACACACCT |
| variable | | GATCAAAACGAGAGGACAGCAAGTGACTCTGAGATGCTCTTCTC |
| domains | | AGTCTGGGCACAACACTGTGTCCTGGTACCAACAGGCCCTGGGTC |
| | | AGGGGCCCCAGTTTATCTTTCAGTATTATAGGGAGGAAGAGAAT |
| | | GGCAGAGGAAACTTCCCTCCTAGATTCTCAGGTCTCCAGTTCCCT |
| | | AATTATAGCTCTGAGCTGAATGTGAACGCCTTGGAGCTGGACGAC |
| | | TCGGCCCTGTATCTCTGTGCCAGCAGCTTGGGACTCCTCCTCTAC |
| | | AATGAGCAGTTCTTCGGGCCAGGGACACGGCTCACCGTGCTA |
| | 409 | ATGGGACCAGGACTGCTGTGCTGGGTGCTGCTGTGCCTGCTGGGA |
| | | GCAGGCAGCGTGGAGACCGGCGTGACACAGTCCCCTACCCACCT |
| | | GATCAAGACAAGAGGCCAGCAGGTGACCCTGAGGTGCAGCTCCC |
| | | AGTCTGGCCACAATACAGTGAGCTGGTACCAGCAGGCCCTGGGA |
| | | CAGGGACCTCAGTTCATCTTTCAGTACTATAGGGAGGAGGAGAA |
| | | CGGCCGCGGCAATTTCCCCCCTCGGTTTAGCGGCCTGCAGTTCCC |
| | | AAACTATTCTAGCGAGCTGAACGTGAATGCCCTGGAGCTGGACG |
| | | ATTCCGCCCTGTACCTGTGCGCCTCCTCTCTGGGCCTGCTGCTGTA |
| | | TAACGAGCAGTTCTTTGGCCCCGGCACCAGACTGACAGTGCTG |
| | 410 | MGPGLLCWVLLCLLGAGSVETGVTQSPTHLIKTRGQQVTLRCSSQS |
| | | GHNTVSWYQQALGQGPQFIFQYYREEENGRGNFPPRFSGLQFPNYS |
| | | SELNVNALELDDSALYLCASSLGLLLYNEQFFGPGTRLTVL |
| RAS TCR-20 | 411 | MACPGFLWALVISTCLEFSMAQTVTQSQPEMSVQEAETVTLSCTYD |
| alpha chain | | TSESDYYLFWYKQPPSRQMILVIRQEAYKQQNATENRFSVNFQKAA |
| | | KSFSLKISDSQLGDAAMYFCACQGGSEKLVFGKGTKLTVNPDIQNPE |
| | | PAVYQLKDPRSQDSTLCLFTDFDSQINVPKTMESGTFITDKTVLDMK |
| | | AMDSKSNGAIAWSNQTSFTCQDIFKETNATYPSSDVPCDATLTEKSF |
| | | ETDMNLNFQNLSVMGLRILLLKVAGENLLMTLRLWSS |
| RAS TCR-20 | 412 | MGPGLLCWVLLCLLGAGSVETGVTQSPTHLIKTRGQQVTLRCSSQS |
| beta chain | | GHNTVSWYQQALGQGPQFIFQYYREEENGRGNFPPRFSGLQFPNYS |
| | | SELNVNALELDDSALYLCASSLGLLLYNEQFFGPGTRLTVLKDLRNV |
| | | TPPKVSLFEPSKAEIANKQKATLVCLARGFFPDHVELSWWVNGKEV |
| | | HSGVSTDPQAYKESNYSYCLSSRLRVSATFWHNPRNHFRCQVQFHG |
| | | LSEEDKWPEGSPKPVTQNISAEAWGRADCGITSASYHQGVLSATILY |
| | | EILLGKATLYAVLVSGLVLMAMVKKKNS |
| TCR | 413 | VVGAVGVGK |
| Peptides | 414 | VVVGAVGVGK |
| RAS TCR-21 | 415 | TSINN |
| alpha chain | | |
| CDR1 | | |
| RAS TCR-21 | 416 | IRSNERE |
| alpha chain | | |
| CDR2 | | |
| RAS TCR-21 | 417 | ATDAQTGANNLF |
| alpha chain | | |
| CDR3 | | |
| RAS TCR-21 | 418 | SGHAT |
| beta chain | | |
| CDR1 | | |
| RAS TCR-21 | 419 | FQNNGV |
| beta chain | | |
| CDR2 | | |
| RAS TCR-21 | 420 | ASSLGDSYEQYF |
| beta chain | | |
| CDR3 | | |
| RAS TCR-21 | 421 | ATGGAAACTCTCCTGGGAGTGTCTTTGGTGATTCTATGGCTTCAA |
| alpha chain | | CTGGCTAGGGTGAACAGTCAACAGGGAGAAGAGGATCCTCAGGC |
| variable | | CTTGAGCATCCAGGAGGGTGAAAATGCCACCATGAACTGCAGTT |
| domains | | ACAAAACTAGTATAAACAATTTACAGTGGTATAGACAAAATTCA |
| | | GGTAGAGGCCTTGTCCACCTAATTTTAATACGTTCAAATGAAAGA |
| | | GAGAAACACAGTGGAAGATTAAGAGTCACGCTTGACACTTCCAA |
| | | GAAAAGCAGTTCCTTGTTGATCACGGCTTCCCGGGCAGCAGACA |
| | | CTGCTTCTTACTTCTGTGCTACGGACGCTCAAACTGGGGCAAACA |
| | | ACCTCTTCTTTGGGACTGGAACGAGACTCACCGTTATTCCC |
| | 422 | ATGGAGACACTGCTGGGCGTGTCTCTGGTCATCCTGTGGCTGCAG |
| | | CTGGCCAGAGTGAATAGCCAGCAGGGAGAGGAGGACCCCCAGG |
| | | CCCTGTCCATCCAGGAGGGCGAGAACGCCACCATGAATTGCAGC |
| | | TACAAGACATCCATCAACAATCTGCAGTGGTATCGGCAGAACTCT |
| | | GGCAGAGGCCTGGTGCACCTGATCCTGATCCGGTCCAATGAGAG |
| | | AGAGAAGCACTCTGGCCGGCTGAGAGTGACCCTGGATACATCCA |
| | | AGAAGTCCTCTAGCCTGCTGATCACCGCCAGCCGGGCAGCAGAC |
| | | ACAGCCTCCTATTTTTGTGCCACCGATGCCCAGACAGGCGCCAAC |
| | | AATCTGTTCTTTGGCACCGGCACAAGACTGACCGTGATCCCT |
| | 423 | METLLGVSLVILWLQLARVNSQQGEEDPQALSIQEGENATMNCS |
| | | YKTSINNLQWYRQNSGRGLVHLILIRSNEREKHSGRLRVTLDTS |
| | | KKSSSLLITASRAADTASYFCATDAQTGANNLFFGTGTRLTVIP |
| RAS TCR-21 | 424 | ATGGGCACCAGGCTCCTCTGCTGGGCGGCCCTCTGTCTCCTGGGA |
| beta chain | | GCAGAACTCACAGAAGCTGGAGTTGCCCAGTCTCCCAGATATAA |
| variable | | GATTATAGAGAAAAGGCAGAGTGTGGCTTTTTGGTGCAATCCTAT |
| domains | | ATCTGGCCATGCTACCCTTTACTGGTACCAGCAGATCCTGGGACA |
| | | GGGCCCAAAGCTTCTGATTCAGTTTCAGAATAACGGTGTAGTGGA |
| | | TGATTCACAGTTGCCTAAGGATCGATTTTCTGCAGAGAGGCTCAA |
| | | AGGAGTAGACTCCACTCTCAAGATCCAGCCTGCAAAGCTTGAGG |
| | | ACTCGGCCGTGTATCTCTGTGCCAGCAGCTTA |
| | 425 | ATGGGCACCAGGCTGCTGTGCTGGGCCGCCCTGTGCCTGCTGGGA |
| | | GCAGAGCTGACAGAGGCAGGAGTGGCCCAGAGCCCCAGGTACA |
| | | AGATCATCGAGAAGCGCCAGTCCGTGGCCTTCTGGTGCAACCCTA |
| | | TCTCTGGCCACGCCACCCTGTACTGGTATCAGCAGATCCTGGGCC |
| | | AGGGCCCAAAGCTGCTGATCCAGTTCCAGAACAATGGCGTGGTG |
| | | GACGATTCTCAGCTGCCCAAGGACAGGTTTAGCGCCGAGCGCCT |
| | | GAAGGGCGTGGATTCTACCCTGAAGATCCAGCCAGCAAAGCTGG |
| | | AGGACAGCGCCGTGTACCTGTGCGCCAGCTCCCTG |
| | 426 | MGTRLLCWAALCLLGAELTEAGVAQSPRYKIIEKRQSVAFWCNPIS |
| | | GHATLYWYQQILGQGPKLLIQFQNNGVVDDSQLPKDRFSAERLKGV |
| | | DSTLKIQPAKLEDSAVYLCASSL |
| RAS TCR-21 | 427 | METLLGVSLVILWLQLARVNSQQGEEDPQALSIQEGENATMNCSYK |
| alpha chain | | TSINNLQWYRQNSGRGLVHLILIRSNEREKHSGRLRVTLDTSKKSSSL |
| | | LITASRAADTASYFCATDAQTGANNLFFGTGTRLTVIPDIQNPEPAV |
| | | YQLKDPRSQDSTLCLFTDFDSQINVPKTMESGTFITDKTVLDMKAM |
| | | DSKSNGAIAWSNQTSFTCQDIFKETNATYPSSDVPCDATLTEKSFET |
| | | DMNLNFQNLSVMGLRILLLKVAGFNLLMTLRLWSS |
| RAS TCR-21 | 428 | MGTRLLCWAALCLLGAELTEAGVAQSPRYKIIEKRQSVAFWCNPIS |
| beta chain | | GHATLYWYQQILGQGPKLLIQFQNNGVVDDSQLPKDRFSAERLKGV |
| | | DSTLKIQPAKLEDSAVYLCASSLKDLRNVTPPKVSLFEPSKAEIANKQ |
| | | KATLVCLARGFFPDHVELSWWVNGKEVHSGVSTDPQAYKESNYSY |
| | | CLSSRLRVSATFWHNPRNHFRCQVQFHGLSEEDKWPEGSPKPVTQN |
| | | ISAEAWGRADCGITSASYHQGVLSATILYEILLGKATLYAVLVSGLV |
| | | LMAMVKKKNS |
| TCR | 429 | VVGAVGVGK |
| Peptides | 430 | VVVGAVGVGK |
| RAS TCR-26 | 532 | NSMFDY |
| alpha chain | | |
| CDR1 | | |
| RAS TCR-26 | 533 | ISSIKDK |
| alpha chain | | |
| CDR2 | | |
| RAS TCR-26 | 534 | AANAGGTSYGKLT |
| alpha chain | | |
| CDR3 | | |
| RAS TCR-26 | 535 | SNHLY |
| beta chain | | |
| CDR1 | | |
| RAS TCR-26 | 536 | FYNNEI |
| beta chain | | |
| CDR2 | | |
| RAS TCR-26 | 537 | ASSEWGSTGELF |
| beta chain | | |
| CDR3 | | |
| RAS TCR-26 | 538 | ATGGCCATGCTCCTGGGGGCATCAGTGCTGATTCTGTGGCTTCAG |
| alpha chain | | CCAGACTGGGTAAACAGTCAACAGAAGAATGATGACCAGCAAGT |
| variable | | TAAGCAAAATTCACCATCCCTGAGCGTCCAGGAAGGAAGAATTT |
| domain | | CTATTCTGAACTGTGACTATACTAACAGCATGTTTGATTATTTCCT |
| | | ATGGTACAAAAAATACCCTGCTGAAGGTCCTACATTCCTGATATC |
| | | TATAAGTTCCATTAAGGATAAAAATGAAGATGGAAGATTCACTG |
| | | TCTTCTTAAACAAAAGTGCCAAGCACCTCTCTCTGCACATTGTGC |
| | | CCTCCCAGCCTGGAGACTCTGCAGTGTACTTCTGTGCAGCAAATG |
| | | CTGGTGGTACTAGCTATGGAAAGCTGACATTTGGACAAGGGACC |
| | | ATCTTGACTGTCCATCCAA |
| | 539 | MAMLLGASVLILWLQPDWVNSQQKNDDQQVKQNSPSLSVQEGRISI |
| | | LNCDYTNSMFDYFLWYKKYPAEGPTFLISISSIKDKNEDGRFTVFLN |
| | | KSAKHLSLHIVPSQPGDSAVYFCAANAGGTSYGKLTFGQGTILTVHP |
| RAS TCR-26 | 540 | ATGGATACCTGGCTCGTATGCTGGGCAATTTTTAGTCTCTTGAAA |
| beta chain | | GCAGGACTCACAGAACCTGAAGTCACCCAGACTCCCAGCCATCA |
| variable | | GGTCACACAGATGGGACAGGAAGTGATCTTGCGCTGTGTCCCCAT |
| domain | | CTCTAATCACTTATACTTCTATTGGTACAGACAAATCTTGGGGCA |
| | | GAAAGTCGAGTTTCTGGTTTCCTTTTATAATAATGAAATCTCAGA |
| | | GAAGTCTGAAATATTCGATGATCAATTCTCAGTTGAAAGGCCTGA |
| | | TGGATCAAATTTCACTCTGAAGATCCGGTCCACAAAGCTGGAGG |
| | | ACTCAGCCATGTACTTCTGTGCCAGCAGTGAATGGGGAAGCACC |
| | | GGGGAGCTGTTTTTTGGAGAAGGCTCTAGGCTGACCGTACTGG |
| | 541 | MDTWLVCWAIFSLLKAGLTEPEVTQTPSHQVTQMGQEVILRCVPISN |
| | | HLYFYWYRQILGQKVEFLVSFYNNEISEKSEIFDDQFSVERPDGSNFT |
| | | LKIRSTKLEDSAMYFCASSEWGSTGELFFGEGSRLTVL |
| RAS TCR-26 | 542 | MAMLLGASVLILWLQPDWVNSQQKNDDQQVKQNSPSLSVQEGRISI |
| alpha chain | | LNCDYTNSMFDYFLWYKKYPAEGPTFLISISSIKDKNEDGRFTVFLN |
| | | KSAKHLSLHIVPSQPGDSAVYFCAANAGGTSYGKLTFGQGTILTVHP |
| | | DIQNPEPAVYQLKDPRSQDSTLCLFTDFDSQINVPKTMESGTFITDKT |
| | | VLDMKAMDSKSNGAIAWSNQTSFTCQDIFKETNATYPSSDVPCDAT |
| | | LTEKSFETDMNLNFQNLSVMGLRILLLKVAGFNLLMTLRLWSS |
| RAS TCR-26 | 543 | MDTWLVCWAIFSLLKAGLTEPEVTQTPSHQVTQMGQEVILRCVPISN |
| beta chain | | HLYFYWYRQILGQKVEFLVSFYNNEISEKSEIFDDQFSVERPDGSNFT |
| | | LKIRSTKLEDSAMYFCASSEWGSTGELFFGEGSRLTVLKDLRNVTPP |
| | | KVSLFEPSKAEIANKQKATLVCLARGFFPDHVELSWWVNGKEVHSG |
| | | VSTDPQAYKESNYSYCLSSRLRVSATFWHNPRNHFRCQVQFHGLSE |
| | | EDKWPEGSPKPVTQNISAEAWGRADCGITSASYHQGVLSATILYEIL |
| | | LGKATLYAVLVSGLVLMAMVKKKNS |
| RAS TCR-26 | 544 | VVGAVGVGK |
| peptide | | |
| RAS TCR-27 | 545 | TTSDR |
| alpha chain | | |
| CDR1 | | |
| RAS TCR-27 | 546 | LLSNGAV |
| alpha chain | | |
| CDR2 | | |
| RAS TCR-27 | 547 | AVDIIGGKST |
| alpha chain | | |
| CDR3 | | |
| RAS TCR-27 | 548 | SNHLY |
| beta chain | | |
| CDR1 | | |
| RAS TCR-27 | 549 | FYNNEI |
| beta chain | | |
| CDR2 | | |
| RAS TCR-27 | 550 | ASSEWGSTGELF |
| beta chain | | |
| CDR3 | | |
| RAS TCR-27 | 551 | ATGAAGAAGCTACTAGCAATGATTCTGTGGCTTCAACTAGACCGG |
| alpha chain | | TTAAGTGGAGAGCTGAAAGTGGAACAAAACCCTCTGTTCCTGAG |
| variable | | CATGCAGGAGGGAAAAAACTATACCATCTACTGCAATTATTCAA |
| domain | | CCACTTCAGACAGACTGTATTGGTACAGGCAGGATCCTGGGAAA |
| | | AGTCTGGAATCTCTGTTTGTGTTGCTATCAAATGGAGCAGTGAAG |
| | | CAGGAGGGACGATTAATGGCCTCACTTGATACCAAAGCCCGTCT |
| | | CAGCACCCTCCACATCACAGCTGCCGTGCATGACCTCTCTGCCAC |
| | | CTACTTCTGTGCCGTGGACATCATCGGAGGCAAATCAACCTTTGG |
| | | GGATGGGACTACGCTCACTGTGAAGCCAA |
| | 552 | MKKLLAMILWLQLDRLSGELKVEQNPLFLSMQEGKNYTIYCNYSTT |
| | | SDRLYWYRQDPGKSLESLFVLLSNGAVKQEGRLMASLDTKARLSTL |
| | | HITAAVHDLSATYFCAVDIIGGKSTFGDGTTLTVKP |
| RAS TCR-27 | 553 | ATGGATACCTGGCTCGTATGCTGGGCAATTTTTAGTCTCTTGAAA |
| beta chain | | GCAGGACTCACAGAACCTGAAGTCACCCAGACTCCCAGCCATCA |
| variable | | GGTCACACAGATGGGACAGGAAGTGATCTTGCGCTGTGTCCCCAT |
| domain | | CTCTAATCACTTATACTTCTATTGGTACAGACAAATCTTGGGGCA |
| | | GAAAGTCGAGTTTCTGGTTTCCTTTTATAATAATGAAATCTCAGA |
| | | GAAGTCTGAAATATTCGATGATCAATTCTCAGTTGAAAGGCCTGA |
| | | TGGATCAAATTTCACTCTGAAGATCCGGTCCACAAAGCTGGAGG |
| | | ACTCAGCCATGTACTTCTGTGCCAGCAGTGAATGGGGAAGCACC |
| | | GGGGAGCTGTTTTTTGGAGAAGGCTCTAGGCTGACCGTACTGG |
| | 554 | MDTWLVCWAIFSLLKAGLTEPEVTQTPSHQVTQMGQEVILRCV |
| | | PISNHLYFYWYRQILGQKVEFLVSFYNNEISEKSEIFDDQFSV |
| | | ERPDGSNFTLKIRSTKLEDSAMYFCASSEWGSTGELFFGEGSR |
| | | LTVL |
| RAS TCR-27 | 555 | MKKLLAMILWLQLDRLSGELKVEQNPLFLSMQEGKNYTIYCNYSTT |
| alpha chain | | SDRLYWYRQDPGKSLESLFVLLSNGAVKQEGRLMASLDTKARLSTL |
| | | HITAAVHDLSATYFCAVDIIGGKSTFGDGTTLTVKPDIQNPEPAVYQL |
| | | KDPRSQDSTLCLFTDFDSQINVPKTMESGTFITDKTVLDMKAMDSKS |
| | | NGAIAWSNQTSFTCQDIFKETNATYPSSDVPCDATLTEKSFETDMNL |
| | | NFQNLSVMGLRILLLKVAGFNLLMTLRLWSS |
| RAS TCR-27 | 556 | MDTWLVCWAIFSLLKAGLTEPEVTQTPSHQVTQMGQEVILRCVPISN |
| beta chain | | HLYFYWYRQILGQKVEFLVSFYNNEISEKSEIFDDQFSVERPDGSNFT |
| | | LKIRSTKLEDSAMYFCASSEWGSTGELFFGEGSRLTVLKDLRNVTPP |
| | | KVSLFEPSKAEIANKQKATLVCLARGFFPDHVELSWWVNGKEVHSG |
| | | VSTDPQAYKESNYSYCLSSRLRVSATFWHNPRNHFRCQVQFHGLSE |
| | | EDKWPEGSPKPVTQNISAEAWGRADCGITSASYHQGVLSATILYEIL |
| | | LGKATLYAVLVSGLVLMAMVKKKNS |
| RAS TCR-27 | 557 | VVGAVGVGK |
| peptide | | |
| RAS TCR-28 | 558 | NSMFDY |
| alpha chain | | |
| CDR1 | | |
| RAS TCR-28 | 559 | ISSIKDK |
| alpha chain | | |
| CDR2 | | |
| RAS TCR-28 | 560 | AASAVGQEYGNKLV |
| alpha chain | | |
| CDR3 | | |
| RAS TCR-28 | 561 | SEHNR |
| beta chain | | |
| CDR1 | | |
| RAS TCR-28 | 562 | FQNEAQ |
| beta chain | | |
| CDR2 | | |
| RAS TCR-28 | 563 | ASSEYTMGTQY |
| beta chain | | |
| CDR3 | | |
| RAS TCR-28 | 564 | ATGGCCATGCTCCTGGGGGCATCAGTGCTGATTCTGTGGCTTCAG |
| alpha chain | | CCAGACTGGGTAAACAGTCAACAGAAGAATGATGACCAGCAAGT |
| variable | | TAAGCAAAATTCACCATCCCTGAGCGTCCAGGAAGGAAGAATTT |
| domain | | CTATTCTGAACTGTGACTATACTAACAGCATGTTTGATTATTTCCT |
| | | ATGGTACAAAAAATACCCTGCTGAAGGTCCTACATTCCTGATATC |
| | | TATAAGTTCCATTAAGGATAAAAATGAAGATGGAAGATTCACTG |
| | | TCTTCTTAAACAAAAGTGCCAAGCACCTCTCTCTGCACATTGTGC |
| | | CCTCCCAGCCTGGAGACTCTGCAGTGTACTTCTGTGCAGCAAGCG |
| | | CAGTAGGTCAGGAATATGGAAACAAGCTGGTCTTTGGCGCAGGA |
| | | ACCATTCTGAGAGTCAAGTCCT |
| | 565 | MAMLLGASVLILWLQPDWVNSQQKNDDQQVKQNSPSLSVQEGRI |
| | | SILNCDYTNSMFDYFLWYKKYPAEGPTFLISISSIKDKNEDGRF |
| | | TVFLNKSAKHLSLHIVPSQPGDSAVYFCAASAVGQEYGNKLVFG |
| | | AGTILRVKS |
| RAS TCR-28 | 566 | ATGGGCACCAGCCTCCTCTGCTGGATGGCCCTGTGTCTCCTGGGG |
| beta chain | | GCAGATCACGCAGATACTGGAGTCTCCCAGAACCCCAGACACAA |
| variable | | GATCACAAAGAGGGGACAGAATGTAACTTTCAGGTGTGATCCAA |
| domain | | TTTCTGAACACAACCGCCTTTATTGGTACCGACAGACCCTGGGGC |
| | | AGGGCCCAGAGTTTCTGACTTACTTCCAGAATGAAGCTCAACTAG |
| | | AAAAATCAAGGCTGCTCAGTGATCGGTTCTCTGCAGAGAGGCCT |
| | | AAGGGATCTTTCTCCACCTTGGAGATCCAGCGCACAGAGCAGGG |
| | | GGACTCGGCCATGTATCTCTGTGCCAGCAGTGAATATACTATGGG |
| | | GACCCAGTACTTCGGGCCAGGCACGCGGCTCCTGGTGCTCG |
| RAS TCR-28 | 567 | MGTSLLCWMALCLLGADHADTGVSQNPRHKITKRGQNVTFRCDPIS |
| beta chain | | EHNRLYWYRQTLGQGPEFLTYFQNEAQLEKSRLLSDRFSAERPKGS |
| variable | | FSTLEIQRTEQGDSAMYLCASSEYTMGTQYFGPGTRLLVL |
| domain | | |
| RAS TCR-28 | 568 | MAMLLGASVLILWLQPDWVNSQQKNDDQQVKQNSPSLSVQEGRISI |
| alpha chain | | LNCDYTNSMFDYFLWYKKYPAEGPTFLISISSIKDKNEDGRFTVFL |
| | | NKSAKHLSLHIVPSQPGDSAVYFCAASAVGQEYGNKLVFGAGTILR |
| | | VKSDIQNPEPAVYQLKDPRSQDSTLCLFTDFDSQINVPKTMESGTF |
| | | ITDKTVLDMKAMDSKSNGAIAWSNQTSFTCQDIFKETNATYPSSDV |
| | | PCDATLTEKSFETDMNLNFQNLSVMGLRILLLKVAGFNLLMTLRLW |
| | | SS |
| RAS TCR-28 | 569 | MGTSLLCWMALCLLGADHADTGVSQNPRHKITKRGQNVTFRCDPIS |
| beta chain | | EHNRLYWYRQTLGQGPEFLTYFQNEAQLEKSRLLSDRFSAERPKGS |
| | | FSTLEIQRTEQGDSAMYLCASSEYTMGTQYFGPGTRLLVLKDLRNV |
| | | TPPKVSLFEPSKAEIANKQKATLVCLARGFFPDHVELSWWVNGKEV |
| | | HSGVSTDPQAYKESNYSYCLSSRLRVSATFWHNPRNHFRCQVQFHG |
| | | LSEEDKWPEGSPKPVTQNISAEAWGRADCGITSASYHQGVLSATILY |
| | | EILLGKATLYAVLVSGLVLMAMVKKKNS |
| RAS TCR-28 | 600 | VVGAVGVGK |
| peptide | | |

In some embodiments, the antigen is an ovarian cancer antigen or a T lymphoma antigen.

In some embodiments, the TRAV:TRBV comprises sequences for binding a G12D mutated RAS antigen (GADGVGKSA) presented on an MHC protein encoded by an HLA:C*08:02. The

TRAV comprises a sequence:
LAKTTQPISVDSYEGQEVNITCSHNNIATNDYITWYQQFPSQGP
RFIIQGYKTKVTNEVASLFIPADRKSSTLSLPRVSLSDTAVYYC
LVGDMDQAGTALIFGKGTTLSVSSD (SEQ ID NO: 619).
The respective CDR sequences are:
CDR1:
NIATNDY (SEQ ID NO: 874)
CDR2:
GYKTK (SEQ ID NO: 875)
CDR3:
LVGDMDQAGTALI (SEQ ID NO: 876):
(CDR sequences according to IMGT)
The TRBV comprises a sequence:
AGVTQSPTHLIKTRGQQVTLRCSPKSGHDTVSWYQQALGQGPQF
IFQYYEEEERQRGNFPDRFSGHQFPNYSSELNVNALLLGDSALY
LCASSLGEGRVDGYTFGSGTRLTVV
(SEQ ID NO: 620).
The respective CDR sequences are:
CDR1:
SGHDT (SEQ ID NO: 877)
CDR2:
YYEEEE (SEQ ID NO: 878)
CDR3:
ASSLGEGRVDGYT (SEQ ID NO: 879);
(CDR sequences according to IMGT)

In some embodiments, the TRAV:TRBV may comprise a sequence for binding specificity for any other non-RAS epitope, as might be necessary. For example, a TRAV:TRBV for the multispecific recombinant soluble polypeptide may comprise a binding specificity for MART1 epitope, presented on an MHC protein encoded by an HLA:A*02:01. The antigen binding affinity (KD) of pHLA in this case is 16 nM. For example, a TCR may comprise a TRAV domain having a sequence of: QQKEVEQNSGPLSVPEGAIASLNCTYSDRGSQSFFWYRQYSGKSPELIMSIYSNGDKEDGRF TAQLNKASQYVSLLIRDSQPSDSATYLCAGGGGADGLTFGKGTHLIIQP (SEQ ID NO: 621), or a sequence that is at least 80% identical to SEQ ID NO: 621; and a TRBV domain, having a sequence of: GITQSPKYLFRKEGQNVTLSCEQNLNHDAMYWYRQDPGQGLRLIYYSQIVNDFQKGDIAEG YSVSREKKESFPLTVTSAQKNPTAFYLCASSQGLAGAGELFFGEGSRLTVL (SEQ ID NO: 622), or a sequence that is at least 80% identical to SEQ ID NO: 622, wherein the TRAV:TRBV binds to a MART1 epitope.

In some embodiments, the TRAV:TRBV comprises sequences for binding a Claudin epitope. In one embodiment said claudin is expressed in a cancer cell. In one embodiment said claudin is expressed on the surface of a cancer cell. In one embodiment said claudin is selected from the group consisting of claudin 18.2 and claudin 6. In one embodiment said first binding domain binds to an extracellular domain of said claudin. In one embodiment said first binding domain binds to native epitopes of surface of living cells. In one embodiment the binding agent comprises a variable domain of a heavy chain of an immunoglobulin (VH) with a specificity for a claudin antigen (VH(CLDN)), a variable domain of a light chain of an immunoglobulin (VL) with a specificity for a claudin antigen (VL(CLDN)).

### II. T Cell Engager

In some embodiments, the bispecific T cell engager is designed to strengthen a TCR-MHC synapse of a T cell: cancer cell interaction and facilitates activation of the T cell sufficiently to promote activation and killing of the target cancer cell. The basic observations that necessitates the work disclosed herein is drawn from the general observation that a TCR-MHC peptide interaction is a low affinity interaction. TCR binding facilitated by CD4/CD8 coreceptor by to MHC (avidity, off-rate). Synapse formation and killing requires one or more integration of signals over time (repeated engagement). Besides, low density of MHC:peptides are sufficient to enable recognition/killing Adhesion molecules interactions prolong and facilitate T cell target interaction. The bi- or trispecific engagers prolong and facilitate T cell target interaction through additional anchoring. In general, the co-stimutory receptor interactions fine-tune quality of the T cell response. One function sought to be achieved herein is the sufficient activation of the T cell to generate an effective response that destroys the target cell. Hence an engager of the bi- or trispecific engager is a T cell activator, e.g., a ligand.

In some embodiments, the bispecific engager comprises one or more binding domains, wherein the binding domains comprise an scFv, an sdAb, a VHH or a combination thereof.

In contrast, in the scFv construct, but V_{L} and V_{H} domains of antibodies are included in a single polypeptide chain. The two domains are separated by flexible linkers long enough to allow self-assembly of the V_{L} and V_{H} domains into functional epitope binding site.

In some embodiments the one or more binding domains of the T cell engager bind to an extracellular domain of an endogenous receptor expressed by a T cell. In some embodiments, the one or more binding domains of the T cell engager bind to an extracellular domain of a receptor selected from a group consisting of CD3, CD2, CD7, CD5, CD4, CD28, ICAM-1 and CD8. In some embodiments, the one or more binding domains of the T cell engager bind to CD3 delta, CD3 gamma or CD3 epsilon. In some embodiments, the first binding domain of the T cell engager that binds to an extracellular domain of a first receptor expressed by a T cell comprises a first VHH, and the second binding domain of the T cell engager that binds to an extracellular domain of a second receptor expressed by a T cell comprises a second VHH. In some embodiments, (i) the C-terminus of the first binding domain of the T cell engager is connected to the N-terminus of the second binding domain of the T cell engager; or (ii) the C-terminus of the first binding domain of the T cell engager is connected to the C-terminus of the second binding domain of the T cell engager. In some embodiments, (i) the N-terminus of the first binding domain of the T cell engager is connected to the N-terminus of the second binding domain of the T cell engager; or (ii) the N-terminus of the first binding domain of the T cell engager is connected to the C-terminus of the second binding domain of the T cell engager. In some embodiments, the first binding domain of the T cell engager that binds to an extracellular domain of a first receptor expressed by a T cell comprises a scFv, and the second binding domain of the T cell engager that binds to an extracellular domain of a second receptor expressed by a T cell comprises a VHH. In some embodiments, the first binding domain that binds to an extracellular domain of the first receptor expressed by a T cell and the second binding domain that binds to an extracellular domain of the second receptor expressed by a T cell are connected by a peptide linker.

In some embodiments, the first binding domain on a T cell engager may be connected to a second binding domain via a flexible peptide linker. In some embodiments, the flexible linker may be between 2-50 amino acids long.

An scFv comprises a light chain variable domain, and a heavy chain variable domain. In some embodiments, the light chain variable domain, and the heavy chain variable domain may comprise a short peptide linker comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acids between the light chain variable domain, and the heavy chain variable domain.

In certain embodiments, the scFvs are humanized.

Humanized scFvs comprise “complementarity determining regions” (CDR) that are present on a framework of an immunoglobulin of a different species as compared to that of the parent immunoglobulin from which the CDR was derived. For example, a murine CDR may be grafted into the framework region of a human antibody to prepare the “humanized antibody.”

In some embodiments, the short linker operably linking (a) and (b) may further have additional functions. In some embodiments, the peptides can bind to a specific cell surface receptor, such as, for example, a CD3 receptor, and can activate a receptor mediated cell signaling pathway in the T cell. In some embodiments, the linker is designed such as to be able to bind and activate at least an inflammatory pathway in the T cell or potentiate T cell mediated killing of a target cell. In some embodiments, the linker peptide may have a function of blocking or inhibiting a target cell mediated downregulation of a T cell function. In some embodiments, the nucleic acid constructs for expressing a bispecific scFv engager comprises an N terminal signal peptide sequence for secretion of the bispecific scFv engager.

In one embodiment, an exemplary CD3 binding domain is an scFv. In one embodiment, the anti-CD3 binder is an scFV, the particular domains and sub-domains, respective sequences and/or variations are listed in Table 6A. In one embodiment the anti-CD3 scFv comprises a sequence that is at least 80% identical to SEQ ID NO: 601. In some embodiments, an exemplary CD3 binding domain is an scFv comprising a sequence that is at least 85%, at least 90%, at least 91%, 92%, 93%, 94%, 95%, 96%, 97%, 98%, 99% identical to SEQ ID NO: 601. In one embodiment the anti-CD3 scFv comprises a sequence that is at least 80% identical to SEQ ID NO: 602. In some embodiments, an exemplary CD3 binding domain is an scFv comprising a sequence that is at least 85%, at least 90%, at least 91%, 92%, 93%, 94%, 95%, 96%, 97%, 98%, 99% identical to SEQ ID NO: 602. In one embodiment the anti-CD3 scFv comprises a sequence that is at least 80% identical to SEQ ID NO: 612, 613 or 614. In some embodiments, an exemplary CD3 binding domain is an scFv comprising a sequence that is at least 85%, at least 90%, at least 91%, 92%, 93%, 94%, 95%, 96%, 97%, 98%, 99% identical to SEQ ID NO: 612. In some embodiments, an exemplary CD3 binding domain is an scFv comprising a sequence that is at least 85%, at least 90%, at least 91%, 92%, 93%, 94%, 95%, 96%, 97%, 98%, 99% identical to SEQ ID NO: 613. In some embodiments, an exemplary CD3 binding domain is an scFv comprising a sequence that is at least 85%, at least 90%, at least 91%, 92%, 93%, 94%, 95%, 96%, 97%, 98%, 99% identical to SEQ ID NO: 614. In some embodiments, an exemplary CD3 binding domain is an scFv comprising a sequence of SEQ ID NO: 601, 602, 612, 613, or 614. In some embodiments, an exemplary CD3 binding domain comprises a heavy variable domain that is at least 80% identical to SEQ ID NO: 603. In some embodiments, an exemplary CD3 binding domain comprises a heavy variable domain that is at least 85% identical to SEQ ID NO: 603. In some embodiments, an exemplary CD3 binding domain comprises a heavy variable domain that is at least 90% identical to SEQ ID NO: 603. In some embodiments, an exemplary CD3 binding domain comprises a heavy variable domain that is at least 91%, 92%, 93%, 94%, 95%, 96%, 97%, 98%, 99% identical to SEQ ID NO: 603. In some embodiments, an exemplary CD3 binding domain comprises a heavy variable domain having a sequence of SEQ ID NO: 603. In some embodiments, an exemplary CD3 binding domain comprises a light variable domain that is at least 80% identical to SEQ ID NO: 604. In some embodiments, an exemplary CD3 binding domain comprises a light variable domain that is at least 90% identical to SEQ ID NO: 604. In some embodiments, an exemplary CD3 binding domain comprises alight variable domain that is at least 91%, 92%, 93%, 94%, 95%, 96%, 97%, 98%, 99% identical to SEQ ID NO: 604.

TABLE 6A
CD3 binding domains and sequences
Anti-CD3 binding domains -scFv (IMC)

| | |
|---|---|
| Anti-CD3-scFv-1 | AIQMTQSPSSLSASVGDRVTITCRASQDIRNYLNWYQQKPGKAP |
| VL-VH | KLLIYYTSRLESGVPSRFSGSGSGTDYTLTISSLQPEDFATYYCQQ |
| | GNTLPWTFGQGTKVEIKGGGGSGGGGSGGGGSGGGGSGGGSEV |
| | QLVESGGGLVQPGGSLRLSCAASGYSFTGYTMNWVRQAPGKGL |
| | EWVALINPYKGVSTYNQKFKDRFTISVDKSKNTAYLQMNSLRAE |
| | DTAVYYCARSGYYGDSDWYFDVWGQGTLVTVSS |
| | (SEQ ID NO: 601) |
| Anti CD3-scFv-2 | EVQLVESGGGLVQPGGSLRLSCAASGYSFTGYTMNWVRQAPGK |
| VH-VL | GLEWVALINPYKGVSTYNQKFKDRFTISVDKSKNTAYLQMNSLR |
| | AEDTAVYYCARSGYYGDSDWYFDVWGQGTLVTVSSGGGGSGG |
| | GGSGGGGSGGGGSGGGSAIQMTQSPSSLSASVGDRVTITCRASQ |
| | DIRNYLNWYQQKPGKAPKLLIYYTSRLESGVPSRFSGSGSGTDYT |
| | LTISSLQPEDFATYYCQQGNTLPWTFGQGTKVEIK |
| | (SEQ ID NO: 602) |
| Anti CD3-scFv | EVQLVESGGGLVQPGGSLRLSCAASGYSFTGYTMNWVRQAPGK |
| VH | GLEWVALINPYKGVSTYNQKFKDRFTISVDKSKNTAYLQMNSLR |
| | AEDTAVYYCARSGYYGDSDWYFDVWGQGTLVTVSS |
| | (SEQ ID NO: 603) |
| Anti CD3-scFv | AIQMTQSPSSLSASVGDRVTITCRASQDIRNYLNWYQQKPGKAP |
| VL | KLLIYYTSRLESGVPSRFSGSGSGTDYTLTISSLQPEDFATYYCQQ |
| | GNTLPWTFGQGTKVEIK (SEQ ID NO: 604) |
| HCDR1 | GYTMN (SEQ ID NO: 605)(Kabat)/ |
| | GYSFTGYT (SEQ ID NO: 729)(IMGT) |
| HCDR2 | LINPYKGVSTYNQKFKD (SEQ ID NO: 606)(Kabat)/ |
| | INPYKGVS (SEQ ID NO: 730)(IMGT) |
| HCDR3 | SGYYGDSDWYFDV (SEQ ID NO: 607)(Kabat)/ |
| | ARSGYYGDSDWYFDV (SEQ ID NO: 731)(IMGT) |
| LCDR1 | RASQDIRNYLN (SEQ ID NO: 608)(Kabat)/ |
| | QDIRNY (SEQ ID NO: 732)(IMGT) |
| LCDR2 | YTSRLES (SEQ ID NO: 609)/ |
| | YTS (SEQ ID NO: 733)(IMGT) |
| LCDR3 | QQGNTLPWT (SEQ ID NO: 610)(Kabat/IMGT) |
| Linker joining VH-VL | GGGGSGGGGSGGGGSGGGGSGGGGS (SEQ ID NO: 611) |
| Anti-CD3-scFv-3 | DQQMTQSPSSLSASVGDRVTITCRASQDIRNYLNWYQQKPGKAP |
| LCDRs 1, 2, 3 and | KLLIYYTSRLESGVPSRFSGSGSGTDYTLTISSLQPEDFATYYCQQ |
| HCDRs 1, 2, 3 are | GNTLPWTFGQGTKVEIKGGGGSGGGGSGGGGSGGGGSGGGSEV |
| underlined following | QLVESGGGLVQPGGSLRLSCAASGYSFTGYTMNWVRQAPGKGL |
| IMGT scheme | EWVALINPYKGVSTYNQKFKDRFTISVDKSKNTAYLQMNSLRAE |
| | DTAVYYCARSGYYGDSDWYFDVWGQGTLVTVSS |
| | (SEQ ID NO: 612) |
| Anti-CD3-scFv-4 | DIQMTQSPSSLSASVGDRVTITCRASQDIRNYLNWYQQKPGKAP |
| LCDRs 1, 2, 3 and | KLLIYYTSRLESGVPSRFSGSGSGTDYTLTISSLQPEDFATYYCQQ |
| HCDRs 1, 2, 3 are | GNTLPWTFGQGTKVEIKGGGGSGGGGSGGGGSGGGGSGGGSEV |
| underlined following | QLVESGGGLVQPGGSLRLSCAASGYSFTGYTMNWVRQAPGKGL |
| IMGT scheme | EWVALINPYKGVSTYNQKFKDRFTISVDKSKNTAYLQMNSLRAE |
| | DTAVYYCARSGYYGDSDWYFDVWGQGTLVTVSS |
| | (SEQ ID NO: 613) |
| Anti-CD3-scFv-5 | AQDIQMTQSPSSLSASVGDRVTITCRASQDIRNYLNWYQQKPGK |
| LCDRs 1, 2, 3 and | APKLLIYYTSRLESGVPSRFSGSGSGTDYTLTISSLQPEDFATYYC |
| HCDRs 1, 2, 3 are | QQGNTLPWTFGQGTKVEIKGGGGSGGGGSGGGGSGGGGSGGGS |
| underlined following | EVOLVESGGGLVQPGGSLRLSCAASGYSFTGYTMNWVRQAPGK |
| IMGT scheme | GLEWVALINPYKGVSTYNQKFKDRFTISVDKSKNTAYLQMNSLR |
| | AEDTAVYYCARSGYYGDSDWYFDVWGQGTLVTVSS |
| | (SEQ ID NO: 614) |

In one embodiment, an exemplary CD3 binding domain is an VHH. In one embodiment, the anti-CD3 binder is an VHH, the particular domains and sub-domains, respective sequences and/or variations are listed in Table 6B.

In some embodiments, an exemplary CD3 binding domain is a VHH. In some embodiments, the CD3 binding VHH comprises a heavy chain variable domain that is at least 8000 identical to SEQ ID NO: 615. In some embodiments, an exemplary CD3 binding domain comprises a heavy variable domain that is at least 90% identical to SEQ ID NO: 615. In some embodiments, an exemplary CD3 binding domain comprises a heavy variable domain that is at least 9100, 9200, 9300, 94%, 95%, 96%, 97%, 98%, 99% identical to SEQ ID NO: 615.

In some embodiments, an exemplary CD3 binder is a VHH, and the CD3 binding domain comprises a HCDR3 of SEQ ID NO: 618. In some embodiments, the exemplary CD3 VHH comprises an HCDR1 having a sequence of SEQ ID NO: 616 and an HCDR2 having a sequence of SEQ ID NO: 617.

TABLE 6B
CD3 binding domains and sequences
Anti-CD3 binding domains - VHH (ABL)

| | |
|---|---|
| Anti-CD3 | EVQLVESGGGPVQAGGSLRLSCAASGRTYRGYSMAWFRQ |
| VHH | SPGKEREFVAAIVWSDGNTYYEDFVKGRFTISRDSAKNT |
| | LYLQMTNLKPEDTALYYCAAKIRPYIFKIAGQYDYWGQG |
| | TQVTVSS |
| | (SEQ ID NO: 615) |
| HCDR1 | GYSMA (SEQ ID NO: 616)(Kabat)/ |
| | GRTYRGYS (SEQ ID NO: 734)(IMGT) |
| HCDR2 | AIVWSDGNTYYEDFVKG (SEQ ID NO: 617) |
| | (Kabat)/IVWSDGNT(SEQ ID NO: 735)(IMGT) |
| HCDR3 | KIRPYIFKIAGQYDY (SEQ ID NO: 618) (Kabat)/ |
| | AAKIRPYIFKIAGQYDY (SEQ ID NO: 736)(IMGT) |

TABLE 6C
CD3 binding domains and sequences
Anti-CD3 binding domains-scFv (TR66)

| | |
|---|---|
| Anti CD3-scFv-2 | QVQLQQSGAELARPGASVKMSCKTSGYTFTRYTMHWVKQRPG |
| TR66 VH-VL | QGLEWIGYINPSRGYTNYNQKFKDKATLTTDKSSSTAYMQLSSL |
| | TSEDSAVYYCARYYDDHYSLDYWGQGTTLTVSSGGGGSGGGGS |
| | GGGGSGGGGSQIVLTQSPAIMSASPGEKVTMTCRASSSVSYMNW |
| | YQQKSGTSPKRWIYDTSKVASGVPYRFSGSGSGTSYSLTISSMEA |
| | EDAATYYCQQWSSNPLTFGAGTKLELK (SEQ ID NO: 669) |
| | (Underlining according to Kabat) |
| | According to Kabat numbering scheme, |
| | VH CDR1: RYTMH (SEQ ID NO: 673) |
| | VH CDR2: YINPSRGYTNYNQKFKD (SEQ ID NO: 674) |
| | VH CDR3: YYDDHYSLDY (SEQ ID NO: 717) |
| | VL CDR1: RASSSVSYMN (SEQ ID NO: 676) |
| | VL CDR2: DTSKVAS (SEQ ID NO: 677) |
| | VL CDR3: QQWSSNPLT (SEQ ID NO: 678) |
| | According to IMGT numbering scheme, |
| | VH CDR1: GYTFTRYT (SEQ ID NO: 718) |
| | VH CDR2: INPSRGYT (SEQ ID NO: 719) |
| | VH CDR3: ARYYDDHYSLDY (SEQ ID NO: 721) |
| | VL CDR1: SSVSY (SEQ ID NO: 880) |
| | VL CDR2: DTS (SEQ ID NO: 881) |
| | VL CDR3: QQWSSNPLT (SEQ ID NO: 882) |
| Anti-CD3-scFv-1 | QIVLTQSPAIMSASPGEKVTMTCRASSSVSYMNWYQQKSGTSPK |
| TR66 VL-VH | RWIYDTSKVASGVPYRFSGSGSGTSYSLTISSMEAEDAATYYCQQ |
| | WSSNPLTFGAGTKLELKGGGGSGGGGSGGGGSGGGGSGGGGSQ |
| | VQLQQSGAELARPGASVKMSCKTSGYTFTRYTMHWVKQRPGQ |
| | GLEWIGYINPSRGYTNYNQKFKDKATLTTDKSSSTAYMQLSSLTS |
| | EDSAVYYCARYYDDHYSLDYWGQGTTLTVSS (VL-VH). (SEQ ID |
| | NO: 670) |
| | (Underlined are CDR sequences according to Kabat |
| | numbering scheme) |
| | VH CDR1: GYTFTRYT (SEQ ID NO: 718) |
| | VH CDR2: INPSRGYT (SEQ ID NO: 719) |
| | VH CDR3: ARYYDDHYSLDY (SEQ ID NO: 721) |
| | VL CDR1: SSVSY (SEQ ID NO: 880) |
| | VL CDR2: DTS (SEQ ID NO: 881) |
| | VL CDR3: QQWSSNPLT (SEQ ID NO: 882) (IMGT numbering |
| | scheme) |
| Anti CD3-scFv | QVQLQQSGAELARPGASVKMSCKTSGYTFTRYTMHWVKQRPG |
| VH | QGLEWIGYINPSRGYTNYNQKFKDKATLTTDKSSSTAYMQLSSL |
| | TSEDSAVYYCARYYDDHYSLDYWGQGTTLTVSS (SEQ ID NO: |
| | 671) |
| Anti CD3-scFv | QIVLTQSPAIMSASPGEKVTMTCRASSSVSYMNWYQQKSGTSPK |
| VL | RWIYDTSKVASGVPYRFSGSGSGTSYSLTISSMEAEDAATYYCQQ |
| | WSSNPLTFGAGTKLELK (SEQ ID NO: 672) |
| HCDR1 | RYTMH (SEQ ID NO:673) |
| HCDR2 | YINPSRGYTNYNQKFKD (SEQ ID NO: 674) |
| HCDR3 | YYDDHYCLDY (Kabat, SEQ ID NO: 675 with unpaired cysteine at |
| | position 114 prior to modification); YYDDHYSLDY (Kabat, SEQ ID |
| | NO: 717), here, cysteine is replaced by serine. |
| LCDR1 | RASSSVSYMN (SEQ ID NO: 676) |
| LCDR2 | DTSKVAS (SEQ ID NO: 677) |
| LCDR3 | QQWSSNPLT (SEQ ID NO: 678) |

In one embodiment, an scFv for example, the scFv of SEQ ID NO: 669 or SEQ ID NO: 670 is designed from anti-CD3 antibody TR66. In one embodiment, anti-CD3 antibody sequence TR66 comprises the sequence of SEQ ID NO: 673. It was speculated that an unpaired cysteine residues occurring within the primary sequence of an Ig domain might interfere with the correct formation of the intrachain and/or interchain disulfide bounds which are essential for the proper folding and the stability of the resulting antibody fragment or not, several synthetic constructs were generated. Such unpaired cysteines might compromise efficacy, homogeneity, productivity and stability of the final protein product and should therefore be avoided. In addition to the “standard” set of cysteines involved in disulfide pairing free cysteine residues can be present in the variable domains. For example in the VH domain from the OKT3 antibody, three residues before the start of CDR-H3 a conserved cysteine at position H92 is present and forms a structural disulfide bond with position H22. But in this molecule at the position H100A (CDR-H3), another Cysteine (Cys) could allow mis-folding where H100A instead of H92 is involved in forming the disulfide bond with H22, thereby generating a mis-folded, insoluble and non-functional product. To overcome this possible mis-pairing of the cysteine residues, site directed substitution of the free cysteine was performed (Kipriyanov, Protein Engineering 10:445-453, 1997). By this single substitution a significant increase of productivity and stability of the scFv derived from OKT3 was achieved maintaining the overall binding activity. The VH domain of the anti-CD3 antibody TR66 (of SEQ ID NO: 679) prior to current modifications contains such a free cysteine at position 114 of the sequence as shown in SEQ ID NO: 669. Sequence comparison of the VH domain of the anti-CD3 antibody TR66 with the VH domain of the anti-CD3 antibody OKT3 shows 96.6% sequence homology.

A substitution of the free cysteine by a serine residue within the CDR-H3 of the VH domain of the anti-CD3 antibody TR66 was performed for bi-scFv proteins targeting CD3 This is reflected in SEQ ID NO: 669, or SEQ ID NO: 670. or SEQ ID NO: 672, or SEQ ID NO: 717. In some embodiments, the anti-CD3 antibody comprises the serine instead of cysteine (C114S) in the underlined position of HCDR3 under Kabat nomenclature YYDDHYSLDY (SEQ ID NO: 717).

In some embodiments, the cysteine is not substituted with serine. In some embodiments, the unsubstituted cysteine is evident in a sequence that has a HCDR3 sequence YYDDHYCLDY (SEQ ID NO: 675).

In some embodiments, the anti-CD3 binder is an scFv comprising an amino acid sequence that has at least 80% sequence identity to that of:

(SEQ ID NO: 725)
QVQLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSGGGGSGGGGSGGGGSGGGGSQIVLTQSP
AIMSASPGEKVTMTCRASSSVSYMNWYQQKSGTSPKRWIYDTSKVASGV
PYRFSGSGSGTSYSLTISSMEAEDAATYYCQQWSSNPLTFGAGTKLELK.

In some embodiments, the anti-CD3 binder is an scFv comprising an amino acid sequence that has at least 80% sequence identity to that of:

(SEQ ID NO: 669)
QVQLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYSLDYWGQGTTLTVSSGGGGSGGGGSGGGGSGGGGSQIVLTQSP
AIMSASPGEKVTMTCRASSSVSYMNWYQQKSGTSPKRWIYDTSKVASGV
PYRFSGSGSGTSYSLTISSMEAEDAATYYCQQWSSNPLTFGAGTKLELK.

This orientation is in the VH-VL configuration from N- to C-terminus. In some embodiments, the scFv comprises an amino acid sequence that is at least 85%, at least 90%, at least 91%, 92%, 93%, 94%, 95%, 96%, 97%, 98%, 99% identical to SEQ ID NO: 669. In some embodiments, the scFv comprises an amino acid sequence of SEQ ID NO: 669.

In some embodiments, the anti-CD3 binder is an scFv comprising an amino acid sequence that has at least 80% sequence identity to that of:

(SEQ ID NO: 670)
QIVLTQSPAIMSASPGEKVTMTCRASSSVSYMNWYQQKSGTSPKRWIY
DTSKVASGVPYRFSGSGSGTSYSLTISSMEAEDAATYYCQQWSSNPLT
FGAGTKLELKGGGGSGGGGSGGGGSGGGGSGGGGSQVQLQQSGAELAR
PGASVKMSCKTSGYTFTRYTMHWVKQRPGQGLEWIGYINPSRGYTNYN
QKFKDKATLTTDKSSSTAYMQLSSLTSEDSAVYYCARYYDDHYSLDYW
GQGTTLTVSS.

This orientation is in the VL-VH configuration from N- to C-terminus. In some embodiments, the scFv comprises an amino acid sequence that is at least 85%, at least 90%, at least 91%, 92%, 93%, 94%, 95%, 96%, 97%, 98%, 99% identical to SEQ ID NO: 670. In some embodiments, the scFv comprises an amino acid sequence of SEQ ID NO: 670.

In some embodiments, the CD3 binding domain comprises a heavy variable domain that is at least 80% identical, at least 85%, at least 90%, at least 91%, 92%, 93%, 94%, 95%, 96%, 97%, 98%, 99% identical to the sequence

(SEQ ID NO: 671)
QVQLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYSLDYWGQGTTLTVSS;
or
(SEQ ID NO: 726)
QVQLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSS.

In some embodiments, the CD3 binding domain comprises a light variable domain that is at least 80% identical, at least 85%, at least 90%, at least 91%, 92%, 93%, 94%, 95%, 96%, 97%, 98%, 99% identical to the sequence:

(SEQ ID NO: 672)
QIVLTQSPAIMSASPGEKVTMTCRASSSVSYMNWYQQKSGTSPKRWIY
DTSKVASGVPYRFSGSGSGTSYSLTISSMEAEDAATYYCQQWSSNPLT
FGAGTKLELK.

In some embodiments, the CD3 binder scFv can have the configuration: VH-linker-VL, having a sequence that is at least 8000 identical to the sequence:

(SEQ ID NO: 727)
QVQLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYSLDYWGQGTTLTVSSGGGGSGGGGSGGGGSGGGGSQIVLTQSPAIM
SASPGEKVTMTCRASSSVSYMNWYQQKSGTSPKRWIYDTSKVASGVPYRF
SGSGSGTSYSLTISSMEAEDAATYYCQQWSSNPLTFGAGTKLELK.

In some embodiments, the CD3 binder scFv can have the configuration: VL-linker-VH, having a sequence that is at least 800% identical to the sequence:

SEQ ID NO: 728)
QIVLTQSPAIMSASPGEKVTMTCRASSSVSYMNWYQQKSGTSPKRWIYD
TSKVASGVPYRFSGSGSGTSYSLTISSMEAEDAATYYCQQWSSNPLTFG
AGTKLELKGGGGSGGGGSGGGGSGGGGSGGGGSQVQLQQSGAELARPGA
SVKMSCKTSGYTFTRYTMHWVKQRPGQGLEWIGYINPSRGYTNYNQKFK
DKATLTTDKSSSTAYMQLSSLTSEDSAVYYCARYYDDHYSLDYWGQGTT
LTVSS.

In some embodiments, the CD3 binding domains comprise a HCDR1, 2, and 3 and an LCDR, 2, and 3 as follows according to Kabat and IMGT nomenclature as shown below:

TABLE 6D
CDR sequences and variations of TR66 antibody.

| Name | Numbering | CDR1 | CDR2 | CDR3 |
|---|---|---|---|---|
| VH(TR66) | IMGT | GYTFTRYT | INPSRGYT | ARYYDDHYCLDY |
| | | (SEQ ID NO: 718) | (SEQ ID NO: 719) | (SEQ ID NO: 720) |
| VH(TR66 | IMGT | GYTFTRYT | INPSRGYT | ARYYDDHYSLDY |
| (C114S)) | | (SEQ ID NO: 718) | (SEQ ID NO: 719) | (SEQ ID NO: 721) |
| VL(TR66) | IMGT | SSVSY | DTS | QQWSSNPLT |
| | | (SEQ ID NO: 722) | (SEQ ID NO: 723) | (SEQ ID NO: 724) |
| VL(TR66 | IMGT | SSVSY | DTS | QQWSSNPLT |
| (C114S)) | | (SEQ ID NO: 722) | (SEQ ID NO: 723) | (SEQ ID NO: 724) |
| VH(TR66) | Kabat | RYTMH | YINPSRGYTNYNQKFKD | YYDDHYCLDY |
| | | (SEQ ID NO: 673) | (SEQ ID NO: 674) | (SEQ ID NO: 675) |
| VH(TR66 | Kabat | RYTMH | YINPSRGYTNYNQKFKD | YYDDHYSLDY |
| (C114S)) | | (SEQ ID NO: 673) | (SEQ ID NO: 674) | (SEQ ID NO: 717) |
| VL(TR66) | Kabat | RASSSVSYMN | DTSKVAS | QQWSSNPLT |
| | | (SEQ ID NO: 676) | (SEQ ID NO: 677) | (SEQ ID NO: 678) |
| VL(TR66 | Kabat | RASSSVSYMN | DTSKVAS | QQWSSNPLT |
| (C114S)) | | (SEQ ID NO: 676) | (SEQ ID NO: 677) | (SEQ ID NO: 678) |

In some embodiments, the anti-CD3 binding domain
(scFv) comprises a sequence
(SEQ ID NO: 725)
QVQLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSGGGGSGGGGSGGGGSGGGGSQIVLTQSP
AIMSASPGEKVTMTCRASSSVSYMNWYQQKSGTSPKRWIYDTSKVASGV
PYRFSGSGSGTSYSLTISSMEAEDAATYYCQQWSSNPLTFGAGTKLELK.
In some embodiments, the anti-CD3 binding domain
(scFv) comprises a sequence
(SEQ ID NO: 728)
QIVLTQSPAIMSASPGEKVTMTCRASSSVSYMNWYQQKSGTSPKRWIYD
TSKVASGVPYRFSGSGSGTSYSLTISSMEAEDAATYYCQQWSSNPLTFG
AGTKLELKGGGGSGGGGSGGGGSGGGGSGGGGSQVQLQQSGAELARPGA
SVKMSCKTSGYTFTRYTMHWVKQRPGQGLEWIGYINPSRGYTNYNQKFK
DKATLTTDKSSSTAYMQLSSLTSEDSAVYYCARYYDDHYCLDYWGQGTT
LTVSS.
In some embodiments, the anti-CD3 binding domain
(scFv) comprises a sequence
(SEQ ID NO: 679)
KLQQSGAELARPGASVKMSCKTSGYTFTRYTMHWVKQRPGQGLEWIGYI
NPSRGYTNYNQKFKDKATLTTDKSSSTAYMQLSSLTSEDSAVYYCARYY
DDHYCLDYWGQGTTLTVSS.

In some embodiments, a composition is hereby provided for a bispecific sTCR or a multispecific sTCR, having a new anti-CD3 binder. In some embodiments, the anti-CD3 binder is an scFv. The anti-CD3 binder comprises a variable heavy chain domain (VH domain). The anti-CD3 binder comprises a variable light chain domain (VL domain).

TABLE 6E
CD3 binding domains and sequences
Anti-CD3 binding domains scFv 210112.

| | |
|---|---|
| Anti CD3-scFv | QVQLVQSGGGLVQPGGPLRLSCTASGFSFSNNYYMCWVRQAPG |
| 210112a | KGLEWVSCIYTNNIGSTWYASWAKGRFTISRDNSKNTLYLQMNS |
| (VH-VL) | LRAEDTAVYYCARGIYSDSSDFIGNLWGQGTLVTVSSGGGGSGG |
| | GGSGGGGSGGGGSAIQMTQSPSSLSASVGDRVTITCQASQTIYNY |
| | LAWYQQKPGKVPELLIYKASTLASGVPSRFMGTGFGTDFTLTIDS |
| | LQPEDAATYYCQQYNTIINVDRTFGGGTKVEIK |
| | (SEQ ID NO: 701) |
| Anti CD3-scFv | AIQMTQSPSSLSASVGDRVTITCQASQTIYNYLAWYQQKPGKVPE |
| 210112b (VL-VH) | LLIYKASTLASGVPSRFMGTGFGTDFTLTIDSLQPEDAATYYCQQ |
| | YNTIINVDRTFGGGTKVEIKGGGGSGGGGSGGGGSGGGGSGGGG |
| | SQVQLVQSGGGLVQPGGPLRLSCTASGFSFSNNYYMCWVRQAP |
| | GKGLEWVSCIYTNNIGSTWYASWAKGRFTISRDNSKNTLYLQM |
| | NSLRAEDTAVYYCARGIYSDSSDFIGNLWGQGTLVTVSS |
| | (SEQ ID NO: 702) |
| 210112 VH | QVQLVQSGGGLVQPGGPLRLSCTASGFSFSNNYYMCWVRQAPG |
| | KGLEWVSCIYTNNIGSTWYASWAKGRFTISRDNSKNTLYLQMNS |
| | LRAEDTAVYYCARGIYSDSSDFIGNLWGQGTLVTVSS |
| | (SEQ ID NO: 703) |
| 210112 VL | AIQMTQSPSSLSASVGDRVTITCQASQTIYNYLAWYQQKPGKVPE |
| | LLIYKASTLASGVPSRFMGTGFGTDFTLTIDSLQPEDAATYYCQQ |
| | YNTIINVDRTFGGGTKVEIK (SEQ ID NO: 704) |
| 210112 VH-CDR1 | NNYYMC (SEQ ID NO: 705) |
| (Kabat) | |
| 210112 VH-CDR2 | CIYTNNIGSTWYASWAKG (SEQ ID NO: 706) |
| (Kabat) | |
| 210112 VH-CDR3 | GIYSDSSDFIGNL (SEQ ID NO: 707) |
| (Kabat) | |
| 210112 VL-CDR1 | QASQTIYNYLA (SEQ ID NO: 708) |
| (Kabat) | |
| 210112 VL-CDR2 | KASTLAS (SEQ ID NO: 709) |
| (Kabat) | |
| 210112 VL-CDR3 | QQYNTIINVDRT (SEQ ID NO: 710) |
| (Kabat) | |
| 210112 VH-CDR1 | GFSFSNNYY (SEQ ID NO: 711) |
| (IMGT) | |
| 210112 VH-CDR2 | IYTNNIGST (SEQ ID NO: 712) |
| (IMGT) | |
| 210112 VH-CDR3 | ARGIYSDSSDFIGNL (SEQ ID NO: 713) |
| (IMGT) | |
| 210112 VL-CDR1 | QTIYNY (SEQ ID NO: 714) |
| (IMGT) | |
| 210112 VL-CDR2 | KAS (SEQ ID NO: 715) |
| (IMGT) | |
| 210112 VL-CDR3 | QQYNTIINVDRT (SEQ ID NO: 716) |
| (IMGT) | |

In one embodiment, the anti-CD3 binding domain comprises a heavy chain CDR3 sequence GIYSDSSDFIGNL (SEQ ID NO: 707). In some embodiments, the heavy chain CDR1 sequence of SEQ ID NO: 705. In some embodiments, the heavy chain CDR2 sequence of SEQ ID NO: 706. In one embodiment, the anti-CD3 binding domain comprises a light chain CDR3 sequence QQYNTIINVDRT (SEQ ID NO: 710). In one embodiment, the anti-CD3 binding domain comprises a light chain CDR1 sequence (SEQ ID NO: 708). In some embodiments, the light chain CDR2 sequence of NNYYMC (SEQ ID NO: 709).

In some embodiments, the anti-CD3 scFv comprises an anti-CD3 binding domain, wherein the binding domain comprises a heavy chain variable domain sequence of at least 60% of the sequence of SEQ ID NO: 703. In some embodiments, the heavy chain sequence has at least 70% sequence identity to SEQ ID NO: 703. In some embodiments, the heavy chain sequence has at least 75% sequence identity to SEQ ID NO: 703. In some embodiments, the heavy chain sequence has at least 80% sequence identity to SEQ ID NO: 703 In some embodiments, the heavy chain sequence has at least 85% sequence identity to SEQ ID NO: 703. In some embodiments, the heavy chain sequence has at least 90% sequence identity to SEQ ID NO: 703. In some embodiments, the heavy chain sequence has at least 91%, 92%, 93%, 94%, 95%, 96%, 97%, 98%, 99% sequence identity to SEQ ID NO: 703.

In some embodiments, the anti-CD3 scFv comprises an anti-CD3 binding domain, wherein the binding domain comprises a light chain variable domain sequence of at least 60% of the sequence of SEQ ID NO: 704. In some embodiments, the light chain variable domain sequence has at least 70% sequence identity to SEQ ID NO: 704. In some embodiments, the light chain variable domain sequence has at least 75% sequence identity to SEQ ID NO: 704. In some embodiments, the light chain variable domain sequence has at least 80% sequence identity to SEQ ID NO: 704. In some embodiments, the light chain variable domain sequence has at least 85% sequence identity to SEQ ID NO: 704. In some embodiments, the light chain variable domain sequence has at least 90% sequence identity to SEQ ID NO: 704. In some embodiments, the light chain variable domain sequence has at least 91%, 92%, 93%, 94%, 95%, 96%, 97%, 98%, 99% sequence identity to SEQ ID NO: 704.

In certain embodiments, the T cell engager comprises more than one binding domains for more than one T cell surface molecules, e.g., receptors. In some embodiments, a T cell engager comprises an scFv for binding a first receptor, which is expressed on a T cell, and a VHH for binding a second receptor, which is also expressed on the same T cell. An exemplary sTCR design may comprise a TCR engager comprising a first binding domain which is an anti-CD3 scFv, for example, and a second binding domain which is an anti-CD2 VHH, or vice versa. In some embodiments, the first binding domain may be an anti-CD3 VHH, and the second binding domain may be an anti-CD2 VHH. In some embodiments, the first binding domain of a T cell engager may be an anti-CD2 VHH and the second binding domain may be an anti CD3 VHH. In some embodiments, an exemplary CD2 binding domain is an anti-CD2 VHH, comprising a heavy chain variable domain having the sequence QVQLVESGGGLVQAGGSLRLSCAASGLTFSSYGMAWFRRALGREREFVGAIRPGTIPYYSE SVKGRFTVSKDNAKNTVSLQMNSLKPEDTAVYYCAAVRDYVGMPYYSGSAYEYWGQGT QVTVSS (SEQ ID NO: 623), or a sequence that is at least 80% identical to SEQ ID NO: 623. In some embodiments, the T cell engager comprises an anti-CD2 binding domain comprising a heavy chain variable region (VH) that comprises a heavy chain complementarity determining region 3 (HC CDR3) sequence of AAVRDYVGMPYYSGSAYEY (SEQ ID NO: 749) or VRDYVGMPYYSGSAYEY (SEQ ID NO: 750). In some embodiments, the VH of the anti-CD2 binding domain comprises a HC CDR2 sequence of IRPGTIP (SEQ ID NO: 751) or AIRPGTIPYYSESVKG (SEQ ID NO: 752). In some embodiments, the VH of the anti-CD2 binding domain comprises a HC CDR1 sequence of GLTFSSYG (SEQ ID NO: 753) or SYGMA (SEQ ID NO: 754).

In some embodiments, the anti-CD2 binding domain is a VHH domain. In some embodiments, the anti-CD2 binding domain comprises: a HC CDR1 sequence of GLTFSSYG (SEQ ID NO: 753), a HC CDR2 sequence of IRPGTIP (SEQ ID NO: 751), and a HC CDR3 sequence of AAVRDYVGMPYYSGSAYEY (SEQ ID NO: 749). In some embodiments, the anti-CD2 binding domain comprises: a HC CDR1 sequence of SYGMA (SEQ ID NO: 754), a HC CDR2 sequence of AIRPGTIPYYSESVKG (SEQ ID NO: 752), and a HC CDR3 sequence of VRDYVGMPYYSGSAYEY (SEQ ID NO: 750).

In some embodiments, a T cell engager component of the multispecific engager comprises a binding domain that can specifically bind to a T cell receptor constant domain. For example, a TCR alpha constant (TRAC) domain or a TCR beta constant domain (TRBC). An exemplary T cell engager comprises an anti-TRBC1 scFv domain, as an arm of the multispecific molecule, and may comprise the scFv known as JOVI-1 having the sequence: EVRLQQSGPDLIKPGASVKMSCKASGYTFTGYVMHWVYKQRPGQGLEWIGFINPYNDDIQ SNERFRGKATLTSDKSSTTAYMELSSLTSEDSAVYYCARGAGYNFDGAYRFEDFWGQGTT LTVSSGGGGSGGGGSGGGGSDVVMTQSPLSLPYSLGDQASISCRSSQRLVHSNGNTYLHWY LQKPGQSPKLLIYRVSNRFPGVPDRFSGSGSGTDFTLKISRVEAEDLGIYFCSQSTHVPYTFG GGTKLEIKR (SEQ ID NO: 624) or a fragment thereof. In some embodiments, the TRBC1 binding domain comprises a sequence that is at least 80% identical to the SEQ ID NO: 624. In some embodiments, the TRBC1 binding domain comprises a sequence that is at least 90% identical to SEQ ID NO: 624. In some embodiments the TRBC1 comprises at least 91%, 92%, 93%, 94%, 95%, 96%, 97%, 985, 99% sequence identity to SEQ ID NO: 624. In some embodiments, the TRBC1 binding domain comprises a heavy chain variable domain comprising a sequence: EVRLQQSGPDLIKPGASVKMSCKASGYTFTGYVMHWVYKQRPGQGLEWIGFINPYNDDIQ SNERFRGKATLTSDKSSTTAYMELSSLTSEDSAVYYCARGAGYNFDGAYRFEDFWGQGTT LTVSS (SEQ ID NO: 625), or a sequence that is at least 80% identical to SEQ ID NO: 625. In some embodiments, the anti-TRBC1 scFv domain comprises heavy chain CDRs delineated according to Kabat nomenclature are HCDR1: GYVMH (SEQ ID NO: 737), HCDR2: FINPYNDDIQSNERFRG (SEQ ID NO: 738); HCDR3: GAGYNFDGAYRFEDF (SEQ ID NO: 739); or according to IMGT nomenclature are HCDR1: GYTFTGYV (SEQ ID NO: 740), HCDR2: INPYNDDI (SEQ ID NO: 741); and HCDR3: ARGAGYNFDGAYRFEDF (SEQ ID NO: 742).

In some embodiments, the TRBC1 binding domain comprises a light chain variable domain comprising a sequence: DVVMTQSPLSLPYSLGDQASISCRSSQRLVHSNGNTYLHWYLQKPGQSPKLLIYRVSNRFPG VPDRFSGSGSGTDFTLKISRVEAEDLGIYFCSQSTHVPYTFGGGTKLEIKR (SEQ ID NO: 626), or a sequence that is at least 80% identical to SEQ ID NO: 626. In some embodiments, the anti-TRBC1 scFv domain comprises light chain CDRs delineated according to Kabat nomenclature are LCDR1: RSSQRLVHSNGNTYLH (SEQ ID NO: 743), LCDR2: RVSNRFP (SEQ ID NO: 744); LCDR3: SQSTHVPYT (SEQ ID NO: 745); or according to IMGT nomenclature are LCDR1: QRLVHSNGNTY (SEQ ID NO: 746), LCDR2: RVS (SEQ ID NO: 747); and LCDR3: SQSTHVPYT (SEQ ID NO: 745).

In some embodiments, the TRAC1 binding scFv comprises a sequence that is DVVMTQSPLSLPYSLGDQASISCRSSQRLVHSNGNTYLHWYLQKPGQSPKLLIYRVSNRFPG VPDRFSGSGSGTDFTLKISRVEAEDLGIYFCSQSTHVPYTFGGGTKLEIKRGGGGSGGGGSG GGGSEVRLQQSGPDLIKPGASVKMSCKASGYTFTGYVMHWVYKQRPGQGLEWIGFINPYN DDIQSNERFRGKATLTSDKSSTTAYMELSSLTSEDSAVYYCARGAGYNFDGAYRFEDFWGQ GTTLTVSS (SEQ ID NO: 748), or a sequence that is at least 80% identical to the sequence of SEQ ID NO: 748. The CDR sequences are: VH CDR 1: GYTFTGYV (SEQ ID NO: 740), VH CDR2: INPYNDDI (SEQ ID NO: 741). VH CDR3: ARGAGYNFDGAYRFEDF (SEQ ID NO: 742); VL CDR1: QRLVHSNGNTY (SEQ ID NO: 746), VL CDR2: RVS (SEQ ID NO: 747), VL CDR3: SQSTHVPYT (SEQ ID NO: 745) (IMGT).

In some embodiments, a T cell engager component of the multispecific engager comprises a binding domain that can specifically bind to CD8. In some embodiments, a T cell engager component of the multispecific engager comprises a binding domain that can specifically bind to CD8a. In some embodiments, a CD8a binding domain is an scFv. In some embodiments, the CD8a binding domain is a sdAb. In some embodiments, the CD8a binding domain is a VHH.

### Engineering Scaffold Designs for Tumor Cell Specific Multispecific Soluble TCR Engagers

Provided herein is a composition comprising a recombinant nucleic acid expressed in a mammalian cell, the recombinant nucleic acid comprising a sequence encoding a multispecific molecule, wherein the multispecific molecule is an engineered T cell receptor (TCR) construct, comprising: (i) an MHC-peptide complex engager comprising: (a) a T cell receptor (TCR) alpha variable (TRAV) domain, and (b) a TCR beta variable (TRBV) domain; and (ii) a T cell engager comprising one or more binding domains that bind to an extracellular domain of a receptor expressed by a T cell. In one aspect, provided herein is a composition comprising a recombinant nucleic acid comprising a sequence encoding a multispecific molecule, wherein the multispecific molecule comprises: (i) an MHC-peptide complex engager comprising: (a) a TCR alpha variable (TRAV) domain, and (b) a TCR beta variable (TRBV) domain; wherein the peptide in the MHC-peptide complex comprises a RAS peptide sequence; and (ii) a T cell engager comprising one or more binding domains that bind to an extracellular domain of a receptor expressed by a T cell. The multispecific T cell engagers lack any transmembrane domains.

In some embodiments, the multispecific molecule is soluble.

In some embodiments, the multispecific molecule lacks a trans-membrane domain.

In some embodiments, the multispecific molecule consists of two polypeptides, a first polypeptide and a second polypeptide. In some embodiments, the first polypeptide and the second polypeptide comprise one or more disulfide bonds between the two polypeptides. In some embodiments, the multispecific molecule comprises a first polypeptide and a second polypeptide, wherein the first polypeptide comprises the TRAV domain and the second polypeptide comprises the TRBV domain. In some embodiments, the peptide of the MHC-peptide complex is a peptide from a cancer antigen. In some embodiments, the peptide of the MHC-peptide complex is a mutant peptide, and wherein the MHC of the MHC-peptide complex binds the mutant peptide with a higher affinity compared to a corresponding wild-type peptide. In some embodiments, the MHC of the MHC-peptide complex comprises a class I MHC. In some embodiments, the MHC of the MHC-peptide complex is a class I MHC polypeptide. In some embodiments, the MHC of the MHC-peptide complex is encoded by an HLA that is represented in less than 1% of a human population. In some embodiments, the MHC of the MHC-peptide complex has a peptide binding affinity greater than at least 50 nM. In some embodiments, the one or more binding domains of the T cell engager bind to an extracellular domain of an endogenous receptor expressed by a T cell.

In one embodiment, the one or more binding domains of the T cell engager bind to an extracellular domain of a receptor selected from a group consisting of CD3, CD2, CD7, CD5, CD4, CD28, ICAM-1 and CD8. In some embodiments, the one or more binding domains of the T cell engager binds to CD3 delta, CD3 gamma or CD3 epsilon. In some embodiments, the one or more binding domains of the T cell engager comprises an antibody domain or antigen binding fragment thereof. In some embodiments, the binding domain of the T cell engager comprises an scFv or an sdAb. In some embodiments, the one or more binding domains of the T cell engager comprises a VHH.

In the scaffold designs contemplated herein, the one or more binding domains of the T cell engager comprises a first binding domain that binds to an extracellular domain of a first receptor expressed by a T cell and a second binding domain that binds to an extracellular domain of a second receptor expressed by a T cell. In some embodiments, the first binding domain of the T cell engager that binds to an extracellular domain of a first receptor expressed by a T cell comprises a first VHH, and the second binding domain of the T cell engager that binds to an extracellular domain of a second receptor expressed by a T cell comprises a second VHH.

In some embodiments, (i) the C-terminus of the first binding domain of the T cell engager is connected to the N-terminus of the second binding domain of the T cell engager; or (ii) the C-terminus of the first binding domain of the T cell engager is connected to the C-terminus of the second binding domain of the T cell engager. Alternatively, in some embodiments, (i) the N-terminus of the first binding domain of the T cell engager is connected to the N-terminus of the second binding domain of the T cell engager; or (ii) the N-terminus of the first binding domain of the T cell engager is connected to the C-terminus of the second binding domain of the T cell engager.

In some embodiments, the first binding domain of the T cell engager that binds to an extracellular domain of a first receptor expressed by a T cell comprises a scFv, and the second binding domain of the T cell engager that binds to an extracellular domain of a second receptor expressed by a T cell comprises a VHH. In some embodiments, the first binding domain that binds to an extracellular domain of the first receptor expressed by a T cell and the second binding domain that binds to an extracellular domain of the second receptor expressed by a T cell are connected by a peptide linker.

In some embodiments, the first binding domain binds to an extracellular domain of a first receptor expressed by a T cell selected from a group consisting of CD3, CD2, CD7, CD5, CD4, CD28, ICOS and CD8; and the second binding domain binds to an extracellular domain of a second receptor expressed by a T cell selected from a group consisting of CD3, CD2, CD7, CD5, CD4, CD28, ICOS and CD8. In some embodiments, the first receptor expressed by a T cell and the second receptor expressed by a T cell are different. In some embodiments, the first receptor expressed by a T cell is CD3 and the second receptor expressed by a T cell is CD2. In some embodiments, the first receptor expressed by a T cell is CD2 and the second receptor expressed by a T cell is CD3. In some embodiments, the first binding domain and the second binding domain of the T cell engager are configured to bind to the same receptor expressed by a T cell. In some embodiments, the T cell engager comprises an anti-CD3 binding domain. In some embodiments, the T cell engager comprises two more anti-CD3 binding domains. In some embodiments, the T cell engager comprises an anti-TRBC1 binding domain. In some embodiments, the T cell engager comprises an anti-CD2 binding domain. In some embodiments, the T cell engager comprises: (i) a first anti-CD3 binding domain, (ii) a second anti-CD3 binding domain, (iii) an anti-TRBC1 binding domain, (iv) an anti-CD2 binding domain, or (v) any combination of (i)-(iv).

The examples described in the previous sections can be variously utilized to design and build multispecific engagers, including modifications and variations such that one of skill in the art can easily conceptualize, for example by use of simple molecular biology skills. For example, VH and VL domain sequences within scFv may be taken up for redesigning into a sdAb, a VHH etc. Various technical considerations have been taken into account and utilized in some of the resulting structure, including, for example, swapping domains that are N terminal to a linker sequence to C terminal, with respect to the linker.

As an example Arndt et al. (Biochemistry 37:12918-12926, 1998) describe the so called domain swapping as a possible explanation for the appearance of non-covalently linked oligomers of scFv fragments. Under this model the protein state is subjected to a possible thermodynamic equilibrium between a monomeric and a dimeric/oligomeric form due to a constantly occurring intra- and intermolecular exchange of the VL/VH interface contacts. These oligomers could be present already in the cell culture supernatant and should be eliminated during the purification process. However these molecular species could be also formed during the storage of purified monomeric species. The preferred energetic status of the protein is strongly influenced by its overall design (primary sequence, linker length, VL/VH orientation etc.). Worn and Pltickthun (JMB 305:989-1010, 1999) mentioned that forms with higher content of monomeric species could be obtained by using a linker of 20 or more residues. Desplancq et al., (Protein Eng. 7:1027-1033, 1994) indicated that the variable domain orientation could also have an impact on the formation of dimers and high molecular forms. In the same publication Desplancq showed that a linker of 25 or 30 amino acids (aa) gave the best ratio of monomer over dimer for their particular antibody. The distance between the C-terminus of VL and the N-terminus of VH is around 39-43 A, and the distance between the C-terminus of VH and N-terminus of VL is 32-34 A (Pltickthun et al., From PCR to fermentation. (J. McCafferty, H. R. Hoogenboom, & D. J. Chriswell, Eds.). In: (IRL Press., pp. 203-252, 1996). To obtain similar molecular properties, a linker for the orientation VL-VH has to be longer than a VH-VL linker. Pltickthun et al., (From PCR to fermentation. (J. McCafferty, H. R. Hoogenboom, & D. J. Chriswell, Eds.). In: (IRL Press., pp. 203-252, 1996) recommended using linkers with a length of 15 or 20 amino acids in the orientation VH/VL and linkers with a length of 20 or 25 amino acids in the orientation VL/VH. Another possibility to force the formation of monomers and to stabilize the VH/VL domain interaction is to engineer an interface disulfide bond into the contact surface between the two domains. The introduction of a disulfide bridge at the position H44-L100 (Kabat numbering) has been the most frequently used in scFvs with satisfactory results (Brinkmann et al., PNAS. 90:7538-7542, 1993; Worn and Plockthun, Biochemistry 38: 8739-8750, 1999; Weatherill et al., PEDS. 25:321-329, 2012). This strategy has been used successfully to stabilize IgG-like bispecific antibodies combining scFv fused to full length IgG (Michaelson et al., mAbs 1: 128-141, 2009; Schanzer et al., Antimicrob. Agents. Chemother. 55:2369-2378, 2011). Weatherill et al., (PEDS 25:321-239, 2012) stabilized human scFvs (VH-(G4S)4-VL and VL-(G4S)4-VH) with a disulfide bond between the position VH44 and VL-100. Moreover this publication address the problem of possible domain swapping with scFv containing no interface disulfide bond by performing different SE-HPLC experiments at different load volume and concentration. The assays gave different results depending on the sample loading conditions for the non-stabilized scFv, but independent of the conditions used the disulfide stabilized molecules eluted like a monomer. Zhao et al., (Int. J. Mol. Sci. 12:1-11, 2011) introduced the same mutation in a scFv and observed higher stability of the stabilized molecule after storage for 20 h at 37 °C. For the bispecific format using scFv fused to full length IgG, Schanzer et al., (Antimicrob. Agents Chemother. 55:2369-2378 2011) compared the effect of the linker length and interface disulfide bond. They fused the parental scFv or scdFv (VH-(G4S)3-VL) either at the C- or N terminal part of the heavy chain or light chains. For the different linker length (20, 25 and 30 amino acids) they fused the parental scFv to the C-terminal part either of the heavy or light chains. The results obtained with the different linker length identified the 30 aa peptide as the more preferable linker for the production of stable monomers. The level of aggregates after 7 days storage at 40 °C was 50% for scFv15, 18% for scFv20, 8% for scFv25 and 6% for scFv30. But the disulfide scFv15 stabilized with the interface disulfide bond was slightly superior to the scFv30. The same approach was used by Michaelson et al., (mAbs, 1:128-141 2009), and they improved their parental IgG-like bispecific antibody containing scFv with 15 aa linker in VH/VL orientation (generating 40% aggregates), by increasing the linker length to 20 aa of the scFv and introducing the interface disulfide bond between the position VH44 and VL-100. The resulting molecule yielded more than 98% monomers that were stable after three months at 4 °C. The authors took the decision to work on the improvement of the scFv molecule before going to the bispecific format.

In the case of anti-RAS specific bispecific proteins as described in the disclosure, it is not known if the formation of dimers and high molecular forms could occur and what is the implication on the anti-RAS and/or the anti-CD3 scFv molecules. In order to assess an optimal overall molecule for the anti RAS specific bi-scFv protein for each separate scFv the following modifications are evaluated: domain orientation, linker length, introduction of an interface disulfide bond and a combination of the three modifications.

In another example, addition of cleavage sequences, such as autocleavable sequences for example, T2A, P2A F2A sequences between adjoining domains, within linkers, or between two amino acid chains to generate mature proteins is easily conceivable by one of skill in the art. An exemplary autocleavable sequence is T2A: TNFSLLKQAGDVEENPGP (SEQ ID NO: 627) Conjugating or inserting tag sequences, such as HIS tag at any region of the construct can be easily conceivable in the art. Functionalization of any of the linker sequences as known in the art can be achieved by one of ordinary skill in the art given the sequences and descriptions provided.

In some embodiments, described herein is a recombinant nucleic acid sequence encoding a first polypeptide, and a second polypeptide for a bispecific molecule, which comprises: (a) a nucleic acid sequence encoding a (a) TRAV/TRBV domains fused to a nucleic acid sequence encoding a TRAC/TRBC respectively; that binds to a cancer specific peptide in the MHC complex on the cancer cell, (b) a VHH domain that binds to a cell surface component of an effector cell, e.g. the T cell, (c) a short linker operably linking (a) and (b).

In some embodiments, nucleic acid sequences encoding the polypeptides comprising the VHH or scFv binding domains can be inserted in a suitable expression vector under one or more promoters, e.g. CMV at the 5′end, and a polyadenylation signal at the 3′-end of the sequences encoding the polypeptides.

In some embodiments, the constructs may comprise internal ribosomal entry site (IRES), e.g., a nucleic acid sequences encoding one or more polypeptides may be preceded by an IRES.

In some embodiments, the nucleic acid sequences encoding one of the polypeptides may be placed under a separate promoter control than the remaining of the expressed sequences.

In some embodiments, a trispecific engager is a fused construct of three binder domains, for example, in this case a p-MHC engager comprising at least a TRAV/TRBV domain specific to a cancer specific peptide in the MHC complex on the cancer cell, a first T cell engager arm—(e.g., a binding domain, an scFv or VHH) specific to a cell surface component on the T cell and a second T cell engager arm (a binding domain, e.g., an scFv or VHH)) to another cell surface component on the same T cell. In some embodiments, the trispecific engager is designed such that the cell surface component on the T cell to which the third binding domain can bind and provide an additional activation signal for the T cell to trigger killing of the target cell. In some embodiments the third binding domain binds to another T cell receptor on the T cell. In some embodiments, the third binding domain binds to a cytokine receptor which activates the receptor and triggers T cell intracellular signaling. In some embodiments, the third binding domain is CD3 or a fragment thereof.

In some embodiments, an multispecific molecule may comprise one or more linkers or spacers. Linkers or spacers may be made up of 2-50 amino acids. In some embodiments, a linker may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids. In some embodiments, the linker comprises 3-30 amino acid. In some embodiments, the linker is 4-20 or 5-10 amino acid long peptide. In some embodiments, the peptide linker is 5, 6, 7, 8, 9, 10, 11, 12, 13, 14, 15, 16, 17, 18, 19 or 20 amino acids in length. In some embodiments, a peptide linker or the two linker peptides with an anchor or a clasp together span a length of 50 amino acids or less, 45 amino acids or less, 40 amino acids or less, 35 amino acids or less, 30 amino acids or less, 25 amino acids or less, 20 amino acids or less, 15 amino acids or less, 10 amino acids or less, or 5 amino acids or less. In some embodiments, a peptide linker or the two linker peptides with an anchor or a clasp together span a length of 25 amino acids or less, 24 amino acids or less, 23 amino acids or less, 22 amino acids or less, 21 amino acids or less, 20 amino acids or less, 19 amino acids or less, 19 amino acids or less, 18 amino acids or less, 17 amino acids or less, 16 amino acids or less, 15 amino acids or less, 14 amino acids or less, 13 amino acids or less, 12 amino acids or less, 11 amino acids or less, 10 amino acids or less, 9 amino acids or less, 8 amino acids or less, 7 amino acids or less, 6 amino acids or less, or 5 amino acids or less.

In some embodiments, the linker may comprise one or more amino acids, wherein each amino acid is linked to the adjacent amino acid via natural peptide bonds, e.g., bond between amino group of one amino acid to the carboxyl group of a neighboring amino acid. In some embodiments, the antigen binding domains, scFvs or binding domains are linked with each other by a linker. In some embodiments, where there are more than one scFvs, the more than scFvs are linked with each other by linkers.

In some embodiments the linkers are flexible. In some embodiments the linkers comprise a hinge region. Linkers are usually short peptide sequences. In some embodiments the linkers are stretches of Glycine and one or more Serine residues. Other amino acids preferred for a peptide linker include but are not limited to threonine (Thr), serine (Ser), proline (Pro), glycine (Gly), aspartic acid (Asp), lysine (Lys), glutamine (Gln), asparagine (Asn), and alanine (Ala) arginine (Arg), phenylalanine (Phe), glutamic acid (Glu). Of these Pro, Thr, and Gln are frequently used amino acids for natural linkers. Pro is a unique amino acid with a cyclic side chain which causes a very restricted conformation. Pro-rich sequences are used as interdomain linkers, including the linker between the lipoyl and E3 binding domain in pyruvate dehydrogenase (GA₂PA₃PAKQEA₃PAPA₂KAEAPA₃PA₂KA) (SEQ ID NO: 628). Empirical linkers may be flexible linkers, rigid linkers, and cleavable linkers. Sequences such as (G4S)x (where x is multiple copies of the moiety, designated as 1, 2, 3, 4, and so on) comprise a flexible linker sequence. Other flexible sequences used herein include several repeats of glycine, e.g., (Gly)6 or (Gly)8. On the other hand, a rigid linker may be used, for example, a linker (EAAAK)x, where x is an integer, 1, 2, 3, 4 etc. gives rise to a rigid linker. In some embodiments, the linker or spacer may be made of nonreactive amino acid moieties, for example, a series of glycine or serine or alanine residues. An exemplary linker may comprise an amino acid sequence GSGS, or SGGG, or SGGGGSG (SEQ ID NO: 629). An exemplary linker may comprise an amino acid sequence SSGGGGSGGGGSGGGGS. (SEQ ID NO: 630).

The length of a linker peptide can be crucial in the design of a multispecific engager. For example, limiting the linker peptide length to less than 10 amino acids restricts the association between two adjacent domains. In some embodiments, the linker may comprise an anchoring or clasping function and may comprise a crosslinking moiety. The crosslinking moiety may be a peptide or a chemical crosslinking moiety.

In some embodiments, a peptide linker may further function as a conditionally cleavable linker. By conditionally cleavable it may be understood that the peptide is cleaved when the agent that cleaves it is available. By conditionally cleavable it may be understood that the peptide is cleaved when the agent that cleaves it is activated, for example if the agent that cleaves is an enzyme, is activated. By conditionally cleavable it may be understood that the peptide is cleaved when the agent that cleaves it is in an environment that allows activation of the agent; or the substance that activates it is present.

In some embodiments, a peptide linker may further function as a targeting peptide. Any one or more peptide linkers may comprise specialized functions, such as they can dimerize, trimerize or multimerize. In some embodiments, one or more linkers may comprise leucine zipper sequences.

In some embodiments the synthetic clasps of the linkers are non-peptide crosslinkers.

In some embodiments the complementary binding of cognate peptides with each other can be via chemical binding, such as crosslinking. Chemical crosslinkers can be useful for activating the crosslinking in vitro. There are homo- and heterobifunctional protein crosslinkers that can be commercially available. Examples include BS2G crosslinker (BS²G; Bis[Sulfosuccinimidyl]glutarate) is an amine-reactive, water soluble, homobifunctional protein crosslinker (both binding units at the opposite ends of a spacer arm have the identical reactive groups), or its membrane permeable version, DSG (Disuccinimidyl glutarate; Di(N-succinimidyl) glutarate); BS2G crosslinker (Bis[sulfosuccinimidyl] suberate; Sulfo-DSS; BSSS) or DST crosslinker (Disuccinimidyl tartrate), are among other homobifunctional crosslinkers for peptides; whereas BMPS (N-(β-Maleimidopropyloxy) succinimide ester; MBS crosslinker (m-Maleimidobenzoyl-N-hydroxysuccinimide ester); PDPH crosslinker (3-[2-Pyridyldithio]propionyl hydrazide) provide examples of some heterobifunctional crosslinkers.

In some embodiments, the variable light chain (V_{L}) subunit and the variable heavy chain (V_{H}) regions arranged in tandem within a multispecific engager may be linked via two linkers having the cognate peptide anchoring or clasping elements. The length of the linkers can limit or facilitate specific V_{L}-V_{H} associations. For example, limiting the linker peptide length to less than 10 amino acids restricts the association between two adjacent V_{L} and V_{H} domains.

In some embodiments, the anchoring or clasping elements exhibit an affinity having a K_{D}: less than 5×10⁻⁶M, or less than 10⁻⁶M, less than 5×10⁻⁷M, or less than 4×10⁻⁷M, or less than 3×10⁻⁷M, or less than 2×10⁻⁷M; or less than 10⁻⁷M, or less than 9×10⁻⁸M, or less than 8×10⁻⁸M, or less than 7×10⁻⁸M, or less than 6×10⁻⁸M, or less than 5×10⁻⁸M, or less than 4×10⁻⁸M, or less than 3×10⁻⁸M, or less than 2×10⁻⁸M, or less than 10⁻⁸M, or less than 10⁻⁸M, or less than 10⁻¹⁰M, or higher affinity.

Additionally, inclusion of the additional anchoring or hetero-multimerization domains in these higher order multispecific engagers (e.g., engagers with multiple binding domains) that are formed by the assembly of heterodimeric or hetero-multimeric units assist in the production, folding, stability and tissue availability of the multispecific engagers.

In some embodiments, the linker may be a flexible linker. For example, the linker may comprise amino acids that offer flexibility to the linker. Accordingly a flexible linker may allow necessary freedom of movement within the polypeptide for a binding domain to extend or recoil or twist and accommodate spatial limitations in binding the target molecule. A linker may link the TRAV with the TRAC. A linker may connect the TRAV or TRAC binder domains of a bi- or trispecific engager with the anti-TR binding domain. The linkers generally serve as structural elements that connect the effective binder sites. In some embodiments, the linker may be flexible. In some embodiments, the linker may be rigid. The length of the linker is adjusted as per the need of the design and the length that is optimal or necessary to space the binders at the opposite ends. In some embodiments, the linker may comprise a peptide that has a unique function, other than connecting two domains.

In one embodiment, provided herein is a T cell engager that binds to CD3 of a TCR complex on a T cell. The T cell engager is a anti CD3 scFv.

TRAV:TRBV interaction/stabilizing domains are components of the sTCR scaffolds designed herein. In some embodiments, the stabilizing domains are polypeptides connecting the TRAV and the TRBV individually, that help the TRAV:TRBV be positioned and aligned ideally for interacting with the antigen, e.g., the p-MHC complex. In some embodiments, the stabilizing domains comprise one or more polypeptides. In some embodiments, the TRAC and TRBC domains comprise dimerization and stabilization domains of the two polypeptides of the multispecific molecule. The TRAC and TRBC domains may comprise one or more engineered disulfide bridges connecting the two. For example, Thr 48 residue of TRAC and Ser 57 of TRBC1 or TRBC2 may be replaced by cysteine residues, the said cysteines forming a disulfide bond between the TRAC constant domain sequence and the TRBC1 or TRBC2 constant domain sequences of the polypeptides. In some embodiments, the ab heterodimeric TCRs (polypeptide comprising a TRAV domain and a TRAC domain, and polypeptide comprising a TRBV domain and a TRBC domain) described herein may comprise a TRAC constant domain sequence and a TRBC1 or TRBC2 constant domain sequences, and the TRAC constant domain sequence and the TRBC1 or TRBC2 constant domain sequence may be linked by the native disulfide bond between Cys4 of exon 2 of TRAC and Cys2 of exon 2 of TRBC1 or TRBC2, in addition to or in absence of the engineered disulfide bond as described above.

Also envisioned in the molecular design of bi- and/or trispecific molecules may comprise scaffold structures and helpers that assist in the engager's capability to modularly and concomitantly engage with multiple targets. These designs include additional anchoring or clasping elements for two or more binding domains and linkers and may promote and improve stability and flexibility in binding the multiple domains. The additional anchoring or clasping elements occur in cognate pairs, such that one of the cognate pair of the anchoring or clasping modality is attached to one of the binding domains of an engager and the other of the pair is attached to the other of the cognate pair.

In some embodiments, the engager is a recombinant protein comprising multiple binding domains as described throughout the specification, each having individual binding specificities, that are each linked together by linkers having cognate peptide anchoring or clasping elements that exhibit complementary binding with each other. For example, one binding domain of the recombinant protein is fused with the first of a pair of cognate peptides, and the other binding domain is fused with the second of the pair of peptides, wherein, the pair of peptides exhibit complementary binding with each other, wherein the pair of cognate peptides comprise leucine zipper domains that exhibit complementary binding with each other. For example, leucine zippers in naturally occurring protein-protein interactions, such as the zipper sequences within the binding regions of c-Fos and c-Jun proteins. In some embodiments, the pair of cognate peptides may comprise synthetic peptides designed to specifically bind to each other via synthetic clasps.

In some embodiments, the therapeutic agent is a recombinant protein comprising multiple binding fragments configured to facilitate accelerated association with each other by means of leucine zipper peptide pairs comprised in the recombinant proteins. Leucine zipper sequences often comprise a heptad leucine repeat and constitute adhesive peptide pairs when two peptides possess the leucine zipper structures. Among the naturally occurring leucine zippers, the c-Fos and c-Jun pairs are most widely known. They exhibit a strong binding affinity with K_{D}: 5.4×10⁻⁸ M. They form parallel coils. In some embodiments, the leucine zipper coil is the coil of the c-Fos: c-Jun pair.

Exemplary LZA leucine zipper domain may comprise an amino acid sequence as follows: AQLEKELQALEKENAQLEWELQALEKELAQK (SEQ ID NO: 631), or a sequence that has at least 80% sequence identity to SEQ ID NO: 631.

Exemplary LZB leucine zipper domain may comprise an amino acid sequence as follows: AQLKKKLQALKKKNAQLKWKLQALKKKLAQK, (SEQ ID NO: 632), or a sequence that has at least 80% sequence identity to (SEQ ID NO: 632).

In some embodiments, the anchoring or clasping elements exhibit specific heterodimerizing capabilities and do not exhibit homodimerization.

In some embodiments, the therapeutic agent is a recombinant protein comprising multiple binding fragments configured to facilitate accelerated association with each other by means of synthetic clasps. In some embodiments, the synthetic anchoring or clasping elements are designed to heterodimerize and prevent homodimerization.

In one aspect, provided herein are multispecific engagers, or multispecific engager molecules encoded by recombinant nucleic acids described herein. Multispecific engager molecules may be considered as polypeptides or proteins comprising two or more binding domains, or binders, for example a multispecific engager comprising two binders, wherein each binder binds to specific target, wherein each of the two binders may bind to a distinct target (in such case the engager is a bispecific engager). In some embodiments, the multispecific engager comprises three binders, each binding to a specific target distinct from one another, wherein such multispecific engager is a trispecific engager. In one embodiment, at least one engager of the multispecific engager disclosed herein is a T cell engager, that comprises a T cell specific binder, e.g., a binder that binds to a cell surface molecule expressed on a T cell. In some embodiments, the cell surface molecule is a receptor. In some embodiments, the cell surface molecule is a coreceptor. In some embodiments, the cell surface molecule is a ligand. In some embodiments, at least one engager of the multispecific engager is an engager that comprises a binding domain for a disease-specific epitope in complex with an MHC, expressed on a target cell. A target cell may be a cancer cell.

In some embodiments, provided herein are improvements in the binding domains and to increase the potency and effectiveness of bispecific T cell engagers (BiTEs) and/or trispecific T cell engagers (TRiTEs) not only as binders that anchor an effector cell, e.g., a T cell—with a target cell, e.g. a diseased cell, such as an infected cell or a tumor cell and juxtapose the two, but secondly, also to activate the T cell specifically to attack and lyse thereby destroy the target cell, and thirdly, in addition to the two above function, to activate an immune response cascade against the antigenic epitopes from the target cell. For this a robust primary response, including target cell killing by cytotoxicity, and cytokine and chemokine release by the effector T cell is necessary. Several improvements are designed to create highly effective BiTE and TRiTEs.

In one aspect, the BiTE or TRiTE comprises at least two polypeptides, for example, a first polypeptide and a second polypeptide. In some embodiments, the first polypeptide and the second polypeptide oligomerize in order to facilitate separate binding domains orient in a way such as to achieve the configuration required to bind a specific target molecule, e.g. an MHC-peptide complex.

In some embodiments oligomerization increases the likelihood of contacts with the effector and the target cells. In one embodiment, a naturally oligomerizing (e.g., dimerizing or trimerizing etc.) cellular protein or a fragment thereof can be incorporated in a BiTE or TRiTE polypeptide construct so as to facilitate oligomerization of the BiTE or TRiTEs units. Therefore, in one embodiment, a BiTE or a TRiTE of such design comprises a first monomer, comprising a first polypeptide comprising one or more binding domains (binders), of which at least one binder is a first binding domain capable of binding an antigen peptide in an MHC complex on a target cell only when the monomer is paired and correctly oriented with a second monomer comprising a second polypeptide comprising one or more binding domains, comprising a second binding domain that binds to the antigen peptide in an MHC complex. In some embodiments, the BiTE or TRiTE structures can comprise a protein or a fragment thereof that is capable of oligomerization (oligomerization domain). In some embodiments, the oligomerization domain can be fused to one or more binding domains. In one embodiment, a T cell engager is a domain that can bind to a T cell surface component, for example a receptor on a T cell. In one embodiment, the T cell engager domain is a ligand for a T cell surface receptor. In one embodiment, the T cell engager domain is an antibody or a fragment thereof that binds to a T cell surface receptor. In some embodiments, the T cell engager domain is an scFv that binds to a T cell surface receptor. In some embodiments, the T cell engager domain is an VHH domain that binds to a T cell surface receptor. In some embodiments, the T cell engager domain comprises one or more VHH domains, each of which binds to a T cell surface receptor. In some embodiments, the T cell engager domain comprises more than one VHH domains, each of which binds to a different T cell surface receptor expressed on the same T cell. In some embodiments, the more than one VHH domains may be on a single polypeptide chain and may be associated via a linker. The linker may be a peptide linker, for example, a flexible peptide linker comprising 2-100 amino acids.

In some embodiments, the multispecific molecule comprises a post-translational modification. In some embodiments, the multispecific molecule comprises a mammalian glycosylation signature. In some embodiments, the multispecific molecule is soluble and lacks a trans-membrane domain. In some embodiments, the multispecific molecule consists of two polypeptides, a first polypeptide and a second polypeptide.

With the intension of designing large libraries or repertoires of sTCRs into which the required CDRs and related specificities for antigen binding may be plugged in or exchanged to readily generate adequate molecules for a therapeutic applicability, Applicant designed a number of such TCRs that have the general structure and backbone designed and optimized for action with placeholder antigen-specificity (e.g., CDR) sequences which may be termed a scaffold. In essence a scaffold may be a soluble TCR design of the disclosure.

In one aspect, a recombinant nucleic acid encoding the sTCR scaffold may comprise a sequence encoding a single polypeptide, a TRAV domain, wherein the TRAV framework sequences are optimized, and the CDR1, CDR2, and CDR3 are interchangeable based on the target antigen, wherein the TRAV is operably linked with a sequence encoding a TRAC—which is in turn connected via a sequence encoding a post-translational auto-cleavage sequence, for example P2A, to a sequence encoding a polypeptide comprising the TRBC that is operably linked to a TRBV, wherein the TRBV framework sequences are optimized, and the CDR1, CDR2, and CDR3 are interchangeable based on the target antigen. Post translational cleavage leads to two polypeptides, which align with each other in such a way that the TRAV pairs with the TRBV forming the binding domain TRAV:TRBV specific for the antigen-MHC complex; and the TRAC and TRBC cross-react, which strengthens and stabilizes the TRAV:TRBV alignment. In some embodiments, a recombinant nucleic acid encoding the exemplary sTCR generated from the scaffold may comprise a sequence encoding a polypeptide, comprising, from N- to C-terminal: an N terminal native signal sequence; a first insert sequence comprising a TRAV that specifically binds to the desired antigen in association with a TRBV; a sequence for TRAC that constitutes a stabilizing domain in association with a TRBC comprised in the polypeptide chain; the TRAC may be further connected at the C terminal with an optional marker or tag peptide, for example a FLAG peptide, or any other functional domain; a P2A sequence; a native signal sequence, a second insert sequence comprising a TRBV sequence that specifically binds to the desired antigen in association with the TRAV in the polypeptide chain; a cleavage site; a sequence for TRBC stabilizing domain; which may be connected at the C terminal with an optional marker or tag peptide for example a hexa-HIS peptide or another functional domain. In some embodiments, a linker sequence comprising approximately 1-10 amino acids may be present between any two proximal domains. Upon post-translational cleavage, the mature protein may comprise self-assembled TRAV:TRBV domains on separate polypeptides, wherein the TRAV is operably linked with a TRAC, and the TRBV is operably linked with a TRBC, wherein the TRAC:TRBC form the stabilization domains. In some embodiments, the TRAC and the TRBC domains comprise 1, 2, 3, 4, 5, 6, 7, 8, 9, 10 or more point mutations conferring increased stabilization of the domains. In some embodiments the human TRAC comprises a disulfide lock position between T48C and T166C on the human TRAC one polypeptide domain. In some embodiments, the TRBC comprises disulfide lock positions S57C or S173C. In some embodiments, such a scaffold structure may be about 312 amino acids in length.

In one embodiment, an exemplary recombinant nucleic acid encoding an sTCR polypeptide uses a basic scaffold design as exemplified above but comprises leucine zipper stabilization domains for increased TRAV:TRBV stability. Accordingly, the recombinant nucleic acid comprises a sequence encoding a polypeptide comprising, from N- to C-terminus: an N terminal native signal sequence; a first insert sequence comprising a TRAV that specifically binds to the desired antigen in association with a TRBV; a TRAC sequence, optionally linked to a tag sequence (e.g., a FLAG tag); a sequence for a leucine zipper chain; a P2A sequence; a native signal sequence; a second insert sequence comprising the TRBV; a TRBC domain; a tag sequence, a leucine zipper sequence that oligomerizes with the other leucine zipper chain in the polypeptide, optionally connected at the C terminal with marker or tag peptide, for example a hexa-HIS tag, or another functional domain. The basic scaffold design of this type may comprise a 442 amino acid polypeptide.

In some embodiments, the scaffold comprises a human TRAC engineered to comprise one or more of stability enhancing mutations: S139F, T150I, A190T and the TRBC comprises E134K, H139R, D155P, S170D.

In some embodiments, the scaffold may comprise Fc domains in addition to the TCR constant regions for additional stabilization. In some embodiments the Fc domains comprise a knob-in-hole configuration. In some embodiments, the FcA domain comprises a T366W “knob” mutation. In some embodiments, the FcB domain comprises a T336S “hole” mutation. In some embodiments, the FcB domain comprises a L368A “hole” mutation. In some embodiments, the FcB domain comprises a Y407V “hole” mutation. In some embodiments, the scaffold comprises an Fc region from human IgG1. In some embodiments, the FcB comprises one or more amino acid substitutions, selected from M252E, I253A, and H435A substitutions.

In some embodiments, the scaffold comprises the basic configuration described in the previous paragraph, comprising an FcA-FcB domain in knob-and-hole configuration as described above, but lacks the TRAC and TRBC domains.

In some embodiments, the human TRAC may be linked via a short linker to a 10×His tag followed by a furin cleavage site. In some embodiments, the self-cleavable sequence may be F2A sequence; and the human TRBC sequence may be linked to a BAP tag.

The stabilization domains may comprise a disulfide bond between the human TRAC contributed by engineered cysteine on T166 (T166C) and human TRBC contributed by engineered cysteine at 5173 (S173C). A natural disulfide bond exists between TRAC (C213) and TRBC (C247). One or more glycosylation sites may be removed in TRAC and TRBC.

In some embodiments, the scaffold comprises a human TRAC engineered to comprise one or more of stability enhancing mutations: S139F, T150I, A190T and the TRBC comprises S170D, C191A or N205D. In some embodiments, both TRAC and TRBC sequences may comprise one or more N>Q substitutions.

The numbering of the amino acid residues in the TRAC and TRBC domains used herein follows the Kabat numbering system (Kabat, et al. Sequences of Immunological Interest Vol. 1 Fifth Edition 1991 US Department of Health and Human Services, Public Health Service, NIH).

In one embodiment, human TRAC may be fused to human IgG1 or a portion thereof, comprising the upper hinge of IgG1, the core hinge of IgG1 and the lower hinge of IgG1 followed by the Fc of IgG1. Accordingly, in one embodiment, the recombinant nucleic acid encoding this exemplary sTCR comprises a heavy chain (364 aa long) and a light chain (154 aa long) polypeptide; the heavy chain comprising, from N- to C-terminus: an N terminal native signal sequence; a first insert sequence comprising a TRAV that specifically binds to the desired antigen in association with a TRBV; a TRAC sequence that is fused at the C terminus to the upper hinge domain of human IgG1. A 10×-HIS tag may be attached to the C terminal end of the human IgG1. In some embodiments, the light chain comprises a signal sequence, a second insert sequence comprising the TRBV, operably linked to a human TRBC. In one embodiment, the human TRAC comprises one, two or three N>Q substitutions. In some embodiments one N>Q substitution is upstream of disulfide lock position 1 (T48C or T 166C), and two N>Q substitutions are downstream. The human IgG1 sequence has a disulfide site with TRBC C-terminal cysteine. In some embodiments the light chain comprises a disulfide lock position contributed by a S57C or S173C on the human TRBC sequence. In some embodiments, there are engineered disulfide bonds between IgG1 hinge region and human TRBC (C247).

In some embodiments, the dimerization or stabilization domains comprise SARAH domain A and SARAH domain B. In some embodiments, a recombinant nucleic acid encoding an engineered T cell receptor (TCR) construct, wherein the engineered TCR construct comprises (a) a first polypeptide comprising a TCR alpha variable (TRAV) domain comprising CDR1a, a CDR2a and a CDR3a; one or more TRAV framework region (TRAV-FR) sequences; (b) a second polypeptide comprising a TCR beta variable (TRBV) domain comprising CDR1b, CDR2b and a CDR3b; one or more TRBV framework region (TRBV-FR) sequences; wherein the first polypeptide and the second polypeptide recognize and bind to a first HLA-antigen complex, (c) a third polypeptide comprising a first alpha-helical dimerizing motif operably linked to the first polypeptide; wherein the third polypeptide is selected from a helical domain found in proteins involved in the Hippo signaling pathway, MST, RASSF and WW45; and (d) a fourth polypeptide comprising a second alpha-helical dimerizing motif that dimerizes with the third polypeptide forming an Fv-clasp. In some embodiments, the first alpha-helical dimerizing motif and the second alpha-helical dimerizing motif are heterologous. In some embodiments, the first alpha-helical dimerizing motif and the second alpha-helical dimerizing motif are homologous. In some embodiments, the first alpha-helical dimerizing motif and the second alpha-helical dimerizing motif are anti-parallel in orientation. In some embodiments, the first alpha-helical dimerizing motif is operably linked to the C terminus of the TRAV domain and the second alpha-helical dimerizing motif is operably linked to the C terminus of the TRBV domain. In some embodiments, the first alpha-helical dimerizing motif and the second alpha-helical dimerizing motif are fragments of a human Mst1 kinase. In some embodiments, the first alpha-helical dimerizing motif or the second alpha-helical dimerizing motif is a SARAH domain of the human Mst1 kinase or fragment thereof, wherein the first alpha-helical dimerizing motif is a SARAH A domain and the second alpha-helical dimerizing motif is a SARAH B domain. In some embodiments, the first polypeptide further comprises a partial TRAJ motif C-terminal to the TRAV domain, which is linked to the SARAH A domain via a first GS linker; and the second polypeptide further comprises a partial TRBJ motif C-terminal to the TRBV domain, which is linked to the SARAH B domain via a second GS linker.

In some embodiments an Fv clasp is utilized for strengthening the dimerization between TRAV and TRBV.

In some embodiments, the TRAV is engineered to include stability enhancing mutation comprising X96L, where X is any amino acid at position 96 of the TCR alpha variable domain as determined by IMGT numbering scheme. In some embodiments, the TRBV is engineered to include stability enhancing mutation comprising one or more of X9R and X10Y, wherein X is any amino acid at position 9 or 10 of the TCR beta variable domain as determined by IMGT numbering scheme.

In some embodiments the N-glycosylation sites may be removed from the TRAC and TRBC domains.

In some embodiments, one or more scaffold designs may comprise a single engineered disulfide bridge between TRAC (T166C) and TRBC (S173C), whereas the naturally occurring disulfide bond between TRAC (C213) and TRBC (C247) is removed by mutation of the cysteines to alanine or other amino acids.

In some embodiments, an exemplary scaffold may comprise stability enhancing TRAC and TRBC mutations: TRAC (S139F, T150I, A190T); TRBC (E134K, H139R, D155P, S170D); wherein the N-glycosylation sites are removed from the TRAC and TRBC. The scaffold may comprise an Fc fused to each polypeptide and may comprise both an engineered {TRAC (T166C) and TRBC (S173C)} and a natural {TRAC (C213) and TRBC (C247)} disulfide bridges. (Scaffold 14).

In some embodiments, the TRAV and TRBV framework regions are further optimized for stability, and efficient expression in mammalian cells. In some embodiments, the TRAV and TRBV are codon optimized.

One of the mutated RAS epitope-responsive TCR used in the studies herein for scaffold design is the Ros9a, having a TRAV amino acid sequence:

(SEQ ID NO: 619)
LAKTTQPISVDSYEGQEVNITCSHNNIATNDYITWYQQFPSQGPRFIIQG
YKTKVTNEVASLFIPADRKSSTLSLPRVSLSDTAVYYCLVGDMDQAGTAL
IFGKGTTLSVSSD;
wherein
CDR1:
(SEQ ID NO: 874)
NIATNDY
CDR2:
(SEQ ID NO: 875)
GYKTK
CDR3:
(SEQ ID NO: 876)
LVGDMDQAGTALI.
(CDR sequences according to IMGT).

The corresponding TRBV sequence is

(SEQ ID NO: 620)
AGVTQSPTHLIKTRGQQVTLRCSPKSGHDTVSWYQQALGQGPQFIFQ
YYEEEERQRGNFPDRFSGHQFPNYSSELNVNALLLGDSALYLCASSL
GEGRVDGYTFGSGTRLTVV;
wherein
CDR1:
(SEQ ID NO: 877)
SGHDT;
CDR2:
(SEQ ID NO: 878)
YYEEEE;
CDR3:
(SEQ ID NO: 879)
ASSLGEGRVDGYT.
(CDR sequences according to IMGT).

The TRAV:TRBV is capable of high affinity binding to KRAS G12D/C*08:02.

### Dimerization and Oligomerization, Stabilizing Domains.

In one aspect, the first polypeptide further comprises a dimerizing domain fused to the TRAV domain, and the second polypeptide comprises a dimerizing domain fused to the TRBV domain. In some embodiments, the first polypeptide comprises, from N to C terminus, the dimerizing domain of the first polypeptide, a peptide linker, and the TRAV domain, and the second polypeptide comprises, from N to C terminus, the dimerizing domain of the second polypeptide, a peptide linker, and the TRBV domain. In some embodiments, the dimerizing domain of the first polypeptide comprises a TCR alpha constant (TRAC) domain or portion thereof; and the dimerizing domain of the second polypeptide comprises a TCR beta constant (TRBC) domain or portion thereof. In some embodiments, the dimerizing domain of the first polypeptide and the dimerizing domain of the second polypeptide are linked by one or more disulfide bridges.

In some embodiments, wherein the dimerizing domain of the first polypeptide and the dimerizing domain of the second polypeptide are linked by a single disulfide bridge. In some embodiments, the first polypeptide comprises the T cell engager. In some embodiments, the T cell engager is connected to the dimerization domain of the first polypeptide. In some embodiments, the first polypeptide comprises, from N to C terminus, the T cell engager, the dimerization domain and the TRAV domain. In some embodiments, the first polypeptide comprises, from N to C terminus, the TRAV domain and the T cell engager. In some embodiments, the second polypeptide comprises the T cell engager. In some embodiments, the second polypeptide comprises, from N to C terminus, the T cell engager, the dimerizing domain and the TRBV domain. In some embodiments, the second polypeptide comprises, from N to C terminus, the TRBV domain and the T cell engager. In some embodiments, the T cell engager comprises an scFv that binds to the receptor expressed by a T cell, and wherein the polypeptide comprises, from N to C terminus, the scFv, the dimerization domain and the TRAV domain. In some embodiments, the T cell engager comprises an scFv that binds to the receptor expressed by a T cell and wherein the polypeptide comprises from N to C terminus, the dimerization domain, the TRAV domain and the scFv. In some embodiments, the T cell engager comprises an scFv that binds to the receptor expressed by a T cell and wherein the polypeptide comprises, from N to C terminus, the scFv, the dimerization domain and the TRBV domain. In some embodiments, the T cell engager comprises an scFv that binds to a receptor expressed by a T cell and wherein the polypeptide comprises from N to C terminus, the dimerization domain, the TRBV domain and the scFv. In some embodiments, the T cell engager comprises a VHH that binds to a receptor expressed by a T cell and wherein the polypeptide comprises, from N to C terminus, the VHH, the dimerization domain and the TRAV domain. In some embodiments, the T cell engager comprises a VHH that binds to a receptor expressed by a T cell and wherein the polypeptide comprises from N to C terminus, the dimerization domain, the TRAV domain and the VHH. In some embodiments, T cell engager comprises a VHH that binds to the receptor expressed by a T cell and wherein the polypeptide comprises, from N to C terminus, the VHH, the dimerization domain and the TRBV domain. In some embodiments, the T cell engager comprises a VHH that binds to the receptor expressed by a T cell and wherein the polypeptide comprises from N to C terminus, the dimerization domain, the TRBV domain and the VHH.

In some embodiments, the T cell engager comprises a first VHH domain and a second VHH domain configured to bind to the same receptor expressed by a T cell.

In some embodiments, wherein the T cell engager comprises a first VHH domain that binds the first receptor expressed by a T cell and the second VHH domain that binds the second receptor expressed by a T cell.

Therefore, provided herein is a composition comprising a recombinant polynucleic acid comprising a sequence encoding a first polypeptide chain comprising: a first binding domain that specifically interacts with an antigen peptide on a target cell, a first ligand or functional fragment thereof; and a first immunoglobulin (Ig) domain; wherein the first binding domain, the first ligand or functional fragment thereof, and the first Ig domain are operatively linked.

In some embodiments, the second binding domain specifically interacts with a first extracellular protein of a T cell. In some embodiments, the second ligand is configured to form a multimer with the first ligand. In some embodiments, the second ligand interacts with a second extracellular protein of a T cell, wherein the second extracellular protein is different than the first extracellular protein to which the second binding domain specifically interacts. In some embodiments, the first ligand interacts with a second extracellular protein of a T cell, wherein the second extracellular protein is different than the first extracellular protein to which the second binding domain specifically interacts. In some embodiments, the second polypeptide chain further comprises an additional binding domain that specifically interacts with an antigen peptide in the context of MHC complex on the target cell. In some embodiments, an additional binding domain of the second polypeptide chain specifically interacts with the antigen peptide of the target cell to which the first binding domain specifically interacts. In some embodiments, the second polypeptide chain further comprises a second Ig domain. In some embodiments, Ig domain is operatively linked to the second binding domain and the second ligand. In some embodiments, the additional binding domain of the first polypeptide chain specifically interacts with the first extracellular protein to which the second binding domain specifically interacts. In some embodiments, the first Ig domain and/or the second Ig domain comprises an Fc domain. In some embodiments, the first Ig domain and/or the second Ig domain is an IgG Fc domain. In some embodiments, the IgG domain is selected from the group consisting of IgG1, IgG4 and IgGA. The immunoglobulin chains or Fc domains selected for the BiTE or TRiTE typically form dimers. The Ig Fc chains may form a homodimer. Alternatively, the Ig Fc chains can be selected or configured to form a heterodimer. In one embodiment, the IgG polypeptide chain(s) is/are modified such that the modification helps to increase stability of the polypeptide. In some embodiments, the IgG polypeptide chain(s) is/are modified in order to increase flexibility of the polypeptide. In some embodiments, the modification is a point mutation. In some embodiments, the modification involves addition, deletion or altering of 1, 2, 3, 4, 5, 6, 7, 8, 9, 10 or more amino acids within the polypeptide. In some embodiments, the modification involves structurally altering the polypeptide.

For example, the Ig Fc chains are modified to incorporate a knob- and hole association between the two Fc chains. In some embodiments one or more amino acids may be suitably modified or altered to allow binding between adjacent polypeptide chains. Such modifications may include amino acid substitution of an amino acid with a cysteine, to introduce disulfide bonds between two adjacent polypeptides, or within the polypeptide. In some embodiments, the first Ig domain comprises a knob, e.g., wherein the knob comprises IgG1 Fc (F409R). In some embodiments, the second Ig domain comprises a hole wherein the hole comprises IgG1 Fc (K405L).

In some embodiments, any one of the exemplary scaffold designs can be used as modular platforms for one of skill in the art intending to generate soluble multispecific engager construct. The construct provided below is one of the basic structural motifs for s multispecific engager, and comprises the TCR-alpha and TCR beta chains and one or more tags (HIS tag and BAP tag), to which one or more T cell engagers may be conjugated to obtain a bi- or multispecific engager. In some embodiments, the scaffold comprises an engager molecule that is at least 70%, at least 80%, at least 90%, at least 91%, at least 92%, at least 93 at least 94%, at least 95%, at least 96%, at least 97%, at least 98%, at least 99%, identical to the sequence:

(SEQ ID NO: 633)
METDTLLLWVLLLWVPGSTGDLAKTTQPISVDSYEGQEVNITCSHNNIAT
NDYITWYQQFPSQGPRFIIQGYKTKVTNEVASLFIPADRKSSTLSLPRVS
LSDTAVYYCLVGDMDQAGTALIFGKGTTLSVSSDYIQNPDPAVYQLRDSK
SSDKFVCLFTDFDSQIQVSQSKDSDVYITDKCVLDMRSMDFKSNSAVAWS
QKSDFTCANAFQNSIIPEDTFFPSPESSCGSGHHHHHHHHHHRIRRSGSG
VKQTLNFDLLKLAGDVESNPGPMETDTLLLWVLLLWVPGSTGDDAGVTQS
PTHLIKTRGQQVTLRCSPKSGHDTVSWYQQALGQGPQFIFQYYEEEERQR
GNFPDRFSGHQFPNYSSELNVNALLLGDSALYLCASSLGEGRVDGYTFGS
GTRLTVVEDLKNVFPPEVAVFEPSKAEISRTQKATLVCLATGFYPPHVEL
SWWVNGKEVHDGVCTDPQPLKEQPALQDSRYALSSRLRVSATFWQDPRNH
FRCQVQFYGLSENDEWTQDRAKPVTQIVSAEAWGRADCGSGGLNDIFEAQ
KIEWHE;

wherein the TRAV sequence is:

(SEQ ID NO: 619)
LAKTTQPISVDSYEGQEVNITCSHNNIATNDYITWYQQFPSQGPRFII
QGYKTKVTNEVASLFIPADRKSSTLSLPRVSLSDTAVYYCLVGDMDQA
GTALIFGKGTTLSVSSD;

wherein the TRAC sequence is:

YIQNPDPAVYQLRDSKSSDKFVCLFTDFDSQIQVSQSKDSDVYITDKCV
LDMRSMDFKSNSAVAWSQKSDFTCANAFQNSIIPEDTFFPSPESS;

the TRBV sequence is: AGVTQSPTHLIKTRGQQVTLRCSPKSGHDTVSWYQQALGQGPQFIFQYYEEEERQRGNFPD RFSGHQFPNYSSELNVNALLLGDSALYLCASSLGEGRVDGYTFGSGTRLTVV (SEQ ID NO: 620); and the TRBC sequence is EDLKNVFPPEVAVFEPSKAEISRTQKATLVCLATGFYPPHVELSWWVNGKEVHDGVCTDPQ PLKEQPALQDSRYALSSRLRVSATFWQDPRNHFRCQVQFYGLSENDEWTQDRAKPVTQIVS AEAWGRADCGSGGLNDIFEAQKIEWHE. The TRAV and TRAC form the first polypeptide and the TRBV and the TRBC form the second polypeptide.

In some embodiments, the TRBV is N terminal to the TRAV. In some embodiments, the TRAV is N terminal with respect to TRBV. In some embodiments, the TRAV is associated with a tag, e.g., a HIS tag. In some embodiments the TRBV is associated with a tag, for example, a HIS tag or a BAP tag. In some embodiments, the TRAC is associated with a HIS tag or a BAP tag. In some embodiments, the TRAC is associated with a HIS tag or a BAP tag. In some embodiments, the TRAC is associated with a HIS tag or a BAP tag. In some embodiments, the TRBC is associated with a HIS-tag or a BAP tag.

In some embodiments, the alpha chain comprises one or more of a tag, a signal peptide a cleavage domain. In some embodiments, the beta chain comprises one or more of a tag, a signal peptide a cleavage domain.

In some embodiments, there is a cleavable sequence between the alpha chain and the beta chain. In some embodiments, the cleavable sequence is a P2A, F2A or T2A sequence.

In some embodiments, the scaffold comprises an engager molecule comprising a TRAV and a TRBV domain connected with a TRAC domain and a TRBC domain respectively; further comprising immunoglobulin chains conjugated to the TCR alpha and beta chains. An exemplary representative diagram is shown as Scaffold 5 in FIG. 3. In some embodiments, such a scaffold can comprise a sequence that is at least 70%, at least 80%, at least 90%, at least 91%, at least 92%, at least 93 at least 94%, at least 95%, at least 96%, at least 97%, at least 98%, at least 99%, identical to the sequence: METLLGVSLVILWLQLARVNSQQKEVEQNSGPLSVPEGAIASLNCTYSDRGSQSFFWYR QYSGKSPELIMSIYSNGDKEDGRFTAQLNKASQYVSLLIRDSQPSDSATYLCAGGGGAD GLTFGKGTHLIIQPYIQNPDPAVYQLRDSKSSDKFVCLFTDFDSQIQVSQSKDSDVYITDKC VLDMRSMDFKSNSAVAWSQKSDFTCANAFQNSIIPEDTFFPSPESSVEPKSCDKTHTCPPCP APELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYTLP PSRDELTKNQVSLTCLVKGFYPSDIAVEWESNGQPENNYKTTPPVLDSDGSFFLYSKLTVDK SRWQQGNVFSCSVMHEALHNHYTQKSLSLSPGKGSGHHHHHHHHH (Scaffold 5, 199.16 TCR as example having binding affinity for MART1/A*02:01) (SEQ ID NO: 634); and a corresponding beta chain can comprise a sequence that is at least 70%, at least 80%, at least 90%, at least 91%, at least 92%, at least 93 at least 94%, at least 95%, at least 96%, at least 97%, at least 98%, at least 99%, identical to the sequence:

(SEQ ID NO: 635)
MGTRLLCWAALCLLGAELTEAGITQSPKYLFRKEGQNVTLSCEQNLNHDA
MYWYRQDPGQGLRLIYYSQIVNDFQKGDIAEGYSVSREKKESFPLTVTSA
QKNPTAFYLCASSQGLAGAGELFFGEGSRLTVLEDLKNVFPPEVAVFEPS
KAEISRTQKATLVCLATGFYPPHVELSWWVNGKEVHDGVCTDPQPLKEQP
ALQDSRYALSSRLRVSATFWQDPRNHFRCQVQFYGLSENDEWTQDRAKPV
TQIVSAEAWGRADC

The TRAV sequence is marked as bold, and the TRBV sequence is marked by underline. The 10× Histidine tag, HHHHHHHHH (SEQ ID NO: 636) is conjugated at a terminus.

In some embodiments, the scaffold comprises an engager molecule comprising a TRAV and a TRBV domain; further comprising molecular clasp or linker between the two chains. In some embodiments, the molecular clasp is a Fv clasp (FIG. 3, scaffold 6). In some embodiments, the scaffold comprises an engager molecule comprising a TRAV, a TRBV and two stabilizer polypeptide segments connected via Fv clasp. In some embodiments, such a scaffold comprises a sequence:

(SEQ ID NO: 637)
MWWRLWWLLLLLLLLWPMVWAAQQKEVEQNSGPLSVPEGAIASLNCTYSD
RGSQSFFWYRQYSGKSPELIMSIYSNGDKEDGRFTAQLNKASQYVSLLIR
DSQPSDSATYLCAGGGGADGLTFGKGTHLIICPGSDYEFLKSWTVEDLQK
RLLALDPMMEQEIEEIRQKYQSKRQPILDAIEAKGSGHHHHHHHHHHRIR
RSGSGATNFSLLKQAGDVEENPGPMWWRLWWLLLLLLLLWPMVWAAGITQ
SPKYLFRKEGQNVTLSCEQNLNHDAMYWYRQDPGQGLRLIYYSQIVNDFQ
KGDIAEGYSVSREKKESFPLTVTSAQKNPTAFYLCASSQGLAGAGELFFG
EGSRLTVLEGSDYEFLKSWTVEDLQKRLLALDPMMEQEIEEIRQKYQCKR
QPILDAIEAKGSGSAWSHPQFEK.

In some embodiments, such a scaffold can comprise a sequence that is at least 70%, at least 80%, at least 90%, at least 91%, at least 92%, at least 93 at least 94%, at least 95%, at least 96%, at least 97%, at least 98%, at least 99%, identical to the sequence of SEQ ID NO: 637. Within the sequence, the TRAV sequence is denoted in bold, and TRBV sequence is denoted by underlining. Within this sequence, the sequence DYEFLKSWTVEDLQKRLLALDPMMEQEIEEIRQKYQCKRQPILDAIEAK (bold, italics) forms the Fv clasp (SEQ ID NO: 638).

In some embodiments, any one of the exemplary scaffold designs comprise an Fc fusion domain linked to the TCR chains, as shown in FIG. 3, scaffold 14. The Fc domains interlock with each other via a knob-in-hole structure for providing structural stability to the fused molecule. The exemplary scaffold 14 has a chain A, and a chain B. In some embodiments, such a scaffold can comprise a chain A having the sequence: MMGVKVLFALICIAVAEADLAKTTQPISVDSYEGQEVNITCSHNNIATNDYITWYQQFPS QGPRFIIQGYKTKVTNEVASLFIPADRKSSTLSLPRVSLSDTAVYYCLVGDMDQAGTAL IFGKGTTLSVSSDYIQNPDPAVYQLRDSKSSDKFVCLFTDFDSQIQVSQSKDSDVYITDK CVLDMRSMDFKSNSAVAWSQKSDFTCANAFQNSIIPEDTFFPSPESSCGGGGSGGGGSG GGGSDKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIEKTISK AKGQPREPQVYTLPPSREEMTKNQVSLWCLVKGFYPSDIAVEWESNGQPENNYKTTPPVLD SDGSFFLYSKLTVDKSRWQQGNVFSCSVMHEALHNHYTQKSLSLSPGKGGGGSHHHHHHH HHH (SEQ ID NO: 639). In some embodiments, such a scaffold can comprise a chain A sequence that is at least 70%, at least 80%, at least 90%, at least 91%, at least 92%, at least 93 at least 94%, at least 95%, at least 96%, at least 97%, at least 98%, at least 99%, identical to the sequence of SEQ ID NO: 639. Bold alphabets denote TRAV:TRAC fusion polypeptide. In some embodiments, such a scaffold can comprise a chain B having the sequence:

(SEQ ID NO: 640)
MMGVKVLFALICIAVAEADDAGVTQSPTHLIKTRGQQVTLRCSPKSGHD
TVSWYQQALGQGPQFIFQYYEEEERQRGNFPDRFSGHQFPNYSSELNVN
ALLLGDSALYLCASSLGEGRVDGYTFGSGTRLTVVEDLKNVFPPEVAVF
EPSKAEISRTQKATLVCLATGFYPPHVELSWWVNGKEVHDGVCTDPQPL
KEQPALQDSRYALSSRLRVSATFWQDPRNHFRCQVQFYGLSENDEWTQD
RAKPVTQIVSAEAWGRADCGGGGSGGGGSGGGGSDKTHTCPPCPAPELL
GGPSVFLFPPKPKDTLEASRTPEVTCVVVDVSHEDPEVKFNWYVDGVEV
HNAKTKPREEQYNSTYRVVSVLTVLHQDWLNGKEYKCKVSNKALPAPIE
KTISKAKGQPREPQVYTLPPSREEMTKNQVSLSCAVKGFYPSDIAVEWE
SNGQPENNYKTTPPVLDSDGSFFLVSKLTVDKSRWQQGNVFSCSVMHEA
LHNAYTQKSLSLSPGKGGGGSGLNDIFEAQKIEWHE.

In some embodiments, such a scaffold can comprise a chain B sequence that is at least 70%, at least 80%, at least 90%, at least 91%, at least 92%, at least 93 at least 94%, at least 95%, at least 96%, at least 97%, at least 98%, at least 99%, identical to the sequence of SEQ ID NO: 640. Underlined sequence is TRBV sequence; underlined and italicized, immunoglobulin sequence.

In some embodiments, any one of the exemplary scaffold designs described herein can be used as modular platforms for one of skill in the art intending to generate soluble multispecific engager construct. In some embodiments, the scaffold comprises an engager molecule that is at least 70%, at least 80%, at least 90%, at least 91%, at least 92%, at least 93 at least 94%, at least 95%, at least 96%, at least 97%, at least 98%, at least 99%, identical to the sequence set forth in SEQ ID NO: 641 (Ros9a Scaffold 19). Here the TCR alpha and beta chain variable domains are taken from TCRs that bind to RAS, having TRAV sequence as SEQ ID NO: 619 and TRBV sequence as SEQ ID NO: 620.

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence set forth in SEQ ID NO:642:

LAKTTQPISVDSYEGQEVNITCSHNNIATNDYITWYQQFPSQGPRFIIQG
YKTKVTNEVASLFIPADRKSSTLSLPRVSLSDTAVYYCLVGDMDQAGTAL
IFGKGTTLSVSSDYIQNPDPAVYQLRDSKSSDKFVCLFTDFDSQIQVSQS
KDSDVYITDKCVLDMRSMDFKSNSAVAWSQKSDFTCANAFQNSIIPEDTF
FPSPESSCGSGHHHHHHHHHHRIRRSGSGVKQTLNFDLLKLAGDVESNPG
PMMGVKVLFALICIAVAEAAIQMTQSPSSLSASVGDRVTITCRASQDIRN
YLNWYQQKPGKAPKLLIYYTSRLESGVPSRFSGSGSGTDYTLTISSLQPE
DFATYYCQQGNTLPWTFGQGTKVEIKGGGGSGGGGSGGGGSGGGGSGGGS
EVQLVESGGGLVQPGGSLRLSCAASGYSFTGYTMNWVRQAPGKGLEWVAL
INPYKGVSTYNQKFKDRFTISVDKSKNTAYLQMNSLRAEDTAVYYCARSG
YYGDSDWYFDVWGQGTLVTVSSGGGGSAGVTQSPTHLIKTRGQQVTLRCS
PKSGHDTVSWYQQALGQGPQFIFQYYEEEERQRGNFPDRFSGHQFPNYSS
ELNVNALLLGDSALYLCASSLGEGRVDGYTFGSGTRLTVVEDLKNVFPPE
VAVFEPSKAEISRTQKATLVCLATGFYPPHVELSWWVNGKEVHDGVCTDP
QPLKEQPALQDSRYALSSRLRVSATFWQDPRNHFRCQVQFYGLSENDEWT
QDRAKPVTQIVSAEAWGRADCGGGGSGLNDIFEAQKIEWHEGGGGSGGGG
SLPETGG
(Ros9a Scaffold 19, without signal peptide).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence set forth in SEQ ID NO: 643 (Ros9a Scaffold 20b).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence

(SEQ ID NO: 644)
AIQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFGQ
GTKVEIKGGGGSGGGGSGGGGSGGGGSGGGSEVQLVESGGGLVQPGGSLR
LSCAASGYSFTGYTMNWVRQAPGKGLEWVALINPYKGVSTYNQKFKDRFT
ISVDKSKNTAYLQMNSLRAEDTAVYYCARSGYYGDSDWYFDVWGQGTLVT
VSSGGGGSAGVTQSPTHLIKTRGQQVTLRCSPKSGHDTVSWYQQALGQGP
QFIFQYYEEEERQRGNFPDRFSGHQFPNYSSELNVNALLLGDSALYLCAS
SLGEGRVDGYTFGSGTRLTVVGSDYEFLKSWTVEDLQKRLLALDPMMEQE
IEEIRQKYQCKRQPILDAIEAKGSGHHHHHHHHHHRIRRSGSGATNFSLL
KQAGDVEENPGPMMGVKVLFALICIAVAEADLAKTTQPISVDSYEGQEVN
ITCSHNNIATNDYITWYQQFPSQGPRFIIQGYKTKVTNEVASLFIPADRK
SSTLSLPRVSLSDTAVYYCLVGDMDQAGTALIFGKGTTLSVCPDGSDYEF
LKSWTVEDLQKRLLALDPMMEQEIEEIRQKYQSKRQPILDAIEAKGSGSA
WSHPQFEK*
(Ros9a Scaffold 20b, without signal peptide)

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence having a polypeptide chain: (Ros9a Scaffold 21 Chain A) (SEQ ID NO: 645).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence having a polypeptide chain, (Ros9a Scaffold 21 Chain B) (SEQ ID NO: 646).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence set forth in SEQ ID NO: 647 (Ros9d Scaffold 19).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence:

(SEQ ID NO: 648)
LAKTTQPISVDSYEGQEVNITCSHNNIATNDYITWYQQFPSQGPRFIIQG
YKTKVTNEVASLFIPADRKSSTLSLPRVSLSDTAVYYCLVGDMDQAGTAL
IFGKGTTLSVSSDYIQNPDPAVYQLRDSKSSDKFVCLFTDFDSQIQVSQS
KDSDVYITDKCVLDMRSMDFKSNSAVAWSQKSDFTCANAFQNSIIPEDTF
FPSPESSCGSGHHHHHHHHHHRIRRSGSGVKQTLNFDLLKLAGDVESNPG
PMMGVKVLFALICIAVAEAAIQMTQSPSSLSASVGDRVTITCRASQDIRN
YLNWYQQKPGKAPKLLIYYTSRLESGVPSRFSGSGSGTDYTLTISSLQPE
DFATYYCQQGNTLPWTFGQGTKVEIKGGGGSGGGGSGGGGSGGGGSGGGS
EVQLVESGGGLVQPGGSLRLSCAASGYSFTGYTMNWVRQAPGKGLEWVAL
INPYKGVSTYNQKFKDRFTISVDKSKNTAYLQMNSLRAEDTAVYYCARSG
YYGDSDWYFDVWGQGTLVTVSSGGGGSAGVTQSPTHLIKTRGQQVTLRCS
PKSGHDTVSWYQQALGQGPQFIFQYYEEEERQRGNFPDRFSGHQFPNYSS
ELNVNALLLGDSALYLCASSLGQTNYGYTFGSGTRLTVVEDLKNVFPPEV
AVFEPSKAEISRTQKATLVCLATGFYPPHVELSWWVNGKEVHDGVCTDPQ
PLKEQPALQDSRYALSSRLRVSATFWQDPRNHFRCQVQFYGLSENDEWTQ
DRAKPVTQIVSAEAWGRADC.
(Ros9d Scaffold 19 without signal peptide)

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence having a polypeptide chain set forth in SEQ ID NO: 649 (Ros9d Scaffold 21 Chain A).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence having a polypeptide chain set forth in SEQ ID NO: 650 (Ros9d Scaffold 21 Chain B).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence set forth in SEQ ID NO: 651 (Ros10 Scaffold 19).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence:

(SEQ ID NO: 652)
QKEVEQNSGPLSVPEGAIASLNCTYSDRGSQSFFWYRQYSGKSPELIMFI
YSNGDKEDGRFTAQLNKASQYVSLLIRDSQPSDSATYLCAAAMDSSYKLI
FGSGTRLLVRPDYIQNPDPAVYQLRDSKSSDKFVCLFTDFDSQIQVSQSK
DSDVYITDKCVLDMRSMDFKSNSAVAWSQKSDFTCANAFQNSIIPEDTFF
PSPESSCGSGHHHHHHHHHHRIRRSGSGVKQTLNFDLLKLAGDVESNPGP
MMGVKVLFALICIAVAEAAIQMTQSPSSLSASVGDRVTITCRASQDIRNY
LNWYQQKPGKAPKLLIYYTSRLESGVPSRFSGSGSGTDYTLTISSLQPED
FATYYCQQGNTLPWTFGQGTKVEIKGGGGSGGGGSGGGGSGGGGSGGGSE
VQLVESGGGLVQPGGSLRLSCAASGYSFTGYTMNWVRQAPGKGLEWVALI
NPYKGVSTYNQKFKDRFTISVDKSKNTAYLQMNSLRAEDTAVYYCARSGY
YGDSDWYFDVWGQGTLVTVSSGGGGSAGITQSPRYKITETGRQVTLMCHQ
TWSHSYMFWYRQDLGHGLRLIYYSAAADITDKGEVPDGYVVSRSKTENFP
LTLESATRSQTSVYFCASSDPGTEAFFGQGTRLTVVEDLKNVFPPEVAVF
EPSKAEISRTQKATLVCLATGFYPPHVELSWWVNGKEVHDGVCTDPQPLK
EQPALQDSRYALSSRLRVSATFWQDPRNHFRCQVQFYGLSENDEWTQDRA
KPVTQIVSAEAWGRADCGGGGSGLNDIFEAQKIEWHEGGGGSGGGGSLPE
TGG
(Ros10 Scaffold 19, without signal peptide).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence: In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence:

MMGVKVLFALICIAVAEADLAKTTQPISVDSYEGQEVNITCSHNNIATND
YITWYQQFPSQGPRFIIQGYKTKVTNEVASLFIPADRKSSTLSLPRVSLS
DTAVYYCLVGDMDQAGTALIFGKGTTLSVSSDYIQNPDPAVYQLRDSKSS
DKFVCLFTDFDSQIQVSQSKDSDVYITDKCVLDMRSMDFKSNSAVAWSQK
SDFTCANAFQNSIIPEDTFFPSPESSCGSGHHHHHHHHHHRIRRSGSGVK
QTLNFDLLKLAGDVESNPGPMMGVKVLFALICIAVAEAQIVLTQSPAIMS
ASPGEKVTMTCRASSSVSYMNWYQQKSGTSPKRWIYDTSKVASGVPYRFS
GSGSGTSYSLTISSMEAEDAATYYCQQWSSNPLTFGAGTKLELKGGGGSG
GGGSGGGGSGGGGSGGGGSQVQLQQSGAELARPGASVKMSCKTSGYTFTR
YTMHWVKQRPGQGLEWIGYINPSRGYTNYNQKFKDKATLTTDKSSSTAYM
QLSSLTSEDSAVYYCARYYDDHYSLDYWGQGTTLTVSSGGGGSAGVTQSP
THLIKTRGQQVTLRCSPKSGHDTVSWYQQALGQGPQFIFQYYEEEERQRG
NFPDRFSGHQFPNYSSELNVNALLLGDSALYLCASSLGEGRVDGYTFGSG
TRLTVVEDLKNVFPPEVAVFEPSKAEISRTQKATLVCLATGFYPPHVELS
WWVNGKEVHDGVCTDPQPLKEQPALODSRYALSSRLRVSATFWQDPRNHF
RCQVQFYGLSENDEWTQDRAKPVTQIVSAEAWGRADCGGGGSGLNDIFEA
QKIEWHEGGGGSGGGGSLPETGG*
(Ros9a Scaffold 22).

In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence: In some embodiments, the scaffold comprises an engager molecule that is at least 70%, at least 80%, at least 85%, at least 90%, at least 91%, at least 92%, at least 93 at least 94%, at least 95%, at least 96%, at least 97%, at least 98%, or at least 99% identical to the sequence:

LAKTTQPISVDSYEGQEVNITCSHNNIATNDYITWYQQFPSQGPRFIIQG
YKTKVTNEVASLFIPADRKSSTLSLPRVSLSDTAVYYCLVGDMDQAGTAL
IFGKGTTLSVSSDYIQNPDPAVYQLRDSKSSDKFVCLFTDFDSQIQVSQS
KDSDVYITDKCVLDMRSMDFKSNSAVAWSQKSDFTCANAFQNSIIPEDTF
FPSPESSCGSGHHHHHHHHHHRIRRSGSGVKQTLNFDLLKLAGDVESNPG
PMMGVKVLFALICIAVAEAQIVLTQSPAIMSASPGEKVTMTCRASSSVSY
MNWYQQKSGTSPKRWIYDTSKVASGVPYRFSGSGSGTSYSLTISSMEAED
AATYYCQQWSSNPLTFGAGTKLELKGGGGSGGGGSGGGGSGGGGSGGGGS
QVQLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYSLDYWGQGTTLTVSSGGGGSAGVTQSPTHLIKTRGQQVTLRCSPKS
GHDTVSWYQQALGQGPQFIFQYYEEEERQRGNFPDRFSGHQFPNYSSELN
VNALLLGDSALYLCASSLGEGRVDGYTFGSGTRLTVVEDLKNVFPPEVAV
FEPSKAEISRTQKATLVCLATGFYPPHVELSWWVNGKEVHDGVCTDPQPL
KEQPALQDSRYALSSRLRVSATFWQDPRNHFRCQVQFYGLSENDEWTQDR
AKPVTQIVSAEAWGRADCGGGGSGLNDIFEAQKIEWHEGGGGSGGGGSLP
ETGG*
(Ros9a Scaffold 22, without signal peptide).

In some embodiments, provided herein is a polypeptide chain comprising a third binding domain that specifically interacts with an extracellular protein of the T cell. In some embodiments, the extracellular protein to which the third domain binding specifically interacts is different from the extracellular protein to which the second binding domain specifically interacts. In some embodiments, the extracellular protein to which the third domain binding specifically interacts are present on the same T cell. In some embodiments, the third binding domain is a CD8 molecule or a fragment thereof.

In one embodiment, the recombinant polynucleic acid is an mRNA.

In one aspect, provided herein is a cell comprising a polynucleic acid of any one of embodiments disclosed and described herein.

In one aspect, provided herein is pharmaceutical composition for use in treating cancer, comprising any one of the recombinant polynucleic acids, recombinant polypeptides or the cell of any one of embodiments disclosed and described herein.

In some embodiments, the antigen of the target cell is a cancer antigen peptide, a pathogenic antigen peptide or an autoimmune antigen peptide.

In one embodiment, the first binding domain, the second binding domain, the third binding domain, the additional binding domain of the first polypeptide chain, the additional binding domain of the second polypeptide chain and/or the additional binding domain of the third polypeptide chain comprises a TRAV/TRBV domain, a VH domain, a VL domain, a VNAR domain, a VHH domain, a single chain variable fragment (scFv), an Fab, a single-domain antibody (sdAb), a nanobody, a bispecific antibody or a diabody.

In any of the above embodiments, the BiTE or the TRiTE comprises any one or more of (i) an immunoglobulin Fc region connecting between two binding domains, (ii) a modified immunoglobulin Fc region connecting between two binding domains (e.g., with a knob and hole configuration), connects to a binding domain that binds to a different heterologous protein, e.g., a T cell expressing protein.

Methods for Preparing Multispecific T cell Engagers

The engagers described herein are produced as recombinant proteins. Generally, a polynucleotide sequence is constructed that encodes the recombinant protein is prepared and inserted into an expression vector, such as a plasmid, in proper orientation and correct reading frame for expression, if necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

The recombinant polynucleotide is synthesized by ligating DNA encoding, for example, a first binding domain, a linker, and a second binding domain in the same open reading frame using the molecular cloning techniques well known to one of skill in the art. In some embodiments, one or more polynucleotide sequences are arranged under the same promoter and regulatory elements for generation of a single polypeptide. In some embodiments, a short spacer may be inserted between two adjacent polynucleotides encoding two polypeptides wherein the spacer may encode a post translational cleavage site. The two polypeptides can be separated after translation by induction of the cleavage at the specific cleavage site. In some embodiments, the construct may be monocistronic or polycistronic. In some embodiments, more than one polypeptides are generated which then reassemble after translation. For example, light chain and heavy chain domains of an antibody or parts thereof can be generated by translation from two independent polynucleotide sequences, which are allowed to freely assemble with each other post-translationally. In another example, TRAV and TRBV of a TCR or parts thereof can be generated by translation from the two independent polynucleotide sequences. Alternatively, multiple polypeptide chains containing TRAV and TRBV variable domains that bind with each other are transcribed and translated from a single polynucleotide, which is cleaved after translation into respective peptide chains which can then reassemble. The polypeptide having a leader sequence is a preprotein and can have the leader sequence cleaved by the host cell to form the mature form of the polypeptide.

In some embodiments, the polynucleotide construct encodes an N terminal signal sequence upstream of the polypeptide for secretion of the polypeptides. In some embodiments, the N terminal signal sequence comprises a secretion sequence. The resulting translated protein product having the N terminal signal sequence for secretion would be secreted by the cell. In some embodiments, the mature protein lacks the signal peptide. Amino acid sequences of proteins designed with signal peptide at the N terminal therefore discloses the sequence of the mature protein, as the mature protein will assume the sequence of the protein as described without the signal protein sequence.

In some embodiments, signal peptide may have the amino acid sequence of any one of Table: 7.

TABLE 7
Exemplary N terminal signal peptide sequences.

| SEQUENCE | SEQ ID NO |
|---|---|
| METDTLLLWVLLLWVPGSTGD | 653 |
| METLLGVSLVILWLQLARVNS | 654 |
| MGTRLLCWAALCLLGAELTEA | 655 |
| MWWRLWWLLLLLLLLWPMVWAA | 666 |
| MMGVKVLFALICIAVAEAD | 667 |
| MMGVKVLFALICIAVAEA | 668 |

In some embodiments the plasmid vector is introduced or incorporated in the cell by known methods of transfection, such as using lipofectamine, or calcium phosphate, or via physical means such as electroporation or nucleofection. In some embodiments the viral vector is introduced or incorporated in the cell by infection, a process commonly known as viral transduction.

In some embodiments, recombinant nucleic acid is integrated or incorporated in an expression vector. A vector comprises one or more promoters, and other regulatory components, including enhancer binding sequence, initiation and terminal codons, a 5′UTR, a 3′UTR comprising a transcript stabilization element, optional conserved regulatory protein binding sequences and others.

In some embodiments the vectors of use in the application are specifically enhanced for expression. Other exemplary vectors of use throughout the process include phages, cosmids, or artificial chromosomes.

It is understood that any one of the first binder domains (domain binding to a target cell such as a cancer cell or a diseased cell or a pathogen) can be designed in combination with a second binder domain that binds to a T cell or a third binding domain described anywhere in the specification.

Viral Vectors: In some embodiments, the vector for expression of the recombinant protein is of a viral origin, namely a lentiviral vector or an adenoviral vector. In some embodiments, the nucleic acid encoding the recombinant nucleic acid is encoded by a lentiviral vector. In some embodiments the lentiviral vector is prepared in-house and manufactured in large scale for the purpose. In some embodiments, commercially available lentiviral vectors are utilized, as is known to one of skill in the art.

In some embodiments the viral vector is an Adeno-Associated Virus (AAV) vector.

Lipid nanoparticle mediated delivery: Lipid nanoparticles (LNP) may comprise a polar and or a nonpolar lipid. In some embodiments cholesterol is present in the LNPs for efficient delivery. LNPs are 100-300 nm in diameter provide efficient means of mRNA delivery to various cell types, including monocytes or macrophages. In some embodiments, LNP may be used to introduce the recombinant nucleic acids into a cell in in vitro cell culture. In some embodiments, the LNP encapsulates the nucleic acid wherein the nucleic acid is a naked DNA molecule. In some embodiments, the LNP encapsulates the nucleic acid wherein the nucleic acid is an mRNA molecule. In some embodiments, the LNP encapsulates the nucleic acid wherein the nucleic acid is inserted in a vector, such as a plasmid vector. In some embodiments, the LNP encapsulates the nucleic acid wherein the nucleic acid is a circRNA molecule.

In some embodiments, the LNP is used to deliver the nucleic acid into a subject. LNP can be used to deliver nucleic acid systemically in a subject. It can be delivered by injection. In some embodiments, the LNP comprising the nucleic acid is injected by intravenous route. In some embodiments the LNP is injected subcutaneously.

Microbubble mediated delivery: In some embodiments, microbubbles can be used for delivery of a composition comprising e.g., a nucleic acid, in a subject. Perfluorocarbon-filled microbubbles are stable for circulating in the vasculature as blood pool agents, they act as carriers of these agents until the site of interest is reached. Ultrasound applied over the skin surface can then be used to burst the microbubbles at this site, causing localized release of the drug. Various other forms of microbubbles include Sonazoid Optison, gas-filled albumin microbubble, and PESDA. Optimization of the composition of the microbubble with respect to the composition of the therapeutic agent that is delivered, along with the site of delivery intended is necessary.

In some embodiments, the recombinant proteins, for example the engagers, or the inflammatory proteins that are co-expressed, or any associated protein designed to be expressed in a T cell may be encoded by a recombinant nucleic acid, wherein the recombinant nucleic acid is an RNA. In some embodiments, the recombinant nucleic acid is an mRNA. In some embodiments, the mRNA comprises one or more modifications for enhanced expression and stability. In some embodiments, the mRNA may be circularized. In some embodiments, the modifications may include but are not limited to: replacement of a nucleobase with a base analog, or a modified nucleotide; inserting one or more motifs within the mRNA, and introducing modifications in the 5′- and 3′ UTRs. In some embodiments, the recombinant nucleic acid may be administered directly in a subject in need thereof.

### Pharmaceutical Composition

Provided herein is a pharmaceutical composition, comprising at least a first therapeutic agent which comprises multispecific molecule. The multispecific molecule in the composition may be in the form of peptides or polypeptides or a complex of multiple peptides. The multispecific molecule may be provided in a composition as purified recombinant proteins. The multispecific molecule may be provided in a composition as conjugated recombinant proteins, Vhh complexes, scFv complexes or nanobodies. The multispecific molecule may be in the form of a polynucleotide encoding the recombinant multispecific molecule. In some embodiments, polynucleotide encoding the multispecific molecule may comprise DNA, mRNA or circRNA or a liposomal composition of any one of these. The liposome is an LNP.

Pharmaceutical compositions can include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration.

Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

Acceptable carriers are physiologically acceptable to the administered patient and retain the therapeutic properties of the compounds with/in which it is administered. Acceptable carriers and their formulations are generally described in, for example, Remington' pharmaceutical Sciences (18^{th} ed. A. Gennaro, Mack Publishing Co., Easton, PA 1990). One example of carrier is physiological saline. A pharmaceutically acceptable carrier is a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Acceptable carriers are compatible with the other ingredients of the formulation and not injurious to a subject to whom it is administered. Nor should an acceptable carrier alter the specific activity of the neoantigens.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. Compositions can be formulated to be compatible with a particular route of administration (i.e., systemic or local). Thus, compositions include carriers, diluents, or excipients suitable for administration by various routes.

In some embodiments, a composition can further comprise an acceptable additive in order to improve the stability of immune cells in the composition. Acceptable additives may not alter the specific activity of the immune cells. Examples of acceptable additives include, but are not limited to, a sugar such as mannitol, sorbitol, glucose, xylitol, trehalose, sorbose, sucrose, galactose, dextran, dextrose, fructose, lactose and mixtures thereof. Acceptable additives can be combined with acceptable carriers and/or excipients such as dextrose. Alternatively, examples of acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage.

The pharmaceutical composition can be administered, for example, by injection. Compositions for injection include aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, and sodium chloride can be included in the composition. The resulting solutions can be packaged for use as is, or lyophilized; the lyophilized preparation can later be combined with a sterile solution prior to administration. For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed. Sterile injectable solutions can be prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation can be vacuum drying and freeze drying which yields a powder of the active ingredient plus any additional desired ingredient from a previously sterile-filtered solution thereof.

Compositions can be conventionally administered intravenously, such as by injection of a unit dose, for example. For injection, an active ingredient can be in the form of a parenterally acceptable aqueous solution which is substantially pyrogen-free and has suitable pH, isotonicity and stability. One can prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. Additionally, compositions can be administered via aerosolization.

When the compositions are considered for use in medicaments or any of the methods provided herein, it is contemplated that the composition can be substantially free of pyrogens such that the composition will not cause an inflammatory reaction or an unsafe allergic reaction when administered to a human patient. Testing compositions for pyrogens and preparing compositions substantially free of pyrogens are well understood to one or ordinary skill of the art and can be accomplished using commercially available kits.

Acceptable carriers can contain a compound that stabilizes, increases or delays absorption, or increases or delays clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (e.g., Fix (1996) Pharm Res. 13:1760 1764; Samanen (1996) J. Pharm. Pharmacol. 48:119 135; and U.S. Pat. No. 5,391,377).

The compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable, but are typified by an initial administration followed by repeated doses at one or more hour intervals by a subsequent injection or other administration. Alternatively, continuous intravenous infusions sufficient to maintain concentrations in the blood are contemplated.

### Treatment Methods

The instant disclosure comprises methods of treatment for diseases such as cancer and infection, where enhanced killing by T cells can be beneficial to remove diseased cells or infected cells. Unlike T cell-engaging bispecific antibodies (BsAbs), the parameters required for potent rTCR/anti-CD3 T cell redirection have been poorly characterized. Given the conserved nature of TCR-HLA-peptide interactions, it is speculated that the parameters dictating activity are likely generalizable, unlike BsAb-based T-cell engagers whose activity depends on both BsAb geometry and the membrane proximity of the targeted epitope.

Bispecific or any other multispecific engager TCRs described herein are directed to development of a therapeutic for activation of T cells in vivo at a cancer site. Exemplary compositions described here comprise a multispecific engager having a first engager comprising peptide-MHC binding domain, comprised of a TRAV:TRBV domain and a second engager, comprised of an anti-CD3 binding domain. Therapeutic components comprise a recombinant polynucleotide sequence encoding the multispecific sTCR. In some embodiments, the recombinant polynucleotide sequence encoding the multispecific sTCR is an mRNA. In some embodiments, the recombinant polynucleotide sequence encoding the multispecific sTCR is DNA, comprised as a plasmid vector. In some embodiments, the mRNA or the DNA plasmid vector is delivered a liposomal delivery vehicle or as a lipid nanoparticle encapsulated composition. In some embodiments, a viral delivery mechanism as is known to one skilled in the art is applied.

Cancer refers to diseases in which abnormal cells divide out of control and are able to invade other tissues. Cancer cells can spread to other parts of the body through the blood and lymph systems. Cancer can be characterized as a group of diseases involving abnormal cell growth that may begin in any tissue with the potential to invade or spread to other parts of the body. Some cancers can be characterized by their type, e.g., solid cancers, liquid cancers, or based on cellular origin such as hematopoietic cancers, osteosarcoma or lymphoma. Some cancers are known by the tissue of their origin or prevalence, e.g., endometrial cancers are characterized as cancers of the endometrial tissue. Some cancers are known by the organ or site of their origin or prevalence, e.g. lung cancer, head and neck cancer. Some cancers may be known by the overproductions of certain proteins, enzymes or biomarkers compared to their counterpart cells or tissues that are not cancerous. For example, certain proteins of viral origin may be associated with certain cancers, such as HPV-16 cancers, where certain proteins, for example HPV-16 E6 and E7 are overexpressed in cancer cells of this type. For example, certain antigens, such as KRAS may be highly expressed in certain cancer types, compared to non-cancer cells of the same type, and may be designated as KRAS overexpressing cancers. Typically, the overexpression of the antigen or the specific protein may be associated with or related to one or more mutations, and the cancer type may be associated with the mutation. For example mutation at the wild type G residue corresponding to position 12 in KRAS amino acid sequence may be mutated to V, D, C or other amino acids in KRAS-specific cancer cells. Certain specific antigens may be specifically expressed in cancer cells of certain cancer types, and not in other cancer types. Various cancer are contemplated herein that may not be restricted to a specific cell type, tissue type or organ, or even a certain stage of cancer. The TCRs of the present invention are directed to cancer cells that express a cancer antigen, that may be patient specific, which can be found during sequencing of a subject's genome from biological sample obtained from a cancer cell, cancer site or cancer tissue and compared to a corresponding non-cancer sample from the same subject; wherein the patient-specific antigen may be expressed in the cancer cell, and not on the non-cancer cell of the subject. In some cases, cancer antigens may be cancer specific, where the antigen is reportedly present in the type of cancer observed in multiple patients in the human population, who have been diagnosed of the specific cancer. In some cases, certain types are cancers are associated with an antigen, a protein (e.g., a viral protein) a gene mutation, All forms of cancer are contemplated herein.

In some embodiments, the cancer is a solid cancer. In some cases, the cancer is a liquid/blood cancer. The cancer can express or be diagnosed as expressing a tumor antigen. The tumor antigen can be a tumor-associated antigen or a tumor-specific antigen. In some cases, the cancer expresses a tumor-associated antigen (TAA). In some cases, the cancer expresses a tumor-specific antigen (TSA).

In some embodiments, the cancer is a cancer expressing or diagnosed as expressing a tumor-associated antigen (TAA).

The current classification of TAA can include the following group:
a) Cancer testis (CT) antigen: Since testis cells do not express HLA class I and class II molecules, these antigens may not be recognized by T cells in normal tissues and may therefore be immunologically considered tumor specific. Non-limiting examples of CT antigens include members of the MAGE family and NY-ESO-1;
b) Differentiation antigen: both tumor and normal tissue (from which the tumor originates) may contain TAAs. Differentiation antigens may be found, for example, in melanoma and normal melanocytes. Many of these melanocyte lineage-associated proteins may be involved in melanin biosynthesis and therefore these proteins may not tumor-specific, but may still widely be used for immunotherapy of cancer. Examples include, but are not limited to, tyrosinase for melanoma and PSA for Melan-A/MART-1 or prostate cancer;
c) Overexpressed TAA: gene-encoded widely expressed TAAs may be detected in histologically diverse tumors and in many normal tissues, with generally low expression levels. It is possible that many epitopes processed and potentially presented by normal tissues may be below the threshold level of T cell recognition, whereas their overexpression in tumor cells can trigger anticancer responses by breaking previously established tolerance. Non-limiting examples of such TAAs include Her-2/neu, survivin, telomerase or WTT;
d) tumor specific antigen can include unique TAAs resulted from mutations in normal genes (e.g., beta-catenin, CDK4). Some of these molecular changes can be associated with neoplastic transformation and/or progression. Tumor-specific antigens can generally induce strong immune responses without risking from the autoimmune response to normal tissue strips. On the other hand, these TAAs may only be associated with the exact tumor on which they are confirmed, and may not commonly shared among many individual tumors. In the case of tumor specific (related) isoform proteins, peptide tumor specificity (or relatedness) may also occur if the peptide is derived from tumor (related) exons;
e) TAA resulting from aberrant post-translational modification: such TAAs may result from proteins in the tumor that are neither specific nor overexpressed, but which still have tumor relevance (this relevance is due to posttranslational processing that is primarily active on tumors). Such TAAs may result from an altered glycosylation pattern, resulting in a tumor producing a novel epitope for MUC1 or in an event such as protein splicing during degradation, which may or may not be tumor specific; and
f) Tumor virus protein: these TTAs are viral proteins that may play a key role in the oncogenic process and, because they are foreign proteins (non-human proteins), may be able to trigger T cell responses. Non-limiting examples of such proteins include human papilloma type 16 viral proteins, E6 and E7, which are expressed in cervical cancer.

In some embodiments, the cancer is a cancer expressing or diagnosed as expressing a tumor-specific antigen (TSA).

Examples of tumor antigens include, but not limited to new antigens expressed during tumorigenesis, products of oncogenes and tumor suppressor genes, overexpressed or abnormally expressed intracellular proteins (e.g., HER2, MUC1, PSA, MUC1), carcinoembryonic antigen (CEA), tumor viruses (e.g., EBC, HPV, HBV, HCB, HTLV), cancer testis antigens (CTA) (e.g., MAGE family, NY-ESO), oncofetal antigens, altered surface glycolipids and glycoproteins, cell type-specific differentiation antigens (e.g., MART-1), or a derivative thereof. The tumor antigens can be selected from the group consisting of NY-ESO-1, Her2/neu, SSX-2, MAGE-C2, MAGE-A1, M-2433-233, MAGE-A10 254-262, KK-LC-1, p53, PRAME, Alpha fetoprotein, HPV6-E6, HPV16-E7, EBV-LMP1, RAS: G12D, RAS: G12C, RAS: G12A, RAS: G12S, RAS: G12R, RAS: G12R, RAS: G12R, RAS: G122 V, RAS: Q61H, RAS: Q61L, RAS: Q61R, RAS: G13D, TP53: V157G, TP53: V157F, TP53: R248Q, TP53: R248W, TP53: G245S, TP53: Y163C, TP53: G249S, TP53: Y240C, TP53: R175H, TP53: K132N, CDC73: Q254E, TPP2A6: N438Y, CTNN1: T41A, CTNNB1: S45P, CTNNB1: S37Y, CTNNB1: S33C, EGFR: L858R, EGFR: T790M, PIK3CA: E542K, PIK3CA: H1047R, GNAS: R201H, CDK4:R24, R24C H3. 3:K28M, BRAF: V600E, CHD4 K73Rfs, NRAS Q61R, IDH1:R132H, TVP23C: C51Y, and any combination thereof. The RAS can be KRAS, HRAS, or NRAS.

Other non-limiting examples of tumor-associated antigen or tumor-specific antigen includes antigens from Human Papilloma Virus, Epstein-Barr Virus, Merkel cell polyomavirus, Human Immunodeficiency Virus, Human T-cell Leukemia Virus, Human Herpes Virus 8, Hepatitis B virus, Hepatitis C virus, HCV, HBC, Cytomegalovirus, or from the group of single-point mutated antigens derived from the group consisting of the antigens of ctnnb1 gene, casp8 gene, HER2 gene, p53 gene, KRAS gene, NRAS gene, or particular tumor antigens issued or derived from the group consisting of RAS oncogene, BCR-ABL tumor antigens, ETV6-AML1 tumor antigens, melanoma-antigen encoding genes (MAGE), BAGE antigens, GAGE antigens, ssx antigens, ny-eso-1 antigens, cyclin-A1 tumor antigens, MART-1 antigen, gp100 antigen, CD19 antigen, prostate specific antigen, prostatic acidic phosphatase antigen, carcinoembryonic antigen, alphafetoprotein antigen, carcinoma antigen 125, mucin 16 antigen, mucin 1 antigen, human telomerase reverse transcriptase antigen, EGFR antigen, MOK antigen, RAGE-1 antigen, PRAME antigen, wild-type p53 antigen, oncogene ERBB2 antigen, sialyl-Tn tumor antigen, Wilms tumor 1 antigen, mesothelin antigen, carbohydrate antigens, B-catenin antigen, IUM-1 antigen, CDK4 antigen ERBB2IP antigen, and Melan-A melanoma tumor-associated antigen.

In some cases, the cancer cells express the tumor antigens, including and not limited to, NY-ESO-1, Her2/neu, SSX-2, MAGE-C2, MAGE-A1, M-2433-233, MAGE-A10 254-262, KK-LC-1, p53, PRAME, Alpha fetoprotein, HPV6-E6, HPV16-E7, EBV-LMP1, RAS: G12D, RAS: G12C, RAS: G12A, RAS: G12S, RAS: G12R, RAS: G12R, RAS: G12R, RAS: G122 V, RAS: Q61H, RAS: Q61L, RAS: Q61R, RAS: G13D, TP53: V157G, TP53: V157F, TP53: R248Q, TP53: R248W, TP53: G245S, TP53: Y163C, TP53: G249S, TP53: Y240C, TP53: R175H, TP53: K132N, CDC73: Q254E, TPP2A6: N438Y, CTNN1: T41A, CTNNB1: S45P, CTNNB1: S37Y, CTNNB1: S33C, EGFR: L858R, EGFR: T790M, PIK3CA: E542K, PIK3CA: H1047R, GNAS: R201H, CDK4:R24, R24C H3. 3:K28M, BRAF: V600E, CHD4 K73Rfs, NRAS Q61R, IDH1:R132H, or TVP23C: C51Y. The RAS can be KRAS, HRAS, or NRAS.

For example, cancers include, but are not limited to T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some embodiments, a composition comprising at least a first therapeutic agent, comprising a multispecific molecule, is administered per administration dose. In some embodiments, a composition comprising the therapeutic agent is administered in combination with a second or a third therapy.

In some embodiments, the composition comprising at least a first therapeutic agent comprising a multispecific molecule is administered once. In some embodiments, the composition comprising at least a first therapeutic agent is administered more than once. In some embodiments, the composition comprising at least a first therapeutic agent comprising a multispecific molecule is administered repeatedly, multiple times over a span of the therapy.

In some embodiments, the composition comprising at least a first therapeutic agent comprising a multispecific molecule is administered twice, thrice, four times, five times, six times, seven times, eight times, nine times, or ten times or more to a subject over a span of time comprising a few months, a year or more.

In some embodiments, the composition comprising at least a first therapeutic agent comprising a multispecific molecule is administered once weekly. In some embodiments, the composition comprising at least a first therapeutic agent comprising a multispecific molecule is administered twice weekly.

In some embodiments, the composition comprising at least a first therapeutic agent comprising a multispecific molecule is administered once every two weeks.

In some embodiments, the composition comprising at least a first therapeutic agent comprising a multispecific molecule is administered once every three weeks.

In some embodiments, the composition comprising at least a first therapeutic agent comprising a multispecific molecule is administered once monthly.

In some embodiments, the composition comprising at least a first therapeutic agent comprising a multispecific molecule is administered once in every 2 months, once in every 3 months, once in every 4 months, once in every 5 months or once in every 6 months.

In some embodiments, the composition comprising at least a therapeutic agent comprising a multispecific molecule is administered by injection.

In some embodiments, the composition comprising at least a therapeutic agent comprising a multispecific molecule is administered by infusion.

In some embodiments, the composition comprising at least a therapeutic agent comprising a multispecific molecule is administered by intravenous infusion.

In some embodiments, the composition comprising at least a therapeutic agent comprising a multispecific molecule is administered by subcutaneous infusion.

In some embodiments, treatment with multispecific molecule increase the T cell mediated target cell killing, compared to a case where no multispecific molecule was used. T cells retrieved from the tumor site after treatment with multispecific molecule may demonstrate a greater than 10%, or greater than 20%, or greater than 30%, or greater than 40%, or greater than 50%, or greater than 60%, or greater than 70%, or greater than 80%, or greater than 90%, or greater than 100%, or greater than 150%, or greater than 200%, or greater than 250%, or greater than 300%, or greater than 350%, or greater than 400%, or greater than 450%, or greater than 500%, or greater than 600%, or greater than 700%, or greater than 800%, or greater than 900%, or greater than 1000% increase in cytotoxicity with respect to a parallel case where no multispecific molecule was used.

In some embodiments, treatment with multispecific molecule increases cytokine production in associated T cells that may be retrieved from the tumor site, compared to a case with no multispecific molecule treatment. T cells retrieved from the tumor site after treatment with multispecific molecule may demonstrate a greater than 2-fold, or greater than 3-fold, or greater than 4-fold, or greater than 5-fold, or greater than 6-fold, or greater than 7-fold, or greater than 8-fold, or greater than 9-fold, or greater than 10-fold, or greater than 20-fold, or greater than 30-fold, or greater than 40-fold, or greater than 50-fold, or greater than 60-fold, or greater than 70-fold, or greater than 80-fold, or greater than 90-fold, or greater than 100-fold, or greater than 200-fold, or greater than 300-fold, or greater than 400-fold, or greater than 500-fold, or greater than 700-fold, or greater than 800-fold, or greater than 900-fold, or greater than 1000-fold increase in cytokine release compared to a case with no multispecific molecule treatment. The cytokines released may include IL-2, interferon gamma, or CD107.

In one aspect, provided herein is a pharmaceutical composition comprising the composition of any one of the embodiments comprising polynucleotides, polypeptides and oligomeric complexes described herein, and a pharmaceutically acceptable excipient. In some embodiments, the pharmaceutical composition further comprises a second therapeutic agent. Provided herein is a method of treating a disease or condition in a subject in need thereof, the method comprising administering to the subject the pharmaceutical composition described above. In some embodiments, administering the pharmaceutical composition comprises administering the pharmaceutical composition subcutaneously or intravenously. In some embodiments, the method further comprises administering a second therapeutic agent. In some embodiments, the disease or condition is a cancer. In some embodiments, the subject is human. In some embodiments, the subject has been treated for the disease or condition previously. In some embodiments, wherein the therapeutic agent binds to a receptor comprising an intracellular signaling domain that comprises a pro-inflammatory signaling domain. In some embodiments, the first therapeutic agent comprises a polypeptide that is less than 1000 amino acids or 1000 nm in length. In some embodiments, the first therapeutic agent comprises a polypeptide that is less than 500 amino acids or 500 nm in length. In some embodiments, the first therapeutic agent comprises a polypeptide that is 200-1000 amino acids or 200-1000 nm in length. In some embodiments, engagement of the binding domains of the first therapeutic agent contacts the cancer cell to the T cell. In some embodiments, the second binding domain specifically interacts with a T cell and promotes killing activity of the T cell. In some embodiments, the second binding domain specifically interacts with a T cell and promotes inflammatory signaling of the T cell. In some embodiments, the second binding domain specifically interacts with a T cell and inhibits anti-inflammatory activity of the T cell mediated by the target cell. In some embodiments, the second and/or the third binding domain promotes killing activity of the T cell. In some embodiments, the second and/or the third binding domain promotes inflammatory signaling of the T cell. In some embodiments, the second and/or the third binding domain inhibits anti-toxicity activity of the T cell mediated by the target cell. In some embodiments, the second and/or the third binding domain inhibits anti-inflammatory activity of the T cell mediated by the target cell. In some embodiments, the therapeutic agent comprises a therapeutic polypeptide. In some embodiments, the therapeutic agent comprises a recombinant nucleic acid encoding the therapeutic polypeptide. In some embodiments, the third binding domain or the additional therapeutic agent comprises a cytokine, a proinflammatory gene, a procaspase, or an anti-cancer agent. In some embodiments, the first binding domain, the second binding domain and the third binding domain bind to distinct non-identical target antigens. In some embodiments, the first binding domain, the second binding domain or the third binding domain is a ligand binding domain. In some embodiments, the first, the second or the third binding domains are operably linked by one or more linkers. In some embodiments, upon binding of the therapeutic agent to the T cell, the killing activity of the T cell is increased by at least 10%, or 20%, or 30%, or 40%, or 50%, or 60%, or 70% or 90% or 100% compared to a T cell not bound by the therapeutic agent. In some embodiments, engagement of the binding domains of first therapeutic agent triggers killing of the cancer cell by the T cell. In some embodiments, engagement of the additional therapeutic agent potentiates or increases the killing of the cancer cell by the T cell.

In some embodiments, a pharmaceutical composition comprising a polynucleic acid encoding any one or more of the polypeptides disclosed herein is described, wherein the composition further comprises a delivery vehicle. In some embodiments, the polynucleic acid is an mRNA.

In some embodiments, the polynucleic acid is DNA, comprised in an expression vector. In some embodiments, a nucleic acid may be delivered into a living system in the form of nanoparticles. Nucleic acid sequences of any one of the polypeptides disclosed herein may be delivered in vivo via suitable nanoparticles, e.g., liposomes, lipid nanoparticles, or polymeric nanoparticles. A lipid nanoparticle may comprise a polar lipid. In some embodiments, the lipid nanoparticle comprises a cationic lipid. In some embodiments, the lipid nanoparticle comprises a cationic lipid and a non-cationic lipid. In some embodiments, the lipid nanoparticle comprises a neutral lipid. In some embodiments, the lipid nanoparticle comprises a PEGylated lipid.

Alternatively, in some embodiments, the nucleic acid can be electroporated in a living cell ex vivo for preparation of a cellular therapy, wherein the cell is a T cell.

### EXAMPLES

### Example 1. Cloning sTCR and General Testing Methods

Cloning and characterization of the multispecific engagers are performed using general molecular biological and biochemical techniques. Generally, sTCR fusion proteins are produced in Expi293 suspension culture via transient transfection with an engineered pcDNA3.4 plasmid. The expressed protein is secreted into the culture media over the course of 6 days at a reduced temperature. After removing cells and cellular debris, secreted sTCR protein is isolated from the culture media via a series of protein purification steps including a combination of immobilized metal affinity chromatography (abbreviated as “IMAC”), protein-A/G affinity chromatography, ion exchange chromatography, and size exclusion chromatography. Functional assays are performed by co-incubation of sTCR: anti-CD3 bispecific proteins, target cells (pulsed with peptide epitope), and effector T cells combined with longitudinal tracking of target cell nuclear fluorescence using the Incucyte instrument.

In general, for the generation of bi-scFv IVT templates, plasmids were linearized downstream the poly(A)-tail using a class Iₜₛ endonuclease. Linearized template DNA was purified by phenol/chloroform extraction and sodium acetate precipitation as described elsewhere (Holtkamp, S. et al. (2006) Blood 108 (13), pp. 4009-4017). Linearized DNA templates were subjected to in vitro transcription using MEGAscript Kits (Ambion/Life Technologies, Darmstadt, Germany) according to the manufacturer's guidelines: pST1 templates were transcribed with the MEGAscript T7 Kit and pSFV templates with the MEGAscript SP6 Kit. For reactions with cap analog, the GTP concentration was lowered to 1.5 mM, and 6 mM of ARCA, beta-S-ARCA(D1), or beta-S-ARCA(D2), synthesized as described elsewhere (Grudzien, E. et al. (2004) RNA 10 (9), pp. 1479-1487; Kowalska, J. et al. (2008) RNA 14 (6), pp. 1119-1131; Stepinski, J. et al. (2001) RNA 7 (10), pp. 1486-1495) was added to the reaction. Purification of IVT-RNA was carried out with the MEGAclear Kit (Ambion/Life Technologies, Darmstadt, Germany) according to the manual. Concentration and quality of the IVT-RNA were assessed by spectrophotometry and analysis on a 2100 Bioanalyzer (Agilent Technologies, Santa Clara, CA, USA).

For the investigation of RNA translation into protein in mammalian cells the cell line BHK21 (ATCC CRL-13001) was frequently chosen as expression system. 2×107 BHK21 cells per ml were transfected by electroporation as described under example 16 with the difference that all steps were conducted at RT. 250 μl cell suspension was transferred to 0.4 cm Gene Pulser/MicroPulser Cuvettes (Bio-Rad, Dreieich, Germany) and 40 g/ml no.25 IVT-mRNA, 6RHU3 IVT-mRNA or 6RHU3 IVT-replicon RNA was added. Electroporation conditions were as follows: 300 V, 16 ms pulse length, 1 pulse, 400 ms interval length. Electroporated cells were resuspended in RT culture medium (RPMI1640, 10% FCS) and transferred into 15 cm culture dishes. 5 h after seeding, medium containing FCS was replaced by FCS-free medium. In case of examples 32 a and b, 18 h after electroporation cell culture supernatant and cells were harvested separately. Cell pellets were lysed in 1×LDS sample buffer (cat. no. NP0008; Life technologies, Darmstadt, Germany) and heated at 72 °C for 15 min. For ELISA analysis non-concentrated and approximately 50-fold concentrated supernatant was used. Concentration was carried out with Amicon ultra-15 centrifugal filter units (Merck Millipore, Billerica, MA, USA) according to the manufacturer's protocol. Supernatant is harvested 48 h post transfection and subjected to 40× concentration as described above. The constructs is tested for T cell activation by several ways. T cell proliferation in response to bispecific sTCR encoding IVT mRNA in the presence of target cells by flow cytometry is one measure of T cell activation and is a positive functional outcome for the sTCR construct.

Killing of target cells may be a measure of cytotoxicity of the T cells expressing bispecific sTCR, indicating that the T cells are activated and is a positive functional outcome for the sTCR construct.

Release of cytokines and chemokines by T cells expressing the bispecific sTCR in the presence of target cells is a measure of T cell activation and is a positive functional outcome for the sTCR construct.

The constructs may be tested for specificity and action using an in vitro antigen display system. The assay system can include naive Jurkat cell (or other T-cell derived cell line) transformed with a fluorescent reporter that is controlled by a transcription factor, which is itself triggered upon binding of a surface displayed pHLA. The pHLA construct to be introduced into the Jurkat cell line can comprise single chain trimer of HLA heavy chain, beta 2-microglobulin, and peptide epitope chains concatenated as one DNA construct—furthermore, the intracellular domain of the pHLA construct may contain co-stimulatory domains (e.g. CD28, CD3zeta) that lead to transcription factor activation upon pHLA binding at the cell surface. This cell based system could also be used to assess the toxicity and cross-reactivity of any sTCR protein-drug conjugate.

### Example 2. Bispecific sTCR Scaffold Designs

Construction of a bispecific sTCR molecule is performed by designing nucleic acid vectors encoding two polypeptides that dimerize post-translationally. The simplified cartoons in FIG. 2 show essentially four designs, with the MHC-peptide complex engager contributed by one domain of each polypeptide constituting the TRAV and the TRBV respectively, each fused to a dimerizing or stabilizing domain, contributed, for example by the TCR constant domains, a TRAC and a TRBC respectively that have natural as well as engineered cysteines forming disulfide bridges across the two polypeptides. The TCR engager is fused to the TRAV, or the TRBV, or the TRAC or the TRBC domains shown in four configurations, upper and lower left, upper and lower right respectively (FIG. 2). The figure in the middle shows a graphical representation of the molecule bound to an MHC-peptide complex. FIG. 3 depicts a diagrammatic representation of the various scaffold designed, and tested.

The following polypeptide scaffolds were generated and tested.

Ros9a Scaffold 19 (SEQ ID NO: 641).
The diagrammatic representation of the general
design and sequence is depicted in FIG. 7A.
The sequences of structural components from N terminal
to C terminal are represented as shown in Table 8A:
MMGVKVLFALICIAVAEADLAKTTQPISVDSYEGQEVNITCSHNNIATNDYITWYQQF
PSQGPRFIIQGYKTKVTNEVASLFIPADRKSSTLSLPRVSLSDTAVYYCLVGDMDQAG
TALIFGKGTTLSVSSDYIQNPDPAVYQLRDSKSSDKFVCLFTDFDSQIQVSQSKDSDVY
ITDKCVLDMRSMDFKSNSAVAWSQKSDFTCANAFQNSIIPEDTFFPSPESSCGSGHHH
HHHHHHHRIRRSGSGVKQTLNFDLLKLAGDVESNPGPMMGVKVLFALICIAVAEAAIQM
TQSPSSLSASVGDRVTITCRASQDIRNYLNWYQQKPGKAPKLLIYYTSRLESGVPSRFSGS
GSGTDYTLTISSLQPEDFATYYCQQGNTLPWTFGQGTKVEIKGGGGSGGGGSGGGGSGG
GGSGGGSEVQLVESGGGLVQPGGSLRLSCAASGYSFTGYTMNWVRQAPGKGLEWVALI
NPYKGVSTYNQKFKDRFTISVDKSKNTAYLQMNSLRAEDTAVYYCARSGYYGDSDWYF
DVWGQGTLVTVSSGGGGSAGVTQSPTHLIKTRGQQVTLRCSPKSGHDTVSWYQQALGQ
GPQFIFQYYEEEERQRGNFPDRFSGHQFPNYSSELNVNALLLGDSALYLCASSLGEGRVD
GYTFGSGTRLTVVEDLKNVFPPEVAVFEPSKAEISRTQKATLVCLATGFYPPHVELSWWV
NGKEVHDGVCTDPQPLKEQPALQDSRYALSSRLRVSATFWQDPRNHFRCQVQFYGLSEN
DEWTQDRAKPVTQIVSAEAWGRADCGGGGSGLNDIFEAQKIEWHEGGGGSGGGGSLPE
TGG (Ros9a Scaffold 19) (SEQ ID NO: 641).

| Domain/fragment | SEQUENCE | |
|---|---|---|
| N terminal signal | MMGVKVLFALICIAVAEAD (SEQ ID NO: 667) | |
| peptide | | |
| TCR alpha chain, | LAKTTQPISVDSYEGQEVNITCSHNNIATNDYITWYQQFPSQGPRFIIQ | |
| TRAV:TRAC | GYKTKVTNEVASLFIPADRKSSTLSLPRVSLSDTAVYYCLVGDMDQ | |
| fusion | AGTALIFGKGTTLSVSSDYIQNPDPAVYQLRDSKSSDKFVCLFTDFDS | |
| | QIQVSQSKDSDVYITDKCVLDMRSMDFKSNSAVAWSQKSDFTCANA | |
| | FQNSIIPEDTFFPSPESSC (SEQ ID NO: 680) | |
| | TRAV | LAKTTQPISVDSYEGQEVNITCSHNNIATNDYITWYQ |
| | | QFPSQGPRFIIQGYKTKVTNEVASLFIPADRKSSTLSLP |
| | | RVSLSDTAVYYCLVGDMDQAGTALIFGKGTTLSVSS |
| | | D (SEQ ID NO: 619) |
| | | CDR1: NIATNDY (SEQ ID NO: 874) |
| | | CDR2: GYKTK (SEQ ID NO: 875) |
| | | CDR3: LVGDMDQAGTALI (SEQ ID NO: 876) |
| | | (According to IMGT numbering scheme) |
| | TRAC | YIQNPDPAVYQLRDSKSSDKFVCLFTDFDSQIQVSQS |
| | | KDSDVYITDKCVLDMRSMDFKSNSAVAWSQKSDFT |
| | | CANAFQNSIIPEDTFFPSPESSC (SEQ ID NO: 681) |
| Linker, HIS-tag, | GSGHHHHHHHHHHRIRRSGSGVKQTLNFDLLKLAGDVESNPGP | |
| cleavable linker | (SEQ ID NO: 696) | |
| Signal peptide | MMGVKVLFALICIAVAEA (SEQ ID NO: 677) | |
| anti-CD3 VL | AIQMTQSPSSLSASVGDRVTITCRASQDIRNYLNWYQQKPGKAPKLL | |
| | IYYTSRLESGVPSRFSGSGSGTDYTLTISSLQPEDFATYYCQQGNTLP | |
| | WTFGQGTKVEIK (SEQ ID NO: 604) | |
| | LC CDRI QDIRNY (SEQ ID NO: 732), | |
| | LC CDR2 YTS (SEQ ID NO: 733), | |
| | LC CDR3 QQGNTLPWT (SEQ ID NO: 610). (IMGT numbering scheme) | |
| linker between | GGGGSGGGGSGGGGSGGGGSGGGS (SEQ ID NO: 682) | |
| anti-CD3 VL and | | |
| VH | | |
| anti-CD3 VH | EVQLVESGGGLVQPGGSLRLSCAASGYSFTGYTMNWVRQAPGKGL | |
| | EWVALINPYKGVSTYNQKFKDRFTISVDKSKNTAYLQMNSLRAEDT | |
| | AVYYCARSGYYGDSDWYFDVWGQGTLVTVSS (SEQ ID NO: 603) | |
| | HC CDR1 GYSFTGYT (SEQ ID NO: 729), | |
| | HC CDR2 INPYKGVS (SEQ ID NO: 730), | |
| | HC CDR3 ARSGYYGDSDWYFDV (SEQ ID NO: 731); (IMGT numbering | |
| | scheme) | |
| short linker | GGGGS (SEQ ID NO: 683) | |
| TCR beta chain- | AGVTQSPTHLIKTRGQQVTLRCSPKSGHDTVSWYQQALGQGPQFIFQ | |
| | YYEEEERQRGNFPDRFSGHQFPNYSSELNVNALLLGDSALYLCASSL | |
| | GEGRVDGYTFGSGTRLTVVEDLKNVFPPEVAVFEPSKAEISRTQKAT | |
| | LVCLATGFYPPHVELSWWVNGKEVHDGVCTDPQPLKEQPALQDSR | |
| | YALSSRLRVSATFWQDPRNHFRCQVQFYGLSENDEWTQDRAKPVT | |
| | QIVSAEAWGRADC (SEQ ID NO: 684) | |
| TRBV:TRBC | TRBV | AGVTQSPTHLIKTRGQQVTLRCSPKSGHDTVSWYQQ |
| fusion | | ALGQGPQFIFQYYEEEERQRGNFPDRFSGHQFPNYSS |
| | | ELNVNALLLGDSALYLCASSLGEGRVDGYTFGSGTR |
| | | LTVV (SEQ ID NO: 620) |
| | | CDR1: SGHDT (SEQ ID NO: 877) |
| | | CDR2: YYEEEE (SEQ ID NO: 878) |
| | | CDR3: ASSLGEGRVDGYT (SEQ ID NO: 879) |
| | | (Underlined, CDR sequences according to IMGT) |
| | TRBC | EDLKNVFPPEVAVFEPSKAEISRTQKATLVCLATGFY |
| | | PPHVELSWWVNGKEVHDGVCTDPQPLKEQPALQDS |
| | | RYALSSRLRVSATFWQDPRNHFRCQVQFYGLSENDE |
| | | WTQDRAKPVTQIVSAEAWGRADC (SEQ ID NO: 685) |

| | |
|---|---|
| linker-BAP- | GGGGSGLNDIFEAQKIEWHEGGGGSGGGGSLPETGG (SEQ ID NO: |
| LPETG tag | 686) |

Ros9a Scaffold 19, can also be without signal peptide (SEQ ID NO: 642) in its mature form.

Ros9a Scaffold 20b (SEQ ID NO: 643). The diagrammatic representation of the general design and sequence is depicted in FIG. 8A.

The above sequence (SEQ ID NO: 643) consists of the following
segments from N terminal to C terminal as shown in Table 8B:
MMGVKVLFALICIAVAEAAIQMTQSPSSLSASVGDRVTITCRASQDIRNYLNWYQQKPGK
APKLLIYYTSRLESGVPSRFSGSGSGTDYTLTISSLQPEDFATYYCQQGNTLPWTFGQGTK
VEIKGGGGSGGGGSGGGGSGGGGSGGGSEVQLVESGGGLVQPGGSLRLSCAASGYSFTG
YTMNWVRQAPGKGLEWVALINPYKGVSTYNQKFKDRFTISVDKSKNTAYLQMNSLRAE
DTAVYYCARSGYYGDSDWYFDVWGQGTLVTVSSGGGGSAGVTQSPTHLIKTRGQQVTL
RCSPKSGHDTVSWYQQALGQGPQFIFQYYEEEERQRGNFPDRFSGHQFPNYSSELNVNAL
LLGDSALYLCASSLGEGRVDGYTFGSGTRLTVVGSDYEFLKSWTVEDLQKRLLALDPMM
EQEIEEIRQKYQCKRQPILDAIEAKGSGHHHHHHHHHHRIRRSGSGATNFSLLKQAGDVE
ENPGPMMGVKVLFALICIAVAEADLAKTTQPISVDSYEGQEVNITCSHNNIATNDYITWY
QQFPSQGPRFIIQGYKTKVTNEVASLFIPADRKSSTLSLPRVSLSDTAVYYCLVGDMDQAG
TALIFGKGTTLSVCPDGSDYEFLKSWTVEDLQKRLLALDPMMEQEIEEIRQKYQSKRQPIL
DAIEAKGSGSAWSHPQFEK (Ros9a Scaffold 20b) (SEQ ID NO: 643).

| Domain/fragment | SEQUENCE |
|---|---|
| N terminal signal | MMGVKVLFALICIAVAEAD (SEQ ID NO: 667) |
| peptide | |
| Anti-CD3-VL | AIQMTQSPSSLSASVGDRVTITCRASQDIRNYLNWYQQKPGKAPKLL |
| | IYYTSRLESGVPSRFSGSGSGTDYTLTISSLQPEDFATYYCQQGNTLP |
| | WTFGQGTKVEIK (SEQ ID NO: 604) |
| | LC CDR1 QDIRNY (SEQ ID NO: 732), |
| | LC CDR2 YTS (SEQ ID NO: 733), |
| | LC CDR3 QQGNTLPWT (SEQ ID NO: 610). (IMGT numbering scheme) |
| Linker sequence | GGGGSGGGGSGGGGSGGGGSGGGS (SEQ ID NO: 682) |
| Anti-CD3 VH | EVQLVESGGGLVQPGGSLRLSCAASGYSFTGYTMNWVRQAPGKGL |
| | EWVALINPYKGVSTYNQKFKDRFTISVDKSKNTAYLQMNSLRAEDT |
| | AVYYCARSGYYGDSDWYFDVWGQGTLVTVSS (SEQ ID NO: 603) |
| | HC CDR1 : GYSFTGYT (SEQ ID NO: 729), |
| | HC CDR2 INPYKGVS (SEQ ID NO: 730), |
| | HC CDR3 ARSGYYGDSDWYFDV (SEQ ID NO: 731); |
| | (IMGT numbering scheme) |
| Short linker | GGGGS (SEQ ID NO: 683) |
| TCR beta chain, | AGVTQSPTHLIKTRGQQVTLRCSPKSGHDTVSWYQQALGQGPQFIFQ |
| TRBV | YYEEEERQRGNFPDRFSGHQFPNYSSELNVNALLLGDSALYLCASSL |
| | GEGRVDGYTFGSGTRLTVV (SEQ ID NO: 620) |
| | CDR1: SGHDT (SEQ ID NO: 877) |
| | CDR2: YYEEEE (SEQ ID NO: 878) |
| | CDR3: ASSLGEGRVDGYT (SEQ ID NO: 879) (IMGT numbering scheme) |
| Fv clasp domain | GSDYEFLKSWTVEDLQKRLLALDPMMEQEIEEIRQKYQCKRQPILD |
| | AIEAK (SEQ ID NO: 687) |
| short linker-HIS | GSGHHHHHHHHHHRIRRSGSGATNFSLLKQAGDVEENPGP (SEQ ID |
| tag, cleavable | NO: 688) |
| linker | |
| Signal peptide | MMGVKVLFALICIAVAEAD (SEQ ID NO: 667) |
| sequence | |
| TRAV sequence | LAKTTQPISVDSYEGQEVNITCSHNNIATNDYITWYQQFPSQGPRFIIQ |
| | GYKTKVTNEVASLFIPADRKSSTLSLPRVSLSDTAVYYCLVGDMDQ |
| | AGTALIFGKGTTLSV (SEQ ID NO:883) |
| TRAV sequence | LAKTTQPISVDSYEGQEVNITCSHNNIATNDYITWYQQFPSQGPRFIIQ |
| (alternate) | GYKTKVTNEVASLFIPADRKSSTLSLPRVSLSDTAVYYCLVGDMDQ |
| | AGTALIFGKGTTLSVSSD |
| Fv clasp domain | CPDGSDYEFLKSWTVEDLQKRLLALDPMMEQEIEEIRQKYQSKRQPI |
| | LDAIEAKGSGSAWSHPQFEK (SEQ ID NO: 689). |

The polypeptide Ros9a Scaffold 20b can also be without signal peptide and having the SEQ ID NO: 644.

Ros9a Scaffold 21. The diagrammatic representation of the general design and sequence is depicted in FIG. 9A. The first polypeptide chain (Chain A, SEQ ID NO: 645) and the second polypeptide chain (Chain B, SEQ ID NO: 646) were generated. These polypeptides comprise sequences set forth below:

Chain A:
(SEQ ID NO: 645)
MMGVKVLFALICIAVAEADLAKTTQPISVDSYEGQEVNITCSHNN
IATNDYITWYQQFPSQGPRFIIQGYKTKVTNEVASLFIPADRKSS
TLSLPRVSLSDTAVYYCLVGDMDQAGTALIFGKGTTLSVSSDYIQ
NPDPAVYQLRDSKSSDKFVCLFTDFDSQIQVSQSKDSDVYITDKC
VLDMRSMDFKSNSAVAWSQKSDFTCANAFQNSIIPEDTFFPSPES
SCGGGGSGGGGSGGGGSDKTHTCPPCPAPELLGGPSVFLFPPKPK
DTLMISRTPEVTCVVVDVSHEDPEVKFNWYVDGVEVHNAKTKPRE
EQYNSTYRVVSVLTVLHQDWLNGKEYKCKVSNKALPAPIEKTISK
AKGQPREPQVYTLPPSREEMTKNQVSLWCLVKGFYPSDIAVEWES
NGQPENNYKTTPPVLDSDGSFFLYSKLTVDKSRWQQGNVFSCSVM
HEALHNHYTQKSLSLSPGKGGGGSGGGGSLPETGGHHHHHHHHHH*.
(Ros9a Scaffold 21)
Chain B:
(SEQ ID NO: 646)
MMGVKVLFALICIAVAEAAIQMTQSPSSLSASVGDRVTITCRASQ
DIRNYLNWYQQKPGKAPKLLIYYTSRLESGVPSRFSGSGSGTDYT
LTISSLQPEDFATYYCQQGNTLPWTFGQGTKVEIKGGGGSGGGGS
GGGGSGGGGSGGGSEVQLVESGGGLVQPGGSLRLSCAASGYSFTG
YTMNWVRQAPGKGLEWVALINPYKGVSTYNQKFKDRFTISVDKSK
NTAYLQMNSLRAEDTAVYYCARSGYYGDSDWYFDVWGQGTLVTVS
SGGGGSDAGVTQSPTHLIKTRGQQVTLRCSPKSGHDTVSWYQQAL
GQGPQFIFQYYEEEERQRGNFPDRFSGHQFPNYSSELNVNALLLG
DSALYLCASSLGEGRVDGYTFGSGTRLTVVEDLKNVFPPEVAVFE
PSKAEISRTQKATLVCLATGFYPPHVELSWWVNGKEVHDGVCTDP
QPLKEQPALQDSRYALSSRLRVSATFWQDPRNHFRCQVQFYGLSE
NDEWTQDRAKPVTQIVSAEAWGRADCGGGGSGGGGSGGGGSDKTH
TCPPCPAPELLGGPSVFLFPPKPKDTLEASRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSREEMT
KNQVSLSCAVKGFYPSDIAVEWESNGQPENNYKTTPPVLDSDGSF
FLVSKLTVDKSRWQQGNVFSCSVMHEALHNAYTQKSLSLSPGKGG
GGSGLNDIFEAQKIEWHE.
(Ros9a Scaffold 21)

SEQ ID NOs: 645-646 consists of the following segments from N terminal
to C terminal as shown in Table 8C:
Chain A

| Signal Peptide | MMGVKVLFALICIAVAEAD (SEQ ID NO: 667) | |
|---|---|---|
| TCR-alpha chain | LAKTTQPISVDSYEGQEVNITCSHNNIATNDYITWYQQFPSQGPRF | |
| | IIQGYKTKVTNEVASLFIPADRKSSTLSLPRVSLSDTAVYYCLVGD | |
| | MDQAGTALIFGKGTTLSVSSDYIQNPDPAVYQLRDSKSSDKFVCL | |
| | FTDFDSQIQVSQSKDSDVYITDKCVLDMRSMDFKSNSAVAWSQKS | |
| | DFTCANAFQNSIIPEDTFFPSPESSC (SEQ ID NO: 680) | |
| | TRAV | LAKTTQPISVDSYEGQEVNITCSHNNIATNDYITWYQQFPS |
| | | QGPRFIIQGYKTKVTNEVASLFIPADRKSSTLSLPRVSLSDT |
| | | AVYYCLVGDMDQAGTALIFGKGTTLSVSSD (SEQ ID NO: |
| | | 619) |
| | | CDR1: NIATNDY (SEQ ID NO: 874) |
| | | CDR2: GYKTK (SEQ ID NO: 875) |
| | | CDR3: LVGDMDQAGTALI (SEQ ID NO: 876) |
| | | (IMGT) |
| | TRAC | YIQNPDPAVYQLRDSKSSDKFVCLFTDFDSQIQVSQSKDS |
| | | DVYITDKCVLDMRSMDFKSNSAVAWSQKSDFTCANAFQ |
| | | NSIIPEDTFFPSPESSC (SEQ ID NO: 681) |

| | |
|---|---|
| Linker | GGGGSGGGGSGGGGS (SEQ ID NO: 690) |
| Immunoglobulin | DKTHTCPPCPAPELLGGPSVFLFPPKPKDTLMISRTPEVTCVVVDVS |
| Fc domain chain | HEDPEVKFNWYVDGVEVHNAKTKPREEQYNSTYRVVSVLTVLHQDWL |
| 1 | NGKEYKCKVSNKALPAPIEKTISKAKGQPREPQVYTLPPSREEMTKN |
| | QVSLWCLVKGFYPSDIAVEWESNGQPENNYKTTPPVLDSDGSFFLYS |
| | KLTVDKSRWQQGNVFSCSVMHEALHNHYTQKSLSLSPGK (SEQ ID |
| | NO: 691) |
| Linker with | GGGGSGGGGSLPETGGHHHHHHHHHH (SEQ ID NO: 695) |
| SLPETG and | |
| HIS-tag | |

Chain B

| Signal Peptide | MMGVKVLFALICIAVAEAD (SEQ ID NO: 667) | |
|---|---|---|
| Anti-CD3 VL | AIQMTQSPSSLSASVGDRVTITCRASQDIRNYLNWYQQKPGKAPKLLI | |
| domain | YYTSRLESGVPSRFSGSGSGTDYTLTISSLQPEDFATYYCQQGNTLPW | |
| | TFGQGTKVEIK (SEQ ID NO: 604) | |
| | LC CDR1 QDIRNY (SEQ ID NO: 732), | |
| | LC CDR2 YTS (SEQ ID NO: 733), | |
| | LC CDR3 QQGNTLPWT (SEQ ID NO: 610). | |
| | (IMGT numbering scheme) | |
| linker | GGGGSGGGGSGGGGSGGGGSGGGS (SEQ ID NO: 682) | |
| Anti-CD3 VH | EVQLVESGGGLVQPGGSLRLSCAASGYSFTGYTMNWVRQAPGKGLE | |
| domain | WVALINPYKGVSTYNQKFKDRFTISVDKSKNTAYLQMNSLRAEDTA | |
| | VYYCARSGYYGDSDWYFDVWGQGTLVTVSS (SEQ ID NO: 603) | |
| | HC CDR1 : GYSFTGYT (SEQ ID NO: 729), | |
| | HC CDR2 INPYKGVS (SEQ ID NO: 730), | |
| | HC CDR3 ARSGYYGDSDWYFDV (SEQ ID NO: 731); | |
| | (IMGT numbering scheme) | |
| linker | GGGGSD (SEQ ID NO: 693) | |
| TCR-beta chain | AGVTQSPTHLIKTRGQQVTLRCSPKSGHDTVSWYQQALGQGPQFIFQ | |
| (TRBV:TRBC | YYEEEERQRGNFPDRFSGHQFPNYSSELNVNALLLGDSALYLCASSL | |
| fusion) | GEGRVDGYTFGSGTRLTVVEDLKNVFPPEVAVFEPSKAEISRTQKAT | |
| | LVCLATGFYPPHVELSWWVNGKEVHDGVCTDPQPLKEQPALQDSRYA | |
| | LSSRLRVSATFWQDPRNHFRCQVQFYGLSENDEWTQDRAKPVTQIVS | |
| | AEAWGRADC (SEQ ID NO: 684) | |
| | TRBV | AGVTQSPTHLIKTRGQQVTLRCSPKSGHDTVSWYQQALGQ |
| | | GPQFIFQYYEEEERQRGNFPDRFSGHQFPNYSSELNVNAL |
| | | LLGDSALYLCASSLGEGRVDGYTFGSGTRLTVV |
| | | (SEQ ID NO: 620) |
| | | CDR1: SGHDT (SEQ ID NO: 877); |
| | | CDR2: YYEEEE (SEQ ID NO: 878); |
| | | CDR3: ASSLGEGRVDGYT (SEQ ID NO: 879). |
| | TRBC | EDLKNVFPPEVAVFEPSKAEISRTQKATLVCLATGFYPPHVE |
| | | LSWWVNGKEVHDGVCTDPQPLKEQPALQDSRYALSSRLR |
| | | VSATFWQDPRNHFRCQVQFYGLSENDEWTQDRAKPVTQIV |
| | | SAEAWGRADC (SEQ ID NO: 685) |

| | |
|---|---|
| linker | GGGGSGGGGSGGGGS (SEQ ID NO: 690) |
| Immunoglobulin | DKTHTCPPCPAPELLGGPSVFLFPPKPKDTLEASRTPEVTCVVVDVS |
| Fc domain chain | HEDPEVKFNWYVDGVEVHNAKTKPREEQYNSTYRVVSVLTVLHQDWL |
| 2 | NGKEYKCKVSNKALPAPIEKTISKAKGQPREPQVYTLPPSREEMTKN |
| | QVSLSCAVKGFYPSDIAVEWESNGQPENNYKTTPPVLDSDGSFFLVSK |
| | LTVDKSRWQQGNVFSCSVMHEALHNAYTQKSLSLSPGK (SEQ ID |
| | NO: 692) |
| linker and BAP | GGGGSGLNDIFEAQKIEWHE (SEQ ID NO: 694) |
| tag | |

Ros9d Scaffold 19. The diagrammatic representation of the general design and sequence is depicted in FIG. 10A.

TABLE 8D
MMGVKVLFALICIAVAEADLAKTTQPISVDSYEGQEVNITCSHNNIATNDYITWYQQF
PSQGPRFIIQGYKTKVTNEVASLFIPADRKSSTLSLPRVSLSDTAVYYCLVGDMDQAG
TALIFGKGTTLSVSSDYIQNPDPAVYQLRDSKSSDKFVCLFTDFDSQIQVSQSKDSDVY
ITDKCVLDMRSMDFKSNSAVAWSQKSDFTCANAFQNSIIPEDTFFPSPESSCGSGHHH
HHHHHHHRIRRSGSGVKQTLNFDLLKLAGDVESNPGPMMGVKVLFALICIAVAEAAIQM
TQSPSSLSASVGDRVTITCRASQDIRNYLNWYQQKPGKAPKLLIYYTSRLESGVPSRFSGS
GSGTDYTLTISSLQPEDFATYYCQQGNTLPWTFGQGTKVEIKGGGGSGGGGSGGGGSGG
GGSGGGSEVQLVESGGGLVQPGGSLRLSCAASGYSFTGYTMNWVRQAPGKGLEWVALI
NPYKGVSTYNQKFKDRFTISVDKSKNTAYLQMNSLRAEDTAVYYCARSGYYGDSDWYF
DVWGQGTLVTVSSGGGGSAGVTQSPTHLIKTRGQQVTLRCSPKSGHDTVSWYQQALGQ
GPQFIFQYYEEEERQRGNFPDRFSGHQFPNYSSELNVNALLLGDSALYLCASSLGQTNYG
YTFGSGTRLTVVEDLKNVFPPEVAVFEPSKAEISRTQKATLVCLATGFYPPHVELSWWVN
GKEVHDGVCTDPQPLKEQPALQDSRYALSSRLRVSATFWQDPRNHFRCQVQFYGLSEND
EWTQDRAKPVTQIVSAEAWGRADCGGGGSGLNDIFEAQKIEWHEGGGGSGGGGSLPET
GG* (Ros9d Scaffold 19) (SEQ ID NO: 647).

| Domain | Sequence |
|---|---|
| Signal Peptide | MMGVKVLFALICIAVAEAD (SEQ ID NO: 667) |
| TCR alpha chain | LAKTTQPISVDSYEGQEVNITCSHNNIATNDYITWYQQFPSQGPRFIIQG |
| (TRAV:TRAC | YKTKVTNEVASLFIPADRKSSTLSLPRVSLSDTAVYYCLVGDMDQAGT |
| fusion) | ALIFGKGTTLSVSSDYIQNPDPAVYQLRDSKSSDKFVCLFTDFDSQIQV |
| | SQSKDSDVYITDKCVLDMRSMDFKSNSAVAWSQKSDFTCANAFQNSII |
| | PEDTFFPSPESSC (SEQ ID NO: 680) |
| Linker, HIS-tag, | GSGHHHHHHHHHHRIRRSGSGVKQTLNFDLLKLAGDVESNPGP (SEQ |
| cleavable linker | ID NO: 696) |
| Signa Peptide | MMGVKVLFALICIAVAEA (SEQ ID NO: 668) |
| Anti-CD3-VL | AIQMTQSPSSLSASVGDRVTITCRASQDIRNYLNWYQQKPGKAPKLLI |
| | YYTSRLESGVPSRFSGSGSGTDYTLTISSLQPEDFATYYCQQGNTLPWT |
| | FGQGTKVEIK (SEQ ID NO: 604) |
| | LC CDR1 QDIRNY (SEQ ID NO: 732), |
| | LC CDR2 YTS (SEQ ID NO: 733), |
| | LC CDR3 QQGNTLPWT (SEQ ID NO: 610). |
| | (IMGT numbering scheme) |
| linker | GGGGSGGGGSGGGGSGGGGSGGGS (SEQ ID NO: 682) |
| Anti-CD-VH | EVQLVESGGGLVQPGGSLRLSCAASGYSFTGYTMNWVRQAPGKGLE |
| | WVALINPYKGVSTYNQKFKDRFTISVDKSKNTAYLQMNSLRAEDTAV |
| | YYCARSGYYGDSDWYFDVWGQGTLVTVSS (SEQ ID NO: 603) |
| | HC CDR1 : GYSFTGYT (SEQ ID NO: 729), |
| | HC CDR2 INPYKGVS (SEQ ID NO: 730), |
| | HC CDR3 ARSGYYGDSDWYFDV (SEQ ID NO: 731); |
| | (IMGT numbering scheme) |
| linker | GGGGS (SEQ ID NO: 683) |
| TCR beta chain | AGVTQSPTHLIKTRGQQVTLRCSPKSGHDTVSWYQQALGQGPQFIFQY |
| (TRBV:TRBC | YEEEERQRGNFPDRFSGHQFPNYSSELNVNALLLGDSALYLCASSLGQ |
| fusion) | TNYGYTFGSGTRLTVVEDLKNVFPPEVAVFEPSKAEISRTQKATLVCL |
| | ATGFYPPHVELSWWVNGKEVHDGVCTDPQPLKEQPALQDSRYALSSR |
| | LRVSATFWQDPRNHFRCQVQFYGLSENDEWTQDRAKPVTQIVSAEA |
| | WGRADC (SEQ ID NO: 684) |
| linker-BAP- | GGGGSGLNDIFEAQKIEWHEGGGGSGGGGSLPETGG (SEQ ID NO: |
| LPETG tag | 686) |

The polypeptide Ros9d Scaffold 19 can also be Without signal peptide and having the SEQ ID NO: 648.

Ros9d Scaffold 21: The diagrammatic representation of the general design and sequence is depicted in FIG. 11A. This scaffold has 2 polypeptide chains, the first polypeptide chain (Chain A) has the following sequence

Chain A:
(SEQ ID NO: 649)
MMGVKVLFALICIAVAEADLAKTTQPISVDSYEGQEVNITCSHNN
IATNDYITWYQQFPSQGPRFIIQGYKTKVTNEVASLFIPADRKSS
TLSLPRVSLSDTAVYYCLVGDMDQAGTALIFGKGTTLSVSSDYIQ
NPDPAVYQLRDSKSSDKFVCLFTDFDSQIQVSQSKDSDVYITDKC
VLDMRSMDFKSNSAVAWSQKSDFTCANAFQNSIIPEDTFFPSPES
SCGGGGSGGGGSGGGGSDKTHTCPPCPAPELLGGPSVFLFPPKPK
DTLMISRTPEVTCVVVDVSHEDPEVKFNWYVDGVEVHNAKTKPRE
EQYNSTYRVVSVLTVLHQDWLNGKEYKCKVSNKALPAPIEKTISK
AKGQPREPQVYTLPPSREEMTKNQVSLWCLVKGFYPSDIAVEWES
NGQPENNYKTTPPVLDSDGSFFLYSKLTVDKSRWQQGNVFSCSVM
HEALHNHYTQKSLSLSPGKGGGGSGGGGSLPETGGHHHHHHHHH*.
(Ros9d Scaffold 21)

The second polypeptide (Chain B) has the sequence:

Chain B:
(SEQ ID NO: 650)
MMGVKVLFALICIAVAEAAIQMTQSPSSLSASVGDRVTITCRASQ
DIRNYLNWYQQKPGKAPKLLIYYTSRLESGVPSRFSGSGSGTDYT
LTISSLQPEDFATYYCQQGNTLPWTFGQGTKVEIKGGGGSGGGGS
GGGGSGGGGSGGGSEVQLVESGGGLVQPGGSLRLSCAASGYSFTG
YTMNWVRQAPGKGLEWVALINPYKGVSTYNQKFKDRFTISVDKSK
NTAYLQMNSLRAEDTAVYYCARSGYYGDSDWYFDVWGQGTLVTVS
SGGGGSDAGVTQSPTHLIKTRGQQVTLRCSPKSGHDTVSWYQQAL
GQGPQFIFQYYEEEERQRGNFPDRFSGHQFPNYSSELNVNALLLG
DSALYLCASSLGQTNYGYTFGSGTRLTVVEDLKNVFPPEVAVFEP
SKAEISRTQKATLVCLATGFYPPHVELSWWVNGKEVHDGVCTDPQ
PLKEQPALQDSRYALSSRLRVSATFWQDPRNHFRCQVQFYGLSEN
DEWTQDRAKPVTQIVSAEAWGRADCGGGGSGGGGSGGGGSDKTHT
CPPCPAPELLGGPSVFLFPPKPKDTLEASRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSREEMTK
NQVSLSCAVKGFYPSDIAVEWESNGQPENNYKTTPPVLDSDGSFF
LVSKLTVDKSRWQQGNVFSCSVMHEALHNAYTQKSLSLSPGKGGG
GSGLNDIFEAQKIEWHE*
(Ros9d Scaffold 21).

The above scaffold has the following parts as shown in Table 8E:
Chain A

| | |
|---|---|
| Signal | MMGVKVLFALICIAVAEAD (SEQ ID NO: 667) |
| Peptide | |
| TRAV: | LAKTTQPISVDSYEGQEVNITCSHNNIATNDYITWYQQFPSQGPRFIIQGYKT |
| TRAC | KVTNEVASLFIPADRKSSTLSLPRVSLSDTAVYYCLVGDMDQAGTALIFGKG |
| fusion | TTLSVSSDYIQNPDPAVYQLRDSKSSDKFVCLFTDFDSQIQVSQSKDSDVYIT |
| | DKCVLDMRSMDFKSNSAVAWSQKSDFTCANAFQNSIIPEDTFFPSPESSC |
| | (SEQ ID NO: 681) |
| linker | GGGGSGGGGSGGGGS (SEQ ID NO: 690) |
| Immunoglobulin | DKTHTCPPCPAPELLGGPSVFLFPPKPKDTLMISRTPEVTCVVVDVSHEDPEV |
| Fc | KFNWYVDGVEVHNAKTKPREEQYNSTYRVVSVLTVLHQDWLNGKEYKCK |
| | VSNKALPAPIEKTISKAKGQPREPQVYTLPPSREEMTKNQVSLWCLVKGFYP |
| | SDIAVEWESNGQPENNYKTTPPVLDSDGSFFLYSKLTVDKSRWQQGNVFSC |
| | SVMHEALHNHYTQKSLSLSPGK (SEQ ID NO: 691). |
| linker, | GGGGSGGGGSLPETGGHHHHHHHHHH (SEQ ID NO: 695) |
| LPETG- | |
| HIS Tag | |

Chain B

| | |
|---|---|
| Signal | MMGVKVLFALICIAVAEA (SEQ ID NO: 668) |
| Peptide | |
| Anti-CD3 | AIQMTQSPSSLSASVGDRVTITCRASQDIRNYLNWYQQKPGKAPKLLIYYTS |
| VL | RLESGVPSRFSGSGSGTDYTLTISSLQPEDFATYYCQQGNTLPWTFGQGTKV |
| | EIK (SEQ ID NO: 604) |
| | LC CDR1 QDIRNY (SEQ ID NO: 732), |
| | LC CDR2 YTS (SEQ ID NO: 733), |
| | LC CDR3 QQGNTLPWT (SEQ ID NO: 610). |
| | (IMGT numbering scheme) |
| Linker | GGGGSGGGGSGGGGSGGGGSGGGS (SEQ ID NO: 682) |
| Anti-CD3 | EVQLVESGGGLVQPGGSLRLSCAASGYSFTGYTMNWVRQAPGKGLEWVAL |
| -VH | INPYKGVSTYNQKFKDRFTISVDKSKNTAYLQMNSLRAEDTAVYYCARSGY |
| | YGDSDWYFDVWGQGTLVTVSSGGGGS (SEQ ID NO: 603) |
| | HC CDR1: GYSFTGYT (SEQ ID NO: 729), |
| | HC CDR2 INPYKGVS (SEQ ID NO: 730), |
| | HC CDR3 ARSGYYGDSDWYFDV (SEQ ID NO: 731); |
| | (IMGT numbering scheme) |
| TRBV: | DAGVTQSPTHLIKTRGQQVTLRCSPKSGHDTVSWYQQALGQGPQFIFQYYE |
| TRBC fusion | EEERQRGNFPDRFSGHQFPNYSSELNVNALLLGDSALYLCASSLGQTNYGYT |
| | FGSGTRLTVVEDLKNVFPPEVAVFEPSKAEISRTQKATLVCLATGFYPPHVEL |
| | SWWVNGKEVHDGVCTDPQPLKEQPALQDSRYALSSRLRVSATFWQDPRNH |
| | FRCQVQFYGLSENDEWTQDRAKPVTQIVSAEAWGRADC (SEQ ID NO: 697) |
| Linker | GGGGSGGGGSGGGGS (SEQ ID NO: 690) |
| Immunoglobulin | DKTHTCPPCPAPELLGGPSVFLFPPKPKDTLEASRTPEVTCVVVDVSHEDPEV |
| Fc | KFNWYVDGVEVHNAKTKPREEQYNSTYRVVSVLTVLHQDWLNGKEYKCK |
| region | VSNKALPAPIEKTISKAKGQPREPQVYTLPPSREEMTKNQVSLSCAVKGFYPS |
| | DIAVEWESNGQPENNYKTTPPVLDSDGSFFLVSKLTVDKSRWQQGNVFSCS |
| | VMHEALHNAYTQKSLSLSPGK (SEQ ID NO: 692) |
| Linker and | GGGGSGLNDIFEAQKIEWHE (SEQ ID NO: 694) |
| tag | |

Ros10 Scaffold 19. The diagrammatic representation of the general design and sequence is depicted in FIG. 12A.

The above scaffold has the following parts as shown in Table 8F:
MMGVKVLFALICIAVAEAQKEVEQNSGPLSVPEGAIASLNCTYSDRGSQSFFWYRQYS
GKSPELIMFIYSNGDKEDGRFTAQLNKASQYVSLLIRDSQPSDSATYLCAAAMDSSYK
LIFGSGTRLLVRPDYIQNPDPAVYQLRDSKSSDKFVCLFTDFDSQIQVSQSKDSDVYIT
DKCVLDMRSMDFKSNSAVAWSQKSDFTCANAFQNSIIPEDTFFPSPESSCGSGHHHHH
HHHHHRIRRSGSGVKQTLNFDLLKLAGDVESNPGPMMGVKVLFALICIAVAEAAIQMTQ
SPSSLSASVGDRVTITCRASQDIRNYLNWYQQKPGKAPKLLIYYTSRLESGVPSRFSGSGS
GTDYTLTISSLQPEDFATYYCQQGNTLPWTFGQGTKVEIKGGGGSGGGGSGGGGSGGGG
SGGGSEVQLVESGGGLVQPGGSLRLSCAASGYSFTGYTMNWVRQAPGKGLEWVALINP
YKGVSTYNQKFKDRFTISVDKSKNTAYLQMNSLRAEDTAVYYCARSGYYGDSDWYFDV
WGQGTLVTVSSGGGGSAGITQSPRYKITETGROVTLMCHQTWSHSYMFWYRQDLGHGL
RLIYYSAAADITDKGEVPDGYVVSRSKTENFPLTLESATRSQTSVYFCASSDPGTEAFFGQ
GTRLTVVEDLKNVFPPEVAVFEPSKAEISRTQKATLVCLATGFYPPHVELSWWVNGKEV
HDGVCTDPQPLKEQPALQDSRYALSSRLRVSATFWQDPRNHFRCQVQFYGLSENDEWTQ
DRAKPVTQIVSAEAWGRADCGGGGSGLNDIFEAQKIEWHEGGGGSGGGGSLPETGG
(Ros10 Scaffold 19) (SEQ ID NO: 651).

| Domain | Sequence |
|---|---|
| Signal | MMGVKVLFALICIAVAEA (SEQ ID NO: 668) |
| peptide | |
| TRAV: | QKEVEQNSGPLSVPEGAIASLNCTYSDRGSQSFFWYRQYSGKSPELIMFIYS |
| TRAC | NGDKEDGRFTAQLNKASQYVSLLIRDSQPSDSATYLCAAAMDSSYKLIFGS |
| fusion | GTRLLVRPDYIQNPDPAVYQLRDSKSSDKFVCLFTDFDSQIQVSQSKDSDV |
| | YITDKCVLDMRSMDFKSNSAVAWSQKSDFTCANAFQNSIIPEDTFFPSPESS |
| | C (SEQ ID NO: 698) |
| Linker with | GSGHHHHHHHHHHRIRRSGSGVKQTLNFDLLKLAGDVESNPGP (SEQ ID |
| cleavable | NO: 699) |
| sequence | |
| Signal | MMGVKVLFALICIAVAEA (SEQ ID NO: 668) |
| peptide | |
| Anti-CD3- | AIQMTQSPSSLSASVGDRVTITCRASQDIRNYLNWYQQKPGKAPKLLIYYTS |
| VL | RLESGVPSRFSGSGSGTDYTLTISSLQPEDFATYYCQQGNTLPWTFGQGTKV |
| | EIK (SEQ ID NO: 604) |
| | LC CDR1 QDIRNY (SEQ ID NO: 732), |
| | LC CDR2 YTS (SEQ ID NO: 733), |
| | LC CDR3 QQGNTLPWT (SEQ ID NO: 610); |
| linker | GGGGSGGGGSGGGGSGGGGSGGGS (SEQ ID NO: 682) |
| Anti-CD3 | EVQLVESGGGLVQPGGSLRLSCAASGYSFTGYTMNWVRQAPGKGLEWVA |
| VH | LINPYKGVSTYNQKFKDRFTISVDKSKNTAYLQMNSLRAEDTAVYYCARS |
| | GYYGDSDWYFDVWGQGTLVTVSS (SEQ ID NO: 603) |
| | HC CDR1 : GYSFTGYT (SEQ ID NO: 729), |
| | HC CDR2 INPYKGVS (SEQ ID NO: 730), |
| | HC CDR3 ARSGYYGDSDWYFDV (SEQ ID NO: 731); |
| | (IMGT numbering scheme) |
| Linker | GGGGS (SEQ ID NO: 683) |
| TRBV:TRBC | AGITQSPRYKITETGRQVTLMCHQTWSHSYMFWYRQDLGHGLRLIYYSAA |
| fusion | ADITDKGEVPDGYVVSRSKTENFPLTLESATRSQTSVYFCASSDPGTEAF |
| | FGQGTRLTVVEDLKNVFPPEVAVFEPSKAEISRTQKATLVCLATGFYPPH |
| | VELSWWVNGKEVHDGVCTDPQPLKEQPALQDSRYALSSRLRVSATFWQDP |
| | RNHFRCQVQFYGLSENDEWTQDRAKPVTQIVSAEAWGRADC |
| | (SEQ ID NO: 700) |
| linker- | GGGGSGLNDIFEAQKIEWHEGGGGSGGGGSLPETGG (SEQ ID NO: 686) |
| BAP- | |
| LPETG tag | |

### Example 3. Generation and Purification of sTCR Scaffolds

In this example, sTCRs are isolated in small scale productions for testing the functionality. Human epithelial cells Expi293 were transfected with mRNA constructs (or alternatively with plasmid vectors consisting of the sequence encoding an sTCR, Cells were grown in culture for a week and the supernatants collected. The secreted sTCR products were passed through IMAC nickel NTA columns and size exclusion gel filtration columns and collected. Typically, the process yields an estimated 5-30 mg/L of culture supernatant. Shown in FIG. 4 (top), a cartoon of a cell culture from left collection of the purified product. FIG. 4 bottom shows representative purification products run on gel electrophoresis. FIGS. 5A and 5B additionally show purification products of scaffold 2 eluents collected at different times, run on a native gel: left early collection, right, mid-elution. Right-most, protein size marker. FIG. 5B shows single elution peak.

### Example 4. Functional Assay of sTCR Scaffolds on Cells Transfected with Peptide and HLA

In this example, functional assays of the bispecific TCRs were performed. FIG. 6 shows a schematic diagram of the process. The effector cells are T cells from a human donor. Target cells are A375 cells expressing the HLA and the epitope specific for each sTCR. A375 cells were electroporated with vector or mRNA expressing the HLA, and the KRAS mutated peptide and rested overnight. Effector T cells were purified from peripheral blood sample of a healthy donor and rested for 1-2 days in non-activating media before assay. sTCRs were added to the media and the Effector to target cell rations were between 0.04:1-10:1. Survival of the A375 cells were evaluated. FIGS. 7A-12B illustrate the results for four representative bispecific sTCR scaffolds consisting of Ros9 and Ros 10 TCR backbones having TRAV and TRBV sequences against RAS G12D peptide GADGVGKSA (KRAS G12D 9mer) on HLA-C*08:02; and an anti CD3 scFv. The amino acid sequences and representative diagrammatic view of the structures are provided for each set of experiment with a different bispecific molecule on FIGS. 7A, 8A, 9A, 10A, 11A, and 12A. The amino acid sequences of the are color coded in accordance to the fill color scheme of the domains in the corresponding diagram of the BiTE scaffold structure to the left. The corresponding results for cytotoxic assay is represented in the graphs on the FIGS. 7B, 8B, 9B, 10B, 11B and 12B respectively. The higher the cytotoxicity, in this case, the lower the values on the Y axis, determining the total green object area, since the target cells express green fluorescence protein (GFP). FIG. 7B shows, Scaffold 19 shows higher cytotoxicity of the target cells with increasing sTCR concentration. This indicated that the sTCR Scaffold 19 potentiated T cell activation and cytotoxicity. Likewise, FIG. 9B showed similar effect with Scaffold 21; FIG. 10B with Scaffold 19, and FIG. 11B with Scaffold 21. However, data shown in FIG. 8B and FIG. 12B indicate that the scaffolds shown in these figures did not result in successful potentiation of the T cell in inducing cytotoxicity to target cells.

### Example 5. Functional Assay of sTCR Scaffolds on Tumor Cells Expressing Endogenous Peptide and HLA

In this example, effectiveness of the representative sTCR scaffolds are evaluated with tumor cells as target cells that express the peptide and HLA for the TCR. HPAC cells represent tumor cell line that are not engineered and known to express the KRAS G12D mutation, and HLA-C*08:02. One goal of this experiment is to determine if tumor cell lines with native levels of HLA/neoantigen have sufficient antigen to be targeted by BiTE, and also to compare BiTE efficacy across scaffolds (CD3 binders; affinity) against physiologically relevant tumor cells. Cells were stained for presence of Annexin V and Caspase 3/7 (nuc-light rapid in future tests) as apoptotic cell markers after the treatment—as depicted in a representative schematic diagram in FIG. 13.

The Ros 9 Scaffold 19 and Ros 10 Scaffold 19 structures were compared in this assay and results presented in data on FIGS. 14A-14C. FIG. 14A shows fluorescent images of target cells positive for Annexin V (orange) and caspase 3/7 apoptotic signals (green), with the indicated doses of sTCRs in culture and T isolated cells from a human donor. As in indicated, presence of T cells and the sTCR at the highest concentration of 100 microM as well as at 50 microM showed high expression of Annexin V at least in case of incubation with Scaffold 19 (top panel). It was noted from this assay that the caspase signal was detectable throughout and including in the control sets, particularly, one without any T cells, second from left, indicating it was not a clear signal. The quantified data from this assay is represented in FIG. 14B. Both FIGS. 14A and 14B indicate that Ros 9a Scaffold 19 showed efficient target cell killing at the highest doses of the BiTE sTCRs. Ros 10 Scaffold 19 in showed modest cell cytotoxicity in a dose dependent manner, however even at the highest doses, did not yield robust cell killing such as Ros9a Scaffold 19 BiTEs, which is in keeping with the data found in the previous assay with transfected target cells, depicted in FIG. 12B. FIG. 14C shows a repetition of the assay with Annexin V staining alone. The results are in keeping with the data shown in FIG. 14B.

In another reiteration of the assay, T cells were rested for shorter period, keeping all other conditions the same. Results are shown in FIGS. 15A and 15B. The fluorescent images shown in FIG. 15A and the quantitative results in FIG. 15B indicate a higher cell cytotoxicity at much lower doses of the sTCR, which may indicate that the T cells are losing their cytotoxic potential and responsiveness with longer incubation in culture media, and becoming anergic.

### Example 6. Bispecific sTCR with Different CD3 scFv on Scaffold 22

In an exemplary design, an anti-CD3 binder domain is an scFv having the sequence

(SEQ ID NO: 701)
QVQLVQSGGGLVQPGGPLRLSCTASGFSFSNNYYMCWVRQAPGKG
LEWVSCIYINNIGSTWYASWAKGRFTISRDNSKNTLYLQMNSLRA
EDTAVYYCARGIYSDSSDFIGNLWGQGTLVTVSSGGGGSGGGGSG
GGGSGGGGSAIQMTQSPSSLSASVGDRVTITCQASQTIYNYLAWY
QQKPGKVPELLIYKASTLASGVPSRFMGTGFGTDFTLTIDSLQPE
DAATYYCQQYNTIINVDRTFGGGTKVEIK.

HC CDRs and L C CDRs underlined according to IMGT scheme respectively.

In another exemplary design, an anti-CD3 binder domain is an scFv comprising the amino acid sequence:

(SEQ ID NO: 702)
AIQMTQSPSSLSASVGDRVTITCQASQTIYNYLAWYQQKPGKVPE
LLIYKASTLASGVPSRFMGTGFGTDFTLTIDSLQPEDAATYYCQQ
YNTIINVDRTFGGGTKVEIKGGGGSGGGGSGGGGSGGGGSGGGGS
QVQLVQSGGGLVQPGGPLRLSCTASGFSFSNNYYMCWVRQAPGKG
LEWVSCIYTNNIGSTWYASWAKGRFTISRDNSKNTLYLQMNSLRA
EDTAVYYCARGIYSDSSDFIGNLWGQGTLVTVSS

LC CDRs and HC CDRs underlined according to IMGT scheme respectively.

The following example shows design and construction of bispecific sTCRs Table. 9. In this table the HCDR3 are designated in bold and italics. HCDR1 and 2 are designated as underlined. LCDRs are designated in bold.

TABLE 9
Constructs on Scaffold 22.

| Construct | | | SEQ ID |
|---|---|---|---|
| name | Amino Acid Sequence | | NO |
| scFv from | MMGVKVLFALICIAVAEADLAKTTQPISVDSYEGQEVNITCSHNNI | | 755 |
| BNT New | ATNDYITWYQQFPSQGPRFIIQGYKTKVTNEVASLFIPADRKSST | | |
| Scaffolds | LSLPRVSLSDTAVYYCLVGDMDQAGTALIFGKGTTLSVSSDYIQN | | |
| group | PDPAVYQLRDSKSSDKFVCLFTDFDSQIQVSQSKDSDVYITDKCV | | |
| (NSG23)- | LDMRSMDFKSNSAVAWSQKSDFTCANAFQNSIIPEDTFFPSPESS | | |
| TR66-VHVL | CGSGHHHHHHHHHHRIRRSGSGVKQTLNFDLLKLAGDVESNPGPM | | |
| Ros9a | MGVKVLFALICIAVAEAQVQLQQSGAELARPGASVKMSCKTSGYTF | | |
| Scaffold 22 | TRYTMHWVKQRPGQGLEWIGYINPSRGYTNYNQKFKDKATLTTDK | | |
| | SSSTAYMQLSSLTSEDSAVYYCARYYDDHYSLDYWGQGTTLTVSSG | | |
| | GGGSGGGGSGGGGSGGGGSQIVLTQSPAIMSASPGEKVTMTCRASS | | |
| | SVSYMNWYQQKSGTSPKRWIYDTSKVASGVPYRFSGSGSGTSYSLT | | |
| | ISSMEAEDAATYYCQQWSSNPLTFGAGTKLELKGGGGSAGVTQSPT | | |
| | HLIKTRGQQVTLRCSPKSGHDTVSWYQQALGOGPQFIFQYYEEEER | | |
| | QRGNFPDRFSGHQFPNYSSELNVNALLLGDSALYLCASSLGEGRVD | | |
| | GYTFGSGTRLTVVEDLKNVFPPEVAVFEPSKAEISRTQKATLVCLAT | | |
| | GFYPPHVELSWWVNGKEVHDGVCTDPQPLKEQPALQDSRYALSSR | | |
| | LRVSATFWQDPRNHFRCQVQFYGLSENDEWTQDRAKPVTQIVSAE | | |
| | AWGRADCGGGGSGLNDIFEAQKIEWHEGGGGSGGGGSLPETGG | | |
| | Signal | MMGVKVLFALICIAVAEAD | 756 |
| | Peptide | | |
| | Human | LAKTTQPISVDSYEGQEVNITCSHNNIATNDYITWYQQF | 757 |
| | TCR-alpha | PSQGPRFIIQGYKTKVTNEVASLFIPADRKSSTLSLPRVS | |
| | chain | LSDTAVYYCLVGDMDQAGTALIFGKGTTLSVSSDYIQN | |
| | | PDPAVYQLRDSKSSDKFVCLFTDFDSQIQVSQSKDSDV | |
| | | YITDKCVLDMRSMDFKSNSAVAWSQKSDFTCANAFQN | |
| | | SIIPEDTFFPSPESSC | |
| | Linker- | GSGHHHHHHHHHHRIRRSGSGVKQTLNFDLLKLAGDV | 758 |
| | 10X HIS | ESNPGP | |
| | (underlined) | | |
| | and | | |
| | cleavage | | |
| | domain | | |
| | (italicized) | | |
| | Second | MMGVKVLFALICIAVAEA | 668 |
| | signal | | |
| | peptide | | |
| | CD3-VH | QVQLQQSGAELARPGASVKMSCKTSGYTFTRYTMHW | 759 |
| | domain | VKQRPGQGLEWIGYINPSRGYTNYNQKFKDKATLTTD | |
| | | KSSSTAYMQLSSLTSEDSAVYYCARYYDDHYSLDYWG | |
| | | QGTTLTVSS | |
| | | (Underlined CDR sequences, Kabat) | |
| | | According to IMGT numbering scheme: | |
| | | HCDR1: GYTFTRYT (SEQ ID NO: 718) | |
| | | HCDR2: INPSRGYT (SEQ ID NO: 719) | |
| | | HCDR3: ARYYDDHYSLDY (SEQ ID NO: 721) | |
| | linker | GGGGSGGGGSGGGGSGGGGS | 760 |
| | CD3-VL | QIVLTQSPAIMSASPGEKVTMTCRASSSVSYMNWYQQK | 761 |
| | domain | SGTSPKRWIYDTSKVASGVPYRFSGSGSGTSYSLTISS | |
| | | MEAEDAATYYCQQWSSNPLTFGAGTKLELK | |
| | | (Underlined CDR sequences, Kabat) | |
| | | According to IMGT numbering scheme: | |
| | | HCDR1: SSVSY (SEQ ID NO: 722) | |
| | | HCDR2: INPSRGYT (SEQ ID NO: 723) | |
| | | HCDR3: ARYYDDHYSLDY (SEQ ID NO: 724) | |
| | linker | GGGGS | 683 |
| | Human | AGVTQSPTHLIKTRGQQVTLRCSPKSGHDTVSWYQQAL | 762 |
| | TCR-beta | GQGPQFIFQYYEEEERQRGNFPDRFSGHQFPNYSSELNV | |
| | TRBV: | NALLLGDSALYLCASSLGEGRVDGYTFGSGTRLTVVED | |
| | TRBC | LKNVFPPEVAVFEPSKAEISRTQKATLVCLATGFYPPHV | |
| | | ELSWWVNGKEVHDGVCTDPQPLKEQPALQDSRYALSS | |
| | | RLRVSATFWQDPRNHFRCQVQFYGLSENDEWTQDRAK | |
| | | PVTQIVSAEAWGRADC | |
| | Linker | GGGGSGLNDIFEAQKIEWHEGGGGSGGGGSLPETGG | 686 |
| | with | | |
| | LPETG | | |
| | sequence | | |
| scFv from | MMGVKVLFALICIAVAEADLAKTTQPISVDSYEGQEVNITCSHNNI | | 763 |
| BNT New | ATNDYITWYQQFPSQGPRFIIQGYKTKVTNEVASLFIPADRKSST | | |
| Scaffolds | LSLPRVSLSDTAVYYCLVGDMDQAGTALIFGKGTTLSVSSDYIQN | | |
| group- | PDPAVYQLRDSKSSDKFVCLFTDFDSQIQVSQSKDSDVYITDKCV | | |
| TR66-VLVH | LDMRSMDFKSNSAVAWSQKSDFTCANAFQNSIIPEDTFFPSPESS | | |
| Ros9a | CGSGHHHHHHHHHHRIRRSGSGVKQTLNFDLLKLAGDVESNPGPM | | |
| Scaffold 22 | MGVKVLFALICIAVAEAQIVLTQSPAIMSASPGEKVTMTCRASSSVS | | |
| | YMNWYQQKSGTSPKRWIYDTSKVASGVPYRFSGSGSGTSYSLTISS | | |
| | MEAEDAATYYCQQWSSNPLTFGAGTKLELKGGGGSGGGGSGGGGS | | |
| | GGGGSGGGGSQVQLQQSGAELARPGASVKMSCKTSGYTFTRYTMH | | |
| | WVKQRPGQGLEWIGYINPSRGYTNYNQKFKDKATLTTDKSSSTAY | | |
| | MQLSSLTSEDSAVYYCARYYDDHYSLDYWGQGTTLTVSSGGGGSA | | |
| | GVTQSPTHLIKTRGQQVTLRCSPKSGHDTVSWYQQALGQGPQFIFQ | | |
| | YYEEEERQRGNFPDRFSGHQFPNYSSELNVNALLLGDSALYLCASSL | | |
| | GEGRVDGYTFGSGTRLTVVEDLKNVFPPEVAVFEPSKAEISRTQKAT | | |
| | LVCLATGFYPPHVELSWWVNGKEVHDGVCTDPQPLKEQPALQDSR | | |
| | YALSSRLRVSATFWQDPRNHFRCQVQFYGLSENDEWTQDRAKPVT | | |
| | QIVSAEAWGRADCGGGGSGLNDIFEAQKIEWHEGGGGSGGGGSLPE | | |
| | TGG* | | |
| | Signal | MMGVKVLFALICIAVAEAD | 756 |
| | peptide | | |
| | Human | LAKTTQPISVDSYEGQEVNITCSHNNIATNDYITWYQQF | 764 |
| | | PSQGPRFIIQGYKTKVTNEVASLFIPADRKSSTLSLPRVS | |
| | | LSDTAVYYCLVGDMDQAGTALIFGKGTTLSVSSDYIQN | |
| | TCR-alpha | PDPAVYQLRDSKSSDKFVCLFTDFDSQIQVSQSKDSDV | |
| | chain | YITDKCVLDMRSMDFKSNSAVAWSQKSDFTCANAFQN | |
| | TRAV: | SIIPEDTFFPSPESSC | |
| | TRAC | | |
| | Linker | GSGHHHHHHHHHHRIRRSGSGVKQTLNFDLLKLAGDV | 765 |
| | 10X HIS | ESNPGP | |
| | and | | |
| | cleavage | | |
| | domain | | |
| | Second | MMGVKVLFALICIAVAEA | 668 |
| | signal | | |
| | peptide | | |
| | Anti-CD3- | QVQLQQSGAELARPGASVKMSCKTSGYTFTRYTMHW | 767 |
| | VH | VKQRPGQGLEWIGYINPSRGYTNYNQKFKDKATLTTD | |
| | domain | KSSSTAYMQLSSLTSEDSAVYYCARYYDDHYSLDYWG | |
| | | QGTTLTVSS | |
| | linker | GGGGSGGGGSGGGGSGGGGSGGGGS | 760 |
| | Anti-CD3- | QIVLTQSPAIMSASPGEKVTMTCRASSSVSYMNWYQQK | 766 |
| | VL domain | SGTSPKRWIYDTSKVASGVPYRFSGSGSGTSYSLTISSM | |
| | | EAEDAATYYCQQWSSNPLTFGAGTKLELK | |
| | Linker | GGGGS | 683 |
| | Human | AGVTQSPTHLIKTRGQQVTLRCSPKSGHDTVSWYQQAL | 768 |
| | TCR-beta | GQGPQFIFQYYEEEERQRGNFPDRFSGHQFPNYSSELNV | |
| | chain: | NALLLGDSALYLCASSLGEGRVDGYTFGSGTRLTVVED | |
| | TRBV: | LKNVFPPEVAVFEPSKAEISRTQKATLVCLATGFYPPHV | |
| | TRBC | ELSWWVNGKEVHDGVCTDPQPLKEQPALQDSRYALSS | |
| | | RLRVSATFWQDPRNHFRCQVQFYGLSENDEWTQDRAK | |
| | | PVTQIVSAEAWGRADC | |
| | linker- | GGGGSGLNDIFEAQKIEWHEGGGGSGGGGSLPETGG | 686 |
| | BAP- | | |
| | LPETG tag | | |
| scFv from | MMGVKVLFALICIAVAEADLAKTTQPISVDSYEGQEVNITCSHNNI | | 769 |
| BNT New | ATNDYITWYQQFPSQGPRFIIQGYKTKVTNEVASLFIPADRKSST | | |
| Scaffolds | LSLPRVSLSDTAVYYCLVGDMDQAGTALIFGKGTTLSVSSDYIQN | | |
| group- | PDPAVYQLRDSKSSDKFVCLFTDFDSQIQVSQSKDSDVYITDKCV | | |
| NSG23- | LDMRSMDFKSNSAVAWSQKSDFTCANAFQNSIIPEDTFFPSPESS | | |
| 210112- | CGSGHHHHHHHHHHRIRRSGSGVKQTLNFDLLKLAGDVESNPGPM | | |
| VHVL | MGVKVLFALICIAVAEAQVQLVQSGGGLVQPGGPLRLSCTASGFSFS | | |
| Ros9a | NNYYMCWVRQAPGKGLEWVSCIYINNIGSTWYASWAKGRFTISRD | | |
| Scaffold 22 | NSKNTLYLQMNSLRAEDTAVYYCARGIYSDSSDFIGNLWGQGTLVT | | |
| | VSSGGGGSGGGGSGGGGSGGGGSAIQMTQSPSSLSASVGDRVTITC | | |
| | QASQTIYNYLAWYQQKPGKVPELLIYKASTLASGVPSRFMGTGFGT | | |
| | DFTLTIDSLQPEDAATYYCQQYNTIINVDRTFGGGTKVEIKGGGGSA | | |
| | GVTQSPTHLIKTRGQQVTLRCSPKSGHDTVSWYQQALGOGPQFIFQ | | |
| | YYEEEERQRGNFPDRFSGHQFPNYSSELNVNALLLGDSALYLCASSL | | |
| | GEGRVDGYTFGSGTRLTVVEDLKNVFPPEVAVFEPSKAEISRTQKAT | | |
| | LVCLATGFYPPHVELSWWVNGKEVHDGVCTDPQPLKEQPALQDSR | | |
| | YALSSRLRVSATFWQDPRNHFRCQVQFYGLSENDEWTQDRAKPVT | | |
| | QIVSAEAWGRADCGGGGSGLNDIFEAQKIEWHEGGGGSGGGGSLPE | | |
| | TGG* | | |
| | Signal | MMGVKVLFALICIAVAEAD | 756 |
| | peptide | | |
| | Human | LAKTTQPISVDSYEGQEVNITCSHNNIATNDYITWYQQF | 770 |
| | TCR-alpha | PSQGPRFIIQGYKTKVTNEVASLFIPADRKSSTLSLPRVS | |
| | chain | LSDTAVYYCLVGDMDQAGTALIFGKGTTLSVSSDYIQN | |
| | | PDPAVYQLRDSKSSDKFVCLFTDFDSQIQVSQSKDSDV | |
| | | YITDKCVLDMRSMDFKSNSAVAWSQKSDFTCANAFQN | |
| | | SIIPEDTFFPSPESSC | |
| | Linker | GSGHHHHHHHHHHRIRRSGSGVKQTLNFDLLKLAGDV | 771 |
| | | ESNPGP | |
| | Second | MMGVKVLFALICIAVAEA | 668 |
| | signal | | |
| | peptide | | |
| | Anti-CD3- | QVQLVQSGGGLVQPGGPLRLSCTASGFSFSNNYYMCW | 772 |
| | VH | VRQAPGKGLEWVSCIYINNIGSTWYASWAKGRFTISRD | |
| | domain | NSKNTLYLQMNSLRAEDTAVYYCARGIYSDSSDFIGNL | |
| | | WGQGTLVTVSS | |
| | | CDRs according to IMGT: | |
| | | GFSFSNNYY (SEQ ID NO: 711) | |
| | | IYTNNIGST (SEQ ID NO: 712) | |
| | | ARGIYSDSSDFIGNL (SEQ ID NO: 713) | |
| | Linker | GGGGSGGGGSGGGGSGGGGS | 760 |
| | Anti-CD3- | AIQMTQSPSSLSASVGDRVTITCQASQTIYNYLAWYQQ | 773 |
| | VL domain | KPGKVPELLIYKASTLASGVPSRFMGTGFGTDFTLTIDS | |
| | | LQPEDAATYYCQQYNTIINVDRTFGGGTKVEIK | |
| | | CDRs underlined according to IMGT: | |
| | | QTIYNY (SEQ ID NO: 714) | |
| | | KAS (SEQ ID NO: 715) | |
| | | QQYNTIINVDRT (SEQ ID NO: 716) | |
| | Linker | GGGGS | 683 |
| | TCR-beta | AGVTQSPTHLIKTRGQQVTLRCSPKSGHDTVSWYQQAL | 774 |
| | chain | GQGPQFIFQYYEEEERQRGNFPDRFSGHQFPNYSSELNV | |
| | | NALLLGDSALYLCASSLGEGRVDGYTFGSGTRLTVVED | |
| | | LKNVFPPEVAVFEPSKAEISRTQKATLVCLATGFYPPHV | |
| | | ELSWWVNGKEVHDGVCTDPQPLKEQPALQDSRYALSS | |
| | | RLRVSATFWQDPRNHFRCQVQFYGLSENDEWTQDRAK | |
| | | PVTQIVSAEAWGRADC | |
| | linker- | GGGGSGLNDIFEAQKIEWHEGGGGSGGGGSLPETGG | 686 |
| | BAP- | | |
| | LPETG tag | | |
| scFv from | YIQNPDPAVYQLRDSKSSDKFVCLFTDFDSQIQVSQSKDSDVYITD | | 775 |
| New | KCVLDMRSMDFKSNSAVAWSQKSDFTCANAFQNSIIPEDTFFPSP | | |
| Scaffolds | ESSCEDLKNVFPPEVAVFEPSKAEISRTQKATLVCLATGFYPPHVELS | | |
| group- | WWVNGKEVHDGVCTDPQPLKEQPALQDSRYALSSRLRVSATFWQD | | |
| NSG23- | PRNHFRCQVQFYGLSENDEWTQDRAKPVTQIVSAEAWGRADCMM | | |
| 210112- | GVKVLFALICIAVAEADLAKTTQPISVDSYEGQEVNITCSHNNIAT | | |
| VLVH | NDYITWYQQFPSQGPRFIIQGYKTKVTNEVASLFIPADRKSSTLSL | | |
| Ros9a | PRVSLSDTAVYYCLVGDMDQAGTALIFGKGTTLSVSSDYIQNPDP | | |
| Scaffold 22 | AVYQLRDSKSSDKFVCLFTDFDSQIQVSQSKDSDVYITDKCVLDM | | |
| | RSMDFKSNSAVAWSQKSDFTCANAFQNSIIPEDTFFPSPESSCGSG | | |
| | HHHHHHHHHHRIRRSGSGVKQTLNFDLLKLAGDVESNPGPMMGVK | | |
| | VLFALICIAVAEAAIQMTQSPSSLSASVGDRVTITCQASQTIYNYLAW | | |
| | YQQKPGKVPELLIYKASTLASGVPSRFMGTGFGTDFTLTIDSLQPED | | |
| | AATYYCQQYNTIINVDRTFGGGTKVEIKGGGGSGGGGSGGGGSGGG | | |
| | GSGGGGSQVQLVQSGGGLVQPGGPLRLSCTASGFSFSNNYYMCWV | | |
| | RQAPGKGLEWVSCIYTNNIGSTWYASWAKGRFTISRDNSKNTLYLQ | | |
| | MNSLRAEDTAVYYCARGIYSDSSDFIGNLWGQGTLVTVSSGGGGSA | | |
| | GVTQSPTHLIKTRGQQVTLRCSPKSGHDTVSWYQQALGOGPQFIFQ | | |
| | YYEEEERQRGNFPDRFSGHQFPNYSSELNVNALLLGDSALYLCASSL | | |
| | GEGRVDGYTFGSGTRLTVVEDLKNVFPPEVAVFEPSKAEISRTQKAT | | |
| | LVCLATGFYPPHVELSWWVNGKEVHDGVCTDPQPLKEQPALQDSR | | |
| | YALSSRLRVSATFWQDPRNHFRCQVQFYGLSENDEWTQDRAKPVT | | |
| | QIVSAEAWGRADCGGGGSGLNDIFEAQKIEWHEGGGGSGGGGSLPE | | |
| | TGG | | |
| | Signal peptide | MMGVKVLFALICIAVAEAD | 756 |
| | sequence | | |
| | Human TCR- | LAKTTQPISVDSYEGQEVNITCSHNNIATNDYITWYQ | 776 |
| | alpha chain | QFPSQGPRFIIQGYKTKVTNEVASLFIPADRKSSTLSL | |
| | | PRVSLSDTAVYYCLVGDMDQAGTALIFGKGTTLSVS | |
| | | SDYIQNPDPAVYQLRDSKSSDKFVCLFTDFDSQIQVS | |
| | | QSKDSDVYITDKCVLDMRSMDFKSNSAVAWSQKSD | |
| | | FTCANAFQNSIIPEDTFFPSPESSC | |
| | His tag | GSGHHHHHHHHHHRIRRSGSGVKQTLNFDLLKLAG | |
| | +Linker | DVESNPGP | |
| | Second signal | MMGVKVLFALICIAVAEA | 668 |
| | peptide | | |
| | Anti-CD3-VL | AIQMTQSPSSLSASVGDRVTITCQASQTIYNYLAWYQ | 778 |
| | domain | QKPGKVPELLIYKASTLASGVPSRFMGTGFGTDFTLT | |
| | [210112-VL] | IDSLQPEDAATYYCQQYNTIINVDRTFGGGTKVEIK | |
| | | (CDRs underlined according to IMGT) | |
| | Linker | GGGGSGGGGSGGGGSGGGGSGGGGS | 760 |
| | Anti-CD3- | QVQLVQSGGGLVQPGGPLRLSCTASGFSFSNNYYMC | 779 |
| | VH domain | WVRQAPGKGLEWVSCIYTNNIGSTWYASWAKGRFT | |
| | [210112-VH] | ISRDNSKNTLYLQMNSLRAEDTAVYYCARGIYSDSS | |
| | | DFIGNLWGQGTLVTVSS | |
| | | (CDRs underlined according to IMGT) | |
| | Linker | GGGGS | 683 |
| | Human TCR- | AGVTQSPTHLIKTRGQQVTLRCSPKSGHDTVSWYQQ | 780 |
| | beta chain | ALGQGPQFIFQYYEEEERQRGNFPDRFSGHQFPNYSS | |
| | | ELNVNALLLGDSALYLCASSLGEGRVDGYTFGSGTR | |
| | | LTVVEDLKNVFPPEVAVFEPSKAEISRTQKATLVCLA | |
| | | TGFYPPHVELSWWVNGKEVHDGVCTDPQPLKEQPA | |
| | | LQDSRYALSSRLRVSATFWQDPRNHFRCQVQFYGLS | |
| | | ENDEWTQDRAKPVTQIVSAEAWGRADC | |
| | linker-BAP- | GGGGSGLNDIFEAQKIEWHEGGGGSGGGGSLPETGG | 686 |
| | LPETG tag | | |
| scFv from | MMGVKVLFALICIAVAEADLAKTTQPISVDSYEGQEVNITCSHNNI | | 781 |
| BNT New | ATNDYITWYQQFPSQGPRFIIQGYKTKVTNEVASLFIPADRKSST | | |
| Scaffolds | LSLPRVSLSDTAVYYCLVGDMDQAGTALIFGKGTTLSVSSDYIQN | | |
| group- | PDPAVYQLRDSKSSDKFVCLFTDFDSQIQVSQSKDSDVYITDKCV | | |
| NSG23- | LDMRSMDFKSNSAVAWSQKSDFTCANAFQNSIIPEDTFFPSPESS | | |
| TR66-VHVL | CGSGHHHHHHHHHHRIRRSGSGVKQTLNFDLLKLAGDVESNPGPM | | |
| Ros9d | MGVKVLFALICIAVAEAQVQLQQSGAELARPGASVKMSCKTSGYTF | | |
| Scaffold 22 | TRYTMHWVKQRPGQGLEWIGYINPSRGYTNYNQKFKDKATLTTDK | | |
| | SSSTAYMQLSSLTSEDSAVYYCARYYDDHYSLDYWGQGTTLTVSSG | | |
| | GGGSGGGGGGGGSGGGGSQIVLTQSPAIMSASPGEKVTMTCRASS | | |
| | SVSYMNWYQQKSGTSPKRWIYDTSKVASGVPYRFSGSGSGTSYSLT | | |
| | ISSMEAEDAATYYCQQWSSNPLTFGAGTKLELKGGGGSAGVTQSPT | | |
| | HLIKTRGQQVTLRCSPKSGHDTVSWYQQALGQGPQFIFQYYEEEER | | |
| | QRGNFPDRFSGHQFPNYSSELNVNALLLGDSALYLCASSLGQTNYG | | |
| | YTFGSGTRLTVVEDLKNVFPPEVAVFEPSKAEISRTQKATLVCLATG | | |
| | FYPPHVELSWWVNGKEVHDGVCTDPQPLKEQPALQDSRYALSSRL | | |
| | RVSATFWQDPRNHFRCQVQFYGLSENDEWTQDRAKPVTQIVSAEA | | |
| | WGRADCGGGGSGLNDIFEAQKIEWHEGGGGSGGGGSLPETGG | | |
| | Signal | MMGVKVLFALICIAVAEAD | 756 |
| | peptide | | |
| | Human | LAKTTQPISVDSYEGQEVNITCSHNNIATNDYITWYQQF | 782 |
| | TCR-alpha | PSQGPRFIIQGYKTKVTNEVASLFIPADRKSSTLSLPRVS | |
| | chain | LSDTAVYYCLVGDMDQAGTALIFGKGTTLSVSSDYIQN | |
| | | PDPAVYQLRDSKSSDKFVCLFTDFDSQIQVSQSKDSDV | |
| | | YITDKCVLDMRSMDFKSNSAVAWSQKSDFTCANAFQN | |
| | | SIIPEDTFFPSPESSC | |
| | Linker | GSGHHHHHHHHHHRIRRSGSGVKQTLNFDLLKLAGDV | |
| | peptide | ESNPGP | |
| | Second | MMGVKVLFALICIAVAEA | 668 |
| | signal | | |
| | peptide | | |
| | sequence | | |
| | Anti-CD3- | QVQLQQSGAELARPGASVKMSCKTSGYTFTRYTMHW | 783 |
| | VH | VKQRPGQGLEWIGYINPSRGYTNYNQKFKDKATLTTD | |
| | domain | KSSSTAYMQLSSLTSEDSAVYYCARYYDDHYSLDYWG | |
| | | QGTTLTVSS | |
| | Linker | GGGGSGGGGSGGGGSGGGGS | 760 |
| | Anti-CD3- | QIVLTQSPAIMSASPGEKVTMTCRASSSVSYMNWYQQK | 784 |
| | VL domain | SGTSPKRWIYDTSKVASGVPYRFSGSGSGTSYSLTISSM | |
| | | EAEDAATYYCQQWSSNPLTFGAGTKLELK | |
| | Linker | GGGGS | 683 |
| | TCR beta | AGVTQSPTHLIKTRGQQVTLRCSPKSGHDTVSWYQQAL | 785 |
| | chain | GQGPQFIFQYYEEEERQRGNFPDRFSGHQFPNYSSELNV | |
| | | NALLLGDSALYLCASSLGQTNYGYTFGSGTRLTVVEDL | |
| | | KNVFPPEVAVFEPSKAEISRTQKATLVCLATGFYPPHVE | |
| | | LSWWVNGKEVHDGVCTDPQPLKEQPALQDSRYALSSR | |
| | | LRVSATFWQDPRNHFRCQVQFYGLSENDEWTQDRAKP | |
| | | VTQIVSAEAWGRADC | |
| | linker- | GGGGSGLNDIFEAQKIEWHEGGGGSGGGGSLPETGG | |
| | BAP- | | |
| | LPETG tag | | |
| scFv from | MMGVKVLFALICIAVAEADLAKTTQPISVDSYEGQEVNITCSHNNI | | 786 |
| BNT New | ATNDYITWYQQFPSQGPRFIIQGYKTKVTNEVASLFIPADRKSST | | |
| Scaffolds | LSLPRVSLSDTAVYYCLVGDMDQAGTALIFGKGTTLSVSSDYIQN | | |
| group- | PDPAVYQLRDSKSSDKFVCLFTDFDSQIQVSQSKDSDVYITDKCV | | |
| NSG23- | LDMRSMDFKSNSAVAWSQKSDFTCANAFQNSIIPEDTFFPSPESS | | |
| 210112- | CGSGHHHHHHHHHHRIRRSGSGVKQTLNFDLLKLAGDVESNPGPM | | |
| VHVL | MGVKVLFALICIAVAEAQVQLVQSGGGLVQPGGPLRLSCTASGFSFS | | |
| Ros9d | NNYYMCWVRQAPGKGLEWVSCIYTNNIGSTWYASWAKGRFTISRD | | |
| Scaffold 22 | NSKNTLYLQMNSLRAEDTAVYYCARGIYSDSSDFIGNLWGQGTLVT | | |
| | VSSGGGGSGGGGSGGGGSGGGGSAIQMTQSPSSLSASVGDRVTITC | | |
| | QASQTIYNYLAWYQQKPGKVPELLIYKASTLASGVPSRFMGTGFGT | | |
| | DFTLTIDSLQPEDAATYYCQQYNTIINVDRTFGGGTKVEIKGGGGSA | | |
| | GVTQSPTHLIKTRGQQVTLRCSPKSGHDTVSWYQQALGOGPQFIFQ | | |
| | YYEEEERQRGNFPDRFSGHQFPNYSSELNVNALLLGDSALYLCASSL | | |
| | GQTNYGYTFGSGTRLTVVEDLKNVFPPEVAVFEPSKAEISRTQKATL | | |
| | VCLATGFYPPHVELSWWVNGKEVHDGVCTDPQPLKEQPALQDSRY | | |
| | ALSSRLRVSATFWQDPRNHFRCQVQFYGLSENDEWTQDRAKPVTQI | | |
| | VSAEAWGRADCGGGGSGLNDIFEAQKIEWHEGGGGSGGGGSLPET | | |
| | GG | | |
| | | MMGVKVLFALICIAVAEAD | 756 |
| | Human | LAKTTQPISVDSYEGQEVNITCSHNNIATNDYITWYQQF | 787 |
| | TCR-alpha | PSQGPRFIIQGYKTKVTNEVASLFIPADRKSSTLSLPRVS | |
| | chain | LSDTAVYYCLVGDMDQAGTALIFGKGTTLSVSSDYIQN | |
| | | PDPAVYQLRDSKSSDKFVCLFTDFDSQIQVSQSKDSDV | |
| | | YITDKCVLDMRSMDFKSNSAVAWSQKSDFTCANAFQN | |
| | | SIIPEDTFFPSPESSC | |
| | HIS tag+ | GSGHHHHHHHHHHRIRRSGSGVKQTLNFDLLKLAGDV | 788 |
| | cleavable | ESNPGP | |
| | peptide | | |
| | Linker | | |
| | Second | MMGVKVLFALICIAVAEA | 668 |
| | signal | | |
| | peptide | | |
| | Anti-CD3 | QVQLVQSGGGLVQPGGPLRLSCTASGFSFSNNYYMCW | 779 |
| | VH | VRQAPGKGLEWVSCIYINNIGSTWYASWAKGRFTISRD | |
| | | NSKNTLYLQMNSLRAEDTAVYYCARGIYSDSSDFIGNL | |
| | domain | WGQGTLVTVSS | |
| | Linker | GGGGSGGGGSGGGGSGGGGS | 760 |
| | Anti-CD3 | AIQMTQSPSSLSASVGDRVTITCQASQTIYNYLAWYQQ | 789; |
| | VL domain | KPGKVPELLIYKASTLASGVPSRFMGTGFGTDFTLTIDS | alter- |
| | | LQPEDAATYYCQQYNTIINVDRTFGGGTKVEIK | natively 778 |
| | Linker | GGGGS | 683 |
| | TCR beta | AGVTQSPTHLIKTRGQQVTLRCSPKSGHDTVSWYQQAL | 790 |
| | chain | GQGPQFIFQYYEEEERQRGNFPDRFSGHQFPNYSSELNV | |
| | | NALLLGDSALYLCASSLGQTNYGYTFGSGTRLTVVEDL | |
| | | KNVFPPEVAVFEPSKAEISRTQKATLVCLATGFYPPHVE | |
| | | LSWWVNGKEVHDGVCTDPQPLKEQPALQDSRYALSSR | |
| | | LRVSATFWQDPRNHFRCQVQFYGLSENDEWTQDRAKP | |
| | | VTQIVSAEAWGRADC | |
| | linker- | GGGGSGLNDIFEAQKIEWHEGGGGSGGGGSLPETGG | 686 |
| | BAP- | | |
| | LPETG tag | | |

### Example 7. Functional Assay of the Constructs in Table 9

FIG. 23 shows a diagrammatic view of the structure (left) and amino acid sequence (bottom) of the construct Ros9a Scaffold 22. The anti-CD3 arm is based on TR66-VHVL construct. FIG. 24 shows a diagrammatic view of the structure (left) and amino acid sequence (bottom) of the construct Ros9a Scaffold 22. The anti-CD3 arm is based on TR66-VLVH construct. FIG. 25 shows a diagrammatic view of the structure (left) and amino acid sequence (bottom) of the construct Ros9a Scaffold 22. The anti-CD3 arm is based on 210112-VHVL construct. FIG. 26 shows the a diagrammatic view of the structure (left) and amino acid sequence (bottom) of the construct Ros9a Scaffold 22. The anti-CD3 arm is based on 210112-VLVH construct. FIG. 27 shows the a diagrammatic view of the structure (left) and amino acid sequence (bottom) of the construct Ros9a Scaffold 22. The anti-CD3 arm is based on 210112-VLVH construct. The anti-CD3 arm is based on TR66-VHVL construct. FIG. 28 shows a diagrammatic view of the structure (left) and amino acid sequence (bottom) of the construct Ros9a Scaffold 22. The anti-CD3 arm is based on 210112-VLVH construct. The anti-CD3 arm is based on 210112-VHVL construct. The functional assays are described below.

In an exemplary assay shown as a schematic of a workflow in FIG. 22, the ability of the sTCRs built on Scaffold 22 was assayed. The target cells were A375 expressing GFP, the mutated RAS peptide and the HLA that presents the peptide (HLAC*08:02). Purified sTCR products were added to culture at the concentrations indicated for assaying T cell activation and cytotoxicity, measured by loss of GFP positive target cells compared to untreated control. As shown in FIGS. 23-28, all but the Ros9a construct with TR66 VL-VH anti-CD3 arm showed good dose dependent increase in cytotoxicity. This was further validated with endogenous peptide and HLA presentations by target HPAC tumor cells treated with Ros9a Scaffold 22 sTCR constructs in presence of T cells. FIG. 29 shows a scheme of workflow for testing the sTCRs, using donor cells. This in vitro T cell cytotoxicity assay facilitates high throughput testing and nomination of TCRs. FIGS. 30-33 shows imaging and quantitative assessments of apoptotic target cells at different peptide concentrations. However, as shown in FIG. 33 and FIG. 34, when the donor's T cells that were rested for shorter period than in the protocol for FIGS. 30-32, cell cytotoxicity was observed at even lower doses.

TABLE 10
Provided herein are exemplary bispecific sTCRs under experimentation.
Several of these sTCRs are structurally exemplified in diagrams depicted in FIGS. 16-21.

| | AMINO ACID SEQUENCE | |
|---|---|---|
| Scaffold | (Index: TRAV or TRAV+ TRAC where both present, bold; TRBV or | |
| number | TRBV + TRBC where both present, underlined; anti-CD3-VHH (SEQ ID | SEQ |
| (name) | NO: 615), bold + italic) | ID NO |
| Scaffold | MMGVKVLFALICIAVAEASQFRVSPLDRTWNLGETVELKCQVLLS | 791 |
| 24 | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSGGGGSGGGGSLAKTTQPISVDSYEGQEVNITCSHNNIAT | |
| | NDYITWYQQFPSQGPRFIIQGYKTKVTNEVASLFIPADRKSSTLS | |
| | LPRVSLSDTAVYYCLVGDMDQAGTALIFGKGTTLSVSSDYIQNP | |
| | DPAVYQLRDSKSSDKFVCLFTDFDSQIQVSQSKDSDVYITDKCV | |
| | LDMRSMDFKSNSAVAWSQKSDFTCANAFQNSIIPEDTFFPSPESS | |
| | CGSGHHHHHHHHHHRIRRSGSGVKQTLNFDLLKLAGDVESNPGPM | |
| | MGVKVLFALICIAVAEAAIQMTQSPSSLSASVGDRVTITCRASQDIRNY | |
| | LNWYQQKPGKAPKLLIYYTSRLESGVPSRFSGSGSGTDYTLTISSLQPED | |
| | FATYYCQQGNTLPWTFGQGTKVEIKGGGGSGGGGSGGGGSGGGGSG | |
| | GGSEVQLVESGGGLVQPGGSLRLSCAASGYSFTGYTMNWVRQAPGKGL | |
| | EWVALINPYKGVSTYNQKFKDRFTISVDKSKNTAYLQMNSLRAEDTAVY | |
| | YCARSGYYGDSDWYFDVWGQGTLVTVSSGGGGSAGVTQSPTHLIKTR | |
| | GQQVTLRCSPKSGHDTVSWYQQALGQGPQFIFQYYEEEERQRGNF | |
| | PDRFSGHQFPNYSSELNVNALLLGDSALYLCASSLGEGRVDGYTFG | |
| | SGTRLTVVEDLKNVFPPEVAVFEPSKAEISRTQKATLVCLATGFYPP | |
| | HVELSWWVNGKEVHDGVCTDPQPLKEQPALQDSRYALSSRLRVS | |
| | ATFWQDPRNHFRCQVQFYGLSENDEWTQDRAKPVTQIVSAEAWG | |
| | RADCGGGGSGLNDIFEAQKIEWHEGGGGSGGGGSLPETGG* | |
| 25 | MMGVKVLFALICIAVAEASQFRVSPLDRTWNLGETVELKCQVLLS | 792 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAKPTTTPAPRPPTPAPG | |
| | GGGSGGGGSGGGGSLAKTTQPISVDSYEGQEVNITCSHNNIATND | |
| | YITWYQQFPSQGPRFIIQGYKTKVTNEVASLFIPADRKSSTLSLP | |
| | RVSLSDTAVYYCLVGDMDQAGTALIFGKGTTLSVSSDYIQNPDP | |
| | AVYQLRDSKSSDKFVCLFTDFDSQIQVSQSKDSDVYITDKCVLD | |
| | MRSMDFKSNSAVAWSQKSDFTCANAFQNSIIPEDTFFPSPESSCG | |
| | SGHHHHHHHHHHRIRRSGSGVKQTLNFDLLKLAGDVESNPGPMM | |
| | GVKVLFALICIAVAEAAIQMTQSPSSLSASVGDRVTITCRASQDIRNYLN | |
| | WYQQKPGKAPKLLIYYTSRLESGVPSRFSGSGSGTDYTLTISSLQPEDFA | |
| | TYYCQQGNTLPWTFGQGTKVEIKGGGGSGGGGSGGGGSGGGGSGGG | |
| | SEVQLVESGGGLVQPGGSLRLSCAASGYSFTGYTMNWVRQAPGKGLEW | |
| | VALINPYKGVSTYNQKFKDRFTISVDKSKNTAYLQMNSLRAEDTAVYYCA | |
| | RSGYYGDSDWYFDVWGQGTLVTVSSGGGGSAGVTQSPTHLIKTRGQ | |
| | QVTLRCSPKSGHDTVSWYQQALGQGPQFIFQYYEEEERQRGNFPD | |
| | RFSGHQFPNYSSELNVNALLLGDSALYLCASSLGEGRVDGYTFGSG | |
| | TRLTVVEDLKNVFPPEVAVFEPSKAEISRTQKATLVCLATGFYPPHV | |
| | ELSWWVNGKEVHDGVCTDPQPLKEQPALQDSRYALSSRLRVSATF | |
| | WQDPRNHFRCQVQFYGLSENDEWTQDRAKPVTQIVSAEAWGRAD | |
| | CGGGGSGLNDIFEAQKIEWHEGGGGSGGGGSLPETGG* | |
| 26 | MMGVKVLFALICIAVAEASQFRVSPLDRTWNLGETVELKCQVLLS | 793 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSGGGGSGGGGSLAKTTQPISVDSYEGQEVNITCS | |
| | HNNIATNDYITWYQQFPSQGPRFIIQGYKTKVTNEVASLFIPADR | |
| | KSSTLSLPRVSLSDTAVYYCLVGDMDQAGTALIFGKGTTLSVSS | |
| | DYIQNPDPAVYQLRDSKSSDKFVCLFTDFDSQIQVSQSKDSDVYI | |
| | TDKCVLDMRSMDFKSNSAVAWSQKSDFTCANAFQNSIIPEDTFF | |
| | PSPESSCGSGHHHHHHHHHHRIRRSGSGVKQTLNFDLLKLAGDVE | |
| | SNPGPMMGVKVLFALICIAVAEAAIQMTQSPSSLSASVGDRVTITCRAS | |
| | QDIRNYLNWYQQKPGKAPKLLIYYTSRLESGVPSRFSGSGSGTDYTLTISS | |
| | LQPEDFATYYCQQGNTLPWTFGQGTKVEIKGGGGSGGGGSGGGGSG | |
| | GGGSGGGSEVQLVESGGGLVQPGGSLRLSCAASGYSFTGYTMNWVRQ | |
| | APGKGLEWVALINPYKGVSTYNQKFKDRFTISVDKSKNTAYLQMNSLRA | |
| | EDTAVYYCARSGYYGDSDWYFDVWGQGTLVTVSSGGGGSAGVTQSPT | |
| | HLIKTRGQQVTLRCSPKSGHDTVSWYQQALGQGPQFIFQYYEEEER | |
| | QRGNFPDRFSGHQFPNYSSELNVNALLLGDSALYLCASSLGEGRVD | |
| | GYTFGSGTRLTVVEDLKNVFPPEVAVFEPSKAEISRTQKATLVCLAT | |
| | GFYPPHVELSWWVNGKEVHDGVCTDPQPLKEQPALQDSRYALSSR | |
| | LRVSATFWQDPRNHFRCQVQFYGLSENDEWTQDRAKPVTQIVSAE | |
| | AWGRADCGGGGSGLNDIFEAQKIEWHEGGGGSGGGGSLPETGG | |
| 27 | MMGVKVLFALICIAVAEASQFRVSPLDRTWNLGETVELKCQVLLS | 794 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAQPTKKSTL | |
| | KKRVGGGGSGGGGSGGGGSLAKTTQPISVDSYEGQEVNITCSHN | |
| | NIATNDYITWYQQFPSQGPRFIIQGYKTKVTNEVASLFIPADRKS | |
| | STLSLPRVSLSDTAVYYCLVGDMDQAGTALIFGKGTTLSVSSDY | |
| | IQNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAAIQMTQSPSSLSASVGDRVTITCRASQ | |
| | DIRNYLNWYQQKPGKAPKLLIYYTSRLESGVPSRFSGSGSGTDYTLTISSL | |
| | QPEDFATYYCQQGNTLPWTFGQGTKVEIKGGGGSGGGGSGGGGSGG | |
| | GGSGGGSEVQLVESGGGLVQPGGSLRLSCAASGYSFTGYTMNWVRQAP | |
| | GKGLEWVALINPYKGVSTYNQKFKDRFTISVDKSKNTAYLQMNSLRAED | |
| | TAVYYCARSGYYGDSDWYFDVWGQGTLVTVSSGGGGSAGVTQSPTHL | |
| | IKTRGQQVTLRCSPKSGHDTVSWYQQALGQGPQFIFQYYEEEERQR | |
| | GNFPDRFSGHQFPNYSSELNVNALLLGDSALYLCASSLGEGRVDGY | |
| | TFGSGTRLTVVEDLKNVFPPEVAVFEPSKAEISRTQKATLVCLATGF | |
| | YPPHVELSWWVNGKEVHDGVCTDPQPLKEQPALQDSRYALSSRLR | |
| | VSATFWQDPRNHFRCQVQFYGLSENDEWTQDRAKPVTQIVSAEA | |
| | WGRADCGGGGSGLNDIFEAQKIEWHEGGGGSGGGGSLPETGG | |
| 28 | MMGVKVLFALICIAVAEADLAKTTQPISVDSYEGQEVNITCSHNN | 795 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEASQFRVSPLDRTWNLGETVELKCQV | |
| | LLSNPTSGCSWLFQPRGAAASPTFLLYLSQNKPKAAEGLDTQRFSG | |
| | KRLGDTFVLTLSDFRRENEGCYFCSALSNSIMYFSHFVPVFLPAGGG | |
| | GSGGGGSGGGGSSQFRVSPLDRTWNLGETVELKCQVLLSNPTSGCS | |
| | WLFQPRGAAASPTFLLYLSQNKPKAAEGLDTQRFSGKRLGDTFVLT | |
| | LSDFRRENEGCYFCSALSNSIMYFSHFVPVFLPAGGGGSGGGGSGG | |
| | GGSAIQMTQSPSSLSASVGDRVTITCRASQDIRNYLNWYQQKPGKAPKL | |
| | LIYYTSRLESGVPSRFSGSGSGTDYTLTISSLQPEDFATYYCQQGNTLPWT | |
| | FGQGTKVEIKGGGGSGGGGSGGGGSGGGGSGGGSEVQLVESGGGLV | |
| | QPGGSLRLSCAASGYSFTGYTMNWVRQAPGKGLEWVALINPYKGVSTY | |
| | NQKFKDRFTISVDKSKNTAYLQMNSLRAEDTAVYYCARSGYYGDSDWY | |
| | FDVWGQGTLVTVSSGGGGSAGVTQSPTHLIKTRGQQVTLRCSPKSG | |
| | HDTVSWYQQALGQGPQFIFQYYEEEERQRGNFPDRFSGHQFPNYSS | |
| | ELNVNALLLGDSALYLCASSLGEGRVDGYTFGSGTRLTVVEDLKN | |
| | VFPPEVAVFEPSKAEISRTQKATLVCLATGFYPPHVELSWWVNGKE | |
| | VHDGVCTDPQPLKEQPALQDSRYALSSRLRVSATFWQDPRNHFRC | |
| | QVQFYGLSENDEWTQDRAKPVTQIVSAEAWGRADCGGGGSGLND | |
| | IFEAQKIEWHEGGGGSGGGGSLPETGG | |
| 29 | MMGVKVLFALICIAVAEADLAKTTQPISVDSYEGQEVNITCSHNN | 796 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEASQFRVSPLDRTWNLGETVELKCQV | |
| | LLSNPTSGCSWLFQPRGAAASPTFLLYLSQNKPKAAEGLDTQRFSG | |
| | KRLGDTFVLTLSDFRRENEGCYFCSALSNSIMYFSHFVPVFLPAGGG | |
| | GSGGGGSGGGGSSQFRVSPLDRTWNLGETVELKCQVLLSNPTSGCS | |
| | WLFQPRGAAASPTFLLYLSQNKPKAAEGLDTQRFSGKRLGDTFVLT | |
| | LSDFRRENEGCYFCSALSNSIMYFSHFVPVFLPAGGGGSGGGGSGG | |
| | GGSGGGGSGGGGSGGGGSAIQMTQSPSSLSASVGDRVTITCRASQDIR | |
| | NYLNWYQQKPGKAPKLLIYYTSRLESGVPSRFSGSGSGTDYTLTISSLQP | |
| | EDFATYYCQQGNTLPWTFGQGTKVEIKGGGGSGGGGSGGGGSGGGG | |
| | SGGGSEVQLVESGGGLVQPGGSLRLSCAASGYSFTGYTMNWVRQAPGK | |
| | GLEWVALINPYKGVSTYNQKFKDRFTISVDKSKNTAYLQMNSLRAEDTA | |
| | VYYCARSGYYGDSDWYFDVWGQGTLVTVSSGGGGSAGVTQSPTHLIK | |
| | TRGQQVTLRCSPKSGHDTVSWYQQALGQGPQFIFQYYEEEERQRG | |
| | NFPDRFSGHQFPNYSSELNVNALLLGDSALYLCASSLGEGRVDGYT | |
| | FGSGTRLTVVEDLKNVFPPEVAVFEPSKAEISRTQKATLVCLATGFY | |
| | PPHVELSWWVNGKEVHDGVCTDPQPLKEQPALQDSRYALSSRLRV | |
| | SATFWQDPRNHFRCQVQFYGLSENDEWTQDRAKPVTQIVSAEAW | |
| | GRADCGGGGSGLNDIFEAQKIEWHEGGGGSGGGGSLPETGG | |
| 30 | MMGVKVLFALICIAVAEADLAKTTQPISVDSYEGQEVNITCSHNN | 797 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEASQFRVSPLDRTWNLGETVELKCQV | |
| | LLSNPTSGCSWLFQPRGAAASPTFLLYLSQNKPKAAEGLDTQRFSG | |
| | KRLGDTFVLTLSDFRRENEGCYFCSALSNSIMYFSHFVPVFLPAGGG | |
| | GSGGGGSGGGGSVLQQTPAYIKVQTNKMVMLSCEAKISLSNMRIY | |
| | WLRQRQAPSSDSHHEFLALWDSAKGTIHGEEVEQEKIAVFRDASRF | |
| | ILNLTSVKPEDSGIYFCMIVGSPELTFGKGTQLSVVDFLPTTAGGGG | |
| | SGGGGSGGGGSAIQMTQSPSSLSASVGDRVTITCRASQDIRNYLNWYQQ | |
| | KPGKAPKLLIYYTSRLESGVPSRFSGSGSGTDYTLTISSLQPEDFATYYCQ | |
| | QGNTLPWTFGQGTKVEIKGGGGSGGGGSGGGGSGGGGSGGGSEVQL | |
| | VESGGGLVQPGGSLRLSCAASGYSFTGYTMNWVRQAPGKGLEWVALIN | |
| | PYKGVSTYNQKFKDRFTISVDKSKNTAYLQMNSLRAEDTAVYYCARSGY | |
| | YGDSDWYFDVWGQGTLVTVSSGGGGSAGVTQSPTHLIKTRGQQVTL | |
| | RCSPKSGHDTVSWYQQALGQGPQFIFQYYEEEERQRGNFPDRFSGH | |
| | QFPNYSSELNVNALLLGDSALYLCASSLGEGRVDGYTFGSGTRLTV | |
| | VEDLKNVFPPEVAVFEPSKAEISRTQKATLVCLATGFYPPHVELSW | |
| | WVNGKEVHDGVCTDPQPLKEQPALQDSRYALSSRLRVSATFWQDP | |
| | RNHFRCQVQFYGLSENDEWTQDRAKPVTQIVSAEAWGRADCGGG | |
| | GSGLNDIFEAQKIEWHEGGGGSGGGGSLPETGG | |
| 31 | MMGVKVLFALICIAVAEADLAKTTQPISVDSYEGQEVNITCSHNN | 798 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEASQFRVSPLDRTWNLGETVELKCQV | |
| | LLSNPTSGCSWLFQPRGAAASPTFLLYLSQNKPKAAEGLDTQRFSG | |
| | KRLGDTFVLTLSDFRRENEGCYFCSALSNSIMYFSHFVPVFLPAGGG | |
| | GSGGGGSGGGGSVLQQTPAYIKVQTNKMVMLSCEAKISLSNMRIY | |
| | WLRQRQAPSSDSHHEFLALWDSAKGTIHGEEVEQEKIAVFRDASRF | |
| | ILNLTSVKPEDSGIYFCMIVGSPELTFGKGTQLSVVDFLPTTAGGGG | |
| | SGGGGSGGGGSGGGGSGGGGSGGGGSAIQMTQSPSSLSASVGDRVTI | |
| | TCRASQDIRNYLNWYQQKPGKAPKLLIYYTSRLESGVPSRFSGSGSGTDY | |
| | TLTISSLQPEDFATYYCQQGNTLPWTFGQGTKVEIKGGGGSGGGGSGG | |
| | GGSGGGGSGGGSEVQLVESGGGLVQPGGSLRLSCAASGYSFTGYTMN | |
| | WVRQAPGKGLEWVALINPYKGVSTYNQKFKDRFTISVDKSKNTAYLQM | |
| | NSLRAEDTAVYYCARSGYYGDSDWYFDVWGQGTLVTVSSGGGGSAGV | |
| | TQSPTHLIKTRGQQVTLRCSPKSGHDTVSWYQQALGQGPQFIFQYY | |
| | EEEERQRGNFPDRFSGHQFPNYSSELNVNALLLGDSALYLCASSLG | |
| | EGRVDGYTFGSGTRLTVVEDLKNVFPPEVAVFEPSKAEISRTQKAT | |
| | LVCLATGFYPPHVELSWWVNGKEVHDGVCTDPQPLKEQPALQDSR | |
| | YALSSRLRVSATFWQDPRNHFRCQVQFYGLSENDEWTQDRAKPVT | |
| | QIVSAEAWGRADCGGGGSGLNDIFEAQKIEWHEGGGGSGGGGSLP | |
| | ETGG | |
| 36 | MMGVKVLFALICIAVAEADLAKTTQPISVDSYEGQEVNITCSHNN | 799 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAAIQMTQSPSSLSASVGDRVTITCRASQ | |
| | DIRNYLNWYQQKPGKAPKLLIYYTSRLESGVPSRFSGSGSGTDYTLTISSL | |
| | QPEDFATYYCQQGNTLPWTFGQGTKVEIKGGGGSGGGGSGGGGSGG | |
| | GGSGGGSEVQLVESGGGLVQPGGSLRLSCAASGYSFTGYTMNWVRQAP | |
| | GKGLEWVALINPYKGVSTYNQKFKDRFTISVDKSKNTAYLQMNSLRAED | |
| | TAVYYCARSGYYGDSDWYFDVWGQGTLVTVSSGGGGSGGGGSGGGG | |
| | SAIQMTQSPSSLSASVGDRVTITCRASQDIRNYLNWYQQKPGKAPKLLIY | |
| | YTSRLESGVPSRFSGSGSGTDYTLTISSLQPEDFATYYCQQGNTLPWTFG | |
| | QGTKVEIKGGGGSGGGGSGGGGSGGGGSGGGSEVQLVESGGGLVQP | |
| | GGSLRLSCAASGYSFTGYTMNWVRQAPGKGLEWVALINPYKGVSTYNQ | |
| | KFKDRFTISVDKSKNTAYLQMNSLRAEDTAVYYCARSGYYGDSDWYFDV | |
| | WGQGTLVTVSSGGGGSAGVTQSPTHLIKTRGQQVTLRCSPKSGHDT | |
| | VSWYQQALGQGPQFIFQYYEEEERQRGNFPDRFSGHQFPNYSSELN | |
| | VNALLLGDSALYLCASSLGEGRVDGYTFGSGTRLTVVEDLKNVFPP | |
| | EVAVFEPSKAEISRTQKATLVCLATGFYPPHVELSWWVNGKEVHD | |
| | GVCTDPQPLKEQPALQDSRYALSSRLRVSATFWQDPRNHFRCQVQ | |
| | FYGLSENDEWTQDRAKPVTQIVSAEAWGRADCGGGGSGLNDIFEA | |
| | QKIEWHEGGGGSGGGGSLPETGG | |
| 37 | MMGVKVLFALICIAVAEAAIQMTQSPSSLSASVGDRVTITCRASQDIRN | 800 |
| | YLNWYQQKPGKAPKLLIYYTSRLESGVPSRFSGSGSGTDYTLTISSLQPE | |
| | DFATYYCQQGNTLPWTFGQGTKVEIKGGGGSGGGGSGGGGSGGGGS | |
| | GGGSEVQLVESGGGLVQPGGSLRLSCAASGYSFTGYTMNWVRQAPGK | |
| | GLEWVALINPYKGVSTYNQKFKDRFTISVDKSKNTAYLQMNSLRAEDTA | |
| | VYYCARSGYYGDSDWYFDVWGQGTLVTVSSGGGGSLAKTTQPISVDS | |
| | YEGQEVNITCSHNNIATNDYITWYQQFPSQGPRFIIQGYKTKVT | |
| | NEVASLFIPADRKSSTLSLPRVSLSDTAVYYCLVGDMDQAGTAL | |
| | IFGKGTTLSVSSDYIQNPDPAVYQLRDSKSSDKFVCLFTDFDSQI | |
| | QVSQSKDSDVYITDKCVLDMRSMDFKSNSAVAWSQKSDFTCAN | |
| | AFQNSIIPEDTFFPSPESSCGSGHHHHHHHHHHRIRRSGSGVKQTL | |
| | NFDLLKLAGDVESNPGPMMGVKVLFALICIAVAEAAIQMTQSPSSLS | |
| | ASVGDRVTITCRASQDIRNYLNWYQQKPGKAPKLLIYYTSRLESGVPSRF | |
| | SGSGSGTDYTLTISSLQPEDFATYYCQQGNTLPWTFGQGTKVEIKGGGG | |
| | SGGGGSGGGGSGGGGSGGGSEVQLVESGGGLVQPGGSLRLSCAASGY | |
| | SFTGYTMNWVRQAPGKGLEWVALINPYKGVSTYNQKFKDRFTISVDKS | |
| | KNTAYLQMNSLRAEDTAVYYCARSGYYGDSDWYFDVWGQGTLVTVSSG | |
| | GGGSAGVTQSPTHLIKTRGQQVTLRCSPKSGHDTVSWYQQALGQG | |
| | PQFIFQYYEEEERQRGNFPDRFSGHQFPNYSSELNVNALLLGDSALY | |
| | LCASSLGEGRVDGYTFGSGTRLTVVEDLKNVFPPEVAVFEPSKAEIS | |
| | RTQKATLVCLATGFYPPHVELSWWVNGKEVHDGVCTDPQPLKEQ | |
| | PALQDSRYALSSRLRVSATFWQDPRNHFRCQVQFYGLSENDEWTQ | |
| | DRAKPVTQIVSAEAWGRADCGGGGSGLNDIFEAQKIEWHEGGGGS | |
| | GGGGSLPETGG | |
| 38 | MMGVKVLFALICIAVAEADLAKTTQPISVDSYEGQEVNITCSHNN | 801 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGGGGSGGGGSGGGGSQVQLVESGGGLVQAGGSLRLSCAAS | |
| | GLTFSSYGMAWFRRALGREREFVGAIRPGTIPYYSESVKGRFTVSK | |
| | DNAKNTVSLQMNSLKPEDTAVYYCAAVRDYVGMPYYSGSAYEY | |
| | WGQGTQVTVSSGSGHHHHHHHHHHRIRRSGSGVKQTLNFDLLKL | |
| | AGDVESNPGPMMGVKVLFALICIAVAEAAIQMTQSPSSLSASVGDRV | |
| | TITCRASQDIRNYLNWYQQKPGKAPKLLIYYTSRLESGVPSRFSGSGSGT | |
| | DYTLTISSLQPEDFATYYCQQGNTLPWTFGQGTKVEIKGGGGSGGGGS | |
| | GGGGSGGGGSGGGSEVQLVESGGGLVQPGGSLRLSCAASGYSFTGYT | |
| | MNWVRQAPGKGLEWVALINPYKGVSTYNQKFKDRFTISVDKSKNTAYL | |
| | QMNSLRAEDTAVYYCARSGYYGDSDWYFDVWGQGTLVTVSSGGGGSA | |
| | GVTQSPTHLIKTRGQQVTLRCSPKSGHDTVSWYQQALGQGPQFIFQ | |
| | YYEEEERQRGNFPDRFSGHQFPNYSSELNVNALLLGDSALYLCASS | |
| | LGEGRVDGYTFGSGTRLTVVEDLKNVFPPEVAVFEPSKAEISRTQK | |
| | ATLVCLATGFYPPHVELSWWVNGKEVHDGVCTDPQPLKEQPALQ | |
| | DSRYALSSRLRVSATFWQDPRNHFRCQVQFYGLSENDEWTQDRAK | |
| | PVTQIVSAEAWGRADCGGGGSGLNDIFEAQKIEWHEGGGGSGGGG | |
| | SLPETGG | |
| 39 | MMGVKVLFALICIAVAEADLAKTTQPISVDSYEGQEVNITCSHNN | 802 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAAIQMTQSPSSLSASVGDRVTITCRASQ | |
| | DIRNYLNWYQQKPGKAPKLLIYYTSRLESGVPSRFSGSGSGTDYTLTISSL | |
| | QPEDFATYYCQQGNTLPWTFGQGTKVEIKGGGGSGGGGSGGGGSGG | |
| | GGSGGGSEVQLVESGGGLVQPGGSLRLSCAASGYSFTGYTMNWVRQAP | |
| | GKGLEWVALINPYKGVSTYNQKFKDRFTISVDKSKNTAYLQMNSLRAED | |
| | TAVYYCARSGYYGDSDWYFDVWGQGTLVTVSSGGGGSAGVTQSPTHL | |
| | IKTRGQQVTLRCSPKSGHDTVSWYQQALGQGPQFIFQYYEEEERQR | |
| | GNFPDRFSGHQFPNYSSELNVNALLLGDSALYLCASSLGEGRVDGY | |
| | TFGSGTRLTVVEDLKNVFPPEVAVFEPSKAEISRTQKATLVCLATGF | |
| | YPPHVELSWWVNGKEVHDGVCTDPQPLKEQPALQDSRYALSSRLR | |
| | VSATFWQDPRNHFRCQVQFYGLSENDEWTQDRAKPVTQIVSAEA | |
| | WGRADCGGGGSGGGGSGGGGSQVQLVESGGGLVQAGGSLRLSCA | |
| | ASGLTFSSYGMAWFRRALGREREFVGAIRPGTIPYYSESVKGRFTVS | |
| | KDNAKNTVSLQMNSLKPEDTAVYYCAAVRDYVGMPYYSGSAYEY | |
| | WGQGTQVTVSSGGGGSGLNDIFEAQKIEWHEGGGGSGGGGSLPET | |
| | GG | |
| 40 | MMGVKVLFALICIAVAEAQVQLVESGGGLVQAGGSLRLSCAASGL | 803 |
| | TFSSYGMAWFRRALGREREFVGAIRPGTIPYYSESVKGRFTVSKDN | |
| | AKNTVSLQMNSLKPEDTAVYYCAAVRDYVGMPYYSGSAYEYWG | |
| | QGTQVTVSSGGGGSGGGGSGGGGSGGGGSLAKTTQPISVDSYEG | |
| | QEVNITCSHNNIATNDYITWYQQFPSQGPRFIIQGYKTKVTNEV | |
| | ASLFIPADRKSSTLSLPRVSLSDTAVYYCLVGDMDQAGTALIFG | |
| | KGTTLSVSSDYIQNPDPAVYQLRDSKSSDKFVCLFTDFDSQIQVS | |
| | QSKDSDVYITDKCVLDMRSMDFKSNSAVAWSQKSDFTCANAFQ | |
| | NSIIPEDTFFPSPESSCGSGHHHHHHHHHHRIRRSGSGVKQTLNFDL | |
| | LKLAGDVESNPGPMMGVKVLFALICIAVAEAAIQMTQSPSSLSASVG | |
| | DRVTITCRASQDIRNYLNWYQQKPGKAPKLLIYYTSRLESGVPSRFSGSG | |
| | SGTDYTLTISSLQPEDFATYYCQQGNTLPWTFGQGTKVEIKGGGGSGG | |
| | GGSGGGGSGGGGSGGGSEVQLVESGGGLVQPGGSLRLSCAASGYSFT | |
| | GYTMNWVRQAPGKGLEWVALINPYKGVSTYNQKFKDRFTISVDKSKNT | |
| | AYLQMNSLRAEDTAVYYCARSGYYGDSDWYFDVWGQGTLVTVSSGGG | |
| | GSAGVTQSPTHLIKTRGQQVTLRCSPKSGHDTVSWYQQALGQGPQ | |
| | FIFQYYEEEERQRGNFPDRFSGHQFPNYSSELNVNALLLGDSALYLC | |
| | ASSLGEGRVDGYTFGSGTRLTVVEDLKNVFPPEVAVFEPSKAEISRT | |
| | QKATLVCLATGFYPPHVELSWWVNGKEVHDGVCTDPQPLKEQPA | |
| | LQDSRYALSSRLRVSATFWQDPRNHFRCQVQFYGLSENDEWTQDR | |
| | AKPVTQIVSAEAWGRADCGGGGSGLNDIFEAQKIEWHEGGGGSGG | |
| | GGSLPETGG | |
| 43 | Chain A: | 804, |
| | MMGVKVLFALICIAVAEADLAKTTQPISVDSYEGQEVNITCSHNN | 805 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSLEVLFQGPGGGGSGGGGSGGGGSDKTHTCPPCPAPELLG | |
| | GPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNK | |
| | ALPAPIEKTISKAKGQPREPQVYTLPPSREEMTKNQVSLWCLVKGF | |
| | YPSDIAVEWESNGQPENNYKTTPPVLDSDGSFFLYSKLTVDKSRWQ | |
| | QGNVFSCSVMHEALHNHYTQKSLSLSPGKGGGGSHHHHHHHHHH | |
| | (SEQ ID NO: 804) | |
| | Chain B: | |
| | MMGVKVLFALICIAVAEAAGVTQSPTHLIKTRGQQVTLRCSPKSGH | |
| | DTVSWYQQALGQGPQFIFQYYEEEERQRGNFPDRFSGHQFPNYSSE | |
| | LNVNALLLGDSALYLCASSLGEGRVDGYTFGSGTRLTVVEDLKNV | |
| | FPPEVAVFEPSKAEISRTQKATLVCLATGFYPPHVELSWWVNGKEV | |
| | HDGVCTDPQPLKEQPALQDSRYALSSRLRVSATFWQDPRNHFRCQ | |
| | VQFYGLSENDEWTQDRAKPVTQIVSAEAWGRADCGSLEVLFQGPG | |
| | GGGSGGGGSGGGGSDKTHTCPPCPAPELLGGPSVFLFPPKPKDTLE | |
| | ASRTPEVTCVVVDVSHEDPEVKFNWYVDGVEVHNAKTKPREEQY | |
| | NSTYRVVSVLTVLHQDWLNGKEYKCKVSNKALPAPIEKTISKAKG | |
| | QPREPQVYTLPPSREEMTKNQVSLSCAVKGFYPSDIAVEWESNGQP | |
| | ENNYKTTPPVLDSDGSFFLVSKLTVDKSRWQQGNVFSCSVMHEAL | |
| | HNAYTQKSLSLSPGKGGGGSGLNDIFEAQKIEWHE (SEQ ID NO: | |
| | 805) | |
| 45 | MMGVKVLFALICIAVAEALAKTTQPISVDSYEGQEVNITCSHNNI | 806 |
| | ATNDYITWYQQFPSQGPRFIIQGYKTKVTNEVASLFIPADRKSST | |
| | LSLPRVSLSDTAVYYCLVGDMDQAGTALIFGKGTTLSVSSDKRT | |
| | VAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGL | |
| | RSPVTKSFNRGEGSGHHHHHHHHHHRIRRSGSGVKQTLNFDLLKL | |
| | AGDVESNPGPMMGVKVLFALICIAVAEAAGVTQSPTHLIKTRGQQ | |
| | VTLRCSPKSGHDTVSWYQQALGQGPQFIFQYYEEEERQRGNFPDRF | |
| | SGHQFPNYSSELNVNALLLGDSALYLCASSLGEGRVDGYTFGSGTR | |
| | LTVVASTKGPSVFPLAPSSKSTSGGTAALGCLVKDYFPEPVTVSWN | |
| | SGALTSGVHTFPAVLQSSGLYSLSSVVTVPSSSLGTQTYICNVNHKP | |
| | SNTKVDKKAGSGGLNDIFEAQKIEWHE | |
| 46 | MMGVKVLFALICIAVAEALAKTTQPISVDSYEGQEVNITCSHNNI | 807 |
| | ATNDYITWYQQFPSQGPRFIIQGYKTKVTNEVASLFIPADRKSST | |
| | LSLPRVSLSDTAVYYCLVGDMDQAGTALIFGKGTTLSVSSDKRT | |
| | VAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGL | |
| | RSPVTKSFNRGECGSGHHHHHHHHHHRIRRSGSGVKQTLNFDLLK | |
| | LAGDVESNPGPMMGVKVLFALICIAVAEAAGVTQSPTHLIKTRGQQ | |
| | VTLRCSPKSGHDTVSWYQQALGQGPQFIFQYYEEEERQRGNFPDRF | |
| | SGHQFPNYSSELNVNALLLGDSALYLCASSLGEGRVDGYTFGSGTR | |
| | LTVVASTKGPSVFPLAPSSKSTSGGTAALGCLVKDYFPEPVTVSWN | |
| | SGALTSGVHTFPAVLQSSGLYSLSSVVTVPSSSLGTQTYICNVNHKP | |
| | SNTKVDKKAEPKSCGSGGLNDIFEAQKIEWHE | |
| 47 | MMGVKVLFALICIAVAEALAKTTQPISVDSYEGQEVNITCSHNNI | 808 |
| | ATNDYITWYQQFPSQGPRFIIQGYKTKVTNEVASLFIPADRKSST | |
| | LSLPRVSLSDTAVYYCLVGDMDQAGTALIFGKGTTLSVSSDAST | |
| | KGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDK | |
| | KAGSGHHHHHHHHHHRIRRSGSGVKQTLNFDLLKLAGDVESNPGP | |
| | MMGVKVLFALICIAVAEAAGVTQSPTHLIKTRGQQVTLRCSPKSGH | |
| | DTVSWYQQALGQGPQFIFQYYEEEERQRGNFPDRFSGHQFPNYSSE | |
| | LNVNALLLGDSALYLCASSLGEGRVDGYTFGSGTRLTVVKRTVAA | |
| | PSVFIFPPSDEQLKSGTASVVCLLNNFYPREAKVQWKVDNALQSGN | |
| | SQESVTEQDSKDSTYSLSSTLTLSKADYEKHKVYACEVTHQGLRSP | |
| | VTKSFNRGEGSGGLNDIFEAQKIEWHE | |
| 48 | MMGVKVLFALICIAVAEALAKTTQPISVDSYEGQEVNITCSHNNI | 809 |
| | ATNDYITWYQQFPSQGPRFIIQGYKTKVTNEVASLFIPADRKSST | |
| | LSLPRVSLSDTAVYYCLVGDMDQAGTALIFGKGTTLSVSSDAST | |
| | KGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDK | |
| | KAEPKSCGSGHHHHHHHHHHRIRRSGSGVKQTLNFDLLKLAGDVE | |
| | SNPGPMMGVKVLFALICIAVAEAAGVTQSPTHLIKTRGQQVTLRCS | |
| | PKSGHDTVSWYQQALGQGPQFIFQYYEEEERQRGNFPDRFSGHQFP | |
| | NYSSELNVNALLLGDSALYLCASSLGEGRVDGYTFGSGTRLTVVK | |
| | RTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQ | |
| | GLRSPVTKSFNRGECGSGGLNDIFEAQKIEWHE | |
| 49 | Chain A: | 810, |
| | MMGVKVLFALICIAVAEAEVQLVESGGGLVQPGGSLRLSCAASGY | 811 |
| | SFTGYTMNWVRQAPGKGLEWVALINPYKGVSTYNQKFKDRFTISV | |
| | DKSKNTAYLQMNSLRAEDTAVYYCARSGYYGDSDWYFDVWGQG | |
| | TLVTVSSGGGGSLAKTTQPISVDSYEGQEVNITCSHNNIATNDYIT | |
| | WYQQFPSQGPRFIIQGYKTKVTNEVASLFIPADRKSSTLSLPRVS | |
| | LSDTAVYYCLVGDMDQAGTALIFGKGTTLSVSSDGGGGSHHHH | |
| | HHHHHH (SEQ ID NO: 810) | |
| | Chain B: | |
| | MMGVKVLFALICIAVAEAAGVTQSPTHLIKTRGQQVTLRCSPKSGH | |
| | DTVSWYQQALGQGPQFIFQYYEEEERQRGNFPDRFSGHQFPNYSSE | |
| | LNVNALLLGDSALYLCASSLGEGRVDGYTFGSGTRLTVVGGGGSAI | |
| | QMTQSPSSLSASVGDRVTITCRASQDIRNYLNWYQQKPGKAPKLLI | |
| | YYTSRLESGVPSRFSGSGSGTDYTLTISSLQPEDFATYYCQQGNTLP | |
| | WTFGQGTKVEIKGGGGSGLNDIFEAQKIEWHE (SEQ ID NO: 811) | |
| 50 | MMGVKVLFALICIAVAEAEVQLVESGGGLVQPGGSLRLSCAASGY | 812 |
| | SFTGYTMNWVRQAPGKGLEWVALINPYKGVSTYNQKFKDRFTISV | |
| | DKSKNTAYLQMNSLRAEDTAVYYCARSGYYGDSDWYFDVWGQG | |
| | TLVTVSSGGGGSLAKTTQPISVDSYEGQEVNITCSHNNIATNDYIT | |
| | WYQQFPSQGPRFIIQGYKTKVTNEVASLFIPADRKSSTLSLPRVS | |
| | LSDTAVYYCLVGDMDQAGTALIFGKGTTLSVSSDSGSGRIRRGS | |
| | GVKQTLNFDLLKLAGDVESNPGPMMGVKVLFALICIAVAEAAGVT | |
| | QSPTHLIKTRGQQVTLRCSPKSGHDTVSWYQQALGQGPQFIFQYYE | |
| | EEERQRGNFPDRFSGHQFPNYSSELNVNALLLGDSALYLCASSLGE | |
| | GRVDGYTFGSGTRLTVVGGGGSAIQMTQSPSSLSASVGDRVTITCR | |
| | ASQDIRNYLNWYQQKPGKAPKLLIYYTSRLESGVPSRFSGSGSGTD | |
| | YTLTISSLQPEDFATYYCQQGNTLPWTFGQGTKVEIKGSGGLNDIFE | |
| | AQKIEWHEGSGHHHHHHHHHH | |
| 51 | MMGVKVLFALICIAVAEAEVQLVESGGGLVQPGGSLRLSCAASGY | 813 |
| | SFTGYTMNWVRQAPGKGLEWVALINPYKGVSTYNQKFKDRFTISV | |
| | DKSKNTAYLQMNSLRAEDTAVYYCARSGYYGDSDWYFDVWGQG | |
| | TLVTVSSGGGGSLAKTTQPISVDSYEGQEVNITCSHNNIATNDYIT | |
| | WYQQFPSQGPRFIIQGYKTKVTNEVASLFIPADRKSSTLSLPRVS | |
| | LSDTAVYYCLVGDMDQAGTALIFGKGTTLSVSSDGGGGSGGGG | |
| | SGGGGSAGVTQSPTHLIKTRGQQVTLRCSPKSGHDTVSWYQQALG | |
| | QGPQFIFQYYEEEERQRGNFPDRFSGHQFPNYSSELNVNALLLGDS | |
| | ALYLCASSLGEGRVDGYTFGSGTRLTVVGGGGSAIQMTQSPSSLSA | |
| | SVGDRVTITCRASQDIRNYLNWYQQKPGKAPKLLIYYTSRLESGVP | |
| | SRFSGSGSGTDYTLTISSLQPEDFATYYCQQGNTLPWTFGQGTKVEI | |
| | KGSGGLNDIFEAQKIEWHEGSGHHHHHHHHHH | |
| 52 | MMGVKVLFALICIAVAEADLAKTTQPISVDSYEGQEVNITCSHNN | 814 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDGG | |
| | GGSEVQLVESGGGLVQPGGSLRLSCAASGYSFTGYTMNWVRQAPG | |
| | KGLEWVALINPYKGVSTYNQKFKDRFTISVDKSKNTAYLQMNSLR | |
| | AEDTAVYYCARSGYYGDSDWYFDVWGQGTLVTVSSGGGGSGGG | |
| | GSGGGGSAIQMTQSPSSLSASVGDRVTITCRASQDIRNYLNWYQQK | |
| | PGKAPKLLIYYTSRLESGVPSRFSGSGSGTDYTLTISSLQPEDFATYY | |
| | CQQGNTLPWTFGQGTKVEIKGGGGSAGVTQSPTHLIKTRGQQVTL | |
| | RCSPKSGHDTVSWYQQALGQGPQFIFQYYEEEERQRGNFPDRFSGH | |
| | QFPNYSSELNVNALLLGDSALYLCASSLGEGRVDGYTFGSGTRLTV | |
| | VGSGGLNDIFEAQKIEWHEGSGHHHHHHHHHH | |
| 53 | MMGVKVLFALICIAVAEADLAKTTQPISVDSYEGQEVNITCSHNN | 815 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAEVQLVESGGGPVQAGGSLRLSCAAS | |
| | GRTYRGYSMAWFRQSPGKEREFVAAIVWSDGNTYYEDFVKGRFTI | |
| | SRDSAKNTLYLQMTNLKPEDTALYYCAAKIRPYIFKIAGQYDYWGQ | |
| | GTQVTVSSGGGGSAGVTQSPTHLIKTRGQQVTLRCSPKSGHDTVSW | |
| | YQQALGQGPQFIFQYYEEEERQRGNFPDRFSGHQFPNYSSELNVNA | |
| | LLLGDSALYLCASSLGEGRVDGYTFGSGTRLTVVEDLKNVFPPEVA | |
| | VFEPSKAEISRTQKATLVCLATGFYPPHVELSWWVNGKEVHDGVC | |
| | TDPQPLKEQPALQDSRYALSSRLRVSATFWQDPRNHFRCQVQFYG | |
| | LSENDEWTQDRAKPVTQIVSAEAWGRADCGGGGSGLNDIFEAQKI | |
| | EWHEGGGGSGGGGSLPETGG | |
| 55 | MMGVKVLFALICIAVAEADLAKTTQPISVDSYEGQEVNITCSHNN | 816 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAEVQLVESGGGPVQAGGSLRLSCAAS | |
| | GRTYRGYSMAWFRQSPGKEREFVAAIVWSDGNTYYEDFVKGRFTI | |
| | SRDSAKNTLYLQMTNLKPEDTALYYCAAKIRPYIFKIAGQYDYWGQ | |
| | GTQVTVSSGGGGSGGGGSAGVTQSPTHLIKTRGQQVTLRCSPKSGH | |
| | DTVSWYQQALGQGPQFIFQYYEEEERQRGNFPDRFSGHQFPNYSSE | |
| | LNVNALLLGDSALYLCASSLGEGRVDGYTFGSGTRLTVVEDLKNV | |
| | FPPEVAVFEPSKAEISRTQKATLVCLATGFYPPHVELSWWVNGKEV | |
| | HDGVCTDPQPLKEQPALQDSRYALSSRLRVSATFWQDPRNHFRCQ | |
| | VQFYGLSENDEWTQDRAKPVTQIVSAEAWGRADCGGGGSGLNDIF | |
| | EAQKIEWHEGGGGSGGGGSLPETGG | |
| 57 | MMGVKVLFALICIAVAEADLAKTTQPISVDSYEGQEVNITCSHNN | 817 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAEVQLVESGGGPVQAGGSLRLSCAAS | |
| | GRTYRGYSMAWFRQSPGKEREFVAAIVWSDGNTYYEDFVKGRFTI | |
| | SRDSAKNTLYLQMTNLKPEDTALYYCAAKIRPYIFKIAGQYDYWGQ | |
| | GTQVTVSSGGGGSGGGGSGGGGSAGVTQSPTHLIKTRGQQVTLRCS | |
| | PKSGHDTVSWYQQALGQGPQFIFQYYEEEERQRGNFPDRFSGHQFP | |
| | NYSSELNVNALLLGDSALYLCASSLGEGRVDGYTFGSGTRLTVVED | |
| | LKNVFPPEVAVFEPSKAEISRTQKATLVCLATGFYPPHVELSWWVN | |
| | GKEVHDGVCTDPQPLKEQPALQDSRYALSSRLRVSATFWQDPRNH | |
| | FRCQVQFYGLSENDEWTQDRAKPVTQIVSAEAWGRADCGGGGSG | |
| | LNDIFEAQKIEWHEGGGGSGGGGSLPETGG | |
| 101 | MMGVKVLFALICIAVAEADLAKTTQPISVDSYEGQEVNITCSHNN | 818 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAQVQLVESGGGLVQAGGSLRLSCAA | |
| | SGLTFSSYGMAWFRRALGREREFVGAIRPGTIPYYSESVKGRFTVSK | |
| | DNAKNTVSLQMNSLKPEDTAVYYCAAVRDYVGMPYYSGSAYEY | |
| | WGQGTQVTVSSGGGGSGGGGSAIQMTQSPSSLSASVGDRVTITCRAS | |
| | QDIRNYLNWYQQKPGKAPKLLIYYTSRLESGVPSRFSGSGSGTDYTLTISS | |
| | LQPEDFATYYCQQGNTLPWTFGQGTKVEIKGGGGSGGGGSGGGGSG | |
| | GGGSGGGSEVQLVESGGGLVQPGGSLRLSCAASGYSFTGYTMNWVRQ | |
| | APGKGLEWVALINPYKGVSTYNQKFKDRFTISVDKSKNTAYLQMNSLRA | |
| | EDTAVYYCARSGYYGDSDWYFDVWGQGTLVTVSSGGGGSAGVTQSPT | |
| | HLIKTRGQQVTLRCSPKSGHDTVSWYQQALGQGPQFIFQYYEEEER | |
| | QRGNFPDRFSGHQFPNYSSELNVNALLLGDSALYLCASSLGEGRVD | |
| | GYTFGSGTRLTVVEDLKNVFPPEVAVFEPSKAEISRTQKATLVCLAT | |
| | GFYPPHVELSWWVNGKEVHDGVCTDPQPLKEQPALQDSRYALSSR | |
| | LRVSATFWQDPRNHFRCQVQFYGLSENDEWTQDRAKPVTQIVSAE | |
| | AWGRADCGGGGSGLNDIFEAQKIEWHEGGGGSGGGGSLPETGG | |
| 102 | MMGVKVLFALICIAVAEADLAKTTQPISVDSYEGQEVNITCSHNN | 819 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAQVQLVESGGGLVQAGGSLRLSCAA | |
| | SGLTFSSYGMAWFRRALGREREFVGAIRPGTIPYYSESVKGRFTVSK | |
| | DNAKNTVSLQMNSLKPEDTAVYYCAAVRDYVGMPYYSGSAYEY | |
| | WGQGTQVTVSSGGGGSGGGGSEVQLVESGGGPVQAGGSLRLSCAA | |
| | SGRTYRGYSMAWFRQSPGKEREFVAAIVWSDGNTYYEDFVKGRFT | |
| | ISRDSAKNTLYLQMTNLKPEDTALYYCAAKIRPYIFKIAGQYDYWGQ | |
| | GTQVTVSSGGGGSAGVTQSPTHLIKTRGQQVTLRCSPKSGHDTVSW | |
| | YQQALGQGPQFIFQYYEEEERQRGNFPDRFSGHQFPNYSSELNVNA | |
| | LLLGDSALYLCASSLGEGRVDGYTFGSGTRLTVVEDLKNVFPPEVA | |
| | VFEPSKAEISRTQKATLVCLATGFYPPHVELSWWVNGKEVHDGVC | |
| | TDPQPLKEQPALQDSRYALSSRLRVSATFWQDPRNHERCQVQFYG | |
| | LSENDEWTQDRAKPVTQIVSAEAWGRADCGGGGSGLNDIFEAQKI | |
| | EWHEGGGGSGGGGSLPETGG | |
| 103 | MMGVKVLFALICIAVAEADLAKTTQPISVDSYEGQEVNITCSHNN | 820 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAEVQLVESGGGPVQAGGSLRLSCAAS | |
| | GRTYRGYSMAWFRQSPGKEREFVAAIVWSDGNTYYEDFVKGRFTI | |
| | SRDSAKNTLYLQMTNLKPEDTALYYCAAKIRPYIFKIAGQYDYWGQ | |
| | GTQVTVSSGGGGSGGGGSEVQLVESGGGPVQAGGSLRLSCAASGR | |
| | TYRGYSMAWFRQSPGKEREFVAAIVWSDGNTYYEDFVKGRFTISR | |
| | DSAKNTLYLQMTNLKPEDTALYYCAAKIRPYIFKIAGQYDYWGQGT | |
| | QVTVSSGGGGSAGVTQSPTHLIKTRGQQVTLRCSPKSGHDTVSWY | |
| | QQALGQGPQFIFQYYEEEERQRGNFPDRFSGHQFPNYSSELNVNAL | |
| | LLGDSALYLCASSLGEGRVDGYTFGSGTRLTVVEDLKNVFPPEVAV | |
| | FEPSKAEISRTQKATLVCLATGFYPPHVELSWWVNGKEVHDGVCT | |
| | DPQPLKEQPALQDSRYALSSRLRVSATFWQDPRNHFRCQVQFYGLS | |
| | ENDEWTQDRAKPVTQIVSAEAWGRADCGGGGSGLNDIFEAQKIEW | |
| | HEGGGGSGGGGSLPETGG | |
| 104 | MMGVKVLFALICIAVAEADLAKTTQPISVDSYEGQEVNITCSHNN | 821 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGGGGSGGGGSQVQLVESGGGLVQAGGSLRLSCAASGLTFSS | |
| | YGMAWFRRALGREREFVGAIRPGTIPYYSESVKGRFTVSKDNAKN | |
| | TVSLQMNSLKPEDTAVYYCAAVRDYVGMPYYSGSAYEYWGQGT | |
| | QVTVSSGSGHHHHHHHHHHRIRRSGSGVKQTLNFDLLKLAGDVES | |
| | NPGPMMGVKVLFALICIAVAEAEVQLVESGGGPVQAGGSLRLSCA | |
| | ASGRTYRGYSMAWFRQSPGKEREFVAAIVWSDGNTYYEDFVKGRF | |
| | TISRDSAKNTLYLQMTNLKPEDTALYYCAAKIRPYIFKIAGQYDYWG | |
| | QGTQVTVSSGGGGSAGVTQSPTHLIKTRGQQVTLRCSPKSGHDTVS | |
| | WYQQALGQGPQFIFQYYEEEERQRGNFPDRFSGHQFPNYSSELNVN | |
| | ALLLGDSALYLCASSLGEGRVDGYTFGSGTRLTVVEDLKNVFPPEV | |
| | AVFEPSKAEISRTQKATLVCLATGFYPPHVELSWWVNGKEVHDGV | |
| | CTDPQPLKEQPALQDSRYALSSRLRVSATFWQDPRNHFRCQVQFY | |
| | GLSENDEWTQDRAKPVTQIVSAEAWGRADCGGGGSGLNDIFEAQK | |
| | IEWHEGGGGSGGGGSLPETGG | |
| 105 | MMGVKVLFALICIAVAEADLAKTTQPISVDSYEGQEVNITCSHNN | 822 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGGGGSGGGGSQVQLVESGGGLVQAGGSLRLSCAASGLTFSS | |
| | YGMAWFRRALGREREFVGAIRPGTIPYYSESVKGRFTVSKDNAKN | |
| | TVSLQMNSLKPEDTAVYYCAAVRDYVGMPYYSGSAYEYWGQGT | |
| | QVTVSSGSGHHHHHHHHHHRIRRSGSGVKQTLNFDLLKLAGDVES | |
| | NPGPMMGVKVLFALICIAVAEAAGVTQSPTHLIKTRGQQVTLRCSP | |
| | KSGHDTVSWYQQALGQGPQFIFQYYEEEERQRGNFPDRFSGHQFP | |
| | NYSSELNVNALLLGDSALYLCASSLGEGRVDGYTFGSGTRLTVVED | |
| | LKNVFPPEVAVFEPSKAEISRTQKATLVCLATGFYPPHVELSWWVN | |
| | GKEVHDGVCTDPQPLKEQPALQDSRYALSSRLRVSATFWQDPRNH | |
| | FRCQVQFYGLSENDEWTQDRAKPVTQIVSAEAWGRADCGGGGSG | |
| | GGGSEVQLVESGGGPVQAGGSLRLSCAASGRTYRGYSMAWFRQSP | |
| | GKEREFVAAIVWSDGNTYYEDFVKGRFTISRDSAKNTLYLQMTNLK | |
| | PEDTALYYCAAKIRPYIFKIAGQYDYWGQGTQVTVSSGGGGSGLNDI | |
| | FEAQKIEWHEGGGGSGGGGSLPETGG | |
| 106 | MMGVKVLFALICIAVAEADLAKTTQPISVDSYEGQEVNITCSHNN | 823 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAAGVTQSPTHLIKTRGQQVTLRCSPK | |
| | SGHDTVSWYQQALGQGPQFIFQYYEEEERQRGNFPDRFSGHQFPN | |
| | YSSELNVNALLLGDSALYLCASSLGEGRVDGYTFGSGTRLTVVEDL | |
| | KNVFPPEVAVFEPSKAEISRTQKATLVCLATGFYPPHVELSWWVNG | |
| | KEVHDGVCTDPQPLKEQPALQDSRYALSSRLRVSATFWQDPRNHF | |
| | RCQVQFYGLSENDEWTQDRAKPVTQIVSAEAWGRADCGGGGSGG | |
| | GGSEVQLVESGGGPVQAGGSLRLSCAASGRTYRGYSMAWFRQSPG | |
| | KEREFVAAIVWSDGNTYYEDFVKGRFTISRDSAKNTLYLQMTNLKP | |
| | EDTALYYCAAKIRPYIFKIAGQYDYWGQGTQVTVSSGGGGSGGGGS | |
| | EVQLVESGGGPVQAGGSLRLSCAASGRTYRGYSMAWFRQSPGKER | |
| | EFVAAIVWSDGNTYYEDFVKGRFTISRDSAKNTLYLQMTNLKPEDT | |
| | ALYYCAAKIRPYIFKIAGQYDYWGQGTQVTVSSGGGGSGLNDIFEAQ | |
| | KIEWHEGGGGSGGGGSLPETGG | |
| 107 | MMGVKVLFALICIAVAEADLAKTTQPISVDSYEGQEVNITCSHNN | 824 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAAGVTQSPTHLIKTRGQQVTLRCSPK | |
| | SGHDTVSWYQQALGQGPQFIFQYYEEEERQRGNFPDRFSGHQFPN | |
| | YSSELNVNALLLGDSALYLCASSLGEGRVDGYTFGSGTRLTVVEDL | |
| | KNVFPPEVAVFEPSKAEISRTQKATLVCLATGFYPPHVELSWWVNG | |
| | KEVHDGVCTDPQPLKEQPALQDSRYALSSRLRVSATFWQDPRNHF | |
| | RCQVQFYGLSENDEWTQDRAKPVTQIVSAEAWGRADCGGGGSGG | |
| | GGSEVQLVESGGGPVQAGGSLRLSCAASGRTYRGYSMAWFRQSPG | |
| | KEREFVAAIVWSDGNTYYEDFVKGRFTISRDSAKNTLYLQMTNLKP | |
| | EDTALYYCAAKIRPYIFKIAGQYDYWGQGTQVTVSSGGGGSGGGGS | |
| | QVQLVESGGGLVQAGGSLRLSCAASGLTFSSYGMAWFRRALGREREFV | |
| | GAIRPGTIPYYSESVKGRFTVSKDNAKNTVSLQMNSLKPEDTAVYYCAAV | |
| | RDYVGMPYYSGSAYEYWGQGTQVTVSSGGGGSGLNDIFEAQKIEWHE | |
| | GGGGSGGGGSLPETGG | |
| 108 | MMGVKVLFALICIAVAEADLAKTTQPISVDSYEGQEVNITCSHNN | 825 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGSGHHHHHHHHHHRIRRSGSGVKQTLNFDLLKLAGDVESN | |
| | PGPMMGVKVLFALICIAVAEAAGVTQSPTHLIKTRGQQVTLRCSPK | |
| | SGHDTVSWYQQALGQGPQFIFQYYEEEERQRGNFPDRFSGHQFPN | |
| | YSSELNVNALLLGDSALYLCASSLGEGRVDGYTFGSGTRLTVVEDL | |
| | KNVFPPEVAVFEPSKAEISRTQKATLVCLATGFYPPHVELSWWVNG | |
| | KEVHDGVCTDPQPLKEQPALQDSRYALSSRLRVSATFWQDPRNHF | |
| | RCQVQFYGLSENDEWTQDRAKPVTQIVSAEAWGRADCGGGGSGG | |
| | GGSAIQMTQSPSSLSASVGDRVTITCRASQDIRNYLNWYQQKPGKAPKL | |
| | LIYYTSRLESGVPSRFSGSGSGTDYTLTISSLQPEDFATYYCQQGNTLPWT | |
| | FGQGTKVEIKGGGGSGGGGSGGGGSGGGGSGGGSEVQLVESGGGLV | |
| | QPGGSLRLSCAASGYSFTGYTMNWVRQAPGKGLEWVALINPYKGVSTY | |
| | NQKFKDRFTISVDKSKNTAYLQMNSLRAEDTAVYYCARSGYYGDSDWY | |
| | FDVWGQGTLVTVSSGGGGSGLNDIFEAQKIEWHEGGGGSGGGGSLP | |
| | ETGG | |
| 109 | MMGVKVLFALICIAVAEASQFRVSPLDRTWNLGETVELKCQVLLS | 826 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGSGHHHHHH | |
| | HHHHRIRRSGSGVKQTLNFDLLKLAGDVESNPGPMMGVKVLFALI | |
| | CIAVAEAEVQLVESGGGPVQAGGSLRLSCAASGRTYRGYSMAWFR | |
| | QSPGKEREFVAAIVWSDGNTYYEDFVKGRFTISRDSAKNTLYLQMT | |
| | NLKPEDTALYYCAAKIRPYIFKIAGQYDYWGQGTQVTVSSGGGGSG | |
| | GGGSEVQLVESGGGPVQAGGSLRLSCAASGRTYRGYSMAWFRQSP | |
| | GKEREFVAAIVWSDGNTYYEDFVKGRFTISRDSAKNTLYLQMTNLK | |
| | PEDTALYYCAAKIRPYIFKIAGQYDYWGQGTQVTVSSGGGGSAGVT | |
| | QSPTHLIKTRGQQVTLRCSPKSGHDTVSWYQQALGQGPQFIFQYYE | |
| | EEERQRGNFPDRFSGHQFPNYSSELNVNALLLGDSALYLCASSLGE | |
| | GRVDGYTFGSGTRLTVVEDLKNVFPPEVAVFEPSKAEISRTQKATL | |
| | VCLATGFYPPHVELSWWVNGKEVHDGVCTDPQPLKEQPALQDSR | |
| | YALSSRLRVSATFWQDPRNHERCQVQFYGLSENDEWTQDRAKPVT | |
| | QIVSAEAWGRADCGGGGSGLNDIFEAQKIEWHEGGGGSGGGGSLP | |
| | ETGG | |
| 110 | MMGVKVLFALICIAVAEASQFRVSPLDRTWNLGETVELKCQVLLS | 827 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGSG | |
| | HHHHHHHHHHRIRRSGSGVKQTLNFDLLKLAGDVESNPGPMMGV | |
| | KVLFALICIAVAEAEVQLVESGGGPVQAGGSLRLSCAASGRTYRGYS | |
| | MAWFRQSPGKEREFVAAIVWSDGNTYYEDFVKGRFTISRDSAKNT | |
| | LYLQMTNLKPEDTALYYCAAKIRPYIFKIAGQYDYWGQGTQVTVSS | |
| | GGGGSGGGGSEVQLVESGGGPVQAGGSLRLSCAASGRTYRGYSMA | |
| | WFRQSPGKEREFVAAIVWSDGNTYYEDFVKGRFTISRDSAKNTLYL | |
| | QMTNLKPEDTALYYCAAKIRPYIFKIAGQYDYWGQGTQVTVSSGGG | |
| | GSAGVTQSPTHLIKTRGQQVTLRCSPKSGHDTVSWYQQALGQGPQ | |
| | FIFQYYEEEERQRGNFPDRFSGHQFPNYSSELNVNALLLGDSALYLC | |
| | ASSLGEGRVDGYTFGSGTRLTVVEDLKNVFPPEVAVFEPSKAEISRT | |
| | QKATLVCLATGFYPPHVELSWWVNGKEVHDGVCTDPQPLKEQPA | |
| | LQDSRYALSSRLRVSATFWQDPRNHFRCQVQFYGLSENDEWTQDR | |
| | AKPVTQIVSAEAWGRADCGGGGSGLNDIFEAQKIEWHEGGGGSGG | |
| | GGSLPETGG | |
| 111 | MMGVKVLFALICIAVAEASQFRVSPLDRTWNLGETVELKCQVLLS | 828 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGSGHHHHHH | |
| | HHHHRIRRSGSGVKQTLNFDLLKLAGDVESNPGPMMGVKVLFALI | |
| | CIAVAEAQVQLVESGGGLVQAGGSLRLSCAASGLTFSSYGMAWFR | |
| | RALGREREFVGAIRPGTIPYYSESVKGRFTVSKDNAKNTVSLQMNS | |
| | LKPEDTAVYYCAAVRDYVGMPYYSGSAYEYWGQGTQVTVSSGG | |
| | GGSGGGGSEVQLVESGGGPVQAGGSLRLSCAASGRTYRGYSMAWF | |
| | RQSPGKEREFVAAIVWSDGNTYYEDFVKGRFTISRDSAKNTLYLQM | |
| | TNLKPEDTALYYCAAKIRPYIFKIAGQYDYWGQGTQVTVSSGGGGS | |
| | AGVTQSPTHLIKTRGQQVTLRCSPKSGHDTVSWYQQALGQGPQFIF | |
| | QYYEEEERQRGNFPDRFSGHQFPNYSSELNVNALLLGDSALYLCAS | |
| | SLGEGRVDGYTFGSGTRLTVVEDLKNVFPPEVAVFEPSKAEISRTQ | |
| | KATLVCLATGFYPPHVELSWWVNGKEVHDGVCTDPQPLKEQPAL | |
| | QDSRYALSSRLRVSATFWQDPRNHFRCQVQFYGLSENDEWTQDRA | |
| | KPVTQIVSAEAWGRADCGGGGSGLNDIFEAQKIEWHEGGGGSGGG | |
| | GSLPETGG | |
| 112 | MMGVKVLFALICIAVAEASQFRVSPLDRTWNLGETVELKCQVLLS | 829 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGSG | |
| | HHHHHHHHHHRIRRSGSGVKQTLNFDLLKLAGDVESNPGPMMGV | |
| | KVLFALICIAVAEAQVQLVESGGGLVQAGGSLRLSCAASGLTFSSY | |
| | GMAWFRRALGREREFVGAIRPGTIPYYSESVKGRFTVSKDNAKNT | |
| | VSLQMNSLKPEDTAVYYCAAVRDYVGMPYYSGSAYEYWGQGTQ | |
| | VTVSSGGGGSGGGGSEVQLVESGGGPVQAGGSLRLSCAASGRTYR | |
| | GYSMAWFRQSPGKEREFVAAIVWSDGNTYYEDFVKGRFTISRDSA | |
| | KNTLYLQMTNLKPEDTALYYCAAKIRPYIFKIAGQYDYWGQGTQVT | |
| | VSSGGGGSAGVTQSPTHLIKTRGQQVTLRCSPKSGHDTVSWYQQA | |
| | LGQGPQFIFQYYEEEERQRGNFPDRFSGHQFPNYSSELNVNALLLG | |
| | DSALYLCASSLGEGRVDGYTFGSGTRLTVVEDLKNVFPPEVAVFEP | |
| | SKAEISRTQKATLVCLATGFYPPHVELSWWVNGKEVHDGVCTDPQ | |
| | PLKEQPALQDSRYALSSRLRVSATFWQDPRNHERCQVQFYGLSEN | |
| | DEWTQDRAKPVTQIVSAEAWGRADCGGGGSGLNDIFEAQKIEWHE | |
| | GGGGSGGGGSLPETGG | |
| 113 | MMGVKVLFALICIAVAEASQFRVSPLDRTWNLGETVELKCQVLLS | 830 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGSGHHHHHH | |
| | HHHHRIRRSGSGVKQTLNFDLLKLAGDVESNPGPMMGVKVLFALI | |
| | CIAVAEAQVQLVESGGGLVQAGGSLRLSCAASGLTFSSYGMAWFR | |
| | RALGREREFVGAIRPGTIPYYSESVKGRFTVSKDNAKNTVSLQMNS | |
| | LKPEDTAVYYCAAVRDYVGMPYYSGSAYEYWGQGTQVTVSSGG | |
| | GGSGGGGSAIQMTQSPSSLSASVGDRVTITCRASQDIRNYLNWYQQKPG | |
| | KAPKLLIYYTSRLESGVPSRFSGSGSGTDYTLTISSLQPEDFATYYCQQGN | |
| | TLPWTFGQGTKVEIKGGGGSGGGGSGGGGSGGGGSGGGSEVQLVES | |
| | GGGLVQPGGSLRLSCAASGYSFTGYTMNWVRQAPGKGLEWVALINPYK | |
| | GVSTYNQKFKDRFTISVDKSKNTAYLQMNSLRAEDTAVYYCARSGYYGD | |
| | SDWYFDVWGQGTLVTVSSGGGGSAGVTQSPTHLIKTRGQQVTLRCS | |
| | PKSGHDTVSWYQQALGQGPQFIFQYYEEEERQRGNFPDRFSGHQFP | |
| | NYSSELNVNALLLGDSALYLCASSLGEGRVDGYTFGSGTRLTVVED | |
| | LKNVFPPEVAVFEPSKAEISRTQKATLVCLATGFYPPHVELSWWVN | |
| | GKEVHDGVCTDPQPLKEQPALQDSRYALSSRLRVSATFWQDPRNH | |
| | FRCQVQFYGLSENDEWTQDRAKPVTQIVSAEAWGRADCGGGGSG | |
| | LNDIFEAQKIEWHEGGGGSGGGGSLPETGG | |
| 114 | MMGVKVLFALICIAVAEASQFRVSPLDRTWNLGETVELKCQVLLS | 831 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGSG | |
| | HHHHHHHHHHRIRRSGSGVKQTLNFDLLKLAGDVESNPGPMMGV | |
| | KVLFALICIAVAEAQVQLVESGGGLVQAGGSLRLSCAASGLTFSSY | |
| | GMAWFRRALGREREFVGAIRPGTIPYYSESVKGRFTVSKDNAKNT | |
| | VSLQMNSLKPEDTAVYYCAAVRDYVGMPYYSGSAYEYWGQGTQ | |
| | VTVSSGGGGSGGGGSAIQMTQSPSSLSASVGDRVTITCRASQDIRNYLN | |
| | WYQQKPGKAPKLLIYYTSRLESGVPSRFSGSGSGTDYTLTISSLQPEDFA | |
| | TYYCQQGNTLPWTFGQGTKVEIKGGGGSGGGGSGGGGSGGGGSGGG | |
| | SEVQLVESGGGLVQPGGSLRLSCAASGYSFTGYTMNWVRQAPGKGLEW | |
| | VALINPYKGVSTYNQKFKDRFTISVDKSKNTAYLQMNSLRAEDTAVYYCA | |
| | RSGYYGDSDWYFDVWGQGTLVTVSSGGGGSAGVTQSPTHLIKTRGQ | |
| | QVTLRCSPKSGHDTVSWYQQALGQGPQFIFQYYEEEERQRGNFPD | |
| | RFSGHQFPNYSSELNVNALLLGDSALYLCASSLGEGRVDGYTFGSG | |
| | TRLTVVEDLKNVFPPEVAVFEPSKAEISRTQKATLVCLATGFYPPHV | |
| | ELSWWVNGKEVHDGVCTDPQPLKEQPALQDSRYALSSRLRVSATF | |
| | WQDPRNHFRCQVQFYGLSENDEWTQDRAKPVTQIVSAEAWGRAD | |
| | CGGGGSGLNDIFEAQKIEWHEGGGGSGGGGSLPETGG | |
| 115 | MMGVKVLFALICIAVAEASQFRVSPLDRTWNLGETVELKCQVLLS | 832 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGSGHHHHHH | |
| | HHHHRIRRSGSGVKQTLNFDLLKLAGDVESNPGPMMGVKVLFALI | |
| | CIAVAEAEVQLVESGGGPVQAGGSLRLSCAASGRTYRGYSMAWFR | |
| | QSPGKEREFVAAIVWSDGNTYYEDFVKGRFTISRDSAKNTLYLQMT | |
| | NLKPEDTALYYCAAKIRPYIFKIAGQYDYWGQGTQVTVSSGGGGSG | |
| | GGGSAIQMTQSPSSLSASVGDRVTITCRASQDIRNYLNWYQQKPGKAPK | |
| | LLIYYTSRLESGVPSRFSGSGSGTDYTLTISSLQPEDFATYYCQQGNTLPW | |
| | TFGQGTKVEIKGGGGSGGGGSGGGGSGGGGSGGGSEVQLVESGGGL | |
| | VQPGGSLRLSCAASGYSFTGYTMNWVRQAPGKGLEWVALINPYKGVST | |
| | YNQKFKDRFTISVDKSKNTAYLQMNSLRAEDTAVYYCARSGYYGDSDWY | |
| | FDVWGQGTLVTVSSGGGGSAGVTQSPTHLIKTRGQQVTLRCSPKSG | |
| | HDTVSWYQQALGQGPQFIFQYYEEEERQRGNFPDRFSGHQFPNYSS | |
| | ELNVNALLLGDSALYLCASSLGEGRVDGYTFGSGTRLTVVEDLKN | |
| | VFPPEVAVFEPSKAEISRTQKATLVCLATGFYPPHVELSWWVNGKE | |
| | VHDGVCTDPQPLKEQPALQDSRYALSSRLRVSATFWQDPRNHFRC | |
| | QVQFYGLSENDEWTQDRAKPVTQIVSAEAWGRADCGGGGSGLND | |
| | IFEAQKIEWHEGGGGSGGGGSLPETGG | |
| 116 | MMGVKVLFALICIAVAEASQFRVSPLDRTWNLGETVELKCQVLLS | 833 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGSG | |
| | HHHHHHHHHHRIRRSGSGVKQTLNFDLLKLAGDVESNPGPMMGV | |
| | KVLFALICIAVAEAEVQLVESGGGPVQAGGSLRLSCAASGRTYRGYS | |
| | MAWFRQSPGKEREFVAAIVWSDGNTYYEDFVKGRFTISRDSAKNT | |
| | LYLQMTNLKPEDTALYYCAAKIRPYIFKIAGQYDYWGQGTQVTVSS | |
| | GGGGSGGGGSAIQMTQSPSSLSASVGDRVTITCRASQDIRNYLNWYQQ | |
| | KPGKAPKLLIYYTSRLESGVPSRFSGSGSGTDYTLTISSLQPEDFATYYCQ | |
| | QGNTLPWTFGQGTKVEIKGGGGSGGGGSGGGGSGGGGSGGGSEVQL | |
| | VESGGGLVQPGGSLRLSCAASGYSFTGYTMNWVRQAPGKGLEWVALIN | |
| | PYKGVSTYNQKFKDRFTISVDKSKNTAYLQMNSLRAEDTAVYYCARSGY | |
| | YGDSDWYFDVWGQGTLVTVSSGGGGSAGVTQSPTHLIKTRGQQVTL | |
| | RCSPKSGHDTVSWYQQALGQGPQFIFQYYEEEERQRGNFPDRFSGH | |
| | QFPNYSSELNVNALLLGDSALYLCASSLGEGRVDGYTFGSGTRLTV | |
| | VEDLKNVFPPEVAVFEPSKAEISRTQKATLVCLATGFYPPHVELSW | |
| | WVNGKEVHDGVCTDPQPLKEQPALQDSRYALSSRLRVSATFWQDP | |
| | RNHFRCQVQFYGLSENDEWTQDRAKPVTQIVSAEAWGRADCGGG | |
| | GSGLNDIFEAQKIEWHEGGGGSGGGGSLPETGG | |
| 117 | MMGVKVLFALICIAVAEASQFRVSPLDRTWNLGETVELKCQVLLS | 834 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGSGHHHHHH | |
| | HHHHRIRRSGSGVKQTLNFDLLKLAGDVESNPGPMMGVKVLFALI | |
| | CIAVAEAAGVTQSPTHLIKTRGQQVTLRCSPKSGHDTVSWYQQAL | |
| | GQGPQFIFQYYEEEERQRGNFPDRFSGHQFPNYSSELNVNALLLGD | |
| | SALYLCASSLGEGRVDGYTFGSGTRLTVVEDLKNVFPPEVAVFEPS | |
| | KAEISRTQKATLVCLATGFYPPHVELSWWVNGKEVHDGVCTDPQP | |
| | LKEQPALQDSRYALSSRLRVSATFWQDPRNHFRCQVQFYGLSEND | |
| | EWTQDRAKPVTQIVSAEAWGRADCGGGGSGGGGSAIQMTQSPSSLS | |
| | ASVGDRVTITCRASQDIRNYLNWYQQKPGKAPKLLIYYTSRLESGVPSRF | |
| | SGSGSGTDYTLTISSLQPEDFATYYCQQGNTLPWTFGQGTKVEIKGGGG | |
| | SGGGGSGGGGSGGGGSGGGSEVQLVESGGGLVQPGGSLRLSCAASGY | |
| | SFTGYTMNWVRQAPGKGLEWVALINPYKGVSTYNQKFKDRFTISVDKS | |
| | KNTAYLQMNSLRAEDTAVYYCARSGYYGDSDWYFDVWGQGTLVTVSSG | |
| | GGGSGGGGSEVQLVESGGGPVQAGGSLRLSCAASGRTYRGYSMAW | |
| | FRQSPGKEREFVAAIVWSDGNTYYEDFVKGRFTISRDSAKNTLYLQ | |
| | MTNLKPEDTALYYCAAKIRPYIFKIAGQYDYWGQGTQVTVSSGGGG | |
| | SGLNDIFEAQKIEWHEGGGGSGGGGSLPETGG | |
| 118 | MMGVKVLFALICIAVAEASQFRVSPLDRTWNLGETVELKCQVLLS | 835 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGSG | |
| | HHHHHHHHHHRIRRSGSGVKQTLNFDLLKLAGDVESNPGPMMGV | |
| | KVLFALICIAVAEAAGVTQSPTHLIKTRGQQVTLRCSPKSGHDTVS | |
| | WYQQALGQGPQFIFQYYEEEERQRGNFPDRFSGHQFPNYSSELNVN | |
| | ALLLGDSALYLCASSLGEGRVDGYTFGSGTRLTVVEDLKNVFPPEV | |
| | AVFEPSKAEISRTQKATLVCLATGFYPPHVELSWWVNGKEVHDGV | |
| | CTDPQPLKEQPALQDSRYALSSRLRVSATFWQDPRNHFRCQVQFY | |
| | GLSENDEWTQDRAKPVTQIVSAEAWGRADCGGGGSGGGGSAIQMT | |
| | QSPSSLSASVGDRVTITCRASQDIRNYLNWYQQKPGKAPKLLIYYTSRLES | |
| | GVPSRFSGSGSGTDYTLTISSLQPEDFATYYCQQGNTLPWTFGQGTKVE | |
| | IKGGGGSGGGGSGGGGSGGGGSGGGSEVQLVESGGGLVQPGGSLRLS | |
| | CAASGYSFTGYTMNWVRQAPGKGLEWVALINPYKGVSTYNQKFKDRFT | |
| | ISVDKSKNTAYLQMNSLRAEDTAVYYCARSGYYGDSDWYFDVWGQGTL | |
| | VTVSSGGGGSGGGGSEVQLVESGGGPVQAGGSLRLSCAASGRTYRG | |
| | YSMAWFRQSPGKEREFVAAIVWSDGNTYYEDFVKGRFTISRDSAK | |
| | NTLYLQMTNLKPEDTALYYCAAKIRPYIFKIAGQYDYWGQGTQVTV | |
| | SSGGGGSGLNDIFEAQKIEWHEGGGGSGGGGSLPETGG | |
| 119 | MMGVKVLFALICIAVAEASQFRVSPLDRTWNLGETVELKCQVLLS | 836 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGSGHHHHHH | |
| | HHHHRIRRSGSGVKQTLNFDLLKLAGDVESNPGPMMGVKVLFALI | |
| | CIAVAEAAGVTQSPTHLIKTRGQQVTLRCSPKSGHDTVSWYQQAL | |
| | GQGPQFIFQYYEEEERQRGNFPDRFSGHQFPNYSSELNVNALLLGD | |
| | SALYLCASSLGEGRVDGYTFGSGTRLTVVEDLKNVFPPEVAVFEPS | |
| | KAEISRTQKATLVCLATGFYPPHVELSWWVNGKEVHDGVCTDPQP | |
| | LKEQPALQDSRYALSSRLRVSATFWQDPRNHFRCQVQFYGLSEND | |
| | EWTQDRAKPVTQIVSAEAWGRADCGGGGSGGGGSAIQMTQSPSSLS | |
| | ASVGDRVTITCRASQDIRNYLNWYQQKPGKAPKLLIYYTSRLESGVPSRF | |
| | SGSGSGTDYTLTISSLQPEDFATYYCQQGNTLPWTFGQGTKVEIKGGGG | |
| | SGGGGSGGGGSGGGGSGGGSEVQLVESGGGLVQPGGSLRLSCAASGY | |
| | SFTGYTMNWVRQAPGKGLEWVALINPYKGVSTYNQKFKDRFTISVDKS | |
| | KNTAYLQMNSLRAEDTAVYYCARSGYYGDSDWYFDVWGQGTLVTVSSG | |
| | GGGSGGGGSQVQLVESGGGLVQAGGSLRLSCAASGLTFSSYGMA | |
| | WFRRALGREREFVGAIRPGTIPYYSESVKGRFTVSKDNAKNTVSLQ | |
| | MNSLKPEDTAVYYCAAVRDYVGMPYYSGSAYEYWGQGTQVTVS | |
| | SGGGGSGLNDIFEAQKIEWHEGGGGSGGGGSLPETGG | |
| 120 | MMGVKVLFALICIAVAEASQFRVSPLDRTWNLGETVELKCQVLLS | 837 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGSG | |
| | HHHHHHHHHHRIRRSGSGVKQTLNFDLLKLAGDVESNPGPMMGV | |
| | KVLFALICIAVAEAAGVTQSPTHLIKTRGQQVTLRCSPKSGHDTVS | |
| | WYQQALGQGPQFIFQYYEEEERQRGNFPDRFSGHQFPNYSSELNVN | |
| | ALLLGDSALYLCASSLGEGRVDGYTFGSGTRLTVVEDLKNVFPPEV | |
| | AVFEPSKAEISRTQKATLVCLATGFYPPHVELSWWVNGKEVHDGV | |
| | CTDPQPLKEQPALQDSRYALSSRLRVSATFWQDPRNHFRCQVQFY | |
| | GLSENDEWTQDRAKPVTQIVSAEAWGRADCGGGGSGGGGSAIQMT | |
| | QSPSSLSASVGDRVTITCRASQDIRNYLNWYQQKPGKAPKLLIYYTSRLES | |
| | GVPSRFSGSGSGTDYTLTISSLQPEDFATYYCQQGNTLPWTFGQGTKVE | |
| | IKGGGGSGGGGSGGGGSGGGGSGGGSEVQLVESGGGLVQPGGSLRLS | |
| | CAASGYSFTGYTMNWVRQAPGKGLEWVALINPYKGVSTYNQKFKDRFT | |
| | ISVDKSKNTAYLQMNSLRAEDTAVYYCARSGYYGDSDWYFDVWGQGTL | |
| | VTVSSGGGGSGGGGSQVQLVESGGGLVQAGGSLRLSCAASGLTFSS | |
| | YGMAWFRRALGREREFVGAIRPGTIPYYSESVKGRFTVSKDNAKN | |
| | TVSLQMNSLKPEDTAVYYCAAVRDYVGMPYYSGSAYEYWGQGT | |
| | QVTVSSGGGGSGLNDIFEAQKIEWHEGGGGSGGGGSLPETGG | |
| 121 | MMGVKVLFALICIAVAEASQFRVSPLDRTWNLGETVELKCQVLLS | 838 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGSGHHHHHH | |
| | HHHHRIRRSGSGVKQTLNFDLLKLAGDVESNPGPMMGVKVLFALI | |
| | CIAVAEAAGVTQSPTHLIKTRGQQVTLRCSPKSGHDTVSWYQQAL | |
| | GQGPQFIFQYYEEEERQRGNFPDRFSGHQFPNYSSELNVNALLLGD | |
| | SALYLCASSLGEGRVDGYTFGSGTRLTVVEDLKNVFPPEVAVFEPS | |
| | KAEISRTQKATLVCLATGFYPPHVELSWWVNGKEVHDGVCTDPQP | |
| | LKEQPALQDSRYALSSRLRVSATFWQDPRNHFRCQVQFYGLSEND | |
| | EWTQDRAKPVTQIVSAEAWGRADCGGGGSGGGGSEVQLVESGGG | |
| | PVQAGGSLRLSCAASGRTYRGYSMAWFRQSPGKEREFVAAIVWSD | |
| | GNTYYEDFVKGRFTISRDSAKNTLYLQMTNLKPEDTALYYCAAKIR | |
| | PYIFKIAGQYDYWGQGTQVTVSSGGGGSGGGGSEVQLVESGGGPV | |
| | QAGGSLRLSCAASGRTYRGYSMAWFRQSPGKEREFVAAIVWSDGN | |
| | TYYEDFVKGRFTISRDSAKNTLYLQMTNLKPEDTALYYCAAKIRPYI | |
| | FKIAGQYDYWGQGTQVTVSSGGGGSGLNDIFEAQKIEWHEGGGGS | |
| | GGGGSLPETGG | |
| 122 | MMGVKVLFALICIAVAEASQFRVSPLDRTWNLGETVELKCQVLLS | 839 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGSG | |
| | HHHHHHHHHHRIRRSGSGVKQTLNFDLLKLAGDVESNPGPMMGV | |
| | KVLFALICIAVAEAAGVTQSPTHLIKTRGQQVTLRCSPKSGHDTVS | |
| | WYQQALGQGPQFIFQYYEEEERQRGNFPDRFSGHQFPNYSSELNVN | |
| | ALLLGDSALYLCASSLGEGRVDGYTFGSGTRLTVVEDLKNVFPPEV | |
| | AVFEPSKAEISRTQKATLVCLATGFYPPHVELSWWVNGKEVHDGV | |
| | CTDPQPLKEQPALQDSRYALSSRLRVSATFWQDPRNHFRCQVQFY | |
| | GLSENDEWTQDRAKPVTQIVSAEAWGRADCGGGGSGGGGSEVQL | |
| | VESGGGPVQAGGSLRLSCAASGRTYRGYSMAWFRQSPGKEREFVA | |
| | AIVWSDGNTYYEDFVKGRFTISRDSAKNTLYLQMTNLKPEDTALYY | |
| | CAAKIRPYIFKIAGQYDYWGQGTQVTVSSGGGGSGGGGSEVQLVES | |
| | GGGPVQAGGSLRLSCAASGRTYRGYSMAWFRQSPGKEREFVAAIV | |
| | WSDGNTYYEDFVKGRFTISRDSAKNTLYLQMTNLKPEDTALYYCAA | |
| | KIRPYIFKIAGQYDYWGQGTQVTVSSGGGGSGLNDIFEAQKIEWHE | |
| | GGGGSGGGGSLPETGG | |
| 123 | MMGVKVLFALICIAVAEASQFRVSPLDRTWNLGETVELKCQVLLS | 840 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGGGGSGGGG | |
| | SQVQLVESGGGLVQAGGSLRLSCAASGLTFSSYGMAWFRRALGRE | |
| | REFVGAIRPGTIPYYSESVKGRFTVSKDNAKNTVSLQMNSLKPEDT | |
| | AVYYCAAVRDYVGMPYYSGSAYEYWGQGTQVTVSSGSGHHHHH | |
| | HHHHHRIRRSGSGVKQTLNFDLLKLAGDVESNPGPMMGVKVLFAL | |
| | ICIAVAEAAGVTQSPTHLIKTRGQQVTLRCSPKSGHDTVSWYQQAL | |
| | GQGPQFIFQYYEEEERQRGNFPDRFSGHQFPNYSSELNVNALLLGD | |
| | SALYLCASSLGEGRVDGYTFGSGTRLTVVEDLKNVFPPEVAVFEPS | |
| | KAEISRTQKATLVCLATGFYPPHVELSWWVNGKEVHDGVCTDPQP | |
| | LKEQPALQDSRYALSSRLRVSATFWQDPRNHFRCQVQFYGLSEND | |
| | EWTQDRAKPVTQIVSAEAWGRADCGGGGSGGGGSEVQLVESGGG | |
| | PVQAGGSLRLSCAASGRTYRGYSMAWFRQSPGKEREFVAAIVWSD | |
| | GNTYYEDFVKGRFTISRDSAKNTLYLQMTNLKPEDTALYYCAAKIR | |
| | PYIFKIAGQYDYWGQGTQVTVSSGGGGSGLNDIFEAQKIEWHEGGG | |
| | GSGGGGSLPETGG | |
| 124 | MMGVKVLFALICIAVAEASQFRVSPLDRTWNLGETVELKCQVLLS | 841 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGGG | |
| | GSGGGGSQVQLVESGGGLVQAGGSLRLSCAASGLTFSSYGMAWFR | |
| | RALGREREFVGAIRPGTIPYYSESVKGRFTVSKDNAKNTVSLQMNS | |
| | LKPEDTAVYYCAAVRDYVGMPYYSGSAYEYWGQGTQVTVSSGSG | |
| | HHHHHHHHHHRIRRSGSGVKQTLNFDLLKLAGDVESNPGPMMGV | |
| | KVLFALICIAVAEAAGVTQSPTHLIKTRGQQVTLRCSPKSGHDTVS | |
| | WYQQALGQGPQFIFQYYEEEERQRGNFPDRFSGHQFPNYSSELNVN | |
| | ALLLGDSALYLCASSLGEGRVDGYTFGSGTRLTVVEDLKNVFPPEV | |
| | AVFEPSKAEISRTQKATLVCLATGFYPPHVELSWWVNGKEVHDGV | |
| | CTDPQPLKEQPALQDSRYALSSRLRVSATFWQDPRNHFRCQVQFY | |
| | GLSENDEWTQDRAKPVTQIVSAEAWGRADCGGGGSGGGGSEVQL | |
| | VESGGGPVQAGGSLRLSCAASGRTYRGYSMAWFRQSPGKEREFVA | |
| | AIVWSDGNTYYEDFVKGRFTISRDSAKNTLYLQMTNLKPEDTALYY | |
| | CAAKIRPYIFKIAGQYDYWGQGTQVTVSSGGGGSGLNDIFEAQKIEW | |
| | HEGGGGSGGGGSLPETGG | |
| 125 | MMGVKVLFALICIAVAEASQFRVSPLDRTWNLGETVELKCQVLLS | 842 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGGGGSGGGG | |
| | SQVQLVESGGGLVQAGGSLRLSCAASGLTFSSYGMAWFRRALGRE | |
| | REFVGAIRPGTIPYYSESVKGRFTVSKDNAKNTVSLQMNSLKPEDT | |
| | AVYYCAAVRDYVGMPYYSGSAYEYWGQGTQVTVSSGSGHHHHH | |
| | HHHHHRIRRSGSGVKQTLNFDLLKLAGDVESNPGPMMGVKVLFAL | |
| | ICIAVAEAAGVTQSPTHLIKTRGQQVTLRCSPKSGHDTVSWYQQAL | |
| | GQGPQFIFQYYEEEERQRGNFPDRFSGHQFPNYSSELNVNALLLGD | |
| | SALYLCASSLGEGRVDGYTFGSGTRLTVVEDLKNVFPPEVAVFEPS | |
| | KAEISRTQKATLVCLATGFYPPHVELSWWVNGKEVHDGVCTDPQP | |
| | LKEQPALQDSRYALSSRLRVSATFWQDPRNHFRCQVQFYGLSEND | |
| | EWTQDRAKPVTQIVSAEAWGRADCGGGGSGGGGSEVQLVESGGG | |
| | PVQAGGSLRLSCAASGRTYRGYSMAWFRQSPGKEREFVAAIVWSD | |
| | GNTYYEDFVKGRFTISRDSAKNTLYLQMTNLKPEDTALYYCAAKIR | |
| | PYIFKIAGQYDYWGQGTQVTVSSGGGGSGGGGSEVQLVESGGGPV | |
| | QAGGSLRLSCAASGRTYRGYSMAWFRQSPGKEREFVAAIVWSDGN | |
| | TYYEDFVKGRFTISRDSAKNTLYLQMTNLKPEDTALYYCAAKIRPYI | |
| | FKIAGQYDYWGQGTQVTVSSGGGGSGLNDIFEAQKIEWHEGGGGS | |
| | GGGGSLPETGG | |
| 126 | MMGVKVLFALICIAVAEASQFRVSPLDRTWNLGETVELKCQVLLS | 843 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGGG | |
| | GSGGGGSQVQLVESGGGLVQAGGSLRLSCAASGLTFSSYGMAWFR | |
| | RALGREREFVGAIRPGTIPYYSESVKGRFTVSKDNAKNTVSLQMNS | |
| | LKPEDTAVYYCAAVRDYVGMPYYSGSAYEYWGQGTQVTVSSGSG | |
| | HHHHHHHHHHRIRRSGSGVKQTLNFDLLKLAGDVESNPGPMMGV | |
| | KVLFALICIAVAEAAGVTQSPTHLIKTRGQQVTLRCSPKSGHDTVS | |
| | WYQQALGQGPQFIFQYYEEEERQRGNFPDRFSGHQFPNYSSELNVN | |
| | ALLLGDSALYLCASSLGEGRVDGYTFGSGTRLTVVEDLKNVFPPEV | |
| | AVFEPSKAEISRTQKATLVCLATGFYPPHVELSWWVNGKEVHDGV | |
| | CTDPQPLKEQPALQDSRYALSSRLRVSATFWQDPRNHFRCQVQFY | |
| | GLSENDEWTQDRAKPVTQIVSAEAWGRADCGGGGSGGGGSEVQL | |
| | VESGGGPVQAGGSLRLSCAASGRTYRGYSMAWFRQSPGKEREFVA | |
| | AIVWSDGNTYYEDFVKGRFTISRDSAKNTLYLQMTNLKPEDTALYY | |
| | CAAKIRPYIFKIAGQYDYWGQGTQVTVSSGGGGSGGGGSEVQLVES | |
| | GGGPVQAGGSLRLSCAASGRTYRGYSMAWFRQSPGKEREFVAAIV | |
| | WSDGNTYYEDFVKGRFTISRDSAKNTLYLQMTNLKPEDTALYYCAA | |
| | KIRPYIFKIAGQYDYWGQGTQVTVSSGGGGSGLNDIFEAQKIEWHE | |
| | GGGGSGGGGSLPETGG | |
| 127 | MMGVKVLFALICIAVAEASQFRVSPLDRTWNLGETVELKCQVLLS | 844 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGGGGSGGGG | |
| | SQVQLVESGGGLVQAGGSLRLSCAASGLTFSSYGMAWFRRALGRE | |
| | REFVGAIRPGTIPYYSESVKGRFTVSKDNAKNTVSLQMNSLKPEDT | |
| | AVYYCAAVRDYVGMPYYSGSAYEYWGQGTQVTVSSGSGHHHHH | |
| | HHHHHRIRRSGSGVKQTLNFDLLKLAGDVESNPGPMMGVKVLFAL | |
| | ICIAVAEAEVQLVESGGGPVQAGGSLRLSCAASGRTYRGYSMAWFR | |
| | QSPGKEREFVAAIVWSDGNTYYEDFVKGRFTISRDSAKNTLYLQMT | |
| | NLKPEDTALYYCAAKIRPYIFKIAGQYDYWGQGTQVTVSSGGGGSA | |
| | GVTQSPTHLIKTRGQQVTLRCSPKSGHDTVSWYQQALGQGPQFIFQ | |
| | YYEEEERQRGNFPDRFSGHQFPNYSSELNVNALLLGDSALYLCASS | |
| | LGEGRVDGYTFGSGTRLTVVEDLKNVFPPEVAVFEPSKAEISRTQK | |
| | ATLVCLATGFYPPHVELSWWVNGKEVHDGVCTDPQPLKEQPALQ | |
| | DSRYALSSRLRVSATFWQDPRNHFRCQVQFYGLSENDEWTQDRAK | |
| | PVTQIVSAEAWGRADCGGGGSGLNDIFEAQKIEWHEGGGGSGGGG | |
| | SLPETGG | |
| 128 | MMGVKVLFALICIAVAEASQFRVSPLDRTWNLGETVELKCQVLLS | 845 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGGG | |
| | GSGGGGSQVQLVESGGGLVQAGGSLRLSCAASGLTFSSYGMAWFR | |
| | RALGREREFVGAIRPGTIPYYSESVKGRFTVSKDNAKNTVSLQMNS | |
| | LKPEDTAVYYCAAVRDYVGMPYYSGSAYEYWGQGTQVTVSSGSG | |
| | HHHHHHHHHHRIRRSGSGVKQTLNFDLLKLAGDVESNPGPMMGV | |
| | KVLFALICIAVAEAEVQLVESGGGPVQAGGSLRLSCAASGRTYRGYS | |
| | MAWFRQSPGKEREFVAAIVWSDGNTYYEDFVKGRFTISRDSAKNT | |
| | LYLQMTNLKPEDTALYYCAAKIRPYIFKIAGQYDYWGQGTQVTVSS | |
| | GGGGSAGVTQSPTHLIKTRGQQVTLRCSPKSGHDTVSWYQQALGQ | |
| | GPQFIFQYYEEEERQRGNFPDRFSGHQFPNYSSELNVNALLLGDSAL | |
| | YLCASSLGEGRVDGYTFGSGTRLTVVEDLKNVFPPEVAVFEPSKAE | |
| | ISRTQKATLVCLATGFYPPHVELSWWVNGKEVHDGVCTDPQPLKE | |
| | QPALQDSRYALSSRLRVSATFWQDPRNHFRCQVQFYGLSENDEWT | |
| | QDRAKPVTQIVSAEAWGRADCGGGGSGLNDIFEAQKIEWHEGGGG | |
| | SGGGGSLPETGG | |
| 129 | MMGVKVLFALICIAVAEASQFRVSPLDRTWNLGETVELKCQVLLS | 846 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGGGGSGGGG | |
| | SQVQLVESGGGLVQAGGSLRLSCAASGLTFSSYGMAWFRRALGRE | |
| | REFVGAIRPGTIPYYSESVKGRFTVSKDNAKNTVSLQMNSLKPEDT | |
| | AVYYCAAVRDYVGMPYYSGSAYEYWGQGTQVTVSSGSGHHHHH | |
| | HHHHHRIRRSGSGVKQTLNFDLLKLAGDVESNPGPMMGVKVLFAL | |
| | ICIAVAEAEVQLVESGGGPVQAGGSLRLSCAASGRTYRGYSMAWFR | |
| | QSPGKEREFVAAIVWSDGNTYYEDFVKGRFTISRDSAKNTLYLQMT | |
| | NLKPEDTALYYCAAKIRPYIFKIAGQYDYWGQGTQVTVSSGGGGSG | |
| | GGGSEVQLVESGGGPVQAGGSLRLSCAASGRTYRGYSMAWFRQSP | |
| | GKEREFVAAIVWSDGNTYYEDFVKGRFTISRDSAKNTLYLQMTNLK | |
| | PEDTALYYCAAKIRPYIFKIAGQYDYWGQGTQVTVSSGGGGSAGVT | |
| | QSPTHLIKTRGQQVTLRCSPKSGHDTVSWYQQALGQGPQFIFQYYE | |
| | EEERQRGNFPDRFSGHQFPNYSSELNVNALLLGDSALYLCASSLGE | |
| | GRVDGYTFGSGTRLTVVEDLKNVFPPEVAVFEPSKAEISRTQKATL | |
| | VCLATGFYPPHVELSWWVNGKEVHDGVCTDPQPLKEQPALQDSR | |
| | YALSSRLRVSATFWQDPRNHFRCQVQFYGLSENDEWTQDRAKPVT | |
| | QIVSAEAWGRADCGGGGSGLNDIFEAQKIEWHEGGGGSGGGGSLP | |
| | ETGG | |
| 130 | MMGVKVLFALICIAVAEASQFRVSPLDRTWNLGETVELKCQVLLS | 847 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGGG | |
| | GSGGGGSQVQLVESGGGLVQAGGSLRLSCAASGLTFSSYGMAWFR | |
| | RALGREREFVGAIRPGTIPYYSESVKGRFTVSKDNAKNTVSLQMNS | |
| | LKPEDTAVYYCAAVRDYVGMPYYSGSAYEYWGQGTQVTVSSGSG | |
| | HHHHHHHHHHRIRRSGSGVKQTLNFDLLKLAGDVESNPGPMMGV | |
| | KVLFALICIAVAEAEVQLVESGGGPVQAGGSLRLSCAASGRTYRGYS | |
| | MAWFRQSPGKEREFVAAIVWSDGNTYYEDFVKGRFTISRDSAKNT | |
| | LYLQMTNLKPEDTALYYCAAKIRPYIFKIAGQYDYWGQGTQVTVSS | |
| | GGGGSGGGGSEVQLVESGGGPVQAGGSLRLSCAASGRTYRGYSMA | |
| | WFRQSPGKEREFVAAIVWSDGNTYYEDFVKGRFTISRDSAKNTLYL | |
| | QMTNLKPEDTALYYCAAKIRPYIFKIAGQYDYWGQGTQVTVSSGGG | |
| | GSAGVTQSPTHLIKTRGQQVTLRCSPKSGHDTVSWYQQALGQGPQ | |
| | FIFQYYEEEERQRGNFPDRFSGHQFPNYSSELNVNALLLGDSALYLC | |
| | ASSLGEGRVDGYTFGSGTRLTVVEDLKNVFPPEVAVFEPSKAEISRT | |
| | QKATLVCLATGFYPPHVELSWWVNGKEVHDGVCTDPQPLKEQPA | |
| | LQDSRYALSSRLRVSATFWQDPRNHFRCQVQFYGLSENDEWTQDR | |
| | AKPVTQIVSAEAWGRADCGGGGSGLNDIFEAQKIEWHEGGGGSGG | |
| | GGSLPETGG | |
| 131 | MMGVKVLFALICIAVAEASQFRVSPLDRTWNLGETVELKCQVLLS | 848 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGGGGSGGGG | |
| | SQVQLVESGGGLVQAGGSLRLSCAASGLTFSSYGMAWFRRALGRE | |
| | REFVGAIRPGTIPYYSESVKGRFTVSKDNAKNTVSLQMNSLKPEDT | |
| | AVYYCAAVRDYVGMPYYSGSAYEYWGQGTQVTVSSGSGHHHHH | |
| | HHHHHRIRRSGSGVKQTLNFDLLKLAGDVESNPGPMMGVKVLFAL | |
| | ICIAVAEAEVQLVESGGGPVQAGGSLRLSCAASGRTYRGYSMAWFR | |
| | QSPGKEREFVAAIVWSDGNTYYEDFVKGRFTISRDSAKNTLYLQMT | |
| | NLKPEDTALYYCAAKIRPYIFKIAGQYDYWGQGTQVTVSSGGGGSA | |
| | GVTQSPTHLIKTRGQQVTLRCSPKSGHDTVSWYQQALGQGPQFIFQ | |
| | YYEEEERQRGNFPDRFSGHQFPNYSSELNVNALLLGDSALYLCASS | |
| | LGEGRVDGYTFGSGTRLTVVEDLKNVFPPEVAVFEPSKAEISRTQK | |
| | ATLVCLATGFYPPHVELSWWVNGKEVHDGVCTDPQPLKEQPALQ | |
| | DSRYALSSRLRVSATFWQDPRNHFRCQVQFYGLSENDEWTQDRAK | |
| | PVTQIVSAEAWGRADCGGGGSGGGGSQVQLVESGGGLVQAGGSL | |
| | RLSCAASGLTFSSYGMAWFRRALGREREFVGAIRPGTIPYYSESVK | |
| | GRFTVSKDNAKNTVSLQMNSLKPEDTAVYYCAAVRDYVGMPYYS | |
| | GSAYEYWGQGTQVTVSSGGGGSGLNDIFEAQKIEWHEGGGGSGG | |
| | GGSLPETGG | |
| 132 | MMGVKVLFALICIAVAEASQFRVSPLDRTWNLGETVELKCQVLLS | 849 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGGG | |
| | GSGGGGSQVQLVESGGGLVQAGGSLRLSCAASGLTFSSYGMAWFR | |
| | RALGREREFVGAIRPGTIPYYSESVKGRFTVSKDNAKNTVSLQMNS | |
| | LKPEDTAVYYCAAVRDYVGMPYYSGSAYEYWGQGTQVTVSSGSG | |
| | HHHHHHHHHHRIRRSGSGVKQTLNFDLLKLAGDVESNPGPMMGV | |
| | KVLFALICIAVAEAEVQLVESGGGPVQAGGSLRLSCAASGRTYRGYS | |
| | MAWFRQSPGKEREFVAAIVWSDGNTYYEDFVKGRFTISRDSAKNT | |
| | LYLQMTNLKPEDTALYYCAAKIRPYIFKIAGQYDYWGQGTQVTVSS | |
| | GGGGSAGVTQSPTHLIKTRGQQVTLRCSPKSGHDTVSWYQQALGQ | |
| | GPQFIFQYYEEEERQRGNFPDRFSGHQFPNYSSELNVNALLLGDSAL | |
| | YLCASSLGEGRVDGYTFGSGTRLTVVEDLKNVFPPEVAVFEPSKAE | |
| | ISRTQKATLVCLATGFYPPHVELSWWVNGKEVHDGVCTDPQPLKE | |
| | QPALQDSRYALSSRLRVSATFWQDPRNHFRCQVQFYGLSENDEWT | |
| | QDRAKPVTQIVSAEAWGRADCGGGGSGGGGSQVQLVESGGGLVQ | |
| | AGGSLRLSCAASGLTFSSYGMAWFRRALGREREFVGAIRPGTIPYY | |
| | SESVKGRFTVSKDNAKNTVSLQMNSLKPEDTAVYYCAAVRDYVG | |
| | MPYYSGSAYEYWGQGTQVTVSSGGGGSGLNDIFEAQKIEWHEGG | |
| | GGSGGGGSLPETGG | |
| 133 | MMGVKVLFALICIAVAEASQFRVSPLDRTWNLGETVELKCQVLLS | 850 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGGGGSGGGG | |
| | SQVQLVESGGGLVQAGGSLRLSCAASGLTFSSYGMAWFRRALGRE | |
| | REFVGAIRPGTIPYYSESVKGRFTVSKDNAKNTVSLQMNSLKPEDT | |
| | AVYYCAAVRDYVGMPYYSGSAYEYWGQGTQVTVSSGSGHHHHH | |
| | HHHHHRIRRSGSGVKQTLNFDLLKLAGDVESNPGPMMGVKVLFAL | |
| | ICIAVAEAEVQLVESGGGPVQAGGSLRLSCAASGRTYRGYSMAWFR | |
| | QSPGKEREFVAAIVWSDGNTYYEDFVKGRFTISRDSAKNTLYLQMT | |
| | NLKPEDTALYYCAAKIRPYIFKIAGQYDYWGQGTQVTVSSGGGGSG | |
| | GGGSEVQLVESGGGPVQAGGSLRLSCAASGRTYRGYSMAWFRQSP | |
| | GKEREFVAAIVWSDGNTYYEDFVKGRFTISRDSAKNTLYLQMTNLK | |
| | PEDTALYYCAAKIRPYIFKIAGQYDYWGQGTQVTVSSGGGGSAGVT | |
| | QSPTHLIKTRGQQVTLRCSPKSGHDTVSWYQQALGQGPQFIFQYYE | |
| | EEERQRGNFPDRFSGHQFPNYSSELNVNALLLGDSALYLCASSLGE | |
| | GRVDGYTFGSGTRLTVVEDLKNVFPPEVAVFEPSKAEISRTQKATL | |
| | VCLATGFYPPHVELSWWVNGKEVHDGVCTDPQPLKEQPALQDSR | |
| | YALSSRLRVSATFWQDPRNHFRCQVQFYGLSENDEWTQDRAKPVT | |
| | QIVSAEAWGRADCGGGGSGGGGSQVQLVESGGGLVQAGGSLRLS | |
| | CAASGLTFSSYGMAWFRRALGREREFVGAIRPGTIPYYSESVKGRF | |
| | TVSKDNAKNTVSLQMNSLKPEDTAVYYCAAVRDYVGMPYYSGSA | |
| | YEYWGQGTQVTVSSGGGGSGLNDIFEAQKIEWHEGGGGSGGGGSL | |
| | PETGG | |
| 134 | MMGVKVLFALICIAVAEASQFRVSPLDRTWNLGETVELKCQVLLS | 851 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGGG | |
| | GSGGGGSQVQLVESGGGLVQAGGSLRLSCAASGLTFSSYGMAWFR | |
| | RALGREREFVGAIRPGTIPYYSESVKGRFTVSKDNAKNTVSLQMNS | |
| | LKPEDTAVYYCAAVRDYVGMPYYSGSAYEYWGQGTQVTVSSGSG | |
| | HHHHHHHHHHRIRRSGSGVKQTLNFDLLKLAGDVESNPGPMMGV | |
| | KVLFALICIAVAEAEVQLVESGGGPVQAGGSLRLSCAASGRTYRGYS | |
| | MAWFRQSPGKEREFVAAIVWSDGNTYYEDFVKGRFTISRDSAKNT | |
| | LYLQMTNLKPEDTALYYCAAKIRPYIFKIAGQYDYWGQGTQVTVSS | |
| | GGGGSGGGGSEVQLVESGGGPVQAGGSLRLSCAASGRTYRGYSMA | |
| | WFRQSPGKEREFVAAIVWSDGNTYYEDFVKGRFTISRDSAKNTLYL | |
| | QMTNLKPEDTALYYCAAKIRPYIFKIAGQYDYWGQGTQVTVSSGGG | |
| | GSAGVTQSPTHLIKTRGQQVTLRCSPKSGHDTVSWYQQALGQGPQ | |
| | FIFQYYEEEERQRGNFPDRFSGHQFPNYSSELNVNALLLGDSALYLC | |
| | ASSLGEGRVDGYTFGSGTRLTVVEDLKNVFPPEVAVFEPSKAEISRT | |
| | QKATLVCLATGFYPPHVELSWWVNGKEVHDGVCTDPQPLKEQPA | |
| | LQDSRYALSSRLRVSATFWQDPRNHFRCQVQFYGLSENDEWTQDR | |
| | AKPVTQIVSAEAWGRADCGGGGSGGGGSQVQLVESGGGLVQAGG | |
| | SLRLSCAASGLTFSSYGMAWFRRALGREREFVGAIRPGTIPYYSESV | |
| | KGRFTVSKDNAKNTVSLQMNSLKPEDTAVYYCAAVRDYVGMPYY | |
| | SGSAYEYWGQGTQVTVSSGGGGSGLNDIFEAQKIEWHEGGGGSGG | |
| | GGSLPETGG | |
| 135 | MMGVKVLFALICIAVAEASQFRVSPLDRTWNLGETVELKCQVLLS | 852 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSSQFRVSPLDRTWNLGETVELKCQVLLSNPTSGCSWL | |
| | FQPRGAAASPTFLLYLSQNKPKAAEGLDTQRFSGKRLGDTFVLTLS | |
| | DFRRENEGCYFCSALSNSIMYFSHFVPVFLPAGGGGSGGGGSGGGG | |
| | SGGGGSLAKTTQPISVDSYEGQEVNITCSHNNIATNDYITWYQQF | |
| | PSQGPRFIIQGYKTKVTNEVASLFIPADRKSSTLSLPRVSLSDTAV | |
| | YYCLVGDMDQAGTALIFGKGTTLSVSSDYIQNPDPAVYQLRDS | |
| | KSSDKFVCLFTDFDSQIQVSQSKDSDVYITDKCVLDMRSMDFKS | |
| | NSAVAWSQKSDFTCANAFQNSIIPEDTFFPSPESSCGGGGSGGGG | |
| | SEVRLQQSGPDLIKPGASVKMSCKASGYTFTGYVMHWVYKQRPG | |
| | QGLEWIGFINPYNDDIQSNERFRGKATLTSDKSSTTAYMELSSLTSE | |
| | DSAVYYCARGAGYNFDGAYRFEDFWGQGTTLTVSSGGGGSGGGG | |
| | SGGGGSDVVMTQSPLSLPYSLGDQASISCRSSQRLVHSNGNTYLHW | |
| | YLQKPGQSPKLLIYRVSNRFPGVPDRFSGSGSGTDFTLKISRVEAED | |
| | LGIYFCSQSTHVPYTFGGGTKLEIKRGSGHHHHHHHHHHRIRRSGS | |
| | GVKQTLNFDLLKLAGDVESNPGPMMGVKVLFALICIAVAEAAGVT | |
| | QSPTHLIKTRGQQVTLRCSPKSGHDTVSWYQQALGQGPQFIFQYYE | |
| | EEERQRGNFPDRFSGHQFPNYSSELNVNALLLGDSALYLCASSLGE | |
| | GRVDGYTFGSGTRLTVVEDLKNVFPPEVAVFEPSKAEISRTQKATL | |
| | VCLATGFYPPHVELSWWVNGKEVHDGVCTDPQPLKEQPALQDSR | |
| | YALSSRLRVSATFWQDPRNHFRCQVQFYGLSENDEWTQDRAKPVT | |
| | QIVSAEAWGRADCGGGGSGGGGSEVQLVESGGGPVQAGGSLRLSC | |
| | AASGRTYRGYSMAWFRQSPGKEREFVAAIVWSDGNTYYEDFVKGR | |
| | FTISRDSAKNTLYLQMTNLKPEDTALYYCAAKIRPYIFKIAGQYDYW | |
| | GQGTQVTVSSGGGGSGLNDIFEAQKIEWHEGGGGSGGGGSLPETG | |
| | G | |
| 136 | MMGVKVLFALICIAVAEASQFRVSPLDRTWNLGETVELKCQVLLS | 853 |
| | NPTSGCSWLFQPRGAAASPTFLLYLSQNKPKAAEGLDTQRFSGKRL | |
| | GDTFVLTLSDFRRENEGCYFCSALSNSIMYFSHFVPVFLPAGGGGSG | |
| | GGGSGGGGSVLQQTPAYIKVQTNKMVMLSCEAKISLSNMRIYWLR | |
| | QRQAPSSDSHHEFLALWDSAKGTIHGEEVEQEKIAVFRDASRFILNL | |
| | TSVKPEDSGIYFCMIVGSPELTFGKGTQLSVVDFLPTTAGGGGSGGG | |
| | GSGGGGSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDYIQNPDPAV | |
| | YQLRDSKSSDKFVCLFTDFDSQIQVSQSKDSDVYITDKCVLDMR | |
| | SMDFKSNSAVAWSQKSDFTCANAFQNSIIPEDTFFPSPESSCGGG | |
| | GSGGGGSEVRLQQSGPDLIKPGASVKMSCKASGYTFTGYVMHWV | |
| | YKQRPGQGLEWIGFINPYNDDIQSNERFRGKATLTSDKSSTTAYME | |
| | LSSLTSEDSAVYYCARGAGYNFDGAYRFEDFWGQGTTLTVSSGGG | |
| | GSGGGGSGGGGSDVVMTQSPLSLPYSLGDQASISCRSSQRLVHSNG | |
| | NTYLHWYLQKPGQSPKLLIYRVSNRFPGVPDRFSGSGSGTDFTLKIS | |
| | RVEAEDLGIYFCSQSTHVPYTFGGGTKLEIKRGSGHHHHHHHHHHR | |
| | IRRSGSGVKQTLNFDLLKLAGDVESNPGPMMGVKVLFALICIAVAE | |
| | AAGVTQSPTHLIKTRGQQVTLRCSPKSGHDTVSWYQQALGQGPQFI | |
| | FQYYEEEERQRGNFPDRFSGHQFPNYSSELNVNALLLGDSALYLCA | |
| | SSLGEGRVDGYTFGSGTRLTVVEDLKNVFPPEVAVFEPSKAEISRTQ | |
| | KATLVCLATGFYPPHVELSWWVNGKEVHDGVCTDPQPLKEQPAL | |
| | QDSRYALSSRLRVSATFWQDPRNHFRCQVQFYGLSENDEWTQDRA | |
| | KPVTQIVSAEAWGRADCGGGGSGGGGSEVQLVESGGGPVQAGGSL | |
| | RLSCAASGRTYRGYSMAWFRQSPGKEREFVAAIVWSDGNTYYEDF | |
| | VKGRFTISRDSAKNTLYLQMTNLKPEDTALYYCAAKIRPYIFKIAGQ | |
| | YDYWGQGTQVTVSSGGGGSGLNDIFEAQKIEWHEGGGGSGGGGSL | |
| | PETGG | |
| 137 | MMGVKVLFALICIAVAEADLAKTTQPISVDSYEGQEVNITCSHNN | 854 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGGGGSGGGGSEVQLVESGGGPVQAGGSLRLSCAASGRTYR | |
| | GYSMAWFRQSPGKEREFVAAIVWSDGNTYYEDFVKGRFTISRDSA | |
| | KNTLYLQMTNLKPEDTALYYCAAKIRPYIFKIAGQYDYWGQGTQVT | |
| | VSSGSGHHHHHHHHHHRIRRSGSGVKQTLNFDLLKLAGDVESNPG | |
| | PMMGVKVLFALICIAVAEAAGVTQSPTHLIKTRGQQVTLRCSPKSG | |
| | HDTVSWYQQALGQGPQFIFQYYEEEERQRGNFPDRFSGHQFPNYSS | |
| | ELNVNALLLGDSALYLCASSLGEGRVDGYTFGSGTRLTVVEDLKN | |
| | VFPPEVAVFEPSKAEISRTQKATLVCLATGFYPPHVELSWWVNGKE | |
| | VHDGVCTDPQPLKEQPALQDSRYALSSRLRVSATFWQDPRNHFRC | |
| | QVQFYGLSENDEWTQDRAKPVTQIVSAEAWGRADCGGGGSGGGG | |
| | SEVQLVESGGGPVQAGGSLRLSCAASGRTYRGYSMAWFRQSPGKE | |
| | REFVAAIVWSDGNTYYEDFVKGRFTISRDSAKNTLYLQMTNLKPED | |
| | TALYYCAAKIRPYIFKIAGQYDYWGQGTQVTVSSGGGGSGLNDIFEA | |
| | QKIEWHEGGGGSGGGGSLPETGG | |
| 138 | MMGVKVLFALICIAVAEADLAKTTQPISVDSYEGQEVNITCSHNN | 855 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDYI | |
| | QNPDPAVYQLRDSKSSDKFVCLFTDFDSQIQVSQSKDSDVYITD | |
| | KCVLDMRSMDFKSNSAVAWSQKSDFTCANAFQNSIIPEDTFFPS | |
| | PESSCGGGGSGGGGSEVQLVESGGGPVQAGGSLRLSCAASGRTYR | |
| | GYSMAWFRQSPGKEREFVAAIVWSDGNTYYEDFVKGRFTISRDSA | |
| | KNTLYLQMTNLKPEDTALYYCAAKIRPYIFKIAGQYDYWGQGTQVT | |
| | VSSGSGHHHHHHHHHHRIRRSGSGVKQTLNFDLLKLAGDVESNPG | |
| | PMMGVKVLFALICIAVAEAAGVTQSPTHLIKTRGQQVTLRCSPKSG | |
| | HDTVSWYQQALGQGPQFIFQYYEEEERQRGNFPDRFSGHQFPNYSS | |
| | ELNVNALLLGDSALYLCASSLGEGRVDGYTFGSGTRLTVVEDLKN | |
| | VFPPEVAVFEPSKAEISRTQKATLVCLATGFYPPHVELSWWVNGKE | |
| | VHDGVCTDPQPLKEQPALQDSRYALSSRLRVSATFWQDPRNHFRC | |
| | QVQFYGLSENDEWTQDRAKPVTQIVSAEAWGRADCGGGGSGGGG | |
| | SEVRLQQSGPDLIKPGASVKMSCKASGYTFTGYVMHWVYKQRPG | |
| | QGLEWIGFINPYNDDIQSNERFRGKATLTSDKSSTTAYMELSSLTSE | |
| | DSAVYYCARGAGYNFDGAYRFEDFWGQGTTLTVSSGGGGSGGGG | |
| | SGGGGSDVVMTQSPLSLPYSLGDQASISCRSSQRLVHSNGNTYLHW | |
| | YLQKPGQSPKLLIYRVSNRFPGVPDRFSGSGSGTDFTLKISRVEAED | |
| | LGIYFCSQSTHVPYTFGGGTKLEIKRGGGGSGLNDIFEAQKIEWHEG | |
| | GGGSGGGGSLPETGG | |
| 139 | MMGVKVLFALICIAVAEADEVQLVESGGGPVQAGGSLRLSCAASG | 856 |
| | RTYRGYSMAWFRQSPGKEREFVAAIVWSDGNTYYEDFVKGRFTISR | |
| | DSAKNTLYLQMTNLKPEDTALYYCAAKIRPYIFKIAGQYDYWGQGT | |
| | QVTVSSGGGGSLAKTTQPISVDSYEGQEVNITCSHINNIATNDYIT | |
| | WYQQFPSQGPRFIIQGYKTKVTNEVASLFIPADRKSSTLSLPRVS | |
| | LSDTAVYYCLVGDMDQAGTALIFGKGTTLSVSSDGGGGSGGGG | |
| | SGGGGSAGVTQSPTHLIKTRGQQVTLRCSPKSGHDTVSWYQQALG | |
| | QGPQFIFQYYEEEERQRGNFPDRFSGHQFPNYSSELNVNALLLGDS | |
| | ALYLCASSLGEGRVDGYTFGSGTRLTVVGGGGSGGGGSEVQLVES | |
| | GGGPVQAGGSLRLSCAASGRTYRGYSMAWFRQSPGKEREFVAAIV | |
| | WSDGNTYYEDFVKGRFTISRDSAKNTLYLQMTNLKPEDTALYYCAA | |
| | KIRPYIFKIAGQYDYWGQGTQVTVSSGGGGSGLNDIFEAQKIEWHE | |
| | GGGGSGGGGSLPETGGHHHHHHHHHH | |
| 140 | MMGVKVLFALICIAVAEADEVRLQQSGPDLIKPGASVKMSCKASG | 857 |
| | YTFTGYVMHWVYKQRPGQGLEWIGFINPYNDDIQSNERFRGKATL | |
| | TSDKSSTTAYMELSSLTSEDSAVYYCARGAGYNFDGAYRFEDFWG | |
| | QGTTLTVSSGGGGSGGGGSGGGGSDVVMTQSPLSLPYSLGDQASIS | |
| | CRSSQRLVHSNGNTYLHWYLQKPGQSPKLLIYRVSNRFPGVPDRFS | |
| | GSGSGTDFTLKISRVEAEDLGIYFCSQSTHVPYTFGGGTKLEIKRGG | |
| | GGSLAKTTQPISVDSYEGQEVNITCSHNNIATNDYITWYQQFPSQ | |
| | GPRFIIQGYKTKVTNEVASLFIPADRKSSTLSLPRVSLSDTAVYY | |
| | CLVGDMDQAGTALIFGKGTTLSVSSDGGGGSGGGGSGGGGSAG | |
| | VTQSPTHLIKTRGQQVTLRCSPKSGHDTVSWYQQALGQGPQFIFQY | |
| | YEEEERQRGNFPDRFSGHQFPNYSSELNVNALLLGDSALYLCASSL | |
| | GEGRVDGYTFGSGTRLTVVGGGGSGLNDIFEAQKIEWHEGGGGSG | |
| | GGGSLPETGGHHHHHHHHHH | |
| 141 | MMGVKVLFALICIAVAEADEVRLQQSGPDLIKPGASVKMSCKASG | 858 |
| | YTFTGYVMHWVYKQRPGQGLEWIGFINPYNDDIQSNERFRGKATL | |
| | TSDKSSTTAYMELSSLTSEDSAVYYCARGAGYNFDGAYRFEDFWG | |
| | QGTTLTVSSGGGGSGGGGSGGGGSDVVMTQSPLSLPYSLGDQASIS | |
| | CRSSQRLVHSNGNTYLHWYLQKPGQSPKLLIYRVSNRFPGVPDRFS | |
| | GSGSGTDFTLKISRVEAEDLGIYFCSQSTHVPYTFGGGTKLEIKRGG | |
| | GGSLAKTTQPISVDSYEGQEVNITCSHNNIATNDYITWYQQFPSQ | |
| | GPRFIIQGYKTKVTNEVASLFIPADRKSSTLSLPRVSLSDTAVYY | |
| | CLVGDMDQAGTALIFGKGTTLSVSSDGGGGSGGGGSGGGGSAG | |
| | VTQSPTHLIKTRGQQVTLRCSPKSGHDTVSWYQQALGQGPQFIFQY | |
| | YEEEERQRGNFPDRFSGHQFPNYSSELNVNALLLGDSALYLCASSL | |
| | GEGRVDGYTFGSGTRLTVVGGGGSEVRLQQSGPDLIKPGASVKMS | |
| | CKASGYTFTGYVMHWVYKQRPGQGLEWIGFINPYNDDIQSNERFR | |
| | GKATLTSDKSSTTAYMELSSLTSEDSAVYYCARGAGYNFDGAYRF | |
| | EDFWGQGTTLTVSSGGGGSGGGGSGGGGSDVVMTQSPLSLPYSLG | |
| | DQASISCRSSQRLVHSNGNTYLHWYLQKPGQSPKLLIYRVSNRFPG | |
| | VPDRFSGSGSGTDFTLKISRVEAEDLGIYFCSQSTHVPYTFGGGTKL | |
| | EIKRGGGGSGLNDIFEAQKIEWHEGGGGSGGGGSLPETGGHHHHH | |
| | HHHHH | |
| 142 | MMGVKVLFALICIAVAEADEVRLQQSGPDLIKPGASVKMSCKASG | 859 |
| | YTFTGYVMHWVYKQRPGQGLEWIGFINPYNDDIQSNERFRGKATL | |
| | TSDKSSTTAYMELSSLTSEDSAVYYCARGAGYNFDGAYRFEDFWG | |
| | QGTTLTVSSGGGGSGGGGSGGGGSDVVMTQSPLSLPYSLGDQASIS | |
| | CRSSQRLVHSNGNTYLHWYLQKPGQSPKLLIYRVSNRFPGVPDRFS | |
| | GSGSGTDFTLKISRVEAEDLGIYFCSQSTHVPYTFGGGTKLEIKRGG | |
| | GGSLAKTTQPISVDSYEGQEVNITCSHNNIATNDYITWYQQFPSQ | |
| | GPRFIIQGYKTKVTNEVASLFIPADRKSSTLSLPRVSLSDTAVYY | |
| | CLVGDMDQAGTALIFGKGTTLSVSSDGGGGSGGGGSGGGGSAG | |
| | VTQSPTHLIKTRGQQVTLRCSPKSGHDTVSWYQQALGQGPQFIFQY | |
| | YEEEERQRGNFPDRFSGHQFPNYSSELNVNALLLGDSALYLCASSL | |
| | GEGRVDGYTFGSGTRLTVVGGGGSGGGGSEVQLVESGGGPVQAG | |
| | GSLRLSCAASGRTYRGYSMAWFRQSPGKEREFVAAIVWSDGNTYYE | |
| | DFVKGRFTISRDSAKNTLYLQMTNLKPEDTALYYCAAKIRPYIFKIA | |
| | GQYDYWGQGTQVTVSSGGGGSGLNDIFEAQKIEWHEGGGGSGGGG | |
| | SLPETGGHHHHHHHHHH | |
| 143 | MMGVKVLFALICIAVAEAEVRLQQSGPDLIKPGASVKMSCKASGY | 860 |
| | TFTGYVMHWVYKQRPGQGLEWIGFINPYNDDIQSNERFRGKATLT | |
| | SDKSSTTAYMELSSLTSEDSAVYYCARGAGYNFDGAYRFEDFWGQ | |
| | GTTLTVSSGGGGSLAKTTQPISVDSYEGQEVNITCSHNNIATNDYI | |
| | TWYQQFPSQGPRFIIQGYKTKVTNEVASLFIPADRKSSTLSLPRV | |
| | SLSDTAVYYCLVGDMDQAGTALIFGKGTTLSVSSDGGGGSGGG | |
| | GSGGGGSAGVTQSPTHLIKTRGQQVTLRCSPKSGHDTVSWYQQAL | |
| | GQGPQFIFQYYEEEERQRGNFPDRFSGHQFPNYSSELNVNALLLGD | |
| | SALYLCASSLGEGRVDGYTFGSGTRLTVVGGGGSDVVMTQSPLSLP | |
| | YSLGDQASISCRSSQRLVHSNGNTYLHWYLQKPGQSPKLLIYRVSN | |
| | RFPGVPDRFSGSGSGTDFTLKISRVEAEDLGIYFCSQSTHVPYTFGG | |
| | GTKLEIKRGSGGLNDIFEAQKIEWHEGSGHHHHHHHHHH | |
| 144 | MMGVKVLFALICIAVAEADLAKTTQPISVDSYEGQEVNITCSHNN | 861 |
| | IATNDYITWYQQFPSQGPRFIIQGYKTKVTNEVASLFIPADRKSS | |
| | TLSLPRVSLSDTAVYYCLVGDMDQAGTALIFGKGTTLSVSSDGG | |
| | GGSEVRLQQSGPDLIKPGASVKMSCKASGYTFTGYVMHWVYKQR | |
| | PGQGLEWIGFINPYNDDIQSNERFRGKATLTSDKSSTTAYMELSSLT | |
| | SEDSAVYYCARGAGYNFDGAYRFEDFWGQGTTLTVSSGGGGSGG | |
| | GGSGGGGSDVVMTQSPLSLPYSLGDQASISCRSSQRLVHSNGNTYL | |
| | HWYLQKPGQSPKLLIYRVSNRFPGVPDRFSGSGSGTDFTLKISRVEA | |
| | EDLGIYFCSQSTHVPYTFGGGTKLEIKRGGGGSAGVTQSPTHLIKTR | |
| | GQQVTLRCSPKSGHDTVSWYQQALGQGPQFIFQYYEEEERQRGNF | |
| | PDRFSGHQFPNYSSELNVNALLLGDSALYLCASSLGEGRVDGYTFG | |
| | SGTRLTVVGSGGLNDIFEAQKIEWHEGSGHHHHHHHHHH | |

### Example 8. Single Chain Bispecific sTCR Construct Design and Testing

For therapeutic applications of the multispecific multimeric soluble TCRs, it was necessary to design the multispecific molecule such that it can be delivered in vivo effectively with controlled and predictable outcome. One consideration was to be able to deliver the composition as recombinant nucleic acid, e.g., DNA or RNA. Plasmids and vectors for DNA delivery are well known and can be suitably used. RNA can be efficiently manufactured and predictably dosed and delivered. Additionally, in order to avoid the unpredictability of two TCR chains being available in the suitable concentration for pairing effectively in vivo, a single mRNA needs to be designed to encode the entire polypeptide, where the TCR chains are translated simultaneously in vivo, along with a second engager, e.g. a T cell engager as variously exemplified herein. Yet another consideration was weighed in, wherein the designs for the multimeric compositions to be encoded comprised a single mRNA, but additionally incorporated one or more cleavable sequence elements and linkers, where the long polypeptide may be cleaved upon translation to effectively engage without experiencing steric hindrance. Various design modifications were explored and tested as detailed herein.

In this Example, protein structure guided soluble TCR constructs were designed to incorporate a linker between anti-CD3 binder and TRBV to orient the anti-CD3 scFv. In one exemplary design, suitable constructs were designed using scaffolds based on platform scaffolds 74, 107, 109, 111 or 113 described earlier. Exemplary soluble TCR alpha-beta chains were designed, having an N terminal to C-terminal orientation where beta chains appear N-terminal to an alpha chain. Conversely, exemplary soluble TCR alpha beta chains were designed where the alpha chain appears N terminal to the beta chain. (Table 12). Additionally, BiTEs were designed with these variations, including a variation where T cell engagers (e.g., anti CD3 engagers, e.g., an anti-CD3 scFV as described above) were designed as an N-terminal subunit connected to the TCR chains with linkers, or as an internal moiety connected to individual TCR chains, whereas spatially occupying the same three-dimensional structure with respect to TCR chains, where it is connected to one or the other chain vial linkers, as well as whether it is terminally placed on the polypeptide or internally placed.

Use of protein structure-guided design to engineer flexible glycine-serine linkers to physically link the extracellular domains of TCRα and TCRβ with an anti-CD3 single-chain variable fragment (scFv) to generate soluble TCR (sTCR) BiTEs encoded by a single mRNA or DNA construct. The TCRα and TCRβ extracellular domains (including the variable and constant domains) are liberated from the cell membrane via truncation precisely 5′ of the conserved cysteine residues at the end of the TCRα and TCRβ constant domains. The TCRα, TCRβ, and anti-CD3 scFv components were linked in two different orientations as explained earlier—TCRαβ or TCRβα. To determine approximate, compatible glycine-serine linker lengths, experimental TCR crystal structures solved in-house were used to visualize and measure distances in angstroms between the N- and C-termini of the extracellular portions of the TCRα and TCRβ chains. Glycine-serine linkers in increasing increments of (GGGGS)n were built between the chains and the resulting fusion proteins with anti-CD3 scFvs were modeled via AlphaFold2 to determine the linker lengths that minimally bridged the distances between desired termini without disrupting the three-dimensional conformation of the TCRαβ heterodimer. For the TCRαβ orientation, a minimal linker of 20 amino acids—(GGGGS)4—was constructed to join the C-terminus of the TCRα chain to the N-terminus of the anti-CD3 scFv, which was in turn joined via a 5 amino acid linker—(GGGGS)— from the C-terminus of the scFv to the N-terminus of the TCRβ chain. For the TCRβα orientation, the anti-CD3 scFv is at the N-terminus of the construct and is joined at the C-terminus via a 5 amino acid linker—(GGGGS)—to the N-terminus of the TCRβ chain followed by a minimal linker of 35 amino acids—(GGGGS)7—to join the C-terminus of the TCRβ chain to the N-terminus of the TCRα chain. The sequences below also contain secretion signal sequences, Ni-NTA histidine affinity tags and site-specific sortase conjugation tags for secretion of the expressed protein out of the cell, purification and conjugation, respectively.

Exemplary designs are shown in FIGS. 35A and 35B. In one design, shown in FIG. 35A, the TCR and linker orientation leaves a free N terminal anti-CD3 scFv. The TCRs are oriented in beta chain-alpha chain manner from N- to C-termini. Shown in FIG. 35B is another design where the anti-CD3 scFv can access the same position as in the design shown in FIG. 35A, but with a shorter linker.

Various linkers were used to evaluate their roles in the effectiveness of the sTCR construct functions. For example, GS linkers (comprising Glycine and Serine residues, 35 residues long) were used in making and testing Ros9a_βα_GS constructs, where Ros9a scaffold was used as a backbone, the TCR was oriented from N terminal to C-terminal:beta chain-alpha chain. GS linkers were also used in making and testing Ros9a_αβ_GS constructs, where Ros9a scaffold was used as a backbone, the TCR was oriented from N terminal to C-terminal:alpha chain-beta chain. Exemplary structures are shown graphically in FIGS. 23 and 24 respectively. An exemplary sTCR construct of the Ros9a_βα_GS type is Ros9a_SC107wβα_antiCD3 (SEQ ID NO: 862). An exemplary sTCR construct of the Ros9a_αβ_GS construct type is Ros9aSC111wβα_antiCD3 (SEQ ID NO: 867). (Table 12).

In a parallel study, a 37 residue linker (Linker 1) was used in making and testing Ros9a_βα_Linker 1 construct, where Ros9a scaffold was used as a backbone, the TCR was oriented from N terminal to C-terminal:beta chain, followed by alpha chain. The linkers were also used in the making and testing Ros9a_αβ_Linker 1 construct, where Ros9a scaffold was used as a backbone, the TCR was oriented from N terminal to C-terminal:alpha chain-beta chain, following similar designs as shown for the GS linkers in FIGS. 23 and 24 respectively.

In another study, a 36 residue linker (Linker 2) from phage pIII protein was used in making and testing Ros9a_βα_Linker 2 construct, where Ros9a scaffold was used as a backbone, the TCR was oriented from N terminal to C-terminal:beta chain-alpha chain; or Ros9a_αβ_Linker 2 construct, where alpha and beta the sTCR alpha and beta chains were oriented from N terminal to C-terminal: alpha chain-beta chain. Table 11 summarizes the constructs designed.

TABLE 11
BiTE Constructs and Domains

| | TCR | | |
|---|---|---|---|
| Construct | orientation | Linker | Exemplary Linker Sequences |
| Ros9aSCβα_ | βα | 35 residues | GGGGSGGGGSGGGGSGGGGSGGG |
| 35GS Linker | | | GSGGGGSGGGGS (G₄S)₇ (SEQ ID |
| | | | NO: 864) |
| Ros9a_αβ_ | αβ | 35 residues | GGGGSGGGGSGGGGSGGGGSGGG |
| 35GS Linker | | | GSGGGGSGGGGS (G₄S)₇ (SEQ ID |
| | | | NO: 864) |
| Ros9a_βα_ | βα | 37 residues | GGSSGSG-X₂₅-GSGSG, where X is an |
| Linker 1 | | | amino acid selected from A, G, S and T. |
| | | | (SEQ ID NO: 872) |
| Ros9a_αβ_ | αβ | 37 residues | GGSSGSG-X₂₅-GSGSG, where X is |
| Linker 1 | | | an amino acid selected from A, G, S |
| | | | and T (SEQ ID NO: 872) |
| Ros9a_βα_ | βα | 36 residues | SGGGSGGGSEGGGSEGGGSEGGGS |
| Linker 2 | | | EGGGSGGGSG (SEQ ID NO: 873) |
| Ros9a_αβ_ | αβ | 36 residues | SGGGSGGGSEGGGSEGGGSEGGGS |
| Linker 2 | | | EGGGSGGGSG(SEQ ID NO: 873) |

The respective sequences and individual domains of the single chain sTCR constructs are detailed in Table 12. In some constructs certain amino acid positions are mutated for increasing stability of the soluble TCR polypeptide. For example, S139F, T150I and A190T mutations can be incorporated in TRAC fragment in various constructs, the positions are indicated by *sign right after the amino acid residue that can be mutated and shown in its non-mutated versions in the Table 12 below. Similarly for TRBC, several mutations have been reported to enhance stability, which were prepared for testing, including E134K, H139R, D155P, or S1701D. Additionally, one or more N-glycosylation sites may be mutated from TRAC/TRBC (e.g., the Asparagine residue underlined and bolded in some sequences shown in Table 12). However, single chain soluble TCR structures may lack several TRAC modifications, for example S139F, T150I and A190T mutations; and are still found to be stable and efficient in activity at least as compared to the corresponding recombinant TCRs with the stability enhancing modifications described above. Similarly, single chain soluble TCR structures may lack TRBC modifications, for example E134K, H139R, D155P, or S1701D. (See, for example FIG. 40). AlphaFold 2 modeling informs linker length decisions. In some embodiments, the multispecific molecule comprises one or more sequences of SEQ ID NO: 604, SEQ ID NO: 682, SEQ ID NO: 603, (SEQ ID NO: 683) (GGGGS), SEQ ID NO: 620, SEQ ID NO: 863, SEQ ID NO: 864, SEQ ID NO: 619, SEQ ID NO: 865, and SEQ ID NO: 866. In some embodiments, the multispecific molecule comprises a sequence of SEQ ID NO: 862. In some embodiments, the multispecific molecule comprises a sequence of SEQ ID NO: 862 without a signal peptide of SEQ ID NO: 668. In some embodiments, wherein the multispecific molecule comprises one or more sequences selected from the group consisting of SEQ ID NO: 619, SEQ ID NO: 868, SEQ ID NO: 869, SEQ ID NO: 604, SEQ ID NO: 870, SEQ ID NO: 603, SEQ ID NO:620, SEQ ID NO: 871, and SEQ ID NO: 866. In some embodiments, the multispecific molecule comprises a sequence of SEQ ID NO: 867. In some embodiments, the multispecific molecule comprises a sequence of SEQ ID NO: 867, but without the signal peptide sequence of SEQ ID NO: 667.

TABLE 12
Single Chain Bispecific Structure Sequences and Domains

| | Amino Acid Sequence | |
|---|---|---|
| TCRβα | MMGVKVLFALICIAVAEAAIQMTQSPSSLSASVGDRVTITCRASQDIRN | |
| single- | YLNWYQQKPGKAPKLLIYYTSRLESGVPSRFSGSGSGTDYTLTISSLQP | |
| chain | EDFATYYCQQGNTLPWTFGQGTKVEIKGGGGSGGGGSGGGGSGGGGS | |
| sTCR | GGGSEVQLVESGGGLVQPGGSLRLSCAASGYSFTGYTMNWVRQAPGK | |
| BiTE | GLEWVALINPYKGVSTYNQKFKDRFTISVDKSKNTAYLQMNSLRAED | |
| Full | TAVYYCARSGYYGDSDWYFDVWGQGTLVTVSSGGGGSAGVTQSPTH | |
| Sequence | LIKTRGQQVTLRCSPKSGHDTVSWYQQALGQGPQFIFQYYEEEERQRG | |
| of sTCR | NFPDRFSGHQFPNYSSELNVNALLLGDSALYLCASSLGEGRVDGYTFG | |
| Scaffold | SGTRLTVVEDLKNVFPPEVAVFEPSE*AEISH*TQKATLVCLATGFYPD* | |
| name: | HVELSWWVNGKEVHS*GVCTDPQPLKEQPALQDSRYALSSRLRVSAT | |
| Ros9a_ | FWQNPRNHFRCQVQFYGLSENDEWTQDRAKPVTQIVSAEAWGRADG | |
| SC107wβα_ | GGGSGGGGSGGGGSGGGGSGGGGSGGGGSGGGGSLAKTTQPISVDSY | |
| antiCD3 | EGQEVNITCSHNNIATNDYITWYQQFPSQGPRFIIQGYKTKVTNEVASL | |
| | FIPADRKSSTLSLPRVSLSDTAVYYCLVGDMDQAGTALIFGKGTTLSVS | |
| | SDYIQNPDPAVYQLRDSKSSDKS*VCLFTDFDSQT*QVSQSKDSDVYIT | |
| | DKCVLDMRSMDFKSNSAVAWSQKSDFA*CANAFQNSIIPEDTFFPSPES | |
| | SGSGLPETGGGSGHHHHHHHHHH (SEQ ID NO: 862) | |
| | Signal | MMGVKVLFALICIAVAEA (SEQ ID NO: 668) |
| | Peptide | |
| | Anti- | AIQMTQSPSSLSASVGDRVTITCRASQDIRNYLNWYQQKPGK |
| | CD3- | APKLLIYYTSRLESGVPSRFSGSGSGTDYTLTISSLQPEDFATY |
| | (scFv) | YCQQGNTLPWTFGQGTKVEIK (SEQ ID NO: 604) |
| | VL | LC CDR1 QDIRNY (SEQ ID NO: 732), |
| | | LC CDR2 YTS (SEQ ID NO: 733), |
| | | LC CDR3 QQGNTLPWT (SEQ ID NO: 610). (IMGT numbering |
| | | scheme) |
| | linker | GGGGSGGGGSGGGGSGGGGSGGGS (SEQ ID NO: 682) |
| | between | |
| | anti-CD3 | |
| | VL and | |
| | VH | |
| | Anti- | EVQLVESGGGLVQPGGSLRLSCAASGYSFTGYTMNWVRQA |
| | CD3- | PGKGLEWVALINPYKGVSTYNQKFKDRFTISVDKSKNTAYL |
| | (ScFv) | QMNSLRAEDTAVYYCARSGYYGDSDWYFDVWGQGTLVTV |
| | VH | SS (SEQ ID NO: 603) |
| | | HC CDR1: GYSFTGYT (SEQ ID NO: 729), |
| | | HC CDR2 INPYKGVS (SEQ ID NO: 730), |
| | | HC CDR3 ARSGYYGDSDWYFDV (SEQ ID NO: 731); (IMGT |
| | | numbering scheme) |
| | Linker | GGGGS (SEQ ID NO: 683) |
| | TRBV | AGVTQSPTHLIKTRGQQVTLRCSPKSGHDTVSWYQQALGQG |
| | | PQFIFQYYEEEERQRGNFPDRFSGHQFPNYSSELNVNALLLG |
| | | DSALYLCASSLGEGRVDGYTFGSGTRLTVV (SEQ ID NO: |
| | | 620). |
| | | CDR1: SGHDT (SEQ ID NO: 877) |
| | | CDR2: YYEEEE (SEQ ID NO: 878) |
| | | CDR3: ASSLGEGRVDGYT (SEQ ID NO: 879) |
| | | (Underlined, CDR sequences according to IMGT) |
| | TRBC | EDLKNVFPPEVAVFEPSEAEISHTQKATLVCLATGFYPDHVE |
| | | LSWWVNGKEVHSGVCTDPQPLKEQPALQDSRYALSSRLRVS |
| | | ATFWQNPRNHFRCQVQFYGLSENDEWTQDRAKPVTQIVSA |
| | | EAWGRAD (SEQ ID NO: 863) |
| | (G₄S)₇ | GGGGSGGGGSGGGGSGGGGSGGGGSGGGGSGGGGS (SEQ |
| | | ID NO: 864) |
| | TRAV | LAKTTQPISVDSYEGQEVNITCSHNNIATNDYITWYQQFPSQ |
| | | GPRFIIQGYKTKVTNEVASLFIPADRKSSTLSLPRVSLSDTAV |
| | | YYCLVGDMDQAGTALIFGKGTTLSVSSD (SEQ ID NO: 619). |
| | | CDR1: NIATNDY (SEQ ID NO: 874) |
| | | CDR2: GYKTK (SEQ ID NO: 875) |
| | | CDR3: LVGDMDQAGTALI (SEQ ID NO: 876) |
| | | (Underlined, CDR sequences according to IMGT) |
| | TRAC | YIQNPDPAVYQLRDSKSSDKSVCLFTDFDSQTQVSQSKDSDV |
| | | YITDKCVLDMRSMDFKSNSAVAWSQKSDFACANAFQNSIIP |
| | | EDTFFPSPESS (SEQ ID NO: 865) |
| | HIS-tag | HHHHHHHHHH (SEQ ID NO: 866) |
| TCRαβ | MMGVKVLFALICIAVAEADLAKTTQPISVDSYEGQEVNITCSHNNIATN | |
| single- | DYITWYQQFPSQGPRFIIQGYKTKVTNEVASLFIPADRKSSTLSLPRVSLS | |
| chain | DTAVYYCLVGDMDQAGTALIFGKGTTLSVSSDYIQNPDPAVYQLRDSK | |
| sTCR | SSDKS*VCLFTDFDSQT*QVSQSKDSDVYITDKCVLDMRSMDFKSNSAV | |
| BiTE- | AWSQKSDFA*CANAFQNSIIPEDTFFPSPESSGGGGSGGGGSGGGGSGG | |
| Full | GGSAIQMTQSPSSLSASVGDRVTITCRASQDIRNYLNWYQQKPGKAPKL | |
| sequence | LIYYTSRLESGVPSRFSGSGSGTDYTLTISSLQPEDFATYYCQQGNTLPW | |
| of sTCR | TFGQGTKVEIKGGGGSGGGGSGGGGSGGGGSGGGSEVQLVESGGGLV | |
| Scaffold | QPGGSLRLSCAASGYSFTGYTMNWVRQAPGKGLEWVALINPYKGVST | |
| name: | YNQKFKDRFTISVDKSKNTAYLQMNSLRAEDTAVYYCARSGYYGDSD | |
| Ros9aSC1 | WYFDVWGQGTLVTVSSGGGGSAGVTQSPTHLIKTRGQQVTLRCSPKSG | |
| 11waβ_ | HDTVSWYQQALGQGPQFIFQYYEEEERQRGNFPDRFSGHQFPNYSSELN | |
| antiCD3 | VNALLLGDSALYLCASSLGEGRVDGYTFGSGTRLTVVEDLKNVFPPEV | |
| | AVFEPSE*AEISH*TQKATLVCLATGFYPD*HVELSWWVNGKEVHS*GV | |
| | CTDPQPLKEQPALQDSRYALSSRLRVSATFWQNPRNHFRCQVQFYGLS | |
| | ENDEWTQDRAKPVTQIVSAEAWGRADGSGLPETGGGSGHHHHHHHHH | |
| | H (SEQ ID NO: 867) | |
| | Signal | MMGVKVLFALICIAVAEAD (SEQ ID NO: 667) |
| | Peptide | |
| | TRAV | LAKTTQPISVDSYEGQEVNITCSHNNIATNDYITWYQQFPSQ |
| | | GPRFIIQGYKTKVTNEVASLFIPADRKSSTLSLPRVSLSDTAV |
| | | YYCLVGDMDQAGTALIFGKGTTLSVSSD (SEQ ID NO: 619) |
| | | CDR1: NIATNDY (SEQ ID NO: 874) |
| | | CDR2: GYKTK (SEQ ID NO: 875) |
| | | CDR3: LVGDMDQAGTALI (SEQ ID NO: 876) |
| | | (CDR sequences according to IMGT) |
| | TRAC | YIQNPDPAVYQLRDSKSSDKSVCLFTDFDSQTQVSQSKDSDV |
| | | YITDKCVLDMRSMDFKSNSAVAWSQKSDFACANAFQNSIIP |
| | | EDTFFPSPESS (SEQ ID NO: 868) |
| | (G₄S)₄ | GGGGSGGGGSGGGGSGGGGS (SEQ ID NO: 869) |
| | linker | |
| | Anti- | AIQMTQSPSSLSASVGDRVTITCRASQDIRNYLNWYQQKPGK |
| | CD3 | APKLLIYYTSRLESGVPSRFSGSGSGTDYTLTISSLQPEDFATY |
| | scFv, VL | YCQQGNTLPWTFGQGTKVEIK (SEQ ID NO: 604) |
| | | LC CDR1 QDIRNY (SEQ ID NO: 732), |
| | | LC CDR2 YTS (SEQ ID NO: 733), |
| | | LC CDR3 QQGNTLPWT (SEQ ID NO: 610). (IMGT numbering |
| | | scheme) |
| | GS | GGGGSGGGGSGGGGSGGGGSGGGS (SEQ ID NO: 870) |
| | linker | |
| | VH | EVQLVESGGGLVQPGGSLRLSCAASGYSFTGYTMNWVRQA |
| | | PGKGLEWVALINPYKGVSTYNQKFKDRFTISVDKSKNTAYL |
| | | QMNSLRAEDTAVYYCARSGYYGDSDWYFDVWGQGTLVTV |
| | | SS (SEQ ID NO: 603) |
| | | HC CDR1: GYSFTGYT (SEQ ID NO: 729), |
| | | HC CDR2 INPYKGVS (SEQ ID NO: 730), |
| | | HC CDR3 ARSGYYGDSDWYFDV (SEQ ID NO: 731); (IMGT |
| | | numbering scheme) |
| | TRBV | AGVTQSPTHLIKTRGQQVTLRCSPKSGHDTVSWYQQALGQG |
| | | PQFIFQYYEEEERQRGNFPDRFSGHQFPNYSSELNVNALLLG |
| | | DSALYLCASSLGEGRVDGYTFGSGTRLTVV (SEQ ID NO: 620) |
| | | (Underlined, CDR sequences according to IMGT) |
| | TRBC | EDLKNVFPPEVAVFEPSEAEISHTQKATLVCLATGFYPDHVE |
| | | LSWWVNGKEVHSGVCTDPQPLKEQPALQDSRYALSSRLRVS |
| | | ATFWQNPRNHFRCQVQFYGLSENDEWTQDRAKPVTQIVSA |
| | | EAWGRAD (SEQ ID NO: 871) |
| | HIS-Tag | HHHHHHHHHH (SEQ ID NO: 866) |

Single Chain sTCR Scaffold Design and Cell Surface Expression Test Via pMHC-Multimer Binding Flow Cytometry Assay

The single chain scaffolds generated as shown above were tested for cell surface expression. Results are depicted in the graph in FIG. 38. The construct designs include sTCRs having a peptide linker connecting the TCR alpha and beta chains. GS linker were tested. In parallel, constructs with alpha and beta chain having an alpha-beta as well as in a beta-alpha orientation from N terminus to C terminus were tested. Cell surface expression and peptide binding assays for each construct are shown in the graph at the right side. For expression analysis, 00 of cells in a cell culture transduced with the TCR construct that exhibit TCR expression are shown as % frequency in the graph in FIG. 38. Of note a high percent of cells express single chain sTCR constructs. Next, of the cells that express the sTCRs (taken as 1000%) those that exhibit successful binding of peptide epitope (thereby indicating functional sTCRs) are shown as 00 frequency of TCR expressing cells. The data indicate that nearly 1000% of cells that express the TCR have functional TCRs.

Expression of each construct was verified. Additionally, each of the constructs were verified to be able to bind peptide MHC (pMHC) multimer complexes in vitro. Tumor cell cytotoxicity is assayed as shown in the schematic in FIG. 39A. Appropriate target cells (e.g., cancer cells, or cancel cell lines, in this example A375 cells) were either transduced with HLA or they endogenously express an HLA allele and present the peptide for recognition by sTCR, and cultured in 2D or 3D (spheroid cultures). The cell cultures were them supplemented with soluble TCRs at a concentration between 0.05-100 nM, and T cells (effector) were added. The effector cell to target cell ratio was kept between 0.04:1 to 10:1. In a high throughput assay for sTCR assessment, target cells were transfected to express a fluorescent protein such as GFP or stained with a dye. Rate of killing of target cells presenting the peptide-MHC are assessed by loss of the GFP cells over time by flow cytometry. In one assay using the above method, a comparison of cytotoxicity potential of two constructs were compared (FIG. 39B). One TCR comprised Fc domains tagged to the alpha and beta chains as described previous, along with the anti-CD3 scFv. The other construct in the same Ros9a framework did not include the Fc domains. Data indicated that presence of the Fc domain reduced cytotoxicity potential by about 20-fold. FIGS. 40 and 41 show data of cytotoxicity potential of the constructs based generally on the backbones of scaffolds 74, 107, 111, and 113 (graph) and imaging of GFP cells respectively FIG. 40 shows data of cytotoxicity potential of the constructs Ros9a S74_antiCD3 (top left); Ros9a_SC107wβα_antiCD3 (top right); Ros9aSC111αβ_antiCD3 (bottom left); and Ros9a SC113_antiCD3 (bottom right respectively). Ros9a S74_antiCD3 is a two stranded sTCR structure (alpha and beta chains separate polypeptides), and is used as control to compare the functions of the single chain sTCR constructs Ros9a_SC107wβα_antiCD3; Ros9aSC111αβ_antiCD3; and Ros9a SC113_antiCD3 in each of which the alpha and beta chains are linked by linkers. Ros9a_SC107wβα_antiCD3 has the sequence of SEQ ID NO: 862. Ros9aSC111αβ_antiCD3 and Ros9a SC113_antiCD3 each has about 99% to Ros9aSC111wαβ_antiCD3, (SEQ ID NO: 867). Ros9a SC113_antiCD3 may contain an additional beta chain mutation at position 191, Cys>Ala; compared to Ros9aSC111wαβ_antiCD3. FIG. 41 shows imaging studies of GFP cells in the experiment shown in FIG. 40.

These results show high functional capability of the different soluble TCRs with additional anti-CD3 scFv domains that potentiate T cell mediated killing of tumor cells. The results have demonstrated that TCRs formatted in both the TCRαβ and TCRβα single-chain sTCR aCD3 BiTE format are stably-expressed in a human cell culture protein production system (Expi293) and they are functional in bridging T cells to neoantigen-presenting tumor cells in an in vitro T cell-mediated cytotoxicity assay. In addition, the single-chain sTCR anti-CD3 BiTEs have at least comparable potency relative to multi-chain, unlinked formats using identical TCR sequences.

1. A composition comprising a recombinant nucleic acid for expression in a mammalian cell, the recombinant nucleic acid comprising a sequence encoding a multispecific molecule, wherein the multispecific molecule is an engineered T cell receptor (TCR) construct, comprising:
(i) an MHC-peptide complex engager comprising:
(a) a T cell receptor (TCR) alpha variable (TRAV) domain, and
(b) a TCR beta variable (TRBV) domain; and
(ii) a T cell engager comprising one or more binding domains that bind to an extracellular domain of a receptor expressed by a T cell, wherein the T cell engager comprises an anti-CD3 binding domain comprising a heavy chain variable region (VH) that comprises a heavy chain complementarity determining region 3 (HC CDR3) sequence of ARGIYSDSSDFIGNL (SEQ ID NO: 713) or GIYSDSSDFIGNL (SEQ ID NO: 707).

2. A composition comprising a recombinant nucleic acid comprising a sequence encoding a multispecific molecule, wherein the multispecific molecule comprises:
(i) an MHC-peptide complex engager comprising:
(a) a TCR alpha variable (TRAV) domain, and
(b) a TCR beta variable (TRBV) domain;
wherein the peptide in the MHC-peptide complex comprises a RAS peptide sequence; and
(ii) a T cell engager comprising one or more binding domains that bind to an extracellular domain of a receptor expressed by a T cell, wherein the T cell engager comprises an anti-CD3 binding domain comprising a heavy chain variable region (VH) that comprises a heavy chain complementarity determining region 3 (HC CDR3) sequence of ARGIYSDSSDFIGNL (SEQ ID NO: 713) or GIYSDSSDFIGNL (SEQ ID NO: 707).

3. The composition of claim 2, wherein the multispecific molecule comprises a post-translational modification.

4. The composition of claim 1, wherein the multispecific molecule comprises a mammalian glycosylation signature.

5. The composition of claim 1, wherein the multispecific molecule is soluble.

6. The composition of claim 1, wherein the multispecific molecule lacks a trans-membrane domain.

7. The composition of claim 1, wherein the multispecific molecule consists of two polypeptides, a first polypeptide and a second polypeptide.

8. The composition of claim 7, wherein the first polypeptide and the second polypeptide comprise one or more disulfide bonds between the two polypeptides.

9. The composition of claim 1, wherein the multispecific molecule comprises a first polypeptide and a second polypeptide, wherein the first polypeptide comprises the TRAV domain and the second polypeptide comprises the TRBV domain.

10. The composition of claim 1, wherein the peptide of the MHC-peptide complex is a peptide from a cancer antigen.

11. The composition of claim 2, wherein the peptide of the MHC-peptide complex is a mutant peptide, and wherein the MHC of the MHC-peptide complex binds the mutant peptide with a higher affinity compared to a corresponding wild-type peptide.

12. The composition of claim 1, wherein the peptide of the MHC-peptide complex does not comprise an amino acid sequence YLEPGPVTA.

13. The composition of claim 1, wherein the MHC of the MHC-peptide complex comprises a class I MHC.

14. The composition of claim 1, wherein the MHC of the MHC-peptide complex is a class I MHC polypeptide.

15. The composition of claim 1, wherein the MHC of the MHC-peptide complex is encoded by an HLA that is represented in less than 1% of a human population.

16. The composition of claim 1, wherein the MHC of the MHC-peptide complex has a peptide binding affinity greater than at least 50 nM.

17. The composition of claim 1, wherein the one or more binding domains of the T cell engager bind to an extracellular domain of an endogenous receptor expressed by a T cell.

18. The composition of claim 1, wherein the one or more binding domains of the T cell engager bind to an extracellular domain of a receptor selected from a group consisting of CD3, CD2, CD7, CD5, CD4, CD28, ICAM-1 and CD8.

19. The composition of claim 1, wherein the one or more binding domains of the T cell engager binds to CD3 delta, CD3 gamma or CD3 epsilon.

20. The composition of claim 1, wherein the one or more binding domains of the T cell engager comprises an antibody domain or antigen binding fragment thereof.

21-195. (canceled)
